(19) United States
(12) Patent Application Publication
(10) Pub. No.: US 2024/0182481 A1
(43) Pub. Date: Jun. 6, 2024

(54) PYRROLO[3,2-D]PYRIMIDINE COMPOUNDS AND METHODS OF USE IN THE TREATMENT OF CANCER

(71) Applicant: Tango Therapeutics, Inc., Boston, MA (US)

(72) Inventor: Scott THRONER, Boston, MA (US)

(21) Appl. No.: 18/546,312

(22) PCT Filed: Feb. 15, 2022
(86) PCT No.: PCT/US2022/016441

§ 371 Date: Aug. 14, 2023

Related U.S. Application Data
(60) Provisional application No. 63/149,635, filed on Feb. 15, 2021.

(51) Int. Cl. C07D 487/04; C07D 471/04; C07D 519/00

(52) U.S. Cl. CPC C07D 487/04; C07D 471/04; C07D 519/00

(57) ABSTRACT

Compounds are provided according to Formula (I), and pharmaceutically acceptable salts, hydrates, solvates, prodrugs, tautomers and stereoisomers, as well as pharmaceutical compositions, wherein Ring B, Ring A, R¹, R², R⁶, L, X¹ and X² are as defined herein. The compounds described herein are contemplated to be useful for the prevention and treatment of a variety of conditions.

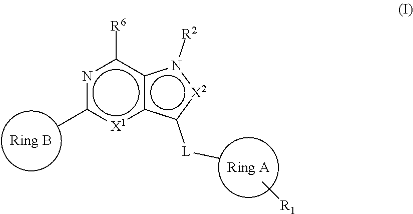

### CROSS-REFERENCE TO RELATED APPLICATION

This application claims the benefit of and priority to U.S. provisional patent application No. 63/149,635, filed Feb. 15, 2021, the contents of which are incorporated herein by reference in its entirety.

### BACKGROUND

Ubiquitin is a small, highly conserved protein composed of 76 amino acids that is post-transcriptionally attached to target proteins, including itself, via a concerted three-step enzymatic reaction. This covalent linkage or isopeptide bond primarily occurs between the C-terminal glycine of ubiquitin and the ε-amino group of lysine residue(s) on the target protein (Pickart, C. M., Annu. Rev. Biochem., 2001:503-33). The functional consequence of ubiquitination is determined by the number and linkage topology of ubiquitin molecules conjugated to the target protein. For example, proteins exhibiting Lys48-linked polyubiquitin chains are generally targeted to the proteasome for degradation, while monoubiquitination or polyubiquitin chains linked through other lysines regulate several non-proteolytic functions, including cell cycle regulation (Nakayama, K. I. et al., Nat. rev. Cancer, 6(5): 369-81 (2006)), DNA repair (Bergink, S., et al., Nature 458(7237): 461-7 (2009)), transcription (Conaway, R. C, et al., Science 296(5571): 1254-8 (2002)), and endocytosis (Mukhopadhyay, D., et al., Science 315(5809): 201-5 (2007)). Similar to other posttranslational modifications, ubiquitination is a reversible process counteracted by a family of enzymes known as deubiquitinases (DUBs). These enzymes are cysteine proteases or metalloproteases that hydrolyze the ubiquitin isopeptide bond (Komander, D., et al., Nat. Rev. Mol. Cell Biol. 10(8): 550-63 (2007)). The human genome encodes close to 100 DUBs.

DUBs and their substrate proteins are often deregulated in cancers. Targeting specific DUB family members may result in antitumor activity by enhancing the ubiquitination and subsequent degradation of oncogenic substrates, involved in tumor growth, survival, differentiation and maintenance of the tumor microenvironment. (Hussain, S. et, al., “DUBs and cancer: The role of deubiquitinating enzymes as oncogenes, non-oncogenes and tumor suppressors.” Cell Cycle 8, 1688-1697 (2009)). Consequently, several members of the DUB family have been implicated in processes related to human disease, including cancer and neurodegeneration. Among them, USP1 (ubiquitin-specific protease 1) has gained increased interest as a novel therapeutic target given its roles in the DNA damage response.

USP1 is a cysteine isopeptidase of the USP subfamily of deubiquitinases (DUBs). (Nijman, S. M. B., et al. “The deubiquitinating enzyme USP1 regulates the Fanconi anemia pathway. Mol. Cell 17, 331-339 (2005)) Full-length human USP1 is composed of 785 amino acids, including a catalytic triad composed of Cys90, His593 and Asp751. (Villamil, M. A., et al, “Serine phosphorylation is critical for the activation of ubiquitin-specific protease 1 and its interaction with WD40-repeat protein UAF1.” Biochem. 51, 9112-9113 (2012)). USP1 is relatively inactive on its own and full enzymatic activity is achieved only when bound in a heterodimeric complex with USP1 Associated Factor 1 (UAF1), which also binds to and regulates the activity of USP12 and USP46. (Cohn, M. A., et al, “A UAF1-Containing Multisubunit Protein Complex Regulates the Fanconi Anemia Pathway.” Mol. Cell 28, 786-797 (2007)).

USP1 deubiquitinates a variety of cellular targets involved in different processes related to cancer. For example, USP1 deubiquitinates PCNA (proliferating cell nuclear antigen), a key protein in translesion synthesis (TLS), and FANCI/FANCD2 (Fanconi anemia group complementation group D2), a key protein complex in the Fanconi anemia (FA) pathway. (Nijman, S. M. B. et al “The deubiquitinating enzyme USP1 regulates the Fanconi anemia pathway.” Mol Cell 17, 331-339 (2005); Huang, T. T. et al, “Regulation of monoubiquitinated PCNA by DUB autocleavage.” Nat. Cell Biol 8, 339-347 (2006)). These DNA damage response (DDR) pathways are essential for repair of DNA damage, including those induced by DNA cross-linking agents such as cisplatin, mitomycin C (MMC), diepoxybutane, ionizing radiation and ultraviolet radiation. In addition, USP1 promotes cancer cell stem maintenance by increasing inhibitor of protein binding (ID) protein stability. Thus, USP1 inhibition may antagonize cancer cell growth by inducing cell cycle arrest and decreasing cancer stem cell maintenance via a decrease in ID protein stability. (Williams, S. A. et al, “USP1 deubiquitinates ID proteins to preserve a mesenchymal stem cell program in osteosarcoma.” Cell 146: 918-30 (2011); Lee, J. K. et al, “USP1 targeting impedes GBM growth by inhibiting stem cell maintenance and radioresistance.” Neuro Oncol. 18: 37-47 (2016)).

The compounds GW7647 and Pimozide have been described as inactivators of USP1. However, both compounds are limited by potency and off-target pharmacology, in part because both of them have noticeable activity against unrelated targets. Another small molecule inhibitor of USP1, C527, which was reported by D'Andrea et al. in WO2011/137320, sensitizes cells to both the crosslinking agent, mitomycin C, and the topoisomerase 1 inhibitor, camptothecin. However, C527 shows low micromolar inhibition of related USPs as well as dissimilar DUBs (i.e., UCHL-1 and UCHL-3). Another small molecule USP1-UAF1 inhibitor (ML323) has been more recently disclosed (Dexheimer et al, J. Med. Chem. 2014, 57, 8099-8110; Liang et al, Nature Chem. Bio. 2015, 10, 298-304; U.S. Pat. No. 9,802,904 B2). Additional USP1 inhibitors have also been described in WO2017087837, WO2020132269, WO2020139988, and WO2021163530.

The foregoing shows that there exists an unmet need for new selective inhibitors of USP1, and in addition, that inhibition of USP1 with small molecule inhibitors has the potential to be a treatment for cancers and other disorders. For these reasons, there remains a considerable need for potent small molecule inhibitors of USP1.

### SUMMARY

In one embodiment, provided is a compound of Formula (I) or a pharmaceutically acceptable salt, hydrate, solvate, prodrug, stereoisomer, or tautomer thereof; wherein:

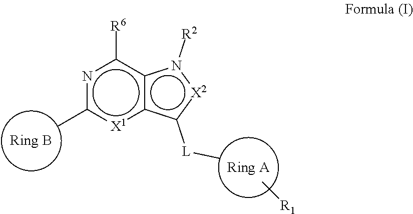

X¹ is selected from CH and N;
X² is selected from CR^{Xc2} and NR^{Xn2}
Ring B is a 5-6 member optionally substituted monocyclic aryl or heteroaryl.
L is selected from —O—, —NRⁿ—, —S—, —S(═O)—, —S(═O)₂— and —CR^{c}R^{c′}—;
Ring A is selected from C₆-C₁₀ aryl, 5-10 membered heteroaryl and 3-10 membered heterocyclyl, substituted with 0, 1 or 2 instances of halo or -Me;
R¹ is an optionally substituted 5-10 membered heteroaryl;
R² is absent or is selected from H, —C₁-C₆ alkyl, —C₃-C₉ cycloalkyl, —C₁-C₆ haloalkyl, —C₁-C₆ heteroalkyl, —C₁-C₆ hydroxyalkyl, arylalkyl, —S(═O)₂C₁-C₆alkyl and —C(═O)C₁-C₆alkyl, wherein each hydrogen of the alkyl, haloalkyl, heteroalkyl, hydroxylalkyl and arylalkyl can be independently replaced with a deuterium atom;
R⁶ is selected from H, -D, —CN, halo, —C₁-C₆ alkyl, —C₁-C₆ heteroalkyl, —C₁-C₆ haloalkyl, —C₃-C₁₀ cycloalkyl, —OH, and —O(C₁-C₆ alkyl);
each R^{Xc2} is independently selected from H, -D, halo, —C₁-C₆ alkyl, —C₁-C₆ heteroalkyl, —NH₂, —NH(C₁-C₆ alkyl), —O(C₁-C₆ alkyl) and —N(C₁-C₆ alkyl)₂;
each R^{Xn1} is absent or independently selected from H, —C₁-C₆ alkyl —C₁-C₆ haloalkyl, —S(═O)₂C₁-C₆alkyl and —C(═O)C₁-C₆alkyl;
each Rⁿ is independently selected from H and —C₁-C₆ alkyl; and
each R^{c} and R^{c′} is independently selected from H, —C₁-C₆ alkyl, —C₁-C₆ heteroalkyl, —C₁-C₆ haloalkyl, —OH, and —O(C₁-C₆ alkyl), or R^{c} and R^{c′} can be taken together with the atom to which they are attached to form a —C₃-C₉ cycloalkyl or a carbonyl.

In some embodiments, Ring B is substituted with 0, 1, 2 or 3 instances of R^{b}, wherein each R^{b} is independently selected from D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{b1}, —N(R^{b1})₂, —C(═O)R^{b1}, —C(═O)OR^{b1}, —NR^{b1}C(═O)R^{b1}, —NR^{b1}C(═O)OR^{b1}, —C(═O)N(R^{b1})₂, —OC(═O)N(R^{b1})₂, —S(═O)R^{b1}, —S(═O)₂R^{b1}, —SR^{b1}, —S(═O)(═NR^{b1})R^{b1}, —NR^{b1}S(═O)₂R^{b1} and —S(═O)₂N(R^{b1})₂ wherein each alkyl, cycloalkyl, heterocyclyl, aryl, heteroaryl, arylalkyl and heteroarylalkyl is optionally substituted at any available position;
each R^{b1} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl, wherein each hydrogen of the —C₁-C₆ alkyl of R^{b1} can be independently replaced with a deuterium atom.

In some embodiments, provided is a compound of Formula (II) or a pharmaceutically acceptable salt, hydrate, solvate, prodrug, stereoisomer, or tautomer thereof; wherein:

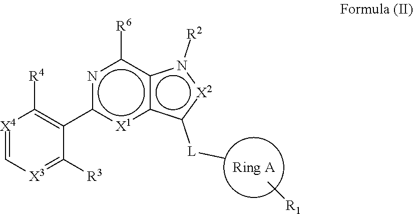

X¹ is selected from CH and N;
X² is selected from CR^{Xc2} and NR^{Xn2}
X³ is selected from CH and N;
X⁴ is selected from CH and N;
L is selected from —O—, —NRⁿ—, —S—, —S(═O)—, —S(═O)₂— and —CR^{c}R^{c′}—;
Ring A is selected from C₆-C₁₀ aryl, 5-10 membered heteroaryl and 3-10 membered heterocyclyl, each substituted with 0, 1 or 2 instances of halo or -Me;
R¹ is an optionally substituted 5-10 membered heteroaryl;
R² is absent or is selected from H, —C₁-C₆ alkyl, —C₃-C₉ cycloalkyl, —C₁-C₆ heteroalkyl, —C₁-C₆ haloalkyl, —C₁-C₆ hydroxyalkyl, arylalkyl, —S(═O)₂C₁-C₆ alkyl and —C(═O)C₁-C₆ alkyl, wherein each hydrogen of the alkyl, haloalkyl, heteroalkyl, hydroxylalkyl and arylalkyl can be independently replaced with a deuterium atom;
R³ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R³, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position.
R⁴ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position;
each R^{Xc2} is independently selected from H, —C₁-C₆ alkyl, —C₁-C₆ heteroalkyl, —NH₂, —NH(C₁-C₆ alkyl) and —N(C₁-C₆ alkyl)₂;
each R^{Xn2} is absent or independently selected from H, —C₁-C₆ alkyl, —C₁-C₆ haloalkyl, —S(═O)₂C₁-C₆alkyl and —C(═O)C₁-C₆ alkyl;
each Rⁿ is independently selected from H and —C₁-C₆ alkyl;
each R^{c} and R^{c′} is independently selected from H, —C₁-C₆ alkyl, —C₁-C₆ heteroalkyl, —C₁-C₆ haloalkyl, —OH, and —O(C₁-C₆ alkyl), or R^{c} and R^{c′} can be taken together with the atom to which they are attached to form a —C₃-C₉ cycloalkyl or a carbonyl; and
each R^{a3} and R^{a4} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each hydrogen of the —C₁-C₆ alkyl can be independently replaced with a deuterium atom.

In some embodiments, provided are compounds selected from the compounds of Table 1, or a pharmaceutically acceptable salt, hydrate, solvate, prodrug, stereoisomer, or tautomer thereof.

In some embodiments, provided is a pharmaceutical composition comprising a compound as described herein or a pharmaceutically acceptable salt, hydrate, solvate, prodrug, stereoisomer, or tautomer thereof and a pharmaceutically acceptable carrier.

In some embodiments, the pharmaceutical composition comprises a second therapeutic agent.

In some embodiments, provided is a method for treating or preventing a disease or disorder associated with the inhibition of USP1 comprising administering to a patient in need thereof an effective amount of a compound of Formula (I) as described herein or a pharmaceutically acceptable salt, hydrate, solvate, prodrug, stereoisomer, or tautomer thereof.

In some embodiments, provided is a method of treating a disease or disorder associated with the inhibition of USP1 comprising administering to a patient in need thereof an effective amount of a compound of Formula (I) as described herein or a pharmaceutically acceptable salt, hydrate, solvate, prodrug, stereoisomer, or tautomer thereof.

In some embodiments, provided is a method for inhibiting USP1 comprising administering to a patient in need thereof an effective amount of a compound of Formula (I) as described herein or a pharmaceutically acceptable salt, hydrate, solvate, prodrug, stereoisomer, or tautomer thereof.

In some embodiments, provided is a method for treating or preventing cancer in a patient in need thereof comprising administering to the patient in need thereof an effective amount of a compound of Formula (I) as described herein or a pharmaceutically acceptable salt, hydrate, solvate, prodrug, stereoisomer, or tautomer thereof.

In some embodiments, provided is a method for treating cancer in a patient in need thereof comprising administering to the patient in need thereof an effective amount of a compound of Formula (I) as described herein or a pharmaceutically acceptable salt, hydrate, solvate, prodrug, stereoisomer, or tautomer thereof.

In some embodiments, provided is a method for treating or preventing a disease or disorder associated with DNA damage comprising administering to a patient in need of a treatment for diseases or disorders associated with DNA damage an effective amount of a compound of Formula (I) as described herein or a pharmaceutically acceptable salt, hydrate, solvate, prodrug, stereoisomer, or tautomer thereof. In some embodiments the disease is cancer.

In some embodiments, provided is a method for treating a disease or disorder associated with DNA damage comprising administering to a patient in need of a treatment for diseases or disorders associated with DNA damage an effective amount (e.g., a therapeutically effective amount) of a compound of Formula (I) as described herein or a pharmaceutically acceptable salt, hydrate, solvate, prodrug, stereoisomer, or tautomer thereof.

In some embodiments, provided is a method of inhibiting, modulating or reducing DNA repair activity exercised by USP1 comprising administering to a patient in need thereof an effective amount of a compound of Formula (I) as described herein or a pharmaceutically acceptable salt, hydrate, solvate, prodrug, stereoisomer, or tautomer thereof.

### DETAILED DESCRIPTION

The disclosure herein sets forth exemplary methods, parameters and the like. It should be recognized, however, that such description is not intended as a limitation on the scope of the present disclosure but is instead provided as a description of exemplary embodiments.

### Definitions

As used in the present disclosure, the following words and phrases are generally intended to have the meanings as set forth below unless expressly indicated otherwise or the context in which they are used indicates otherwise.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example. Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ, of Notre Dame Press, Notre Dame, IN 1972). The invention additionally encompasses compounds described herein as individual isomers substantially free of other isomers, and alternatively, as mixtures of various isomers.

The “enantiomeric excess” (“e.e.”) or “1% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

e.e.=(90−10)/100=80%.

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

d.e.=(90−10)/100=80%.

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹³N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃.

“Aliphatic” refers to an alkyl, alkenyl, alkynyl, or carbocyclyl group, as defined herein.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Aralkyl” or “arylalkyl” is a subset of alkyl and aryl, as defined herein, and refers to an optionally substituted alkyl group substituted by an optionally substituted aryl group.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), ⁱPr (—CH(CH₃)₂), ⁿPr (—CH₂CH₂CH₃), ⁿBu (—CH₂CH₂CH₂CH₃), or ^{t}Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₂CH₂CH₂—, —CH₂C(CH₃)₂CH₂—, —CH₂CH₂C(CH₃)₂—), and the like.

When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“(C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

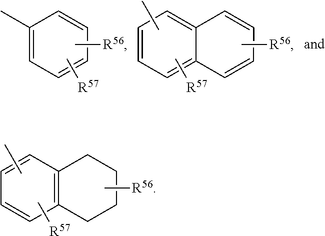

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONROR, CONR⁵⁸OR⁵⁹, N⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In some embodiments, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In some embodiments, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In some embodiments, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepanyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

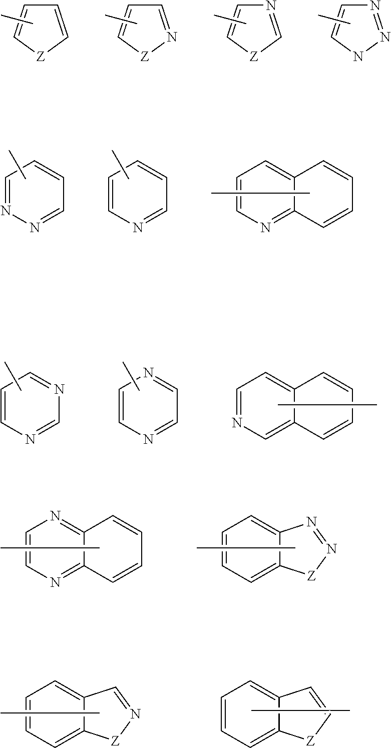

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic) cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group, wherein the point of attachment is on the alkyl moiety.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system.

Carbocyclyl groups include fully saturated ring systems (e.g., cycloalkyls), and partially saturated ring systems. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2,2,2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄ tricyclic, or C₆-C₁₄ polycyclic cycloalkyl). In some embodiments “cycloalkyl” is a monocyclic cycloalkyl. In some embodiments, a monocyclic cycloalkyl has 3-14 ring carbon atoms. (“C₃₋₁₄ monocyclic cycloalkyl”). In some embodiments, a monocyclic cycloalkyl group has 3 to 10 ring carbon atoms (“C₃₋₁₀ monocyclic cycloalkyl”). In some embodiments, a monocyclic cycloalkyl group has 3 to 8 ring carbon atoms (“C₃₋₈ monocyclic cycloalkyl”). In some embodiments, a monocyclic cycloalkyl group has 3 to 6 ring carbon atoms (“C₃₋₆ monocyclic cycloalkyl”). In some embodiments, a monocyclic cycloalkyl group has 4 to 6 ring carbon atoms (“C₄₋₆ monocyclic cycloalkyl”). In some embodiments, a monocyclic cycloalkyl group has 5 to 6 ring carbon atoms (“C₅₋₆ monocyclic cycloalkyl”). In some embodiments, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C-s cycloalkyl groups include the aforementioned C₃₋₆ cycloalkyl groups as well as cycloheptyl (C₇) and cyclooctyl (C₈).

In some embodiments “cycloalkyl” is a bicyclic cycloalkyl. In some embodiments, a bicyclic cycloalkyl has 4-14 ring carbon atoms. (“C₄₋₁₄ bicyclic cycloalkyl”). In some embodiments, a bicyclic cycloalkyl group has 4 to 12 ring carbon atoms (“C₄₋₁₂ bicyclic cycloalkyl”). In some embodiments, a bicyclic cycloalkyl group has 4 to 10 ring carbon atoms (“C₄₋₁₀ bicyclic cycloalkyl”). In some embodiments, a bicyclic cycloalkyl group has 5 to 10 ring carbon atoms (“C₅₋₁₀ bicyclic cycloalkyl”). In some embodiments, a bicyclic cycloalkyl group has 6 to 10 ring carbon atoms (“C₆₋₁₀ bicyclic cycloalkyl”). In some embodiments, a bicyclic cycloalkyl group has 8 to 10 ring carbon atoms (“C₈₋₁₀ bicyclic cycloalkyl”). In some embodiments, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₅), spiro[2.2] pentane (C₅), bicyclo[2.1.0]pentane (C₅), bicyclo[2.1.1]hexane (C₆), bicyclo[3.1.0]hexane (C₆), spiro[2.3] hexane (C₆), bicyclo[2.2.1]heptane (norhopane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4] heptane (C₇), spiro[3.3]heptane (C₇), bicyclo[2,2,2]octane (C₈), bicyclo[4.1.1]octane (C₈)octahydropentalene (C₈), bicyclo[3.2.1]octane (C₈), bicyclo[4.2.0]octane (C₈), spiro[2.5]octane (C₈), spiro[3.4]octane (C₈), bicyclo[3.3.1]nonane (C₉), octahydro-1H-indene (C₉), bicyclo[4.2.1]nonane (C₉), spiro[3,5]nonane (C₉), spiro[4.4]nonane (C₉), bicyclo[3.3.2]decane (C₁₀), bicyclo[4,3,1]decane (C₁₀), spiro[4.5]decane (C₁₀), bicyclo[3.3.3]undecane (C₁₀), decahydronaphthalene (C₁₀), bicyclo[4,3,2]undecane (C₁₁), spiro[5.5]undecane (C₁₁) and bicyclo[4,3,3]dodecane (C₁₂).

In some embodiments “cycloalkyl” is a tricyclic cycloalkyl. In some embodiments, a tricyclic cycloalkyl has 6-14 ring carbon atoms. (“C₆₋₁₄ tricyclic cycloalkyl”). In some embodiments, a tricyclic cycloalkyl group has 8 to 12 ring carbon atoms (“C₈₋₁₂ tricyclic cycloalkyl”). In some embodiments, a tricyclic cycloalkyl group has 10 to 12 ring carbon atoms (“C₁₀₋₁₂ tricyclic cycloalkyl. Examples of tricyclic cycloalkyls include adamantine (C₁₂). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzo-thienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(═O)—C₁-C₈ alkyl, —C(═O)—(CH₂)(C₆-C₁₀ aryl), —C(═O)—(CH₂)ₜ(5-10 membered heteroaryl), —C(═O)—(CH₂)N(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals respectively. In some embodiments the alkylamino is a —NH(C₁-C₄ alkyl). In some embodiments the alkylamino is methylamino, ethylamino, propylamino, isopropylamino, n-butylamino, iso-butylamino, sec-butylamino or tert-butylamino. In some embodiments the dialkylamino is —N(C₁-C₆ alkyl)₂. In some embodiments the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In some embodiments the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In some embodiments haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)ₜ— and aryl-S(O)₂—. Exemplary ‘substituted alkoxy’ groups include, but are not limited to, —O—(CH₂)ₜC₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Oxo group” refers to —C(═O)—.

“Substituted amino” refers to an amino group of the formula —N(R³⁸)₂ wherein R³ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)(5-10 membered heteroaryl), —(CH₂(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R¹ groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion.

each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —COH, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂₀R^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Ru groups;
each instance of R^{ff} is, independently, selected from hydrogen, —C₁₋₄ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl. C₃₋₁₀ carbocyclyl, 3-10 membered heterocyclyl, C₆₋₁₀ aryl and 5-10 membered heteroaryl, or two R groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 RU groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₃, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-r-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂₁R) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative. N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, I-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentacblorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, l-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4.4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile.

Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such asp-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (C₁), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R_{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₃, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂₀R^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{d} groups;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{d} substituents can be joined to form ═O or ═S:
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R⁹ groups;
each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₄ alkyl)₂⁺X⁻, —NH₂(C₁₋₆ alkyl)⁺X⁻, —NH₃⁺X⁻, —N(OC₁₋₆ alkyl)(C₁₋₆alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₄ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻2sulfonate ions (e.g., methansulfonate, trifluoromethanesulfonate, p-toluencsulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate.glycerate, lactate, tartrate.glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

The term “about,” as used herein, includes the recited number±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

“USP1” and “ubiquitin-specific-processing protease 1” as used herein refer to any native polypeptide or USP1-encoding polynucleotide. The term “USP1“encompasses” full-length,” unprocessed USP1 polypeptide as well as any forms of USP1 that result from processing within the cell (e g., removal of the signal peptide). The term also encompasses naturally occurring variants of USP1, e.g., those encoded by splice variants and allelic variants. The USP1 polypeptides described herein can be isolated from a variety of sources, such as from human tissue types or from another source, or prepared by recombinant or synthetic methods. Human USP1 sequences are known and include, for example, the sequences publicly available as UniProt No. 094782 (including isoforms). As used herein, the term “human USP1 protein” refers to USP1 protein comprising the amino acid sequence as set forth in SEQ ID NO: 1 in U S. provisional patent application No. 62/857,986 filed Jun. 6, 2019.

USP1 is a deubiquitinating enzyme that acts as part of a complex with UAF1. USP1's “deubiquitinase activity” includes its ability to deubiquitinate as part of the USP1-UAF1 complex.

The term “specifically binds” to a protein or domain of a protein is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular protein or domain of a protein than it does with alternative proteins or domains. It should be understood that a molecule that specifically or preferentially binds to a first protein or domain may or may not specifically or preferentially bind to a second protein or domain. As such, “specific binding” or “preferential binding” does not necessarily require (although it can include) exclusive binding. Generally, but not necessarily, reference to binding means preferential binding. For example, a USP1 inhibitor that specifically binds to USP1, UAF1, and/or the USP1-UAF1 complex may not bind to other deubiquitinases, other USP proteins, or other UAF1 complexes (e.g., USP46-UAF1) or may bind to other deubiquitinases, other USP proteins, or other UAF1 complexes (e.g., USP46-UAF1) with a reduced affinity as compared to binding to USP1.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

In some embodiments inhibiting USP1 proteins is the inhibition of one or more activities or functions of USP1 proteins. It should be appreciated that the activity or function of the one or more USP1 proteins may be inhibited in vitro or in vivo. Non limiting examples of activities and functions of USP1 include deubiquitinase activity, and formation of a complex with UAF1 and are described herein. Examplary levels of inhibition of the activity of one or more USP1 proteins include at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

As used herein, the term “loss of function” mutation refers to a mutation that that results in the absence of a gene, decreased expression of a gene, or the production of a gene product (e.g., protein) having decreased activity or no activity. Loss of function mutations include for example, missense mutations, nucleotide insertions, nucleotide deletions, and gene deletions.

Loss of function mutations also include dominant negative mutations. Thus, cancer cells with a loss of function mutation in a gene encoding BRCA1 include cancer cells that contain missense mutations in a gene encoding BRCA1 as well as cancer cells that lack a gene encoding BRCA1.

As used herein, the term “salt” refers to any and all salts and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate.glucoheptonate.glycerophosphate.gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts.

Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomologus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In some embodiments, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response. As will be appreciated by those of ordinary skill in this art, the effective amount of a compound disclosed herein may vary depending on such factors as the desired biological endpoint, the pharmacokinetics of the compound, the disease being treated, the mode of administration, and the age, health, and condition of the subject. An effective amount encompasses therapeutic and prophylactic treatment. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

The term “container” means any receptacle and closure therefore suitable for storing, shipping, dispensing, and/or handling a pharmaceutical product.

The term “insert” or “package insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

### Compounds

As generally described herein, provided are compounds (e.g., compounds of Formula (I), (II), (IIa), (IIa1), (IIb), (IIb1), (IIc), (IIc1), (IIc′), (IIc1′) or a compound of Table 1 or pharmaceutically acceptable salts, hydrates, solvates, prodrugs, stereoisomers or tautomers thereof) that are ubiquitin-specific-processing protease 1 (USP1) inhibitors useful for treating diseases and disorders (e.g., cancers) associated with USP1.

Provided herein are compounds of Formula (I). Unless the context requires otherwise, reference throughout this specification to “a compound of Formula (I)” or “compounds of Formula (I)” refers to all embodiments of Formula (I), including, for example, compounds of (I), (II), (IIa), (IIa1), (IIb), (IIb1), (IIc), (IIc1), (IIc′), (IIc1′) (i.e., Formula (I)-Formula (IIc1′) as well as the compounds of Table 1.

In some embodiments, provided are compounds of Formula (I) or a pharmaceutically acceptable salt, hydrate, solvate, prodrug, stereoisomer, or tautomer thereof. In some embodiments, including any of the numbered embodiments described herein, the compounds are provided as free base or pharmaceutically acceptable salts. In some embodiments, including any of the numbered embodiments described herein, the compounds are provided as free base. In some embodiments, including any of the numbered embodiments described herein, the compounds are provided as pharmaceutically acceptable salts.

In one embodiment, provided is a compound of Formula (I) or a pharmaceutically acceptable salt, hydrate, solvate, prodrug, stereoisomer, or tautomer thereof; wherein:

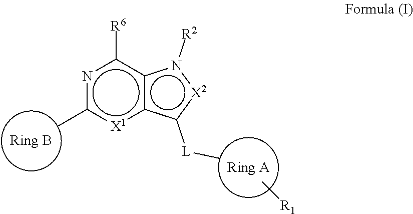

X¹ is selected from CH and N;
X¹ is selected from CR^{Xc2} and NR^{Xn2}
Ring B is a 5-6 member optionally substituted monocyclic aryl or heteroaryl.
L is selected from —O—, —NRⁿ—, —S—, —S(═O)—, —S(═O)₂— and —CR^{c}R^{c′};
Ring A is selected from C₆-C₁₀ aryl, 5-10 membered heteroaryl and 3-10 membered heterocyclyl, substituted with 0, 1 or 2 instances of halo or -Me;
R¹ is an optionally substituted 5-10 membered heteroaryl;
R² is absent or is selected from H, —C₁-C₆ alkyl, —C₃-C₉ cycloalkyl (e.g., cyclopropyl), —C₁-C₆ haloalkyl, —C₁-C₆ heteroalkyl, —C₁-C₆ hydroxyalkyl, arylalkyl, —S(═O)₂C₁-C₆alkyl (e.g., —S(═O)₂CH₃) and —C(═O)C₁-C₆ alkyl (e.g., —C(═O)CH₃), wherein each hydrogen of the alkyl, haloalkyl, heteroalkyl, hydroxylalkyl and arylalkyl can be independently replaced with a deuterium atom;
R⁶ is selected from H, -D, —CN halo, —C₁-C₆ alkyl, —C₁-C₆ heteroalkyl, —C₁-C₆ haloalkyl, —C₃-C₁₀ cycloalkyl, —OH, and —O(C₁-C₆ alkyl);
each R^{Xc2} is independently selected from H, -D, halo, —C₁-C₆ alkyl, —C₁-C₆ heteroalkyl, —NH₂—, —NH(C₁-C₆ alkyl), —O(C₁-C₆ alkyl) and —N(C₁-C₆ alkyl)₂;
each R^{Xn2} is absent or independently selected from H, —C₁-C₆ alkyl —C₁-C₆ haloalkyl (e.g., CH₂CF₃), —S(═O)₂C₁-C₆alkyl (e.g., —S(═O)₂CH₃) and —C(═O)C₁-C₆alkyl (e.g., —C(═O)CH₃);
each Rⁿ is independently selected from H and —C₁-C₆ alkyl (e.g., -Me); and
each R^{c} and R^{c′} is independently selected from H, —C₁-C₆ alkyl (e.g., Me), —C₁-C₆ heteroalkyl, —C₁-C₆ haloalkyl, —OH, and —O(C₁-C₆ alkyl) (e.g., —OMe), or R^{c} and R^{c′} can be taken together with the atom to which they are attached to form a —C₃-C₉ cycloalkyl (e.g., cyclopropyl) or a carbonyl.

As generally defined herein, Ring B is a 5-6 member optionally substituted monocyclic aryl or heteroaryl. In some embodiments, Ring B is substituted with 0, 1, 2 or 3 instances of R^{b}.

In some embodiments, Ring B is substituted with 0, 1 or 2 instances of R^{b}. In some embodiments, Ring B is substituted with 1 or 2 instances of R^{b}. In some embodiments, Ring B is substituted with 1 instance of R^{b}. In some embodiments, Ring B is substituted with 2 instances of R^{b}.

In certain embodiments, Ring B is an optionally substituted 5-membered heteroaryl containing 1-3 heteroatoms independently selected from O, N and S. In some embodiments, Ring B is a 5-membered heteroaryl containing 1-3 heteroatoms independently selected from O, N and S, substituted with 0, 1, 2 or 3 instances of R^{b}. In further embodiments, Ring B is a 5-membered heteroaryl ring selected from pyrrolyl, thiophenyl, furyl, pyrazolyl, imidazolyl, thiazolyl, oxazolyl, isoxazolyl, isothiazolyl, triazolyl, tetrazolyl, oxadiazolyl and thiadiazolyl, each of which can be optionally substituted (e.g., substituted with 0, 1, 2 or 3 instances of R^{b}).

In some embodiments, Ring B is pyrazolyl (e.g., pyrazol-2-yl) substituted with 0, 1 or 2 instances of methyl, trifluoromethyl, or a combination thereof.

In certain embodiments, Ring B is an optionally substituted 6 membered heteroaryl containing 1-3 nitrogen atoms. In certain embodiments, Ring B is a 6 membered heteroaryl containing 1-3 nitrogen atoms, substituted with 0, 1, 2 or 3 instances of R^{b}.

In some embodiments, Ring B is selected from pyridinyl, pyrimidinyl, pyrazinyl, triazinyl and pyridazinyl, each of which can be optionally substituted (e.g., substituted with 0, 1, 2 or 3 instances of R^{b}). In some embodiments, Ring B is selected from pyridinyl and pyrimidinyl, each of which can be optionally substituted (e.g., substituted with 0, 1, 2 or 3 instances of R^{b}).

In certain embodiments, Ring B is selected from pyrazolyl, phenyl, pyridinyl and pyrimidinyl, each of which can be optionally substituted (e.g., substituted with 0, 1, 2 or 3 instances of R^{b}). In further embodiments, Ring B is optionally substituted phenyl (e.g., substituted with 0, 1, 2 or 3 instances of R^{b}).

In certain embodiments, Ring B is selected from phenyl, pyridinyl and pyrimidinyl, each of which can be optionally substituted (e.g., substituted with 0, 1, 2 or 3 instances of R^{b}). In further embodiments, Ring B is optionally substituted phenyl (e.g., substituted with 0, 1, 2 or 3 instances of R^{b}).

As generally defined herein, each R^{b} is independently selected from D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{b1}, —N(R^{b1})₂, —C(═O)R^{b1}, —C(═O)OR^{b1}, —NR^{b1}C(═O)R^{b1}, —NR^{b1}C(═O)OR^{b1}, —C(═O)N(R^{b1}), —OC(═O)N(R^{b1})₂, —S(═O)R^{b1}, —S(═O)₂R^{b1}, —SR^{b1}, —S(═O)(═NR^{b1})R^{b1}, —NR^{b1}S(═O)₂R^{b1} and —S(═O)₂N(R^{b1})₂ wherein each alkyl, cycloalkyl, heterocyclyl, aryl, heteroaryl, arylalkyl and heteroarylalkyl is optionally substituted at any available position.

In certain embodiments, each R^{b} is independently selected from halo, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{b1} and —N(R^{b1})₂, wherein each alkyl, cycloalkyl and heterocyclyl is substituted with 0, 1, 2 or 3 instances of halo (e.g., —F, Cl), —OH, CN, -Me, -Et, —NH₂ or oxo and wherein each R^{b1} is independently selected from H, —C₁-C₆ alkyl, —C₁-C₆ heteroalkyl, —C₁-C₆ haloalkyl and C₃-C₉ cycloalkyl wherein each hydrogen of the —C₁-C₆ alkyl of R^{b1} can be independently replaced with a deuterium atom.

In certain embodiments, each R^{b} is independently selected from halo, —C₁-C₆ alkyl (e.g., -Me, -Et, —Pr, -ⁱPr, -ⁿBu, -sec-Bu, -iso-Bu, -^{t}Bu), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃)—C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrolidinyl, piperidinyl, piperazinyl, morpholinyl, 6-oxa-1-azaspiro[3.3]heptanyl, 6-oxa-1-azaspiro[3.4]octanyl), —OR^{b1} and —N(R^{b1})₂, wherein each alkyl, cycloalkyl and heterocyclyl is substituted with 0, 1, 2 or 3 instances of halo (e.g., —F, Cl), and wherein each R^{b1} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CH₂F, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl), wherein each hydrogen of the —C₁-C₆ alkyl of R^{b1} can be independently replaced with a deuterium atom.

In some embodiments, each R^{b} is independently selected from —Cl, -ⁱPr, —CH₂N(CH₃)CH₂CH₃, —CH₂N(CH₃)₂, —CF₃, —CH₂OH, —CH(OH)(CH)₂, cyclopropyl (substituted with 0, 1 or 2 instances of —F), azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OCH₂F, —OⁱPr, —OPr, —OMe, —OCD₃, —OEt, —OH, —Ocyclopropyl, —N(Me)₂, —NHMe and —NHⁱPr.

In some embodiments R^{b} is -D.

In certain embodiments, R^{b} is halo (e.g., fluoro, chloro, bromo, iodo). In further embodiments, R^{b} is —C₁. In some embodiments, R^{b} is —F. In some embodiments, R^{b} is —Br. In some embodiments, R^{b} is —I.

In some embodiments, R^{b} is —CN.

In certain embodiments, R^{b} is —C₁-C₆ alkyl (e.g., -Me, -Et, —Pr, —ⁱPr, -ⁿBu, -sec-Bu, -iso-Bu, -^{t}Bu). In further embodiments, R^{b} is -Me. In some embodiments, R^{b} is -Et. In some embodiments R^{b} is —Pr. In some embodiments, R^{b} is -iPr.

In some embodiments, R^{b} is —C₁-C₆ heteroalkyl. In further embodiments, R^{b} is methoxymethyl (—CH₂OCH₃). In some embodiments, R^{b} is hydroxymethyl (—CH₂OH). In some embodiments, R^{b} is aminomethyl (e.g., —CH₂NH₂, —CH₂NHCH₃, —CH₂N(CH₃)₂. In some embodiments, R^{b} is —CH₂N(CH₃)CH₂CH₃.

In some embodiments, R^{b} is —C₁-C₆ haloalkyl. In further embodiments, R^{b} is trifluoromethyl (—CF₃). In other embodiments, R^{b} is difluoromethyl (—CHF₂).

In some embodiments, R^{b} is —C₁-C₆ hydroxyalkyl (e.g., —CH₂OH, —CH₂CH₂OH).

In some embodiments, R^{b} is —C₃-C₁₀ cycloalkyl (e.g., cyclopropyl, cyclobutyl, cyclopentyl, cyclohexyl). In some embodiments, R^{b} is cyclopropyl. In some embodiments R^{b} is cyclobutyl. In some embodiments, R^{b} is cyclopentyl. In some embodiments, R^{b} is cyclohexyl.

In some embodiments, R^{b} is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3.3]heptanyl, 6-oxa-1-azaspiro[3.4]octanyl). In some embodiments, R^{b} is oxetanyl. In some embodiments, R^{b} is tetrahydropyranyl. In some embodiments, R^{b} is tetrahydrofuranyl. In some embodiments, R^{b} is azetidinyl. In some embodiments, R^{b} is pyrrolidinyl. In some embodiments, R^{b} is piperidinyl. In some embodiments, R^{b} is piperazinyl. In some embodiments, R^{b} is morpholinyl. In some embodiments, R^{b} is azepanyl. In some embodiments, R^{b} is 6-oxa-1-azaspiro[3.3]heptanyl. In some embodiments, R⁶ is 6-oxa-1-azaspiro[3.4]octanyl.

In some embodiments R^{b} is cycloalkylalkyl (e.g., cyclopropylmethyl, cyclobutylmethyl, cyclopentylmethyl, cyclohexylmethyl, cycloheptylmethyl). In some embodiments, R^{b} is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R^{b} is arylalkyl. In some embodiments, R^{b} is benzyl.

In some embodiments, R^{b} is heteroarylalkyl (e.g., pyridinylmethyl, thiazolylmethyl, triazolylmethyl, pyrazolylmethyl).

In some embodiments, R^{b} is —OR^{b1} (e.g., hydroxy (—OH), methoxy, difluoromethoxy (—OCHF₂), trifluoromethoxy (—OCF₃), —OCH(CH₃)CF₃, —OCH₂CF₃, ethoxy, propoxy, isopropoxy, cyclopropyloxy, cyclobutyloxy,). In some embodiments, R^{b} is hydroxy. In some embodiments, R^{b} is methoxy. In some embodiments, R^{b} is ethoxy. In some embodiments, R^{b} is propoxy. In some embodiments, R^{b} is isopropoxy. In some embodiments R^{b} is difluoromethoxy. (—OCHF₂). In some embodiments, R^{b} is trifluoromethoxy (—OCF₃). In some embodiments, R^{b} is —OCH(CH₃)CF₃. In some embodiments, R^{b} is —OCH₂CF₃. In some embodiments, R^{b} is cyclopropyloxy.

In some embodiments, R^{b} is —N(R^{b1})₂ (e.g., —NH₂, —NHR^{b1}, —N(CH₃)R^{b1}). In some embodiments, R^{b} is —NH₂. In some embodiments, R^{b} is —NHR^{b1} (e.g., —NHMe, —NHEt, —NHPr, —NHⁱPr, —NHcyclopropyl, —NHcyclobutyl). In some embodiments, R^{b} is —N(CH₃)R^{b1} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R^{b} is —C(═O)R^{b1} or —C(═O)OR^{b1}. In some embodiments, R^{b} is —C(═O)R^{b1} wherein R^{b1} is as described herein. In some embodiments, R^{b} is —C(═O)alkyl. In some embodiments, R^{b} is —C(O)CH₃, —C(O)cyclopropyl, —C(O)cyclobutyl, —C(O)Bu, —C(O)ⁱPr, —C(O)Pr, —C(O)^{t}Bu, or —C(═O)OMe. In some embodiments, R^{b} is acetyl (—C(═O)Me). In some embodiments, R^{b} is —C(═O)ORI. In some embodiments, R^{b} is —COOH. In some embodiments, R^{b} is COOMe.

In some embodiments, R^{b} is —NR^{b1}C(═O)R^{b1}. In certain embodiments, R^{b} is —NHC(═O)R^{b1} (e.g., NHC(═O)Me, NHC(═O)Et, NHC(═O)Pr, NHC(═O)ⁱPr, NHC(═O)Bu, NHC(═O)^{t}Bu, NHC(═O)Cyclopropyl, NHC(═O)Cyclobutyl). In some embodiments, R^{b} is —N(CH₃)C(═O)R^{b1} (e.g., N(CH₃)C(═O)Me, N(CH₃)C(═O)Et, N(CH₃)C(═O)Pr, N(CH₃)C(═O)ⁱPr, N(CH₃)C(═O)Bu, N(CH₃)C(═O)^{t}Bu, N(CH₃)C(═O)Cyclopropyl, N(CH₃)C(═O)Cyclobutyl).

In some embodiments, R^{b} is —NR^{b1}C(═O)OR^{b1}. In certain embodiments, R^{b} is —NHC(═O)OR^{b1} (e.g., NHC(═O)OMe, NHC(═O)OEt, NHC(═O)OPr, NHC(═O)OⁱPr, NHC(═O)OBu, NHC(═O)O^{t}Bu, NHC(═O)OCyclopropyl, NHC(═O)OCyclobutyl). In some embodiments, R^{b} is —N(CH₃)C(═O)OR^{b1} (e.g., N(CH₃)C(═O)OMe, N(CH₃)C(═O)OEt, N(CH₃)C(═O)OPr, N(CH₃)C(═O)OⁱPr, N(CH₃)C(O)OBu, N(CH₃)C(═O)O^{t}Bu, N(CH₃)C(═O)OCyclopropyl, N(CH₃)C(═O)OCyclobutyl).

In some embodiments, R^{b} is —C(═O)N(R^{b1})₂ (e.g., —C(═O)NH₂, —C(═O)NHR^{b1}, —C(═O)N(CH₃)R^{b1}). In some embodiments, R^{b} is —C(═O)NH₂. In certain embodiments, R^{b} is —C(═O)NHR^{b1} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R^{b} is —C(═O)N(CH₃)R^{b1} (e.g., —C(═O)NMe₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R^{b} is —OC(═O)N(R^{b1})₂. In certain embodiments, R^{b} is —OC(═O)NHR^{b1} (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R^{b} is —OC(═O)N(CH₃)R^{b1} (e.g., —OC(═O)NMe₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R^{b} is —S(═O)R^{b1}. In certain embodiments, R^{b} is —S(═O)alkyl (e.g., —S(═O)Me, —S(═O)Et, —S(═O)Pr, —S(═O)ⁱPr). In certain embodiments, R^{b} is —S(═O)cycloalkyl (e.g., —S(═O)cyclopropyl, —S(═O)cyclobutyl, —S(═O)cyclopentyl, —S(═O)cyclohexyl).

In some embodiments, R^{b} is —S(═O)₂R^{b1}. In certain embodiments, R^{b} is —S(═O)₂alkyl (e.g., —S(═O)₂Me, —S(═O)₂Et, —S(═O)₂Pr, —S(═O)₂ⁱPr). In certain embodiments, R^{b} is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R^{b} is S(═O)₂aryl (e.g., S(═O)₂phenyl).

In some embodiments, R^{b} is —SR^{b1}. In certain embodiments, R^{b} is —Salkyl (e.g., —SMe, —SEt, —SPr, —SⁱPr). In certain embodiments, R^{b} is —Scycloalkyl (e.g., —Scyclopropyl, —Scyclobutyl, —Scyclopentyl, —Scyclohexyl). In certain embodiments, R^{b} is —Saryl(e.g., Sphenyl).

In some embodiments, R^{b} is —S(═O)(═NR^{b1})R^{b1}. In certain embodiments, R^{b} is —S(═O)(═NH)R^{b1} (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)Bu, —S(═O)(═NH)Cyclopropyl, —S(═O)(═NH)Cyclobutyl). In some embodiments, R^{b} is —S(═O)(═NCH₃)R^{b1} (e.g., —S(═O)(═NCH₃)Me, —S(═O)(═NCH₃)Et, —S(═O)(═NCH₃)Pr, —S(═O)(═NCH₃)ⁱPr, —S(═O)(═NCH₃)Bu, —S(═O)(═NCH₃)Bu, —S(═O)(═NCH₃)Cyclopropyl, —S(═O)(═NCH₃)Cyclobutyl).

In some embodiments, R^{b} is —NR^{b}'S(═O)₂R^{b1}. In certain embodiments, R^{b} is —NHS(═O)₂alkyl (e.g., —NHS(═O)₂Me, —NHS(═O)₂Et, —NHS(═O)₂Pr, —NHS(═O)₂ⁱPr). In certain embodiments, R^{b} is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R^{b} is —N(CH₃)S(═O)₂alkyl (e.g., —N(CH₃)S(═O)₂Me, —N(CH)S(═O)₂Et, —N(CH₃)S(═O)₂Pr, —N(CH₃)S(═O)₂ⁱPr). In certain embodiments, R^{b} is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R^{b} is —S(═O)₂N(R^{b1})₂. (e.g., —S(═O)₂NH₂, —S(═O)₂NHR^{b1}, —S(═O)₂N(CH₃)R^{b1}). In some embodiments, R^{b} is —S(═O)₂NH₂. In some embodiments, R^{b} is —S(═O)₂NHR^{b1} (e.g., —S(═O)₂NHMe, —S(═O)₂NHEt, —S(═O)₂NHPr, —S(═O)₂NHⁱPr, —S(═O)₂NHcyclopropyl, —S(═O)₂NHcyclobutyl). In some embodiments, R^{b} is —S(═O)₂N(CH₃)R^{b1} (e.g., —S(═O)₂NMe₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R^{b1} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl, wherein each hydrogen of the —C₁-C₆ alkyl of R^{b1} can be independently replaced with a deuterium atom.

In some embodiments, each R^{b1} is independently selected from H, —C₁-C₆ alkyl (e.g., -Me, -Et, —Pr, -ⁱPr, -ⁿBu, -^{t}Bu, -sec-Bu, -iso-Bu) and —C₁-C₆ haloalkyl (e.g., —CHF₂, —CF₃, —CH(CH₃)CF₃, —CH₂CF₃) wherein each hydrogen of the —C₁-C₆ alkyl of R^{b1} can be independently replaced with a deuterium atom.

In some embodiments, each R^{b1} is independently H.

In some embodiments, each R^{b1} is independently —C₁-C₆ alkyl (e.g., -Me, -Et, —Pr, -ⁱPr, -ⁿBu, -^{t}Bu, -sec-Bu, -iso-Bu). In some embodiments, each R^{b1} is independently -Me. In some embodiments, each R^{b1} is independently -Et. In some embodiments, each R^{b1} is independently —Pr. In some embodiments, each R^{b1} is independently -ⁱPr. In some embodiments, one or more hydrogens of the —C₁-C₆ alkyl of R^{b1} is replaced with a deuterium atom. In some embodiments, R^{b1} is —CD₃.

In some embodiments, each R^{b1} is independently —C₁-C₆ heteroalkyl. In further embodiments, each R^{b1} is independently methoxymethyl (—CH₂OCH₃). In some embodiments, each R^{b1} is independently hydroxymethyl (—CH₂OH). In some embodiments, each R^{b1} is independently is aminomethyl (e.g., —CH₂NH₂, —CH₂NHCH₃, —CH₂N(CH₃)₂.

In some embodiments, each R^{b1} is independently —C₁-C₆ haloalkyl. In further embodiments, each R^{b1} is independently trifluoromethyl (—CF₃). In other embodiments, each R^{b1} is independently difluoromethyl (—CHF₂). In other embodiments, each R^{b1} is independently fluoromethyl (—CH₂F). In some embodiments, each R^{b1} is —CH(CH₃)CF₃. In some embodiments, each R^{b1} is —CH₂CF₃.

In some embodiments, each R^{b1} is independently —C₃-C₉ cycloalkyl (e.g., cyclopropyl, cyclobutyl, cyclopentyl, cyclohexyl). In some embodiments, each R^{b1} is independently cyclopropyl. In some embodiments each R^{b1} is independently cyclobutyl. In some embodiments, each R^{b1} is independently cyclopentyl. In some embodiments, each R^{b1} is independently cyclohexyl.

In some embodiments, each R^{b1} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{b1} is independently heteroaryl. In some embodiments, R^{b1} is independently a 5-10 member heteroaryl (e.g., a 5-6 member monocyclic heteroaryl or an 8-10 member bicyclic heteroaryl containing 1-3 heteroatoms independently selected from N, O and S). In some embodiments, R^{b1} is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{b1} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{b1} is independently thiophenyl (e.g., thiophen-2-yl, thiophen-3-yl). In some embodiments, R^{b} is independently pyrazolyl (e.g., pyrazol-1-yl, pyrazol-3-yl, pyrazol-5-yl). In some embodiments, R^{b1} is independently thiazolyl (e.g., thiazol-2-yl, thiazol-4-yl, thiazol-5-yl). In some embodiments, R^{b1} is independently a 6-member monocyclic heteroaryl (e.g., pyridyl, pyrimidinyl, triazinyl, pyrazinyl, pyridazinyl). In some embodiments, R^{b1} is independently pyridinyl (e.g., pyridin-2-yl, pyridin-3-yl, pyridin-4-yl). In some embodiments, R^{b1} is independently pyrimidinyl (e.g., pyrimidin-2-yl, pyrimidin-4-yl, pyrimidin-5-yl).

In some embodiments, R^{b1} is independently aryl. In some embodiments, R^{b1} is independently 6-10 member mono or bicyclic aryl. In some embodiments, R^{b1} is independently phenyl.

In some embodiments each R^{b1} is independently cycloalkylalkyl (e.g., cyclopropylmethyl, cyclobutylmethyl, cyclopentylmethyl, cyclohexylmethyl, cycloheptylmethyl). In some embodiments, each R^{b1} is independently heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, each R^{b1} is independently arylalkyl. In some embodiments, each R^{b1} is independently benzyl.

In some embodiments, each R^{b1} is independently heteroarylalkyl (e.g., pyridinylmethyl, thiazolylmethyl, triazolylmethyl, pyrazolylmethyl).

In one embodiment, provided is a compound of Formula (II) or a pharmaceutically acceptable salt, hydrate, solvate, prodrug, stereoisomer, or tautomer thereof; wherein:

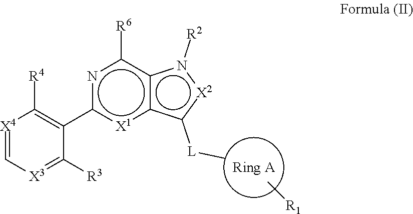

X¹ is selected from CH and N;
X² is selected from CR^{Xc2} and NR^{Xn2};
X³ is selected from CH and N;
X⁴ is selected from CH and N;
L is selected from —O—, —NRⁿ—, —S—, —S(═O)—, —S(═O)₂— and —CR^{c}R^{c′}—;
Ring A is selected from C₆-C₁₀ aryl, 5-10 membered heteroaryl and 3-10 membered heterocyclyl, each substituted with 0, 1 or 2 instances of halo or -Me;
R¹ is an optionally substituted 5-10 membered heteroaryl;
R² is absent or is selected from H, —C₁-C₆ alkyl, —C₃-C₉ cycloalkyl (e.g., cyclopropyl), —C₁-C₆ heteroalkyl, —C₁-C₆ haloalkyl, —C₁-C₆ hydroxyalkyl, arylalkyl, —S(═O)₂C₁-C₆alkyl (e.g., —S(═O)₂CH₃) and —C(═O)C₁-C₆alkyl (e.g., —C(═O)CH₃), wherein each hydrogen of the alkyl, haloalkyl, heteroalkyl, hydroxylalkyl and arylalkyl can be independently replaced with a deuterium atom;
R³ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR³, —N(R^{a3})₂, —C(═O)R^{a3}, —C(═O)OR^{a3}, —NR^{a3}C(═O)RV, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position.
R⁴ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NRS(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position;
each R^{Xc2} is independently selected from H, —C₁-C₆ alkyl, —C₁-C₆ heteroalkyl, —NH₂, —NH(C₁-C₆ alkyl) and —N(C₁-C₆ alkyl)₂;
each R^{Xn2} is absent or independently selected from H, —C₁-C₆ alkyl, —C₁-C₆ haloalkyl (e.g., CH₂CF₃), —S(═O)₂C₁-C₆alkyl (e.g., —S(═O)₂CH₁) and —C(═O)C₁-C₆alkyl (e.g., —C(═O)CH₃);
each Rⁿ is independently selected from H and —C₁-C₆ alkyl;
each R^{c} and R^{c′} is independently selected from H, —C₁-C₆ alkyl (e.g., -Me), —C₁-C₆ heteroalkyl, —C₁-C₆ haloalkyl, —OH, and —O(C₁-C₆ alkyl) (e.g., —OMe), or R^{c} and R^{c′} can be taken together with the atom to which they are attached to form a —C₁-C₉ cycloalkyl (e.g., cyclopropyl) or a carbonyl; and
each R^{a3} and R^{a4} is independently selected from H, —C₁-C₆ alkyl, —C₁-C₆ heteroalkyl, —C₁-C₆ haloalkyl, C₁-C₉ cycloalkyl, 3-7 membered heterocyclyl, cycloalkylalkyl, heterocyclylalkyl, aryl, 5-6 membered heteroaryl, arylalkyl and heteroarylalkyl wherein each hydrogen of the —C₁-C₆ alkyl can be independently replaced with a deuterium atom.

In certain embodiments, X¹ is CH. In other embodiments, X¹ is N.

In certain embodiments, X² is CR^{Xc2}. In other embodiments, X² is NR^{Xc2}. In some embodiments X¹ is N and X² is CR^{Xc2}. In some embodiments, X¹ is CH and X² is CR^{Xc2}. In some embodiments X¹ is N and X² is NR^{Xn2}. In some embodiments X¹ is CH and X² is NR^{Xn2}.

In some embodiments, the compound is of Formula (IIa), wherein:

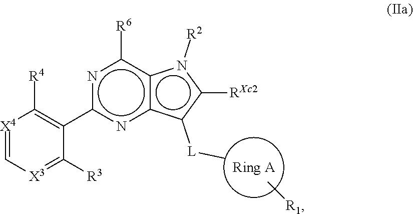

and wherein X³, X⁴, R³, R⁴, R⁶, R², R^{Xc2}, L, Ring A and R¹ are as defined herein.

In further embodiments, the compound is of Formula (IIa1), wherein:

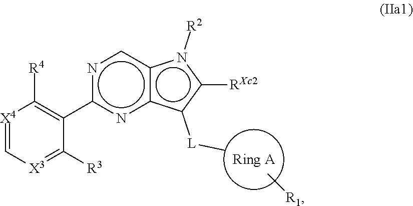

and wherein X³, X⁴, R³, R⁴, R², R^{Xc2}, L, Ring A and R¹ are as defined herein.

In certain embodiments, the compound is of Formula (IIb), wherein:

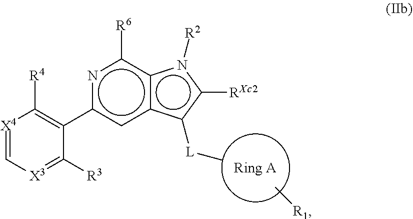

and wherein X³, X⁴, R³, R⁴, R⁶, R², R^{Xc2}, L, Ring A and R¹ are as defined herein.

In further embodiments the compound is of Formula (IIIb1), wherein:

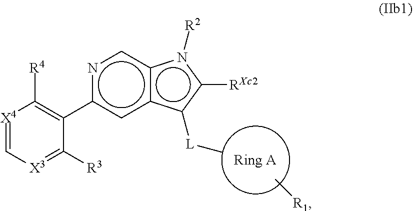

and wherein X³, X⁴, R³, R⁴, R², R^{Xc2}, L, Ring A and R¹ are as defined herein.

As generally defined herein, each R^{Xc2} is independently selected from H, -D, halo, —C₁-C₆ alkyl, —C₁-C₆ heteroalkyl, —NH₂, —NH(C₁-C₆ alkyl), —O(C₁-C₆ alkyl) and —N(C₁-C₆ alkyl)₂.

In some embodiments, each R² is independently selected from H, —F, -Me, —CH₂NMe₂, —CH₂NHMe, —CH₂OMe and —CH₂CH₂OMe.

In certain embodiments, each R^{Xc2} is independently selected from H and -Me.

In some embodiments, each R^{Xc2} is H.

In certain embodiments, R^{Xc2} is halo (e.g., fluoro, chloro, bromo, iodo). In further embodiments, R^{Xc2} is —Cl. In some embodiments, R^{Xc2} is —F. In some embodiments, R^{Xc2} is —Br. In some embodiments, R^{Xc2} is —I.

In certain embodiments, R^{Xc2} is —C₁-C₆ alkyl (e.g., -Me, -Et, —Pr, -ⁱPr, -ⁿBu, -sec-Bu, -iso-Bu, -^{t}Bu). In further embodiments, R^{Xc2} is -Me. In some embodiments, R^{Xc2} is -Et. In some embodiments R^{Xc2} is —Pr. In some embodiments, R^{Xc2} is -iPr.

In some embodiments, R^{Xc2} is —C₁-C₆ heteroalkyl. In further embodiments, R^{Xc2} is methoxymethyl (—CH₂OMe). In further embodiments, R¹² is methoxyethyl (—CH₂CH₂OMe). In some embodiments, R^{Xc2} is hydroxymethyl (—CH₂OH). In some embodiments, R^{Xc2} is aminomethyl (e.g., —CH₂NH₂, —CH₂NHCH₃, —CH₂N(CH₃)₂. In some embodiments, R^{Xc2} is —CH₂N(CH₃)CH₂CH₃.

In certain embodiments, the compound is of formula (IIc), wherein:

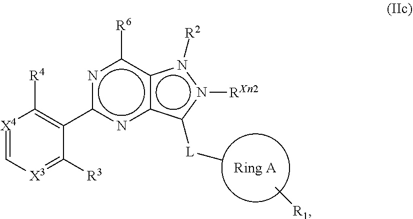

and wherein X³, X⁴, R³, R⁴, R⁶, R², R^{Xn2}, L, Ring A and R¹ are as defined herein.

In further embodiments, the compound is of formula (IIc1), wherein:

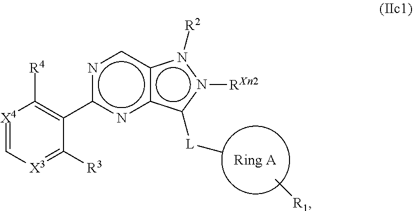

and wherein X³, X⁴, R³, R⁴, R², R^{Xn2}, L, Ring A and R¹ are as defined herein.

As generally defined herein, each R^{Xn2} is absent or independently selected from H, —C₁-C₆ alkyl —C₁-C₆ haloalkyl (e.g., CH₂CF₃), —S(═O)₂C₁-C₆alkyl (e.g., —S(═O)₂CH₃) and —C(═O)C₁-C₆ alkyl (e.g., —C(═O)CH₃).

In some embodiments, R^{Xn2} is absent or is selected from —H, -Me, —CH₂CF₃, —S(═O)₂Me. —C(═O)Me.

In some embodiments, R^{Xn2} is absent or is selected from —H and -Me. In some embodiments, Rx is absent.

In certain embodiments, the compound is of formula (IIc′), wherein:

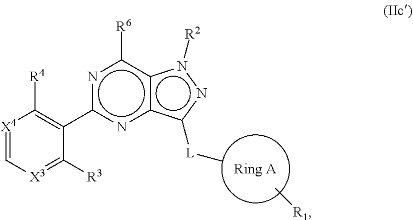

and wherein X³, X⁴, R³, R⁴, R⁶, R², L, Ring A and R¹ are as defined herein.

In further embodiments the compound is of formula (IIc′), wherein:

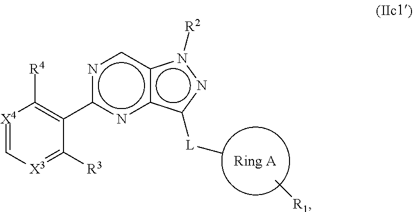

and wherein X³, X⁴, R³, R⁴, R², L, Ring A and R¹ are as defined herein.

In some embodiments, R^{Xn2} is —C₁-C₆ alkyl (e.g., -Me, -Et, —Pr, -ⁱPr, -ⁿBu, -sec-Bu, -iso-Bu, -^{t}Bu). In further embodiments, R^{Xn2} is -Me. In some embodiments, R^{Xn2} is -Et. In some embodiments R^{Xn2} is —Pr. In some embodiments, R^{Xn2} is -iPr.-Me.

In some embodiments, R^{Xn2} is —C₁-C₆ haloalkyl. In further embodiments, R^{Xn2} is trifluoromethyl (—CF₃). In other embodiments, R^{Xn2} is difluoromethyl (—CHF₂). In some embodiments, R^{Xn2} is trifluoroethyl (—CH₂CF₃).

In some embodiments, Rx is —C(═O)C₁-C₆ alkyl. In some embodiments, R^{Xn2} is —C(O)CH₃, —C(O)Bu, —C(O)ⁱPr, —C(O)Pr, or C(O)Bu. In some embodiments, R^{Xn2} is acetyl (—C(═O)Me).

In certain embodiments, R^{Xn2} is —S(═O)₂C₁-C₆ alkyl (e.g., —S(═O)₂Me, —S(═O)₂Et, —S(═O)₂Pr, —S(═O)₂ⁱPr). In some embodiments, R^{Xn2} is —S(═O)₂Me.

In certain embodiments, R^{Xn2} is —H.

In certain embodiments, X³ is CH. In other embodiments, X³ is N.

In some embodiments, X⁴ is CH. In alternate embodiments, X⁴ is N.

In some embodiments, both X³ and X⁴ are N. In other embodiments, X³ is CH and X¹ is N, or X³ is N and X⁴ is CH.

In some embodiments, the moiety represented by

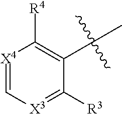

is selected from:

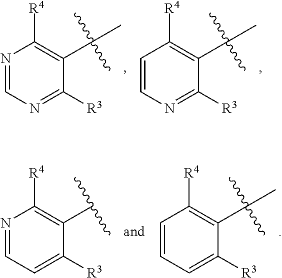

As generally defined herein, each R³ is independently selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)₂N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position, and wherein each R^{a3} is as described herein.

In some embodiments, each R³ is independently selected from H, -D, halo, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{a3} and —N(R^{a3})₂, wherein each alkyl, cycloalkyl and heterocyclyl is substituted with 0, 1, 2 or 3 instances of halo (e.g., —F, Cl), —OH, CN, -Me, -Et, —NH₂ or oxo and wherein each R^{a3} is independently selected from H, —C₁-C₆ alkyl, —C₁-C₆ heteroalkyl, —C₁-C₆ haloalkyl and C₃-C₉ cycloalkyl wherein each hydrogen atom of the C₁-C₆ alkyl of R can be independently replaced by deuterium;

In some embodiments, each R³ is independently selected from H, -D, halo, —C₁-C₆ alkyl (e.g., -Me, -Et, —Pr, -ⁱPr, -ⁿBu, -sec-Bu, -iso-Bu, -^{t}Bu), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃)—C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrolidinyl, piperidinyl, piperazinyl, morpholinyl, 6-oxa-1-azaspiro[3.3]heptanyl, 6-oxa-1-azaspiro[3.4]octanyl), —OR^{a3} and —N(R^{a3})₂, wherein each alkyl, cycloalkyl and heterocyclyl is substituted with 0, 1, 2 or 3 instances of halo (e.g., —F, Cl), and wherein each R^{a3} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl), wherein each hydrogen atom of the C₁-C₆ alkyl of R can be independently replaced by deuterium.

In some embodiments, each R³ is independently selected from H, -D, —Cl, -ⁱPr, —CH₂N(CH₃)CH₂CH₃, —CH₂N(CH₄)₂, —CF₃, —CH₂OH, cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OCH₂F, —OⁱPr, —OPr, —OMe, —OCD₃, OEt, —OH, —Ocyclopropyl, —N(Me)₂, —NHMe and —NHⁱPr.

In some embodiments, R³ is selected from cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OⁱPr, —OPr, —OMe, —OH, —Ocyclopropyl, —N(Me)₂, —NHMe and —NHⁱPr.

In some embodiments, R³ is selected from -ⁱPr, —CH₂N(CH₃)CH₂CH₃, —CH₂N(CH₃)₂, —CF₃, —CH₂OH and cyclopropyl.

In some embodiments, R³ is H. In some embodiments R³ is -D.

In certain embodiments, R³ is halo (e.g., fluoro, chloro, bromo, iodo). In further embodiments, R³ is —Cl. In some embodiments, R³ is —F. In some embodiments, R³ is —Br. In some embodiments, R³ is —I.

In some embodiments, R³ is —CN.

In certain embodiments, R³ is —C₁-C₆ alkyl (e.g., -Me, -Et, —Pr, —Pr, -ⁿBu, -sec-Bu, -iso-Bu, -^{t}Bu). In further embodiments, R³ is -Me. In some embodiments, R³ is -Et. In some embodiments R³ is —Pr. In some embodiments, R³ is -iPr.

In some embodiments, R³ is —C₁-C₆ heteroalkyl. In further embodiments, R³ is methoxymethyl (—CH₂OCH₃). In some embodiments, R³ is hydroxymethyl (—CH₂OH). In some embodiments, R³ is aminomethyl (e.g., —CH₂NH₂, —CH₂NHCH₃, —CH₂N(CH₃)₂. In some embodiments, R³ is —CH₂N(CH₃)CH₂CH₃.

In some embodiments, R³ is —C₁-C₆ haloalkyl. In further embodiments, R³ is trifluoromethyl (—CF₃). In other embodiments, R³ is difluoromethyl (—CHF₂). In some embodiments, R³ is fluoromethyl (—CH₂F).

In some embodiments, R³ is —C₁-C₆ hydroxyalkyl (e.g., —CH₂OH, —CH₂CH₂OH).

In some embodiments, R³ is —C₃-C₁₀ cycloalkyl (e.g., cyclopropyl, cyclobutyl, cyclopentyl, cyclohexyl). In some embodiments, R³ is cyclopropyl. In some embodiments R³ is cyclobutyl. In some embodiments, R³ is cyclopentyl. In some embodiments, R³ is cyclohexyl.

In some embodiments, R³ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3.3]heptanyl, 6-oxa-1-azaspiro[3.4]octanyl). In some embodiments, R³ is oxetanyl. In some embodiments, R³ is tetrahydropyranyl. In some embodiments, R³ is tetrahydrofuranyl. In some embodiments, R³ is azetidinyl. In some embodiments, R³ is pyrrolidinyl. In some embodiments, R³ is piperidinyl. In some embodiments, R³ is piperazinyl. In some embodiments, R³ is morpholinyl. In some embodiments, R³ is azepanyl. In some embodiments, R³ is 6-oxa-1-azaspiro[3.3]heptanyl. In some embodiments, R⁶ is 6-oxa-1-azaspiro[3.4]octanyl.

In some embodiments R³ is cycloalkylalkyl (e.g., cyclopropylmethyl, cyclobutylmethyl, cyclopentylmethyl, cyclohexylmethyl, cycloheptylmethyl). In some embodiments, R³ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R³ is arylalkyl. In some embodiments, R³ is benzyl.

In some embodiments, R³ is heteroarylalkyl (e.g., pyridinylmethyl, thiazolylmethyl, triazolylmethyl, pyrazolylmethyl).

In some embodiments, R³ is —OR^{a3} (e.g., hydroxy (—OH), methoxy, difluoromethoxy (—OCHF₂), trifluoromethoxy (—OCF₃), —OCH(CH₃)CF₃, —OCH₂CF₃, ethoxy, propoxy, isopropoxy, cyclopropyloxy, cyclobutyloxy). In some embodiments, one or more of the hydrogen atoms of R^{a3} are replaced with deuterium. In some embodiments, R³ is hydroxy. In some embodiments, R³ is methoxy. In some embodiments, R³ is —OCD₃. In some embodiments, R³ is ethoxy. In some embodiments, R³ is propoxy. In some embodiments, R³ is isopropoxy. In some embodiments R³ is difluoromethoxy, (—OCHF₂). In some embodiments R₃ is fluoromethoxy, (—OCH₂F). In some embodiments, R³ is trifluoromethoxy (—OCF₃). In some embodiments, R³ is —OCH(CH₃)CF₃. In some embodiments, R³ is —OCH₂CF₃. In some embodiments, R³ is cyclopropyloxy.

In some embodiments, R³ is —N(R^{a3})₂ (e.g., —NH₂, —NHR^{a3}, —N(CH₃)R^{a3}). In some embodiments, R³ is —NH₂. In some embodiments, R³ is —NHR^{a3} (e.g., —NHMe, —NHEt, —NHPr, —NHⁱPr, —NHcyclopropyl, —NHcyclobutyl). In some embodiments, R³ is —N(CH₃)R^{a3} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R³ is —C(═O)R^{a3} or —C(═O)OR^{a3}. In some embodiments, R³ is —C(═O)R^{a3} wherein R^{a3} is as described herein. In some embodiments, R³ is —C(═O)alkyl. In some embodiments, R³ is —C(O)CH₃, —C(O)cyclopropyl, —C(O)cyclobutyl, —C(O)Bu, —C(O)ⁱPr, —C(O)Pr, —C(O)^{t}Bu, or —C(═O)OMe. In some embodiments, R³ is acetyl (—C(═O)Me). In some embodiments, R³ is —C(═O)OR^{a3}. In some embodiments, R³ is —COOH. In some embodiments, R³ is COOMe.

In some embodiments, R³ is —NR^{a3}C(═O)R³. In certain embodiments, R³ is —NHC(═O)R^{a3} (e.g., NHC(═O)Me, NHC(═O)Et, NHC(═O)Pr, NHC(═O)ⁱPr, NHC(═O)Bu, NHC(═O)^{t}Bu, NHC(═O)Cyclopropyl, NHC(═O)Cyclobutyl). In some embodiments, R³ is —N(CH₃)C(═O)R^{a3} (e.g., N(CH₃)C(═O)Me, N(CH₃)C(═O)Et, N(CH₃)C(═O)Pr, N(CH₃)C(═O)ⁱPr, N(CH₃)C(═O)Bu, N(CH₃)C(═O)^{t}Bu, N(CH₃)C(═O)Cyclopropyl, N(CH₃)C(═O)Cyclobutyl).

In some embodiments, R³ is —NR^{a3}C(═O)OR^{a3}. In certain embodiments, R³ is —NHC(═O)OR^{a3} (e.g., NHC(═O)OMe, NHC(═O)OEt, NHC(═O)OPr, NHC(═O)OⁱPr, NHC(═O)OBu, NHC(═O)O^{t}Bu, NHC(═O)Ocyclopropyl, NHC(═O)Ocyclobutyl). In some embodiments, R³ is —N(CH₃)C(═O)OR^{a3} (e.g., N(CH₃)C(═O)OMe, N(CH₃)C(═O)OEt, N(CH₃)C(═O)OPr, N(CH₃)C(═O)OⁱPr, N(CH₃)C(═O)OBu, N(CH₃)C(═O)O^{t}Bu, N(CH₃)C(═O)OCyclopropyl, N(CH₃)C(═O)OCyclobutyl).

In some embodiments, R³ is —C(═O)N(R^{a3})₂(e.g., —C(═O)NH₂, —C(═O)NHR^{a3}, —C(═O)N(CH₃)R^{a3}). In some embodiments, R³ is —C(═O)NH₂. In certain embodiments, R³ is —C(═O)NH^{a3} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R³ is —C(═O)N(CH₃)R^{a3} (e.g., —C(═O)NMe₂, —C(═O)N(CH₃)Et, —C(═O)N(CH₃)Pr, —C(═O)N(CH₃)ⁱPr, —C(═O)N(CH)Bu, —C(═O)N(CH₃)^{t}Bu, —C(═O)N(CH₃)Cyclopropyl, —C(═O)N(CH₃)Cyclobutyl).

In some embodiments, R³ is —OC(═O)N(R^{a3})₂. In certain embodiments, R³ is —OC(═O)NHR^{a3} (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R³ is —OC(═O)N(CH₃)R^{a3} (e.g., —OC(═O)NMe₂, —OC(═O)N(CH₃)Et, —OC(═O)N(CH₃)Pr, —OC(═O)N(CH)Pr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R³ is —S(═O)R^{a3}. In certain embodiments, R³ is —S(═O)alkyl (e.g., —S(═O)Me, —S(═O)Et, —S(═O)Pr, —S(═O)ⁱPr). In certain embodiments, R³ is —S(═O)cycloalkyl (e.g., —S(═O)cyclopropyl, —S(═O)cyclobutyl, —S(═O)cyclopentyl, —S(═O)cyclohexyl).

In some embodiments, R³ is —S(═O)₂R^{a3}. In certain embodiments, R³ is —S(═O)₂alkyl (e.g., —S(═O)₂Me, —S(═O)₂Et, —S(═O)₂Pr, —S(═O)₂ⁱPr). In certain embodiments, R³ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R³ is S(═O)₂aryl (e.g., S(═O)₂phenyl).

In some embodiments, R³ is —SR^{a3}. In certain embodiments, R³ is —Salkyl (e.g., —SMe, —SEt, —SPr, —SⁱPr). In certain embodiments, R³ is —Scycloalkyl (e.g., —Scyclopropyl, —Scyclobutyl, —Scyclopentyl, —Scyclohexyl). In certain embodiments, R³ is —Saryl (e.g., Sphenyl).

In some embodiments, R³ is —S(═O)(═NR^{a3})R^{a3}. In certain embodiments, R³ is —S(═O)(═NH)R^{a3} (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R³ is —S(═O)(═NCH₃)R^{a3} (e.g., —S(═O)(═NCH₃)Me, —S(═O)(═NCH)Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R³ is —NR^{a3}S(═O)₂R³. In certain embodiments, R³ is —NHS(═O)₂alkyl (e.g., —NHS(═O)₂Me, —NHS(═O)₂Et, —NHS(═O)₂Pr, —NHS(═O)₂ⁱPr). In certain embodiments, R³ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R³ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂ⁱPr). In certain embodiments, R³ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R³ is —S(═O)₂N(R^{a3})₂. (e.g., —S(═O)₂NH₂, —S(═O)₂NHR^{a3}, —S(═O)₂N(CH₃)R^{a3}). In some embodiments, R³ is —S(═O)₂NH₂. In some embodiments, R³ is —S(═O)₂NHR^{a3} (e g., —S(═O)₂NHMe, —S(═O)₂NHEt, —S(═O)₂NHPr, —S(═O)₂NHⁱPr, —S(═O)₂NHcyclopropyl, —S(═O)₂NHcyclobutyl). In some embodiments, R³ is —S(═O)₂N(CH₃)R^{a3} (e.g., —S(═O)₂NMe₂, —S(═O)₂N(CH₃)Et, —S(═O)₂N(CH₃)Pr, —S(═O)₂N(CH₃)ⁱPr, —S(═O)₂N(CH)cyclopropyl, —S(═O)₂N(CH)cyclobutyl).

As generally defined herein, each R^{a3} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl, wherein each hydrogen atom of the C₁-C₆ alkyl of R^{a3} can be independently replaced by deuterium.

In some embodiments, each R^{a3} is independently selected from H, —C₁-C₆ alkyl (e.g., -Me, -Et, —Pr, -ⁱPr, -ⁿBu, -^{t}Bu, -sec-Bu, -iso-Bu) and —C₁-C₆ haloalkyl (e.g., —CHF₂, —CF₃, —CH(CH₃)CF₃, —CH₂CF₃), wherein each hydrogen atom of the C₁-C₆ alkyl of R^{a3} can be independently replaced by deuterium (e.g., -CD₃).

In some embodiments, each R^{a3} is independently H.

In some embodiments, each R^{a3} is independently —C₁-C₆ alkyl (e.g., -Me, -Et, —Pr, -ⁱPr, -ⁿBu, -^{t}Bu, -sec-Bu, -iso-Bu). In some embodiments, each R^{a3} is independently -Me. In some embodiments, each R is independently -Et. In some embodiments, each R is independently —Pr. In some embodiments, each R^{a3} is independently -ⁱPr. In some embodiments, one or more hydrogen atoms of the C₁-C₆ alkyl can be independently replaced by deuterium. In some embodiments, each R^{a3} is independently —CD₃.

In some embodiments, each R^{a3} is independently —C₁-C₆ heteroalkyl. In further embodiments, each R^{a3} is independently methoxymethyl (—CH₂OCH₃). In some embodiments, each R is independently hydroxymethyl (—CH₂OH). In some embodiments, each R^{a3} is independently is aminomethyl (e.g., —CH₂NH₂, —CH₂NHCH₃, —CH₂N(CH₃)₂.

In some embodiments, each R^{a3} is independently —C₁-C₆ haloalkyl. In further embodiments, each R^{a3} is independently trifluoromethyl (—CF₃). In other embodiments, each R^{a3} is independently difluoromethyl (—CHF₂). In some embodiments, each R^{a3} is —CH(CH₃)CF₃. In some embodiments, each R^{a3} is —CH₂CF₃.

In some embodiments, each R^{a3} is independently —C₃-C₉ cycloalkyl (e.g., cyclopropyl, cyclobutyl, cyclopentyl, cyclohexyl). In some embodiments, each R^{a3} is independently cyclopropyl. In some embodiments each R^{a3} is independently cyclobutyl. In some embodiments, each R^{a3} is independently cyclopentyl. In some embodiments, each R^{a3} is independently cyclohexyl.

In some embodiments, each R^{a3} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{a3} is independently heteroaryl. In some embodiments, R^{a3} is independently a 5-10 member heteroaryl (e.g., a 5-6 member monocyclic heteroaryl or an 8-10 member bicyclic heteroaryl containing 1-3 heteroatoms independently selected from N, O and S). In some embodiments, R is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{a3} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{a3} is independently thiophenyl (e.g., thiophen-2-yl, thiophen-3-yl). In some embodiments, R^{a3} is independently pyrazolyl (e.g., pyrazol-1-yl, pyrazol-3-yl, pyrazol-5-yl). In some embodiments, R^{a3} is independently thiazolyl (e.g., thiazol-2-yl, thiazol-4-yl, thiazol-5-yl). In some embodiments, R^{a3} is independently a 6-member monocyclic heteroaryl (e.g., pyridyl, pyrimidinyl, triazinyl, pyrazinyl, pyridazinyl). In some embodiments, R^{a3} is independently pyridinyl (e.g., pyridin-2-yl, pyridin-3-yl, pyridin-4-yl). In some embodiments, R^{a3} is independently pyrimidinyl (e.g., pyrimidin-2-yl, pyrimidin-4-yl, pyrimidin-5-yl).

In some embodiments, R^{a3} is independently aryl. In some embodiments, R^{a3} is independently 6-10 member mono or bicyclic aryl. In some embodiments, R^{a3} is independently phenyl.

In some embodiments each R^{a3} is independently cycloalkylalkyl (e.g., cyclopropylmethyl, cyclobutylmethyl, cyclopentylmethyl, cyclohexylmethyl, cycloheptylmethyl). In some embodiments, each R^{a3} is independently heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, each R^{a3} is independently arylalkyl. In some embodiments, each R is independently benzyl.

In some embodiments, each R^{a3} is independently heteroarylalkyl (e.g., pyridinylmethyl, thiazolylmethyl, triazolylmethyl, pyrazolylmethyl).

As generally defined herein, each R⁴ is independently selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position

In some embodiments, each R⁴ is independently selected from H, -D, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{a4} and —N(R^{a4})₂, wherein each alkyl, cycloalkyl and heterocyclyl is substituted with 0, 1, 2 or 3 instances of halo (e.g., —F, —Cl), —OH, CN, -Me, -Et, —NH₂ or oxo and wherein each R^{a4} is independently selected from H, —C₁-C₆ alkyl, —C₁-C₆ heteroalkyl, —C₁-C₆ haloalkyl and C₃-C₉ cycloalkyl;

In some embodiments, each R⁴ is independently selected from H, -D, —C₁-C₆ alkyl (e.g., -Me, -Et, —Pr, -ⁱPr, -ⁿBu, -sec-Bu, -iso-Bu, -^{t}Bu), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃)—C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrolidinyl, piperidinyl, piperazinyl, morpholinyl, 6-oxa-1-azaspiro[3.3]heptanyl, 6-oxa-1-azaspiro[3.4]octanyl), —OR^{a4} and —N(R^{a4})₂, wherein each alkyl, cycloalkyl and heterocyclyl is substituted with 0, 1, 2 or 3 instances of halo (e.g., —F, —Cl), and wherein each R^{a4} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R⁴ is independently selected from H, -D, -ⁱPr, —CH₂N(CH₃)CH₂CH₃, —CH₂N(CH₃)₂, —CF₃, —CH₂OH, cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OⁱPr, —OPr, —OMe, —OH, —Ocyclopropyl, —N(Me)₂, —NHMe and —NHⁱPr.

In some embodiments, each R⁴ is selected from H and cyclopropyl.

In some embodiments, R⁴ is H. In some embodiments R⁴ is -D.

In certain embodiments, R⁴ is halo (e.g., fluoro, chloro, bromo, iodo). In further embodiments, R⁴ is —Cl. In some embodiments, R⁴ is —F. In some embodiments, R⁴ is —Br. In some embodiments, R⁴ is —I.

In some embodiments, R⁴ is —CN.

In certain embodiments, R⁴ is —C₁-C₆ alkyl (e.g., -Me, -Et, —Pr, -ⁱPr, -ⁿBu, -sec-Bu, -iso-Bu, -^{t}Bu). In further embodiments, R⁴ is -Me. In some embodiments, R⁴ is -Et. In some embodiments R⁴ is —Pr. In some embodiments, R⁴ is -iPr. In some embodiments, R⁴ is -^{t}Bu.

In some embodiments, R⁴ is —C₁-C₆ heteroalkyl. In further embodiments, R⁴ is methoxymethyl (—CH₂OCH₃). In some embodiments, R⁴ is hydroxymethyl (—CH₂OH). In some embodiments, R⁴ is aminomethyl (e.g., —CH₂NH₂, —CH₂NHCH₃, —CH₂N(CH₃)₂. In some embodiments, R⁴ is —CH₂N(CH₃)CH₂CH₃.

In some embodiments, R⁴ is —C₁-C₆ haloalkyl. In further embodiments, R⁴ is trifluoromethyl (—CF₃). In other embodiments, R⁴ is difluoromethyl (—CHF₂).

In some embodiments, R⁴ is —C₁-C₆ hydroxyalkyl (e.g., —CH₂OH, —CH₂CH₂OH, —CH(OH)(CH₃)₂). In some embodiments, R⁴ is —CH(OH)(CH₃)₂.

In some embodiments, R⁴ is —C₃-C₁₀ cycloalkyl (e.g., cyclopropyl, cyclobutyl, cyclopentyl, cyclohexyl). In some embodiments, the cycloalkyl is substituted with 0, 1, 2 or 3 instances of halo (e.g., —F, —Cl), —OH, CN, -Me, -Et, —NH₂ or oxo In some embodiments, R⁴ is cyclopropyl. In some embodiments, the cyclopropyl is unsubstituted. In some embodiments, the cyclopropyl is substituted with 1 or 2 instances of —F. In some embodiments, R⁴ is 1-F-cyclopropyl. In some embodiments, R⁴ is 2,2-difluorocyclopropyl. In some embodiments R is cyclobutyl. In some embodiments, R⁴ is cyclopentyl. In some embodiments, R⁴ is cyclohexyl.

In some embodiments, R⁴ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3,3]heptanyl, 6-oxa-1-azaspiro[3.4]octanyl). In some embodiments, R⁴ is oxetanyl. In some embodiments, R⁴ is tetrahydropyranyl. In some embodiments, R⁴ is tetrahydrofuranyl. In some embodiments, R⁴ is azetidinyl. In some embodiments, R⁴ is pyrrolidinyl. In some embodiments, R⁴ is piperidinyl. In some embodiments, R⁴ is piperazinyl. In some embodiments, R⁴ is morpholinyl. In some embodiments, R⁴ is azepanyl. In some embodiments, R⁴ is 6-oxa-1-azaspiro[3.3]heptanyl. In some embodiments, R⁶ is 6-oxa-1-azaspiro[3.4]octanyl.

In some embodiments R⁴ is cycloalkylalkyl (e.g., cyclopropylmethyl, cyclobutylmethyl, cyclopentylmethyl, cyclohexylmethyl, cycloheptylmethyl). In some embodiments, R⁴ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R⁴ is arylalkyl. In some embodiments, R⁴ is benzyl.

In some embodiments, R⁴ is heteroarylalkyl (e.g., pyridinylmethyl, thiazolylmethyl, triazolylmethyl, pyrazolylmethyl).

In some embodiments, R⁴ is —OR^{a4} (e.g., hydroxy (—OH), methoxy, difluoromethoxy (—OCHF₂), trifluoromethoxy (—OCF₃), —OCH(CH₃)CF₃, —OCH₂CF₃, ethoxy, propoxy, isopropoxy, cyclopropyloxy, cyclobutyloxy). In some embodiments, R⁴ is hydroxy. In some embodiments, R⁴ is methoxy. In some embodiments, R⁴ is ethoxy. In some embodiments, R⁴ is propoxy. In some embodiments, R⁴ is isopropoxy. In some embodiments R⁴ is difluoromethoxy. (—OCHF₂). In some embodiments, R⁴ is trifluoromethoxy (—OCF₃). In some embodiments, R⁴ is —OCH(CH₃)CF₃. In some embodiments, R⁴ is —OCH₂CF₃. In some embodiments, R⁴ is cyclopropyloxy.

In some embodiments, R⁴ is —N(R^{a4})₂ (e.g., —NH₂, —NHR^{a4}, —N(CH₃)R^{a4}). In some embodiments, R⁴ is —NH₂. In some embodiments, R⁴ is —NHR^{a4} (e.g., —NHMe, —NHEt, —NHPr, —NHⁱPr, —NHcyclopropyl, —NHcyclobutyl). In some embodiments, R⁴ is —N(CH₃)R^{a4} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R⁴ is —C(═O)R^{a4} or —C(═O)OR^{a4}. In some embodiments, R⁴ is —C(═O)R^{a4} wherein R^{a4} is as described herein. In some embodiments, R⁴ is —C(═O)alkyl. In some embodiments, R⁴ is —C(O)CH₃, —C(O)cyclopropyl, —C(O)cyclobutyl, —C(O)Bu, —C(O)ⁱPr, —C(O)Pr, —C(O)^{t}Bu, or —C(═O)OMe. In some embodiments, R⁴ is acetyl (—C(═O)Me). In some embodiments, R⁴ is —C(═O)OR^{a4}. In some embodiments, R⁴ is —COOH. In some embodiments, R⁴ is COOMe.

In some embodiments, R⁴ is —NR^{a4}C(═O)R^{a4}. In certain embodiments, R⁴ is —NHC(═O)R^{a4} (e.g., NHC(═O)Me, NHC(═O)Et, NHC(═O)Pr, NHC(═O)ⁱPr, NHC(═O)Bu, NHC(═O)^{t}Bu, NHC(═O)Cyclopropyl, NHC(═O)Cyclobutyl). In some embodiments, R⁴ is —N(CH₃)C(═O)R^{a4} (e.g., N(CH₃)C(═O)Me, N(CH₃)C(═O)Et, N(CH₃)C(═O)Pr, N(CH₃)C(═O)ⁱPr, N(CH₃)C(═O)Bu, N(CH₃)C(═O)^{t}Bu, N(CH₃)C(═O)Cyclopropyl, N(CH₃)C(═O)Cyclobutyl).

In some embodiments, R⁴ is —NR^{a4}C(═O)OR^{a4}. In certain embodiments, R⁴ is —NHC(═O)OR^{a4} (e.g., NHC(═O)OMe, NHC(═O)OEt, NHC(═O)OPr, NHC(═O)OⁱPr, NHC(═O)OBu, NHC(═O)O^{t}Bu, NHC(═O)OCyclopropyl, NHC(═O)OCyclobutyl). In some embodiments, R⁴ is —N(CH₃)C(═O)OR^{a4} (e.g., N(CH₃)C(═O)OMe, N(CH₃)C(═O)OEt, N(CH₃)C(═O)OPr, N(CH₃)C(═O)OⁱPr, N(CH₃)C(═O)OBu, N(CH₃)C(═O)O^{t}Bu, N(CH₃)C(═O)OCyclopropyl, N(CH₃)C(═O)OCyclobutyl).

In some embodiments, R⁴ is —C(═O)N(R⁴)₂(e.g., —C(═O)NH₂, —C(═O)NHR^{a4}, —C(═O)N(CH₃)R^{a4}). In some embodiments, R⁴ is —C(═O)NH₂. In certain embodiments, R is —C(═O)NHR^{a4} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁴ is —C(═O)N(CH₃)R^{a4} (e.g., —C(═O)NMe₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH)Cyclopropyl, —C(═O)N(CH₃)Cyclobutyl).

In some embodiments, R⁴ is —OC(═O)N(R^{a4})₂. In certain embodiments, R⁴ is —OC(═O)NHR^{a4} (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁴ is —OC(═O)N(CH₃)R^{a4} (e.g., —OC(═O)NMe₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁴ is —S(═O)R^{a4}. In certain embodiments, R⁴ is —S(═O)alkyl (e.g., —S(═O)Me, —S(═O)Et, —S(═O)Pr, —S(═O)ⁱPr). In certain embodiments, R⁴ is —S(═O)cycloalkyl (e.g., —S(═O)cyclopropyl, —S(═O)cyclobutyl, —S(═O)cyclopentyl, —S(═O)cyclohexyl).

In some embodiments, R⁴ is —S(═O)₂R^{a4}. In certain embodiments, R⁴ is —S(═O)₂alkyl (e.g., —S(═O)₂Me, —S(═O)₂Et, —S(═O)₂Pr, —S(═O)₂ⁱPr). In certain embodiments, R⁴ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁴ is S(═O)₂aryl (e.g., S(═O)₂phenyl).

In some embodiments, R⁴ is —SR^{a4}. In certain embodiments, R⁴ is —Salkyl (e.g., —SMe, —SEt, —SPr, —SⁱPr). In certain embodiments, R⁴ is —Scycloalkyl (e.g., —Scyclopropyl, —Scyclobutyl, —Scyclopentyl, —Scyclohexyl). In certain embodiments, R⁴ is —Saryl (e.g., Sphenyl).

In some embodiments, R⁴ is —S(═O)(═NR^{a4})R⁴. In certain embodiments, R⁴ is —S(═O)(═NH)R^{a4} (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁴ is —S(═O)(═NCH₃)R^{a4} (e.g., —S(═O)(═NCH₃)Me, —S(═O)(═NCH₃)Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁴ is —NR^{a4}S(═O)₂R^{a4}. In certain embodiments, R⁴ is —NHS(═O)₂alkyl (e.g., —NHS(═O)₂Me, —NHS(═O)₂Et, —NHS(═O)₂Pr, —NHS(═O)₂ⁱPr). In certain embodiments, R⁴ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁴ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂ⁱPr). In certain embodiments, R⁴ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁴ is —S(═O)₂N(R^{a4})₂. (e.g., —S(═O)₂NH₂, —S(═O)₂NHR^{a4}, —S(═O)₂N(CH₃)R^{a4}). In some embodiments, R⁴ is —S(═O)₂NH₂. In some embodiments, R⁴ is —S(═O)₂NHR^{a4} (e.g., —S(═O)₂NHMe, —S(═O)₂NHEt, —S(═O)₂NHPr, —S(═O)₂NHⁱPr, —S(═O)₂NHcyclopropyl, —S(═O)₂NHcyclobutyl). In some embodiments, R⁴ is —S(═O)₂N(CH₃)R^{a4} (e.g., —S(═O)₂NMe₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R^{a4} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R^{a4} is independently selected from H, —C₁-C₆ alkyl (e.g., -Me, -Et, —Pr, -ⁱPr, -ⁿBu, -^{t}Bu, -sec-Bu, -iso-Bu) and —C₁-C₆ haloalkyl (e.g., —CHF₂, —CF₃, —CH(CH₃)CF₃, —CH₂CF₃).

In some embodiments, each R^{a4} is independently H.

In some embodiments, each R^{a4} is independently —C₁-C₆ alkyl (e.g., -Me, -Et, —Pr, -ⁱPr, -ⁿBu, -^{t}Bu, -sec-Bu, -iso-Bu). In some embodiments, each R^{a4} is independently -Me. In some embodiments, each R^{a4} is independently -Et. In some embodiments, each R^{a4} is independently —Pr. In some embodiments, each R^{a4} is independently -ⁱPr.

In some embodiments, each R^{a4} is independently —C₁-C₆ heteroalkyl. In further embodiments, each R^{a4} is independently methoxymethyl (—CH₂OCH₃). In some embodiments, each R^{a4} is independently hydroxymethyl (—CH₂OH). In some embodiments, each R^{a4} is independently is aminomethyl (e.g., —CH₂NH₂, —CH₂NHCH₃, —CH₂N(CH₃)₂.

In some embodiments, each R^{a4} is independently —C₁-C₆ haloalkyl. In further embodiments, each R^{a4} is independently trifluoromethyl (—CF₃). In other embodiments, each R^{a4} is independently difluoromethyl (—CHF₂). In some embodiments, each R^{a4} is —CH(CH₃)CF₃. In some embodiments, each R^{a4} is —CH₂CF₃.

In some embodiments, each R^{a4} is independently —C₃-C₉ cycloalkyl (e.g., cyclopropyl, cyclobutyl, cyclopentyl, cyclohexyl). In some embodiments, each R^{a4} is independently cyclopropyl. In some embodiments each R^{a4} is independently cyclobutyl. In some embodiments, each R^{a4} is independently cyclopentyl. In some embodiments, each R^{a4} is independently cyclohexyl.

In some embodiments, each R^{a4} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{a4} is independently heteroaryl. In some embodiments, R^{a4} is independently a 5-10 member heteroaryl (e.g., a 5-6 member monocyclic heteroaryl or an 8-10 member bicyclic heteroaryl containing 1-3 heteroatoms independently selected from N, O and S). In some embodiments, R is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{a4} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{a4} is independently thiophenyl (e.g., thiophen-2-yl, thiophen-3-yl). In some embodiments, R^{a4} is independently pyrazolyl (e.g. pyrazol-1-yl, pyrazol-3-yl, pyrazol-5-yl). In some embodiments, R^{a4} is independently thiazolyl (e.g., thiazol-2-yl, thiazol-4-yl, thiazol-5-yl). In some embodiments, R^{a4} is independently a 6-member monocyclic heteroaryl (e.g., pyridyl, pyrimidinyl, triazinyl, pyrazinyl, pyridazinyl). In some embodiments, R^{a4} is independently pyridinyl (e.g., pyridin-2-yl, pyridin-3-yl, pyridin-4-yl). In some embodiments, R^{a4} is independently pyrimidinyl (e.g., pyrimidin-2-yl, pyrimidin-4-yl, pyrimidin-5-yl).

In some embodiments, R^{a4} is independently aryl. In some embodiments, R^{a4} is independently 6-10 member mono or bicyclic aryl. In some embodiments, R^{a4} is independently phenyl.

In some embodiments each R^{a4} is independently cycloalkylalkyl (e.g., cyclopropylmethyl, cyclobutylmethyl, cyclopentylmethyl, cyclohexylmethyl, cycloheptylmethyl). In some embodiments, each R^{a4} is independently heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, each R^{a4} is independently arylalkyl. In some embodiments, each R^{a4} is independently benzyl.

In some embodiments, each R^{a4} is independently heteroarylalkyl (e.g., pyridinylmethyl, thiazolylmethyl, triazolylmethyl, pyrazolylmethyl).

In certain embodiments, the moiety represented by

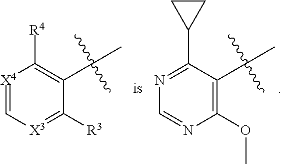

In some embodiments, the moiety represented by

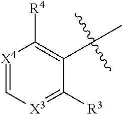

is selected from

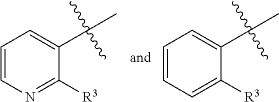

wherein R³ is as defined herein.

In some embodiments, the moiety represented by

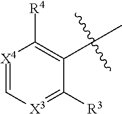

is

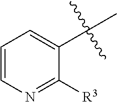

wherein R³ is as defined herein. In further embodiments, R³ is selected from cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OⁱPr, —OPr, —OMe, —OH, —Ocyclopropyl, —N(Me)₂, —NHMe and —NHⁱPr.

In some embodiments, the moiety represented by

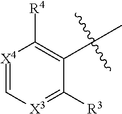

is

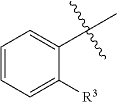

wherein R³ is as defined herein. In further embodiments, R³ is selected from -ⁱPr, —CH₂N(CH₃)CH₂CH₃, —CH₂N(CH₃)₂, —CF₃, —CH₂OH and cyclopropyl.

As generally defined herein, L is selected from —O—, —NRⁿ—, —S—, —S(═O)—, —S(═O)₂— and —CR^{c}R^{c′}—.

In certain embodiments, L is —O—.

In certain embodiments, L is —NRⁿ—.

As generally defined herein, each Rⁿ is independently selected from H and —C₁-C₆ alkyl. In some embodiments, Rⁿ is selected from H and Me. In certain embodiments, Rⁿ is —C₁-C₆ alkyl (e.g., -Me, -Et, —Pr, -ⁱPr, -ⁿBu, -sec-Bu, -iso-Bu, -^{t}Bu). In further embodiments, Rⁿ is -Me. In some embodiments, Rⁿ is -Et. In some embodiments Rⁿ is —Pr. In some embodiments, Rⁿ is -iPr. In some embodiments, Rⁿ is H.

In certain embodiments, L is —S—.

In certain embodiments, L is —S(═O)—.

In certain embodiments, L is —S(═O)₂—.

In certain embodiments, L is —CR^{c}R^{c′}—.

As generally defined herein, each R^{c} and R^{c′} is independently selected from H, —C₁-C₆ alkyl —C₁-C₆ heteroalkyl, —C₁-C₆ haloalkyl, —OH, and —O(C₁-C₆ alkyl) or R^{c} and R^{c′} can be taken together with the atom to which they are attached to form a —C₃-C₉ cycloalkyl or a carbonyl.

In some embodiments, R^{c} and R^{c′} are each independently selected from H, -Me, —OH, —OMe or are taken together to form a carbonyl group or a cyclopropyl group.

In some embodiments, R^{c} is H and R^{c′} is selected from -Me, —OH and —OMe

In certain embodiments, R^{c} and R^{c′} are each independently H.

In certain embodiments, R^{c} and R^{c′} are each independently —C₁-C₆ alkyl (e.g., -Me, -Et, —Pr, -ⁱPr, -ⁿBu, -sec-Bu, -iso-Bu, -^{t}Bu). In further embodiments, R^{c} and R^{c′} are each independently -Me. In some embodiments, R^{c} and R^{c′} are each independently -Et. In some embodiments R^{c} and R^{c′} are each independently —Pr. In some embodiments, R^{c} and R^{c′} are each independently -iPr.

In some embodiments, R^{c} and R^{c′} are each independently —C₁-C₆ heteroalkyl. In further embodiments, R^{c} and R^{c′} are each independently methoxymethyl (—CH₂OCH₃). In some embodiments, R^{c} and R^{c′} are each independently hydroxymethyl (—CH₂OH). In some embodiments, R^{c} and R^{c′} are each independently aminomethyl (e.g., —CH₂NH₂, —CH₂NHCH₃, —CH₂N(CH₃)₂. In some embodiments, R^{c} and R^{c′} are each independently —CH₂N(CH₃)CH₂CH₃.

In some embodiments, R^{c} and R^{c′} are each independently —C₁-C₆ haloalkyl. In further embodiments, R^{c} and R^{c′} are each independently trifluoromethyl (—CF₃). In other embodiments, R^{c} and R^{c′} are each independently difluoromethyl (—CHF₂).

In some embodiments, R^{c} and R^{c′} are each independently hydroxy (—OH). In some embodiments, R^{c} and R^{c′} are each independently —O(C₁-C₆ alkyl) (e.g., methoxy, ethoxy, propoxy, isopropoxy). In some embodiments, R^{c} and R^{c′} are each independently methoxy. In some embodiments, R^{c} and R^{c′} are each independently ethoxy. In some embodiments, R^{c} and R^{c′} are each independently propoxy. In some embodiments, R^{c} and R^{c′} are each independently isopropoxy. In some embodiments, R^{c} and R^{c′} are taken together with the carbon to which they are attached to form a carbonyl group (C(═O)).

In some embodiments, R^{c} and R^{c′} are taken together with the carbon to which they are attached to form a —C₃-C₉ cycloalkyl (e.g., cyclopropyl, cyclobutyl, cyclopentyl, cyclohexyl, cycloheptyl). In some embodiments, R^{c} and R^{c′} are taken together with the carbon to which they are attached to form a cyclopropyl. In some embodiments, R^{c} and R^{c′} are taken together with the carbon to which they are attached to form a cyclobutyl. In some embodiments, R^{c} and R^{c′} are taken together with the carbon to which they are attached to form a cyclopentyl. In some embodiments, R^{c} and R^{c′} are taken together with the carbon to which they are attached to form a cyclohexyl.

As generally defined herein, Ring A is selected from C₆-C₁₀ aryl, 5-10 membered heteroaryl and 3-10 membered heterocyclyl, substituted with 0, 1 or 2 instances of halo (e.g., —F) or -Me.

In some embodiments, Ring A is a C₆-C₁₀ aryl or a 3-10 membered heterocyclyl containing 1 or 2 heteroatoms selected from N, O and S, each substituted with 0, 1 or 2 instances of halo (e.g., —F) or -Me.

In some embodiments, Ring A is a C₆-C₁₀ aryl (e.g., phenyl, naphthyl) substituted with 0, 1 or 2 instances of halo (e.g., —F) or -Me. In further embodiments, Ring A is phenyl. In some embodiments, Ring A is unsubstituted phenyl. In some embodiments, ring A is phenyl substituted with one instance of halo (e.g., —F). In some embodiments, Ring A is phenyl substituted with one instance of —F. In some embodiments, ring A is phenyl substituted with 1 instance of -Me. In some embodiments, ring A is phenyl substituted with two instances of —F.

In some embodiments, ring A is phenyl substituted with two instances of -Me.

In other embodiments, Ring A a 3-10 membered heterocyclyl containing 1 or 2 heteroatoms selected from N, O and S (e.g., azetidinyl, oxetanyl, pyrolidinyl, tetrahydrofuranyl, piperidinyl, piperazinyl, morpholinyl, thiomorpholinyl, azepanyl, diazepanyl). In some embodiments, Ring A is selected from piperidinyl and piperazinyl. In some embodiments, ring A is piperidinyl. In some embodiments, ring A is piperazinyl.

In some embodiments, the moiety represented by

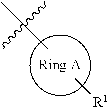

is selected from

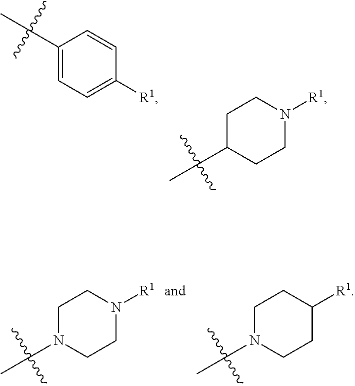

In certain embodiments, the moiety represented by

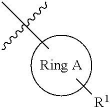

is

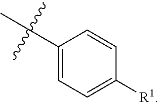

As generally defined herein, R¹ is an optionally substituted 5-10 membered heteroaryl.

In certain embodiments, R¹ is a 5-10 membered heteroaryl substituted with 0, 1 or 2 instances of R⁵

In some embodiments, R¹ is an optionally substituted 5-6 member monocyclic heteroaryl containing 1-3 heteroatoms selected from O, N and S. In some embodiments, R¹ is substituted with 0, 1 or 2 instances of R⁵, wherein R⁵ is as defined herein. In some embodiments, R¹ is unsubstituted. In some embodiments, R¹ is substituted with 1 instance of R³. In some embodiments, R¹ is substituted with 2 instances of R⁵.

In some embodiments, R¹ is a 6 member monocyclic heteroaryl containing 1-3 nitrogen atoms. In some embodiments, R¹ is selected from pyridinyl, pyrimidinyl, pyrazinyl, pyridazinyl and triazinyl each substituted with 0, 1 or 2 instances of R⁵. In some embodiments, R¹ is unsubstituted. In some embodiments, R¹ is substituted with 1 instance of R⁵. In some embodiments, R¹ is substituted with 2 instances of R⁵.

In certain embodiments, R¹ is a 5 member monocyclic heteroaryl containing 1-3 heteroatoms selected from O, N and S. In further embodiments, R¹ is selected from pyrollyl, pyrazolyl, imidazolyl, thiazolyl, furanyl, thiophenyl, oxazolyl, thiadiazolyl, oxadiazolyl, each substituted with 0, 1 or 2 instances of R⁵. In some embodiments, R¹ is pyrollyl. In some embodiments, R¹ is pyrazolyl. In some embodiments, R¹ is imidazolyl. In some embodiments, R¹ is thiazolyl. In some embodiments, R¹ is furanyl. In some embodiments, R¹ is thiophcnyl. In some embodiments, R¹ is oxazolyl. In some embodiments, R¹ is thiadiazolyl. In some embodiments, R¹ is oxadiazolyl. In In some embodiments, R¹ is unsubstituted. In some embodiments, R¹ is substituted with 1 instance of R⁵. In some embodiments, R¹ is substituted with 2 instances of R⁵.

In some embodiments, R¹ is selected from pyrazolyl and imidazolyl, each substituted with 0, 1 or 2 instances of R⁵.

In some embodiments, R¹ is pyrazolyl (e.g., pyrazol-1-yl) substituted with 0, 1 or 2 instances of R⁵. In some embodiments, R¹ is unsubstituted pyrazolyl (e.g., pyrazol-1-yl). In some embodiments, R¹ is pyrazolyl (e.g., pyrazol-1-yl) substituted with 1 instance of R⁵. In some embodiments, R¹ is pyrazolyl (e.g., pyrazol-1-yl) substituted with 2 instances of R⁵.

In some embodiments, R¹ is imidazolyl (e.g., imidazol-2-yl) substituted with 0, 1 or 2 instances of R⁵. In some embodiments, R¹ is unsubstituted imidazolyl (e.g., imidazol-2-yl). In some embodiments, R¹ is imidazolyl (e.g., imidazol-2-yl) substituted with one instance of R⁵. In some embodiments, R¹ is imidazolyl (e.g., imidazol-2-yl) substituted with 2 instances of R⁵.

As generally defined herein, each R⁵ is independently selected from halo, —CN, —C₁-C₆ alkyl including deuterated versions thereof, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R¹ and —S(═O)₂N(R^{a5})₂ wherein each alkyl, cycloalkyl, heterocyclyl, aryl, heteroaryl, arylalkyl and heteroarylalkyl is optionally substituted at any available position.

In certain embodiments, R⁵ is selected from CN, —C₁-C₆ alkyl (e.g., -Me, -CD₃, -Et, —Pr, -ⁱPr, -ⁿBu, -^{t}Bu), —C₁-C₆ haloalkyl (e.g., —CF₃, —CHF₂, —CH₂CF₃), —O(C₁-C₆ alkyl) (e.g., —OMe, —OEt), —C₃-C₁₀ cycloalkyl (e.g., cyclopropyl, cyclobutyl, cyclopentyl, cyclohexyl, cycloheptyl) and 3-10 membered heterocyclyl (e.g., azetidinyl, oxetanyl, tetrahydrofuranyl, tetrahydropyranyl, piperazinyl, piperidinyl, morpholinyl), wherein each alkyl, cycloalkyl and heterocyclyl is substituted with 0, 1 or 2 instances of-Me, —OMe, —OH, CN, halo (e.g., —F, —Cl). In certain embodiments, R⁵ is selected from CN, -Me, -CD₃, -Et, -ⁱPr, —CF₃, —OMe, —OEt, cyclopropyl, oxetanyl (e.g., oxetan-3-yl) and azetidinyl (e.g., N-methyl-azetidin-3-yl).

In certain embodiments, R⁵ is halo (e.g., fluoro, chloro, bromo, iodo). In further embodiments, R⁵ is —Cl. In some embodiments, R⁵ is —F. In some embodiments, R⁵ is —Br. In some embodiments, R⁵ is —I.

In some embodiments, R⁵ is —CN.

In certain embodiments, R⁵ is —C₁-C₆ alkyl (e.g., -Me, -Et, —Pr, -ⁱPr, -ⁿBu, -sec-Bu, -iso-Bu, -^{t}Bu). In further embodiments, R⁵ is -Me. In some embodiments, R⁵ is -Et. In some embodiments R⁵ is —Pr. In some embodiments, R⁵ is -iPr. In some embodiments, one or more hydrogens of the alkyl group are replaced with deuterium. In some embodiments, R⁵ is -CD₃.

In some embodiments, R⁵ is —C₁-C₆ heteroalkyl. In further embodiments, R⁵ is methoxymethyl (—CH₂OCH₃). In some embodiments, R⁵ is hydroxymethyl (—CH₂OH). In some embodiments, R⁵ is aminomethyl (e.g., —CH₂NH₂, —CH₂NHCH₃, —CH₂N(CH₃)₂. In some embodiments, R⁵ is —CH₂N(CH₃)CH₂CH₃.

In some embodiments, R⁵ is —C₁-C₆ haloalkyl. In further embodiments, R⁵ is trifluoromethyl (—CF₃). In other embodiments, R⁵ is difluoromethyl (—CHF₂).

In some embodiments, R⁵ is —O(C₁-C₆ alkyl) (e.g., methoxy, ethoxy, propoxy, isopropoxy). In some embodiments, R⁵ is selected from methoxy and ethoxy. In some embodiments, R⁵ is methoxy. In some embodiments, R⁵ is ethoxy. In some embodiments, R⁵ is propoxy. In some embodiments, R⁵ is isopropoxy.

In some embodiments, R⁵ is —C₁-C₆ hydroxyalkyl (e.g., —CH₂OH, —CH₂CH₂OH).

In some embodiments, R⁵ is —C₃-C₁₀ cycloalkyl (e.g., cyclopropyl, cyclobutyl, cyclopentyl, cyclohexyl). In some embodiments, R⁵ is cyclopropyl. In some embodiments R⁵ is cyclobutyl. In some embodiments, R⁵ is cyclopentyl. In some embodiments, R⁵ is cyclohexyl.

In some embodiments, R⁵ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3.3]heptanyl, 6-oxa-1-azaspiro[3.4]octanyl). In some embodiments, R⁵ is oxetanyl (e.g., oxetan-3-yl). In some embodiments, R⁵ is tetrahydropyranyl.

In some embodiments, R⁵ is tetrahydrofuranyl. In some embodiments, R⁵ is azetidinyl (e.g., N-methyl-azedidin-3-yl). In some embodiments, R⁵ is pyrrolidinyl. In some embodiments, R⁵ is piperidinyl. In some embodiments, R⁵ is piperazinyl. In some embodiments, R⁵ is morpholinyl.

In some embodiments, R⁵ is azepanyl. In some embodiments, R⁵ is 6-oxa-1-azaspiro[3.3]heptanyl. In some embodiments, R⁶ is 6-oxa-1-azaspiro[3.4]octanyl.

In some embodiments R³ is cycloalkylalkyl (e.g., cyclopropylmethyl, cyclobutylmethyl, cyclopentylmethyl, cyclohexylmethyl, cycloheptylmethyl). In some embodiments, R⁵ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R⁵ is arylalkyl. In some embodiments, R⁵ is benzyl.

In some embodiments, R⁵ is heteroarylalkyl (e.g., pyridinylmethyl, thiazolylmethyl, triazolylmethyl, pyrazolylmethyl).

In some embodiments, R⁵ is —OR⁵ (e.g., hydroxy (—OH), methoxy, difluoromethoxy (—OCHF₂), trifluoromethoxy (—OCF₃), —OCH(CH₃)CF₃, —OCH₂CF₃, ethoxy, propoxy, isopropoxy, cyclopropyloxy, cyclobutyloxy,). In some embodiments, R⁵ is hydroxy. In some embodiments, R⁵ is methoxy. In some embodiments, R⁵ is ethoxy. In some embodiments, R³ is propoxy. In some embodiments, R⁵ is isopropoxy. In some embodiments R⁵ is difluoromethoxy. (—OCHF₂). In some embodiments, R⁵ is trifluoromethoxy (—OCF₃). In some embodiments, R⁵ is —OCH(CH₃)CF₃. In some embodiments, R⁵ is —OCH₂CF₃. In some embodiments, R⁵ is cyclopropyloxy.

In some embodiments, R⁵ is N(R^{a5})₂ (e.g., —NH₂, —NHR^{a5}, —N(CH₃)R^{a5}). In some embodiments, R⁵ is —NH₂. In some embodiments, R⁵ is —NHR^{a5} (e.g., —NHMe, —NHEt, —NHPr, —NHⁱPr, —NHcyclopropyl, —NHcyclobutyl). In some embodiments, R⁵ is —N(CH₃)R^{a5} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R⁵ is —C(═O)R^{a5} or —C(═O)OR^{a5}. In some embodiments, R⁵ is —C(═O)R^{a5} wherein R is as described herein. In some embodiments, R⁵ is —C(═O)alkyl. In some embodiments, R⁵ is —C(O)CH₃, —C(O)cyclopropyl, —C(O)cyclobutyl, —C(O)Bu, —C(O)ⁱPr, —C(O)Pr, —C(O)^{t}Bu, or —C(═O)OMe. In some embodiments, R⁵ is acetyl (—C(═O)Me). In some embodiments, R⁵ is —C(═O)OR^{a5}. In some embodiments, R⁵ is —COOH. In some embodiments, R⁵ is COOMe.

In some embodiments, R⁵ is —NR^{a5}C(═O)R^{a5}. In certain embodiments, R⁵ is —NHC(═O)R^{a5} (e.g., NHC(═O)Me, NHC(═O)Et, NHC(═O)Pr, NHC(═O)ⁱPr, NHC(═O)Bu, NHC(═O)^{t}Bu, NHC(═O)Cyclopropyl, NHC(═O)Cyclobutyl). In some embodiments, R⁵ is —N(CH₃)C(═O)R^{a5} (e.g., N(CH₃)C(═O)Me, N(CH₃)C(═O)Et, N(CH₃)C(═O)Pr, N(CH₃)C(═O)ⁱPr, N(CH₃)C(═O)Bu, N(CH₃)C(═O)^{t}Bu, N(CH₃)C(═O)Cyclopropyl, N(CH)C(═O)Cyclobutyl).

In some embodiments, R⁵ is —NR^{a5}C(═O)OR^{a5}. In certain embodiments, R⁵ is —NHC(═O)OR^{a5} (e.g., NHC(═O)OMe, NHC(═O)OEt, NHC(═O)OPr, NHC(═O)OⁱPr, NHC(═O)OBu, NHC(═O)O^{t}Bu, NHC(═O)OCyclopropyl, NHC(═O)OCyclobutyl). In some embodiments, R⁵ is —N(CH₃)C(═O)OR^{a5} (e.g., N(CH₃)C(═O)OMe, N(CH₃)C(═O)OEt, N(CH₃)C(═O)OPr, N(CH₃)C(═O)OⁱPr, N(CH₃)C(═O)OBu, N(CH₃)C(═O)O^{t}Bu, N(CH₃)C(═O)OCyclopropyl, N(CH₃)C(═O)OCyclobutyl).

In some embodiments, R⁵ is —C(═O)N(R^{a5})₂(e.g., —C(═O)NH₂, —C(═O)NHR^{a5}, —C(═O)N(CH₃)R^{a5}). In some embodiments, R⁵ is —C(═O)NH₂. In certain embodiments, R⁵ is —C(═O)NHR^{a5} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁵ is —C(═O)N(CH₃)R^{a5} (e.g., —C(═O)NMe₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁵ is —OC(═O)N(R^{a5})₂. In certain embodiments, R⁵ is —OC(═O)NHR^{a5} (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁵ is —OC(═O)N(CH₃)R^{a5} (e.g., —OC(═O)NMe₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁵ is —S(═O)R^{a5}. In certain embodiments, R⁵ is —S(═O)alkyl (e.g., —S(═O)Me, —S(═O)Et, —S(═O)Pr, —S(═O)ⁱPr). In certain embodiments, R⁵ is —S(═O)cycloalkyl (e.g., —S(═O)cyclopropyl, —S(═O)cyclobutyl, —S(═O)cyclopentyl, —S(═O)cyclohexyl).

In some embodiments, R⁵ is —S(═O)₂R^{a5}. In certain embodiments, R⁵ is —S(═O)₂alkyl (e.g., —S(═O)₂Me, —S(═O)₂Et, —S(═O)₂Pr, —S(═O)₂ⁱPr). In certain embodiments, R⁵ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁵ is S(═O)₂aryl (e.g., S(═O)₂phenyl).

In some embodiments, R⁵ is —SR^{a5}. In certain embodiments, R⁵ is —Salkyl (e.g., —SMe, —SEt, —SPr, —SⁱPr). In certain embodiments, R⁵ is —Scycloalkyl (e.g., —Scyclopropyl, —Scyclobutyl, —Scyclopentyl, —Scyclohexyl). In certain embodiments, R⁵ is —Saryl (e.g., Sphenyl).

In some embodiments, R⁵ is —S(═O)(═NR^{a5})R^{a5}. In certain embodiments, R⁵ is —S(═O)(═NH)R^{a5} (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁵ is —S(═O)(═NCH₃)R^{a5} (e.g., —S(═O)(═NCH₃)Me, —S(═O)(═NCH₃)Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁵ is —NR^{a5} S(═O)₂R^{a5}. In certain embodiments, R⁵ is —NHS(═O)₂alkyl (e.g., —NHS(═O)₂Me, —NHS(═O)₂Et, —NHS(═O)₂Pr, —NHS(═O)₂ⁱPr). In certain embodiments, R⁵ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁵ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂ⁱPr). In certain embodiments, R⁵ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁵ is —S(═O)₂N(R^{a5})₂. (e.g., —S(═O)₂NH₂, —S(═O)₂NHR^{a5}, —S(═O)₂N(CH₃)R^{a5}). In some embodiments, R⁵ is —S(═O)₂NH₂. In some embodiments, R⁵ is —S(═O)₂NHR^{a5} (e.g., —S(═O)₂NHMe, —S(═O)₂NHEt, —S(═O)₂NHPr, —S(═O)₂NHⁱPr, —S(═O)₂NHcyclopropyl, —S(═O)₂NHcyclobutyl). In some embodiments, R⁵ is —S(═O)₂N(CH₃)R^{a5} (e.g., —S(═O)₂NMe₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R^{a5} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R is independently selected from H, —C₁-C₆ alkyl (e.g., -Me, -Et, —Pr, -ⁱPr, -ⁿBu, -^{t}Bu, -sec-Bu, -iso-Bu) and —C₁-C₆ haloalkyl (e.g., —CHF₂, —CF₃, —CH(CH₃)CF₃, —CH₂CF₃).

In some embodiments, each R^{a5} is independently H.

In some embodiments, each R^{a5} is independently —C₁-C₆ alkyl (e.g., -Me, -Et, —Pr, -ⁱPr, -ⁿBu, -^{t}Bu, -sec-Bu, -iso-Bu). In some embodiments, each R^{a5} is independently -Me. In some embodiments, each R^{a5} is independently -Et. In some embodiments, each R^{a5} is independently —Pr. In some embodiments, each R^{a5} is independently -ⁱPr.

In some embodiments, each R^{a5} is independently —C₁-C₆ heteroalkyl. In further embodiments, each R^{a5} is independently methoxymethyl (—CH₂OCH₃). In some embodiments, each RV is independently hydroxymethyl (—CH₂OH). In some embodiments, each RV is independently is aminomethyl (e.g., —CH₂NH₂, —CH₂NHCH₃, —CH₂N(CH)₂.

In some embodiments, each R^{a5} is independently —C₁-C₆ haloalkyl. In further embodiments, each RV is independently trifluoromethyl (—CF₃). In other embodiments, each R^{a5} is independently difluoromethyl (—CHF₂). In some embodiments, each R^{a5} is —CH(CH₃)CF₃. In some embodiments, each RV is —CH₂CF₃.

In some embodiments, each R^{a5} is independently —C₃-C₉ cycloalkyl (e.g., cyclopropyl, cyclobutyl, cyclopentyl, cyclohexyl). In some embodiments, each R^{a5} is independently cyclopropyl. In some embodiments each R^{a5} is independently cyclobutyl. In some embodiments, each R^{a5} is independently cyclopentyl. In some embodiments, each R^{a5} is independently cyclohexyl.

In some embodiments, each R^{a5} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{a5} is independently heteroaryl. In some embodiments, R^{a5} is independently a 5-10 member heteroaryl (e.g., a 5-6 member monocyclic heteroaryl or an 8-10 member bicyclic heteroaryl containing 1-3 heteroatoms independently selected from N, O and S). In some embodiments, R^{a5} is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{a5} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{a5} is independently thiophenyl (e.g., thiophen-2-yl, thiophen-3-yl). In some embodiments, R⁵ is independently pyrazolyl (e.g., pyrazol-1-yl, pyrazol-3-yl, pyrazol-5-yl). In some embodiments, R^{a5} is independently thiazolyl (e.g., thiazol-2-yl, thiazol-4-yl, thiazol-5-yl). In some embodiments, R^{a5} is independently a 6-member monocyclic heteroaryl (e.g., pyridyl, pyrimidinyl, triazinyl, pyrazinyl, pyridazinyl). In some embodiments, R^{a5} is independently pyridinyl (e.g., pyridin-2-yl, pyridin-3-yl, pyridin-4-yl). In some embodiments, R^{a5} is independently pyrimidinyl (e.g., pyrimidin-2-yl, pyrimidin-4-yl, pyrimidin-5-yl).

In some embodiments, R^{a5} is independently aryl. In some embodiments, R^{a5} is independently 6-10 member mono or bicyclic aryl. In some embodiments, R^{a5} is independently phenyl.

In some embodiments each R^{a5} is independently cycloalkylalkyl (e.g., cyclopropylmethyl, cyclobutylmethyl, cyclopentylmethyl, cyclohexylmethyl, cycloheptylmethyl). In some embodiments, each R^{a5} is independently heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, each R^{a5} is independently arylalkyl. In some embodiments, each R^{a5} is independently benzyl.

In some embodiments, each R^{a5} is independently heteroarylalkyl (e.g., pyridinylmethyl, thiazolylmethyl, triazolylmethyl, pyrazolylmethyl).

In some embodiments R¹ is selected from:

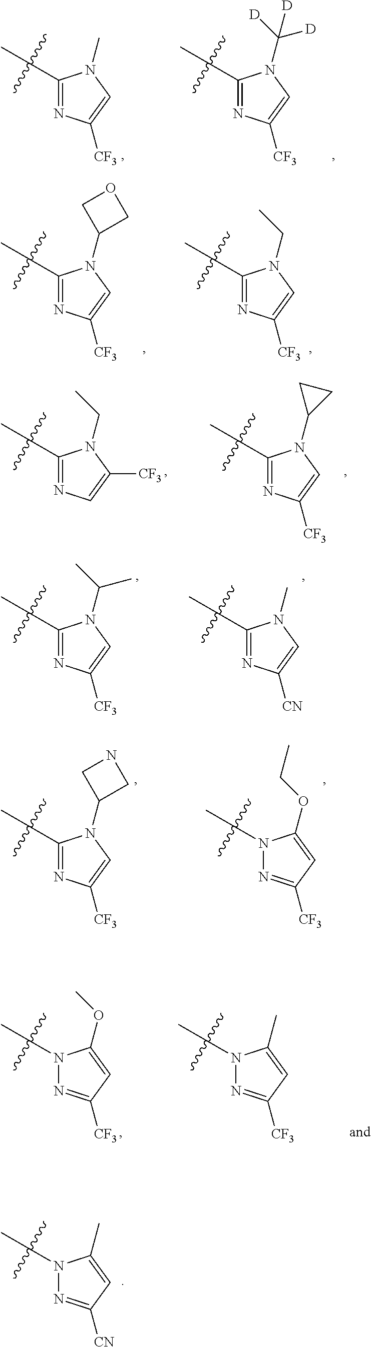

In some embodiments, R¹ is

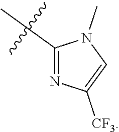 [SMILES: *c1nc(C(F)(F)F)cn1C]

In some embodiments, R¹ is

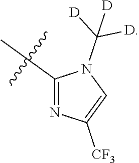

In some embodiments, R¹ is

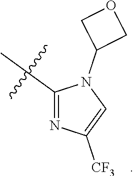 [SMILES: *c1nc(C(F)(F)F)cn1C1COC1]

In some embodiments, R¹ is

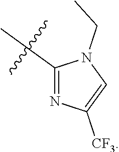 [SMILES: *c1nc(C(F)(F)F)cn1CC]

In some embodiments, R¹ is

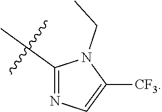 [SMILES: *c1ncc(C(F)(F)F)n1CC]

In some embodiments, R¹ is

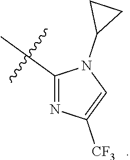 [SMILES: *c1nc(C(F)(F)F)cn1C1CC1]

In some embodiments, R¹ is

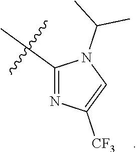 [SMILES: *c1nc(C(F)(F)F)cn1C(C)C]

In some embodiments, R¹ is

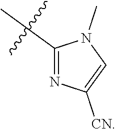 [SMILES: *c1nc(C#N)cn1C]

In some embodiments, R¹ is

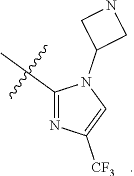 [SMILES: *c1nc(C(F)(F)F)cn1C1CNC1]

In some embodiments, R¹ is

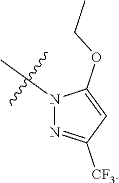 [SMILES: *n1nc(C(F)(F)F)cc1OCC]

In some embodiments, R¹ is

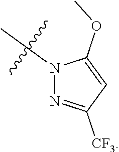 [SMILES: *n1nc(C(F)(F)F)cc1OC]

In some embodiments, R¹ is

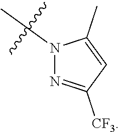 [SMILES: *n1nc(C(F)(F)F)cc1C]

In some embodiments, R¹ is

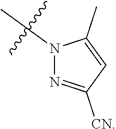 [SMILES: *n1nc(C#N)cc1C]

As generally defined herein, R² is absent or is selected from H, —C₁-C₆ alkyl, —C₃-C₉ cycloalkyl (e.g., cyclopropyl), —C₁-C₆ haloalkyl, —C₁-C₆ heteroalkyl, —C₁-C₆ hydroxyalkyl, arylalkyl, —S(═O)₂C₁-C₆alkyl (e.g., —S(═O)₂CH₃) and —C(═O)C₁-C₆alkyl (e.g., —C(═O)CH₃), wherein each hydrogen of the alkyl, heteroalkyl, hydroxylalkyl and arylalkyl can be independently replaced with a deuterium atom.

In some embodiments, R² is selected from H, -Me, -CD₃, -n-butyl, —CH₂CF₃, —S(═O)₂Me, —C(═O)Me, cyclopropyl, —CH₂CH₂OMe, —CH₂CH₂OH and benzyl.

In some embodiments, R² is absent.

In other embodiments, R² is selected from -Me, —CH₂CH₂OMe, —CH₂CH₂OH and benzyl.

In some embodiments, R² is H or -Me.

In certain embodiments, R² is —C₁-C₆ alkyl (e.g., -Me, -Et, —Pr, -ⁱPr, -ⁿBu, -sec-Bu, -iso-Bu, -^{t}Bu). In further embodiments, R² is -Me. In some embodiments, R² is -Et. In some embodiments R² is —Pr. In some embodiments, R² is -iPr.

In certain embodiments, R² is —C₁-C₆ alkyl (e.g., -Me, -Et, —Pr, -ⁱPr, -ⁿBu, -sec-Bu, -iso-Bu, -^{t}Bu) wherein one or more of the hydrogen atoms of the alkyl are replaced with a deuterium atom. (e.g., -CD₃, CD₂CD₃). In further embodiments, R² is -CD₃.

In some embodiments, R² is —C₁-C₆ heteroalkyl. In further embodiments, R² is methoxymethyl (—CH₂OCH₃). In some embodiments, R² is —CH₂CH₂OCH₃. In some embodiments, R² is hydroxymethyl (—CH₂OH). In some embodiments, R² is aminomethyl (e.g., —CH₂NH₂, —CH₂NHCH₃, —CH₂N(CH₃)₂. In some embodiments, R² is —CH₂N(CH₃)CH₂CH₃.

In some embodiments, R² is —C₁-C₆ haloalkyl. In further embodiments, R² is trifluoromethyl (—CF₃). In other embodiments, R² is difluoromethyl (—CHF₂). In some embodiments, R² is trifluoroethyl (—CH₂CF₃).

In some embodiments, R² is —C₁-C₆ hydroxyalkyl (e.g., —CH₂OH, —CH₂CH₂OH).

In some embodiments, R² is arylalkyl. In some embodiments, R² is benzyl.

In some embodiments, R² is —C(═O)C₁-C₆ alkyl. In some embodiments, R² is —C(O)CH₃, —C(O)^{t}Bu, —C(O)ⁱPr, —C(O)Pr, or C(O)Bu. In some embodiments, R² is acetyl (—C(═O)Me).

In certain embodiments, R² is —S(═O)₂C₃-C₆ alkyl (e.g., —S(═O)₂Me, —S(═O)₂Et, —S(═O)₂Pr, —S(═O)₂ⁱPr). In some embodiments, R² is —S(═O)₂Me.

In some embodiments, R² is —C₃-C₉ cycloalkyl (e.g., cyclopropyl, cyclobutyl, cyclopentyl, cyclohexyl). In some embodiments, R² is cyclopropyl. In some embodiments R³ is cyclobutyl. In some embodiments, R² is cyclopentyl. In some embodiments, R² is cyclohexyl.

As generally defined herein, R⁶ is selected from H, -D, —CN, halo, —C₁-C₆ alkyl, —C₁-C₆ heteroalkyl, —C₁-C₆ haloalkyl, —C₃-C₁₀ cycloalkyl, —OH, and —O(C₁-C₆ alkyl).

In certain embodiments, R⁶ is selected from H, D, —CN, halo (e.g., —F, —Cl), —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CF₃), —OH, and —O(C₁-C₆ alkyl) (e.g., —OMe).

In some embodiments, R⁶ is selected from H, -D, —CN, —F, —Cl, -Me, -Et, —Pr, -ⁱPr, -ⁿBu, -^{t}Bu, —CF₃, —CHF₂, —OH and —OMe). In further embodiments, R⁶ is selected from H, —F, Me and —OMe. In yet further embodiments, R⁶ is H.

In some embodiments, R⁶ is D.

In certain embodiments, R⁶ is halo (e.g., fluoro, chloro, bromo, iodo). In further embodiments, R⁶ is —Cl. In some embodiments, R⁶ is —F. In some embodiments, R⁶ is —Br. In some embodiments, R⁶ is —I.

In some embodiments, R⁶ is —CN.

In certain embodiments, R⁶ is —C₁-C₆ alkyl (e.g., -Me, -Et, —Pr, -ⁱPr, -ⁿBu, -sec-Bu, -iso-Bu, -^{t}Bu). In further embodiments, R⁶ is -Me. In some embodiments, R⁶ is -Et. In some embodiments R⁶ is —Pr. In some embodiments, R⁶ is -iPr.

In some embodiments, R⁶ is —C₁-C₆ heteroalkyl. In further embodiments, R⁶ is methoxymethyl (—CH₂OCH₃). In some embodiments, R⁶ is hydroxymethyl (—CH₂OH). In some embodiments, R⁶ is aminomethyl (e.g., —CH₂NH₂, —CH₂NHCH₃, —CH₂N(CH₃)₂. In some embodiments, R⁶ is —CH₂N(CH₃)CH₂CH₃.

In some embodiments, R⁶ is —C₁-C₆ haloalkyl. In further embodiments, R⁶ is trifluoromethyl (—CF₃). In other embodiments, R⁶ is difluoromethyl (—CHF₂).

In some embodiments, R⁶ is —C₃-C₁₀ cycloalkyl (e.g., cyclopropyl, cyclobutyl, cyclopentyl, cyclohexyl). In some embodiments, R⁶ is cyclopropyl. In some embodiments R⁶ is cyclobutyl. In some embodiments, R⁶ is cyclopentyl. In some embodiments, R⁶ is cyclohexyl.

In some embodiments, R⁶ is hydroxy (—OH). In certain embodiments, R⁶ is —O(C₁-C₆ alkyl) (e.g., methoxy, ethoxy, propoxy, isopropoxy). In some embodiments, R⁶ is methoxy. In some embodiments, R⁶ is ethoxy. In some embodiments, R⁶ is propoxy. In some embodiments, R⁶ is isopropoxy.

In some embodiments, the compound is selected from the compounds of Table 1.

In one embodiment, provided is a pharmaceutical composition comprising a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof as defined herein and a pharmaceutically acceptable carrier. In some embodiments, the pharmaceutical composition further comprises a second therapeutic agent.

In various embodiments, the Compounds of the Disclosure are USP1 inhibitors that reduce the level of USP1 protein and/or inhibit or reduce at least one biological activity of USP 1 protein.

In some embodiments, the Compounds of the Disclosure specifically bind to USP1 protein. In some embodiments, the Compounds of the Disclosure specifically bind to USP1 protein in a USP1-UAF1 complex. In some embodiments, the Compounds of the Disclosure specifically bind to USP1 mRNA. In some embodiments, the Compounds of the Disclosure specifically bind to USP1 protein (alone or in a USP1-UAF1 complex) or USP1 mRNA. In some embodiments, the Compounds of the Disclosure specifically bind to UAF1 (alone or in a USP1-UAF1 complex) and inhibit or reduces formation or activity of the USP1-UAF1 complex.

In some embodiments, the Compounds of the Disclosure decrease the formation of the USP1-UAF1 complex. In some embodiments, the Compounds of the Disclosure decrease the activity of the USP1-UAF1 complex. In some embodiments, the Compounds of the Disclosure decrease the deubiquitinase activity of USP1. In some embodiments, the Compounds of the Disclosure increase mono-ubiquitinated PCNA. In some embodiments, the Compounds of the Disclosure increase mono-ubiquitinated FANCD2. In some embodiments, the Compounds of the Disclosure increase mono-ubiquitinated FANCI.

In some embodiments, the Compounds of the Disclosure do not bind to other deubiquitinases, other USP proteins, or other UAF1 complexes (e.g., USP46-UAF1) or bind deubiquitinases, other USP proteins, or other UAF1 complexes (e.g., USP46-UAF1) with at least 5-fold, at least 10-fold, at least 20-fold, or at least 100-fold reduced affinity compared to the affinity for USP1 (i.e., the K_{D} of the USP1 inhibitor for other deubiquitinases, other USP proteins, or other UAF1 complexes (e.g., USP46-UAF1) is at least 5-fold, at least 10-fold, at at least 20 fold, least 50 fold, at least 100 fold.

Certain compounds of the disclosure were assessed for USP1-UAF1 activity in a Ubiquitin Rhodamine assay as described in Example 135.

Table 1 indicates IC₅₀ values (μM) against USP1-UAF1 for exemplary compounds (column 4). For column 4, “a” indicates an IC₅₀ value lower than 30 nM, “b” indicates an IC₅₀ value equal to or greater than 30 nM and lower than 100 nM, “c” indicates an IC₅₀ value equal to or greater than 100 nM but lower than 10 μM, and “d” indicates an IC₅₀ value equal to or greater than 10 μM.

Table 1 also indicates IC₅₀ values in a viability assay for a non-isogenic pair of BRCA1 mutant (column 5—MDA-MB-436) and BRCA1 WT (column 6—HCC1954) cell lines. These values indicate the effect of treatment with compound on cell survival. In columns 5 and 6, a value of “aa” and “aaa” indicates an IC₅₀ of less than 100 nM in the mutant and wild-type cell lines, respectively; a value of “bb” “bbb” indicates an IC₅₀ equal to or greater than 100 nM but less than 250 nM in the mutant and wild-type cell lines, respectively; a value of “cc” and “ccc” indicates an IC₅₀ equal to or greater than 250 nM but less than 10 μM in the mutant and wild-type cell lines, respectively; a value of “dd” and “ddd” indicates an IC₅₀ greater than or equal to 10 μM in the mutant and wild-type cell lines, respectively.

Table 1 also indicates IC₅₀ values for exemplary compounds in an AlphaLISA assay measuring monoubiquitinated PCNA in a BRCA1 mutant cell line (MDA-MB-436; column 7). In column 7, a value of “A” indicates an IC₅₀ of less than 100 nM, a value of “B” indicates an IC₅₀ equal to or greater than 100 nM but less than 250 nM, a value of “C” indicates an IC₅₀ equal to or greater than 250 nM but less than 10 μM, a value of “D” indicates an IC₅₀ greater than or equal to 10 μM.

Unless otherwise indicated, the absolute stereochemistry of all chiral atoms is as depicted. Compounds marked with (or) or (re) are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds marked with (abs) are single enantiomers wherein the absolute stereochemistry is as indicated.

TABLE 1
Exemplary compounds and biological data

| | | | | | HCC | |
|---|---|---|---|---|---|---|
| | | | | MDA- | 1954 | |
| | | | USP1- | MB- | IC50 | Alpha |
| | | Ster- | UAF1 | 436 rel | rel | LISA |
| Structure | Nr. | eo | IC50 | IC50 | IC50 | IC50 |
| 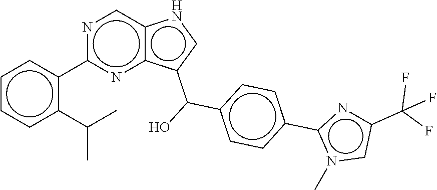 | 1 | | b | aa | ddd | A |
| 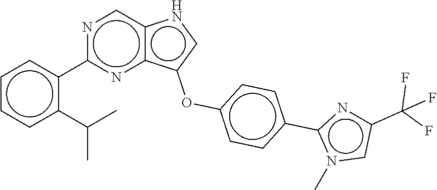 | 2 | | b | bb | ddd | A |
| 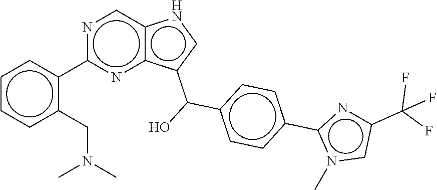 | 3 | | d | dd | ddd | |
| 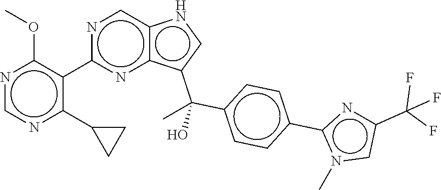 | 4 | or | b | bb | | B |
| 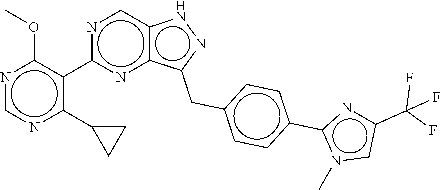 | 5 | | a | aa | | A |
| 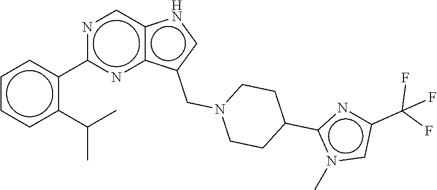 | 6 | | c | cc | ddd | |
| 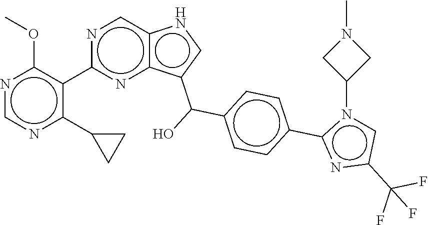 | 7 | | d | dd | ddd | |
| 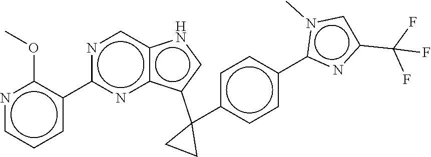 | 8 | | b | cc | ccc | B |
| 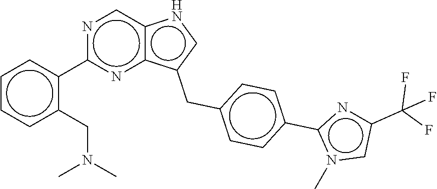 | 9 | | d | dd | ddd | |
| 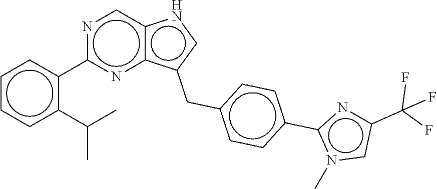 | 10 | | a | aa | ddd | A |
| 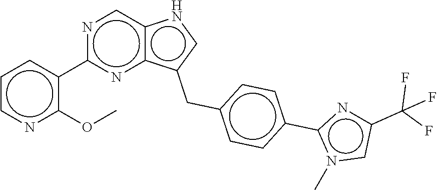 | 11 | | b | cc | ddd | B |
| 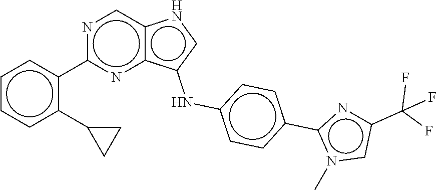 | 12 | | c | cc | ccc | C |
| 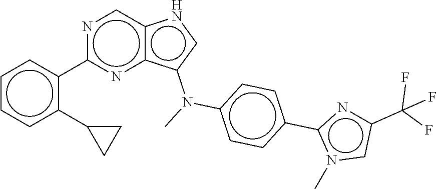 | 13 | | c | cc | ddd | |
| 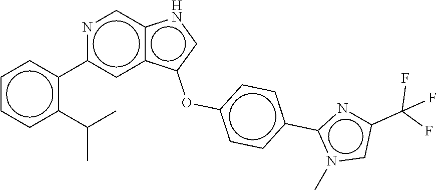 | 14 | | a | aa | ddd | A |
| 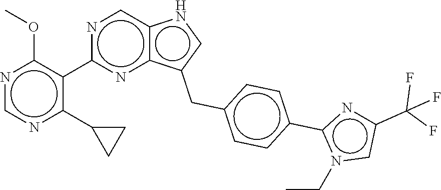 | 15 | | a | aa | ddd | B |
| 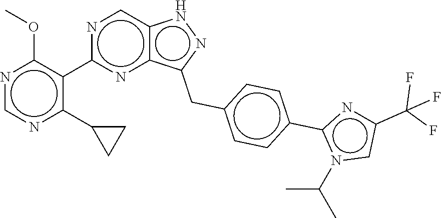 | 16 | | | aa | ddd | |
| 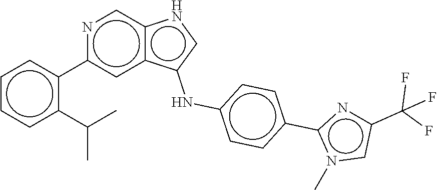 | 17 | | c | cc | ccc | A |
| 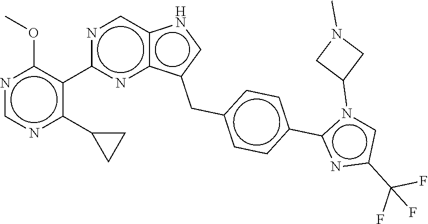 | 18 | | c | bb | ddd | B |
| 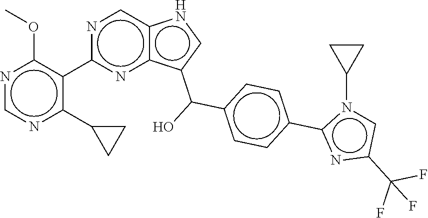 | 19 | | c | | | |
| 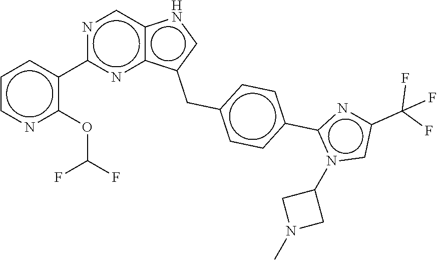 | 20 | | c | bb | ddd | A |
| 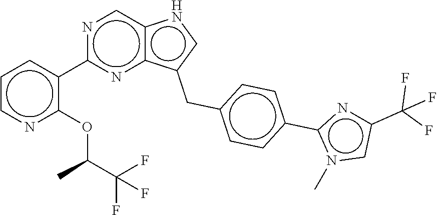 | 21 | or | a | aa | ccc | A |
| 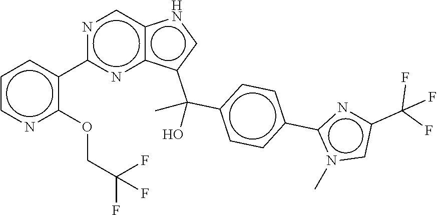 | 22 | | c | bb | ccc | |
| 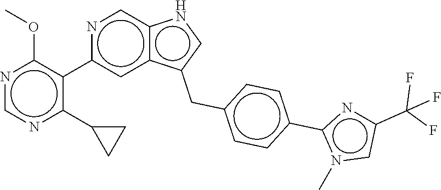 | 23 | | a | aa | ccc | A |
| 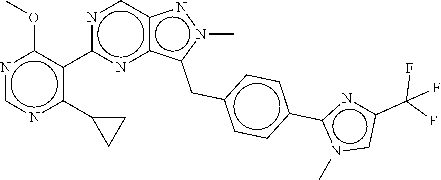 | 24 | | | cc | ddd | B |
| 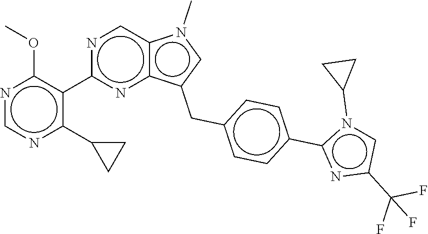 | 25 | | a | | | A |
| 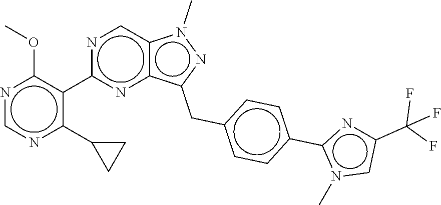 | 26 | | | aa | ddd | A |
| 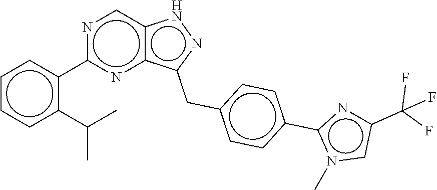 | 27 | | a | aa | ddd | A |
| 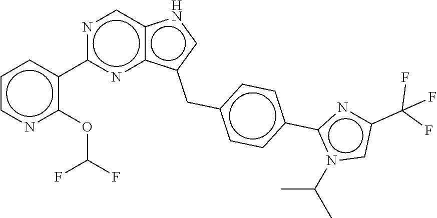 | 28 | | b | aa | ddd | B |
| 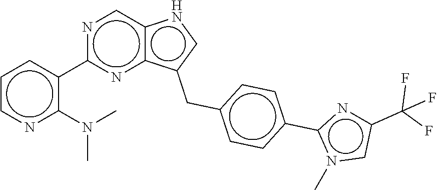 | 29 | | c | cc | ddd | C |
| 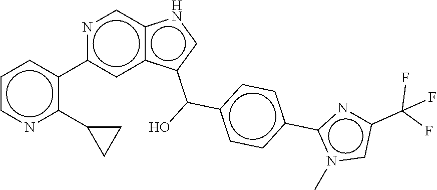 | 30 | | c | dd | ddd | |
| 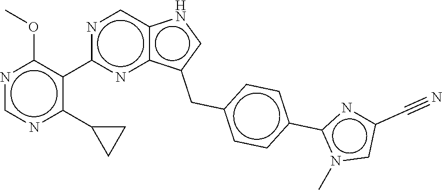 | 31 | | c | cc | ddd | C |
| 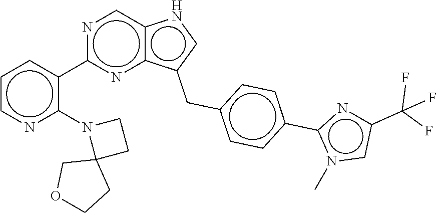 | 32 | | c | cc | ccc | |
| 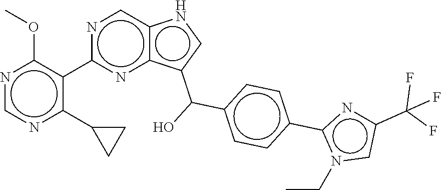 | 33 | | c | cc | ddd | C |
| 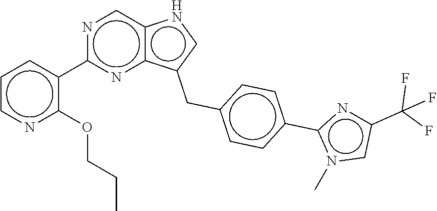 | 34 | | b | bb | ddd | B |
| 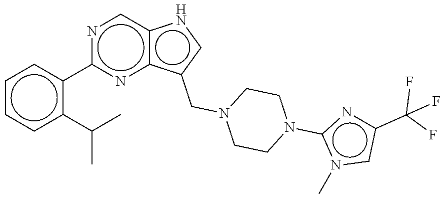 | 35 | | c | cc | ccc | |
| 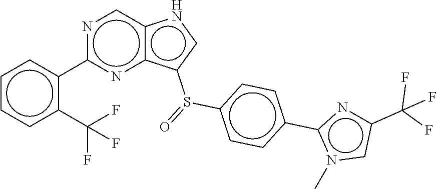 | 36 | | c | | | |
| 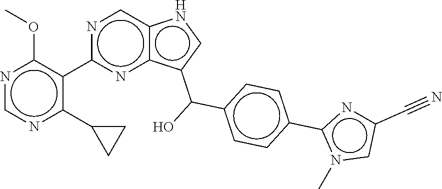 | 37 | | d | | | |
| 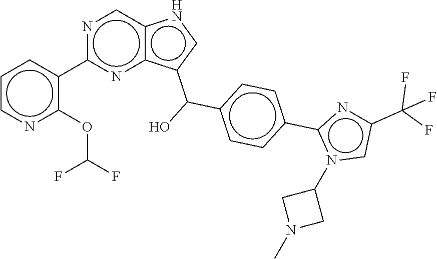 | 38 | | d | dd | ddd | |
| 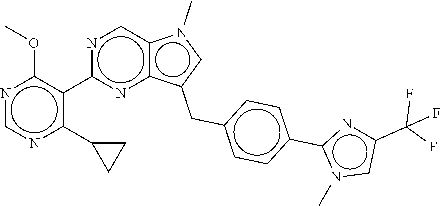 | 39 | | a | aa | ddd | |
| 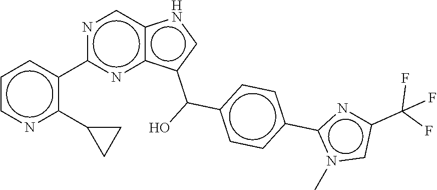 | 40 | | c | cc | ddd | |
| 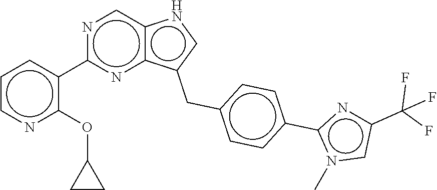 | 41 | | b | bb | ddd | A |
| 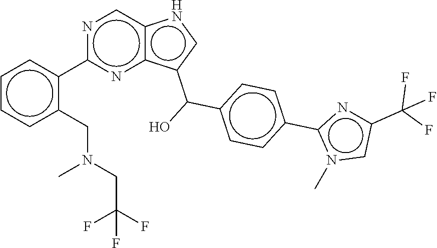 | 42 | | c | cc | ddd | |
| 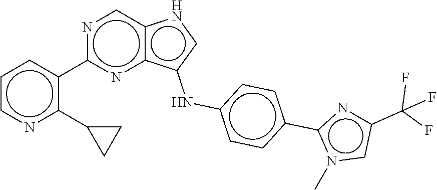 | 43 | | c | dd | ddd | |
| 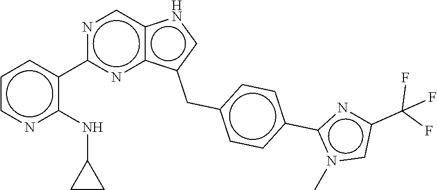 | 44 | | c | cc | ccc | |
| 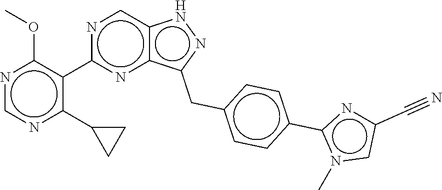 | 45 | | c | cc | ddd | C |
| 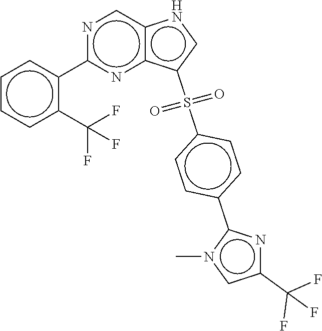 | 46 | | c | | | |
| 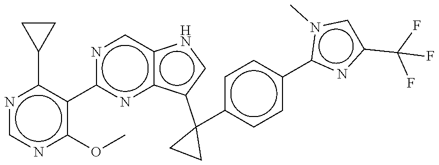 | 47 | | b | cc | ddd | B |
| 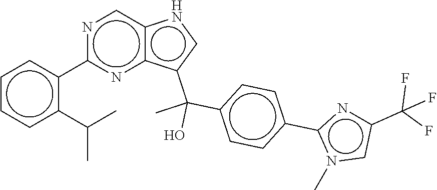 | 48 | | a | aa | ddd | A |
| 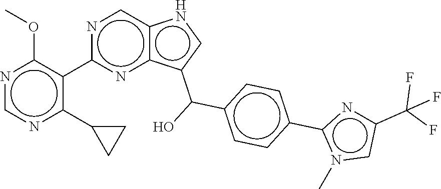 | 49 | | c | cc | ddd | |
| 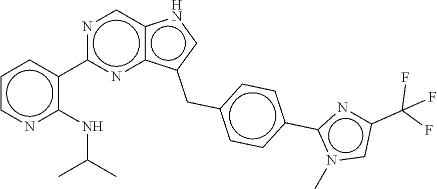 | 50 | | c | cc | ccc | |
| 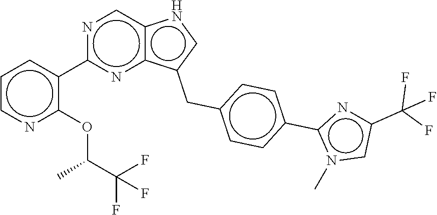 | 51 | or | a | aa | ccc | A |
| 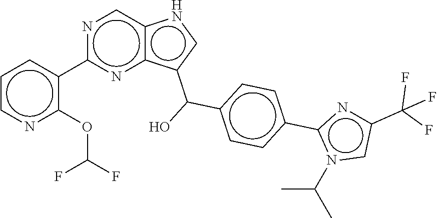 | 52 | | c | cc | ddd | |
| 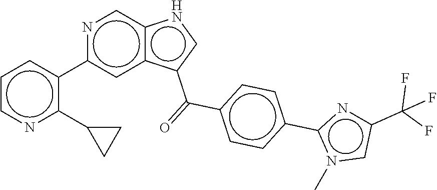 | 53 | | d | dd | ddd | |
| 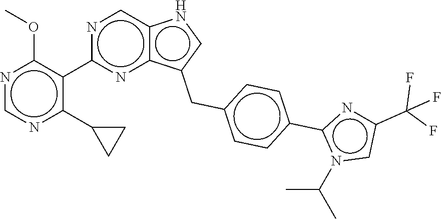 | 54 | | a | aa | | A |
| 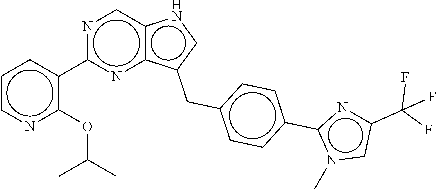 | 55 | | a | aa | ddd | |
| 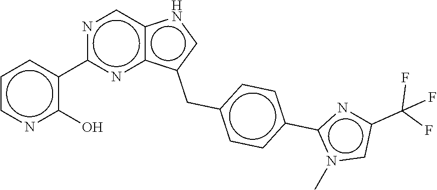 | 56 | | d | dd | ddd | |
| 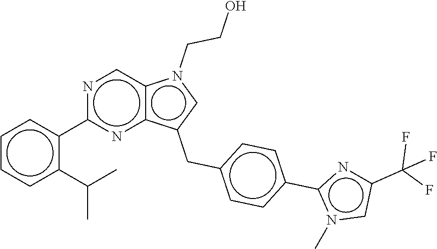 | 57 | | a | cc | ddd | B |
| 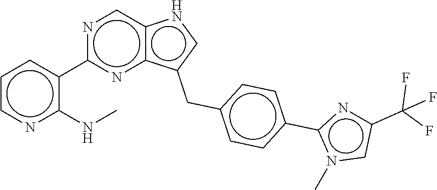 | 58 | | c | cc | ccc | |
| 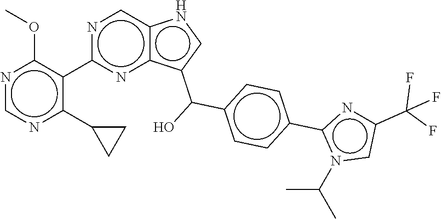 | 59 | | c | cc | ccc | |
| 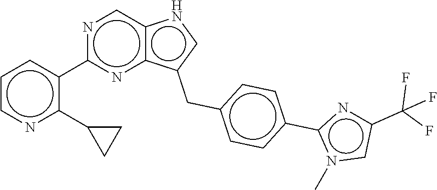 | 60 | | a | aa | ddd | A |
| 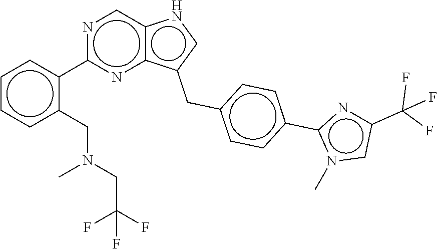 | 61 | | b | cc | ccc | |
| 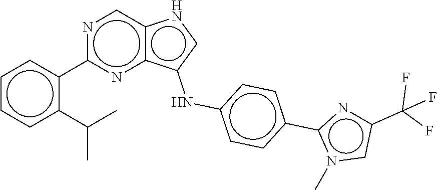 | 62 | | c | cc | ccc | |
| 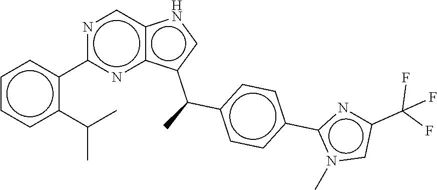 | 64 | or | a | aa | ccc | A |
| 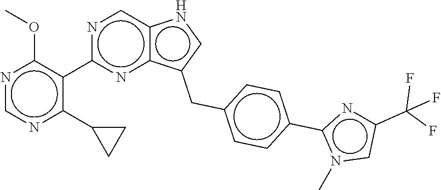 | 65 | | a | aa | ddd | A |
| 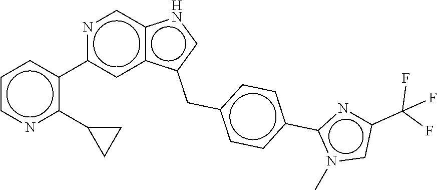 | 66 | | b | bb | ccc | A |
| 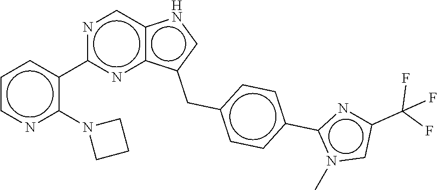 | 67 | | c | cc | ddd | C |
| 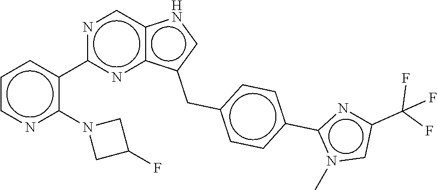 | 68 | | c | | | |
| 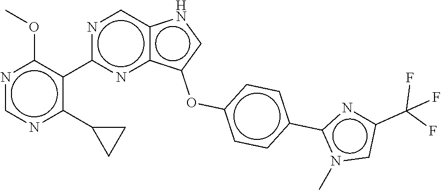 | 69 | | c | cc | ddd | C |
| 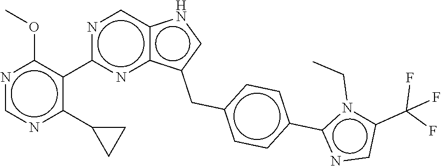 | 70 | | c | | | |
| 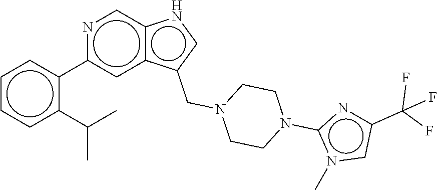 | 71 | | c | cc | ccc | C |
| 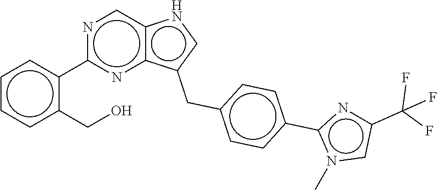 | 72 | | c | cc | ddd | |
| 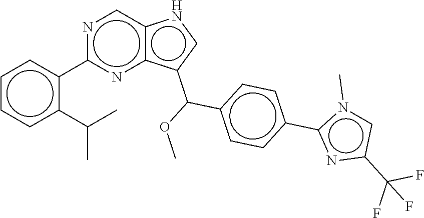 | 73 | | c | cc | ccc | B |
| 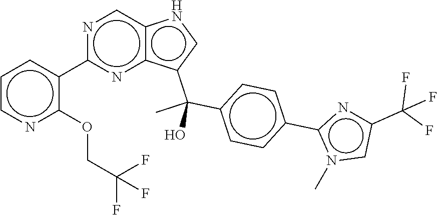 | 74 | or | a | cc | ccc | B |
| 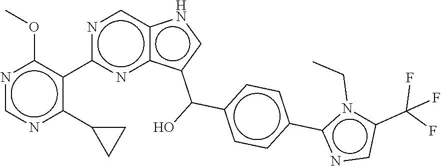 | 75 | | d | dd | ddd | |
| 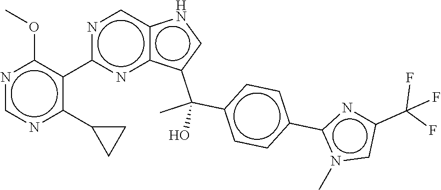 | 76 | or | c | cc | | B |
| 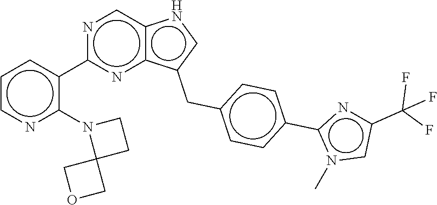 | 77 | | c | cc | ddd | |
| 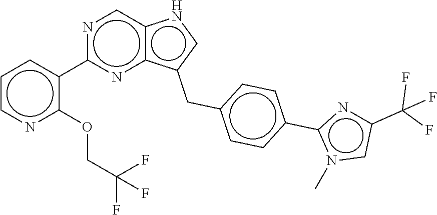 | 78 | | a | aa | ccc | A |
| 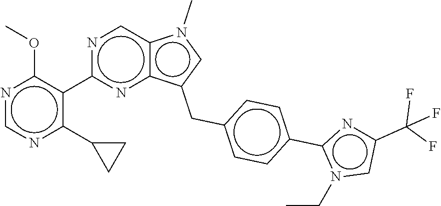 | 79 | | a | | | A |
| 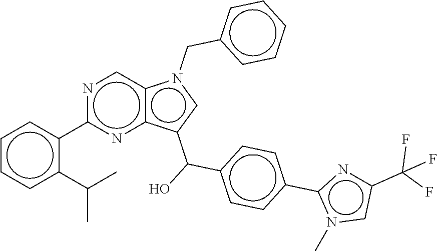 | 80 | | c | cc | ddd | |
| 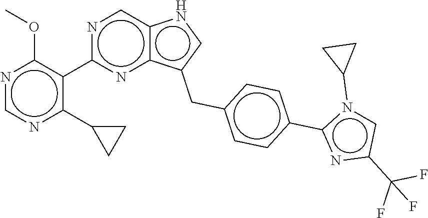 | 81 | | a | aa | ccc | A |
| 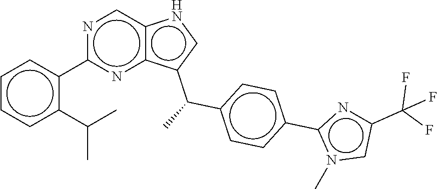 | 82 | or | a | aa | ccc | A |
| 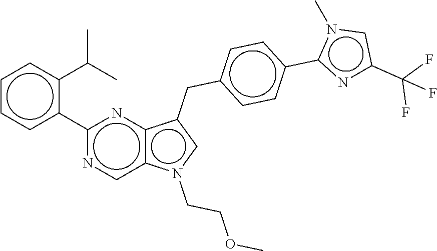 | 83 | | a | aa | ddd | A |
| 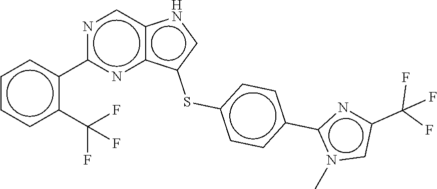 | 84 | | c | cc | ddd | C |
| 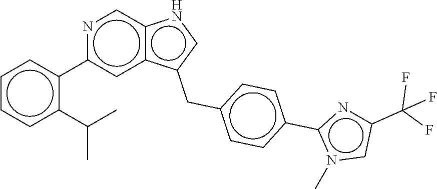 | 85 | | a | cc | ccc | B |
| 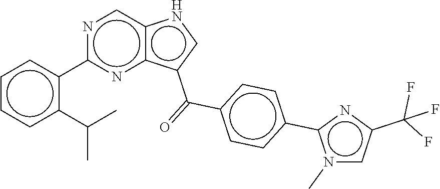 | 86 | | d | cc | ccc | |
| 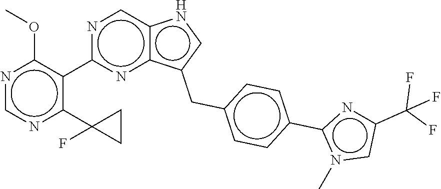 | 87 | | a | aa | ddd | A |
| 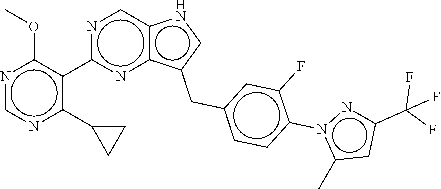 | 88 | | a | aa | ddd | A |
| 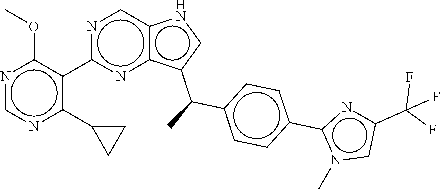 | 89 | or | a | aa | ccc | A |
| 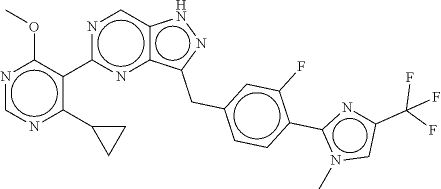 | 90 | | a | aa | ddd | A |
| 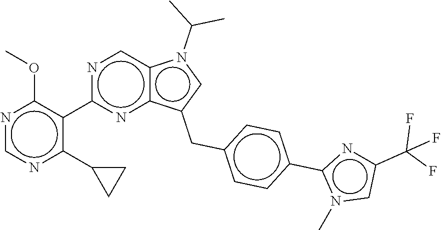 | 91 | | c | bb | ddd | B |
| 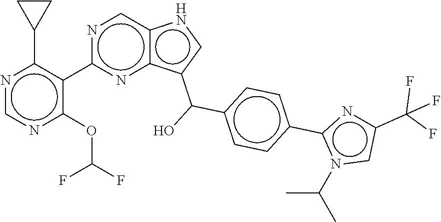 | 92 | | b | aa | dd | A |
| 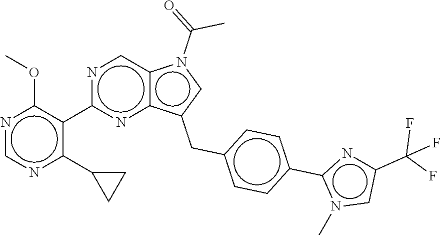 | 93 | | b | aa | ddd | A |
| 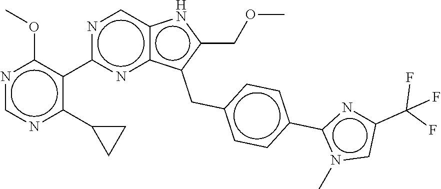 | 94 | | a | aa | ddd | A |
| 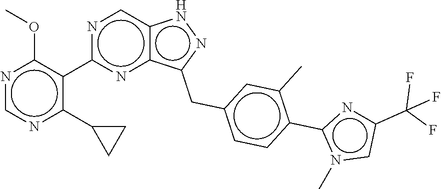 | 95 | | a | aa | ddd | A |
| 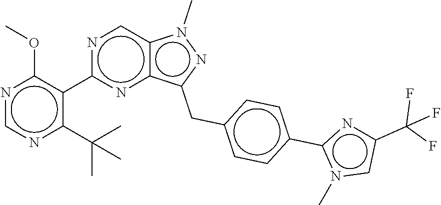 | 96 | | b | bb | ddd | B |
| 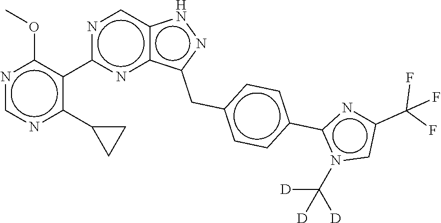 | 97 | | a | aa | ddd | A |
| 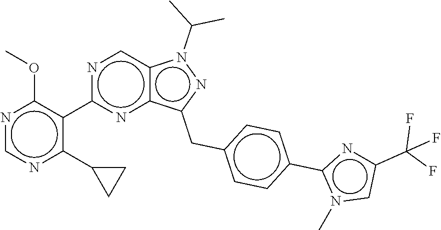 | 98 | | b | aa | ddd | A |
| 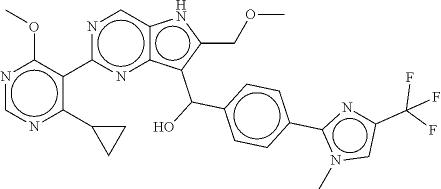 | 99 | | c | cc | ddd | |
| 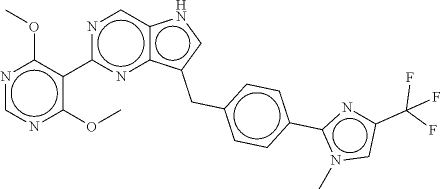 | 100 | | b | bb | ddd | A |
| 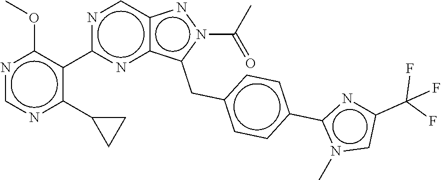 | 101 | | b | aa | ddd | A |
| 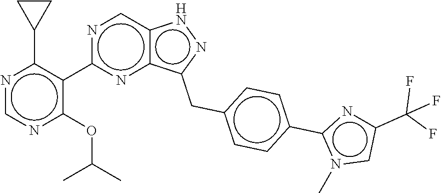 | 102 | | a | bb | ddd | B |
| 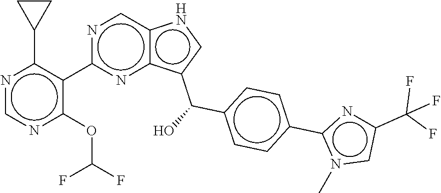 | 103 | or | b | aa | ddd | A |
| 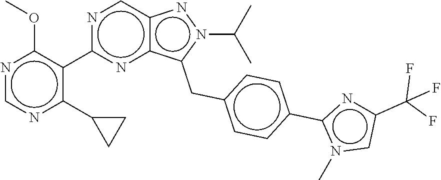 | 104 | | c | bb | ddd | B |
| 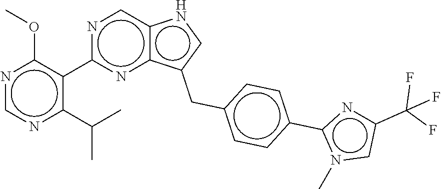 | 105 | | a | aa | ddd | A |
| 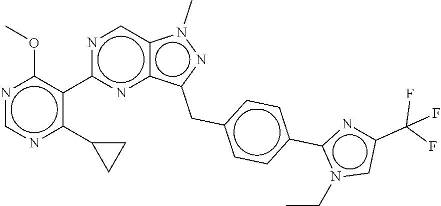 | 106 | | a | aa | ddd | A |
| 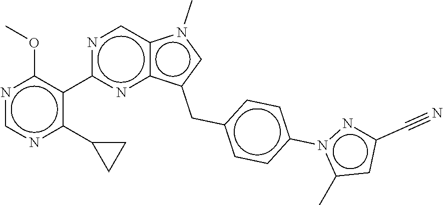 | 107 | | b | aa | ddd | A |
| 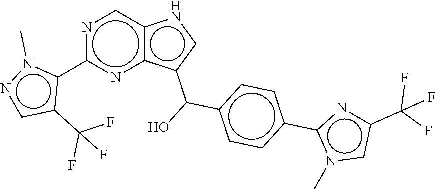 | 108 | | c | cc | ddd | |
| 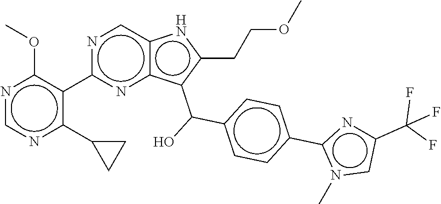 | 109 | | c | cc | ddd | |
| 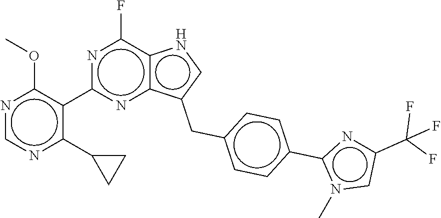 | 110 | | a | bb | ddd | C |
| 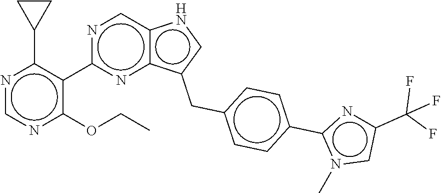 | 111 | | b | aa | ddd | A |
| 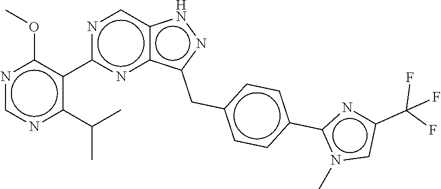 | 112 | | a | | | |
| 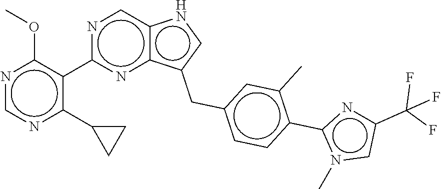 | 113 | | a | aa | ddd | A |
| 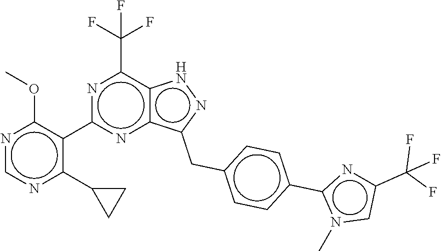 | 114 | | a | aa | ddd | A |
| 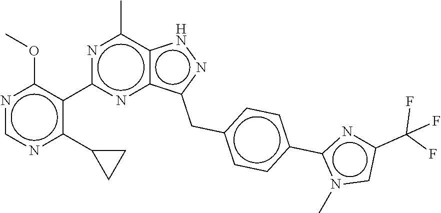 | 115 | | a | aa | ddd | A |
| 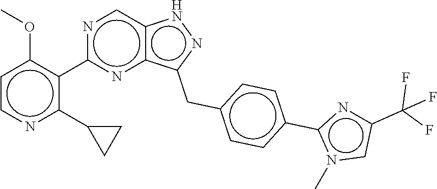 | 116 | | a | aa | ddd | B |
| 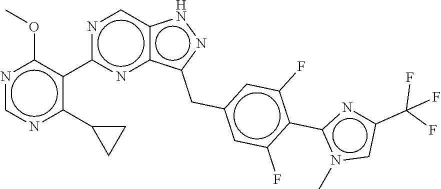 | 117 | | a | aa | ddd | A |
| 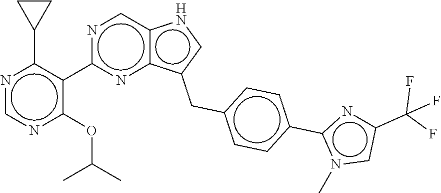 | 118 | | a | aa | ddd | A |
| 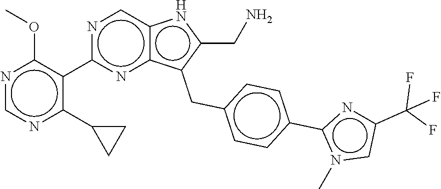 | 119 | | c | bb | ccc | C |
| 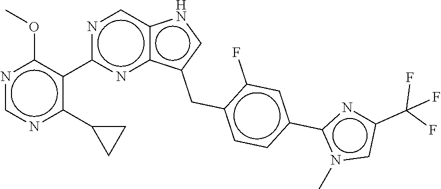 | 120 | | a | aa | ddd | A |
| 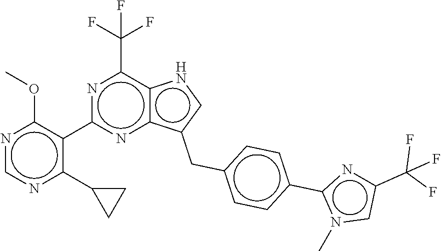 | 121 | | a | aa | ddd | A |
| 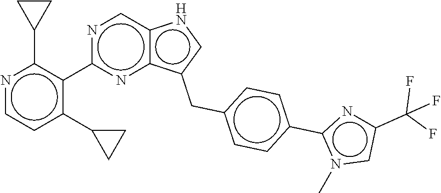 | 122 | | a | bb | ddd | C |
| 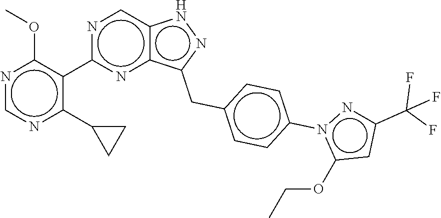 | 123 | | a | aa | ccc | A |
| 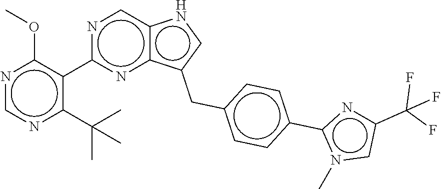 | 124 | | b | aa | ddd | B |
| 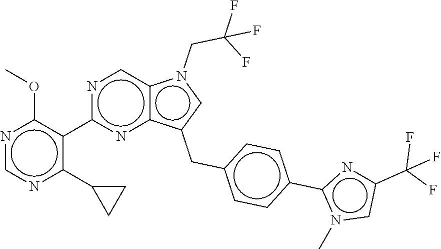 | 125 | | a | aa | ccc | A |
| 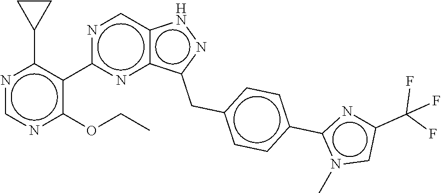 | 126 | | a | aa | ccc | A |
| 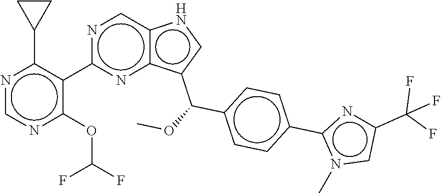 | 127 | or | c | | | |
| 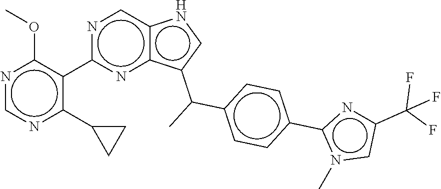 | 128 | | a | aa | ddd | |
| 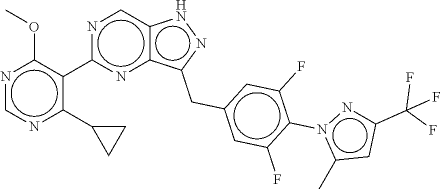 | 129 | | a | bb | ccc | B |
| 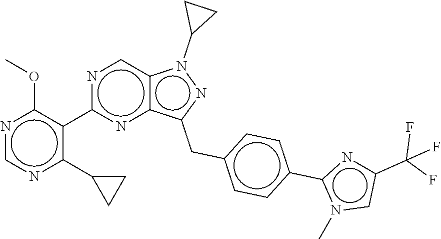 | 130 | | | | | |
| 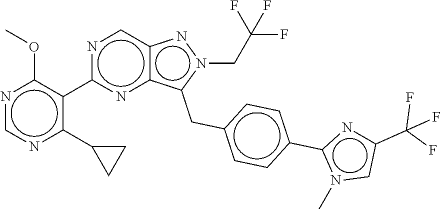 | 131 | | c | aa | ddd | A |
| 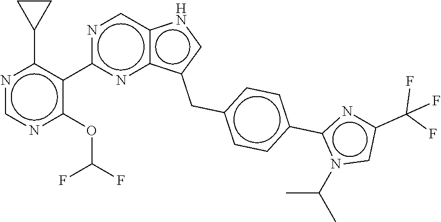 | 132 | | a | aa | ddd | A |
| 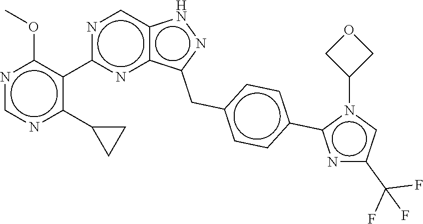 | 133 | | b | aa | ddd | B |
| 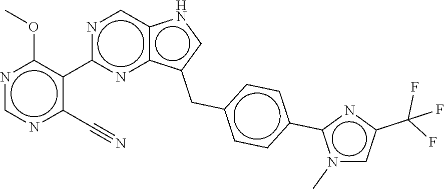 | 134 | | b | bb | ddd | C |
| 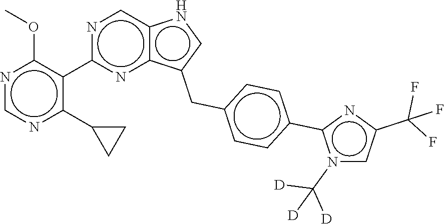 | 135 | | a | aa | ddd | A |
| 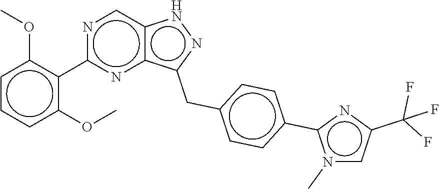 | 136 | | a | bb | ddd | C |
| 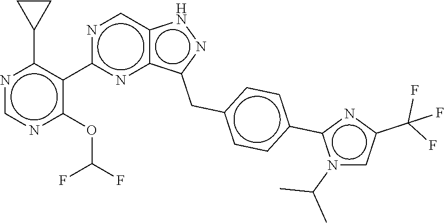 | 137 | | a | aa | ccc | A |
| 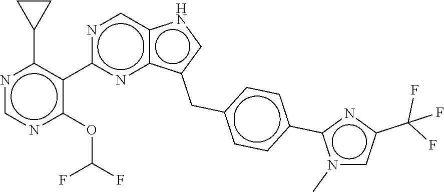 | 138 | | a | aa | ddd | A |
| 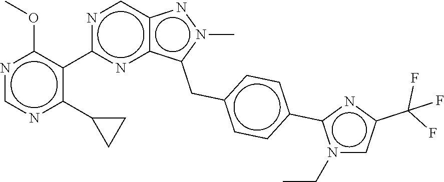 | 139 | | b | aa | ddd | A |
| 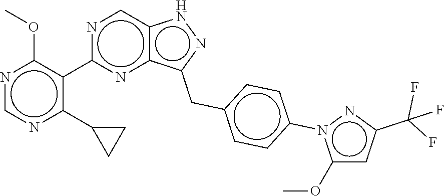 | 140 | | a | aa | ccc | B |
| 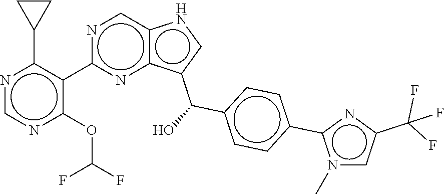 | 141 | or | a | aa | ddd | A |
| 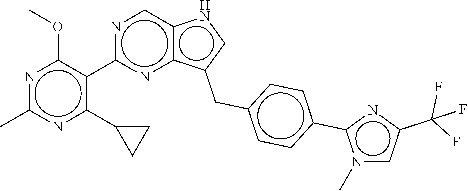 | 142 | | b | aa | ddd | A |
| 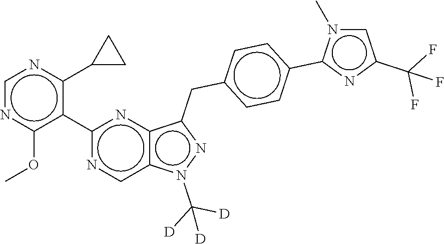 | 143 | | a | aa | ddd | A |
| 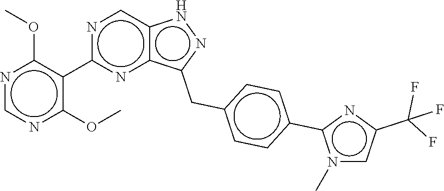 | 144 | | a | bb | ddd | B |
| 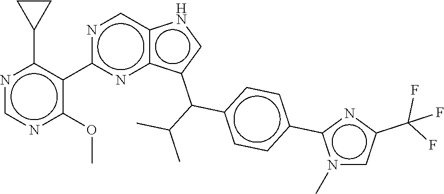 | 145 | | b | bb | ccc | |
| 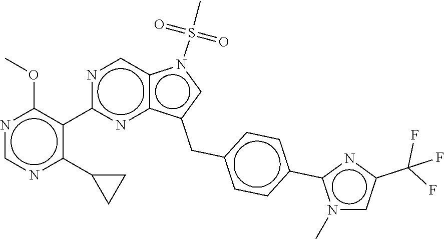 | 146 | | a | aa | ddd | B |
| 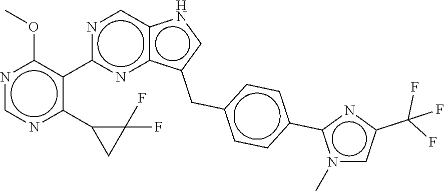 | 147 | | a | aa | ddd | A |
| 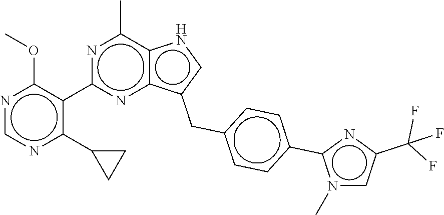 | 148 | | a | aa | ddd | A |
| 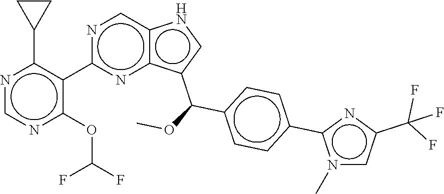 | 149 | or | a | | | |
| 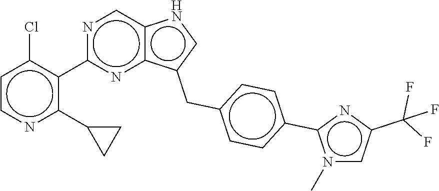 | 150 | | a | aa | ccc | A |
| 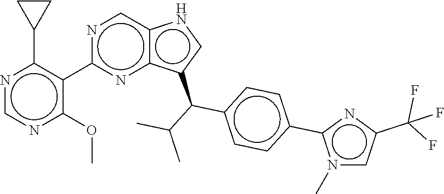 | 151 | or | b | bb | ccc | A |
| 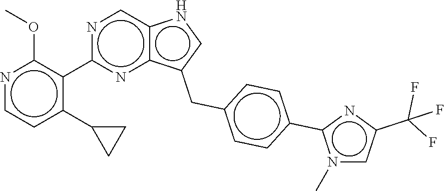 | 152 | | a | aa | ddd | A |
| 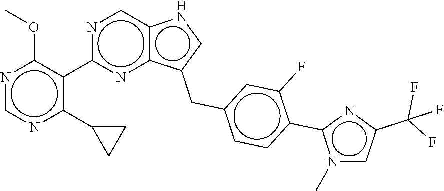 | 153 | | a | aa | ddd | A |
| 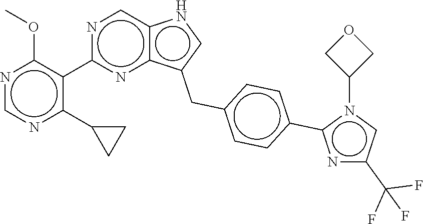 | 154 | | b | aa | ddd | B |
| 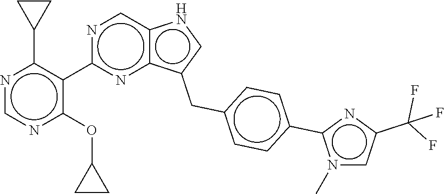 | 155 | | a | aa | ddd | |
| 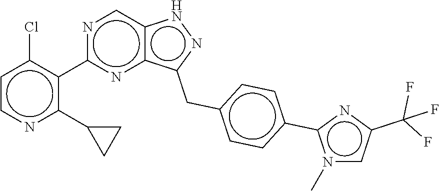 | 156 | | a | aa | ddd | |
| 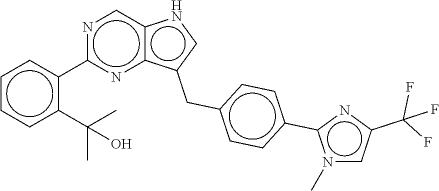 | 157 | | a | | | |
| 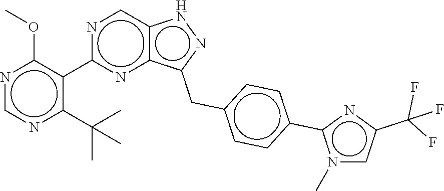 | 158 | | a | | | |
| 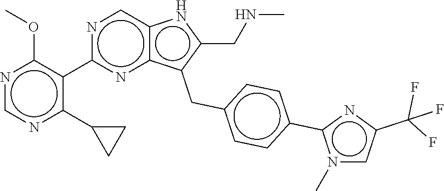 | 159 | | c | bb | ccc | B |
| 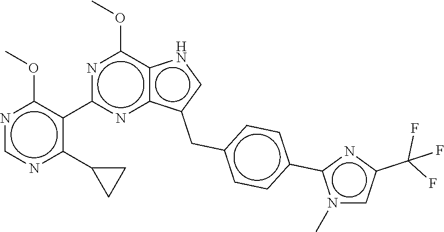 | 160 | | c | bb | ccc | C |
| 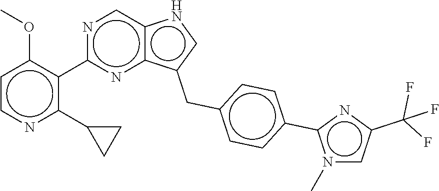 | 161 | | b | aa | ddd | B |
| 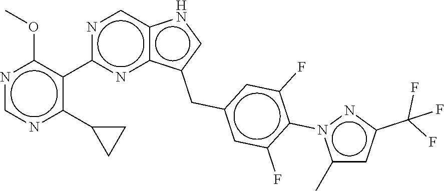 | 162 | | a | aa | ddd | A |
| 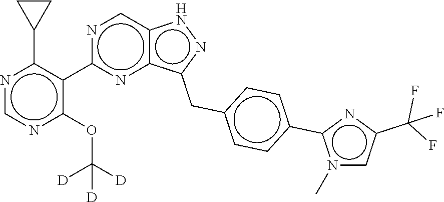 | 163 | | a | aa | ddd | A |
| 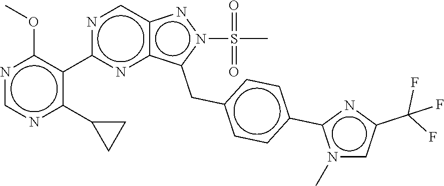 | 164 | | b | aa | ddd | A |
| 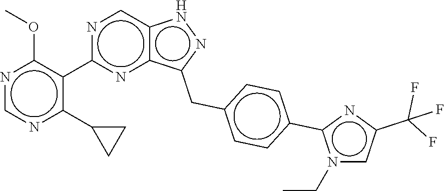 | 165 | | a | aa | ddd | A |
| 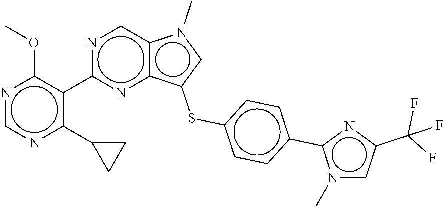 | 166 | | c | bb | ddd | C |
| 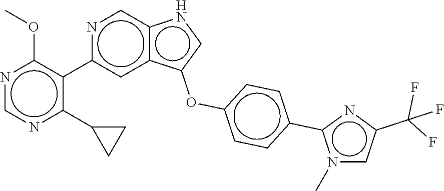 | 167 | | b | aa | ddd | A |
| 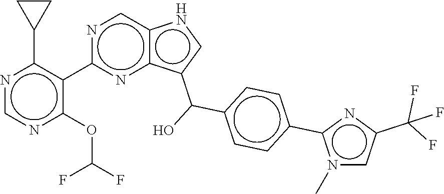 | 168 | | b | aa | ccc | A |
| 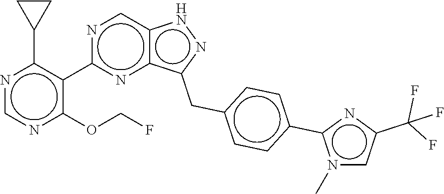 | 169 | | a | | | |
| 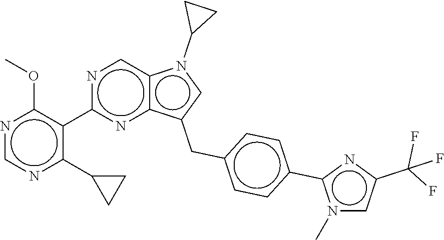 | 170 | | a | | | |
| 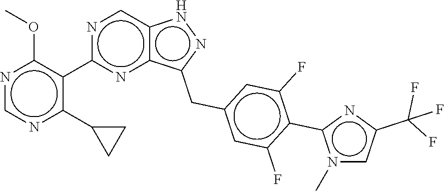 | 171 | | a | aa | ddd | A |
| 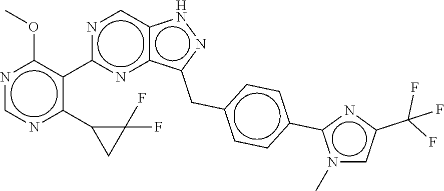 | 172 | | a | aa | ddd | A |
| 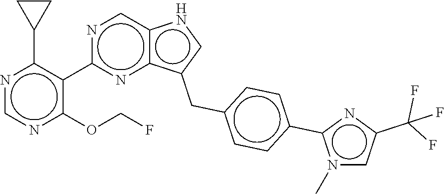 | 173 | | a | aa | ddd | |
| 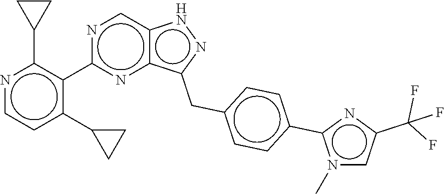 | 174 | | a | bb | ddd | B |
| 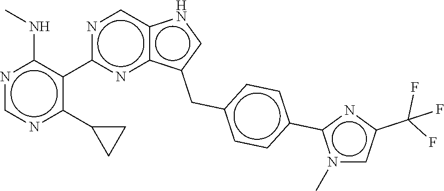 | 175 | | c | bb | ddd | B |
| 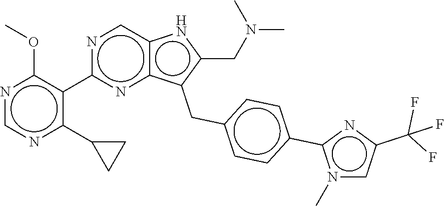 | 176 | | b | aa | ddd | A |
| 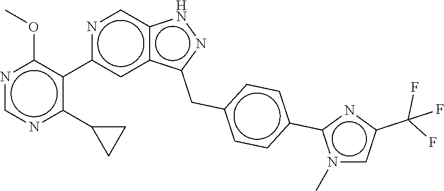 | 177 | | a | aa | ddd | A |
| 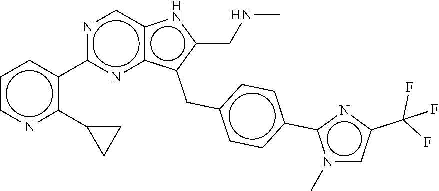 | 178 | | b | aa | ccc | A |
| 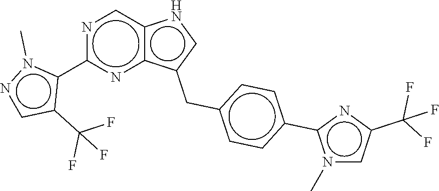 | 179 | | a | aa | ccc | A |
| 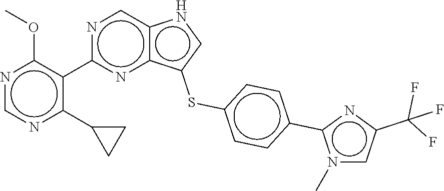 | 180 | | c | bb | ddd | B |
| 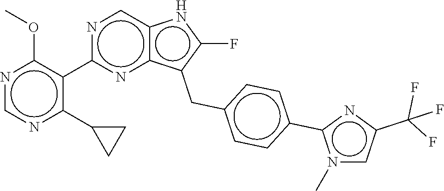 | 181 | | a | aa | ddd | A |
| 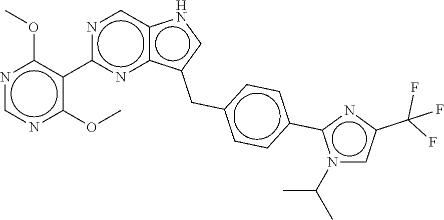 | 182 | | b | bb | ddd | A |
| 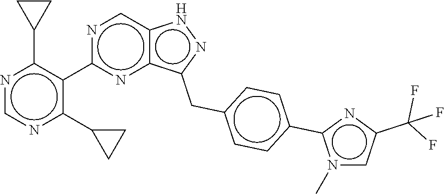 | 183 | | a | aa | ddd | A |
| 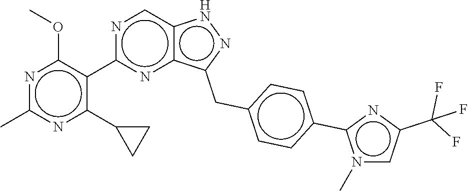 | 184 | | b | aa | ccc | A |
| 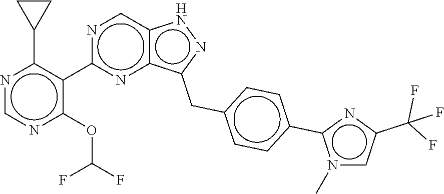 | 185 | | a | aa | ddd | A |
| 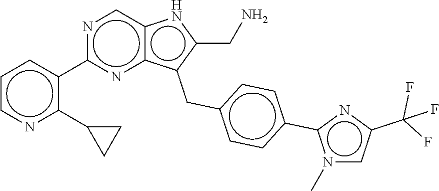 | 186 | | b | bb | ccc | B |
| 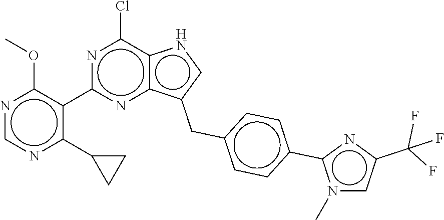 | 187 | | a | aa | ccc | |
| 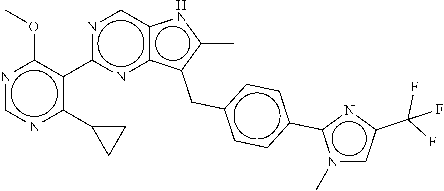 | 188 | | a | aa | ddd | A |
| 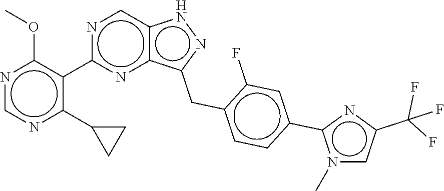 | 189 | | b | aa | ddd | A |
| 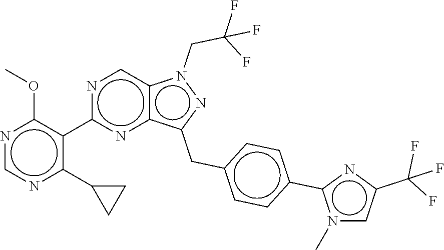 | 190 | | b | aa | ddd | A |
| 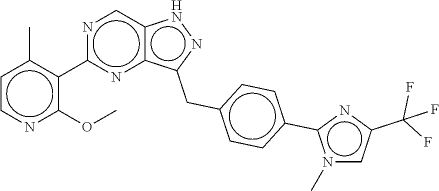 | 191 | | c | bb | ddd | C |
| 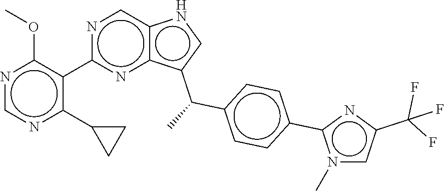 | 192 | or | a | aa | ccc | A |
| 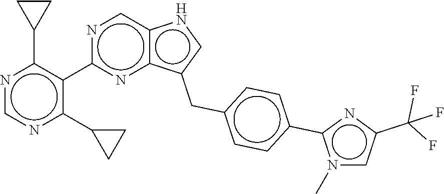 | 193 | | a | aa | ddd | A |
| 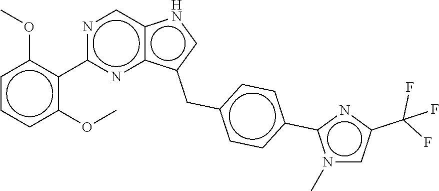 | 194 | | b | bb | ddd | C |
| 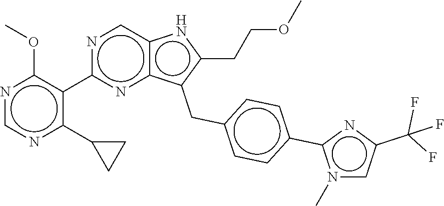 | 195 | | a | aa | ddd | A |
| 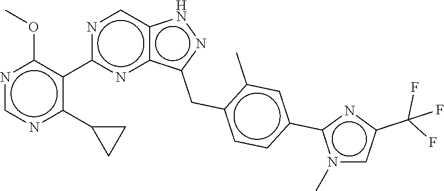 | 196 | | a | aa | ddd | A |
| 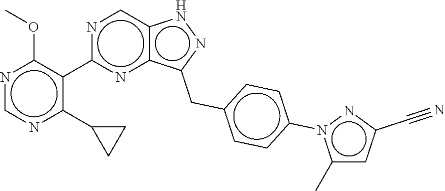 | 197 | | b | bb | ddd | C |
| 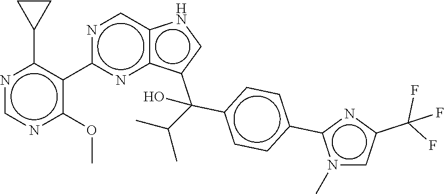 | 199 | | c | bb | ccc | B |
| 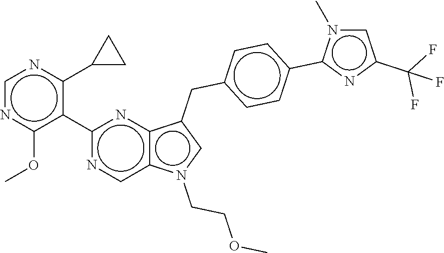 | 200 | | b | aa | ddd | A |
| 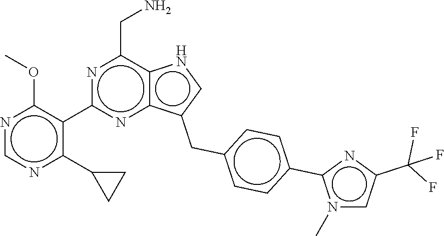 | 201 | | c | | | |
| 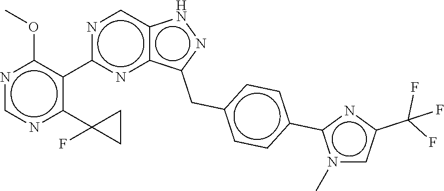 | 202 | | a | aa | ddd | A |
| 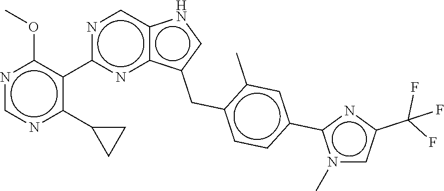 | 203 | | a | aa | ddd | A |
| 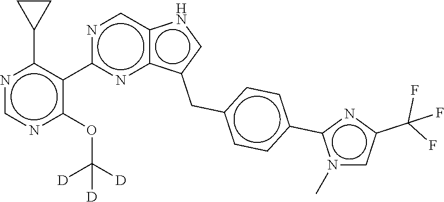 | 204 | | a | aa | ddd | A |
| 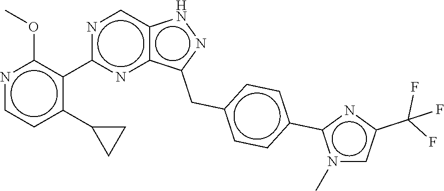 | 205 | | a | aa | ddd | A |
| 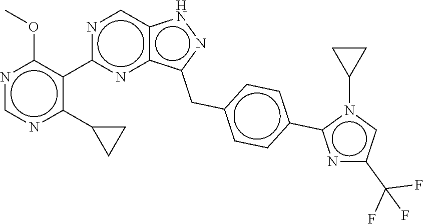 | 206 | | a | aa | ccc | A |
| 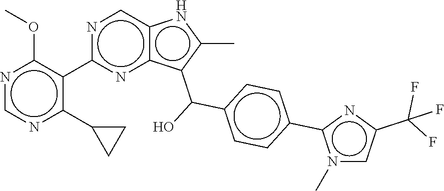 | 207 | | c | cc | ddd | C |
| 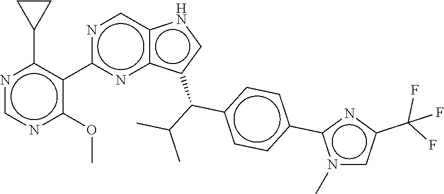 | 208 | or | c | cc | ccc | |

### Alternative Embodiments

In an alternative embodiment, compounds described herein may also comprise one or more isotopic substitutions, for example, hydrogen may be ²H (D or deuterium) ³H (T or tritium); carbon may be, for example, ¹³C or ¹⁴C; oxygen may be, for example, 80; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, provided is a pharmaceutical composition comprising a pharmaceutically acceptable carrier and an effective amount of a compound described herein (e.g., a compound of Formula (I), (II), (IIa), (IIa1), (IIb), (IIb1), (IIc), (IIc1), (IIc′), (IIc1′) or a compound of Table 1), or a pharmaceutically acceptable salt, hydrate, solvate, prodrug, stereoisomer, or tautomer thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-β-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In some embodiments, with respect to the pharmaceutical composition, the carrier is a parenteral carrier, oral or topical carrier.

In some embodiments, provided is a compound described herein (e.g., a compound of Formula (I), (II), (IIa), (IIa1), (IIb), (IIb1), (IIc), (IIc1), (IIc′), (IIc1′) or a compound of Table 1), or a pharmaceutically acceptable salt, hydrate, solvate, prodrug, stereoisomer, or tautomer thereof) (or pharmaceutical composition thereof) for use as a pharmaceutical or a medicament (e.g., a medicament for the treatment of a disease or disorder associated with USP1 in a subject in need thereof). In some embodiments, the disease is a proliferating disease. In a further embodiment, the disease is cancer. In some embodiments, the cancer is breast cancer (e.g., triple negative breast cancer), ovarian cancer (e.g., platinum-resistant ovarian cancer, platinum-refractory ovarian cancer), prostate cancer, pancreatic cancer or lung cancer (e.g., non-small cell lung cancer (NSCLC)).

In some embodiments, provided is a compound described herein (e.g., a compound of Formula (I), (II), (IIa), (IIa1), (IIb), (IIIb), (IIc), (IIc1), (IIc′), (IIc1′) or a compound of Table 1), or a pharmaceutically acceptable salt, hydrate, solvate, prodrug, stereoisomer, or tautomer thereof) (or pharmaceutical composition thereof) for use in the treatment of a disease or disorder associated with USP1 in a subject in need thereof. In some embodiments, the disease is a proliferating disease. In a further embodiment, the disease is cancer. In some embodiments, the cancer is breast cancer (e.g., triple negative breast cancer), ovarian cancer (e.g., platinum-resistant ovarian cancer, platinum-refractory ovarian cancer), prostate cancer, pancreatic cancer or lung cancer (e.g., non-small cell lung cancer (NSCLC)).

In some embodiments, provided is a compound described herein (e.g., a compound of Formula (I), (II), (IIa), (IIa1), (IIb), (IIb1), (IIc), (IIc1), (IIc′), (IIc1′) or a compound of Table 1), or a pharmaceutically acceptable salt, hydrate, solvate, prodrug, stereoisomer, or tautomer thereof) (or pharmaceutical composition thereof) for use in the manufacturing of a medicament (e.g., a medicament for the treatment of an a disease or disorder associated with USP1 in a subject in need thereof). In some embodiments, the disease is a proliferating disease. In a further embodiment, the disease is cancer. In some embodiments, the cancer is breast cancer (e.g., triple negative breast cancer), ovarian cancer (e.g., platinum-resistant ovarian cancer, platinum-refractory ovarian cancer), prostate cancer, pancreatic cancer or lung cancer (e.g., non-small cell lung cancer (NSCLC)). Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose.gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable non irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds disclosed herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided are pharmaceutically acceptable acid addition salt of a compound described herein (e.g., compound of Formula (I), (II), (IIa), (IIa1), (IIb), (IIb1), (IIc), (IIc1), (IIc′), (IIc1′) or a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Methods of Treatment and Use

In one embodiment, the compounds described herein can be used to inhibit the activity of a USP1 protein. For example, in some embodiments, a method of inhibiting a USP1 protein comprises contacting the USP1 protein with a compound disclosed herein. The contacting can occur in vitro or in vivo.

In some embodiments, the compounds described herein can be used to treat a “USP1 protein mediated” disorder (e.g., a USP1 protein mediated cancer), a “USP1 associated” disorder (e.g., a USP1 associated cancer), or a disorder “associated with USP1” (e.g., a cancer associated with USP1). A “USP1 protein mediated”, “USP1 associated” disorder or a disorder “associated with USP1”, is any pathological condition in which a USP1 protein is known to play a role, including any cancers that require USP1 for cell proliferation and survival. In some embodiments, “USP1 protein mediated”, “USP1 associated” disorder or a disorder “asssociated with USP1” is a proliferative disease such as cancer. The method comprises administering to a patient in need of a treatment for aUSP1 protein mediated disorder an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof or a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient.

In some embodiments, provided is a method of treating a disease or disorder associated with modulation of USP1. The method comprises administering to a patient in need of a treatment for diseases or disorders associated with modulation of ubiquitin specific protease 1 (USP1) an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof or a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient. In some embodiments the disease or disorder is cancer. In some embodiments, the compound or composition is administered in combination with a second therapeutic agent.

In some embodiments, provided is a method of treating or preventing cancer. The method comprises administering to a patient in need of a treatment for cancer an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof or a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient.

In some embodiments, provided is a method of treating cancer. The method comprises administering to a patient in need thereof of a treatment for cancer an effective amount of a compound of Formula (I), (II), (IIa), (IIa1), (IIb), (IIb1), (IIc), (IIc1), (IIc′), (IIc′) or a compound of Table 1 or a pharmaceutically acceptable salt, hydrate, solvate, prodrug, tautomer or stereoisomer thereof or a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient.

In some embodiments, provided is a method of treating or preventing a disease or disorder associated with DNA damage. The method comprises administering to a patient in need of a treatment for diseases or disorders associated with DNA damage an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof or a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient. In some embodiments the disease is cancer.

In some embodiments, provided is a method of treating a disease or disorder associated with DNA damage. The method comprises administering to a patient in need of a treatment for diseases or disorders associated with DNA damage an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof or a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient.

In some embodiments, provided is a method of inhibiting, modulating or reducing DNA repair activity exercised by USP1. The method comprises administering to a patient in need thereof an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof or a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient.

In some embodiments, provided is (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient, for use as a medicament.

In some embodiments, provided is (a) a compound of Formula (I), (II), (IIa), (IIa1), (IIb), (IIb1), (IIc), (IIc1), (IIc′), (IIc′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient for use in the treatment or prevention of a disease associated with inhibiting USP1. In some embodiments the disease is cancer.

In some embodiments, provided is (a) a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof, or (b) a pharmaceutical composition comprising an effective amount of a compound of Formula (I), (II), (IIa), (IIa1), (IIb), (IIb1), (IIc1), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient for use in the treatment of a disease or disorder associated with inhibiting USP1.

In some embodiments, provided is (a) a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient for use in the treatment or prevention of cancer.

In some embodiments, provided is (a) a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, tautomer or stereoisomer thereof and a pharmaceutically acceptable excipient for use in the treatment of cancer.

In some embodiments, provided is (a) a compound of Formula (I), (II), (IIa), (IIa1), (IIb), (IIb1), (IIc), (IIc1), (IIc′), (Ic′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient for use in the treatment or prevention of a disease or disorder associated with DNA damage. In some embodiments the disease or disorder is cancer.

In some embodiments, provided is (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient for use in the treatment of a disease or disorder associated with DNA damage. In some embodiments the disease or disorder is cancer.

In some embodiments, provided is (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient for use in a method of inhibiting or reducing DNA repair activity modulated by USP1.

In some embodiments, provided is a compound of Formula (I), (II), (IIa), (IIa1), (IIb), (IIb1), (IIc), (IIc1), (IIc′), (II1c′) or a compound of Table 1, or a pharmaceutical composition comprising a compound of Formula (I), (II), (IIa), (IIa1), (IIb), (IIb1), (IIc), (IIc1), (IIc′), (IIc1′) or a compound of Table 1 and a pharmaceutically acceptable carrier used for the treatment of cancers.

In some embodiments, provided is the use of (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient in the manufacture of a medicament for treating or preventing a disease associated with inhibiting USP1. In some embodiments the disease or disorder is cancer.

In some embodiments, provided is the use of (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient in the manufacture of a medicament for treating or preventing cancer.

In some embodiments, provided is the use of (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient in the manufacture of a medicament for treating or preventing a disease or disorder associated with DNA damage. In some embodiments, the disease or disorder is cancer.

In some embodiments, provided is the use of (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient in the manufacture of a medicament for treating a disease or disorder associated with DNA damage. In some embodiments, the disease or disorder is cancer.

In some embodiments, provided is the use of (a) a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient in the manufacture of a medicament for inhibiting or reducing DNA repair activity modulated by USP1.

In some embodiments, provided is a pharmaceutical composition comprising a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

In some embodiments, provided are methods of treating a disease or disorder associated with modulation of USP1 including, but not limited to, cancer comprising, administering to a patient suffering from at least one of said diseases or disorder (a) an effective amount (e.g., a therapeutically effective amount) of a compound of Formula (I), (II), (IIa), (IIa1), (IIb), (IIb1), (IIc), (IIc1), (Ic′), (IIc1′) or a compound of Table 1, or a pharmaceutically acceptable salt, hydrate, solvate, prodrug, stereoisomer, or tautomer thereof or (b) a pharmaceutical composition comprising an effective amount (e.g., a therapeutically effective amount) of a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof and a pharmaceutically acceptable excipient; and one or more additional anti-cancer agent(s).

In some embodiments, the compound disclosed herein (e.g., a compound of Formula (I), (II), (IIa), (IIa1), (IIb), (IIb1), (IIc), (IIc1), (IIc′), (IIc1′) or a compound of Table 1, or a pharmaceutically acceptable salt, hydrate, solvate, prodrug, stereoisomer, or tautomer thereof) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound disclosed herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In some embodiments, provided are kits that include one or more of the compounds disclosed herein (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) and a second therapeutic agent as disclosed herein are provided. Representative kits include (a) a compound disclosed herein or a pharmaceutically acceptable salt, hydrate, solvate, prodrug, stereoisomer, or tautomer thereof (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), (b) at least one other therapeutic agent, e.g., as indicated above, whereby such kit may comprise a package insert or other labeling including directions for administration.

In some embodiments of the methods and uses described herein, the cancer is selected from adrenocortical carcinoma, AIDS-related lymphoma, AIDS-related malignancies, anal cancer, cerebellar astrocytoma, extrahepatic bile duct cancer, bladder cancer, osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cance, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g., renal pelvis and ureter), retinoblastoma, rhabdomyosarcoma, salivary gland cancer, malignant fibrous histiocytoma of bone, soft tissue sarcoma, sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor. In other embodiments, the cancer is a non-small cell lung cancer.

In any of the embodiments, the cancer can be any cancer in any organ, for example, a cancer is selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

In some embodiments, the cancer to be treated with a compound disclosed herein is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

In other embodiments, the cancer is selected from liposarcoma, neuroblastoma, glioblastoma, bladder cancer, adrenocortical cancer, multiple myeloma, colorectal cancer, non-small cell lung cancer, Human Papilloma Virus-associated cervical, oropharyngeal, penis, anal, thyroid or vaginal cancer or Epstein-Barr Virus-associated nasopharyngeal carcinoma, gastric cancer, rectal cancer, thyroid cancer, Hodgkin lymphoma and diffuse large B-cell lymphoma.

In some embodiments, the cancer is selected from breast cancer (e.g., triple negative breast cancer (TNBC)), ovarian cancer (e.g., platinum-resistant ovarian cancer, platinum-refractory ovarian cancer), prostate cancer, pancreatic cancer and lung cancer (e.g., non-small cell lung cancer (NSCLC)). In some embodiments, the cancer is selected from breast cancer (e.g., triple negative breast cancer (TNBC)), ovarian cancer (e.g., platinum-resistant ovarian cancer, platinum-refractory ovarian cancer), prostate cancer and lung cancer (e.g., non-small cell lung cancer (NSCLC)). In some embodiments, the cancer is breast cancer. In some embodiments the cancer is triple negative breast cancer (TNBC). In some embodiments the cancer is prostate cancer. In some embodiments the cancer is lung cancer. In some embodiments the cancer is non-small cell lung cancer (NSCLC).

In certain embodiments of the methods described herein, the cancer is a dedifferentiated ID-driven cancer. In other embodiments, the cancer is a cancer that is sensitive to USP1 inhibition. In yet other embodiments, the cancer is a cancer that is sensitive to USP1 inhibition due to DNA damage pathway deficiency.

In some embodiments of the methods and uses described herein, the cancer is selected from the group consisting of a hematological cancer, a lymphatic cancer, and a DNA damage repair pathway deficient cancer.

In some embodiments, a compound disclosed herein is used to treat a cancer, wherein the cancer is a homologous recombination deficient cancer. In some embodiments, a compound disclosed herein is used to treat a cancer that does not have a defect in the homologous recombination pathway.

In some embodiments, the cancer is a DNA damage repair pathway deficient cancer. In some embodiments, the DNA damage repair pathway deficient cancer is selected from the group consisting of lung cancer, non-small cell lung cancer (NSCLC), colon cancer, bladder cancer, osteosarcoma, ovarian cancer (e.g., platinum-resistant ovarian cancer, platinum-refractory ovarian cancer), and breast cancer (e.g., triple negative breast cancer (TNBC). In some embodiments, the cancer is non-small cell lung cancer (NSCLC). In some embodiments, the cancer is colon cancer. In some embodiments, the cancer is bladder cancer. In some embodiments, the cancer is ovarian cancer or breast cancer. In some embodiments, the cancer is ovarian cancer. In some embodiments, the cancer is platinum-resistant ovarian cancer. In some embodiments, the cancer is platinum-refractory ovarian cancer. In some embodiments, the cancer is breast cancer. In further embodiments, the cancer is triple negative breast cancer.

In some embodiments, the cancer is a HRR (homologous recombination repair) gene mutant cancer. In some embodiments, the cancer is a HRR (homologous recombination repair) gene mutant cancer selected from the group consisting of ATM, BARD1, BRCA1, BRCA2, BRIP1, CDK12, CHEK1, CHEK2, FANCL, PALB2, PPP2R2A, RAD51B, RAD51C, RAD51D, or RAD54L mutant cancer. In some embodiments, the cancer is an ATM mutant cancer. In some embodiments, the cancer is an BARD1 mutant cancer. In some embodiments, the cancer is an BRCA1 mutant cancer. In some embodiments, the cancer is an BRCA2 mutant cancer. In some embodiments, the cancer is an BR1P1 mutant cancer. In some embodiments, the cancer is an CDK12 mutant cancer. In some embodiments, the cancer is an CHEK1 mutant cancer. In some embodiments, the cancer is an CHEK2 mutant cancer. In some embodiments, the cancer is an FANCL mutant cancer. In some embodiments, the cancer is an PALB2 mutant cancer. In some embodiments, the cancer is an PPP2R2A mutant cancer. In some embodiments, the cancer is an RAD51B mutant cancer. In some embodiments, the cancer is an RAD51C mutant cancer. In some embodiments, the cancer is an RAD51D mutant cancer. In some embodiments, the cancer is an RAD54L mutant cancer

In some embodiments, the cancer is a BRCA1 mutant cancer. In some embodiments, the BRCA1 mutation is a germline mutation. In some embodiments, the BRCA1 mutation is a somatic mutation. In some embodiments, the BRCA1 mutation leads to BRCA1 deficiency. In some embodiments, the cancer is a BRCA2 mutant cancer. In some embodiments, the BRCA2 mutation is a germline mutation. In some embodiments, the BRCA2 mutation is a somatic mutation. In some embodiments, the BRCA2 mutation leads to BRCA2 deficiency. In some embodiments, the cancer is a BRCA1 mutant cancer and a BRCA2 mutant cancer. In some embodiments, the cancer is a BRCA1 deficient cancer. In some embodiments, the cancer is a BRCA2 deficient cancer. In some embodiments, the cancer is a BRCA1 deficient cancer and a BRCA2 deficient cancer. In some embodiments, the cancer is not a BRCA1 mutant cancer or a BRCA2 mutant cancer. In some embodiments, the cancer is a BRCA1 deficient cancer and a BRCA2 mutant cancer. In some embodiments, the BRCA1 or BRCA2 mutant or BRCA1 or BRCA2 deficient cancer is selected from non-small cell lung cancer (NSCLC), osteosarcoma, prostate cancer, pancreatic cancer, ovarian cancer, and breast cancer. In some embodiments, the BRCA1 mutant, BRCA2 mutant, BRCA1 deficient or BRCA 2 deficient cancer as described herein is ovarian cancer, breast cancer, prostate cancer or pancreatic cancer. In some embodiments, the cancer is ovarian cancer. In some embodiments, the cancer is platinum-resistant ovarian cancer. In some embodiments, the cancer is platinum-refractory ovarian cancer. In some embodiments, the cancer is breast cancer. In some embodiments, the cancer is a triple negative breast cancer. In some embodiments, the cancer is prostate cancer. In some embodiments, the cancer is homologous recombination deficient. Homologous recombination deficiency can be measured by BRCA1/2 mutation, or genomic instability (positive homologous recombination deficiency (HRD) score) without BRCA1/2 mutations.

In some embodiments, the cancer is a Poly (ADP-ribose) polymerase (“PARP”) inhibitor refractory or resistant cancer. In some embodiments, the cancer is a PARP inhibitor resistant or refractory BRCA1, BRCA2, or BRCA1 and BRCA2 mutant cancer. In some embodiments, the cancer is a PARP inhibitor resistant or refractory BRCA1, BRCA2, or BRCA1 and BRCA2-deficient cancer. In some embodiments, the PARP inhibitor refractory or resistant cancer is selected from the cancers described herein. In some embodiments, the PARP inhibitor refractory or resistant cancer is selected from breast cancer (e.g., triple negative breast cancer (TNBC), ovarian cancer (e.g., platinum-resistant ovarian cancer, platinum-refractory ovarian cancer), pancreatic cancer and prostate cancer).

In some embodiments, the cancer has a mutation in the gene encoding ataxia telangiectasia mutated (ATM) protein kinase or loss of ATM protein expression. In some embodiments, the cancer to be treated with a compound disclosed herein is a cancer (e.g., a cancer selected from the cancers described herein) that comprises cancer cells with a loss of function mutation in a gene encoding ATM. In some embodiments the ATM mutation is a germline mutation. In some embodiments the ATM mutation is a somatic mutation. In some embodiments, the cancer is not an ATM mutant cancer. In some embodiments the cancer is an ATM-deficient cancer. In some embodiments, the ATM-deficient cancer is selected from breast cancer (e.g., triple negative breast cancer (TNBC)), ovarian cancer (e.g., platinum-resistant ovarian cancer, platinum-refractory ovarian cancer), colorectal cancer, stomach cancer, endometrial cancer, urothelial cancer, cervical cancer, melanoma, esophageal cancer, head and neck cancer, mantle cell lymphoma, sarcoma, prostate cancer, pancreatic cancer, and lung cancer (e.g., non-small cell lung cancer (NSCLC)).

In some embodiments, the cancer comprises cancer cells with elevated levels of translesion synthesis. This includes cancers that exhibit elevated PCNA monoubiquitination, with or without elevated levels of RAD18 and/or UBE2K. In some embodiments, the elevated levels of RAD18 and/or UBE2K are elevated RAD18 and/or UBE2K protein levels. In some embodiments, the elevated levels of RAD18 and/or UBE2K are elevated RAD18 and/or UBE2K mRNA levels. In some embodiments, elevated levels of RAD18 and/or UBE2K (e.g., RAD18 and/or UBE2K protein and/or RAD18 and/or UBE2K mRNA) have been detected (e.g., in a cancer sample obtained from the subject) prior to the administration. Elevated translesion synthesis can also be measured by PCNA monoubiquitination without elevated RAD18 and/or UBE2K levels. In some embodiments, a subject's cancer has been tested for RAD18 and/or UBE2K levels protein or mRNA, or PCNA monoubiquitination prior to beginning treatment with a USP1 inhibitor. In some embodiments, the cancer is a breast cancer (e.g., triple negative breast cancer), an ovarian cancer, a lung cancer (e.g., non-small cell lung cancer (NSCLC)), or a prostate cancer.

In some embodiments, the cancer is a BRCA1 and/or BRCA2 mutant cancer, wherein the cancer comprises cells with increased translesion synthesis, as exemplified by elevated PCNA monoubiquitination with or without elevated RAD18 and/or UBE2K levels. In some embodiments, the cancer is a breast cancer (e.g., triple negative breast cancer), an ovarian cancer or a prostate cancer that is a BRCA1 and/or BRCA2 mutant cancer.

In some embodiments, the cancer is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

### Combination Therapies

In some embodiments, the compounds of the disclosure are administered in therapeutically effective amounts in a combination therapy with one or more therapeutic agents (pharmaceutical combinations) or modalities, e.g. non-drug therapies. For example, synergistic effects can occur with other anti-proliferative, anti-cancer, immunomodulatory or anti-inflammatory substances. Where the compounds of the disclosure are administered in conjunction with other therapies, dosages of the co-administered compounds will vary depending on the type of co-drug employed, on the specific drug employed, on the condition being treated and so forth.

In some embodiments, provided are methods of treatment of a disease or disorder associated with the USP1 with a compound of Formula (I), (II), (IIa), (IIa1), (IIb), (IIb1), (IIc). (IIc1), (IIc′), (IIc1′) or a compound of Table 1 or a pharmaceutically acceptable salt, hydrate, solvate, prodrug, stereoisomer or tautomer thereof) in combination with a second therapeutic agent. In some embodiments, provided are methods of treatment of a disease or disorder associated with USP1 with a compound of Formula (I), (II), (IIa), (IIa1), (IIb), (IIb1), (IIc), (IIc1), (IIc′), (IIc1′) or a compound of Table 1 or pharmaceutically acceptable salts thereof in combination with a second therapeutic agent and a third therapeutic agent. In some embodiments, provided are methods of treatment of a disease or disorder associated with the USP1 with a compound of Formula (I), (II), (IIa), (IIa1), (IIb), (IIb1), (IIc), (IIc1), (IIc′), (IIc1′) or a compound of Table 1, or pharmaceutically acceptable salts thereof) in combination with a second therapeutic agent, a third therapeutic agent, and a fourth therapeutic agent.

The term “Combination” refers to either a fixed combination in one dosage unit form, or a combined administration where a compound disclosed herein (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound disclosed herein (e.g., a compound of Formula (I), (II), (IIa), (IIa1), (IIb), (IIb1), (IIc), (IIc1), (IIc′), (IIc′) or a compound of Table 1 or a pharmaceutically acceptable salt, hydrate, solvate, prodrug, stereoisomer or tautomer thereof) and a combination partner, are both administered to a patient simultaneously in the form of a single entity or dosage. The term “non-fixed combination” means that the therapeutic agents, e.g., a compound disclosed herein (e.g., a compound of Formula (I), (II), (IIa), (IIa1), (IIb), (IIb1), (IIc), (i d), (IIc′), (IIc1′) or a compound of Table 1 or a pharmaceutically acceptable salt, hydrate, solvate, prodrug, stereoisomer or tautomer thereof) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients.

Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, compounds disclosed herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and a general chemotherapeutic agent selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan, or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, RubexX), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEXt), irinotecan (Camptosar®), L-asparaginase (ELSPARk), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadext), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1). (IIb), (IIb1), (IIc), (IIc1), (IIc′), (IIc1′) or a compound of Table 1 or pharmaceutically acceptable salts thereof) and an EGFR-inhibitor (e.g., cetuximab, panitumimab, erlotinib, gefitinib and EGFRi NOS). In some embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and a MAPK-pathway inhibitor (e.g., BRAFi, panRAFi, MEKi, ERKi) in some embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and a PI3K-mTOR pathway inhibitor (e.g., alpha-specific PI3Ki, pan-class I PI3Ki and mTOR/PI3Ki, particularly everolimus and analogues thereof).

In some embodiments, provided is a method of enhancing the chemotherapeutic treatment of cancer in a mammal undergoing treatment with an anti-cancer agent, which method comprises co-administering to the mammal an effective amount of a compound disclosed herein. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and a DNA damaging agent (e.g., actinomycin, amsacrine, anthracyclines, bleomycin, busulfan, camptothecin, carboplatin, chlorambucil, cisplatin, cyclophosphamide.

Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, tenyposide, triethylenethiophosphoramide and etoposide). In a preferred embodiment, the DNA damaging agent is cisplatin. In some embodiments, the DNA damaging agent is radiation or a biotherapeutic agent (e.g., an antibody).

In some embodiments, the anti-cancer agent is selected from reversible DNA binders (e.g., topotecan hydrochloride, irinotecan (CPT11—Camptosar), rubitecan, exatecan, nalidixic acid, TAS-103, etoposide, acridines (e.g., amsacrine, aminocrine), actinomycins (e.g., actinomycin D), anthracyclines (e.g., doxorubicin, daunorubicin), benzophenainse, XR 1 1576/MLN 576, benzopyridoindoles, Mitoxantrone, AQ4, Etoposide, Teniposide, epipodophyllotoxins, and bisintercalating agents such as triostin A and echinomycin), DNA alkylators (e.g., sulfur mustard, the nitrogen mustards (e.g., mechlorethamine), chlorambucil, melphalan, ethyleneimines (e.g., triethylenemelamine, carboquone, diaziquone), methyl methanesulfonate, busulfan, CC-1065, duocarmycins (e.g., duocarmycin A, duocarmycin SA), metabolically activated alkylating agents such as nitrosoureas (e.g., carmustine, lomustine, (2-chloroethyl)nitrosoureas), triazine antitumor drugs such as triazenoimidazole (e.g., dacarbazine), mitomycin C and leinamycin), DNA strand breakers (e.g., doxorubicin and daunorubicin (which are also reversible DNA binders), other anthracyclines, bleomycins, tirapazamine, enediyne antitumor antibiotics such as neocarzinostatin, esperamicins, calicheamicins, dynemicin A, hedarcidin, C-1027, N1999A2, esperamicins and zinostatin), and disruptors of DNA replication (e.g., 5-fluorodeoxyuridine).

In certain embodiments, the DNA damaging agent is radiation (e.g., radiation that induces a DNA cross-linking in a cell when applied to the cell, (e.g., ionizing radiation and ultraviolet (UV) radiation)). Ionizing radiation consists of subatomic particles or electromagnetic waves that are sufficiently energetic to cause ionization by detaching electrons from atoms or molecules. Ionization depends on the energy of the impinging individual particles or waves. In general, ionizing particles or photons with energies above a few electron volts can be ionizing. Non-limiting examples of ionizing particles are alpha particles, beta particles, and neutrons. The ability of photons to ionize a atom or molecule depends on its frequency. Short-wavelength radiation such as high frequency ultraviolet, x-rays, and gamma rays, is ionizing. Ionizing radiation comes from radioactive materials, x-ray tubes, and particle accelerators.

In certain embodiments, the anticancer agent targets a USP1 independent mechanism of DNA repair. Non-limiting examples of suitable DNA repair inhibitors are poly (ADP-ribose) polymerase (PARP) inhibitors, DNA-dependent protein kinase (DNA-PK) inhibitors, ataxia telangiectasia and Rad3-related protein (ATR) inhibitors, ataxia-telangiectasia mutated (ATM) inhibitors, checkpoint kinase 1 (CHK1) inhibitors, checkpoint kinase 2 (CHK2) inhibitors, and Wee1 inhibitors. It has been reported that BRCA1/2 status predicts the efficacy of PARP inhibitors in the clinic (Audeh et al. Lancet (2010) 376 (9737), 245-51). In general, BRCA1/2 mutant cancers have increased sensitivity to USP1 inhibitors. Accordingly, in some embodiments, a In certain embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and a PARP inhibitor (e.g., olaparib, rucaparib, niraparib, talazoparib, and veliparib).

In certain embodiments, the anticancer or DNA damaging agent can be a biotherapeutic. Non-limiting examples of suitable biotherapeutics include rInterferon-a₂a, rInterferon-oi2b, rInterteukin-2, rG-CSF, rGM-CSF, and rErythropoietin.

In certain embodiments, the anticancer agent can be an antibody, such as a monoclonal antibody. Non-limiting examples of suitable therapeutic monoclonal antibodies for use in the methods described herein include trastuzumab, an anti-ErbB2/HER2 for breast cancer, cetuximab, an anti-ErbBl/EGFR for colorectal cancer, and bevacizumab, an anti-VEGF for colorectal, breast and lung cancers (G. Adams et al., Nature Biotechnology 23:1 147-57 (2005)). Multitarget inhibitors, such as Sutent which inhibits TK activity of VEGFR, PDGFR and FGFR, are also suitable for use in the inventive method.

In certain embodiments, the anticancer agent can be a proteasome inhibitor, such as bortezomib.

Administration of the compounds disclosed herein can be accomplished via any mode of administration of therapeutic agents including systemic or local administration such as oral, nasal, parenteral, transdermal, subcutaneous, vaginal, buccal, rectal or topical administration modes.

Some patients may experience allergic reactions to the compounds disclosed herein and/or other anti-cancer agent(s) during or after administration; therefore, anti-allergic agents are often administered to minimize the risk of an allergic reaction. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and an anti-allergic agent (e.g., corticosteroids, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®-, hydrocortisone phosphate, Solu-Cortef®, Hydrocort Acetate® and Lanacort®), prednisolone (sold under the tradenames Delta-Cortel®, Orapred®, Pediapred®, and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol®, and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®)).

Some patients may experience nausea during and after administration of the compound disclosed herein and/or other anti-cancer agent(s); therefore, anti-emetics are used in preventing nausea (upper stomach) and vomiting. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and an anti-emetic (e.g., aprepitant (Emend®), ondansetron (Zofran®), granisetron HCl (Kytril®), lorazepam (AtivanA, dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIb1), (IIc), (IIc1), (IIc′), (IIc1′) or a compound of Table 1 or pharmaceutically acceptable salts thereof) and an analgesic (e.g., an over-the-counter analgesics, (e.g., Tylenol®), an opioid analgesic (e.g., hydrocodone/paracetamol or hydrocodone/acetaminophen (e.g., Vicodin®), morphine (e.g., Astramorph® or Avinza®), oxycodone (e.g., OxyContint or PercocetV), oxymorphone hydrochloride (Opana®, and fentanyl (e.g., Duragesic®)).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II), (IIa), (IIa1), (IIb), (IIIb1), (IIc), (IIc1), (IIIc′), (IIc1′) or a compound of Table 1 or pharmaceutically acceptable salts thereof) and a cytoprotective agent (e.g., Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a compound disclosed herein, can be prepared and administered as described in the art, including, but not limited to, in the documents cited above.

In some embodiments, provided are pharmaceutical compositions comprising at least one compound disclosed herein (e.g., a USP1 inhibitor, e.g., a compound of Formula (I), (II), (IIa), (IIa1), (IIb), (II1), (IIc), (IIc1), (IIc′), (IIc1′) or a compound of Table 1 or a pharmaceutically acceptable salt, hydrate, solvate, prodrug, stereoisomer or tautomer thereof together with a pharmaceutically acceptable carrier suitable for administration to a human or animal subject, either alone or together with other anti-cancer agents.

In some embodiments, provided are methods of treating human or animal subjects having or having been diagnosed with a disease or disorder associated with USP1 (e.g., cancer) comprising administering to the subject in need thereof a therapeutically effective amount of a compound disclosed herein (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof in combination with a second therapeutic agent.

In some embodiments, provided are methods of treating a a disease or disorder associated with USP1 (e.g., cancer) in a subject in need thereof comprising administering to the subject a therapeutically effective amount of a compound disclosed herein (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof in combination with a second therapeutic agent.

In particular, compositions will either be formulated together as a combination herapeutic or administered separately.

In combination therapy, the compound disclosed herein and other anti-cancer agent(s) may be administered either simultaneously, concurrently or sequentially with no specific time limits, wherein such administration provides therapeutically effective levels of the two compounds in the body of the patient.

A compound disclosed herein (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) may also be used in combination with known therapeutic processes, for example, the administration of hormones or especially radiation. A compound disclosed herein may in particular be used as a radiosensitizer, especially for the treatment of tumors which exhibit poor sensitivity to radiotherapy.

In certain instances, compounds disclosed herein are combined with other therapeutic agents, including, but not limited to, other anti-cancer agents, anti-allergic agents, anti-nausea agents (or anti-emetics), pain relievers, cytoprotective agents, and combinations thereof.

### Patient Selection and Monitoring

Determining Whether a Subject Will Respond to Treatment with USP1 Inhibitors

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting levels of RAD18 and/or UBE2K (e.g., RAD18 and/or UBE2K protein and/or RAD18 and/or UBE2K mRNA) in a test cancer sample (e.g., in a cancer test sample obtained from the subject);
b) comparing the test cancer sample with reference cells (e.g., a reference sample taken from a non-cancerous or normal control subject), wherein elevated levels of RAD18 and/or UBE2K in said test cancer sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting levels of translesion synthesis (e.g., detecting PCNA monoubiquitination levels) in a test cancer sample (e.g., in a cancer test sample obtained from the subject);
b) comparing the test cancer sample with reference cells (e.g., a reference sample taken from a non-cancerous or normal control subject), wherein elevated translesion synthesis (e.g., increased PCNA monoubiquitination levels) in said test cancer sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting mutations in a gene encoding ATM (i.e, loss function mutations) in a test cancer sample (e.g., in a cancer test sample obtained from the subject);
b) wherein presence of mutations in a gene encoding ATM in said test cancer sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA1 (e.g., a loss of function mutation) in a subject test sample (e.g., in a cancer test sample or blood test sample obtained from the subject);
b) wherein presence of mutations in a gene encoding BRCA1 in said test sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer patient) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA2 (e.g., a loss of function mutation) in a subject test sample (e.g., in a cancer test sample or blood test sample obtained from the subject);
b) wherein presence of mutations in a gene encoding BRCA2 in said test sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc′) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (i), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting deficiency in homologous recombination (e.g., as measured by a positive homologous recombination deficiency (HRD) score) in a subject test sample (e.g., in a cancer sample or blood sample obtained from the subject)
b) wherein presence of homologous recombination deficiency (e.g., a positive homologous recombination deficiency (HRD) score) in said test sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

Determining if a Cancer Will Respond to Treatment with a USP1 Inhibitor

In some embodiments, provided is a method of determining if a cancer (e.g., a cancer associated with USP1) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting levels of RAD18 and/or UBE2K (e.g., RAD18 and/or UBE2K protein and/or RAD18 and/or UBE2K mRNA) a cancer test sample (e.g., in a cancer sample obtained from the subject)
b) comparing the cancer test sample with a reference (e.g., a reference sample taken from a non-cancerous or normal control subject), wherein elevated levels of RAD18 and/or UBE2K in said test sample indicates that the cancer will respond to therapeutic treatment with a USP1 inhibitor (e.g., a compound of Formula (I), (II), (IIa), (IIa1), (IIb), (II), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting levels of translesion synthesis (e.g., detecting PCNA monoubiquitination levels) in a test cancer sample (e.g., in a cancer sample obtained from the subject)
b) comparing the test cancer sample with a reference (e.g., a reference sample taken from a non-cancerous or normal control subject), wherein elevated translesion synthesis (e.g., increased PCNA monoubiquitination levels) in said test cancer sample indicates that the cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting mutations in a gene encoding ATM (i.e, loss function mutations) in a test cancer sample (e.g., in a cancer sample obtained from the subject)
b) wherein presence of mutations in a gene encoding ATM in said cancer sample indicates that the cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA1 (e.g., a loss of function mutation) in a cancer subject test sample (e.g., in a cancer sample or blood sample obtained from the cancer subject)
b) wherein presence of mutations in a gene encoding BRCA1 in said test sample indicates that the subject's cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA2 (e.g., a loss of function mutation) in a cancer subject test sample (e.g., in a cancer sample or blood sample obtained from the cancer subject)
b) wherein presence of mutations in a gene encoding BRCA2 in said test sample indicates that the subject's cancer will respond to therapeutic treatment with a USP1 inhibitor (e.g., a compound of Formula (I), (II), (IIa), (IIa1), (IIb), (IIb1), (IIc), (IIc1), (Ic′), (IIc1′) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting deficiency in homologous recombination (e.g., as measured by a positive homologous recombination deficiency (HRD) score) in a cancer subject test sample (e.g., in a cancer sample or blood sample obtained from the cancer subject)
b) wherein presence of homologous recombination deficiency in said test sample indicates that the subject's cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

### Determining Sensitivity of a Cancer Cell to USP1 Inhibition

In some embodiments, provided is a method of determining the sensitivity of a cancer cell to USP1 inhibition (e.g., inhibition with a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting levels of RAD18 and/or UBE2K (e.g., RAD18 and/or UBE2K protein and/or RAD18 and/or UBE2K mRNA) in a cancer cell test sample (e.g., in a cancer sample obtained from the subject)
b) comparing the test sample with a reference (e.g., a reference sample taken from a non-cancerous or normal control subject), wherein elevated levels of RAD18 and/or UBE2K in said test sample indicates said cancer cell is sensitive to USP1 inhibition.

In some embodiments, provided is a method of determining the sensitivity of a cancer cell to USP1 inhibition (e.g., inhibition with a compound of Formula (I), (II), (IIa), (IIa1), (IIb), (IIb1), (IIc), (IIIc1), (Ic′), (IIc1′) or a compound of Table 1 or a pharmaceutically acceptable salt, hydrate, solvate, prodrug, stereoisomer or tautomer thereof), comprising the steps of:
a) detecting levels of translesion synthesis (e.g., detecting PCNA monoubiquitination levels) in a cancer cell test sample (e.g., in a cancer sample obtained from the subject)
b) comparing the test sample with a reference (e.g., a reference sample taken from a non-cancerous or normal control subject), wherein elevated translesion synthesis (e.g., increased PCNA monoubiquitination levels) in said test sample indicates said cancer cell is sensitive to USP1 inhibition.

In some embodiments, provided is a method of determining the sensitivity of a cancer cell to USP1 inhibition (e.g., inhibition with a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting mutations in a gene encoding ATM (i.e, loss function mutations) in a cancer cell test sample (e.g., in a cancer sample obtained from a subject)
b) wherein presence of mutations in a gene encoding ATM in said cancer cell test sample indicates said cancer cell is sensitive to USP1 inhibition.

In some embodiments, provided is a method of determining the sensitivity of a cancer cell to USP1 inhibition (e.g., inhibition with a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting a mutation in a gene encoding BRCA1 (e.g., a loss of function mutation) in a cancer cell test sample (e.g., in a cancer sample obtained from a subject)
b) wherein presence of mutations in a gene encoding BRCA1 in said cancer cell test sample indicates said cancer cell is sensitive to USP1 inhibition.

In some embodiments, provided is a method of determining the sensitivity of a cancer cell to USP1 inhibition (e.g., inhibition with a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof), comprising the steps of:
a) detecting a mutation in a gene encoding BRCA2 (e.g., a loss of function mutation) in a cancer cell test sample (e.g., in a cancer sample obtained from a subject);
b) wherein presence of mutations in a gene encoding BRCA2 in said cancer cell test sample indicates said cancer cell is sensitive to USP1 inhibition.

In some embodiments, provided is a method of determining the sensitivity of a cancer cell to USP1 inhibition (e.g., inhibition with a compound of Formula (I), (II), (IIa), (IIa1), (IIb), (IIb1), (IIc), (IIIc1), (IIc′), (IIc1′) or a compound of Table 1 or a pharmaceutically acceptable salt, hydrate, solvate, prodrug, stereoisomer or tautomer thereof), comprising the steps of:
a) detecting deficiency in homologous recombination (e.g., as measured by a positive homologous recombination deficiency (HRD) score) in a cancer cell test sample (e.g., in a cancer sample obtained from a subject)
b) wherein presence of homologous recombination deficiency in said cancer cell test sample indicates said cancer cell is sensitive to USP1 inhibition.

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

Therapeutic Methods for Treating Subjects Having or Having been Diagnosed with Cancer

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of
a) detecting levels of RAD18 and/or UBE2K (e.g., RAD18 and/or UBE2K protein and/or RAD18 and/or UBE2K mRNA) in a test cancer sample (e.g., in a cancer test sample obtained from the subject);
b) comparing the test cancer sample with reference cells (e.g., a reference sample taken from a non-cancerous or normal control subject), wherein elevated levels of RAD18 and/or UBE2K in said test cancer sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (II), (IIc), (IIc1), (IIc′), (IIc1′) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting levels of translesion synthesis (e.g., detecting PCNA monoubiquitination levels) in a test cancer sample (e.g., in a cancer test sample obtained from the subject);
b) comparing the test cancer sample with reference cells (e.g., a reference sample taken from a non-cancerous or normal control subject), wherein elevated translesion synthesis (e.g., increased PCNA monoubiquitination levels) in said test cancer sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of
a) detecting mutations in a gene encoding ATM (i.e, loss function mutations) in a test cancer sample (e.g., in a cancer test sample obtained from the subject);
b) wherein presence of mutations in a gene encoding ATM in said test cancer sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting germline or somatic mutations in a gene encoding BRCA1 (e.g., a loss of function mutation) in a subject test sample (e.g., in a cancer test sample or blood test sample obtained from the subject);
b) wherein presence of mutations in a gene encoding BRCA1 in said test sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting germline or somatic mutations in a gene encoding BRCA2 (e.g., a loss of function mutation) in a subject test sample (e.g., in a cancer test sample or blood test sample obtained from the subject);
b) wherein presence of mutations in a gene encoding BRCA2 in said test sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting deficiency in homologous recombination (e.g., as measured by a positive homologous recombination deficiency (HRD) score) in a subject test sample (e.g., in a cancer sample or blood sample obtained from the subject)
b) wherein presence of homologous recombination deficiency in said test sample indicates that the subject will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject identified in step b).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

### Therapeutic Methods for Treating Cancer

In some embodiments provided is a therapeutic method of treating a cancer (e.g., a cancer associated with USP1) in a subject in need thereof comprising the steps of:
a) detecting levels of RAD18 and/or UBE2K (e.g., RAD18 and/or UBE2K protein and/or RAD18 and/or UBE2K mRNA) a cancer test sample (e.g., in a cancer sample obtained from the subject)
b) comparing the cancer test sample with a reference (e.g., a reference sample taken from a non-cancerous or normal control subject), wherein elevated levels of RAD18 and/or UBE2K in said test sample indicates that the subject's cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb), (IIc), (Ic1), (IIc′), (IIc1′) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting levels of translesion synthesis (e.g., detecting PCNA monoubiquitination levels) in a test cancer sample (e.g., in a cancer sample obtained from the subject) b) comparing the test cancer sample with a reference (e.g., a reference sample taken from a non-cancerous or normal control subject), wherein elevated translesion synthesis (e.g., increased PCNA monoubiquitination levels) in said test cancer sample indicates that the subject's cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting mutations in a gene encoding ATM (i.e, loss function mutations) in a test cancer sample (e.g., in a cancer sample obtained from the subject)
b) wherein presence of mutations in a gene encoding ATM in said cancer sample indicates that the subject's cancer will respond to therapeutic treatment with a USP1 inhibitor (e.g., a compound of Formula (I), (II), (IIa), (IIa1), (IIb), (IIb1), (IIc), (IIc1), (IIc′), (IIc′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting germline or somatic mutations in a gene encoding BRCA1 (e.g., a loss of function mutation) in a cancer subject test sample (e.g., in a cancer sample or blood sample obtained from the cancer subject)
b) wherein presence of mutations in a gene encoding BRCA1 in said test sample indicates that the subject's cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (II), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of
a) detecting germline or somatic mutations in a gene encoding BRCA2 (e.g., a loss of function mutation) in a cancer subject test sample (e.g., in a cancer sample or blood sample obtained from the cancer subject)
b) wherein presence of mutations in a gene encoding BRCA2 in said test sample indicates that the subject's cancer will respond to therapeutic treatment with a USP1 inhibitor (e.g., a compound of Formula (I), (II), (IIa), (IIa1), (IIb), (IIb), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of
a) detecting deficiency in homologous recombination (e.g., as measured by a positive homologous recombination deficiency (HRD) score) in a cancer subject test sample (e.g., in a cancer sample or blood sample obtained from the cancer subject)
b) wherein presence of homologous recombination deficiency in said test sample indicates that the subject's cancer will respond to therapeutic treatment with a USP1 inhibitor (e.g., a compound of Formula (I), (II), (IIa), (IIa1), (IIb), (IIb1), (IIc), (IIc1), (IIc′), (IIc1′) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II), (IIa), (IIa1), (IIb), (IIb1), (IIc), (IIc′), (IIc′), (IIc1′) or a compound of Table 1 or a pharmaceutically acceptable salt, hydrate, solvate, prodrug, stereoisomer or tautomer thereof) to the subject whose cancer was identified in step b).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

### Sample Preparation

The disclosure further provides assays for the detection of levels of translesion synthesis (e.g., PCNA monoubiquitination levels, levels of RAD18, (e.g., RAD18 protein and/or RAD18 mRNA), UBE2K (e.g., UBE2K protein and/or UBE2K mRNA)). The disclosure further provides assays for detecting ATM mutations (e.g. ATM loss of function mutations), loss of ATM protein expression (e.g., as measured by immunohistochemistry), BRCA1 mutations (e.g., BRCA1 loss of function mutations), BRCA2 mutations (e.g., BRCA2 loss of function mutations), BRCA1/2 deficiency and deficiencies in homologous recombination (e.g., as measured by a positive homologous recombination deficiency (HRD) score). They detection of any of the above parameters can be performed in a patient sample. e.g., in a body fluid such as blood (e.g., serum or plasma) bone marrow, cerebral spinal fluid, peritoneal/pleural fluid, lymph fluid, ascite, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

### Measurement of Gene Expression

In some embodiments, elevated levels of RAD18 and/or UBE2K are elevated RAD18 and/or UBE2K gene expression levels. In some embodiments, elevated levels of RAD18 and/or UBE2K are elevated RAD18 and/or UBE2K mRNA levels. Measurement of gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In some embodiments, gene expression is detected and quantitated by hybridization to a probe that specifically hybridizes to the appropriate probe for that biomarker. The probes also can be attached to a solid support for use in high throughput screening assays using methods known in the art.

In some embodiments, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). How-ever, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In some embodiments, the hybridized nucleic acids are detected by detecting one or more labels attached to the sample nucleic acids. The labels can be incorporated by any of a number of means well known to those of skill in the art. However, in some embodiments, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine.green fluorescent protein, and the like), radiolabels (e.g., 3H, 125I, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

In some embodiments, the detection of elevated of RAD18 and/or UBE2K mRNA levels is by quantitative reverse transcriptase (RT)-polymerase chain reaction (PCR), RNA-Seq, or microarray.

### Detection of Polypeptides

Protein levels of RAD18 and/or UBE2K can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

In some embodiments, the detection of elevated RAD18 and/or UBE2K protein levels is by Western blot. In some embodiments, the detection of elevated RAD18 and/or UBE2K protein levels is by fluorescence-activated cell sorting (FACS). In some embodiments, the detection of elevated RAD18 and/or UBE2K protein levels is by immunohistochemistry.

### Other Detection Methods

Mutations in targets of interest (e.g., BRCA1 mutations, BRCA2 mutations, ATM mutations) can be detected by methods known to those of skill in the art.

For detection of germline mutation, DNA sequencing may be performed using DNA extract from body fluid such as blood (e.g., serum or plasma) bone marrow, cerebral spinal fluid, peritoneal/pleural fluid, lymph fluid, ascite, serous fluid, sputum, lacrimal fluid, stool, and urine.

Alternatively, sequencing may be performed on DNA extracted from a tissue such as a tumor tissue. The tumor tissue can be fresh tissue or preserved tissue (e.g., formalin fixed tissue. e.g. paraffin-embedded tissue). Sequencing may also be performed using cell-free DNA. The coding regions and sometimes adjacent regions (e.g., introns, promoter) of genes of interest are sequenced using next generation sequencing (NGS) or Sanger sequencing (Genetic/Familial High-Risk Assessment: Breast, Ovarian, and Pancreatic, Version 2.2021, NCCN Clinical Practice Guidelines in Oncology, ESMO guideline for BRCA testing DOI: 10.1093/annonc/mdw327, Clinical testing of BRCA1 and BRCA2; a worldwide snapshot of technological practices). Loss of function mutations or gene rearrangements may be detected or validated using secondary methods such as qPCR, PCR, immunohistochemistry, Sanger sequencing, comparative genomic hybridization, or the PacBio system.

Deficiencies in homologous recombination can be identified by methods known to those of skill in the art. One indicator of homologous recombination deficiencies is genomic instability (e.g., represented by a positive homologous recombination deficiency (HRD) score), which can be quantified by methods known in the art (see, e.g., Pikor L, et al., Cancer Metastasis Rev. 2013; 32(3-4):341-352). HRD score is measured using next generation sequencing of DNA extracted from tumor tissues (fresh or FFPE), based on genomic instability (e.g., loss of heterozygosity, telomeric allelic imbalance, and large-scale state transitions). Commercial FDA-approved assays are available for such measures (Myriad and Foundation Medicine).

### Kits

In some embodiments kits related to methods disclosed herein are provided.

In some embodiments, a kit for predicting the sensitivity of a subject having or having been diagnosed with a disease or disorder associated with USP1 for treatment with a USP1 inhibitor is provided. The kit comprises: i) reagents capable of detecting human cancer cells associated with a disease or disorder associated with USP1 (e.g., reagents capable of specifically detecting RAD18 and/or UBE2K) and ii) instructions for how to use said kit.

In some embodiments, the present disclosure provides kit, comprising: (a) a pharmaceutical composition comprising a USP1 inhibitor and one or more pharmaceutically acceptable excipients, and (b) a diagnostic kit comprising at least one agent capable of specifically detecting RAD18 and/or UBE2K.

In some embodiments, the agent capable of specifically detecting RAD18 and/or UBE2K is capable of specifically hybridizing to RAD18 and/or UBE2K mRNA. In some embodiments, the agent capable of specifically detecting RAD18 and/or UBE2K is capable of specifically binding to RAD18 and/or UBE2K protein.

In another embodiment, the present disclosure provides kits which comprise a compound disclosed herein (or a composition comprising a compound disclosed herein) packaged in a manner that facilitates their use to practice methods of the present disclosure. In some embodiments, the kit includes a compound disclosed herein (or a composition comprising a compound disclosed herein) packaged in a container, such as a sealed bottle or vessel, with a label affixed to the container or included in the kit that describes use of the compound or composition to practice the method of the disclosure. In some embodiments, the compound or composition is packaged in a unit dosage form. The kit further can include a device suitable for administering the composition according to the intended route of administration. In some embodiments, the present disclosure provides a kit which comprise a compound disclosed herein, or a pharmaceutically acceptable salt or solvate thereof, and instructions for administering the compound, or a pharmaceutically acceptable salt or solvate thereof, to a patient having cancer.

### Selected Embodiments

Embodiment 1. A compound of Formula (I) or a pharmaceutically acceptable salt, hydrate, solvate, prodrug, stereoisomer, or tautomer thereof;

wherein:

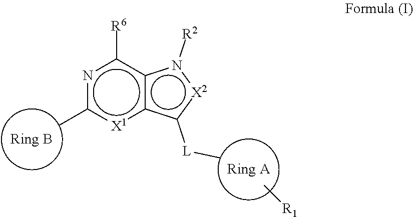

X¹ is selected from CH and N;
X² is selected from CR^{Xc2} and NR^{Xn2};
Ring B is a 5-6 member optionally substituted monocyclic aryl or heteroaryl.

L is selected from —O—, —NRⁿ—, —S—, —S(═O)—, —S(═O)₂— and —CR^{c}R^{c′}—;
Ring A is selected from C₆-C₁₀ aryl, 5-10 membered heteroaryl and 3-10 membered heterocyclyl, with 0, 1 or 2 instances of halo (e.g., —F) or -Me;
R¹ is an optionally substituted 5-10 membered heteroaryl; R² is absent or is selected from H, —C₁-C₆ alkyl, —C₃-C₉ cycloalkyl (e.g., cyclopropyl), —C₁-C₆ haloalkyl, —C₁-C₆ heteroalkyl, —C₁-C₆ hydroxyalkyl, arylalkyl, —S(═O)₂C₁-C₆alkyl (e.g., —S(═O)₂CH₃) and —C(═O)C₁-C₆alkyl (e.g., —C(═O)CH₃), wherein each hydrogen of the alkyl, haloalkyl, heteroalkyl, hydroxylalkyl and arylalkyl can be independently replaced with a deuterium atom;
R⁶ is selected from H, -D, —CN, halo, —C₁-C₆ alkyl, —C₁-C₆ heteroalkyl, —C₁-C₆ haloalkyl, —C₃-C₁₀ cycloalkyl, —OH, and —O(C₁-C₆ alkyl);
each R^{Xc2} is independently selected from H, -D, halo, —C₁-C₆ alkyl, —C₁-C₆ heteroalkyl, —NH₂, —NH(C₁-C₆ alkyl), —O(C₁-C₆ alkyl) and —N(C₁-C₆ alkyl)₂;
each R^{Xn2} is absent or independently selected from H, —C₁-C₆ alkyl —C₁-C₆ haloalkyl (e.g., CH₂CF₃), —S(═O)₂C₁-C₆alkyl (e.g., —S(═O)₂CH₃) and —C(═O)C₁-C₆alkyl (e.g., —C(═O)CH₃);
each Rⁿ is independently selected from H and —C₁-C₆ alkyl (e.g., -Me); and each R^{c} and R^{c′} is independently selected from H, —C₁-C₆ alkyl (e.g., Me), —C₁-C₆ heteroalkyl, —C₁-C₆ haloalkyl, —OH, and —O(C₁-C₆ alkyl) (e.g., —OMe), or R^{c} and R^{c′} can be taken together with the atom to which they are attached to form a —C₃-C₉ cycloalkyl (e.g., cyclopropyl) or a carbonyl.

Embodiment 2. The compound of embodiment 1 or a pharmaceutically acceptable salt, hydrate, solvate, prodrug, stereoisomer, or tautomer thereof, wherein Ring B is substituted with 0, 1, 2 or 3 instances of R^{b}, wherein
each R^{b} is independently selected from D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{b1}, —N(R^{b1})₂, —C(═O)R^{b1}, —C(═O)OR^{b1}, —NR^{b1}C(═O)R^{b1}, —NR^{b1}C(═O)OR^{b1}, —C(═O)N(R^{b1})₂, —OC(═O)N(R^{b1})₂, —S(═O)R^{b1}, —S(═O)₂R^{b1}, —SR^{b1}, —S(═O)(═NR^{b1})R^{b1}, —NR^{b1}S(═O)₂R^{b1} and —S(═O)₂N(R^{b1})₂ wherein each alkyl, cycloalkyl, heterocyclyl, aryl, heteroaryl, arylalkyl and heteroarylalkyl is optionally substituted at any available position;
each R^{b1} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl, wherein each hydrogen of the —C₁-C₆ alkyl of R^{b1} can be independently replaced with a deuterium atom.

Embodiment 3. The compound of embodiment 2 or a pharmaceutically acceptable salt, hydrate, solvate, prodrug, stereoisomer, or tautomer thereof, wherein Ring B is substituted with 0, 1 or 2 instances of R^{b}.

Embodiment 4. The compound of embodiment 2 or a pharmaceutically acceptable salt, hydrate, solvate, prodrug, stereoisomer, or tautomer thereof, wherein Ring B is substituted with 1 or 2 instances of R^{b}.

Embodiment 5. The compound of any one of embodiments 1 to 4, or a pharmaceutically acceptable salt, hydrate, solvate, prodrug, stereoisomer, or tautomer thereof, wherein each R^{b} is independently selected from halo, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{b} and —N(R^{b1})₂, wherein each alkyl, cycloalkyl and heterocyclyl is substituted with 0, 1, 2 or 3 instances of halo (e.g., —F, Cl), —OH, CN, -Me, -Et, —NH₂ or oxo and wherein each R^{b1} is independently selected from H, —C₁-C₆ alkyl, —C₁-C₆ heteroalkyl, —C₁-C₆ haloalkyl and C₃-C₉ cycloalkyl, wherein each hydrogen of the —C₁-C₆ alkyl of R^{b1} can be independently replaced with a deuterium atom.

Embodiment 6. The compound of any one of embodiments 1 to 4, or a pharmaceutically acceptable salt, hydrate, solvate, prodrug, stereoisomer, or tautomer thereof, wherein each R^{b} is independently selected from halo (e.g., —F, —Cl), —C₁-C₆ alkyl (e.g., -Me, -Et, —Pr. -ⁱPr, -ⁿBu, -sec-Bu, -iso-Bu, -^{t}Bu), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₂, —CHF₂, —CH₂CF₃)—C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrolidinyl, piperidinyl, piperazinyl, morpholinyl, 6-oxa-1-azaspiro[3.3]heptanyl, 6-oxa-1-azaspiro[3.4]octanyl), —OR^{b1} and —N(R^{b1})₂, wherein each alkyl, cycloalkyl and heterocyclyl is substituted with 0, 1, 2 or 3 instances of halo (e.g., —F, Cl), and wherein each R^{b1} is independently selected from H, —C₁-C₆ alkyl (e.g., -Me, -Et —Pr, -ⁱPr, -ⁿ Bu, -sec-Bu, -iso-Bu, -^{t}Bu), —C₁-C₆ haloalkyl (e.g., —CF₃, —CH₂F, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

Embodiment 7. The compound of any one of embodiments 1 to 4, or a pharmaceutically acceptable salt, hydrate, solvate, prodrug, stereoisomer, or tautomer thereof, wherein each R^{b} is independently selected from —Cl, -ⁱPr, —CH₂N(CH₃)CH₂CH₃, —CH₂N(CH₃)₂, —CF₃, —CH₂OH, —CH(OH)(CH₃)₂, cyclopropyl (substituted with 0, 1 or 2 instances of —F), azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OCH₂F, —OⁱPr, —OPr, —OMe, —OCD₃, —OEt, —OH, —Ocyclopropyl, —N(Me)₂, —NHMe and —NHⁱPr.

Embodiment 8. The compound of any one of embodiments 1 to 7 or a pharmaceutically acceptable salt, hydrate, solvate, prodrug, stereoisomer, or tautomer thereof, wherein Ring B is a 5-membered heteroaryl containing 1-3 heteroatoms independently selected from O, N and S.

Embodiment 9. The compound of any one of embodiments 1 to 7 or a pharmaceutically acceptable salt, hydrate, solvate, prodrug, stereoisomer, or tautomer thereof, wherein Ring B is pyrazolyl (e.g., pyrazol-5yl).

Embodiment 10. The compound of any one of embodiments 1 to 7 or a pharmaceutically acceptable salt, hydrate, solvate, prodrug, stereoisomer, or tautomer thereof, wherein Ring B is a 6 membered heteroaryl containing 1-3 nitrogen atoms.

Embodiment 11. The compound of embodiment 10 or a pharmaceutically acceptable salt, hydrate, solvate, prodrug, stereoisomer, or tautomer thereof, wherein Ring B is selected from pyridinyl, pyrimidinyl, pyrazinyl, triazinyl and pyridazinyl.

Embodiment 12. The compound of embodiment 10 or a pharmaceutically acceptable salt, hydrate, solvate, prodrug, stereoisomer, or tautomer thereof, wherein Ring B is selected from pyridinyl and pyrimidinyl.

Embodiment 13. The compound of any one of embodiments 1 to 7 or a pharmaceutically acceptable salt, hydrate, solvate, prodrug, stereoisomer, or tautomer thereof, wherein Ring B is selected from phenyl, pyrazolyl, pyridinyl and pyrimidinyl.

Embodiment 14. The compound of any one of embodiments 1 to 7 or a pharmaceutically acceptable salt, hydrate, solvate, prodrug, stereoisomer, or tautomer thereof, wherein Ring B is phenyl.

Embodiment 15. A compound of Formula (II) or a pharmaceutically acceptable salt, hydrate, solvate, prodrug, stereoisomer, or tautomer thereof;

wherein:

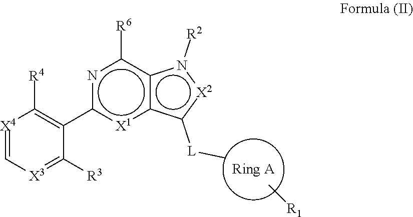

X¹ is selected from CH and N;
X² is selected from CR^{Xc2} and NR^{Xn2},
X³ is selected from CH and N;
X⁴ is selected from CH and N;
L is selected from —O—, —NRⁿ—, —S—, —S(═O)—, —S(═O)₂— and —CR^{c}R^{c′};
Ring A is selected from C₆-C₁₀ aryl, 5-10 membered heteroaryl and 3-10 membered heterocyclyl, each substituted with 0, 1 or 2 instances of halo (e.g., —F) or -Me;
R¹ is an optionally substituted 5-10 membered heteroaryl;
R² is absent or is selected from H, —C₁-C₆ alkyl, —C₃-C₉ cycloalkyl (e.g., cyclopropyl), —C₁-C₆ heteroalkyl, —C₁-C₆ haloalkyl, —C₁-C₆ hydroxyalkyl, arylalkyl, —S(═O)₂C₁-C₆ alkyl (e.g., —S(═O)₂CH₃) and —C(═O)C₁-C₆ alkyl (e.g., —C(═O)CH₃), wherein each hydrogen of the alkyl, haloalkyl, heteroalkyl, hydroxylalkyl and arylalkyl can be independently replaced with a deuterium atom;
R³ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3} S(═O)₂R^{3a} and —S(═O)₂N(R^{a3})₂ wherein each alkyl, cycloalkyl, heterocyclyl, aryl, heteroaryl, arylalkyl and heteroarylalkyl is optionally substituted at any available position;
R⁴ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4} S(═O)₂R^{4a} and —S(═O)₂N(R^{a4})₂ wherein each alkyl, cycloalkyl, heterocyclyl, aryl, heteroaryl, arylalkyl and heteroarylalkyl is optionally substituted at any available position;
each R^{Xc2} is independently selected from H, —C₁-C₆ alkyl, —C₁-C₆ heteroalkyl, —NH₂, —NH(C₁-C₆ alkyl) and —N(C₁-C₆ alkyl)₂;
each R^{Xn2} is absent or independently selected from H, —C₁-C₆ alkyl, —C₁-C₆ haloalkyl (e.g., CH₂CF₃), —S(═O)₂C₁-C₆alkyl (e.g., —S(═O)₂CH₃) and —C(═O)C₁-C₆alkyl (e.g., —C(═O)CH₃);
each Rⁿ is independently selected from H and —C₁-C₆ alkyl;
each R^{c} and R^{c′} is independently selected from H, —C₁-C₆ alkyl (e.g., -Me), —C₁-C₆ heteroalkyl, —C₁-C₆ haloalkyl, —OH, and —O(C₁-C₆ alkyl) (e.g., —OMe), or R^{c} and R can be taken together with the atom to which they are attached to form a —C₃-C₉ cycloalkyl (e.g., cyclopropyl) or a carbonyl; and
each R^{a} and R^{a3} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each hydrogen of the —C₁-C₆ alkyl can be independently replaced with a deuterium atom.

Embodiment 16. The compound of any one of embodiments 1 to 15 or a pharmaceutically acceptable salt, hydrate, solvate, prodrug, stereoisomer, or tautomer thereof, wherein X¹ is CH.

Embodiment 17. Tc compound of any one of embodiments 1 to 15 or a pharmaceutically acceptable salt, hydrate, solvate, prodrug, stereoisomer, or tautomer thereof, wherein X¹ is N.

Embodiment 18. The compound of any one of embodiments 1 to 17 or a pharmaceutically acceptable salt, hydrate, solvate, prodrug, stereoisomer, or tautomer thereof, wherein X² is CR^{Xc2}.

Embodiment 19. The compound of any one of embodiments 1 to 17 or a pharmaceutically acceptable salt, hydrate, solvate, prodrug, stereoisomer, or tautomer thereof, wherein X² is NR^{Xn2}.

Embodiment 20. The compound of any one of embodiments 1 to 17 or a pharmaceutically acceptable salt, hydrate, solvate, prodrug, stereoisomer, or tautomer thereof, wherein the compound is of Formula (IIa), wherein:

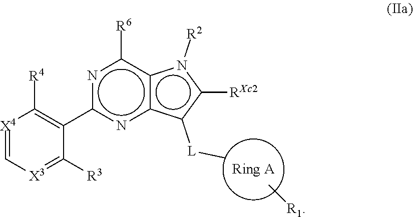

Embodiment 21. The compound of any one of embodiments 1 to 17 or a pharmaceutically acceptable salt, hydrate, solvate, prodrug, stereoisomer, or tautomer thereof, wherein the compound is of Formula (IIb), wherein:

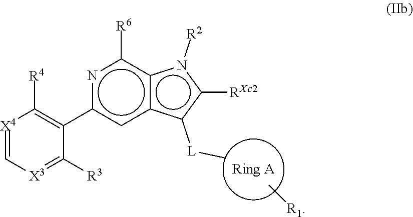

Embodiment 22. The compound of any one of embodiments 1 to 21 or a pharmaceutically acceptable salt, hydrate, solvate, prodrug, stereoisomer, or tautomer thereof, wherein each R^{Xc2} is independently selected from H, —F, -Me, —CH₂NMe₂, —CH₂NHMe, —CH₂OMe and —CH₂CH₂OMe.

Embodiment 23. The compound of embodiment 22 or a pharmaceutically acceptable salt, hydrate, solvate, prodrug, stereoisomer, or tautomer thereof, wherein each R^{Xc2} is H.

Embodiment 24. The compound of any one of embodiments 1 to 17 or a pharmaceutically acceptable salt, hydrate, solvate, prodrug, stereoisomer, or tautomer thereof, wherein the compound is of formula (IIc), wherein:

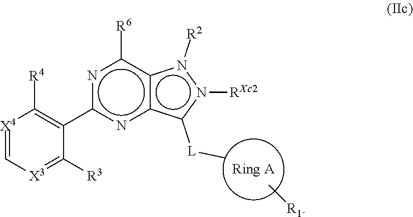

Embodiment 25. The compound of embodiment 24 or a pharmaceutically acceptable salt, hydrate, solvate, prodrug, stereoisomer, or tautomer thereof, wherein R^{Xn2} is absent or is selected from —H, -Me, —CH₂CF₃, S(═O)₂Me, —C(═O)Me.

Embodiment 26. The compound of embodiment 24 or a pharmaceutically acceptable salt, hydrate, solvate, prodrug, stereoisomer, or tautomer thereof, w % herein R^{Xn2} is absent.

Embodiment 27. The compound of embodiment 24 or a pharmaceutically acceptable salt, hydrate, solvate, prodrug, stereoisomer, or tautomer thereof, wherein R^{Xn2} is -Me.

Embodiment 28. The compound of embodiment 24 or a pharmaceutically acceptable salt, hydrate, solvate, prodrug, stereoisomer, or tautomer thereof, wherein R^{Xn2} is —H.

Embodiment 29. The compound of any one of embodiments 15 to 28 or a pharmaceutically acceptable salt, hydrate, solvate, prodrug, stereoisomer, or tautomer thereof, wherein X³ is CH.

Embodiment 30. The compound of any one of embodiments 15 to 28 or a pharmaceutically acceptable salt, hydrate, solvate, prodrug, stereoisomer, or tautomer thereof, wherein X³ is N.

Embodiment 31. The compound of any one of embodiments 15 to 30 or a pharmaceutically acceptable salt, hydrate, solvate, prodrug, stereoisomer, or tautomer thereof, wherein X⁴ is CH.

Embodiment 32. The compound of any one of embodiments 15 to 30 or a pharmaceutically acceptable salt, hydrate, solvate, prodrug, stereoisomer, or tautomer thereof, wherein X⁴ is N.

Embodiment 33. The compound of any one of embodiments 15 to 28 or a pharmaceutically acceptable salt, hydrate, solvate, prodrug, stereoisomer, or tautomer thereof, wherein the moiety represented by

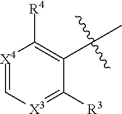

is selected from:

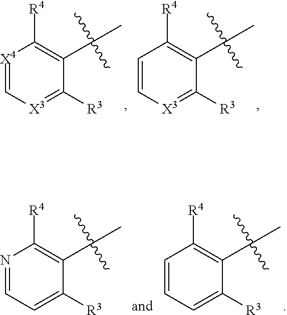

Embodiment 34. The compound of any one of embodiments 15 to 33, or a pharmaceutically acceptable salt, hydrate, solvate, prodrug, stereoisomer, or tautomer thereof, wherein each R³ is independently selected from H, D, halo, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{a3} and —N(R^{a3})₂, wherein each alkyl, cycloalkyl and heterocyclyl is substituted with 0, 1, 2 or 3 instances of halo (e.g., —F, Cl), —OH, CN, -Me, -Et, —NH₂ or oxo and wherein each R^{a3} is independently selected from H, —C₁-C₆ alkyl, —C₁-C₆ heteroalkyl, —C₁-C₆ haloalkyl and C₃-C₉ cycloalkyl, wherein each hydrogen atom of the C₁-C₆ alkyl of R^{a3} can be independently replaced by deuterium.

Embodiment 35. The compound of any one of embodiments 15 to 33, or a pharmaceutically acceptable salt, hydrate, solvate, prodrug, stereoisomer, or tautomer thereof, wherein each R³ is independently selected from H, -D, halo, —C₁-C₆ alkyl (e.g., -Me, -Et, —Pr, -ⁱPr, -ⁿBu, -sec-Bu, -iso-Bu, -^{t}Bu), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃)—C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrolidinyl, piperidinyl, piperazinyl, morpholinyl, 6-oxa-1-azaspiro[3.3]heptanyl, 6-oxa-1-azaspiro[3.4]octanyl), —OR^{a3} and —N(R^{a3})₂, wherein each alkyl, cycloalkyl and heterocyclyl is substituted with 0, 1, 2 or 3 instances of halo (e.g., —F, Cl), and wherein each RV is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl), wherein each hydrogen atom of the C₁-C₆ alkyl of R^{a3} can be independently replaced by deuterium.

Embodiment 36. The compound of any one of embodiments 15 to 33, or a pharmaceutically acceptable salt, hydrate, solvate, prodrug, stereoisomer, or tautomer thereof, wherein each R³ is independently selected from H, -D, Cl, -ⁱPr, —CH₂N(CH₃)CH₂CH₃, —CH₂N(CH₃)₂, —CF₃, —CH₂OH, cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OCH₂F, —OⁱPr, —OPr, —OMe, —OCD₃, OEt, —OH, —Ocyclopropyl, —N(Me)₂, —NHMe and —NHⁱPr.

Embodiment 37. The compound of any one of embodiments 15 to 33, or a pharmaceutically acceptable salt, hydrate, solvate, prodrug, stereoisomer, or tautomer thereof, wherein R³ is —OMe.

Embodiment 38. The compound of any one of embodiments 15 to 37 or a pharmaceutically acceptable salt, hydrate, solvate, prodrug, stereoisomer, or tautomer thereof, wherein each R⁴ is independently selected from H, D, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{a4} and —N(R^{a4})₂, wherein each alkyl, cycloalkyl and heterocyclyl is substituted with 0, 1, 2 or 3 instances of halo (e.g., —F, Cl), —OH, CN, -Me, -Et, —NH₂ or oxo and wherein each R^{a4} is independently selected from H, —C₁-C₆ alkyl, —C₁-C₆ heteroalkyl, —C₁-C₆ haloalkyl and C₃-C₉ cycloalkyl;

Embodiment 39. The compound of any one of embodiments 15 to 37, or a pharmaceutically acceptable salt, hydrate, solvate, prodrug, stereoisomer, or tautomer thereof, wherein each R⁴ is independently selected from H, D, —C₁-C₆ alkyl (e.g., -Me, -Et, —Pr, -ⁱPr, -ⁿBu, -sec-Bu, -iso-Bu, -^{t}Bu), —C₁-C₆ heteroalkyl (e.g., —CH₂NHCH₂CH₃, —CH₂N(CH₃)CH₂CH₃, —CH₂N(CH₄)₂), —C₁-C₆ haloalkyl (e.g., —CF₃, —CHF₂, —CH₂CF₃)—C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrolidinyl, piperidinyl, piperazinyl, morpholinyl, 6-oxa-1-azaspiro[3.3]heptanyl, 6-oxa-1-azaspiro[3.4]octanyl), —OR^{a4} and —N(R^{a4})₂, wherein each alkyl, cycloalkyl and heterocyclyl is substituted with 0, 1, 2 or 3 instances of halo (e.g., —F, Cl), and wherein each R⁴ is independently selected from H, —C₁-C₆ alkyl (e.g., -Me, -Et, —Pr, -ⁱPr. -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

Embodiment 40. The compound of any one of embodiments 15 to 37, or a pharmaceutically acceptable salt, hydrate, solvate, prodrug, stereoisomer, or tautomer thereof, wherein each R⁴ is independently selected from H, -D, -ⁱPr, —CH₂N(CH₃)CH₂CH₃, —CH₂N(CH₃)₂, —CF₃, —CH₂OH, cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OⁱPr, —OPr, —OMe, —OH, —Ocyclopropyl, —N(Me)₂, —NHMe and —NHⁱPr.

Embodiment 41. The compound of any one of embodiments 15 to 37, or a pharmaceutically acceptable salt, hydrate, solvate, prodrug, stereoisomer, or tautomer thereof, wherein each R⁴ is selected from H and cyclopropyl.

Embodiment 42. The compound of any one of embodiments 15 to 37, or a pharmaceutically acceptable salt, hydrate, solvate, prodrug, stereoisomer, or tautomer thereof, wherein each R⁴ is cyclopropyl.

Embodiment 43. The compound of any one of embodiments 15 to 37, or a pharmaceutically acceptable salt, hydrate, solvate, prodrug, stereoisomer, or tautomer thereof, wherein each R⁴ is H.

Embodiment 44. The compound of any one of embodiments 15 to 43 or a pharmaceutically acceptable salt, hydrate, solvate, prodrug, stereoisomer, or tautomer thereof, wherein the moiety represented by

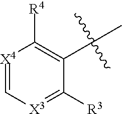

is

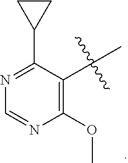 [SMILES: *c1c(OC)ncnc1C1CC1]

Embodiment 45. The compound of any one of embodiments 15 to 43 embodiment or a pharmaceutically acceptable salt, hydrate, solvate, prodrug, stereoisomer, or tautomer thereof, wherein the moiety represented by

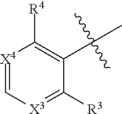

is selected from

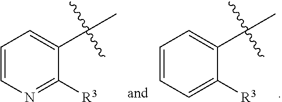

Embodiment 46. The compound of embodiment 45 or a pharmaceutically acceptable salt, hydrate, solvate, prodrug, stereoisomer, or tautomer thereof, wherein the moiety represented by

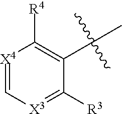

is

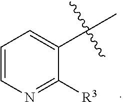

Embodiment 47. The compound of embodiment 46 or a pharmaceutically acceptable salt, hydrate, solvate, prodrug, stereoisomer, or tautomer thereof, wherein R³ is selected from cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OⁱPr, —OPr, —OMe, —OH, —Ocyclopropyl, —N(Me)₂, —NHMe and —NHⁱPr.

Embodiment 48. The compound of embodiment 45 or a pharmaceutically acceptable salt, hydrate, solvate, prodrug, stereoisomer, or tautomer thereof, wherein the moiety represented by

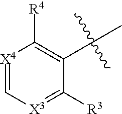

is

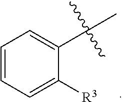

Embodiment 49. The compound of embodiment 48 or a pharmaceutically acceptable salt, hydrate, solvate, prodrug, stereoisomer, or tautomer thereof, wherein R³ is selected from -ⁱPr, —CH₂N(CH₃)CH₂CH₃, —CH₂N(CH₃)₂, —CF, —CH₂OH and cyclopropyl.

Embodiment 50. The compound of any one of embodiments 1 to 49 or a pharmaceutically acceptable salt, hydrate, solvate, prodrug, stereoisomer, or tautomer thereof, wherein L is —O—.

Embodiment 51. The compound of any one of embodiments 1 to 49 or a pharmaceutically acceptable salt, hydrate, solvate, prodrug, stereoisomer, or tautomer thereof, wherein L is —NRⁿ—.

Embodiment 52. The compound of embodiment 51 or a pharmaceutically acceptable salt, hydrate, solvate, prodrug, stereoisomer, or tautomer thereof, wherein Rⁿ is selected from H and Me.

Embodiment 53. The compound of embodiment 51 or a pharmaceutically acceptable salt, hydrate, solvate, prodrug, stereoisomer, or tautomer thereof, wherein Rⁿ is H.

Embodiment 54. The compound of any one of embodiments 1 to 49 or a pharmaceutically acceptable salt, hydrate, solvate, prodrug, stereoisomer, or tautomer thereof, wherein L is —S—.

Embodiment 55. The compound of any one of embodiments 1 to 49 or a pharmaceutically acceptable salt, hydrate, solvate, prodrug, stereoisomer, or tautomer thereof, wherein L is —S(═O)—.

Embodiment 56. The compound of any one of embodiments 1 to 49 or a pharmaceutically acceptable salt, hydrate, solvate, prodrug, stereoisomer, or tautomer thereof, wherein L is —S(═O)₂—.

Embodiment 57. The compound of any one of embodiments 1 to 49 or a pharmaceutically acceptable salt, hydrate, solvate, prodrug, stereoisomer, or tautomer thereof, wherein L is —CR^{c}R^{c′}—.

Embodiment 58. The compound of embodiment 57 or a pharmaceutically acceptable salt, hydrate, solvate, prodrug, stereoisomer, or tautomer thereof, wherein R^{c} and R^{c′} are each independently selected from H, Me, —OH, —OMe or are taken together to form a carbonyl group or a cyclopropyl group.

Embodiment 59. The compound of any one of embodiments 1 to 58 or a pharmaceutically acceptable salt, hydrate, solvate, prodrug, stereoisomer, or tautomer thereof, wherein Ring A is a C₆-C₁₀ aryl or a 3-10 membered heterocyclyl containing 1 or 2 heteroatoms selected from N, O and S, each substituted with 0, 1 or 2 instances of halo (e.g., —F) or -Me.

Embodiment 60. The compound of any one of embodiments 1 to 58 or a pharmaceutically acceptable salt, hydrate, solvate, prodrug, stereoisomer, or tautomer thereof, wherein Ring A is phenyl substituted with 0, 1 or 2 instances of halo (e.g., —F) or -Me.

Embodiment 61. The compound of any one of embodiments 1 to 58 or a pharmaceutically acceptable salt, hydrate, solvate, prodrug, stereoisomer, or tautomer thereof, wherein Ring A is phenyl substituted with 0, 1 or 2 instances of —F or -Me.

Embodiment 62. The compound of any one of embodiments 1 to 58 or a pharmaceutically acceptable salt, hydrate, solvate, prodrug, stereoisomer, or tautomer thereof, wherein Ring A a 3-10 membered heterocyclyl containing 1 or 2 heteroatoms selected from N, O and S.

Embodiment 63. The compound of embodiment 62 or a pharmaceutically acceptable salt, hydrate, solvate, prodrug, stereoisomer, or tautomer thereof, wherein Ring A is selected from piperidinyl and piperazinyl.

Embodiment 64. The compound of any one of embodiments 1 to 58 or a pharmaceutically acceptable salt, hydrate, solvate, prodrug, stereoisomer, or tautomer thereof, wherein the moiety represented by

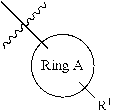

is selected from

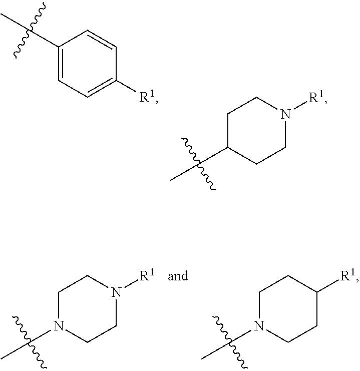

each independently substituted with 0, 1 or 2 instances of halo (e.g., —F) or-Me.

Embodiment 65. The compound of any one of embodiments 1 to 58 or a pharmaceutically acceptable salt, hydrate, solvate, prodrug, stereoisomer, or tautomer thereof, wherein the moiety represented by

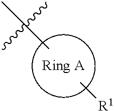

is

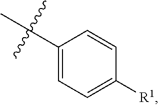

wherein the phenyl is further substituted with 0, 1 or 2 instances of halo (e.g., —F) or -Me.

Embodiment 66. The compound of embodiment 65, wherein the phenyl is further substituted with 0, 1 or 2 instances of —F or -Me.

Embodiment 67. The compound of any one of embodiments 1 to 58 or a pharmaceutically acceptable salt, hydrate, solvate, prodrug, stereoisomer, or tautomer thereof, wherein the moiety represented by

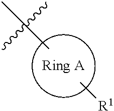

is selected from

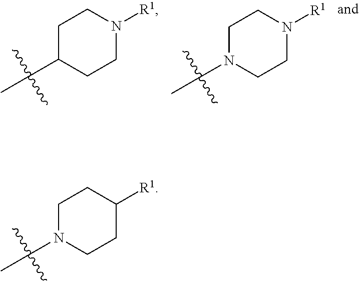

Embodiment 68. The compound of any one of embodiments 1 to 67 or a pharmaceutically acceptable salt, hydrate, solvate, prodrug, stereoisomer, or tautomer thereof, wherein R¹ is a 5-10 member heteroaryl substituted with 0, 1 or 2 instances of R⁵, wherein each R⁵ is independently selected from halo, —CN, —C₁-C₆ alkyl including deuterated versions thereof, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R⁵)₂ wherein each alkyl, cycloalkyl, heterocyclyl, aryl, heteroaryl, arylalkyl and heteroarylalkyl is optionally substituted at any available position and wherein each R¹ is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl.

Embodiment 69. The compound of embodiment 68 or a pharmaceutically acceptable salt, hydrate, solvate, prodrug, stereoisomer, or tautomer thereof, wherein R¹ is a 5-6 member monocyclic heteroaryl containing 1-3 heteroatoms selected from O, N and S.

Embodiment 70. The compound of of embodiment 68 or a pharmaceutically acceptable salt, hydrate, solvate, prodrug, stereoisomer, or tautomer thereof, wherein R¹ is a 5 member monocyclic heteroaryl containing 1-3 heteroatoms selected from O, N and S.

Embodiment 71. The compound of of embodiment 68 or a pharmaceutically acceptable salt, hydrate, solvate, prodrug, stereoisomer, or tautomer thereof, wherein R¹ is selected from pyrolyl, pyrazolyl, imidazolyl, thiazolyl, furanyl, thiophenyl, oxazolyl, thiadiazolyl, oxadiazolyl, each substituted with 0, 1 or 2 instances of R⁵.

Embodiment 72. The compound of of embodiment 68 or a pharmaceutically acceptable salt, hydrate, solvate, prodrug, stereoisomer, or tautomer thereof, wherein R¹ is selected from pyrazolyl and imidazolyl, each substituted with 0, 1 or 2 instances of R⁵.

Embodiment 73. The compound of of embodiment 68 or a pharmaceutically acceptable salt, hydrate, solvate, prodrug, stereoisomer, or tautomer thereof, wherein R¹ is pyrazolyl (e.g., pyrazol-1-yl) substituted with 0, 1 or 2 instances of R⁵¹.

Embodiment 74. The compound of of embodiment 68 or a pharmaceutically acceptable salt, hydrate, solvate, prodrug, stereoisomer, or tautomer thereof, wherein R¹ is imidazolyl (e.g., imidazol-2-yl) substituted with 0, 1 or 2 instances of R⁵.

Embodiment 75. The compound of any one of embodiments 68 to 74 or a pharmaceutically acceptable salt, hydrate, solvate, prodrug, stereoisomer, or tautomer thereof, wherein R⁵ is selected from CN, —C₁-C₆ alkyl (e.g., -Me, -CD₃, -Et, —Pr, -ⁱPr, -ⁿBu, -^{t}Bu), —C₁-C₆ haloalkyl (e.g., —CF₃, —CHF₂, —CH₂CF₃), —O(C₁-C₆ alkyl) (e.g., —OMe, —OEt), —C₃-C₁₀ cycloalkyl (e.g., cyclopropyl, cyclobutyl, cyclopentyl, cyclohexyl, cycloheptyl) and 3-10 membered heterocyclyl (e.g., azetidinyl, oxetanyl, tetrahydrofuranyl, tetrahydropyranyl, piperazinyl, piperidinyl, morpholinyl), wherein each alkyl, cycloalkyl and heterocyclyl is substituted with 0, 1 or 2 instances of-Me, —OMe, —OH, CN, halo (e.g., —F, Cl).

Embodiment 76. The compound of any one of embodiments 68 to 74 or a pharmaceutically acceptable salt, hydrate, solvate, prodrug, stereoisomer, or tautomer thereof, wherein R⁵ is selected from CN, -Me, -CD₃, -Et, -ⁱPr, —CF₃, —OMe, —OEt, cyclopropyl, oxetanyl (e.g., oxetan-3-yl) and azetidinyl (e.g., N-methyl-azetidin-3-yl).

Embodiment 77. The compound of any one of embodiments 1 to 76 or a pharmaceutically acceptable salt, hydrate, solvate, prodrug, stereoisomer, or tautomer thereof, wherein R¹ is selected from:

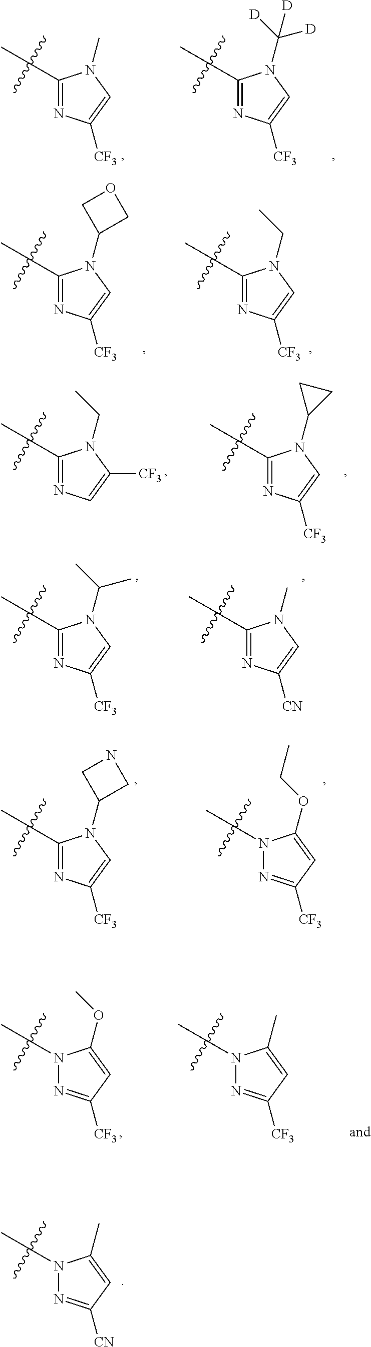

Embodiment 78. The compound of any one of embodiments 1 to 77 or a pharmaceutically acceptable salt, hydrate, solvate, prodrug, stereoisomer, or tautomer thereof, wherein R² is absent.

Embodiment 79. The compound of any one of embodiments 1 to 77 or a pharmaceutically acceptable salt, hydrate, solvate, prodrug, stereoisomer, or tautomer thereof, wherein R² is selected from H, -Me, -CD₃, -n-butyl, —CH₂CF₃, —S(═O)₂Me, —C(═O)Me, cyclopropyl, —CH₂CH₂OMe, —CH₂CH₂OH and benzyl.

Embodiment 80. The compound of any one of embodiments 1 to 77 or a pharmaceutically acceptable salt, hydrate, solvate, prodrug, stereoisomer, or tautomer thereof, wherein R² is selected from Me, —CH₂CH₂OMe, —CH₂CH₂OH and benzyl.

Embodiment 81. The compound of any one of embodiments 1 to 77 or a pharmaceutically acceptable salt, hydrate, solvate, prodrug, stereoisomer, or tautomer thereof, wherein R² is —C₁-C₆ alkyl wherein one or more of the hydrogen atoms of the alkyl are replaced with a deuterium atom. (e.g., -CD3, CD2CD3).

Embodiment 82. The compound of embodiment 81 or a pharmaceutically acceptable salt, hydrate, solvate, prodrug, stereoisomer, or tautomer thereof, wherein R² is -CD3.

Embodiment 83. The compound of any one of embodiments 1 to 82 or a pharmaceutically acceptable salt, hydrate, solvate, prodrug, stereoisomer, or tautomer thereof, wherein R² is H or -Me.

Embodiment 84. The compound of any one of embodiments 1 to 83 or a pharmaceutically acceptable salt, hydrate, solvate, prodrug, stereoisomer, or tautomer thereof, wherein R⁶ is selected from H, D, —CN, halo (e.g., —F, —Cl), —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CF₃), —OH, and —O(C₁-C₆ alkyl) (e.g., —OMe).

Embodiment 85. The compound of any one of embodiments 1 to 83 or a pharmaceutically acceptable salt, hydrate, solvate, prodrug, stereoisomer, or tautomer thereof, wherein R⁶ is selected from H, -D, —CN, —F, —Cl, -Me, -Et, —Pr, -ⁱPr, -ⁿBu, -^{t}Bu, —CF₃, —CHF₂, —OH and —OMe).

Embodiment 86. The compound of any one of embodiments 1 to 83 or a pharmaceutically acceptable salt, hydrate, solvate, prodrug, stereoisomer, or tautomer thereof, wherein R⁶ is selected from H, —F, Me and —OMe.

Embodiment 87. The compound of any one of embodiments 1 to 83 or a pharmaceutically acceptable salt, hydrate, solvate, prodrug, stereoisomer, or tautomer thereof, wherein R⁶ is H.

Embodiment 88. The compound of any one of embodiments 1 to 87 or a pharmaceutically acceptable salt, hydrate, solvate, prodrug, stereoisomer, or tautomer thereof, wherein the compound is selected from the compounds of Table 1.

Embodiment 89. A pharmaceutical composition comprising a compound of any one of embodiments 1 to 88 or a pharmaceutically acceptable salt, hydrate, solvate, prodrug, stereoisomer, or tautomer thereof and a pharmaceutically acceptable carrier.

Embodiment 90. The pharmaceutical composition of embodiment 89, further comprising a second therapeutic agent.

Embodiment 91. A method for treating or preventing a disease or disorder associated with the inhibition of USP1 comprising administering to a patient in need thereof an effective amount of a compound of any one of embodiments 1 to 88 or a pharmaceutically acceptable salt, hydrate, solvate, prodrug, stereoisomer, or tautomer thereof.

Embodiment 92. A method of treating a disease or disorder associated with the inhibition of USP1 comprising administering to a patient in need thereof an effective amount (e.g., a therapeutically effective amount) of a compound of any one of embodiments 1 to 88 or a pharmaceutically acceptable salt, hydrate, solvate, prodrug, stereoisomer, or tautomer thereof.

Embodiment 93. A method for inhibiting USP1 comprising administering to a patient in need thereof an effective amount of a compound of any one of embodiments 1 to 88 or a pharmaceutically acceptable salt, hydrate, solvate, prodrug, stereoisomer, or tautomer thereof.

Embodiment 94. A method for treating or preventing cancer in a patient in need thereof comprising administering to the patient in need thereof an effective amount of a compound of any one of embodiments 1 to 88 or a pharmaceutically acceptable salt, hydrate, solvate, prodrug, stereoisomer, or tautomer thereof.

Embodiment 95. A method for treating cancer in a patient in need thereof comprising administering to the patient in need thereof an effective amount (e.g., a therapeutically effective amount) of a compound of any one of embodiments 1 to 88 or a pharmaceutically acceptable salt, hydrate, solvate, prodrug, stereoisomer, or tautomer thereof.

Embodiment 96. The method of embodiment 94 or 95 wherein the cancer is a dedifferentiated ID-driven cancer.

Embodiment 97. The method of any one of embodiments 94 to 96 wherein the cancer is a cancer that is sensitive to USP1 inhibition.

Embodiment 98. The method of any one of embodiments 94 to 97 wherein the cancer is a cancer that is sensitive to USP1 inhibition due to a dysfunctional DNA-repair pathway.

Embodiment 99. The method of any one of embodiments 94 to 98 wherein the cancer is a HRR (homologous recombination repair) gene mutant cancer.

Embodiment 100. The method of any one of embodiments 94 to 99 wherein the cancer is a HRR (homologous recombination repair) gene mutant cancer selected from the group consisting of ATM, BARD1, BRCA1, BRCA2, BRIP1, CDK12, CHEK1, CHEK2, FANCL, PALB2, PPP2R2A, RAD51B, RAD51C, RAD51D, or RAD54L mutant cancer.

Embodiment 101. The method of any one of embodiments 94 to 100 wherein the cancer is characterized by elevated levels of translesion synthesis (e.g., a cancer characterized by elevated levels of RAD18 and/or UBE2K, a cancer characterized by elevated PCNA monoubiquitination).

Embodiment 102. The method of any one of embodiments 94 to 101 wherein the cancer is characterized by a deficiency in homologous recombination (e.g., a positive homologous recombination deficiency (HRD) score).

Embodiment 103. The method of any one of embodiments 94 to 102 wherein the cancer is a BRCA1 and/or a BRCA2 mutant cancer.

Embodiment 104. The method of any one of embodiments 94 to 103 wherein the cancer is a BRCA1 and/or a BRCA2 deficient cancer.

Embodiment 105. The method of any one of embodiments 94 to 104 wherein the cancer is an ATM mutant cancer.

Embodiment 106. The method of any one of embodiments 94 to 105 wherein the cancer is an BARD1 mutant cancer.

Embodiment 107. The method of any one of embodiments 94 to 106 wherein the cancer is an BRIP1 mutant cancer.

Embodiment 108. The method of any one of embodiments 94 to 107 wherein the cancer is an CDK12 mutant cancer.

Embodiment 109. The method of any one of embodiments 94 to 108 wherein the cancer is an CHEK1 mutant cancer.

Embodiment 110. The method of any one of embodiments 94 to 109 wherein the cancer is an CHEK2 mutant cancer.

Embodiment 111. The method of any one of embodiments 94 to 110 wherein the cancer is an FANCL mutant cancer.

Embodiment 112. The method of any one of embodiments 94 to 111 wherein the cancer is an PALB2 mutant cancer.

Embodiment 113. The method of any one of embodiments 94 to 112 wherein the cancer is an PPP2R2A mutant cancer.

Embodiment 114. The method of any one of embodiments 94 to 113 wherein the cancer is an RAD51B mutant cancer.

Embodiment 115. The method of any one of embodiments 94 to 114 wherein the cancer is an RAD51C mutant cancer.

Embodiment 116. The method of any one of embodiments 94 to 115 wherein the cancer is an RAD5ID mutant cancer.

Embodiment 117. The method of any one of embodiments 94 to 116 wherein the cancer is an RAD54L mutant cancer

Embodiment 118. The method of any one of embodiments 94 to 117 wherein the cancer is a PARP inhibitor resistant or refractory cancer.

Embodiment 119. The method of any one of embodiments 94 to 118 wherein the cancer is selected from adrenocortical carcinoma. AIDS-related lymphoma, AIDS-related malignancies, anal cancer, cerebellar astrocytoma, extrahepatic bile duct cancer, bladder cancer osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g., renal pelvis and ureter), retinoblastoma, rhabdomyosarcoma, salivary gland cancer, malignant fibrous histiocytoma of bone, soft tissue sarcoma, Sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor.

Embodiment 120. The method of any one of embodiments 94 to 119 wherein the cancer can be any cancer in any organ, for example, a cancer selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma. CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

Embodiment 121. The method of any one of embodiments 94 to 119 wherein the cancer is selected from liposarcoma, neuroblastoma, glioblastoma, bladder cancer, adrenocortical cancer, multiple myeloma, colorectal cancer, non-small cell lung cancer, Human Papilloma Virus-associated cervical, oropharyngeal, penis, anal, thyroid or vaginal cancer or Epstein-Barr Virus-associated nasopharyngeal carcinoma, gastric cancer, rectal cancer, thyroid cancer, breast cancer, prostate cancer, ovarian cancer, pancreatic cancer, Hodgkin lymphoma and diffuse large B-cell lymphoma.

Embodiment 122. The method of any one of embodiments 94 to 119 wherein the cancer is selected from breast cancer (e.g., triple negative breast cancer (TNBC)), ovarian cancer (e.g., platinum-resistant ovarian cancer, platinum-refractory ovarian cancer), pancreatic cancer, prostate cancer and lung cancer (e.g., non-small cell lung cancer (NSCLC)).

Embodiment 123. The method of any one of embodiments 94 to 104 wherein the cancer is selected from breast cancer (e.g., triple negative breast cancer (TNBC)), ovarian cancer (e.g., platinum-resistant ovarian cancer, platinum-refractory ovarian cancer), prostate cancer and lung cancer (e.g., non-small cell lung cancer (NSCLC)).

Embodiment 124. The method of any one of embodiments 94 to 119 wherein the cancer is breast cancer.

Embodiment 125. The method of any one of embodiments 94 to 119 wherein the cancer is triple negative breast cancer (TNBC).

Embodiment 126. The method of any one of embodiments 94 to 119 wherein the cancer is ovarian cancer.

Embodiment 127. The method of embodiment 126, wherein the cancer is platinum-resistant ovarian cancer.

Embodiment 128. The method of embodiment 126, wherein the cancer is platinum-refractory ovarian cancer.

Embodiment 129. The method of any one of embodiments 94 to 119 wherein the cancer is prostate cancer.

Embodiment 130. The method of any one of embodiments 94 to 119 wherein the cancer is lung cancer.

Embodiment 131. The method of any one of embodiments 94 to 119 wherein the cancer is non-small cell lung cancer (NSCLC)

Embodiment 132. A method for treating or preventing a disease or disorder associated with DNA damage comprising administering to a patient in need of a treatment for diseases or disorders associated with DNA damage an effective amount of a compound of any one of embodiments 1 to 88 or a pharmaceutically acceptable salt, hydrate, solvate, prodrug, stereoisomer, or tautomer thereof.

Embodiment 133. The method of embodiment 132, wherein the disease is cancer.

Embodiment 134. A method for treating a disease or disorder associated with DNA damage comprising administering to a patient in need of a treatment for diseases or disorders associated with DNA damage an effective amount (e.g., a therapeutically effective amount) of a compound of any one of embodiments 1 to 88 or a pharmaceutically acceptable salt, hydrate, solvate, prodrug, stereoisomer, or tautomer thereof.

Embodiment 135. A method of inhibiting, modulating or reducing DNA repair activity exercised by USP1 comprising administering to a patient in need thereof an effective amount of a compound of any one of embodiments 1 to 88 or a pharmaceutically acceptable salt, hydrate, solvate, prodrug, stereoisomer, or tautomer thereof.

Embodiment 136. A compound of any one of embodiments 1-88 or a pharmaceutically acceptable salt, hydrate, solvate, prodrug, stereoisomer, or tautomer thereof for use in a method for treating or preventing a disease or disorder associated with the inhibition of USP1, wherein the method comprises administering to a patient in need thereof an effective amount of the compound.

Embodiment 137. A compound of any one of embodiments 1-88 or a pharmaceutically acceptable salt, hydrate, solvate, prodrug, stereoisomer, or tautomer thereof for use in a method of treating a disease or disorder associated with the inhibition of USP1 comprising administering to a patient in need thereof an effective amount (e.g., a therapeutically effective amount) of the compound.

Embodiment 138. A compound of any one of embodiments 1-88 or a pharmaceutically acceptable salt, hydrate, solvate, prodrug, stereoisomer, or tautomer thereof for use in a method for inhibiting USP1 comprising administering to a patient in need thereof an effective amount of the compound.

Embodiment 139. A compound of any one of embodiments 1-88 or a pharmaceutically acceptable salt, hydrate, solvate, prodrug, stereoisomer, or tautomer thereof for use in a method for treating or preventing cancer in a patient in need thereof comprising administering to the patient in need thereof an effective amount of the compound.

Embodiment 140. A compound of any one of embodiments 1-88 or a pharmaceutically acceptable salt, hydrate, solvate, prodrug, stereoisomer, or tautomer thereof for use in a method for treating cancer in a patient in need thereof comprising administering to the patient in need thereof a therapeutically effective amount (e.g., a therapeutically effective amount) of the compound.

Embodiment 141. The compound for use of embodiment 139 or 140 wherein the cancer is a dedifferentiated ID-driven cancer.

Embodiment 142. The compound for use of any one of embodiments 139 to 141 wherein the cancer is a cancer that is sensitive to USP1 inhibition.

Embodiment 143. The compound for use of any one of embodiments 139 to 142 wherein the cancer is a cancer that is sensitive to USP1 inhibition due to a dysfunctional DNA-repair pathway.

Embodiment 144. The compound for use of any one of embodiments 139 to 143 wherein the cancer is a HRR (homologous recombination repair) gene mutant cancer.

Embodiment 145. The compound for use of any one of embodiments 139 to 144 wherein the cancer is a HRR (homologous recombination repair) gene mutant cancer selected from the group consisting of ATM, BARD1. BRCA1, BRCA2, BRIP1, CDK12, CHEK1, CHEK2, FANCL, PALB2, PPP2R2A, RAD51B, RAD51C, RAD51D, or RAD54L mutant cancer.

Embodiment 146. The compound for use of any one of embodiments 139 to 145 wherein the cancer is characterized by elevated levels of translesion synthesis (e.g., a cancer characterized by elevated levels of RAD18 and/or UBE2K, a cancer characterized by elevated PCNA monoubiquitination).

Embodiment 147. The compound for use of any one of embodiments 139 to 146 wherein the cancer is characterized by a deficiency in homologous recombination (e.g., a positive homologous recombination deficiency (HRD) score).

Embodiment 148. The compound for use of any one of embodiments 139 to 147 wherein the cancer is a BRCA1 and/or a BRCA2 mutant cancer.

Embodiment 149. The compound for use of any one of embodiments 139 to 148 wherein the cancer is a BRCA1 and/or a BRCA2 deficient cancer.

Embodiment 150. The compound for use of any one of embodiments 139 to 149 wherein the cancer is an ATM mutant cancer.

Embodiment 151. The compound for use of any one of embodiments 139 to 150 wherein the cancer is an BARD1 mutant cancer.

Embodiment 152. The compound for use of any one of embodiments 139 to 151 wherein the cancer is an BRIP1 mutant cancer.

Embodiment 153. The compound for use of any one of embodiments 139 to 152 wherein the cancer is an CDK12 mutant cancer.

Embodiment 154. The compound for use of any one of embodiments 139 to 153 wherein the cancer is an CHEK1 mutant cancer.

Embodiment 155. The compound for use of any one of embodiments 139 to 154 wherein the cancer is an CHEK2 mutant cancer.

Embodiment 156. The compound for use of any one of embodiments 139 to 155 wherein the cancer is an FANCL mutant cancer.

Embodiment 157. The compound for use of any one of embodiments 139 to 156 wherein the cancer is an PALB2 mutant cancer.

Embodiment 158. The compound for use of any one of embodiments 139 to 157 wherein the cancer is an PPP2R2A mutant cancer.

Embodiment 159. The compound for use of any one of embodiments 139 158 wherein the cancer is an RAD51B mutant cancer.

Embodiment 160. The compound for use of any one of embodiments 139 to 159 wherein the cancer is an RAD51C mutant cancer.

Embodiment 161. The compound for use of any one of embodiments 139 to 160 wherein the cancer is an RAD51 D mutant cancer.

Embodiment 162. The compound for use of any one of embodiments 139 to 161 wherein the cancer is an RAD54L mutant cancer.

Embodiment 163. The compound for use of any one of embodiments 139 to 162 wherein the cancer is a PARP inhibitor resistant or refractory cancer.

Embodiment 164. The compound for use of any one of embodiments 139 to 163 wherein the cancer is selected from adrenocortical carcinoma, AIDS-related lymphoma, AIDS-related malignancies, anal cancer, cerebellar astrocytoma, extrahepatic bile duct cancer, bladder cancer osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g., renal pelvis and ureter), retinoblastoma, rhabdomyosarcoma, salivary gland cancer, malignant fibrous histiocytoma of bone, soft tissue sarcoma, Sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor.

Embodiment 165. The compound for use of any one of embodiments 139 to 163 wherein the cancer can be any cancer in any organ, for example, a cancer selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

Embodiment 166. The compound for use of any one of embodiments 139 to 163 wherein the cancer is selected from liposarcoma, neuroblastoma, glioblastoma, bladder cancer, adrenocortical cancer, multiple myeloma, colorectal cancer, non-small cell lung cancer, Human Papilloma Virus-associated cervical, oropharyngeal, penis, anal, thyroid or vaginal cancer or Epstein-Barr Virus-associated nasopharyngeal carcinoma, gastric cancer, rectal cancer, thyroid cancer, breast cancer, prostate cancer, ovarian cancer, pancreatic cancer, Hodgkin lymphoma and diffuse large B-cell lymphoma.

Embodiment 167. The compound for use of any one of embodiments 139 to 163 wherein the cancer is selected from breast cancer (e.g., triple negative breast cancer (TNBC)), ovarian cancer (e.g., platinum-resistant ovarian cancer, platinum-refractory ovarian cancer), pancreatic cancer, prostate cancer and lung cancer (e.g., non-small cell lung cancer (NSCLC)).

Embodiment 168. The compound for use of any one of embodiments 139 to 163 wherein the cancer is selected from breast cancer (e.g., triple negative breast cancer (TNBC)), ovarian cancer (e.g., platinum-resistant ovarian cancer, platinum-refractory ovarian cancer), prostate cancer and lung cancer (e.g., non-small cell lung cancer (NSCLC)).

Embodiment 169. The compound for use of any one of embodiments 139 to 163 wherein the cancer is breast cancer.

Embodiment 170. The compound for use of any one of embodiments 139 to 163 wherein the cancer is triple negative breast cancer (TNBC).

Embodiment 171. The compound for use of any one of embodiments 139 to 163 wherein the cancer is ovarian cancer.

Embodiment 172. The compound for use of embodiment 171, wherein the cancer is platinum-resistant ovarian cancer.

Embodiment 173. The compound for use of embodiment 171, wherein the cancer is platinum-refractory ovarian cancer.

Embodiment 174. The compound for use of any one of embodiments 139 to 163 wherein the cancer is prostate cancer.

Embodiment 175. The compound for use of any one of embodiments 139 to 163 wherein the cancer is lung cancer.

Embodiment 176. The compound for use of any one of embodiments 139 to 163 wherein the cancer is non-small cell lung cancer (NSCLC)

Embodiment 177. A compound of any one of embodiments 1-88 or a pharmaceutically acceptable salt, hydrate, solvate, prodrug, stereoisomer, or tautomer thereof for use in a method for treating or preventing a disease or disorder associated with DNA damage comprising administering to a patient in need of a treatment for diseases or disorders associated with DNA damage an effective amount of the compound.

Embodiment 178. The compound for use of embodiment 177, wherein the disease is cancer.

Embodiment 179. A compound of any one of embodiments 1-88 or a pharmaceutically acceptable salt, hydrate, solvate, prodrug, stereoisomer, or tautomer thereof for use in a method for treating a disease or disorder associated with DNA damage comprising administering to a patient in need of a treatment for diseases or disorders associated with DNA damage an effective amount (e.g., a therapeutically effective amount) of the compound.

Embodiment 180. A compound of any one of embodiments 1-88 or a pharmaceutically acceptable salt, hydrate, solvate, prodrug, stereoisomer, or tautomer thereof for use in a method of inhibiting, modulating or reducing DNA repair activity exercised by USP1 comprising administering to a patient in need thereof an effective amount of the compound.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

### Abbreviations

### General

ADDP 1,1′-(azodicarbonyl)dipiperidine
anhy. anhydrous
aq. aqueous
satd, saturated
min(s) minute(s)
hr(s) hour(s)
mL milliliter
mmol millimole(s)
mol mole(s)
MS mass spectrometry
NMR nuclear magnetic resonance
TLC thin layer chromatography
HPLC high-performance liquid chromatography
Me methyl
i-Pr iso-propyl
t-Bu tert-butyl
^{t}BuXPhos 2-di-tert-butylphosphino-2′,4′,6′-triisopropylbiphenyl
Ph phenyl
Et ethyl
Bz benzoyl
RuPhos 2-dicyclohexylphosphino-2′,6′-diisopropoxybiphenyl

Spectrum
Hz hertz
δ chemical shift
J coupling constant
s singlet
d doublet
t triplet
q quartet
m multiplet
br broad
qd quartet of doublets
dquin doublet of quintets
dd doublet of doublets
dt doublet of triplets

### Solvents and Reagents

DAST Diethylaminosulfurtrifluoride
CHCl₃ chloroform
DCM dichloromethane
DMF dimethylformamide
Et₂O diethyl ether
EtOH ethyl alcohol
EtOAc ethyl acetate
MeOH methyl alcohol
MeCN acetonitrile
PE petroleum ether
THF tetrahydrofuran
DMSO dimethyl sulfoxide
t-BuOK potassium tert-butoxide
9-BBN 9-borabicyclo[3.3.]nonane
AcOH acetic acid
HCl hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
TFA trifluoroacetic acid
Na₂SO₄ sodium sulfate
NaBH₄ sodium borohydride
NaHCO₃sodium bicarbonate
LiHMDS lithium hexamethyldisilylamide
NaBH₄ sodium borohydride
Et₃N triethylamine
Py pyridine
PCC pyridinium chlorochromate
DMAP 4-(dimethylamino)pyridine
DIPEA N,N-diisopropylethylamine
BINAP 2,2′-bis(diphenylphosphanyl)-1,1′-binaphthyl
dppf 1,1′-bis(diphenylphosphino)ferrocene
PEP Phospho(enol)pyruvic acid
LDH Lactate Dehydrogenase
DTT DL-Dithiothreitol
BSA Bovine Serum Albumin
NADH β-Nicotinamide adenine dinucleotide, reduced
Pd(t-Bu3P)₂ bis(tri-tert-butylphosphine)palladium(0)
AcCl acetyl chloride
i-PrMgCl Isopropylmagnesium chloride
TBSCl tert-Butyl(chloro)dimethylsilane
(i-PrO)₄Ti titanium tetraisopropoxide
BHT 2,6-di-t-butyl-4-methylphenoxide
BzCl benzoyl chloride
CsF cesium fluoride
DCC dicyclohexylcarbodiimide
DMP Dess-Martin periodinane
EtMgBr ethylmagnesium bromide
EtOAc ethyl acetate
TEA triethylamine
AlaOH alanine
TBAF tetra-n-butylammonium fluoride
TBS t-butyldimethylsilyl
TMS trimethylsilyl
TMSCF₃ (Trifluoromethyl)trimethylsilane
Ts p-toluenesulfonyl
Bu butyl
Ti(OⁱPr)₄ tetraisopropoxytitanium
LAH Lithium Aluminium Hydride
LDA lithium diisopropylamide
LiOH·H₂O lithium hydroxide hydrates
MAD methyl aluminum bis(2,6-di-t-butyl-4-methylphenoxide)
NBS N-bromosuccinimide
Na₂SO₄ sodium sulfate
Na₂S₂O₃ sodium thiosulfate
MeCN acetonitrile
Boc t-butoxycarbonyl
MTBE methyl tert-butyl ether
DIAD diisopropyl azodicarboxylate

### General Experimental Notes:

In the following examples, the chemical reagents were purchased from commercial sources (such as Alfa, Acros, Sigma Aldrich, TCI and Shanghai Chemical Reagent Company), and used without further purification.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley. New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative pyrazoles that have been listed herein. The compounds provided herein may be prepared from known or commercially available starting materials and reagents by one skilled in the art of organic synthesis.

### Intermediate Synthesis

### 4-[(1-methyl-4-(trifluoromethyl)imidazol-2-yl]aniline

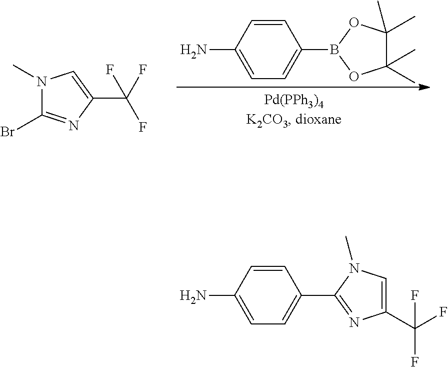 [SMILES: Cn1cc(C(F)(F)F)nc1Br ; CC1(C)OB(c2ccc(N)cc2)OC1(C)C ; Cn1cc(C(F)(F)F)nc1-c1ccc(N)cc1]

To a mixture of 2-bromo-1-methyl-4-(trifluoromethyl)imidazole (950 mg, 4.15 mmol) and 4-(4,4,5,5-tetramethyl-1,3,2-dioxaborolan-2-yl)aniline (1.36 g, 6.22 mmol) in 1,4-dioxane (15 mL) were added tetrakis(triphenylphosphine)palladium (479 mg, 415 μmol) and potassium carbonate (1.26 g, 9.13 mmol) at 100° C. The resulting mixture was stirred at 100° C. for 16 hr then cooled to 25° C. The solid was filtered out and the filtrate was concentrated under reduced pressure. The residue was purified by silica gel column chromatography, eluted with 1%-50% ethyl acetate in petroleum ether to give 4-[1-methyl-4-(trifluoromethyl)imidazol-2-yl]aniline (400 mg, 1.66 mmol, 40% yield) as a yellow oil. MS: m/z=242.15 [M+H]⁺. ¹H NMR (400 MHz, Chloroform-d) δ 7.43 (d, J=8.4 Hz, 2H), 7.27 (s, 1H), 6.75 (d, J=8.0 Hz, 2H), 3.90 (br., 2H), 3.74 (s, 3H).

### Synthesis of 2-[[3-bromo-5-(4-cyclopropyl-6-methoxy-pyrimidin-5-yl)pyrazolo[4,3-d]pyrimidin-1-yl]methoxy]ethyl-trimethyl-silane

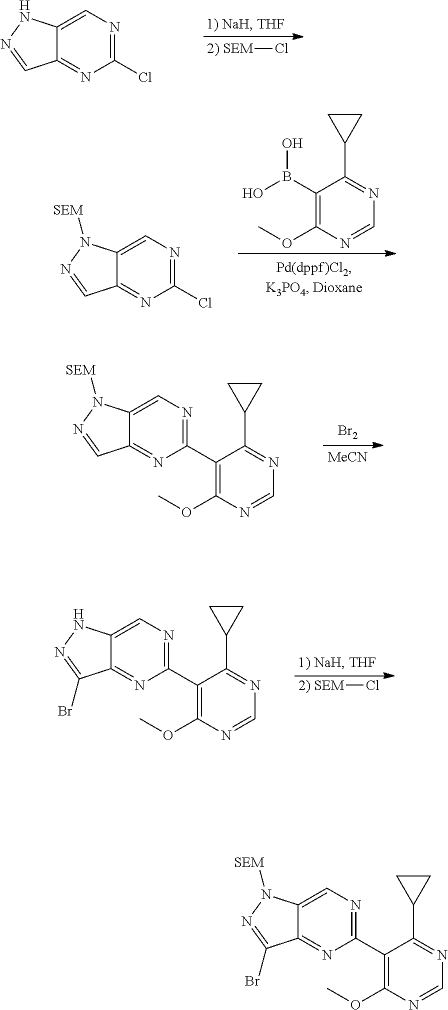

### 2-[(5-chloropyrazolo[4,3-d]pyrimidin-1-yl)methoxy]ethyl-trimethyl-silane

To a solution of 5-chloro-1H-pyrazolo[4,3-d]pyrimidine (500 mg, 3.24 mmol) in tetrahydrofuran (5 mL) was added sodium hydride (60% dispersion in mineral oil, 97 mg, 4 mmol) at 0° C. under N₂ atmosphere, and the reaction mixture was stirred at this temperature for 45 min. Then 2-(chloromethoxy)ethyl-trimethyl-silane (674 mg, 4 mmol, 716 μL) was added to the reaction mixture at 0° C., and then stirred at 25° C. for 2 h. The reaction was quenched with ammonium chloride (50 mL), and then the mixture was extracted with ethyl acetate (3×50 mL). The combined organic extracts were washed with brine (40 mL), dried over anhydrous sodium sulfate. After filtration, the filtrate was concentrated under reduced pressure. The residue was purified by silica gel column chromatography, eluted with 1˜60% ethyl acetate in petroleum ether to give 2-[(5-chloropyrazolo[4,3-d]pyrimidin-1-yl)methoxy]ethyl-trimethyl-silane (700 mg, 2.46 mmol, 76% yield) as a light-yellow oil. MS: m/z=285.05, 287.05 [M+H]⁺. ¹H NMR (400 MHz, Chloroform-d) δ 9.14 (d, J=0.8 Hz, 1H), 8.18 (d, J=0.8 Hz, 1H), 5.81 (s, 2H), 3.59-3.50 (m, 2H), 0.99-0.84 (m, 2H), −0.06 (s, 9H).

### 2-[[5-(4-cyclopropyl-6-methoxy-pyrimidin-5-yl)pyrazolo[4,3-d]pyrimidin-1-yl]methoxy]ethyl-trimethyl-silane

To a solution of 2-[(5-chloropyrazolo[4,3-d]pyrimidin-1-yl)methoxy]ethyl-trimethyl-silane (220 mg, 772.43 umol) and (4-cyclopropyl-6-methoxy-pyrimidin-5-yl)boronic acid (150 mg, 772 umol) in dioxane (5 mL) were added 1,1′-Bis(diphenylphosphino)ferrocene-palladium (II) dichloromethane complex (631 mg, 772 μmol) and potassium phosphate tribasic anhydrous (164 mg, 772 μmol) at 25° C. under N₂ atmosphere. Then the mixture was heated to 100° C., and stirred for 8 h under N₂ atmosphere. The mixture was cooled down to 25° C. Then the mixture was diluted with ethyl acetate (30 mL) and washed with brine (4×15 mL), dried over anhydrous sodium sulfate. The organic phase was concentrated under reduced pressure. The residue was purified by silica gel column chromatography, elute with 1˜70% ethyl acetate in petroleum ether to give 2-[[5-(4-cyclopropyl-6-methoxy-pyrimidin-5-yl)pyrazolo[4,3-d]pyrimidin-1-yl]methoxy]ethyl-trimethyl-silane (250 mg, 627.30 μmol, 81% yield) as a yellow oil. MS: m/z=399.25 [M+H]⁺. ¹H NMR (400 MHz, Chloroform-d) δ 9.43 (d, J=0.8 Hz, 1H), 8.71 (s, 1H), 8.35 (d, J=0.8 Hz, 1H), 5.89 (s, 2H), 3.95 (s, 3H), 3.70-3.61 (m, 2H), 1.61-1.70 (m, 1H), 1.28 (t, J=2.0 Hz, 2H), 1.02-0.89 (m, 4H), −0.01 (s, 9H).

### 3-bromo-5-(4-cyclopropyl-6-methoxy-pyrimidin-5-yl)-1H-pyrazolo[4,3-d]pyrimidine

To a solution of 2-[[5-(4-cyclopropyl-6-methoxy-pyrimidin-5-yl)pyrazolo[4,3-d]pyrimidin-1-yl]methoxy]ethyl-trimethyl-silane (250 mg, 627.30 μmol) in acetonitrile (5 mL) was added molecular bromine (401 mg, 2.5 mmol) at 25° C. under N₂ atmosphere. The reaction mixture was heated to 80° C., and stirred for 3 h. The reaction mixture was cooled to 25° C. Then the reaction was quenched with brine (150 mL), and then the mixture was extracted with ethyl acetate (3×100 mL). The combine organic was dried over anhydrous sodium sulfate. After filtration, the filtrate was concentrated under reduced pressure. The residue was purified by silica gel column chromatography, eluted with 70˜80% ethyl acetate in petroleum ether to give 3-bromo-5-(4-cyclopropyl-6-methoxy-pyrimidin-5-yl)-1H-pyrazolo[4,3-d]pyrimidine (90 mg, 259.24 μmol, 41% yield) as white solid. MS: m/z=346.95, 348.95 [M+H]⁺.

### 2-[[3-bromo-S-(4-(cyclopropyl-6-methoxy-pyrimidin-5-yl)pyrazolo[4,3-d]pyrimidin-1-yl]methoxy]ethyl-trimethyl-silane

To a solution of 3-bromo-5-(4-cyclopropyl-6-methoxy-pyrimidin-5-yl)-1H-pyrazolo[4,3-d]pyrimidine (60 mg, 172.83 μmol) in tetrahydrofuran (1 mL) was added sodium hydride (60% dispersion in mineral oil, 5.2 mg, 216 μmol) at 0° C. under N₂ atmosphere, and the reaction mixture was stirred at this temperature for 45 min. Then 2-(chloromethoxy)ethyl-trimethyl-silane (36 mg, 215 μmol, 38 μL) was added to the reaction mixture at 0° C., and then stirred at 25° C. for 2 h. The reaction was quenched with aq. ammonium chloride (20 mL), and then the mixture was extracted with ethyl acetate (3×20 mL). The combined organic extracts were washed with brine (20 mL), dried over anhydrous sodium sulfate. After filtration, the filtrate was concentrated under reduced pressure. The residue was purified by silica gel column chromatography, eluted with 1˜50% ethyl acetate in petroleum ether to give 2-[[3-bromo-5-(4-cyclopropyl-6-methoxy-pyrimidin-5-yl)pyrazolo[4,3-d]pyrimidin-1-yl]methoxy]ethyl-trimethyl-silane (70 mg, 146.6 μmol, 85% yield) as a yellow oil. MS: m/z=476.95, 478.95 [M+H]⁺.

### Synthesis of 2-(4-cyclopropyl-6-methoxy-pyrimidin-5-yl)-5H-pyrrolo[3,2-d]pyrimidine (4-cyclopropyl-6-methoxy-pyrimidin-5-yl)boronic Acid

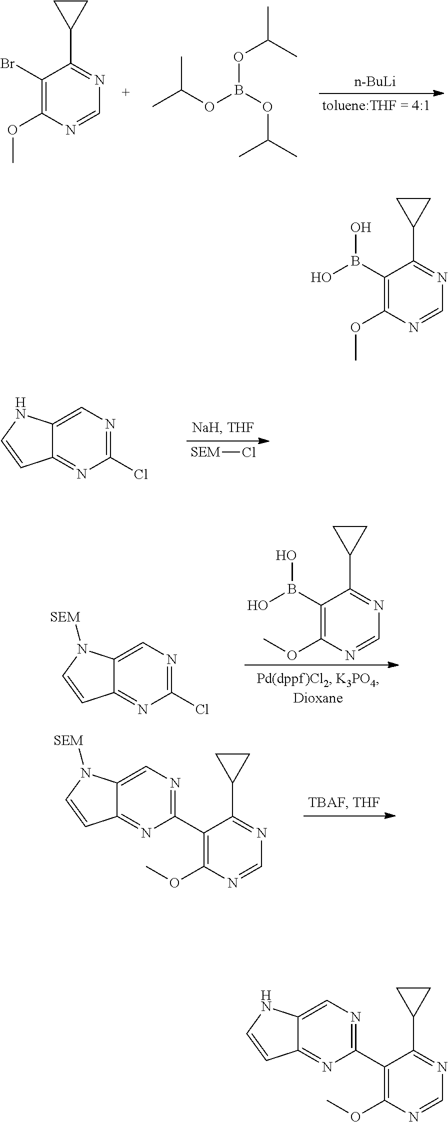

To a stirred solution of 5-bromo-4-cyclopropyl-6-methoxy-pyrimidine (4.37 g, 19.08 mmol) in toluene (40 mL) and tetrahydrofuran (10 mL) was added triisopropyl borate (4.66 g, 24.80 mmol, 5.72 mL) at room temperature under nitrogen atmosphere. The mixture was cooled down to −70° C. To the above mixture was slowly added n-butyl lithium (30.52 mmol, 2.5M in hexane, 12.21 mL) dropwise at −70° C. The resulting mixture was stirred for additional 15 min at −70° C. The reaction was quenched with 1N HCl (12 mL) at −70° C. The mixture was purified by RP-Flash with the following conditions: (Column: Spherical C18, 20-40 um, 330 g; Mobile Phase A: Water (10 mM NH₄HCO₃), Mobile Phase B: MeCN; Flow rate: 100 mL/min; Gradient (B %): 0% B to 0% B in 10 min, 95% B to 95% B in 10 min; Detector: UV 254 & 220 nm; RT: 3 min). The collected fractions were combined and concentrated under reduced pressure to afford (4-cyclopropyl-6-methoxy-pyrimidin-5-yl)boronic acid (3.5 g, 18.04 mmol, 94% yield) as a white solid. MS: m/z=195.10 [M+H]⁺. 1H NMR (400 MHz, DMSO-d6) δ 8.70 (s, 1H), 3.92 (s, 3H), 2.06-1.95 (m, 1H), 1.17-1.03 (m, 4H).

### 2-[(2-chloropyrrolo[3,2-d]pyrimidin-5-yl)methoxy]ethyl-trimethyl-silane

To a stirred mixture of 2-chloro-5H-pyrrolo[3,2-d]pyrimidine (5 g, 32.56 mmol) in tetrahydrofuran (50 mL) was added sodium hydride (859.55 mg, 35.81 mmol) under nitrogen atmosphere at 0° C. The resulting mixture was stirred for 1 hr at 0° C. To the above mixture was added 2-(Trimethylsilyl)ethoxymethyl chloride (5.43 g, 32.56 mmol) under nitrogen atmosphere at 0° C. The resulting mixture was stirred at 25° C. for 2 hr. The reaction was quenched by the addition of ammonium chloride (100 mL). The resulting mixture was extracted with ethyl acetate (3×100 mL). The combined organic layers were washed with brine (3×100 mL), dried over anhydrous sodium sulfate. After filtration, the filtrate was concentrated under reduced pressure. The residue was purified by silica gel column chromatography, eluted with 32˜33% ethyl acetate in petroleum ether to give 2-[(2-chloropyrrolo[3,2-d]pyrimidin-5-yl)methoxy]ethyl-trimethyl-silane (4.5 g, 15.85 mmol, 49% yield) as a yellow oil. MS: m/z=284.15, 286.15 [M+H]⁺. ¹H NMR (400 MHz, Chloroform-d) δ 8.83 (d, J=0.8 Hz, 1H), 7.61 (d, J=3.2 Hz, 1H), 6.66-6.65 (m, 1H), 5.54 (s, 2H), 3.50-3.46 (m, 2H), 0.92-0.90 (m, 2H), −0.03 (s, 9H).

### 2-[[2-(4-cyclopropyl-6-methoxy-pyrimidin-5-yl)pyrrolo[3,2-d]pyrimidin-5-yl]methoxy]ethyl-trimethyl-silane

To a stirred mixture of 2-[(2-chloropyrrolo[3,2-d]pyrimidin-5-yl)methoxy]ethyl-trimethyl-silane (994 mg, 3.50 mmol), (4-cyclopropyl-6-methoxy-pyrimidin-5-yl)boronic acid (815.27 mg, 4.20 mmol) and potassium phosphate (3.72 g, 17.51 mmol) in 1,4-dioxane (10 mL) was added 1,1′-bis(diphenylphosphino)ferrocene-palladium(II)dichloride dichloromethane complex (287 mg, 350 μmol) under nitrogen atmosphere at 25° C. The resulting mixture was stirred at 100° C. for 16 hr. The reaction was cooled to room temperature, then diluted with water (50 mL), extracted with dichloromethane (3×40 mL). The combined organic layers were washed with brine (30 mL), dried over anhydrous sodium sulfate. After filtration, the filtrate was concentrated under reduced pressure. The residue was purified by silica gel column chromatography, eluted with 0˜60% ethyl acetate in petroleum ether to give 2-[[2-(4-cyclopropyl-6-methoxy-pyrimidin-5-yl)pyrrolo[3,2-d]pyrimidin-5-yl]methoxy]ethyl-trimethyl-silane (480 mg, 1.21 mmol, 34% yield) as a brown solid. MS: m/z=398.20 [M+H]⁺. ¹H NMR (400 MHz, Chloroform-d) δ 9.14 (s, 1H), 8.68 (s, 1H), 7.64 (d, J=3.2 Hz, 1H), 6.81 (d, J=3.2 Hz, 1H), 5.61 (s, 2H), 3.94 (s, 3H), 3.58-3.56 (m, 2H), 1.70-1.61 (m, 1H), 1.30-1.22 (m, 4H), 1.01-0.87 (m, 2H), 0.00 (s, 9H).

### 2-(4-cyclopropyl-6-methoxy-pyrimidin-5-yl)-5H-pyrrolo[3,2-d]pyrimidine

To a stirred solution of 2-[[2-(4-cyclopropyl-6-methoxy-pyrimidin-5-yl)pyrrolo[3,2-d]pyrimidin-5-yl]methoxy]ethyl-trimethyl-silane (350 mg, 0.88 mmol) in tetrahydrofuran (4 mL) was added tetrabutyl ammonium fluoride (1 M in THF, 4.41 mL, 4.41 mmol) at 25° C. under nitrogen atmosphere. After stirred at 60° C. for 16 hours, the reaction mixture was concentrated under reduced pressure. The residue was purified by Prep-TLC (dichloromethane:methyl alcohol=10:1) to give 2-(4-cyclopropyl-6-methoxy-pyrimidin-5-yl)-5H-pyrrolo[3,2-d]pyrimidine (183 mg, 684.64 umol, 77% yield) as a yellow oil. MS: m/z=268.00 [M+H]⁺.

### Synthesis of 2-[[7-bromo-2-(2-isopropylphenyl)pyrrolo[3,2-d]pyrimidin-5-yl]methoxy]ethyl-trimethyl-silane

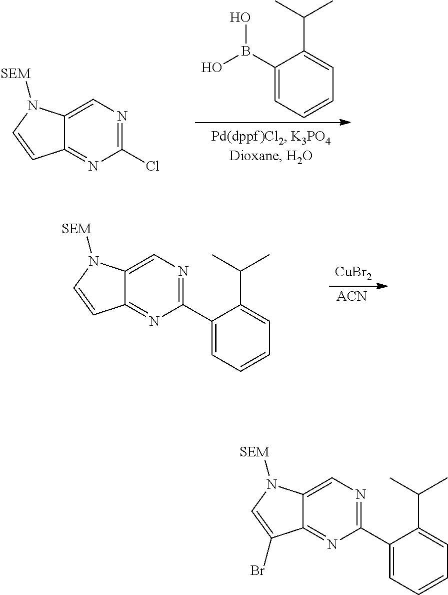

### 2-[[2-(2-isopropylphenyl)pyrrolo[3,2-d]pyrimidin-5-yl]methoxy]ethyl-trimethyl-silane

To a solution of 2-[(2-chloropyrrolo[3,2-d]pyrimidin-5-yl)methoxy]ethyl-trimethyl-silane (1 g, 3.52 mmol) and (2-isopropylphenyl) boronic acid (867 mg, 5.3 mmol) in Dioxane (5 mL) were added potassium phosphate (1.50 g, 7.05 mmol) and [1,1′-Bis(diphenylphosphino)ferrocene]dichloropalladium(II)(288 mg, 352 μmol). After the resulting mixture was stirred at 100° C. for 16 h under N₂ atmosphere, it was filtered and then concentrated under reduced pressure. The residue was purified by silica gel column chromatography, eluted with 1%-50% EA in Petroleum ether. The collected fractions were combined and concentrated under reduced pressure to give 2-[[2-(2-isopropylphenyl)pyrrolo[3,2-]pyrimidin-5-yl]methoxy]ethyl-trimethyl-silane (1.16 g, 3.17 mmol, 90% yield) as a brown oil. MS: m/z=368.15 [M+H]⁺. ¹H NMR (400 MHz, Chloroform-d) δ 9.17 (s, 1H), 7.60-7.53 (m, 2H), 7.51-7.38 (m, 2H), 7.37-7.24 (m, 1H), 6.77 (d, J=3.2 Hz, 1H), 5.59 (s, 2H), 3.60-3.52 (m, 2H), 3.50-3.40 (m, 1H), 1.33-1.20 (m, 6H), 0.98-0.94 (m, 2H).

### 2-[[7-bromo-2-(2-isopropylphenyl)pyrrolo[3,2-d]pyrimidin-5-yl]methoxy]ethyl-trimethyl-silane

To a solution of 2-[[2-(2-isopropylphenyl)pyrrolo[3,2-d]pyrimidin-5-yl]methoxy]ethyl-trimethyl-silane (400 mg, 1.09 mmol) in acetonitrile (20 mL) was added cupric bromide (729 mg, 3.3 mmol) at 0° C. After the resulting solution was stirred at 0° C. for 16 hr under N₂ atmosphere then 7 M ammonia in methanol (5 mL) was added at 0° C. for 15 min. The resulting mixture was quenched by the addition of H₂O (100 mL) at 0° C. The mixture solution was extracted with EA (3×50 mL). The combined organic fractions were dried over anhydrous Na₂SO₄. The filtrate was concentrated under reduced pressure. The residue was purified by silica gel column chromatography, eluted with 1%-50% EA in Petroleum ether. The collected fractions were combined and concentrated under reduced pressure to give 2-[[7-bromo-2-(2-isopropylphenyl)pyrrolo[3,2-d]pyrimidin-5-yl]methoxy]ethyl-trimethyl-silane (270 mg, 604.76 μmol, 55% yield) as a brown oil. MS: m/z=445.12 [M+H]⁺. ¹H NMR (400 MHz, Chloroform-d) δ 9.10 (s, 1H), 7.70-7.62 (m, 2H), 7.51-7.38 (m, 2H), 7.34-7.28 (m, 1H), 5.58 (s, 2H), 3.62-3.53 (m, 2H), 3.53-3.42 (m, 1H), 1.28 (d, J=6.8 Hz, 6H), 1.01-0.88 (m, 2H).

### 2-(2-isopropylphenyl)-N-[4-[1-methyl-4-(trifluoromethyl)imidazol-2-yl]phenyl]-5-(2-trimethylsilylethoxymethyl)pyrrolo[3,2-d]pyrimidin-7-amine

### Synthesis of 1-methyl-2-[4-[(4,4,5,5-tetramethyl-1,3,2-dioxaborolan-2-yl)methyl]phenyl]-4-(trifluoromethyl)imidazole

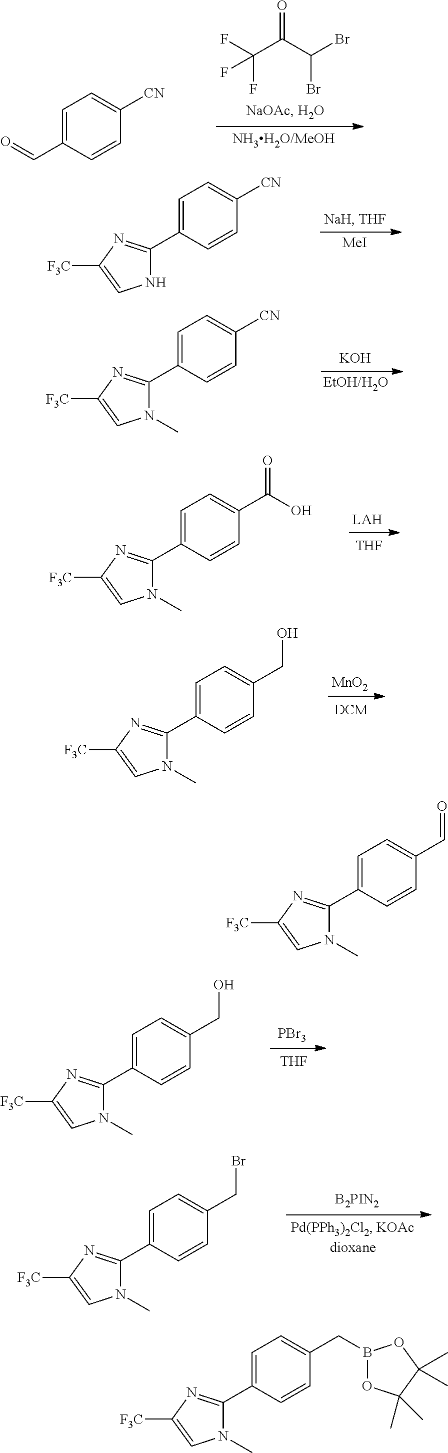

### 4-[4-(trifluoromethyl)-1H-imidazol-2-yl]benzonitrile

To a stirred solution of 3,3-dibromo-1,1,1-trifluoropropan-2-one (1.80 g, 6.67 mmol) in water (20 mL) was added sodium acetate (547 mg, 6.7 mmol) at room temperature under Ar atmosphere. The mixture was stirred at 100° C. for 45 min under Ar atmosphere. After cooling to room temperature, the mixture was added to a solution of 4-formylbenzonitrile (875 mg, 6.7 mmol) in methanol (36 mL) and NH₃·H₂O (7.20 mL). The resulting mixture was stirred at 100° C. for 1 h under Ar atmosphere. The residue was acidified to pH 7 with 1 N aq. HCl at 0° C. The resulted mixture was extracted with ethyl acetate (3×100 mL). The combined organic layers were washed with brine (3×50 mL), dried over anhydrous sodium sulfate. After filtration, the filtrate was concentrated under reduced pressure. The residue was purified by silica gel column chromatography, eluted with 30˜70% ethyl acetate in petroleum ether to give 4-[4-(trifluoromethyl)-1H-imidazol-2-yl]benzonitrile (830 mg, 3.49 mmol, 52% yield) as a light yellow solid. MS: m/z=238.05 [M+H]⁺. ¹H NMR (400 MHz, DMSO-d₆) δ 13.55 (s, 1H), 8.18-8.11 (m, 2H), 8.09-8.07 (m, 1H), 8.01-7.94 (m, 2H)

### 4-[1-methyl-4-(trifluoromethyl)imidazol-2-yl]benzonitrile

To a solution of 4-[4-(trifluoromethyl)-1H-imidazol-2-yl]benzonitrile (830 mg, 3.50 mmol) in tetrahydrofuran (10 mL) was added sodium hydride (60% in oil, 168 mg, 4.2 mmol) at 0° C. The mixture was stirred at 0° C. for 1 h. Iodomethane (497 mg, 3.50 mmol) was added at 0° C., and the mixture was allowed to warm to room temperature and stirred for 1 h. The reaction mixture was quenched by water (40 mL) and extracted with ethyl acetate (3×50 mL). The combined organic layers were dried over anhydrous sodium sulfate. After filtration, the filtrate was concentrated under reduced pressure. The residue was purified by silica gel column chromatography, eluted with 1˜50% ethyl acetate in petroleum ether to give 4-[1-methyl-4-(trifluoromethyl)imidazol-2-yl]benzonitrile (790 mg, 3.14 mmol, 90% yield) as a light yellow solid. MS: m/z=252.10 [M+H]⁺; ¹H NMR (400 MHz, Chloroform-d) δ 8.07-7.95 (m, 5H), 3.90 (s, 3H).

### 4-[1-methyl-4-(trifluoromethyl)imidazol-2-yl]benzoic Acid

To a stirred solution of 4-[1-methyl-4-(trifluoromethyl)imidazol-2-yl]benzonitrile (790 mg, 3.14 mmol) in ethanol (4.00 mL) and water (4.00 mL) was added potassium hydroxide (882 mg, 15.7 mmol) dropwise at room temperature under Ar atmosphere. The resulting mixture was stirred at 100° C. for 3 h under Ar atmosphere. The mixture was neutralized to pH 7 with saturated aqueous sodium bicarbonate (100 mL). The resulting mixture was filtered, the filter cake was washed with ethyl acetate (3×50 mL). The filtrate was concentrated under reduced pressure to give 4-[1-methyl-4-(trifluoromethyl)imidazol-2-yl]benzoic acid (720 mg, 2.66 mmol, 85% yield) as a white solid. MS: m/z=271.00 [M+H]⁺. ¹H NMR (400 MHz, DMSO-d₆) δ 13.16 (s, 1H), 8.11-8.03 (m, 2H), 8.01 (s, 1H), 7.97-7.71 (m, 2H), 3.85 (s, 3H).

### [4-[1-methyl-4-(trifluoromethyl)imidazol-2-yl]phenyl]methanol

To a stirred solution of 4-[1-methyl-4-(trifluoromethyl)imidazol-2-yl]benzoic acid (720 mg, 2.7 mmol) in tetrahydrofuran (8 mL) was added lithium aluminum hydride (126.4 mg, 3.3 mmol) at 0° C. under Ar atmosphere. The resulting mixture was stirred at 0° C. for 1 h under Ar atmosphere. The mixture was allowed to warm to room temperature and stirred for 4 h. The reaction was quenched with ice at room temperature. The resulting mixture was extracted with ethyl acetate (3×100 mL). The combined organic layers were washed with brine (3×80 mL), dried over anhydrous sodium sulfate. After filtration, the filtrate was concentrated under reduced pressure. The residue was purified by silica gel column chromatography, eluted with 1˜50% ethyl acetate in petroleum ether to give [4-[1-methyl-4-(trifluoromethyl)imidazol-2-yl]phenyl]methanol (600 mg, 2.33 mmol, 88% yield) as a colorless oil. MS: m/z=257.00 [M+H]⁺.

### 4-[1-methyl-4-(trifluoromethyl)imidazol-2-yl]benzaldehyde

To a stirred solution of [4-[1-methyl-4-(trifluoromethyl)imidazol-2-yl]phenyl]methanol (600 mg, 2.33 mmol) in dichloromethane (6 mL) was added manganese dioxide (2 g, 23.4 mmol) at room temperature under Ar atmosphere. The resulting mixture was stirred at 40° C. for 16 h under Ar atmosphere. The resulting mixture was filtered. The filtrate was concentrated under reduced pressure to give 4-[1-methyl-4-(trifluoromethyl)imidazol-2-yl]benzaldehyde (595 mg, 2.33 mmol, 99% yield) as a colorless oil. MS: m/z=255.00 [M+H]⁺. ¹H NMR (400 MHz, Chloroform-d) δ 10.11 (s, 1H), 8.09-7.99 (m, 2H), 7.88 (d, J=8.0 Hz, 2H), 7.39 (s, 1H), 3.86 (s, 3H).

### 2-[4-(bromomethyl)phenyl]-1-methyl-4-(trifluoromethyl)imidazole

To a solution of [4-[1-methyl-4-(trifluoromethyl)imidazol-2-yl]phenyl]methanol (2.4 g, 9.37 mmol) in tetrahydrofuran (10 mL) was added tribromophosphane (12.7 g, 46.8 mmol) at 25° C., and then stirred at 25° C. for 2 hr. The resulted solution was quenched by saturated aqueous sodium bicarbonate (400 mL), then was extracted with ethyl acetate (3×150 mL). The combined organic layers were dried over anhydrous sodium sulfate. After filtration, the filtrate was concentrated under reduced pressure. The residue was purified by silica gel column chromatography, eluted with 20˜40% ethyl acetate in petroleum ether to give 2-[4-(bromomethyl)phenyl]-1-methyl-4-(trifluoromethyl)imidazole (2.3 g, 7.21 mmol, 76% yield) as a white solid. MS: m/z=318.95, 320.95[M+H]⁺. 1H NMR (400 MHz, Chloroform-d) δ 7.68-7.61 (m, 2H), 7.56-7.48 (m, 2H), 7.35-7.34 (m, 1H), 4.55 (s, 2H), 3.81 (s, 3H).

### 1-methyl-2-[4-[(4,4,5,5-tetramethyl-1,3,2-dioxaborolan-2-yl)methyl]phenyl]-4-(trifluoromethyl)imidazole

To a solution of 2-[4-(bromomethyl)phenyl]-1-methyl-4-(trifluoromethyl)imidazole (500 mg, 1.57 mmol) and 4,4,5,5-tetramethyl-2-(4,4,5,5-tetramethyl-1,3,2-dioxaborolan-2-yl)-1,3,2-dioxaborolane (597 mg, 2.35 mmol) in dioxane (5 mL) were added dichloropalladium;triphenylphosphane (110 mg, 157 umol) and potassium acetate (461 mg, 4.7 mmol, 293.8 uL) at 25° C. under N₂ atmosphere. Then the mixture was heated to 80° C., and stirred for 16 h under N₂ atmosphere. The mixture was cooled down to 25° C. The resulted mixture was diluted with water (20 mL) and extracted with ethyl acetate (3×20 mL), dried over anhydrous sodium sulfate. The combined organic phase was concentrated under reduced pressure. The residue was purified by silica gel column chromatography, elute with 40-50% ethyl acetate in petroleum ether to give 1-methyl-2-[4-[(4,4,5,5-tetramethyl-1,3,2-dioxaborolan-2-yl)methyl]phenyl]-4-(trifluoromethyl)imidazole (518 mg, 1.41 mmol, 90% yield) as a white solid. MS: m/z=367.17 [M+H]⁺. ¹H NMR (400 MHz, Chloroform-d) δ 7.56-7.47 (m, 2H), 7.34-7.27 (m, 3H), 3.78 (s, 3H), 2.36 (s, 2H), 1.29 (d, J=4.0 Hz, 12H).

### Example 1. Synthesis of 2-(4-cyclopropyl-6-methoxy-pyrimidin-5-yl)-7-[4-[1-methyl-4-(trifluoromethyl)imidazol-2-yl]phenoxy]-5H-pyrrolo[3,2-d]pyrimidine (Compound 69)

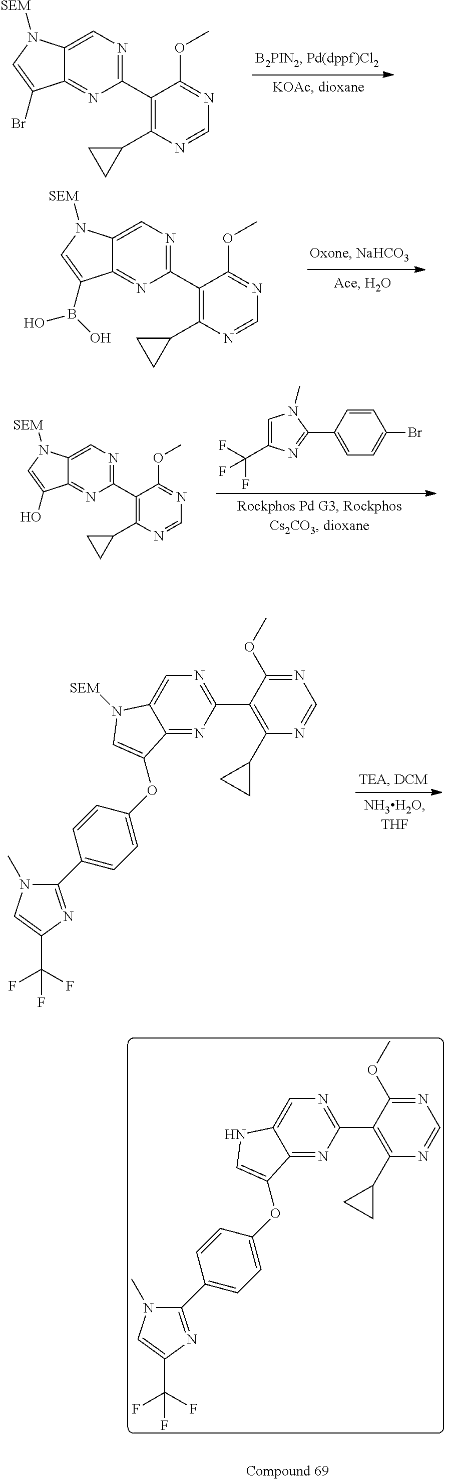

### [2-(4-cyclopropyl-6-methoxy-pyrimidin-5-yl)-5-(2-trimethylsilylethoxymethyl)pyrrolo[3,2-d]pyrimidin-7-yl]boronic Acid

To a stirred mixture of 2-[[7-bromo-2-(4-cyclopropyl-6-methoxy-pyrimidin-5-yl)pyrrolo[3,2-d]pyrimidin-5-yl]methoxy]ethyl-trimethyl-silane (600 mg, 1.26 mmol), potassium acetate (247 mg, 2.5 mmol) and bis(pinacolato)diboron (479 mg, 1.9 mmol) in 1,4-dioxane (10 mL) was added 1,1′-bis(diphenylphosphino)ferrocene-palladium(II)dichloride dichloromethane complex (103 mg, 126 μmol) under nitrogen atmosphere at 25° C. The resulting mixture was stirred at 80° C. for 16 hr. The reaction was cooled to room temperature, then diluted with water (100 mL), extracted with dichloromethane (3×20 mL). The combined organic layers were washed with brine (60 mL), dried over anhydrous sodium sulfate. After filtration, the filtrate was concentrated under reduced pressure. The resulted residue was purified by RP-Flash with the following conditions: Column: C18 spherical Column, 20-35 μm, 100A, 80 g; Mobile Phase A: Water (5 mM aq. NH₄HCO₃), Mobile Phase B: MeCN; Flow rate: 60 mL/min; Gradient: 5% B to 5% B in 5 min, 5% B to 63% B in 10 min, 63% B to 63% B in 5 min, 63% B to 95% B in 5 min; Detector: UV 254 & 210 nm; RT: 20 min. The collected fractions were combined, concentrated and then lyophilized to give [2-(4-cyclopropyl-6-methoxy-pyrimidin-5-yl)-5-(2-trimethylsilylethoxymethyl)pyrrolo[3,2-d]pyrimidin-7-yl]boronic acid (200 mg, 453.14 μmol, 36% yield) as an off-white solid. MS: m/z=442.20 [M+H]⁺. ¹H NMR (400 MHz, Chloroform-d) δ 9.19 (s, 1H), 8.71 (s, 1H), 8.01 (s, 1H), 5.63 (s, 2H), 3.95 (s, 3H), 3.59-3.55 (m, 2H), 1.77-1.73 (m, 1H), 1.30-1.25 (m, 2H), 0.97-0.89 (m, 4H), −0.01 (s, 9H).

### 2-(4-cyclopropyl-6-methoxy-pyrimidin-5-yl)-5-(2-trimethysilylethoxymethyl)pyrrolo[3,2-d]pyrimidin-7-ol

To a stirred mixture of [2-(4-cyclopropyl-6-methoxy-pyrimidin-5-yl)-5-(2-trimethylsilylethoxymethyl)pyrrolo[3,2-d]pyrimidin-7-yl]boronic acid (200 mg, 453.14 μmol) in acetone (1 mL) and water (3 mL) was added sodium bicarbonate (266 mg, 3.17 mmol) and Oxone (223 mg, 363 μmol) under nitrogen atmosphere at 0° C. The resulting mixture was stirred for 20 min at 0° C. then quenched by the addition of sodium thiosulfate (20 mL). The resulted mixture was extracted with ethyl acetate (3×5 mL), dried over anhydrous sodium sulfate. After filtration, the filtrate was concentrated under reduced pressure. The resulting residue was purified by RP-Flash with the following conditions: Column: C18 spherical Column, 20-35 μm, 100A, 80 g; Mobile Phase A: Water (5 mM aq. NH₄HCO₃), Mobile Phase B: MeCN; Flow rate: 60 mL/min; Gradient: 5% B to 40% B in 15 min, 40% B to 60% B in 10 min, 60% B to 60% B in 8 min, 60% B to 95% B in 10 min; Detector: UV 254 & 210 nm; RT: 33 min. The collected fractions were combined, concentrated and then lyophilized to give 2-(4-cyclopropyl-6-methoxy-pyrimidin-5-yl)-5-(2-trimethylsilylethoxymethyl)pyrrolo[3,2-d]pyrimidin-7-ol (60 mg, 145.09 μmol, 32% yield) as an off-white solid. MS: m/z=414.15 [M+H]⁺. ¹H NMR (400 MHz, Chloroform-d) δ 9.09 (s, 1H), 8.67 (s, 1H), 7.18 (s, 1H), 5.51 (s, 2H), 3.75 (s, 3H), 3.59-3.55 (m, 2H), 1.53-1.50 (m, 2H), 1.08-1.02 (m, 1H), 0.98-0.94 (m, 2H), 0.67-0.63 (m, 2H), 0.02 (s, 9H).

### 2-[[2-(4-cyclopropyl-6-methoxy-pyrimidin-5-yl)-7-[4-[1-methyl-4-(trifluoromethyl)imidazol-2-yl]phenoxy]pyrrolo[3,2-d]pyrimidin-5-yl]methoxy]ethyl-trimethyl-silane

To a stirred mixture of 2-(4-cyclopropyl-6-methoxy-pyrimidin-5-yl)-5-(2-trimethylsilylethoxymethyl)pyrrolo[3,2-d]pyrimidin-7-ol (40 mg, 97 μmol), 2-(4-bromophenyl)-1-methyl-4-(trifluoromethyl)imidazole (44 mg, 145, μmol) and cesium carbonate (32 mg, 97 μmol) in 1,4-dioxane (2 mL) was added rockphos palladacycle Gen. 3 (81 mg, 97 μmol) and RockPhos (45 mg, 97 μmol) under nitrogen atmosphere at 25° C. The resulting mixture was stirred at 100° C. for 16 hr. The reaction was cooled to room temperature, then diluted with water (10 mL), extracted with dichloromethane (3×5 mL). The combined organic layers were washed with brine (10 mL), dried over anhydrous sodium sulfate. After filtration, the filtrate was concentrated under reduced pressure. The residue was purified by RP-Flash with the following conditions: Column: C18 spherical Column, 20-35 μm, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 66% B in 8 min, 66% B to 66% B in 4 min, 66% B to 95% B in 8 min; Detector: UV 254 & 210 nm; RT: 16 min. The collected fractions were combined, concentrated and then lyophilized to give 2-[[2-(4-cyclopropyl-6-methoxy-pyrimidin-5-yl)-7-[4-[1-methyl-4-(trifluoromethyl)imidazol-2-yl]phenoxy]pyrrolo[3,2-d]pyrimidin-5-yl]methoxy]ethyl-trimethyl-silane (20 mg, 31.36 μmol, 32% yield) as an off-white solid. MS: m/z=638.25 [M+H]⁺.

### 2-(4-cyclopropyl-6-methoxy-pyrimidin-5-yl)-7-[4-[1-methyl-4-(trifluoromethyl)imidazol-2-yl]phenoxy]-5H-pyrrolo[3,2-d]pyrimidine

To a stirred solution of 2-[[2-(4-cyclopropyl-6-methoxy-pyrimidin-5-yl)-7-[4-[1-methyl-4-(trifluoromethyl)imidazol-2-yl]phenoxy]pyrrolo[3,2-d]pyrimidin-5-yl]methoxy]ethyl-trimethyl-silane (20 mg, 31.36 μmol) in DCM (1 mL) was added trifluoroacetic acid (1 mL) and then stirred for 2 hours at 25° C. The resulted mixture was concentrated under reduced pressure. Then ammonium hydroxide (28% in H₂O, 1 mL) and THF (1 mL) were added to the above residue. The resulting mixture was stirred for 30 min at 25° C. then concentrated under reduced pressure. The residue was purified by RP-Flash with the following conditions: Column: C18 spherical Column, 20-35 μm, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 65% B in 20 min, 65% B to 65% B in 4 min, 65% B to 95% B in 5 min; Detector: UV 254 & 210 nm; RT: 27 min. The collected fractions were combined, concentrated and then lyophilized to give 2-(4-cyclopropyl-6-methoxy-pyrimidin-5-yl)-7-[4-[1-methyl-4-(trifluoromethyl)imidazol-2-yl]phenoxy]-5H-pyrrolo[3,2-d]pyrimidine (4.9 mg, 9.66 μmol, 31% yield) as an off-white solid. MS: m/z=508.40 [M+H]⁺. ¹H NMR (400 MHz, Chloroform-d) δ 12.00 (br., 1H), 9.17 (s, 1H), 8.62 (s, 1H), 7.50 (d, J=8.4 Hz, 2H), 7.42 (s, 1H), 7.10 (d, J=8.4 Hz, 2H), 6.88 (s, 1H), 3.89 (s, 3H), 3.78 (s, 3H), 1.69-1.67 (m, 1H), 1.20-1.16 (m, 2H), 0.84-0.80 (m, 2H); ¹⁹F NMR (376 MHz, Chloroform-d) δ −62.30.

### Example 2. 7-[4-[1-methyl-4-(trifluoromethyl)imidazol-2-yl]phenyl]sulfanyl-2-[2-(trifluoromethyl)phenyl]-5H-pyrrolo[3,2-d]pyrimidine (Compound 84); 7-[4-[1-methyl-4-(trifluoromethyl)imidazol-2-yl]phenyl]sulfonyl-2-[2-(trifluoromethyl)phenyl]-5H-pyrrolo[3,2-d]pyrimidine (Compound 46) and 7-[4-[1-methyl-4-(trifluoromethyl)imidazol-2-yl]phenyl]sulfinyl-2-[2-(trifluoromethyl)phenyl]-5H-pyrrolo[3,2-d]pyrimidine (Compound 36)

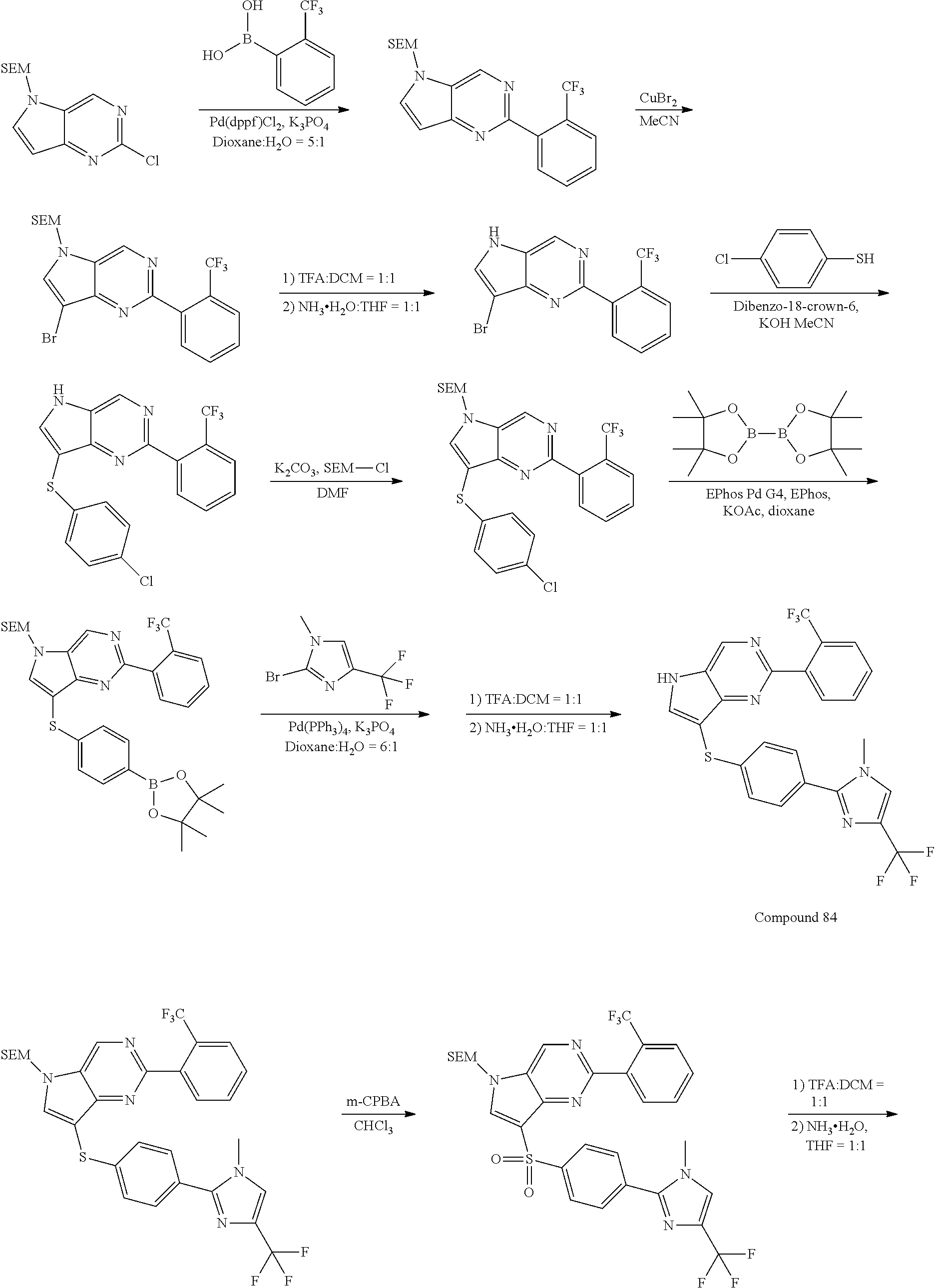

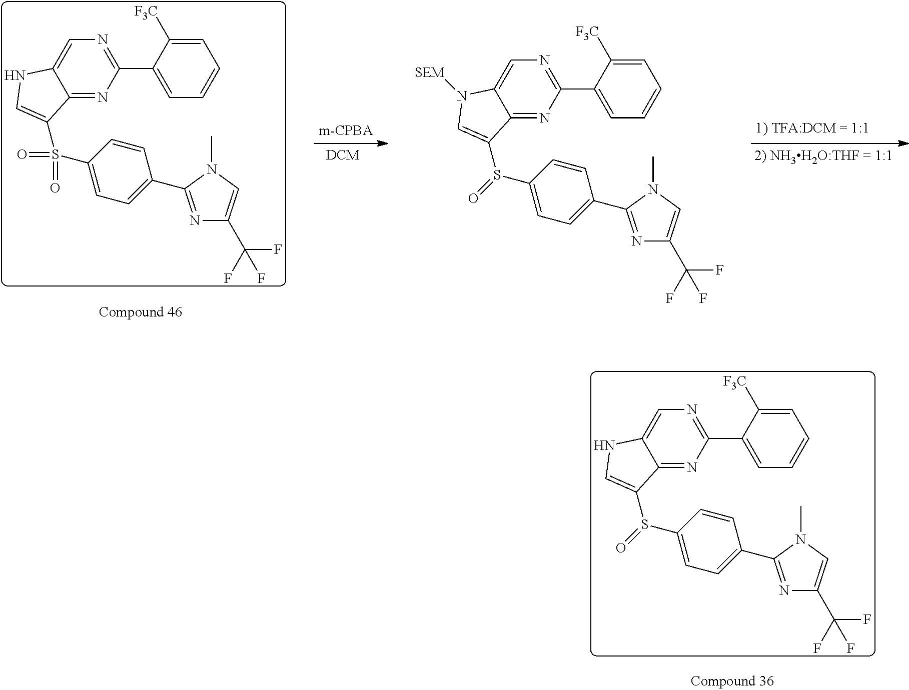

### trimethyl-[2-[[2-[2-(trifluoromethyl)phenyl]pyrrolo[3,2-d]pyrimidin-5-yl]methoxy]ethyl]silane

To a mixture of 2-[(2-chloropyrrolo[3,2d]pyrimidin-5-yl)methoxy]ethyl-trimethyl-silane (2 g, 7.05 mmol) and [2-(trifluoromethyl)phenyl]boronic acid (1.47 g, 7.75 mmol) in dioxane (30 mL) and water (6 mL) were added 1,1′-Bis(diphenylphosphino)ferrocene-palladium(II)dichloride dichloromethane complex (575 mg, 705 μmol) and potassium phosphate (2.99 g, 14.09 mmol) at 25° C. The resulting mixture was stirred at 100° C. for 16 hr. The resulting mixture was cooled to room temperature and the solid was filtered out. The filtrate was concentrated under reduced pressure. The residue was purified by silica gel column chromatography, eluted with 1%-50% ethyl acetate in petroleum ether to give trimethyl-[2-[[2-[2-(trifluoromethyl)phenyl]pyrrolo[3,2-d]pyrimidin-5-yl]methoxy]ethyl]silane (1.6 g, 4.07 mmol, 58% yield) as a yellow solid. MS: m/z=394.25 [M+H]⁺. ¹H NMR (400 MHz, Chloroform-d) δ9.08 (s, 1H), 7.82 (d, J=7.6 Hz, 1H), 7.77 (d, J=7.6 Hz, 1H), 7.66 (t, J=7.6 Hz, 1H), 7.63 (d, J=3.2 Hz, 1H), 7.56 (t, J=7.6 Hz, 1H), 6.79 (d, J=3.2 Hz, 1H), 5.61 (s, 2H), 3.56 (t, J=8.0 Hz, 2H), 0.95 (t, J=8.0 Hz, 2H), −0.01 (s, 9H).

### 2-[[7-bromo-2-[2-(trifluoromethyl)phenyl]pyrrolo[3,2-d]pyrimidin-5-yl]methoxy]ethyl-trimethyl-silane

To a solution of trimethyl-[2-[[2-[2-(trifluoromethyl)phenyl]pyrrolo[3,2-d]pyrimidin-5-yl]methoxy]ethyl]silane (2.5 g, 6.35 mmol) in acetonitrile (125 mL) was added cupric bromide (4.26 g, 19.06 mmol) at 0° C. The resulting mixture was stirred at 25° C. for 16 h. Then a solution of ammonia in methanol (12 mL, 7 N in methanol) was added to the reaction mixture and stirred for another 0.5 h. The resulting solution was diluted with water (300 mL), extracted with ethyl acetate (3×200 mL). The combined organic layers were dried over anhydrous sodium sulfate and filtered. The filtrate was concentrated under reduced pressure. The residue was purified by silica gel column chromatography, eluted with 1%-50% ethyl acetate in petroleum ether to give 2-[[7-bromo-2-[2-(trifluoromethyl)phenyl]pyrrolo[3,2-d]pyrimidin-5-yl]methoxy]ethyl-trimethyl-silane (2.4 g, 5.08 mmol, 80% yield) as a yellow solid. MS: m/z=471.95, 473.95 [M+H]⁺. ¹H NMR (400 MHz, Chloroform-d) δ 9.10 (s, 1H), 7.83 (d, J=8.0 Hz, 2H), 7.69 (s, 1H), 7.67 (t, J=7.6 Hz, 1H), 7.58 (t, J=7.6 Hz, 1H), 5.59 (s, 2H), 3.58 (t, J=8.0 Hz, 2H), 0.96 (t, J=8.0 Hz, 2H), 0.00 (s, 9H).

### 7-bromo-2-[2-(trifluoromethyl)phenyl]-5H-pyrrolo[3,2-d]pyrimidine

To a solution of 2-[[7-bromo-2-[2-(trifluoromethyl)phenyl]pyrrolo[3,2-d]pyrimidin-5-yl]methoxy]ethyl-trimethyl-silane (2.4 g, 5.08 mmol) in DCM (24 mL) was added trifluoroacetic acid (2.22 g, 19.47 mmol, 24 mL) at 25° C., and then stirred at this temperature for 1 h. The reaction solution was concentrated under reduced pressure. The residue was dissolved in THF (24 mL), then ammonium hydroxide (24 mL, 28% ammonia in water) was added at 0° C. The resulted mixture was stirred at 25° C. for 0.5 h and then concentrated under reduced pressure. The residue was purified by silica gel column chromatography, eluted with 1%-90% ethyl acetate in petroleum ether to give 7-bromo-2-[2-(trifluoromethyl)phenyl]-5H-pyrrolo[3,2-d]pyrimidine (1.6 g, 4.68 mmol, 92% yield) as a yellow solid. MS: m/z=342.10, 344.10 [M+H]⁺. ¹H NMR (400 MHz, Chloroform-d) δ 9.72 (s, 1H), 8.93 (s, 1H), 7.84-7.74 (m, 2H), 7.68-7.52 (m, 3H).

### 7-(4-chlorophenyl)sulfanyl-2-[2-(trifluoromethyl)phenyl]-5H-pyrrolo[3,2-d]pyrimidine

To a solution of 7-bromo-2-[2-(trifluoromethyl)phenyl]-5H-pyrrolo[3,2-d]pyrimidine (1.6 g, 4.68 mmol) and dibenzo-18-crown-6 (169 mg, 468 μmol) in acetonitrile (23 mL) was added potassium hydroxide (525 mg, 9.35 mmol) at 25° C. After stirred at 25° C. for 30 min, 4-chlorobenzenethiol (1.35 g, 9.35 mmol) was added to the mixture at 25° C. The resulted mixture was stirred at 60° C. for 16 h. The reaction was cooled to 25° C., and then quenched by the addition of saturated aqueous ammonium chloride (250 mL) at 0° C. The resulted mixture was extracted with ethyl acetate (3×200 mL). The combined organic layers were dried over anhydrous sodium sulfate. After filtration, the filtrate was concentrated under reduced pressure. The residue was purified by silica gel column chromatography, eluted with 1%-90% ethyl acetate in petroleum ether to give 7-(4-chlorophenyl)sulfanyl-2-[2-(trifluoromethyl)phenyl]-5H-pyrrolo[3,2-d]pyrimidine (840 mg, 2.07 mmol, 44% yield) as a yellow solid. MS: m/z=406.15, 408.15 [M+H]⁺. ¹H NMR (400 MHz, Chloroform-d) δ 10.14 (s, 1H), 8.93 (s, 1H), 7.81 (s, 1H), 7.74 (d, J=8.0 Hz, 1H), 7.67 (d, J=8.0 Hz, 1H), 7.58 (t, J=7.6 Hz, 1H), 7.50 (t, J=7.6 Hz, 1H), 7.13 (d, J=8.4 Hz, 2H), 7.06 (d, J=8.4 Hz, 2H).

### 2-[[7-(4-chlorophenyl)sulfanyl-2-[2-(trifluoromethyl)phenyl]pyrrolo[3,2-d]pyrimidin-5-yl]methoxy]ethyl-trimethyl-silane

To a stirred mixture of 7-(4-chlorophenyl)sulfanyl-2-[2-(trifluoromethyl)phenyl]-5H-pyrrolo[3,2-d]pyrimidine (840 mg, 2.07 mmol) and potassium carbonate (1.72 g, 12.42 mmol) in N,N-dimethylformamide (20 mL) was added 2-(trimethylsilyl)ethoxymethyl chloride (1.38 g, 8.28 mmol) dropwise with stirring at 0° C. The mixture was stirred at 25° C. for 16 hr. The reaction was diluted with ethyl acetate (300 mL), washed with brine (80 mL×3), dried over anhydrous sodium sulfate and filtered. The filtrate was concentrated under reduced pressure. The residue was purified by silica gel column chromatography, eluted with 0%-50% ethyl acetate in petroleum ether to give 2-[[7-(4-chlorophenyl)sulfanyl-2-[2-(trifluoromethyl)phenyl]pyrrolo[3,2-d]pyrimidin-5-yl]methoxy]ethyl-trimethyl-silane (880 mg, 1.64 mmol, 79% yield) as a light yellow solid. MS: m/z=536.10, 538.20 [M+H]⁺.

### trimethyl-[2-[[7-[4-(4,4,5,5-tetramethyl-1,3,2-dioxaborolan-2-yl)phenyl]sulfanyl-2-[2-(trifluoromethyl)phenyl]pyrrolo[3,2-d]pyrimidin-5-yl]methoxy]ethyl]silane

To a mixture of 2-[[7-(4-chlorophenyl)sulfanyl-2-[2-(trifluoromethyl)phenyl]pyrrolo[3,2-d]pyrimidin-5-yl]methoxy]ethyl-trimethyl-silane (660 mg, 1.23 mmol) and 4,4,5,5-tetramethyl-2-(4,4,5,5-tetramethyl-1,3,2-dioxaborolan-2-yl)-1,3,2-dioxaborolane (467 mg, 1.9 mmol) in dioxane (20 mL) were added EPhos Pd G4 (113 mg, 123 μmol) dicyclohexyl(3-isopropoxy-2′,4′,6′-triisopropyl-[1,1′-biphenyl]-2-yl)phosphane (65.8 mg, 123 μmol) and potassium acetate (362.5 mg, 3.7 mmol) at 25° C. The resulting mixture was stirred at 90° C. for 5 hr. The resulting mixture was cooled to room temperature and the solid was filtered out. The filtrate was concentrated under reduced pressure. The residue was purified by silica gel column chromatography, eluted with 1%-50% ethyl acetate in petroleum ether to give trimethyl-[2-[[7-[4-(4,4,5,5-tetramethyl-1,3,2-dioxaborolan-2-yl)phenyl]sulfanyl-2-[2-(trifluoromethyl)phenyl]pyrrolo[3,2-d]pyrimidin-5-yl]methoxy]ethyl]silane (573 mg, 913.00 μmol, 74% yield) as a yellow solid. MS: m/z=628.40 [M+H]⁺. ¹H NMR (400 MHz, Chloroform-d) δ 9.16 (s, 1H), 7.84 (s, 1H), 7.80-7.71 (m, 2H), 7.66-7.58 (m, 3H), 7.53 (t, J=7.6 Hz, 1H), 7.26-7.15 (m, 2H), 5.63 (s, 2H), 3.59 (t, J=8.0 Hz, 2H), 1.33 (s, 12H), 0.97 (t, J=8.0 Hz, 2H), −0.01 (s, 9H).

### trimethyl-[2-[[7-[4-[1-methyl-4-(trifluoromethyl)imidazol-2-yl]phenyl]sulfonyl-2-[2-(trifluoromethyl)phenyl]pyrrolo[3,2-S]pyrimidin-5-yl]methoxy]ethyl]silane

To a mixture of trimethyl-[2-[[7-[4-(4,4,5,5-tetramethyl-1,3,2-dioxaborolan-2-yl)phenyl]sulfanyl-2-[2-(trifluoromethyl)phenyl]pyrrolo[3,2-d]pyrimidin-5-yl]methoxy]ethyl]silane (570 mg, 908.22 μmol) and 2-bromo-1-methyl-4-(trifluoromethyl)imidazole (312 mg, 1.36 mmol) in dioxane (31 mL) and water (5 mL) were added tetrakis(triphenylphosphine)palladium (105 mg, 91 μmol) and potassium phosphate (386 mg, 1.8 mmol) at 25° C. The resulted mixture was stirred at 75° C. for 16 hr. The resulting mixture was cooled to room temperature and the solid was filtered out. The filtrate was concentrated under reduced pressure. The residue was purified by silica gel column chromatography, eluted with 1%-50% ethyl acetate in petroleum ether to give trimethyl-[2-[[7-[4-[1-methyl-4-(trifluoromethyl)imidazol-2-yl]phenyl]sulfanyl-2-[2-(trifluoromethyl)phenyl]pyrrolo[3,2-d]pyrimidin-5-yl]methoxy]ethyl]silane (560 mg, 861.90 μmol, 95% yield) as a yellow solid. MS: m/z=650.20 [M+H]⁺. ¹H NMR (400 MHz, Chloroform-d) δ 9.18 (s, 1H), 7.90 (s, 1H), 7.79-7.72 (m, 2H), 7.67-7.50 (m, 2H), 7.49-7.43 (m, 2H), 7.31-7.25 (m, 3H), 5.66 (s, 2H), 3.72 (s, 3H), 3.62 (t, J=8.0 Hz, 2H), 0.99 (t, J=8.0 Hz, 2H), 0.00 (s, 9H). ¹⁹F NMR (376 MHz, Chloroform-d) δ −57.23, −62.72.

### 7-[4-[1-methyl-4-(trifluoromethyl)imidazol-2-yl]phenyl]sulfanyl-2-[2-(trifluoromethyl)phenyl]-5H-pyrrolo[3,2-d]pyrimidine

To a solution of trimethyl-[2-[[7-[4-[1-methyl-4-(trifluoromethyl)imidazol-2-yl]phenyl]sulfanyl-2-[2-(trifluoromethyl)phenyl]pyrrolo[3,2-d]pyrimidin-5-yl]methoxy]ethyl]silane (150 mg, 230.87 μmol) in DCM (1.5 mL) was added trifluoroacetic acid (1.5 mL) at 25° C., and then stirred at this temperature for 1 h. The reaction mixture was concentrated under reduced pressure. The residue was dissolved in THF (1.5 mL) and then ammonium hydroxide (1.5 mL, 28% ammonia in water) was added at 0° C. The resulted mixture was stirred at 25° C. for 1 h and then concentrated under reduced pressure. The residue was purified RP-Flash with the following conditions: Column: C18 spherical Column, 20-35 um, 100A, 20 g; Mobile Phase A: Water (5 mM aq. NH₄HCO₃). Mobile Phase B: MeCN; Flow rate: 20 mL/min; Gradient: 5% B to 5% B in 5 min, 5% B to 55% B in 20 min, 55% B to 55% B in 4 min, 55% B to 95% B in 8 min; Detector: UV 254 & 210 nm; RT: 27 min. The collected fractions were combined, concentrated and then lyophilized overnight to give 7-[4-[1-methyl-4-(trifluoromethyl)imidazol-2-yl]phenyl]sulfanyl-2-[2-(trifluoromethyl)phenyl]-5H-pyrrolo[3,2-d]pyrimidine (80 mg, 154.00 μmol, 67% yield) as an off-white solid. MS: m/z=520.15 [M+H]⁺. ¹H NMR (400 MHz, Methanol-4) S 9.06 (s, 1H), 8.24 (s, 1H), 7.81-7.69 (m, 2H), 7.66-7.62 (m, 3H), 7.47 (d, J=8.4 Hz, 2H), 7.25 (d, J=8.4 Hz, 2H), 3.73 (s, 3H). ¹⁹F NMR (376 MHz, Methanol-d₄) δ −58.755, −63.96.

### trimethyl-[2-[[7-[4-[1-methyl-4-(trifluoromethyl)imidazol-2-yl]phenyl]sulfonyl-2-[2-(trifluoromethyl)phenyl]pyrrolo[3,2-d]pyrimidin-5-yl]methoxy]ethyl]silane

To a solution of trimethyl-[2-[[7-[4-[1-methyl-4-(trifluoromethyl)imidazol-2-yl]phenyl]sulfanyl-2-[2-(trifluoromethyl)phenyl]pyrrolo[3,2-d]pyrimidin-5-yl]methoxy]ethyl]silane (20 mg, 30.78 μmol) in chloroform (1 mL) was added 3-chlorobenzenecarboperoxoic acid (17.7 mg, 71.7 μmol, 70% purity) at 0° C. The resulting mixture was stirred at 25° C. for 2 h. The residue was diluted with ethyl acetate (10 mL) and then added to cold saturated aqueous sodium thiosulfate (50 mL) dropwise with stirring at 0° C. The resulted mixture was extracted with ethyl acetate (3×15 mL), the combined organic layers were washed with brine (20 mL), dried over anhydrous sodium sulfate and filtered. The filtrate was concentrated under reducing pressure to give trimethyl-[2-[[7-[4-[1-methyl-4-(trifluoromethyl)imidazol-2-yl]phenyl]sulfonyl-2-[2-(trifluoromethyl)phenyl]pyrrolo[3,2-d]pyrimidin-5-yl]methoxy]ethyl]silane (20 mg, 29.34 μmol, 95% yield) as a yellow solid which was used for next step directly without further purification. MS: m/z=682.20 [M+H]⁺.

### 7-[4-[1-methyl-4-(trifluoromethyl)imidazol-2-yl]phenyl]sulfonyl-2-[2-(trifluoromethyl)phenyl]-5H-pyrrolo[3,2-d]pyrimidine

To a stirred solution of trimethyl-[2-[[7-[4-[1-methyl-4-(trifluoromethyl)imidazol-2-yl]phenyl]sulfonyl-2-[2-(trifluoromethyl)phenyl]pyrrolo[3,2-d]pyrimidin-5-yl]methoxy]ethyl]silane (20 mg, 29.34 μmol) in DCM (2 mL) was added trifluoroacetic acid (2 mL) and then stirred at 25° C. for 2 hours. The resulting mixture was concentrated under reduced pressure. The residue was dissolved in THF (2 mL) and then ammonium hydroxide (2 mL, 28% ammonia in water) was added. After stirred at 25° C. for 30 min, the reaction mixture was concentrated under reduced pressure. The residue was purified by RP-Flash with the following conditions: Column: Spherical C18, 20-40 μm, 40 g; Mobile Phase A: Water (5 mM aq. NH₄HCO₃), Mobile Phase B: MeCN; Flow rate: 40 mL/min; Gradient (B %): 2% B to 2% B in 5 min, 2% B to 54% B in 14 min, 54% B to 54% B in 3 min, 54% B to 95% B in 10 min, Detector: UV 254 & 210 nm; RT: 20 min. The collected fractions were combined, concentrated and then lyophilized overnight to give 7-[4-[1-methyl-4-(trifluoromethyl)imidazol-2-yl]phenyl]sulfonyl-2-[2-(trifluoromethyl)phenyl]-5H-pyrrolo[3,2-d]pyrimidine (6.2 mg, 11.24 μmol, 38% yield) as an off-white solid. MS: m/z=552.05 [M+H]⁺. ¹H NMR (400 MHz, Methanol-d₄) δ 9.05 (s, 1H), 8.56 (s, 1H), 8.37 (d, J=8.4 Hz, 2H), 7.87-7.84 (m, 3H), 7.80-7.67 (m, 4H), 3.79 (s, 3H). ¹⁹F NMR (376 MHz, Methanol-d₄) δ+−58.465, −64.04.

### trimethyl-[2-[[7-[4-[1-methyl-4-(trifluoromethyl)imidazol-2-yl]phenyl]sulfanyl-2-[2-(trifluoromethyl)phenyl]pyrrolo[3,2-d]pyrimidin-5-yl]methoxy]ethyl]silane

To a stirred solution of trimethyl-[2-[[7-[4-[1-methyl-4-(trifluoromethyl)imidazol-2-yl]phenyl]sulfanyl-2-[2-(trifluoromethyl)phenyl]pyrrolo[3,2-d]pyrimidin-5-yl]methoxy]ethyl]silane (110 mg, 169.3 μmol) in DCM (1 mL) was added 3-chlorobenzenecarboperoxoic acid (29 mg, 118.5 μmol, 70% purity) at 0° C. under nitrogen atmosphere. After stirred at 0° C. for 1 hr, the reaction was quenched by saturated aqueous sodium thiosulfate (20 mL) at 0° C. The resulting mixture was extracted with ethyl acetate (3×20 mL). The combined organic layers were dried over anhydrous sodium sulfate. After filtration, the combined organic layers were concentrated under reduced pressure to give trimethyl-[2-[[7-[4-[1-methyl-4-(trifluoromethyl)imidazol-2-yl]phenyl]sulfinyl-2-[2-(trifluoromethyl)phenyl]pyrrolo[3,2-d]pyrimidin-5-yl]methoxy]ethyl]silane (90 mg, 135.19 μmol, 80% yield) as an off-white solid which was used for next step directly without further purification. MS: m/z=666.35 [M+H]⁺.

### 7-[4-[1-methyl-4-(trifluoromethyl)imidazol-2-yl]phenyl]sulfinyl-2-[2-(trifluoromethyl)phenyl]-5H-pyrrolo[3,2-d]pyrimidine

To a stirred solution of trimethyl-[2-[[7-[4-[1-methyl-4-(trifluoromethyl)imidazol-2-yl]phenyl]sulfinyl-2-[2-(trifluoromethyl)phenyl]pyrrolo[3,2-d]pyrimidin-5-yl]methoxy]ethyl]silane (90 mg, 135.19 μmol) in DCM (2 mL) was added trifluoroacetic acid (2 mL) and then stirred for 2 hours at 25° C. The resulted mixture was concentrated under reduced pressure. The residue was dissolved in THF (2 mL) and then then ammonium hydroxide (2 mL, 28%) was added. The resulted mixture was stirred at 25° C. for 30 min and then concentrated under reduced pressure. The residue was purified by RP-Flash with the following conditions: (Column: Spherical C18, 20-40 μm, 40 g; Mobile Phase A: Water (5 mM aq. NH₄HCO₃), Mobile Phase B: MeCN; Flow rate: 40 mL/min; Gradient (B %): 2% B to 2% B in 5 min, 2% B to 62% B in 15 min, 62% B to 62% B in 3 min, 62% B to 95% B in 10 min, Detector: UV 254 & 210 nm; RT: 21 min. The collected fractions were combined, concentrated and then lyophilized overnight to give 7-[4-[1-methyl-4-(trifluoromethyl)imidazol-2-yl]phenyl]sulfinyl-2-[2-(trifluoromethyl)phenyl]-5H-pyrrolo[3,2-d]pyrimidine (40 mg, 74.70 μmol, 55% yield) as an off-white solid. MS: m/z=536.20 [M+H]⁺. ¹H NMR (400 MHz, Methanol-d₄) δ 9.05 (s, 1H), 8.32 (s, 1H), 8.01 (d, J=8.4 Hz, 2H), 7.86-7.82 (m, 3H), 7.74-7.61 (m, 4H), 3.78 (s, 3H). ¹⁹F NMR (376 MHz, Methanol-d₄) δ −58.562, −63.99.

### Example 3. Synthesis of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (Compound 5)

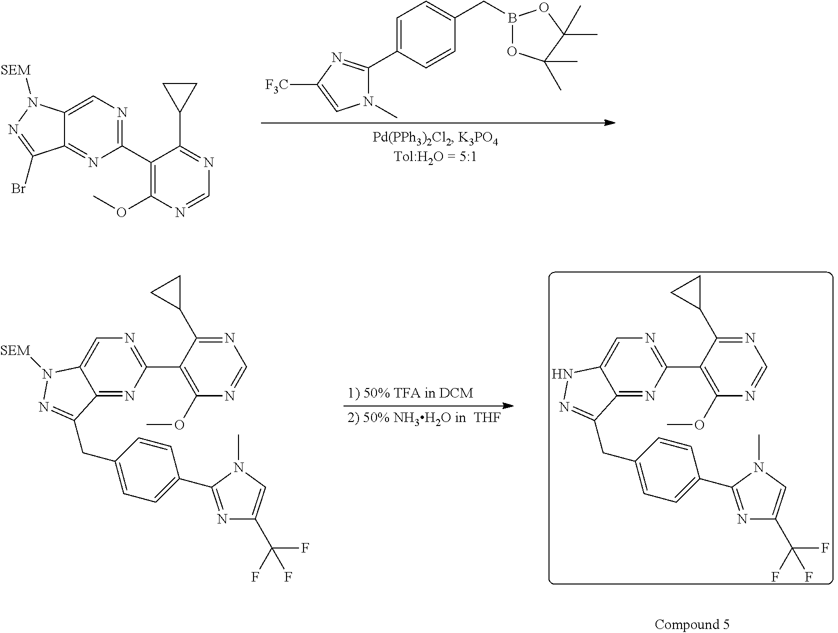 [SMILES: COc1ncnc(C2CC2)c1-c1ncc2c(n1)c(Br)nn2COCC[Si](C)(C)C ; Cn1cc(C(F)(F)F)nc1-c1ccc(CB2OC(C)(C)C(C)(C)O2)cc1 ; COc1ncnc(C2CC2)c1-c1ncc2c(n1)c(Cc1ccc(-c3nc(C(F)(F)F)cn3C)cc1)nn2COCC[Si](C)(C)C ; COc1ncnc(C2CC2)c1-c1ncc2[nH]nc(Cc3ccc(-c4nc(C(F)(F)F)cn4C)cc3)c2n1]

### 2-[[5-(4-cyclopropyl-6-methoxy-pyrimidin-S-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane

To a solution of 2-[[3-bromo-5-(4-cyclopropyl-6-methoxy-pyrimidin-5-yl)pyrazolo[4,3-d]pyrimidin-1-yl]methoxy]ethyl-trimethyl-silane (70 mg, 146.62 μmol) and 1-methyl-2-[4-[(4,4,5,5-tetramethyl-1,3,2-dioxaborolan-2-yl)methyl]phenyl]-4-(trifluoromethyl)imidazole (80.5 mg, 219.9 μmol) in toluene and water (v/v, 5:1, 2.4 mL) were added bis(triphenylphosphine)palladium (II) chloride (10.3 mg, 14.7 μmol) and potassium acetate (43 mg, 440 μmol, 27.5 μL) at 25° C. under N₂ atmosphere. Then the mixture was heated to 90° C. and stirred for 16 h under N₂ atmosphere. The mixture was allowed to cool down to 25° C. Then the mixture was diluted with brine (20 mL) and extracted with ethyl acetate (3×20 mL), dried over anhydrous sodium sulfate. After filtration, the filtrate was concentrated under reduced pressure. The residue was purified by silica gel column chromatography, eluted with 60˜70% ethyl acetate in petroleum ether to give 2-[[5-(4-cyclopropyl-6-methoxy-pyrimidin-5-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane (35 mg, 54.97 umol, 38% yield) as a yellow solid. MS: m/z=637.30 [M+H]⁺.

### 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine

To a solution of 2-[[5-(4-cyclopropyl-6-methoxy-pyrimidin-5-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane (35 mg, 54.97 μmol) in dichloromethane (2 mL) was added trifluoroacetic acid (3.11 g, 27.26 mmol, 2.10 mL) at 0° C. under N₂ atmosphere. The reaction mixture was stirred at 25° C. for 2 h and then concentrated under reduced pressure. The mixture was dissolved in tetrahydrofuran (3 mL) and 28% aq. ammonium hydroxide (1.89 g, 53.93 mmol, 2.10 mL). After stirred at 25° C. for 30 minutes, the reaction solution was concentrated under reduced pressure. The residue was purified by silica gel column chromatography, eluted with 80˜90% ethyl acetate in petroleum ether to give a crude product. The crude product was purified by RP-Flash with the following conditions: Column: C18 spherical Column, 20-35 um, 100A, 80 g; Mobile Phase A: Water (5 mM aq. NH₄HCO₃), Mobile Phase B: MeCN; Flow rate: 50 mL/min; Gradient: 5% B to 5% B in 5 min, 5% B to 35% B in 10 min. 35% B to 35% B in 3 min. 35% B to 95% B in 15 min; Detector: UV 254 & 210 nm; RT: 17 min. The collected fractions were combined, concentrated and then lyophilized overnight to 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (10.7 mg, 21.13 μmol, 38% yield) as a white solid. MS: m/z=507.25 [M+H]⁺. ¹H NMR (400 MHz, Chloroform-d) δ 12.90 (s, 1H), 9.20 (s, 1H), 8.68 (s, 1H), 7.67 (d, J=8.0 Hz, 2H), 7.60 (d, J=8.4 Hz, 2H), 7.40 (d, J=1.2 Hz, 1H), 4.55 (s, 2H), 3.92 (s, 3H), 3.82 (s, 3H), 1.67-1.63 (m, 1H), 1.25-1.19 (m, 2H), 0.86-081 (m, 2H); ¹⁹F NMR (376 MHz, Chloroform-d) δ −62.26.

### Example 4. Synthesis of 5-(4-cyclopropyl-6-methoxy-pyrimidin-5-yl)-2-methyl-3-[[4-[1-methyl-4-(trifluoromethyl)imidazol-2-yl]phenyl]methyl]pyrazolo[4,3-d]pyrimidine (Compound 24) & 5-(4-cyclopropyl-6-methoxy-pyrimidin-5-yl)-1-methyl-3-[[4-[1-methyl-4-(trifluoromethyl)imidazol-2-yl]phenyl]methyl]pyrazolo[4,3-d]pyrimidine (Compound 26)

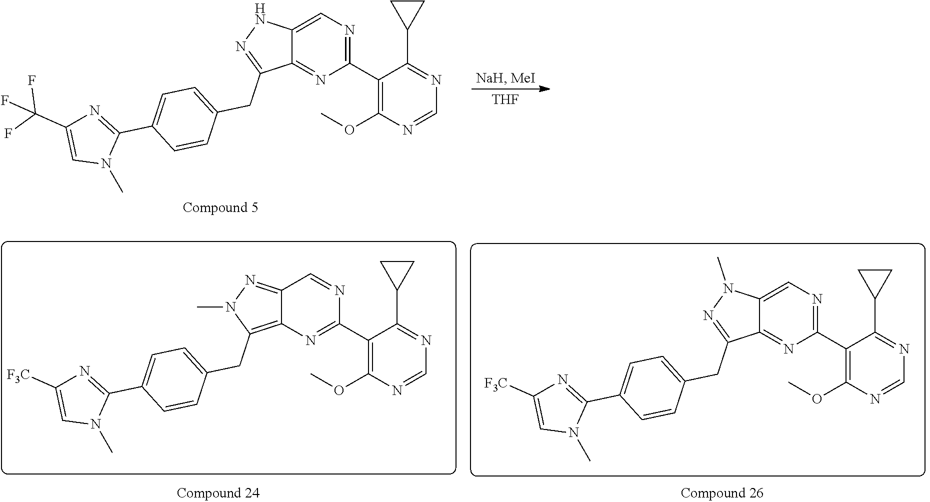 [SMILES: COc1ncnc(C2CC2)c1-c1ncc2[nH]nc(Cc3ccc(-c4nc(C(F)(F)F)cn4C)cc3)c2n1 ; COc1ncnc(C2CC2)c1-c1ncc2nn(C)c(Cc3ccc(-c4nc(C(F)(F)F)cn4C)cc3)c2n1 ; COc1ncnc(C2CC2)c1-c1ncc2c(n1)c(Cc1ccc(-c3nc(C(F)(F)F)cn3C)cc1)nn2C ; "THF"]

### 5-(4-cyclopropyl-6-methoxy-pyrimidin-5-yl)-2-methyl-3-[[4-[1-methyl-4-(trifluoromethyl)imidazol-2-yl]phenyl]methyl]pyrazolo[4,3-d]pyrimidine & 5-(4-cyclopropyl-6-methoxy-pyrimidin-5-yl)-1-methyl-3-[[4-[1-methyl-4-(trifluoromethyl)imidazol-2-yl]phenyl]methyl]pyrazolo[4,3-d]pyrimidine

To a stirring mixture of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (50 mg, 98.72 μmol) in THF (1 mL) was added sodium hydride (8 mg, 197 μmol, 60% purity) under nitrogen atmosphere at 0° C. The resulting mixture was stirred for 1 hr at 0° C. To the above mixture was added methyl iodide (21 mg, 148 μmol, 9.2 μL) under nitrogen atmosphere at 25° C. The resulting mixture was stirred at 25° C. for 2 hr then quenched by the addition of saturated aqueous ammonium chloride (30 mL). The resulting mixture was extracted with ethyl acetate (3×10 mL). The combined organic layers were washed with brine (3×10 mL), dried over anhydrous sodium sulfate. After filtration, the filtrate was concentrated under reduced pressure. The residue was purified by Prep-TLC, eluted with 8% methanol in dichloromethane to give 38 mg crude product. The crude product was purified by RP-Flash with the following conditions: Column: C18 spherical Column, 20-35 μm, 100A, 40 g; Mobile Phase A: Water (5 mM aq. NH₄HCO₃), Mobile Phase B: MeCN. Flow rate: 60 mL/min; Gradient: 5% B to 5% B in 5 min, 5% B to 56% B in 10 min, 56% B to 56% B in 5 min. 56% B to 95% B in 5 min; Detector: UV 254 & 210 nm; RT1: 17 min; RT2: 19 min. The first product (RT1: 17 min) peak were combined, concentrated and then lyophilized to give 5-(4-cyclopropyl-6-methoxy-pyrimidin-5-yl)-2-methyl-3-[[4-[-methyl-4-(trifluoromethyl)imidazol-2-yl]phenyl]methyl]pyrazolo[4,3-d]pyrimidine (10.2 mg, 19.60 μmol, 20% yield) as an off-white solid. MS: m/z=521.35 [M+H]⁺. ¹H NMR (400 MHz, Chloroform-d) δ 9.54 (s, 1H), 8.68 (s, 1H), 7.58 (d, J=8.0 Hz, 2H), 7.36-7.29 (m, 3H), 4.63 (s, 2H), 4.15 (s, 3H), 3.96 (s, 3H), 3.75 (s, 3H), 1.82-1.70 (m, 1H), 1.30-1.22 (m, 2H), 0.97-0.86 (m, 2H); ¹⁹F NMR (376 MHz, Chloroform-d) δ −62.77.

The second product (RT2: 19 min) peak was combined and concentrated and then lyophilized to give 5-(4-cyclopropyl-6-methoxy-pyrimidin-5-yl)-1-methyl-3-[[4-[1-methyl-4-(trifluoromethyl)imidazol-2-yl]phenyl]methyl]pyrazolo[4,3-d]pyrimidine (10.5 mg, 20.17 μmol, 20% yield) as an off-white solid. MS: m/z=521.40 [M+H]⁺. ¹H NMR (400 MHz, Chloroform-d) δ 9.22 (s, 1H), 8.70 (s, 1H), 7.60-7.51 (m, 4H), 7.30 (s, 1H), 4.52 (s, 2H), 4.21 (s, 3H), 3.94 (s, 3H), 3.74 (s, 3H), 1.72-1.61 (m, 1H), 1.30-1.21 (m, 2H), 0.93-0.83 (m, 2H); ¹⁹F NMR (376 MHz, Chloroform-d) δ −62.74.

### Example 5. Synthesis of 5-(2-isopropylphenyl)-3-[[4-[1-methyl-4-(trifluoromethyl)imidazol-2-yl]phenyl]methyl]-1H-pyrazolo[4,3-d]pyrimidine (Compound 27)

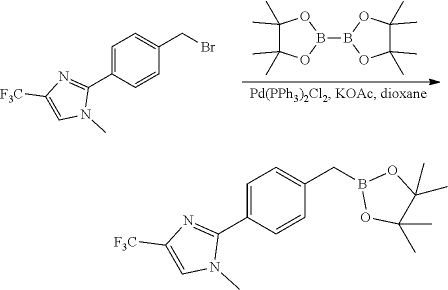

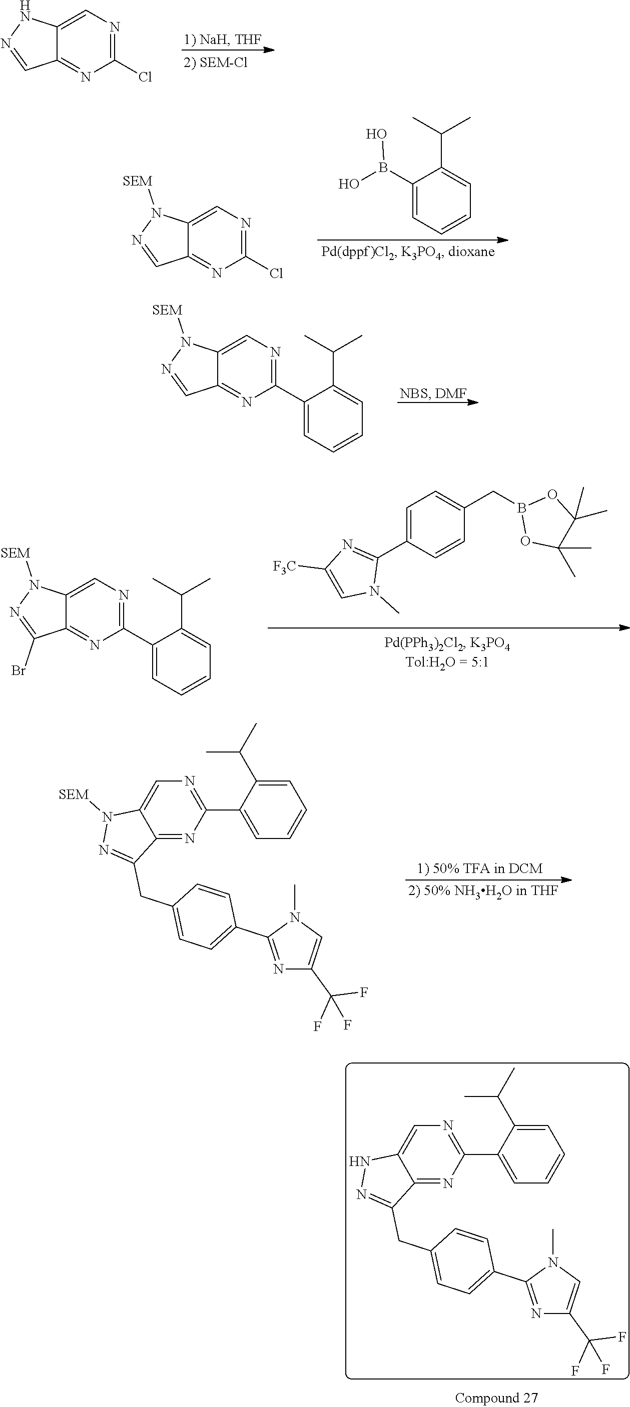

### 1-methyl-2-[4-[(4,4,5,5-tetramethyl-1,3,2-dioxaborolan-2-yl)methyl]phenyl]-4-(trifluoromethyl)imidazole

To a solution of 2-[4-(bromomethyl)phenyl]-1-methyl-4-(trifluoromethyl)imidazole (500 mg, 1.57 mmol) and 4,4,5,5-tetramethyl-2-(4,4,5,5-tetramethyl-1,3,2-dioxaborolan-2-yl)-1,3,2-dioxaborolane (597 mg, 2.35 mmol) in Dioxane (5 mL) were added bis(triphenylphosphine)palladium(II) chloride (110 mg, 157 μmol) and potassium acetate (461 mg, 4.7 mmol, 294 μL) at 25° C. under N₂ atmosphere. The resulting mixture was heated to 80° C., and stirred for 16 h under N₂ atmosphere then was cooled down to 25° C. Then the mixture was diluted with EA (200 mL) and washed with brine (200 mL×4), dried over anhydrous sodium sulfate and concentrated under reduced pressure. The residue was purified by chromatography on silica gel elute with 40%-50% EtOAc in PE to afford 1-methyl-2-[4-[(4,4,5,5-tetramethyl-1,3,2-dioxaborolan-2-yl)methyl]phenyl]-4-(trifluoromethyl)imidazole (518 mg, 1.41 mmol, 90% yield) as a white solid. MS: m/z=367.17 [M+H]⁺. ¹H NMR (400 MHz, Chloroform-d) δ 7.56-7.47 (m, 2H), 7.34-7.27 (m, 3H), 3.78 (s, 3H), 2.36 (s, 2H), 1.29 (d, J=4.0 Hz, 12H).

### 2-[(5-chloropyrazolo[4,3-d]pyrimidin-1-yl)methoxy]ethyl-trimethyl-silane

To a solution of 5-chloro-1H-pyrazolo[4,3-d]pyrimidine (500 mg, 3.24 mmol) in THF (5 mL) was added sodium hydride (60% dispersion in mineral oil, 97 mg, 4.0 mmol) at 0° C. under N₂ atmosphere, and the reaction mixture was stirred at this temperature for 45 min. Then 2-(chloromethoxy)ethyl-trimethyl-silane (674 mg, 4.04 mmol, 716 μL) was added to the reaction mixture and stirred at 25° C. for 2 h. The reaction was quenched with saturated aqueous ammonium chloride (200 mL), and then the mixture was extracted with EtOAc (200×3 mL).

The combined organic extracts were washed with brine (100 mL), dried over anhydrous Na₂SO₄, and concentrated under vacuum. The residue was further purified by chromatography on silica gel elute with 40%-45% EtOAc in PE to give 2-[(5-chloropyrazolo[4,3-d]pyrimidin-1-yl)methoxy]ethyl-trimethyl-silane (700 mg, 2.46 mmol, 76% yield) as a light-yellow oil. MS: m/z=285.08 [M+H]⁺. ¹H NMR (400 MHz, Chloroform-d) δ 9.14 (s, 1H), 8.18 (s, 1H), 5.81 (s, 2H), 3.59-3.50 (m, 2H), 0.99-0.84 (m, 2H), −0.06 (s, 9H).

### 2-[[5-(2-isopropylphenyl)pyrazolo[4,3-d]pyrimidin-1-yl]methoxy]ethyl-trimethyl-silane

To a solution of 2-[(5-chloropyrazolo[4,3-d]pyrimidin-1-yl)methoxy]ethyl-trimethyl-silane (700 mg, 2.46 mmol) and (2-isopropylphenyl)boronic acid (403 mg, 2.46 mmol) in 1,4-dioxane (7 mL) were added 1,1′-Bis(diphenylphosphino)ferrocene-palladium (II) dichloromethane complex (2.01 g, 2.46 mmol) and potassium phosphate tribasic anhydrous (522 mg, 2.5 mmol) at 25° C. under N₂ atmosphere. After the resulting mixture was stirred at 100° C. for 8 h under N₂ atmosphere, it was cooled down to 25° C., and then diluted with EA (200 mL) and washed with brine (4×200 mL), dried over anhydrous sodium sulfate. The organic phase was concentrated under reduced pressure. The residue was purified by chromatography on silica gel elute with 40%-50% EtOAc in PE to afford 2-[[5-(2-isopropylphenyl)pyrazolo[4,3-d]pyrimidin-1-yl]methoxy]ethyl-trimethyl-silane (400 mg, 1.09 mmol, 44% yield) as a yellow oil. MS: m/z=369.20 [M+H]⁺. ¹H NMR (400 MHz, Chloroform-d) δ 9.59 (s, 1H), 8.46 (s, 1H), 7.67-7.60 (m, 1H), 7.52-7.41 (m, 2H), 7.33-7.31 (m, 1H), 5.85 (s, 2H), 3.76-3.68 (m, 2H), 3.63-3.46 (m, 1H), 1.25 (d, J=6.8 Hz, 6H), 1.26-1.25 (m, 2H), 0.00 (s, 9H).

### 2-[[3-bromo-5-(2-isopropylphenyl)pyrazolo[4,3-d]pyrimidin-1-yl]methoxy]ethyl-trimethyl-silane

To a solution of 2-[[5-(2-isopropylphenyl)pyrazolo[4,3-d]pyrimidin-1-yl]methoxy]ethyl-trimethyl-silane (400 mg, 1.09 mmol) in DMF (4 mL) was added 1-bromopyrrolidine-2,5-dione (464 mg, 2.60 mmol, 221 μL) at 25° C. under N₂ atmosphere. The reaction mixture was stirred at 25° C. for 8 h. Then the reaction was quenched with brine (50 mL), extracted with EA (3×100 mL). The combine organic was dried over anhydrous sodium sulfate. After filtration, the filtrate was concentrated under vacuum and the residue was purified by chromatography on silica gel, eluted with 30%-40% EtOAc in PE to afford 2-[[3-bromo-5-(2-isopropylphenyl)pyrazolo[4,3-d]pyrimidin-1-yl]methoxy]ethyl-trimethyl-silane (400 mg, 893.97 μmol, 82% yield) as a yellow oil. MS: m/z=447.12, 449.12 [M+H]⁺. ¹H NMR (400 MHz, Chloroform-d) δ 9.56 (s, 1H), 7.74-7.70 (m, 1H), 7.53-7.41 (m, 2H), 7.40-7.30 (m, 1H), 5.93 (s, 2H), 3.80-3.71 (m, 2H), 3.56-3.50 (m, 1H), 1.29 (d, J=6.8 Hz, 6H), 1.04-0.95 (m, 2H), 0.00 (s, 9H).

### 2-[[5-(2-isopropylphenyl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane

To a solution of 2-[[3-bromo-5-(2-isopropylphenyl)pyrazolo[4,3-d]pyrimidin-1-yl]methoxy]ethyl-trimethyl-silane (280 mg, 625.78 umol) and 1-methyl-2-[[4-[(4,4,5,5-tetramethyl-1,3,2-dioxaborolan-2-yl)methyl]phenyl]-4-(trifluoromethyl)imidazole (344 mg, 937 μmol) in toluene (8 mL) and 20 (1.6 mL) were added bis(triphenylphosphine)palladium(II) chloride (44 mg, 62.6 μmol) and potassium phosphate tribasic anhydrous (266 mg, 1.25 mmol) at 25° C. under N₂ atmosphere. After the resulting mixture was heated to 90° C., and stirred for 16 h under N₂ atmosphere, it was cooled down to 25° C., and then diluted with EA (200 mL) and washed with brine (4×200 mL), dried over anhydrous sodium sulfate. The organic phase was concentrated under reduced pressure. The residue was purified by chromatography on silica gel elute with 50%-60% EtOAc in PE to afford 2-[[5-(2-isopropylphenyl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane (220 mg, 362.58 μmol, 58% yield) as a yellow solid. MS: m/z=607.27 [M+H]⁺. ¹H NMR (400 MHz, Chloroform-d) δ 9.55 (s, 1H), 7.75-7.72 (m, 1H), 7.57 (d, J=8.0 Hz, 2H), 7.54-7.37 (m, 4H), 7.37-7.26 (m, 2H), 5.72 (s, 2H), 4.69 (s, 2H), 3.74 (s, 3H), 3.65-3.55 (m, 3H), 1.29 (d, J=6.8 Hz, 6H), 0.97-0.87 (m, 2H), 0.00 (s, 9H).

### 5-(2-isopropylphenyl)-3-[[4-[1-methyl-4-(trifluoromethyl)imidazol-2-yl]phenyl]methyl]-1H-pyrazolo[4,3-d]pyrimidine

To a solution of 2-[[5-(2-isopropylphenyl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane (200 mg, 329.62 μmol) in DCM (3 mL) was added trifluoroacetic acid (7.4 g, 64.9 mmol, 5 mL) at 0° C. under N₂ atmosphere. After the reaction mixture was stirred at 25° C. for 2 h, it was concentrated under vacuum. The residue was added 28% aq. ammonium hydroxide (4.50 g 128.40 mmol, 5 mL) and THF (5 mL) and the resulting mixture was stirred at 25° C. for 30 minutes. The reaction solution was concentrated under vacuum and the residue was purified by chromatography on silica gel eluted with 80%-90% EtOAc in PE to give crude product. The crude product was further purified by RP-Flash chromatography with the following conditions: Column: C18 gel column (80 g), 20-35 μm, 19×150 mm; mobile phase: acetonitrile and 5 mM aq. ammonium hydrogen carbonate; Gradient: 55% hold 10 min, up to 70% within 20 min; Flow rate: 50 mL/min; Detector: UV 254 & 210 nm; RT: 18 min. The fractions of first eluting peak (RT: 18 min) were collected and concentrated under reduced pressure and then lyophilized overnight to afford 5-(2-isopropylphenyl)-3-[[4-[1-methyl-4-(trifluoromethyl)imidazol-2-yl]phenyl]methyl]-1H-pyrazolo[4,3-d]pyrimidine (97.5 mg, 204.62 μmol, 62% yield) as a white solid. MS: m/z=477.19 [M+H]⁺. ¹H NMR (400 MHz, Chloroform-d) δ 9.18 (s, 1H), 7.68-7.61 (m, 3H), 7.57 (d, J=8.0 Hz, 2H), 7.52-7.40 (m, 2H), 7.36 (s, 1H), 7.35-7.27 (m, 1H), 4.54 (s, 2H), 3.79 (s, 3H), 3.50-3.44 (m, 1H), 1.24 (d, J=6.8 Hz, 6H). ¹⁹F NMR (376 MHz, Chloroform-d) δ −62.38.

### Example 6. 5-(4-cyclopropyl-6-methoxy-pyrimidin-5-yl)-3-[[4-[1-isopropyl-4-(trifluoromethyl)imidazol-2-yl]phenyl]methyl]-1H-pyrazolo[4,3-d]pyrimidine (Compound 16)

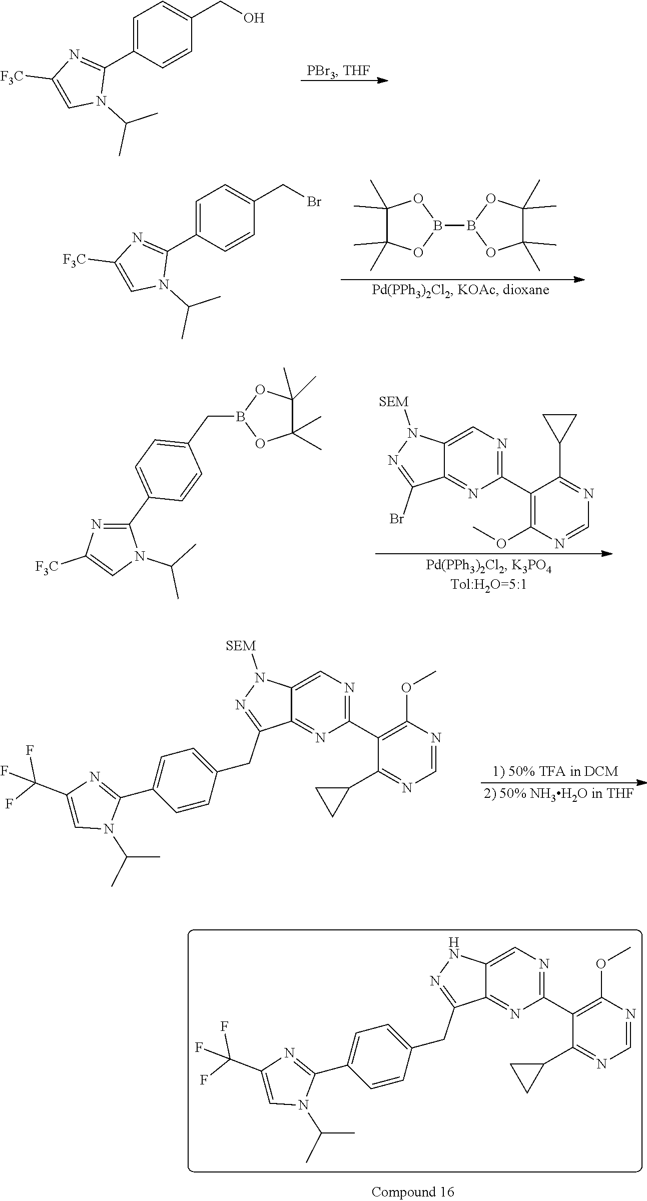

### 2-[4-(bromomethyl)phenyl]-1-isopropyl-4-(trifluoromethyl)imidazole

To a solution of [4-[1-isopropyl-4-(trifluoromethyl)imidazol-2-yl]phenyl]methanol (400 mg, 1.41 mmol) in THF (8 mL) was added phosphorus(III) tribromide (1.90 g, 7.0 mmol, 661 μL) at 0° C. Then the mixture was stirred at 25° C. for 2.5 h. The resulted solution was quenched by saturated aqueous sodium bicarbonate (200 mL), then was extracted by ethyl acetate (150 mL×3). The organic layer was dried over anhydrous sodium sulfate. After filtration, the filtrate was concentrated under reduced pressure. The residue was purified by silica gel column chromatography, eluted with 40˜45% ethyl acetate in petroleum ether to afford 2-[4-(bromomethyl)phenyl]-1-isopropyl-4-(trifluoromethyl)imidazole (380 mg, 1.09 mmol, 78% yield) as an off-white solid. MS: m/z=346.90, 348.90 [M+H]⁺. ¹H NMR (400 MHz, Chloroform-d) δ 7.59-7.49 (m, 4H), 7.45 (s, 1H), 4.67-4.56 (m, 1H), 4.55 (s, 2H), 1.48 (d, J=6.8 Hz, 6H).

### 1-isopropyl-2-[4-[(4,4,5,5-tetramethyl-1,3,2-dioxaborolan-2-yl)methyl]phenyl]-4-(trifluoromethyl)imidazole

To a solution of 2-[4-(bromomethyl)phenyl]-1-isopropyl-4-(trifluoromethyl)imidazole (380 mg, 1.09 mmol) and 4,4,5,5-tetramethyl-2-(4,4,5,5-tetramethyl-1,3,2-dioxaborolan-2-yl)-1,3,2-dioxaborolane (278 mg, 1.1 mmol) in dioxane (4 mL) was added bis(triphenylphosphine)palladium(II) chloride (77 mg, 109.5 μmol) and potassium acetate (215 mg, 2.2 mmol) at 25° C. under N₂ atmosphere. Then the mixture was heated to 80° C., and stirred for 16 h under nitrogen atmosphere. The mixture was allowed to cool down to 25° C. Then the mixture was diluted with ethyl acetate (100 mL) and washed with brine (4×100 mL), dried over anhydrous sodium sulfate. The organic phase was concentrated under reduced pressure. The residue was purified by silica gel column chromatography, eluted with 30˜35% ethyl acetate in petroleum ether to afford 1-isopropyl-2-[4-[(4,4,5,5-tetramethyl-1,3,2-dioxaborolan-2-yl)methyl]phenyl]-4-(trifluoromethyl)imidazole (250 mg, 634.14 μmol, 58% yield) as a light-yellow solid. MS: m/z=395.15 [M+H]⁺. ¹H NMR (400 MHz, Chloroform-d) δ 7.48-7.39 (m, 3H), 7.31 (d, J=8.0 Hz, 2H), 4.65-4.59 (m, 1H), 2.37 (s, 2H), 1.47 (d, J=6.8 Hz, 6H), 1.26 (s, 12H).

### 2-[[5-(4-cyclopropyl-6-methoxy-pyrimidin-5-yl)-3-[[4-[I-isopropyl-4-(trifluoromethyl)imidazol-2-yl]phenyl]methyl]pyrazolo[4,3-d]pyrimidin-1-yl]methoxy]ethyl-trimethyl-silane

To a solution of 2-[[3-bromo-5-(4-cyclopropyl-6-methoxy-pyrimidin-5-yl)pyrazolo[4,3-d]pyrimidin-1-yl]methoxy]ethyl-trimethyl-silane (160 mg, 335.13 μmol) and 1-isopropyl-2-[4-[(4,4,5,5-tetramethyl-1,3,2-dioxaborolan-2-yl)methyl]phenyl]-4-(trifluoromethyl)imidazole (198 mg, 502 μmol) in toluene (2 mL) and water (0.4 mL) were added anhydrous potassium phosphate (142 mg, 670 μmol) and bis(triphenylphosphine)palladium(II) chloride (23.5 mg, 33.5 μmol) at 25° C. under nitrogen atmosphere. Then the mixture was heated to 90° C., and stirred for 16 h under nitrogen atmosphere. The mixture was allowed to cool down to 25° C. Then the mixture was diluted with ethyl acetate (200 mL) and washed with brine (4×200 mL), dried over anhydrous sodium sulfate. The organic phase was concentrated under reduced pressure. The residue was purified by chromatography on silica gel elute with 60-70% ethyl acetate in petroleum ether to afford 2-[[5-(4-cyclopropyl-6-methoxy-pyrimidin-5-yl)-3-[[4-[1-isopropyl-4-(trifluoromethyl)imidazol-2-yl]phenyl]methyl]pyrazolo[4,3-d]pyrimidin-1-yl]methoxy]ethyl-trimethyl-silane (84.1 mg, 126.51 μmol, 37% yield) as a yellow solid. MS: m/z=665.55 [M+H]⁺.

### 5-(4-cyclopropyl-6-methoxy-pyrimidin-5-yl)-3-[[4-[1-isopropyl-4-(trifluoromethyl)imidazol-2-yl]phenyl]methyl]-1H-pyrazolo[4,3-d]pyrimidine

To a solution of 2-[[5-(4-cyclopropyl-6-methoxy-pyrimidin-5-yl)-3-[[4-[1-isopropyl-4-(trifluoromethyl)imidazol-2-yl]phenyl]methyl]pyrazolo[4,3-d]pyrimidin-1-yl]methoxy]ethyl-trimethyl-silane (80 mg, 120.34 μmol) in dichloromethane (4 mL) was added trifluoroacetic acid (5.92 g, 51.92 mmol, 4.00 mL) at 0° C. under nitrogen atmosphere. Then the reaction mixture was stirred at 25° C. for 2 h. The reaction solution was concentrated under reduced pressure. The mixture was added ammonium hydroxide (4 mL, 28% ammonia in water) and tetrahydrofuran (4 mL). The mixture was stirred at 25° C. for 30 minutes. The reaction solution was concentrated under vacuum. The residue was purified by silica gel column chromatography, eluted with 80˜90% ethyl acetate in petroleum ether to give a crude product. Then, the crude product was purified by RP-Flash with the following conditions: Column: C18 spherical Column, 20-35 μm, 100A, 80 g; Mobile Phase A: Water (5 mM aq. NH₄HCO₃), Mobile Phase B: MeCN; Flow rate: 50 mL/min; Gradient: 5% B to 5% B in 5 min, 5% B to 40% B in 10 min, 40% B to 40% B in 4 min, 40% B to 95% B in 10 min; Detector: UV 254 & 210 nm; RT: 17 min. The collected fractions were combined, concentrated and then lyophilized overnight to give 5-(4-cyclopropyl-6-methoxy-pyrimidin-5-yl)-3-[[4-[1-isopropyl-4-(trifluoromethyl)imidazol-2-yl]phenyl]methyl]-1H-pyrazolo[4,3-d]pyrimidine (12 mg, 22.45 μmol, 19% yield) as a white solid. MS: m/z=535.25 [M+H]⁺. ¹H NMR (400 MHz, Chloroform-d) δ 13.79 (s, 1H), 9.17 (s, 1H), 8.67 (s, 1H), 7.65-7.55 (m, 4H), 7.52 (s, 1H), 4.69-4.63 (m, 1H), 4.54 (s, 2H), 3.90 (s, 3H), 1.67-1.60 (m, 1H), 1.52 (d, J=6.8 Hz, 6H), 1.25-1.16 (m, 2H), 0.86-0.77 (m, 2H). ¹⁹F NMR (376 MHz, Chloroform-d) δ −62.05.

### Example 7. Synthesis of [2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol (Compound 49), 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (Compound 65) and 2-(4-cyclopropyl-6-methoxy-pyrimidin-5-yl)-5-methyl-7-[[4-[1-methyl-4-(trifluoromethyl)imidazol-2-yl]phenyl]methyl]pyrrolo[3,2-d]pyrimidine (Compound 39)

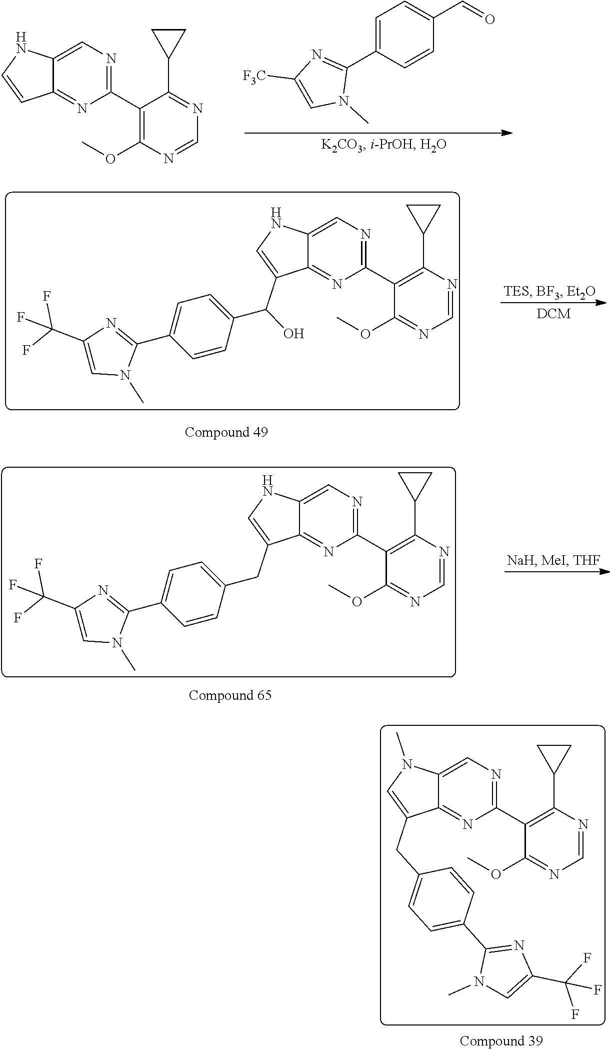 [SMILES: COc1ncnc(C2CC2)c1-c1ncc2[nH]ccc2n1 ; Cn1cc(C(F)(F)F)nc1-c1ccc(C=O)cc1 ; COc1ncnc(C2CC2)c1-c1ncc2[nH]cc(C(O)c3ccc(-c4nc(C(F)(F)F)cn4C)cc3)c2n1 ; COc1ncnc(C2CC2)c1-c1ncc2[nH]cc(Cc3ccc(-c4nc(C(F)(F)F)cn4C)cc3)c2n1 ; COc1ncnc(C2CC2)c1-c1ncc2c(n1)c(Cc1ccc(-c3nc(C(F)(F)F)cn3C)cc1)cn2C]

### [2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol

To a solution of 2-(4-cyclopropyl-6-methoxy-pyrimidin-5-yl)-5H-pyrrolo[3,2-d]pyrimidine (163 mg, 609.82 umol) in isopropyl alcohol (2 mL) was added 4-[1-methyl-4-(trifluoromethyl)imidazol-2-yl]benzaldehyde (187 mg, 732 umol) at 0° C. under nitrogen atmosphere and then stirred at 0° C. for 10 min. To the above mixture were added potassium carbonate (101 mg, 732 umol) and water (2 mL). The resulting mixture was stirred at 50° C. for 16 hr. The reaction solution was detected by TLC and LCMS. The resulted mixture was diluted water (20 mL), extracted with ethyl acetate (3×30 mL). The combined organic layers were washed with brine (30 mL), dried over anhydrous sodium sulfate. After filtration, the filtrate was concentrated under reduced pressure. The residue was purified by Prep-TLC (CH₂Cl₂: MeOH=10:1) to give 110 mg crude. The crude product was purified by Prep-HPLC with the following conditions (Column: XBridge Prep OBD C18 Column, 30×150 mm 5 um; Mobile Phase A: Water (10 mmol/L NH₄HCO₃), Mobile Phase B: MeCN; Flow rate: 60 mL/min; Gradient: 20% B to 30% B in 8 min; Detector: UV 254/220 nm; RT1: 7.33 min) to give [2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol (50 mg, 95.87 μmol, 13% yield) as a white solid. MS: m/z=522.35 [M+H]⁺. ¹H NMR (400 MHz, Chloroform-d) δ 9.92 (s, 1H), 8.99 (s, 1H), 8.68 (s, 1H), 7.70-7.60 (m, 4H), 7.38 (d, J=0.8 Hz, 1H), 7.04 (d, J=2.4 Hz, 1H), 6.41 (s, 1H), 4.38 (s, 1H), 3.94 (s, 3H), 3.80 (s, 3H), 1.73-1.62 (m, 1H), 1.23 (s, 2H), 0.92-0.84 (m, 2H). ¹⁹F NMR (376 MHz, Chloroform-d) δ −62.51.

### 2-(4-cyclopropyl-6-methoxy-pyrimidin-5-yl)-7-[[4-[-methyl-4-(trifluoromethyl)imidazol-2-yl]phenyl]methyl]-5H-pyrrolo[3,2-d]pyrimidine

To a stirred mixture of [2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol (50 mg, 95.8 μmol) in dichloromethane (0.5 mL) were added boron trifluoride etherate (272 mg, 1.9 mmol, 241 μL) and triethyl silicane (223 mg, 1.9 mmol) at 0° C. under nitrogen atmosphere. The resulting mixture was stirred at 0° C. for 16 hr. The reaction mixture was quenched by saturated aq. NaHCO₃ (10 mL) and then extracted with dichloromethane (3×10 mL). The combined organic layers were washed with brine (3×10 mL), dried over anhydrous sodium sulfate. After filtration, the filtrate was concentrated under reduced pressure. The residue was purified by Prep-TLC, eluted with 9% methanol in dichloromethane to give 30 mg crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 20 mL/min; Gradient: 5% B to 5% B in 5 min, 5% B to 28% B in 10 min, 28% B to 28% B in 3 min, 28% B to 95% B in 5 min; Detector: UV 254 & 210 nm; RT: 17 min. The collected fractions were combined, concentrated and then lyophylized to give 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (16 mg, 31.65 μmol, 33% yield) as a white solid. MS: m/z=506.35 [M+H]⁺. ¹H NMR (400 MHz, Chloroform-d) δ 10.70 (s, 1H), 8.90 (d, J=4.8 Hz, 1H), 8.65 (d, J=4.0 Hz, 1H), 7.55-7.49 (m, 2H), 7.44-7.36 (m, 3H), 7.10 (d, J=2.0 Hz, 1H), 4.27 (s, 2H), 3.91 (s, 3H), 3.79 (s, 3H), 1.69-1.66 (m, 1H), 1.24-1.16 (m, 2H), 0.87-0.78 (m, 2H).

### 2-(4-cyclopropyl-6-methoxy-pyrimidin-5-yl)-5-methyl-7-[[4-[1-methyl-4-(trifluoromethyl)imidazol-2-yl]phenyl]methyl]pyrrolo[3,2-d]pyrimidine

To a stirred mixture of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (50 mg, 98.91 μmol) in THF (1 mL) was added sodium hydride (5.9 mg, 148 μmol, 60% purity) under nitrogen atmosphere at 0° C. The resulting mixture was stirred for 1 hr at 0° C. To the above mixture was added methyl iodide (21 mg, 148 μmol, 9.2 μL) under nitrogen atmosphere at 25° C. The resulting mixture was stirred at 25° C. for 2 hr. The reaction was quenched by the addition of ammonium chloride (30 mL). The resulting mixture was extracted with ethyl acetate (3×10 mL). The combined organic layers were washed with brine (3×10 mL), dried over anhydrous sodium sulfate. After filtration, the filtrate was concentrated under reduced pressure. The residue was purified by Prep-TLC, eluted with 10% methanol in dichloromethane to give 35 mg crude product. The crude product was purified by RP-Flash with the following conditions: Column: C18 spherical Column, 20-35 μm, 100A, 20 g; Mobile Phase A: Water (5 mM aq. NH₄HCO₃), Mobile Phase B: MeCN; Flow rate: 60 mL/min; Gradient: 5% B to 5% B in 5 min, 5% B to 55% B in 10 min, 55% B to 55% B in 3 min, 55% B to 95% B in 5 min; Detector: UV 254 & 210 nm; RT: 17 min. The collected fractions were combined, concentrated and then lyophilized to give 2-(4-cyclopropyl-6-methoxy-pyrimidin-5-yl)-5-methyl-7-[[4-[1-methyl-4-(trifluoromethyl)imidazol-2-yl]phenyl]methyl]pyrrolo[3,2-d]pyrimidine (22.6 mg, 43.50 μmol, 44% yield) as an off-white solid. MS: m/z=520.25 [M+H]⁺. ¹H NMR (400 MHz, Chloroform-d) δ 8.98 (s, 1H), 8.69 (d, J=3.6 Hz, 1H), 7.58 (d, J=8.0 Hz, 2H), 7.47 (d, J=8.0 Hz, 2H), 7.33 (s, 1H), 7.10 (s, 1H), 4.28 (s, 2H), 3.95 (s, 3H), 3.91 (s, 3H), 3.78 (s, 3H), 1.77-1.66 (m, 1H), 1.30-1.20 (m, 2H), 0.92-0.83 (m, 2H); ¹⁹F NMR (376 MHz, Chloroform-d) δ −62.72.

### Example 8. The Synthesis of 2-(2-isopropylphenyl)-7-[[4-[1-methyl-4-(trifluoromethyl)imidazol-2-yl]phenyl]methyl]-5H-pyrrolo[3,2-d]pyrimidine (Compound 10)

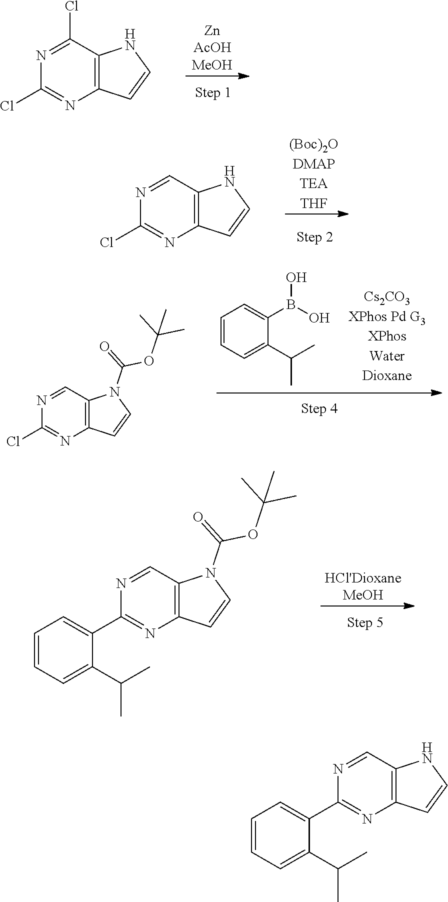

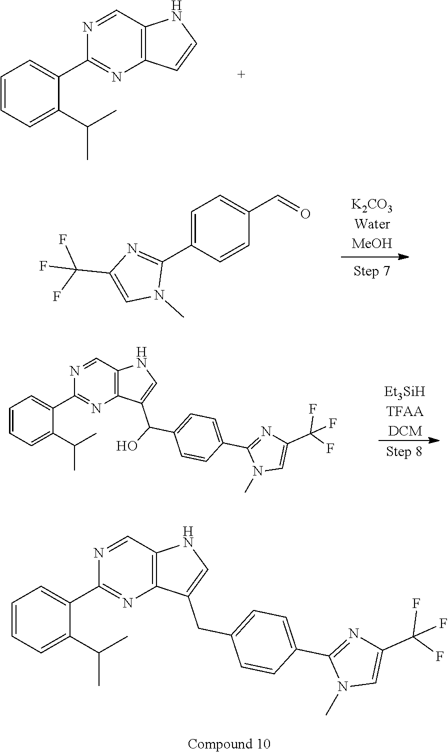 [SMILES: CC(C)c1ccccc1-c1ncc2[nH]ccc2n1 ; Cn1cc(C(F)(F)F)nc1-c1ccc(C=O)cc1 ; CC(C)c1ccccc1-c1ncc2[nH]cc(C(O)c3ccc(-c4nc(C(F)(F)F)cn4C)cc3)c2n1 ; CC(C)c1ccccc1-c1ncc2[nH]cc(Cc3ccc(-c4nc(C(F)(F)F)cn4C)cc3)c2n1]

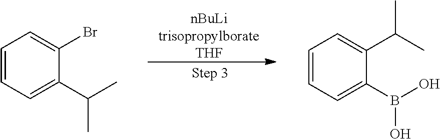

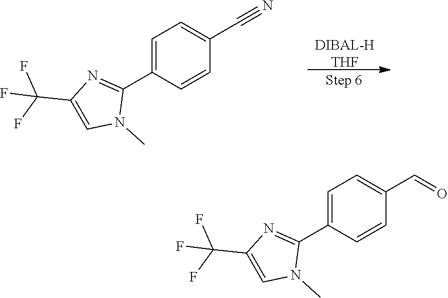 [SMILES: Cn1cc(C(F)(F)F)nc1-c1ccc(C#N)cc1 ; Cn1cc(C(F)(F)F)nc1-c1ccc(C=O)cc1]

### Step 1: The Synthesis of 2-chloro-5H-pyrrolo[3,2-d]pyrimidine

To a solution of 2,4-dichloro-5H-pyrrolo[3,2-d]pyrimidine (1.00 g, 5.32 mmol) in MeOH (20.0 mL) and acetic acid (2.01 mL) Zinc dust (1.39 g, 21.3 mmol) was added. The resulting mixture was stirred at 65° C. for 3 hr. The reaction mixture was filtered and the filtrate was concentrated in vacuo. The residue was diluted with water (5.00 mL) and the precipitate formed was filtered off to afford 2-chloro-5H-pyrrolo[3,2-d]pyrimidine (350 mg, 2.28 mmol, 42.85% yield) as a light-yellow solid which was used in the next step without further purification. 1H NMR (400 MHz, DMSO-d₆) δ 6.56 (d, 1H), 7.98 (d, 1H), 8.79 (s, 1H), 12.04 (br, 1H). LCMS(ESI): [M+H]+ m/z: calcd 154.01; found 154.0.

### Step 2: The Synthesis of tert-butyl 2-chloropyrrolo[3,2-d]pyrimidine-5-carboxylate

To a solution of 2-chloro-5H-pyrrolo[3,2-]pyrimidine (200 mg, 1.30 mmol) in THE (5.00 mL) DMAP (15.9 mg, 130 μmol) and TEA (145 mg, 1.43 mmol, 200 μL) were added. The reaction mixture was stirred for 5 min at room temperature, then di-tert-butyl dicarbonate (341 mg, 1.56 mmol, 359 μL) was added dropwise. The resulting mixture was stirred at room temperature for 14 hr. The reaction mixture was poured into H₂O (50.0 mL) and the obtained mixture was extracted with EtOAc (2×30.0 mL). The combined organic layers were washed with Brine (2×20.0 mL), dried over anhydrous sodium sulfate and concentrated in vacuo to afford tert-butyl 2-chloropyrrolo[3,2-d]pyrimidine-5-carboxylate (220 mg, 867 μmol, 67% yield) as a yellow solid which was used in the next steps without further purification.

1H NMR (400 MHz, CDCl₃) δ 1.68 (s, 9H), 6.70 (d, 1H), 7.99 (d, 1H), 9.21 (s, 1H).

### Step 3: The synthesis of (2-isopropylphenyl)boronic Acid

To a solution of 1-bromo-2-isopropyl-benzene (11.0 g, 55.3 mmol) in THF (100 mL) n-Butyllithium, 2.2M in hexane (25.1 mL, 55.3 mmol) was added dropwise at −80° C. The reaction mixture was stirred for 1 hr at −80° C., then triisopropylborate (11.4 g, 60.8 mmol, 14.0 mL) in MTBE (10.0 mL) was added. The resulting mixture was stirred for 6 hr, at ambient temperature. The reaction mixture was diluted with water (50 mL) and volatiles were removed in vacuo. The residue was acidified with diluted sulfuric acid, the precipitate formed was filtered off, dried on air and recrystallized from hexane to give (2-isopropylphenyl)boronic acid (2.00 g, 12.2 mmol, 22% yield) as a white solid.

1H NMR (400 MHz, DMSO-d) δ 1.19 (d, 6H), 3.12-3.28 (m, 1H), 7.06-7.14 (m, 1H), 7.22-7.32 (m, 3H), 8.06 (br, 1H).

### Step 4: The synthesis of tert-butyl 2-(2-isopropylphenyl)pyrrolo[3,2-d]pyrimidine-5-carboxylate

To a degassed mixture of Water (20.0 mL) and Dioxane (80.0 mL) tert-butyl 2-chloropyrrolo[3,2-d]pyrimidine-5-carboxylate (4.00 g, 15.8 mmol), (2-isopropylphenyl)boronic acid (3.36 g, 20.5 mmol), Cesium carbonate (10.3 g, 31.5 mmol) and XPhos Pd G3 (1.00 g, 1.18 mmol) were added in an inert atmosphere at room temperature. The resulting mixture was stirred at 100° C. for 10 hr. The reaction mixture was cooled to room temperature and concentrated in vacuo. The residue was diluted with H₂O (10.0 mL) and extracted with EtOAc (3×15.0 mL). The combined organic layers were dried over anhydrous sodium sulfate, filtered and treated with palladium scavenger SiliaMetS® Dimercaptotriazine (2.0 g) for 6 hr, at room temperature. The resulting mixture was filtered and concentrated in vacuo to afford tert-butyl 2-(2-isopropylphenyl)pyrrolo[3,2-d]pyrimidine-5-carboxylate (4.50 g, crude) as a light-yellow solid which was used in the next step without further purification.

1H NMR (500 MHz, CDCl₃) δ 1.14-1.24 (m, 6H), 1.70-1.81 (m, 9H), 3.38-3.49 (m, 1H), 6.81 (s, 1H), 7.20-7.30 (m, 2H), 7.35-7.49 (m, 2H), 7.50-7.59 (m, 1H), 9.47 (br, 1H) LCMS(ESI): [M+H]+m/z: calcd 338.21; found 338.2.

### Step 5: The Synthesis of 2-(2-isopropylphenyl)-5H-pyrrolo[3,2-d]pyrimidine

To a solution of tert-butyl 2-(2-isopropylphenyl)pyrrolo[3,2-d]pyrimidine-5-carboxylate (4.50 g, 13.3 mmol) in MeOH (45.0 mL) hydrogen chloride solution, 4.0M in dioxane, (30 mL) was added dropwise. The reaction mixture was stirred for 30 hr, at 25° C., then an additional portion of hydrogen chloride solution, 4.0M in dioxane, (20.0 mL) was added and stirring was continued for 19 hr. The reaction mixture was concentrated in vacuo. The residue (3.50 g) was triturated with acetone-MTBE (1:1, 20.0 mL) to afford 2-(2-isopropylphenyl)-5H-pyrrolo[3,2-d]pyrimidine (3.00 g, 10.96 mmol, HCl salt, 69.5% yield from tert-butyl 2-chloropyrrolo[3,2-d]pyrimidine-5-carboxylate) as a light-yellow solid which was used in the next step without further purification.

1H NMR (500 MHz, DMSO) δ 1.16-1.17 (d, 6H), 3.16-3.18 (m, 1H), 6.92 (m, 1H), 7.34-7.38 (m, 1H), 7.51-7.55 (m, 3H), 8.43-8.44 (m, 1H), 9.28 (s, 1H), 13.03 (br, 1H).

LCMS(ESI): [M+H]+m/z: calcd 238.15; found 238.0.

### Step 6: The Synthesis of 4-[1-methyl-4-(trifluoromethyl)imidazol-2-yl]benzaldehyde

To a solution of 4-[1-methyl-4-(trifluoromethyl)imidazol-2-yl]benzonitrile (3.00 g, 11.9 mmol) in THF (150 mL) Diisobutylaluminum hydride (5.94 g, 41.80 mmol, 7.45 mL) was added dropwise at 0° C. The resulting mixture was stirred for 5 hr, at 25° C. The reaction mixture was poured into 1M HCl (200 mL) and extracted with EtOAc (150×2 mL). The combined organic layer was washed with Brine (50.0 mL), dried over anhydrous sodium sulfate and concentrated in vacuo to afford 4-[1-methyl-4-(trifluoromethyl)imidazol-2-yl]benzaldehyde (2.80 g, 11.0 mmol, 98% yield) as yellow oil which was used in the next step without further purification. 1H NMR (400 MHz, CDCl₃) δ 3.82 (s, 3H), 7.83 (d, 2H), 7.98 (d, 2H), 10.07 (s, 1H). LCMS(ESI): [M+H]+m/z: calcd 255.08; found 255.2.

### Step 7: The Synthesis of [2-(2-isopropylphenyl)-5H-pyrrolo[3,2-d]pyrimidin-7-yl]-[4-[1-methyl-4-(trifluoromethyl)imidazol-2-yl]phenyl]methanol

To a mixture of 2-(2-isopropylphenyl)-5H-pyrrolo[3,2-d]pyrimidine (300 mg, 1.10 mmol, HCl salt) and 4-[1-methyl-4-(trifluoromethyl)imidazol-2-yl]benzaldehyde (491 mg, 1.64 mmol) in Water (2.00 mL) and MeOH (2.00 mL) Potassium carbonate (437 mg, 3.16 mmol) was added. The reaction mixture was stirred at 25° C. for 48 hr. then an additional portion of K₂CO₃ (175 mg, 1.26 mmol) was added. The resulting mixture was stirred for 24 hr, and K₂CO₃ (175 mg, 1.26 mmol) was added again. The resulting mixture was stirred for an additional 24 hr. The reaction mixture was concentrated in vacuo. The residue was diluted with DCM (15.0 mL) and washed with Brine (5.00 mL), dried over anhydrous sodium sulfate and concentrated in vacuo to afford [2-(2-isopropylphenyl)-5H-pyrrolo[3,2-d]pyrimidin-7-yl]-[4-[1-methyl-4-(trifluoromethyl)imidazol-2-yl]phenyl]methanol (380 mg, crude) as a light-yellow solid which was used in the next step without further purification.

LCMS(ESI): [M+H]+ m/z: calcd 492.23; found 492.2.

Step 8: The synthesis of 2-(2-isopropylphenyl)-7-[[4-[1-methyl-4-(trifluoromethyl)imidazol-2-yl]phenyl]methyl]-5H-pyrrolo[3,2-d]pyrimidine

To a solution of [2-(2-isopropylphenyl)-5H-pyrrolo[3,2-d]pyrimidin-7-yl]-[4-[1-methyl-4-(trifluoromethyl)imidazol-2-yl]phenyl]methanol (380 mg, 541 μmol) in DCM (5.00 mL) Triethylsilane (315 mg, 2.71 mmol, 432 μL) and Trifluoroacetic acid (247 mg, 2.16 mmol, 167 μL) were added. The reaction mixture was stirred at ambient temperature for 20 hr. An aliquot showed about 30% conversion. Triethylsilane (160 mg, 1.35 mmol, 216 μL) and Trifluoroacetic acid (124 mg, 1.08 mmol, 84 μL) were added again and stirring was continued for 30 hr, at ambient temperature. The reaction mixture was poured into saturated NaHCO₃ (10.0 mL) and DCM (10.0 mL). The organic layer was separated, dried over Na₂SO₄ and concentrated in vacuo. The residue was subjected to HPLC (2-10 min 45-60% water-ACN, +0.1% vol, of 25% aq. NH₃, 30 mL/min, column: YMC-Actus Triart C18, 100×20 mm, 5 μm) to afford 2-(2-isopropylphenyl)-7-[[4-[1-methyl-4-(trifluoromethyl)imidazol-2-yl]phenyl]methyl]-5H-pyrrolo[3,2-d]pyrimidine (90.0 mg, 189 μmol, 35% yield) as a light-yellow solid. 1H NMR (500 MHz, CDCl₃) δ (ppm) 1.15 (d, 6H), 3.48-3.32 (m, 1H), 3.71 (s, 3H), 4.19 (s, 2H), 7.01 (s, 1H), 7.23 (t, 1H), 7.30-7.44 (m, 5H) 7.48 (d, 2H), 7.56 (d, 1H), 8.64 (s, 1H), 10.90 (s, 1H).

LCMS(ESI): [M+H]+ m/z: calcd 476.24; found 476.2.

### Example 9. Synthesis of 2-[2-(2-isopropylphenyl)-7-[[4-[1-methyl-4-(trifluoromethyl)imidazol-2-yl]phenyl]methyl]pyrrolo[3,2-d]pyrimidin-5-yl]ethanol (Compound 57)

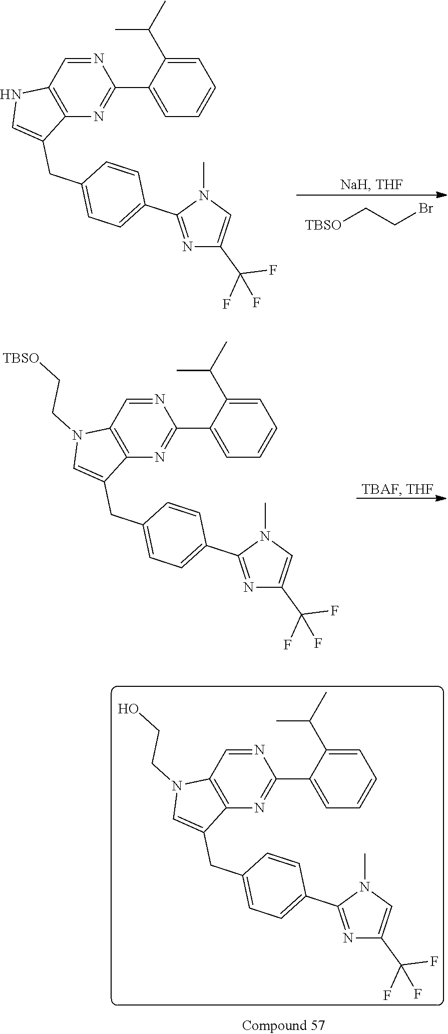

### tert-butyl-[2-[2-(2-isopropylphenyl)-7-[[4-[1-methyl-4-(trifluoromethyl)imidazol-2-yl]phenyl]methyl]pyrrolo[3,2-d]pyrimidin-5-yl]ethoxy]-dimethyl-silane

To a solution of 2-(2-isopropylphenyl)-7-[[4-[1-methyl-4-(trifluoromethyl)imidazol-2-yl]phenyl]methyl]-5H-pyrrolo[3,2-d]pyrimidine (32 mg, 67.30 μmol) in THF (1 mL) was added sodium hydride (5.4 mg, 135 μmol, 60% purity) at 0° C., then stirred at 25° C. for 1 hr under nitrogen atmosphere. The above mixture was added 2-bromoethoxy-tert-butyl-dimethyl-silane (19 mg, 81 μmol, 17.3 μL) at 0° C. The resulting mixture was stirred for 1 hr at 25° C. under nitrogen. The residue was purified by Prep-TLC, eluted with 30% ethyl acetate in petroleum ether to give tert-butyl-[2-[2-(2-isopropylphenyl)-7-[[4-[-methyl-4-(trifluoromethyl)imidazol-2-yl]phenyl]methyl]pyrrolo[3,2-d]pyrimidin-5-yl]ethoxyl]-dimethyl-silane (15 mg, 23.67 umol, 35% yield) as an off-white solid. MS: m/z=634.25 [M+H]⁺.

### 2-[2-(2-isopropylphenyl)-7-[[4-[1-methyl-4-(trifluoromethyl)imidazol-2-yl]phenyl]methyl]pyrrolo[3,2-d]pyrimidin-5-yl]ethanol

A solution of tert-butyl-[2-[2-(2-isopropylphenyl)-7-[[4-[1-methyl-4-(trifluoromethyl)imidazol-2-yl]phenyl]methyl]pyrrolo[3,2-d]pyrimidin-5-yl]ethoxy]-dimethyl-silane (15 mg, 23.67 μmol) and TBAF (25 mg, 95 μmol) in THF (0.5 mL) was stirred at 60° C. for 4 hr under nitrogen atmosphere. The resulted reaction mixture was concentrated under reduced pressure. The residue was purified by RP-Flash with the following conditions: Column: C18 spherical Column, 20-35 μm, 100A, 40 g; Mobile Phase A: Water, Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 33 min. The collected fractions were combined and concentrated under reduced pressure to afford 2-[2-(2-isopropylphenyl)-7-[[4-[1-methyl-4-(trifluoromethyl)imidazol-2-yl]phenyl]methyl]pyrrolo[3,2-d]pyrimidin-5-yl]ethanol (5.6 mg, 10.78 μmol, 45% yield) as an off-white solid. MS: m/z=520.25 [M+H]⁺. ¹H NMR (400 MHz, Chloroform-d) δ 9.02 (s, 1H), 7.61 (d, J=7.6 Hz, 1H), 7.55 (d, J=8.0 Hz, 2H), 7.50-7.38 (m, 4H), 7.32-7.26 (m, 3H), 4.30 (t, J=4.8 Hz, 2H), 4.24 (s, 2H), 3.94 (t, J=4.8 Hz, 2H), 3.76 (s, 3H), 3.54-3.43 (m, 1H), 1.24 (d, J=6.8 Hz, 6H). ¹⁹F NMR (376 MHz, Chloroform-d) δ −62.69.

### Example 10. Synthesis of 2-(2-isopropylphenyl)-5-(2-methoxyethyl)-7-[[4-[1-methyl-4-(trifluoromethyl)imidazol-2-yl]phenyl]methyl]pyrrolo[3,2-d]pyrimidine (Compound 83)

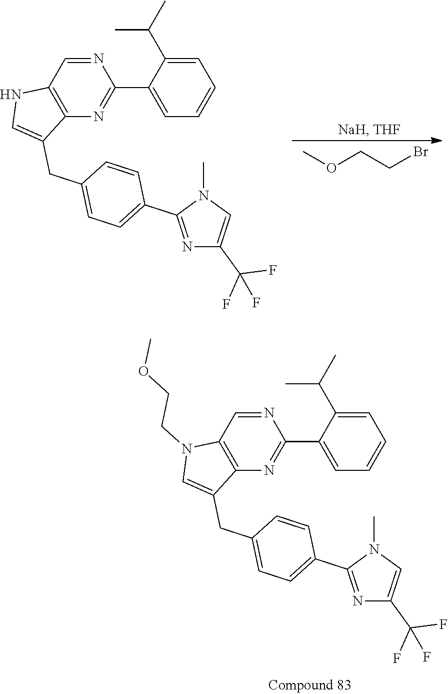

### 2-(2-isopropylphenyl)-5-(2-methoxyethyl)-7-[[4-[1-methyl-4-(trifluoromethyl)imidazol-2-yl]phenyl]methyl]pyrrolo[3,2-d]pyrimidine

To a solution of 2-(2-isopropylphenyl)-7-[[4-[1-methyl-4-(trifluoromethyl)imidazol-2-yl]phenyl]methyl]-5H-pyrrolo[3,2-d]pyrimidine (30 mg, 63.09 μmol) in THF (1 mL) was added NaH (5 mg, 126 μmol, 60% purity) at 0° C. then stirred at 25° C. for 1 hr under nitrogen and then 1-bromo-2-methoxy-ethane (13 mg, 95 μmol, 8.9 uL) was added at 0° C. The resulting mixture was stirred at 25° C. for 1 hr under nitrogen, then was quenched by saturated ammonium chloride (20 mL) and extracted with ethyl acetate (3×15 mL). The combined organic layers were washed with brine (15 mL), dried over anhydrous sodium sulfate. After filtration, the filtrate was concentrated under reduced pressure. The residue was purified by RP-Flash with the following conditions: Column: C18 spherical Column, 20-35 um, 100A, 40 g; Mobile Phase A: Water, Mobile Phase B: MeCN; Flow rate: 50 mL/min; Gradient: 0% B to 0% B in 5 min, 0% B to 65% B in 20 min, 50% B to 95% B in 10 min; Detector: UV 254 & 210 nm; RT: 31 min. The collected fractions were combined and concentrated under reduced pressure to afford 2-(2-isopropylphenyl)-5-(2-methoxyethyl)-7-[[4-[1-methyl-4-(trifluoromethyl)imidazol-2-yl]phenyl]methyl]pyrrolo[3,2-d]pyrimidine (6.8 mg, 12.74 μmol, 20% yield) as an off-white solid. MS: m/z=534.20 [M+H]⁺. ¹H NMR (400 MHz, Chloroform-d) δ 9.02 (d, J=9.6 Hz, 1H), 7.70-7.63 (m, 1H), 7.61-7.57 (m, 2H), 7.51-7.39 (m, 4H), 7.34-7.25 (m, 3H), 4.35 (s, 2H), 4.27 (s, 2H), 3.80-3.71 (m, 5H), 3.57-3.48 (m, 1H), 3.37 (s, 3H), 1.26 (d, J=7.2 Hz, 61H). ¹⁹F NMR (376 MHz, Chloroform-d) δ −62.71.

### Example 11. Synthesis of 2-(2-isopropylphenyl)-N-[4-[1-methyl-4-(trifluoromethyl)imidazol-2-yl]phenyl]-5H-pyrrolo[3,2-d]pyrimidin-7-amine (Compound 62)

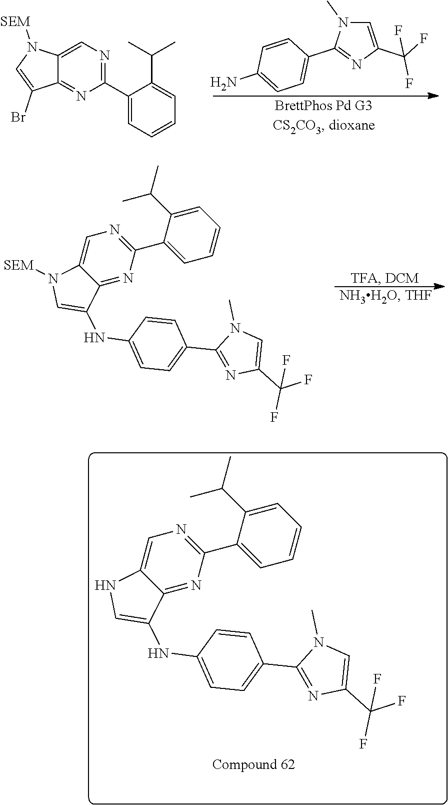

### 2-(2-isopropylphenyl)-N-[4-[1-methyl-4-(trifluoromethyl)imidazol-2-yl]phenyl]-5-(2-trimethylsilylethoxymethyl)pyrrolo[3,2-d]pyrimidin-7-amine

To a solution of 2-[[7-bromo-2-(2-isopropylphenyl)pyrrolo[3,2-d]pyrimidin-5-yl]methoxy]ethyl-trimethyl-silane (80 mg, 179.19 μmol) and 4-[1-methyl-4-(trifluoromethyl)imidazol-2-yl]aniline (65 mg, 269 μmol) in Dioxane (2 mL) were added cesium carbonate (117 mg, 358 μmol) and methanesulfonato(2-dicyclohexylphosphino-3,6-Dimethoxy-2′,4′,6-Tri-i-Propyl-1,1′-Biphenyl-2-yl)Palladium (16 mg, 18 μmol) at 25° C. The resulting mixture was stirred at 100° C. for 16 hr under N₂ atmosphere. The residue was purified by Prep-TLC (PE:EtOAc=1:1). The collected fractions were concentrated under reduced pressure to afford 2-(2-isopropylphenyl)-N-[4-[1-methyl-4-(trifluoromethyl)imidazol-2-yl]phenyl]-5-(2-trimethylsilylethoxymethyl)pyrrolo[3,2-d]pyrimidin-7-amine (90 mg, 148.33 μmol, 83% yield) as a brown oil. MS: m/z=607.30 [M+H]+.

### 2-(2-isopropylphenyl)-N-[4-[1-methyl-4-(trifluoromethyl)imidazol-2-yl]phenyl]-5H-pyrrolo[3,2-d]pyrimidin-7-amine

To a solution of 2-(2-isopropylphenyl)-N-[4-[1-methyl-4-(trifluoromethyl)imidazol-2-yl]phenyl]-5-(2-trimethylsilylethoxymethyl)pyrrolo[3,2-d]pyrimidin-7-amine (80 mg, 131 μmol) in methylene chloride (1 mL) was added trifluoroacetic acid (4 mL) at 0° C., and then stirred at 25° C. for 2 hrs. The reaction solution was concentrated under reduced pressure to give an intermediate. To the intermediate was added ammonium hydroxide (28% in H₂O, 3 mL) and tetrahydrofuran (3 mL). The resulting mixture was stirred at 25° C. for 1 hr under N₂ atmosphere and then concentrated under reduced pressure. The residue was purified by Prep-HPLC with the following conditions: Column: XBridge Shield RP18 OBD Column, 30×150 mm, 5 μm; Mobile Phase A: 10 mM aq. NH₄HCO₃, Mobile Phase B: MeCN; Flow rate: 60 mL/min; Gradient: 38% B to 65% B in 7 min; Detector: UV 254 nm; RT: 7.2 min. The collected fractions were combined, concentrated under reduced pressure and then lyophilized overnight to give 2-(2-isopropylphenyl)-N-[4-[1-methyl-4-(trifluoromethyl)imidazol-2-yl]phenyl]-5H-pyrrolo[3,2-d]pyrimidin-7-amine (20.4 mg, 42.81 μmol, 32% yield) as a red solid. MS: m/z=477.20 [M+H]⁺. ¹H NMR (400 MHz, Chloroform-d) δ 11.13 (br., 1H), 9.02 (s, 1H), 7.60-7.54 (m, 1H), 7.48-7.35 (m, 4H), 7.30-7.22 (m, 3H), 6.99 (d, J=8.4 Hz, 2H), 6.25 (br., 1H), 3.77 (s, 3H), 3.46-3.35 (m, 1H), 1.19 (d, J=6.8 Hz, 6H); ¹⁹F NMR (376 MHz, Chloroform-d) δ −61.20.

### Example 12. Synthesis of [2-(2-isopropylphenyl)-5H-pyrrolo[3,2-d]pyrimidin-7-yl]-[4-[1-methyl-4-(trifluoromethyl)imidazol-2-yl]phenyl]methanone (Compound 86)

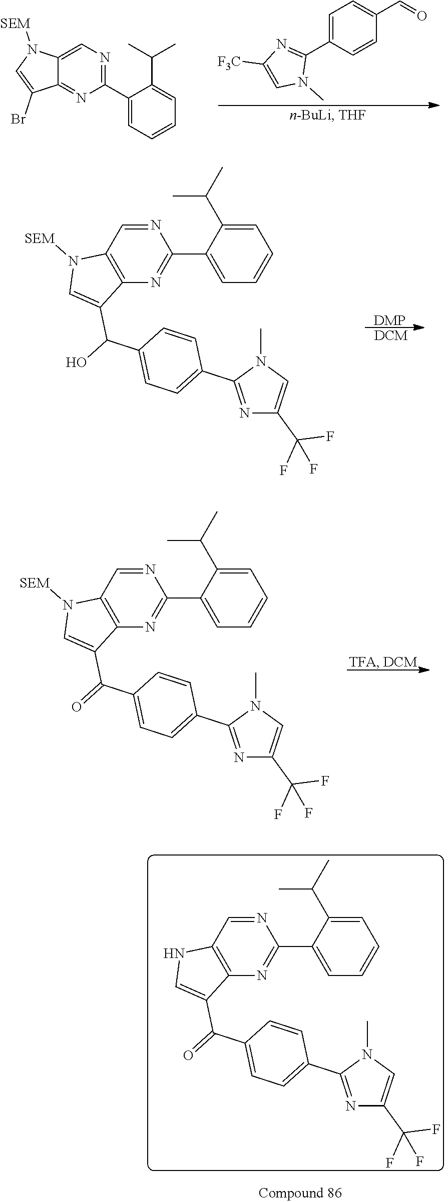 [SMILES: CC(C)c1ccccc1-c1ncc2c(n1)c(Br)cn2COCC[Si](C)(C)C ; Cn1cc(C(F)(F)F)nc1-c1ccc(C=O)cc1 ; CC(C)c1ccccc1-c1ncc2c(n1)c(C(O)c1ccc(-c3nc(C(F)(F)F)cn3C)cc1)cn2COCC[Si](C)(C)C ; CC(C)c1ccccc1-c1ncc2c(n1)c(C(=O)c1ccc(-c3nc(C(F)(F)F)cn3C)cc1)cn2COCC[Si](C)(C)C ; CC(C)c1ccccc1-c1ncc2[nH]cc(C(=O)c3ccc(-c4nc(C(F)(F)F)cn4C)cc3)c2n1]

### [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol

To a solution of 2-[[7-bromo-2-(2-isopropylphenyl)pyrrolo[3,2-d]pyrimidin-5-yl]methoxy]ethyl-trimethyl-silane (500 mg, 1.12 mmol) and 4-[1-methyl-4-(trifluoromethyl)imidazol-2-yl]benzaldehyde (285 mg, 1.1 mmol) in THF (5 mL), n-Butyllithium (2.5 M in hexane, 0.536 mL, 1.34 mmol) was added dropwise at −78° C. then stirred at 78° C. for 2 hr under N₂ atmosphere. The resulted reaction mixture was quenched by the addition saturated aqueous ammonium chloride (50 mL) at 0° C., and then extracted with EA (3×20 mL). The combined organic fractions were dried over anhydrous sodium sulfate. After filtration, the filtrate was concentrated under reduced pressure. The residue was purified by silica gel column chromatography, eluted with 1%-60% EA in Petroleum ether. The collected fractions were combined and concentrated under reduced pressure to give [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol (150 mg, 241.25 μmol, 21% yield) as a yellow solid. MS: m/z=622.25 [M+H]⁺. ¹H NMR (400 MHz, Chloroform-d) δ 9.11 (s, 1H), 7.68-7.66 (m, 4H), 7.53-7.41 (m, 2H), 7.37-7.31 (m, 2H), 7.29 (s, 1H), 7.07 (s, 1H), 6.39 (s, 1H), 5.48 (s, 2H), 3.80 (s, 3H), 3.57-3.49 (m, 3H), 1.32-1.25 (m, 6H), 0.94 (t, J=8.4 Hz, 2H), −0.02 (s, 9H).

### [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanone

A solution of [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol (150 mg, 241.25 μmol) and Dess-Martin Reagent (205 mg, 483 μmol) in DCM (10 mL) was stirred at 25° C. for 1 hr under N₂ atmosphere. The resulted reaction mixture was quenched by saturated sodium bicarbonate solution (40 mL) at 0° C., and then extracted with EA (3×20 mL). The combined organic fractions were dried over anhydrous sodium sulfate. After filtration, the filtrate was concentrated under reduced pressure. The residue was purified by Prep-TLC (PE EtOAc=1:1). The collected fractions were concentrated under reduced pressure to afford [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanone (80 mg, 129.08 μmol, 53% yield) as an off-white solid. MS: m/z=620.25 [M+H]+. ¹H NMR (400 MHz, Chloroform-d) δ 9.24 (s, 1H), 8.25 (s, 1H), 8.14 (d, J=8.0 Hz, 2H), 8.09-8.03 (m, 1H), 8.01-7.90 (m, 1H), 7.77 (d, J=8.4 Hz, 2H), 7.70 (s, 1H), 7.54-7.38 (m, 1H), 7.28-7.16 (m, 2H), 5.69 (s, 2H), 3.80 (s, 3H), 3.69-3.61 (m, 2H), 3.57-3.46 (m, 1H), 1.13 (d, J=6.8 Hz, 6H), 1.05-0.92 (m, 2H), 0.09 (s, 9H).

### [2-(2-isopropylphenyl)-5H-pyrrolo[3,2-d]pyrimidin-7-yl]-[4-[1-methyl-4-(trifluoromethyl)imidazol-2-yl]phenyl]methanone

To a solution of [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanone (80 mg, 129.08 umol) in DCM (1 mL) was added trifluoroacetic acid (4 mL) at 0° C., and then stirred at 25° C. for 2 hr. The resulted reaction was concentrated under reduced pressure. The residue was purified by Prep-HPLC with the following conditions: Column: XBridge Prep OBD C18 Column, 30×150 mm 5 μm; Mobile Phase A: Water (10 mM aq. NH₄HCO₃+0.1% NH₃·H₂O), Mobile Phase B: MeCN; Flow rate: 60 mL/min; Gradient: 35% B to 65% B in 8 min; Detector: UV 254 & 210 nm; RT: 7.23 min; The collected fractions were combined, concentrated under reduced pressure and then lyophilized overnight to give [2-(2-isopropylphenyl)-5H-pyrrolo[3,2-d]pyrimidin-7-yl]-[4-[1-methyl-4-(trifluoromethyl)imidazol-2-yl]phenyl]methanone (18.3 mg, 37.39 μmol, 29% yield) as an off-white solid. MS: m/z=490.15 [M+H]⁺. ¹H NMR (400 MHz, DMSO-d₆) δ 12.79 (br., 1H), 9.17 (s, 1H), 8.57 (s, 1H), 8.05-7.98 (m, 3H), 7.92-7.86 (m, 2H), 7.59-7.52 (m, 1H), 7.45-7.34 (m, 2H), 7.28-7.19 (m, 1H), 3.85 (s, 3H), 3.53-3.42 (m, 1H), 1.07 (d, J=6.8 Hz, 6H); ¹⁹F NMR (376 MHz, DMSO-d₆) δ −60.63.

### Example 13. Synthesis of [2-(2-isopropylphenyl)-5H-pyrrolo[3,2-d]pyrimidin-7-yl]-[4-[1-methyl-4-(trifluoromethyl)imidazol-2-yl]phenyl]methanol (Compound 1)

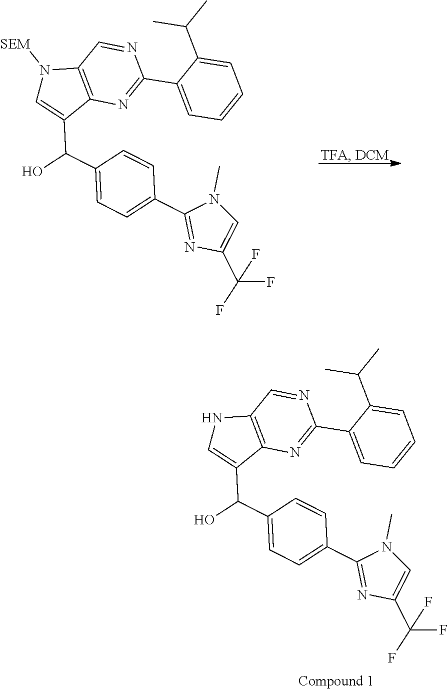 [SMILES: CC(C)c1ccccc1-c1ncc2c(n1)c(C(O)c1ccc(-c3nc(C(F)(F)F)cn3C)cc1)cn2COCC[Si](C)(C)C ; CC(C)c1ccccc1-c1ncc2[nH]cc(C(O)c3ccc(-c4nc(C(F)(F)F)cn4C)cc3)c2n1]

To a solution of [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol (20 mg, 32 umol) in dichloromethane (1 mL) was added trifluoroacetic acid (4 mL) at 0° C., and then stirred at 25° C. for 2 hr. The resulted mixture was concentrated under reduced pressure. The residue was purified by RP-Flash with the following condition: Column: C18 spherical Column, 20-35 μm, 100A, 120 g; Mobile Phase A: Water, Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 33 min. The collected fractions were combined, concentrated under reduced pressure and then lyophilized overnight to give [2-(2-isopropylphenyl)-5H-pyrrolo[3,2-d]pyrimidin-7-yl]-[4-[1-methyl-4-(trifluoromethyl)imidazol-2-yl]phenyl]methanol (5.2 mg, 10.58 μmol, 33% yield) as an off-white solid. MS: m/z=492.20 [M+H]⁺. ¹H NMR (400 MHz, Chloroform-d) δ 10.06 (br., 1H), 8.83 (s, 1H), 7.68-7.58 (m, 5H), 7.51-7.40 (m, 2H), 7.38 (s, 1H), 7.34-7.28 (m, 1H), 7.05 (s, 1H), 6.36 (s, 1H), 3.78 (s, 3H), 3.54-3.43 (m, 1H), 1.24 (d, J=6.8 Hz, 6H). ¹⁹F NMR (376 MHz, Chloroform-d) δ −62.48.

### Example 14. Synthesis of 2-(2-isopropylphenyl)-7-[methoxy-[4-[1-methyl-4-(trifluoromethyl)imidazol-2-yl]phenyl]methyl]-5H-pyrrolo[3,2-d]pyrimidine (Compound 73)

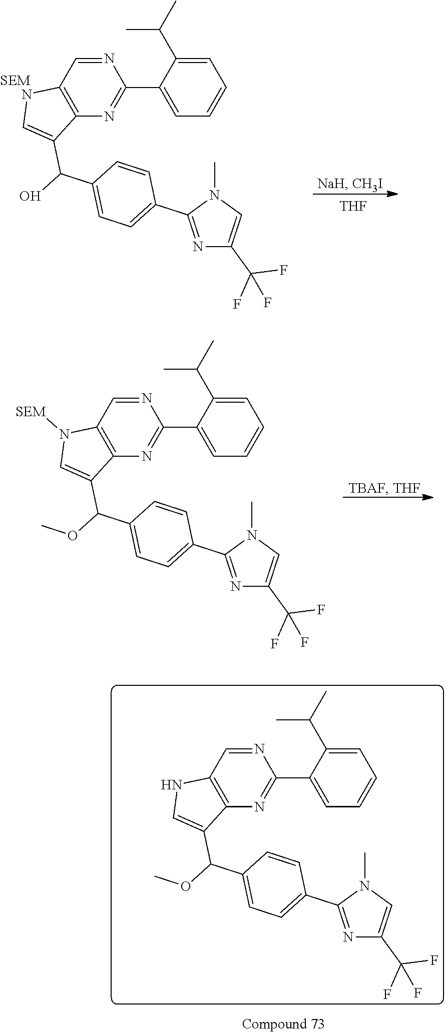

### 2-[[2-(2-isopropylphenyl)-7-[methoxy-[4-[1-methyl-4-(trifluoromethyl)imidazol-2-yl]phenyl]methyl]pyrrolo[3,2-d]pyrimidin-5-yl]methoxy]ethyl-trimethyl-silane

To a solution of [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol (50 mg, 80.42 μmol) in THF (0.5 mL) was added sodium hydride (2 mg, 80 μmol, 60%) partially at 0° C. The resulting mixture was further stirred at 0° C. for 1 h, then iodomethane (134 mg, 97 μmol, 6.0 μL) was added. After stirred at 25° C. for 8 h, the reaction was quenched by ice-water (10 mL) and extracted with ethyl acetate (10 mL×2). The organic fractions were dried over anhydrous sodium sulfate. After filtration, the filtrate was concentrated under reduced pressure. The residue was purified by Prep-TLC (PE:EtOAc=1:1). The collected fractions were concentrated under reduced pressure afford 2-[[2-(2-isopropylphenyl)-7-[methoxy-[4-[1-methyl-4-(trifluoromethyl)imidazol-2-yl]phenyl]methyl]pyrrolo[3,2-d]pyrimidin-5-yl]methoxy]ethyl-trimethyl-silane (46.8 mg, 73.61 μmol, 90% yield) as an off-white solid. MS: m/z=636.20 [M+H]⁺. ¹H NMR (400 MHz, Chloroform-d) δ 9.07 (s, 1H), 7.72-7.62 (m, 5H), 7.53-7.40 (m, 2H), 7.36-7.28 (m, 3H), 5.97 (s, 1H), 5.51 (s, 2H), 3.79 (s, 3H), 3.64-3.54 (m, 3H), 3.51 (s, 3H), 1.29 (d, J=6.8 Hz, 6H), 0.98-0.86 (m, 2H), 0.09 (s, 9H).

### 2-(2-isopropylphenyl)-7-[methoxy-[4-[-methyl-4-(trifluoromethyl)imidazol-2-yl]phenyl]methyl]-5H-pyrrolo[3,2-d]pyrimidine

A solution of [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol (30 mg, 48.25 μmol) and tetrabutylammonium fluoride (13 mg, 48 μmol) in THF (0.5 mL) was stirred at 60° C. for 4 hr under nitrogen atmosphere. The resulted mixture was concentrated under reduced pressure. The residue was purified by RP-Flash with the following conditions: Column: C18 spherical Column, 20-35 μm, 100A, 40 g; Mobile Phase A: 10 mM aq. NH₄HCO₃, Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 33 min. The collected fractions were combined, concentrated under reduced pressure and then lyophilized overnight to afford 2-(2-isopropylphenyl)-7-[methoxy-[4-[1-methyl-4-(trifluoromethyl)imidazol-2-yl]phenyl]methyl]-5H-pyrrolo[3,2-d]pyrimidine (3.6 mg, 7.12 μmol, 15% yield) as an off-white solid. MS: m/z=506.20. (H NMR (400 MHz, Chloroform-d) δ 9.96 (br., 1H), 8.76 (s, 1H), 7.68-7.58 (m, 5H), 7.51-7.39 (m, 2H), 7.36 (s, 1H), 7.33-7.30 (m, 1H), 5.98 (s, 1H), 3.78 (s, 3H), 3.60-3.51 (m, 1H), 3.49 (s, 3H), 1.30-1.20 (m, 6H); ¹⁹F NMR (376 MHz, Chloroform-d) δ −62.59.

### Example 15. Synthesis of 5-(2-isopropylphenyl)-3-[4-[1-methyl-4-(trifluoromethyl)imidazol-2-yl]phenoxy]-1H-pyrrolo[2,3-c]pyridine (Compound 14)

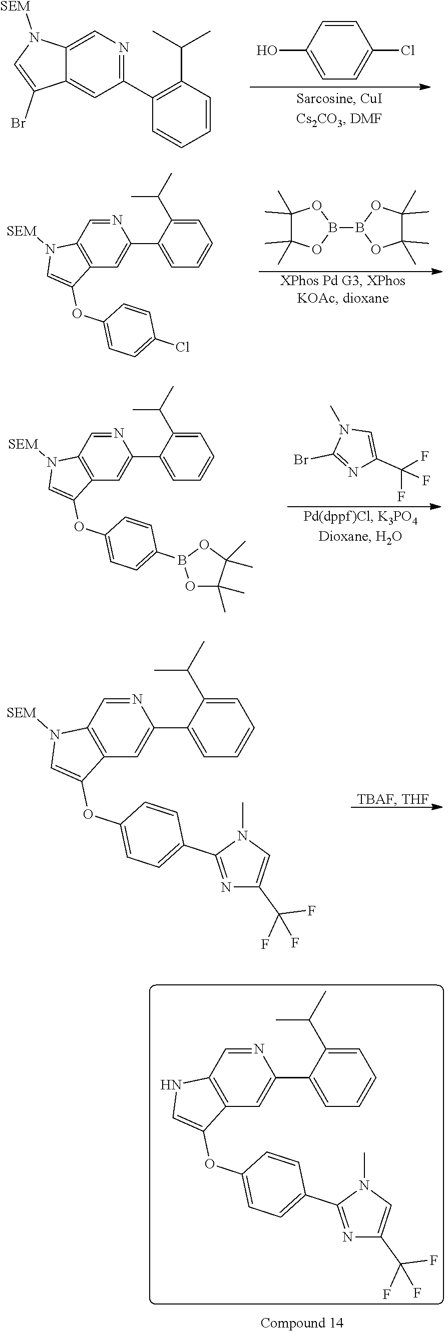

### 2-[[3-(4-chlorophenoxy)-5-(2-isopropylphenyl)pyrrolo[2,3-c]pyridin-1-yl]methoxy]ethyl-trimethyl-silane

To a solution of 2-[[3-bromo-5-(2-isopropylphenyl)pyrrolo[2,3-c]pyridin-1-yl]methoxy]ethyl-trimethyl-silane (500 mg, 1.12 mmol) and 4-chlorophenol (289 mg, 2.2 mmol, 220 μL) in N,N-dimethylformamide (5 mL) was added cesium carbonate (556 mg, 1.7 mmol), sarcosine (40 mg, 445 umol) and cuprous iodide (11.4 mg, 60.3 μmol). The resulting mixture was stirred at 110° C. for 16 h under nitrogen, then filtered and concentrated under reduced pressure. The residue was purified by silica gel column chromatography, eluted with 1%-50% ethyl acetate in petroleum ether to give 2-[[3-(4-chlorophenoxy)-5-(2-isopropylphenyl)pyrrolo[2,3-c]pyridin-1-yl]methoxy]ethyl-trimethyl-silane (40 mg, 81.12 μmol, 7% yield) as a brown oil. MS: m/z=493.15, 495.15 [M+H]⁺.

### 2-[[5-(2-isopropylphenyl)-3-[4-(4,4,5,5-tetramethyl-1,3,2-dioxaborolan-2-yl)phenoxy]pyrrolo[2,3-c]pyridin-1-yl]methoxy]ethyl-trimethyl-silane

To a solution of 2-[[3-(4-chlorophenoxy)-5-(2-isopropylphenyl)pyrrolo[2,3-c]pyridin-1-yl]methoxy]ethyl-trimethyl-silane (40 mg, 81.12 μmol) and 4,4,5,5-tetramethyl-2-(4,4,5,5-tetramethyl-1,3,2-dioxaborolan-2-yl)-1,3,2-dioxaborolane (31 mg, 121.7 μmol) in dioxane (1 mL) was added potassium acetate (16 mg, 162 μmol), dicyclohexyl(2′,4′,6′-triisopropyl-[1,1′-biphenyl]-2-yl)phosphane (3.9 mg, 8.1 μmol) and methanesulfonato(2-dicyclohexylphosphino-2′,4′,6′-tri-i-propyl-1,1′-biphenyl)(2-amino-1,1′-biphenyl-2-yl)palladium(I) (6.9 mg, 8.1 μmol) at 25° C. The resulting mixture was stirred at 100° C. for 16 h under nitrogen and then filtered and concentrated under reduced pressure. The residue was purified by silica gel column chromatography, eluted with 1˜50% ethyl acetate in petroleum ether to give 2-[[5-(2-isopropylphenyl)-3-[4-(4,4,5,5-tetramethyl-1,3,2-dioxaborolan-2-yl)phenoxy]pyrrolo[2,3-c]pyridin-1-yl]methoxy]ethyl-trimethyl-silane (40 mg, 68.42 μmol, 84% yield) as a brown oil. MS: m/z=585.20 [M+H]⁺.

### 2-[[5-(2-isopropylphenyl)-3-[4-[1-methyl-4-(trifluoromethyl)imidazol-2-yl]phenoxy]pyrrolo[2,3-c]pyridin-1-yl]methoxy]ethyl-trimethyl-silane

To a solution of 2-[[5-(2-isopropylphenyl)-3-[4-(4,4,5,5-tetramethyl-1,3,2-dioxaborolan-2-yl)phenoxy]pyrrolo[2,3-c]pyridin-1-yl]methoxy]ethyl-trimethyl-silane (40 mg, 68.42 μmol) and 2-bromo-1-methyl-4-(trifluoromethyl)imidazole (23.5 mg, 102.6 umol) in dioxane (1 mL) and water (0.2 mL) was added potassium phosphate (44 mg, 205 μmol) and 1,1′-bis(diphenylphosphino)ferrocene-palladium(II)dichloride dichloromethane complex (5.6 mg, 6.8 μmol) at 25° C. The resulting mixture was stirred at 100° C. for 16 h under nitrogen. The resulting mixture was filtered and then concentrated under reduced pressure. The residue was purified by silica gel column chromatography, eluted with 1˜50% ethyl acetate in petroleum ether to give 2-[[5-(2-isopropylphenyl)-3-[4-[1-methyl-4-(trifluoromethyl)imidazol-2-yl]phenoxy]pyrrolo[2,3-c]pyridin-1-yl]methoxy]ethyl-trimethyl-silane (30 mg, 49.44 μmol, 72% yield) as a brown oil. MS: m/z=607.20 [M+H]⁺.

### 5-(2-isopropylphenyl)-3-[4-[1-methyl-4-(trifluoromethyl)imidazol-2-yl]phenoxy]-1H-pyrrolo[2,3-c]pyridine

A solution of 2-[[5-(2-isopropylphenyl)-3-[4-[1-methyl-4-(trifluoromethyl)imidazol-2-yl]phenoxy]pyrrolo[2,3-c]pyridin-1-yl]methoxy]ethyl-trimethyl-silane (30 mg, 49.44 umol) and TBAF (12.9 mg, 49.4 umol, 14.3 μL) in THE (1 mL) was stirred at 60° C. for 4 hr under nitrogen. The resulting mixture was concentrated under reduced pressure. The residue was purified by RP-Flash with the following conditions: Column: C18 spherical Column, 20-35 um, 100A, 40 g; Mobile Phase A: Water. Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 20 min. The collected fractions were combined, concentrated under reduced pressure and then lyophilized overnight to afford 5-(2-isopropylphenyl)-3-[4-[1-methyl-4-(trifluoromethyl)imidazol-2-yl]phenoxy]-1H-pyrrolo[2,3-c]pyridine (8.9 mg, 18.68 μmol, 37% yield) as an off-white solid. MS: m/z=477.15 [M+H]⁺. ¹H NMR (400 MHz, Methanol-d₄) δ 8.78 (s, 1H), 7.70-7.60 (m, 4H), 7.44-7.32 (m, 2H), 7.24-7.17 (m, 5H), 3.76 (s, 3H), 3.01-2.94 (m, 1H), 1.10 (d, J=6.8 Hz, 6H). ¹⁹F NMR (376 MHz, Methanol-d₄) δ −62.64.

### Example 16. Synthesis of 2-(2-isopropylphenyl)-7-[4-[1-methyl-4-(trifluoromethyl)imidazol-2-yl]phenoxy]-5H-pyrrolo[3,2-d]pyrimidine (Compound 2)

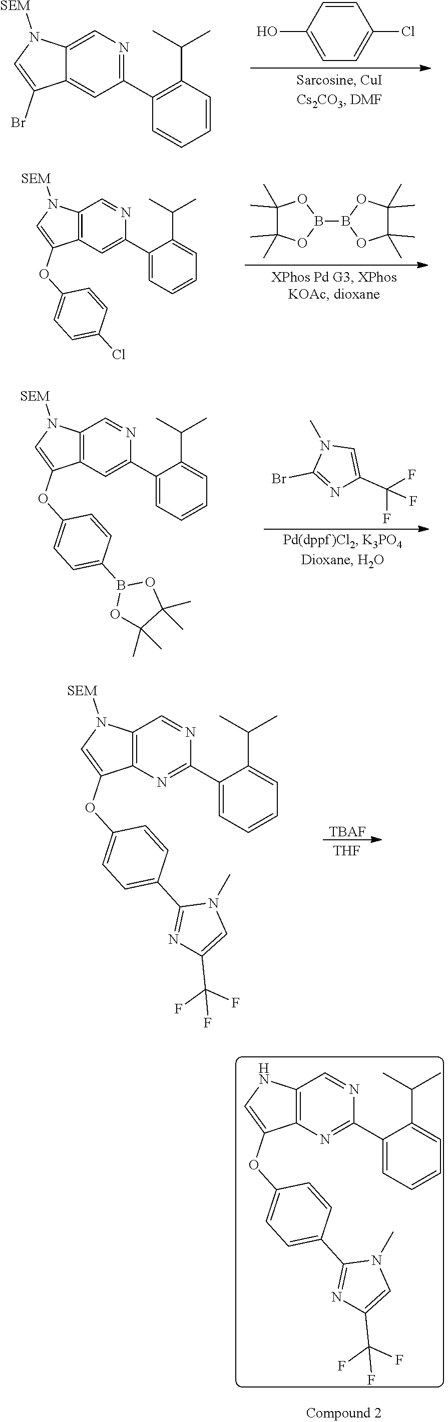

### 2-[[7-(4-chlorophenoxy)-2-(2-isopropylphenyl)pyrrolo[3,2-d]pyrimidin-5-yl]methoxy]ethyl-trimethyl-silane

To a solution of 2-[[7-bromo-2-(2-isopropylphenyl)pyrrolo[3,2-d]pyrimidin-5-yl]methoxy]ethyl-trimethyl-silane (prepared as in Example 11) (500 mg, 1.12 mmol) and 4-chlorophenol (216 mg, 1.7 mmol, 165 μL) in N,N-dimethylformamide (5 mL) was added cesium carbonate (547 mg, 1.68 mmol), sarcosine (20 mg, 224 μmol) and cuprous iodide (85 mg, 448 μmol) at 25° C. The resulting mixture was stirred at 110° C. for 16 h under nitrogen atmosphere.

The resulting mixture was filtered and then concentrated under reduced pressure. The residue was purified by silica gel column chromatography, eluted with 1˜50% ethyl acetate in petroleum ether to give 2-[[7-(4-chlorophenoxy)-2-(2-isopropylphenyl)pyrrolo[3,2-d]pyrimidin-5-yl]methoxy]ethyl-trimethyl-silane (50 mg, 101.19 μmol, 9% yield) as a brown oil. MS: m/z=494.15, 496.15 [M+H]⁺. ¹H NMR (400 MHz, Chloroform-d) δ 9.15 (s, 1H), 7.69-7.61 (m, 1H), 7.52-7.30 (m, 3H), 7.28-7.10 (m, 3H), 7.05-6.97 (m, 2H), 5.57 (s, 2H), 3.63-3.49 (m, 2H), 3.47-3.36 (m, 1H), 1.30-1.09 (m, 6H), 1.03-0.91 (m, 2H), 0.12-0.05 (m, 9H).

### 2-[[2-(2-isopropylphenyl)-7-[[4-(4,4,5,5-tetramethyl-1,3,2-dioxaborolan-2-yl)phenoxy]pyrrolo[3,2-d]pyrimidin-5-yl]methoxy]ethyl-trimethyl-silane

To a solution of 7-(4-chlorophenoxy)-2-(2-isopropylphenyl)-5H-pyrrolo[3,2-d]pyrimidine (45 mg, 123.68 μmol) and 4,4,5,5-tetramethyl-2-(4,4,5,5-tetramethyl-1,3,2-dioxaborolan-2-yl)-1,3,2-dioxaborolane (47 mg, 185.5 μmol) in dioxane (1 mL) were added potassium acetate (12.14 mg, 123.68 μmol), methanesulfonato(2-dicyclohexylphosphino-2′,4′,6′-tri-i-propyl-1,1′-biphenyl)(2′-amino-1,1′-biphenyl-2-yl)palladium(II) (105 mg, 124 μmol) and dicyclohexyl(2′,4′,6′-triisopropyl-[1,1′-biphenyl]-2-yl)phosphane (59 mg, 124 μmol) at 25° C. The resulting mixture was stirred at 100° C. for 16 h under nitrogen then filtered and concentrated under reduced pressure. The residue was purified by silica gel column chromatography, eluted with 1˜50% ethyl acetate in petroleum ether to give 2-[[2-(2-isopropylphenyl)-7-[4-(4,4,5,5-tetramethyl-1,3,2-dioxaborolan-2-yl)phenoxy]pyrrolo[3,2-d]pyrimidin-5-yl]methoxy]ethyl-trimethyl-silane (40 mg, 68.30 umol, 55% yield) as a brown oil. MS: ml/z=585.90 [M+H]⁺.

### 2-[[2-(2-isopropylphenyl)-7-[4-[-methyl-4-(trifluoromethyl)imidazol-2-yl]phenoxy]pyrrolo[3,2-d]pyrimidin-5-yl]methoxy]ethyl-trimethyl-silane

To a solution of 2-[[2-(2-isopropylphenyl)-7-[4-(4,4,5,5-tetramethyl-1,3,2-dioxaborolan-2-yl)phenoxy]pyrrolo[3,2-d]pyrimidin-5-yl]methoxy]ethyl-trimethyl-silane (40 mg, 68.30 μmol) and 2-bromo-1-methyl-4-(trifluoromethyl)imidazole (23.5 mg, 102.5 umol) in dioxane (0.6 mL) and water (0.1 mL) were added potassium phosphate (44 mg, 205 μmol) and 1,1′-bis(diphenylphosphino)ferrocene-palladium(II)dichloride dichloromethane complex (5.6 mg, 6.8 μmol) at 25° C. The resulting mixture was stirred at 100° C. for 16 h under N₂, then filtered and concentrated under reduced pressure. The residue was purified by silica gel column chromatography, eluted with 1%-50% ethyl acetate in petroleum ether to give 2-[[2-(2-isopropylphenyl)-7-[4-[1-methyl-4-(trifluoromethyl)imidazol-2-yl]phenoxy]pyrrolo[3,2-d]pyrimidin-5-yl]methoxy]ethyl-trimethyl-silane (30 mg, 49.36 μmol, 72% yield) as a brown oil. MS: m/z=608.15 [M+H]⁺.

### 2-(2-isopropylphenyl)-7-[4-[1-methyl-4-(trifluoromethyl)imidazol-2-yl]phenoxy]-5H-pyrrolo[3,2-d]pyrimidine

A solution of 2-[[2-(2-isopropylphenyl)-7-[4-[1-methyl-4-(trifluoromethyl)imidazol-2-yl]phenoxy]pyrrolo[3,2-d]pyrimidin-5-yl]methoxy]ethyl-trimethyl-silane (30 mg, 49.36 μmol) and TBAF (52 mg, 197 μmol, 57 μL) in THF (1 mL) was stirred at 60° C. for 4 hr under nitrogen. The resulting mixture was concentrated under reduced pressure. The residue was purified by RP-Flash with the following conditions: Column: C18 spherical Column, 20-35 um, 100A, 40 g; Mobile Phase A: Water, Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 25 min. The collected fractions were combined, concentrated under reduced pressure and then lyophilized overnight to afford 2-(2-isopropylphenyl)-7-[4-[1-methyl-4-(trifluoromethyl)imidazol-2-yl]phenoxy]-5H-pyrrolo[3,2-d]pyrimidine (11.4 mg, 23.88 μmol, 48% yield) as an off-white solid. MS: m/z=478.15 [M+H]⁺. ¹H NMR (400 MHz, Chloroform-d) δ 11.41 (br., 1H), 9.07 (s, 1H), 7.56 (d, J=7.2 Hz, 1H), 7.50 (d, J=8.8 Hz, 2H), 7.42-7.33 (m, 3H), 7.28-7.20 (m, 1H), 7.07 (d, J=8.8 Hz, 2H), 6.97 (s, 1H), 3.77 (s, 3H), 3.41-3.30 (m, 1H), 1.10 (d, J=6.8 Hz, 6H). ¹⁹F NMR (376 MHz, Chloroform-d) δ −62.36.

### Example 17. Synthesis of [2-(4-cyclopropyl-6-methoxy-pyrimidin-5-yl)-5H-pyrrolo[3,2-d]pyrimidin-7-yl]-[4-[1-isopropyl-4-(trifluoromethyl)imidazol-2-yl]phenyl]methanol (Compound 59)

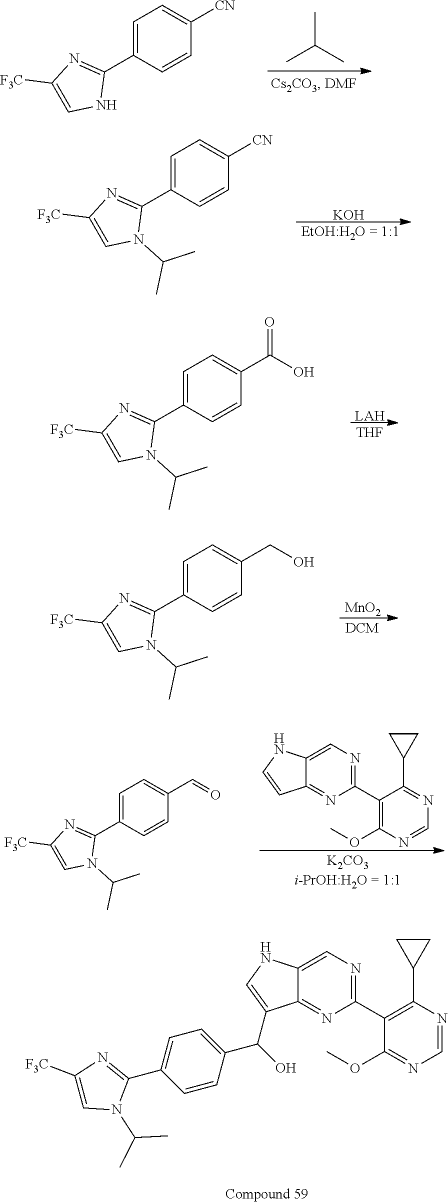

### 4-[1-isopropyl-4-(trifluoromethyl)imidazol-2-yl]benzonitrile

To a solution of 4-[4-(trifluoromethyl)-1H-imidazol-2-yl]benzonitrile (2.96 g 12.48 mmol) in DMF (15 mL) were added 2-iodopropane (2.55 g, 14.98 mmol) and cesium carbonate (4.88 g, 14.98 mmol) at 25° C. The resulting mixture was warmed up to 110° C., and stirred for 18 h then naturally cooled down to 25° C. The resulted mixture was diluted by EA (100 mL×3). The organic layer was washed with brine (100 mL×3), dried over anhydrous sodium sulfate.

After filtration, the filtrate was concentrated under reduced pressure. The residue was purified by silica gel column chromatography, eluted with 0%-25% EtOAc in PE to give 4-[1-isopropyl-4-(trifluoromethyl)imidazol-2-yl]benzonitrile (1.06 g, 3.80 mmol, 30% yield) as a yellow solid. MS: m/z=280.15 [M+H]⁺. ¹H NMR (400 MHz, Chloroform-d) δ 7.85-7.77 (m, 2H), 7.75-7.68 (m, 2H), 7.50 (s, 1H), 4.60-4.54 (m, 1H), 1.51 (d, J=6.8 Hz, 6H).

### 4-[1-isopropyl-4-(trifluoromethyl)imidazol-2-yl]benzoic Acid

To a solution of 4-[1-isopropyl-4-(trifluoromethyl)imidazol-2-yl]benzonitrile (1.06 g, 3.80 mmol) in H₂O (10 mL) and EtOH (10 mL) was added potassium hydroxide (1.06 g, 18.98 mmol) at 25° C. After the resulting solution was stirred for 3 hours at 90° C., it was naturally cooled down to 25° C. The reaction mixture was adjusted pH to 5 with 1 M aq. HCl and then purified by RP-Flash chromatography with the following conditions: Column: C18 spherical, 20-35 um, 100A, 330 g; Mobile Phase A: Water, Mobile Phase B: MeCN; Flow rate: 80 mL/min; Gradient: 5% B to 5% B in 5 min, 5% B to 45% B in 20 min; 45% B to 45% B in 5 min; 45% B to 95% B in 5 min; Detector: UV 254 & 220 nm; RT: 30 min to give 4-[1-isopropyl-4-(trifluoromethyl)imidazol-2-yl]benzoic acid (0.81 g, 2.72 mmol, 71% yield) as an off-white solid. MS: m/z=299.15 [M+H]⁺. ¹H NMR (400 MHz, Chloroform-d) δ 8.24 (d, J=8.0 Hz, 2H), 7.70 (d, J=8.0 Hz, 2H), 7.49 (s, 1H), 4.65-4.58 (m, 1H), 1.51 (d, J=6.8 Hz, 6H).

### (4-(1-isopropyl-4-(trifluoromethyl)-1H-imidazol-2-yl)phenyl)methanol

To a solution of 4-[1-isopropyl-4-(trifluoromethyl)imidazol-2-yl]benzoic acid (680 mg, 2.28 mmol) in THF (8 mL) was added lithium aluminum hydride (113 mg, 2.9 mmol) in several portions at 0° C. The resulting mixture stirred for 1 hour under N₂ atmosphere, then was warmed to room temperature and stirred for 4 hours under N₂ atmosphere. The resulted mixture was quenched by saturated aqueous ammonium chloride (30 mL), was extracted by EA (50 mL×2).

The organic layer was washed with brine (30 mL×2), dried over anhydrous sodium sulfate.

After filtration, the filtrate was concentrated under reduced pressure, which was used directly in the next step without further purification. MS: m/z=285.00 [M+H]⁺.

### 4-[-isopropyl-4-(trifluoromethyl)imidazol-2-yl]benzaldehyde

To a solution of [4-[1-isopropyl-4-(trifluoromethyl)imidazol-2-yl]phenyl]methanol (588 mg, 2.07 mmol) in DCM (15 mL) was added manganese(IV) oxide (1.80 g, 20.68 mmol) at 25° C. The resulting mixture was stirred for 16 hours at 40° C. then naturally cooled down to 25° C. The resulted mixture was filtered through a Celite pad and the filtrate was concentrated under reduced pressure. The residue was purified by silica gel column chromatography, eluted with 0%-10% EtOAc in PE to afford 4-[1-isopropyl-4-(trifluoromethyl)imidazol-2-yl]benzaldehyde (580 mg, 2.05 mmol, 99% yield) as an off-white solid. MS: m/z=283.20 [M+H]⁺. ¹H NMR (400 MHz, Chloroform-d) δ 10.12 (s, 1H), 8.06-7.99 (m, 2H), 7.79-7.77 (m, 2H), 7.50 (s, 1H), 4.65-4.62 (m, J=6.8 Hz, 1H), 1.52 (d, J=6.8 Hz, 6H).

### [2-(4-cyclopropyl-6-methoxy-pyrimidin-5-yl)-5H-pyrrolo[3,2-d]pyrimidin-7-yl]-[4-[1-isopropyl-4-(trifluoromethyl)imidazol-2-yl]phenyl]nethanol

To a solution of 4-[1-isopropyl-4-(trifluoromethyl)imidazol-2-yl]benzaldehyde (579 mg, 2.05 mmol) and 2-(4-cyclopropyl-6-methoxy-pyrimidin-5-yl)-5H-pyrrolo[3,2-d]pyrimidine (548 mg, 2.05 mmol) in i-PrOH (5 mL) and H₂O (5 mL) was added potassium carbonate (340 mg, 2.5 mmol). The resulting solution was stirred for 16 h at 60° C. then naturally cooled down to 25° C. The reaction was diluted by H₂O (50 mL). The mixture solution was extracted by EA (50 mL×3). The organic layer was washed with brine (50 mL×3), then dried over anhydrous sodium sulfate. After filtration, the filtrate was concentrated under reduced pressure. The residue was purified by silica gel column chromatography, eluted with 0%-30% EtOAc in PE to afford a crude product, then the crude product was further purified by RP-Flash chromatography with the following conditions: Column: C18 spherical, 20˜30 um, 100A, 40 g; Mobile Phase A: 10 mM aq. NH₄HCO₃, Mobile Phase B: MeCN; Flow rate: 50 mL/min; Gradient: 5% B to 5% B in 5 min, 5% B to 35% B in 15 min; 35% B to 35% B in 5 min; 35% B to 95% B in 5 min; Detector: UV 254 & 220 nm; RT: 20 min to give [2-(4-cyclopropyl-6-methoxy-pyrimidin-5-yl)-5H-pyrrolo[3,2-d]pyrimidin-7-yl]-[4-[1-isopropyl-4-(trifluoromethyl)imidazol-2-yl]phenyl]methanol (128 mg, 232.92 μmol, 11% yield) as an off-white solid. MS: m/z=550.20 [M+H]⁺; ¹H NMR (400 MHz, Chloroform-d) δ 9.02 (s, 1H), 8.69 (s, 1H), 7.69 (d, J=8.4 Hz, 2H), 7.56 (d, J=8.4 Hz, 2H), 7.47 (s, 1H), 6.43 (s, 1H), 4.61-4.54 (m, 1H), 3.97 (s, 3H), 1.89-1.79 (m, 6H), 1.52 (d, J=6.8 Hz, 2H), 1.35-1.28 (m, 2H), 0.95-0.86 (m, 2H). ¹⁹F NMR (376 MHz, Chloroform-d) δ −62.48.

### Example 18. 2-(4-cyclopropyl-6-methoxy-pyrimidin-5-yl)-5H-pyrrolo[3,2-d]pyrimidin-7-yl]-[4-[1-cyclopropyl-4-(trifluoromethyl)imidazol-2-yl]phenyl]methanol (Compound 19) and 2-(4-cyclopropyl-6-methoxy-pyrimidin-5-yl)-7-[[4-[1-cyclopropyl-4-(trifluoromethyl)imidazol-2-yl]phenyl]methyl]-5H-pyrrolo[3,2-d]pyrimidine (Compound 81)

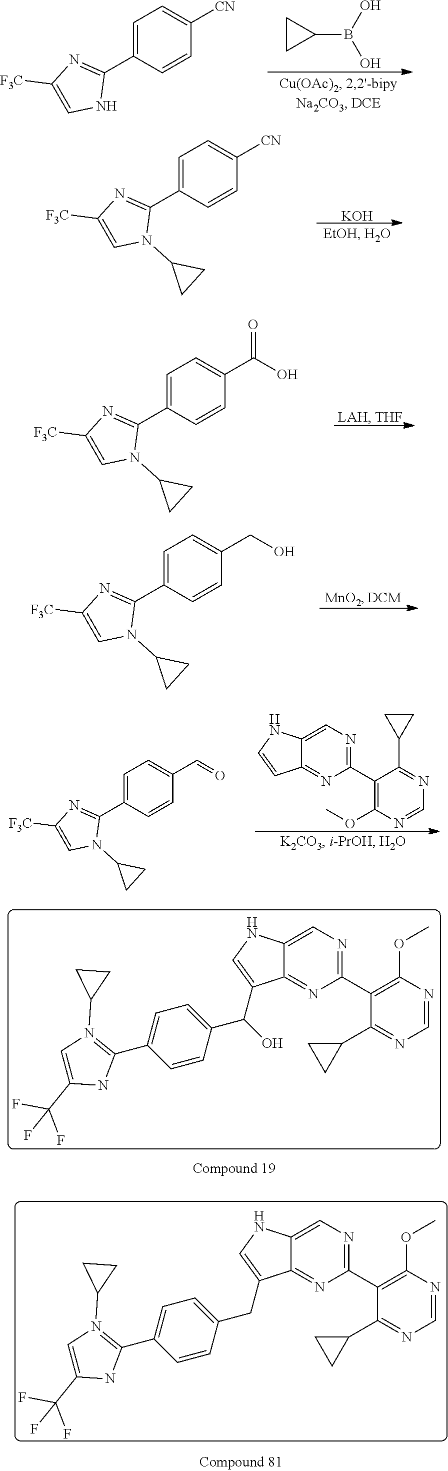

### 4-[1-cyclopropyl-4-(trifluoromethyl)imidazol-2-yl]benzonitrile

To a solution of 4-[4-(trifluoromethyl)-1H-imidazol-2-yl]benzonitrile (370 mg, 1.56 mmol) in 1,2-dichloroethane (10 mL) were added cyclopropylboronic acid (402 mg, 4.7 mmol), cupric acetate (850 mg, 4.6 mmol), 2-(2-pyridyl)pyridine (487 mg, 3.1 mmol) and sodium carbonate (661 mg, 6.2 mmol) at 15 TC. The resulting mixture was stirred at 65° C. for 12 h under air atmosphere then filtrated. The filtrate was concentrated under reduced pressure. The residue was purified by silica gel column chromatography, eluted with 20%-40% ethyl acetate in petroleum ether to afford 4-[1-cyclopropyl-4-(trifluoromethyl)imidazol-2-yl]benzonitrile (80 mg, 288.55 μmol, 18% yield) as a yellow solid. MS: m/z=278.00 [M+H]⁺. ¹H NMR (400 MHz, Chloroform-d) δ 8.07-8.00 (m, 2H), 7.85-7.74 (m, 2H), 7.43 (s, 1H), 3.59-3.53 (m, 1H), 1.19-1.14 (m, 2H), 1.03-0.87 (m, 2H). ¹⁹F NMR (376 MHz, Chloroform-d) δ −62.897

### 4-[1-cyclopropyl-4-(trifluoromethyl)imidazol-2-yl]benzoic Acid

To a solution of 4-[1-cyclopropyl-4-(trifluoromethyl)imidazol-2-yl]benzonitrile (120 mg, 433 μmol) in ethanol (1.5 mL) and water (1.5 mL) was added potassium hydroxide (121 mg, 2.2 mmol) at 15° C., then stirred at 100° C. for 2 h. The reaction solution was adjusted pH to 5-6 using 1 M aq. HCl and then was further purified by RP-Flash chromatography with the following conditions: Column: C18 spherical, 20-35 μm, 100A, 80 g; Mobile Phase A: Water (0.05 mM aq. HCl), Mobile Phase B: MeCN; Flow rate: 60 mL/min; Gradient: 5% B to 5% B in 5 min, 5% B to 15% B in 5 min, 15% B to 40% B in 25 min, 40% B to 95% B in 5 min; Detector: UV 254 & 220 nm; RT: 32 min. The collected fractions were combined, concentrated and then lyophilized overnight to give 4-[1-cyclopropyl-4-(trifluoromethyl)imidazol-2-yl]benzoic acid (105 mg, 354.44 μmol, 81% yield) as a white solid. MS: m/z=296.90 [M+H]⁺. ¹H NMR (400 MHz, Methanol-d₄) δ 8.26-8.18 (m, 2H), 8.03-7.95 (m, 3H), 3.81-3.76 (m, 1H), 1.16-1.04 (m, 2H), 1.07-0.90 (m, 2H).

### [4-[1-cyclopropyl-4-(trifluoromethyl)imidazol-2-yl]phenyl]methanol

To a solution of 4-[1-cyclopropyl-4-(trifluoromethyl)imidazol-2-yl]benzoic acid (150 mg, 506.34 μmol) in tetrahydrofuran (3 mL) was added lithium aluminum hydride (38 mg, 1.0 mmol) at 0° C. The resulting solution was stirred at 15° C. for 16 h, then quenched by sodium sulfate decahydrate and stirred at 15° C. for 0.5 h. The resulted mixture was filtrated and the filtrate was concentrated under reduced pressure. The residue was purified by silica gel column chromatography, eluted with 25%-50% ethyl acetate in petroleum ether to afford [4-[1-cyclopropyl-4-(trifluoromethyl)imidazol-2-yl]phenyl]methanol (130 mg, 460.57 μmol, 90% yield) as a white solid. MS: m/z=283.00 [M+H]⁺. ¹H NMR (400 MHz, Methanol-d₄) δ 7.84-7.77 (m, 2H), 7.74 (s, 1H), 7.57-7.51 (m, 2H), 4.71 (s, 2H), 3.70-3.65 (m, 1H), 1.10-0.96 (m, 2H), 0.95-0.84 (m, 2H).

### 4-[1-cyclopropyl-4-(trifluoromethyl)imidazol-2-yl]benzaldehyde

To a solution of [4-[1-cyclopropyl-4-(trifluoromethyl)imidazol-2-yl]phenyl]methanol (130 mg, 460.57 μmol) in DCM (2 mL) was added manganese dioxide (400 mg, 4.6 mmol) at 15° C. The resulting solution was stirred at 40° C. for 16 h, then filtrated. The filtrate was concentrated under reduced pressure. The residue was purified by silica gel column chromatography, eluted with 15%-45% ethyl acetate in petroleum ether to afford 4-[1-cyclopropyl-4-(trifluoromethyl)imidazol-2-yl]benzaldehyde (120 mg, 428.20 μmol, 92% yield) as a yellow solid. MS: m/z=281.25 [M+H]⁺. ¹H NMR (400 MHz, Chloroform-d) δ 10.11 (s, 1H), 8.12-8.05 (m, 2H), 8.04-7.97 (m, 2H), 7.44 (s, 1H), 3.62-3.56 (m, 1H), 1.20-1.07 (m, 2H), 1.04-0.88 (m, 2H).

### [2-(4-cyclopropyl-6-methoxy-pyrimidin-5-yl)-5H-pyrrolo[3,2-d]pyrimidin-7-yl]-[4-[1-cyclopropyl-4-(trifluoromethyl)imidazol-2-yl]phenyl]methanol

To a solution of 2-(4-cyclopropyl-6-methoxy-pyrimidin-5-yl)-5H-pyrrolo[3,2-d]pyrimidine (114 mg, 428 μmol) in water (2 mL) and isopropyl alcohol (2 mL) was added 4-[1-cyclopropyl-4-(trifluoromethyl)imidazol-2-yl]benzaldehyde (120 mg, 428 μmol) at 15° C., then was stirred at 0° C. for 1 h. The resulting solution was stirred at 60° C. for 16 h, then diluted by ethyl acetate (30 mL), washed with saturated aqueous sodium chloride (10 mL×3). The organic layer was dried over anhydrous sodium sulfate. After filtration, the filtrate was concentrated under reduced pressure. The residue was purified by Prep-TLC (DCM:methanol=10:1) to give 31 mg crude product. The crude product was further purified by RP-Flash chromatography with the following conditions: Column: C18 spherical, 20-35 μm, 100A, 20 g; Mobile Phase A: Water (10 mM aq. NH₄HCO₂), Mobile Phase B: MeCN; Flow rate: 20 mL/min; Gradient: 5% B to 5% B in 5 min, 5% B to 20% B in 5 min, 20% B to 40% B in 20 min, 40% B to 95% B in 5 min; Detector: UV 254 & 220 nm; RT: 18 min. The collected fractions were combined, concentrated and then lyophilized overnight to give [2-(4-cyclopropyl-6-methoxy-pyrimidin-5-yl)-5H-pyrrolo[3,2-d]pyrimidin-7-yl]-[4-[1-cyclopropyl-4-(trifluoromethyl)imidazol-2-yl]phenyl]methanol (5.7 mg, 10.41 μmol, 2% yield) as a white solid. MS: m/z=548.30 [M+H]⁺. ¹H NMR (400 MHz, Chloroform-d) δ 10.94 (br., 1H), 8.96 (s, 1H), 8.67 (s, 1H), 7.80 (d, J=8.0 Hz, 2H), 7.65 (d, J=8.0 Hz, 2H), 7.41 (s, 1H), 7.17 (s, 1H), 6.42 (s, 1H), 4.46 (br., 1H), 3.94 (s, 3H), 3.58-3.50 (m, 1H), 1.84-1.76 (m, 1H), 1.28-1.23 (s, 2H), 1.14-1.06 (m, 2H), 0.97-0.97 (m, 4H).

### 2-(4-cyclopropyl-6-methoxy-pyrimidin-5-yl)-7-[[4-[1-cyclopropyl-4-(trifluoromethyl)imidazol-2-yl]phenyl]methyl]-5H-pyrrolo[3,2-d]pyrimidine

To a solution of [2-(4-cyclopropyl-6-methoxy-pyrimidin-5-yl)-5H-pyrrolo[3,2-d]pyrimidin-7-yl]-[4-[1-cyclopropyl-4-(trifluoromethyl)imidazol-2-yl]phenyl]methanol (120 mg, 219 μmol) in chloroform (2 mL) were added 2,2,2-trifluoroacetic acid (1 mL) and triethylsilane (2 mL) at 15° C., then stirred at 15° C. for 3 hr. The resulted solution was concentrated under reduced pressure. The residue was co-evaporated with toluene (three times, 2.5 mL each). The residue was purified by RP-Flash chromatography with the following conditions: Column: C18 spherical, 20-30 μm, 100A, 40 g; Mobile Phase A: Water (10 mM aq. NH₄HCO₃), Mobile Phase B: MeCN; Flow rate: 40 mL/min; Gradient: 5% B to 5% B in 5 min, 5% B to 25% B in 5 min, 25% B to 55% B in 30 min, 55% B to 95% B in 5 min; Detector: UV 254 & 220 nm; RT: 24 min. The collected fractions were combined, concentrated and then lyophilized overnight to give 2-(4-cyclopropyl-6-methoxy-pyrimidin-5-yl)-7-[[4-[l-cyclopropyl-4-(trifluoromethyl)imidazol-2-yl]phenyl]methyl]-5H-pyrrolo[3,2-d]pyrimidine (56.5 mg, 106.30 μmol, 48% yield) as a white solid. MS: m/z=532.30 [M+H]⁺. ¹H NMR (400 MHz, Chloroform-d) δ 10.61 (s, 1H), 8.92 (s, 1H), 8.65 (s, 1H), 7.74-7.67 (m, 2H), 7.44-7.36 (m, 3H), 7.13 (s, 1H), 4.28 (s, 2H), 3.91 (s, 3H), 3.54-3.48 (m, 1H), 1.73-1.67 (m, 1H), 1.24-1.16 (m, 2H), 1.13-1.03 (m, 2H), 0.95-0.87 (m, 2H), 0.87-0.79 (m, 2H).

### Example 19. Synthesis of 2-(2-isopropylphenyl)-7-[(1R)-1-[4-[1-methyl-4-(trifluoromethyl)imidazol-2-yl]phenyl]ethyl]-5H-pyrrolo[3,2-d]pyrimidine (Compound 64) & 2-(2-isopropylphenyl)-7-[(1S)-1-[4-[1-methyl-4-(trifluoromethyl)imidazol-2-yl]phenyl]ethyl]-5H-pyrrolo[3,2-d]pyrimidine (Compound 82)

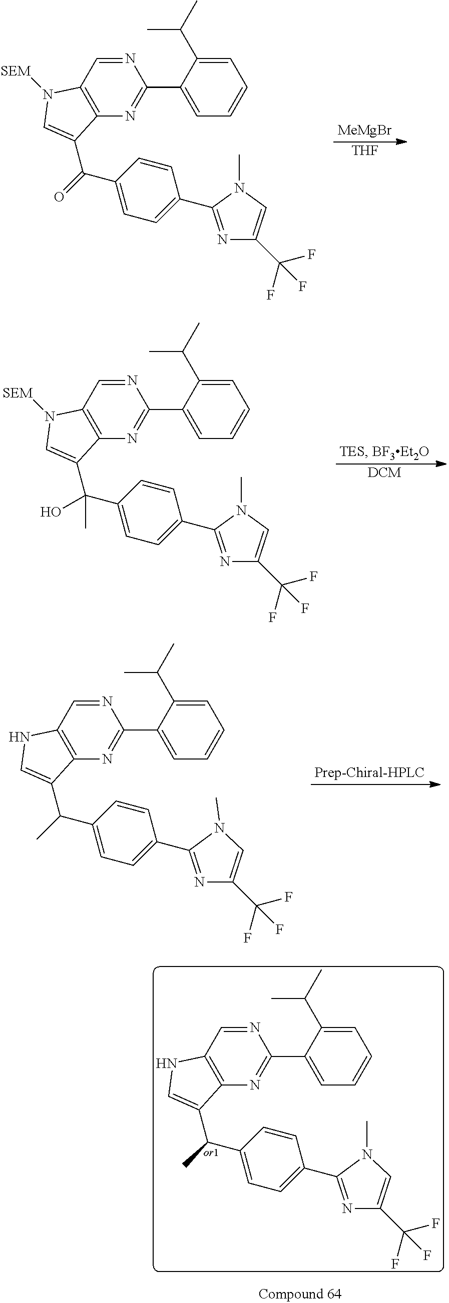

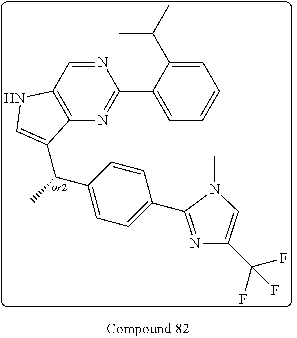 [SMILES: CC(C)c1ccccc1-c1ncc2[nH]cc([C@@H](C)c3ccc(-c4nc(C(F)(F)F)cn4C)cc3)c2n1]

### 1-[2-(2-isopropylphenyl)-5-(2-trimethylsilylethoxymethyl)pyrrolo[3,2-d]pyrimidin-7-yl]-1-[4-[1-methyl-4-(trifluoromethyl)imidazol-2-yl]phenyl]ethanol

A solution of [2-(2-isopropylphenyl)-5-(2-trimethylsilylethoxymethyl)pyrrolo[3,2-d]pyrimidin-7-yl]-[4-[1-methyl-4-(trifluoromethyl)imidazol-2-yl]phenyl]methanone (120 mg, 193.63 umol) in THF (2 mL) was added methyl magnesium bromide (1 M in THF, 580.9 μmol, 0.58 mL) at 0° C., then stirred at 25° C. for 1 hr under nitrogen atmosphere. The reaction mixture was quenched by saturated aqueous ammonium chloride (100 mL) and then extracted with ethyl acetate (3×50 mL). The combined organic layers were washed with brine (3×50 mL), dried over anhydrous sodium sulfate. After filtration, the filtrate was concentrated under reduced pressure. The residue was purified by Prep-TLC, eluted with 30% ethyl acetate in petroleum ether to give 1-[2-(2-isopropylphenyl)-5-(2-trimethylsilylethoxymethyl)pyrrolo[3,2-d]pyrimidin-7-yl]-1-[4-[1-methyl-4-(trifluoromethyl)imidazol-2-yl]phenyl]ethanol (90 mg, 141.56 μmol, 73% yield) as an off-white solid. MS: m/z=636.20 [M+H]⁺.

### 2-(2-isopropylphenyl)-7-[1-[4-[1-methyl-4-(trifluoromethyl)imidazol-2-yl]phenyl]ethyl]-5H-pyrrolo[3,2-d]pyrimidine

To a solution of 1-[2-(2-isopropylphenyl)-5-(2-trimethylsilylethoxymethyl)pyrrolo[3,2-d]pyrimidin-7-yl-]-1-[4-[1-methyl-4-(trifluoromethyl)imidazol-2-yl]phenyl]ethanol (90 mg, 141.56 μmol) and triethylsilane (329 mg, 2.8 mmol, 452 μL) in dichloromethane (1 mL) was added boron trifluoride ethyl ether (402 mg, 2.8 mmol, 356 μL) at 0° C. The resulting mixture was stirred at 25° C. for 16 h under nitrogen atmosphere. The resulting mixture was filtered and then concentrated under reduced pressure. The residue was purified by RP-Flash with the following conditions: Column: C18 spherical Column, 20-35 μm, 100A, 40 g; Mobile Phase A: Water, Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 33 min. The collected fractions were combined and concentrated under reduced pressure to give 2-(2-isopropylphenyl)-7-[1-[4-[1-methyl-4-(trifluoromethyl)imidazol-2-yl]phenyl]ethyl]-5H-pyrrolo[3,2-d]pyrimidine (50 mg, 102.14 μmol, 72% yield) as a brown oil. MS: m/z=490.25 [M+H]⁺.

### 2-(2-isopropylphenyl)-7-[(1R)-1-[4-[1-methyl-4-(trifluoromethyl)imidazol-2-yl]phenyl]ethyl]-5H-pyrrolo[3,2-d]pyrimidine (Compound 64) & 2-(2-isopropylphenyl)-7-[(1S)-1-[4-[1-methyl-4-(trifluoromethyl)imidazol-2-yl]phenyl]ethyl]-5H-pyrrolo[3,2-d]pyrimidine (Compound 82)

2-(2-isopropylphenyl)-7-[1-[4-[1-methyl-4-(trifluoromethyl)imidazol-2-yl]phenyl]ethyl]-5H-pyrrolo[3,2-d]pyrimidine (35 mg, 71.50 μmol) was separated by Prep-Chiral-HPLC with the following conditions: Column: CHIRALPAK IC, 2×25 cm, 5 μm; Mobile Phase A: Hex (0.2% DEA), Mobile Phase B: IPA; Flow rate: 20 mL/min; Gradient: 20% B to 20% B in 19 min; Detector: UV 254 & 210 nm; RT1; 11.365 min; RT2: 15.342 min; The first product containing (RT1: 11.365 min) peak was combined and concentrated under reduced pressure to give 2-(2-isopropylphenyl)-7-[(1R)-1-[4-[1-methyl-4-(trifluoromethyl)imidazol-2-yl]phenyl]ethyl]-5H-pyrrolo[3,2-d]pyrimidine (12.6 mg, 25.7 μmol, 36% yield, Compound 64) as an off-white solid. MS: m/z=490.15 [M+H]⁺. ¹H NMR (400 MHz, Chloroform-d) δ 10.14 (br., 1H), 8.92-8.86 (m, 1H), 7.61 (d, J=7.6 Hz, 1H), 7.58-7.52 (m, 2H), 7.50-7.41 (m, 4H), 7.35 (s, 1H), 7.32-7.24 (m, 1H), 7.19 (s, 1H), 4.73-4.63 (m, 1H), 3.77 (s, 3H), 3.54-3.43 (m, 1H), 1.78 (d, J=7.2 Hz, 3H), 1.22-1.15 (m, 6H). ¹⁹F NMR (376 MHz, Chloroform) δ −62.51.

The second product containing (RT2: 15.342 min) peak was combined and concentrated under reduced pressure to give 2-(2-isopropylphenyl)-7-[(1S)-1-[4-[1-methyl-4-(trifluoromethyl)imidazol-2-yl]phenyl]ethyl]-5H-pyrrolo[3,2-d]pyrimidine (8.5 mg, 17.36 μmol, 24% yield, Compound 82) as an off-white solid. MS: m/z=490.20 [M+H]⁺. ¹H NMR (400 MHz, Chloroform-d) δ 10.10-9.61 (m, 1H), 9.01-8.85 (m, 1H), 7.65-7.61 (m, 1H), 7.57-7.54 (m, 2H), 7.49-7.39 (m, 4H), 7.35 (s, 1H), 7.32-7.18 (m, 2H), 4.71-4.67 (m, 1H), 3.78 (s, 3H), 3.54-3.46 (m, 1H), 1.81-1.78 (m, 3H), 1.24-1.15 (m, 6H). ¹⁹F NMR (376 MHz, Chloroform-d) δ −62.517.

### Example 20. Synthesis of 1-[2-(2-isopropylphenyl)-5H-pyrrolo[3,2-d]pyrimidin-7-yl]-1-[4-[1-methyl-4-(trifluoromethyl)imidazol-2-yl]phenyl]ethanol (Compound 48)

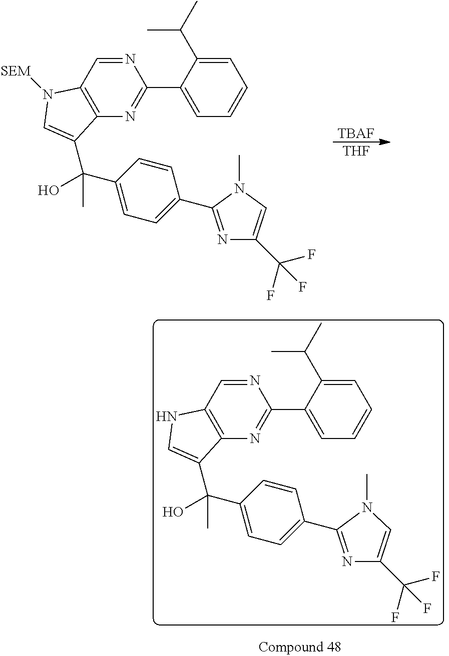

### 1-[2-(2-isopropylphenyl)-5H-pyrrolo[3,2-d]pyrimidin-7-yl]-1-[4-[1-methyl-4-(trifluoromethyl)imidazol-2-yl]phenyl]ethanol

A solution of 1-[2-(2-isopropylphenyl)-5-(2-trimethylsilylethoxymethyl)pyrrolo[3,2-d]pyrimidin-7-yl]-1-[4-[1-methyl-4-(trifluoromethyl)imidazol-2-yl]phenyl]ethanol (20 mg, 31 μmol) and tetrabutylammonium fluoride (33 mg, 125.8 μmol) in THF (I mL) was stirred at 60° C. for 4 hr under nitrogen atmosphere. The resulted reaction was concentrated under reduced pressure. The residue was purified by RP-Flash with the following conditions: Column: C18 spherical Column, 20-35 μm, 100A, 40 g; Mobile Phase A: Water, Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 36 min. The collected fractions were combined and concentrated under reduced pressure to afford 1-[2-(2-isopropylphenyl)-5H-pyrrolo[3,2-d]pyrimidin-7-yl]-1-[4-[1-methyl-4-(trifluoromethyl)imidazol-2-yl]phenyl]ethanol (5 mg, 9.89 μmol, 31% yield) as an off-white solid. MS: m/z=504.00 [M+H]⁺. ¹H NMR (400 MHz, Chloroform) δ 10.85-10.63 (m, 1H), 8.93-8.89 (m, 1H), 7.63-7.54 (m, 5H), 7.47-7.38 (m, 3H), 7.32-7.17 (m, 2H), 5.38 (br., 1H), 3.80 (s, 3H), 3.46-3.35 (m, 1H), 1.95 (d, J=6.0 Hz, 3H), 1.20-1.18 (m, 3H), 1.15-1.10 (m, 3H). ¹⁹F NMR (376 MHz, Chloroform-d) δ −62.39.

### Example 21. Synthesis of 5-(4-cyclopropyl-6-methoxy-pyrimidin-5-yl)-3-[[4-[1-methyl-4-(trifluoromethyl)imidazol-2-yl]phenyl]methyl]-1H-pyrrolo[2,3-c]pyridine (Compound 23)

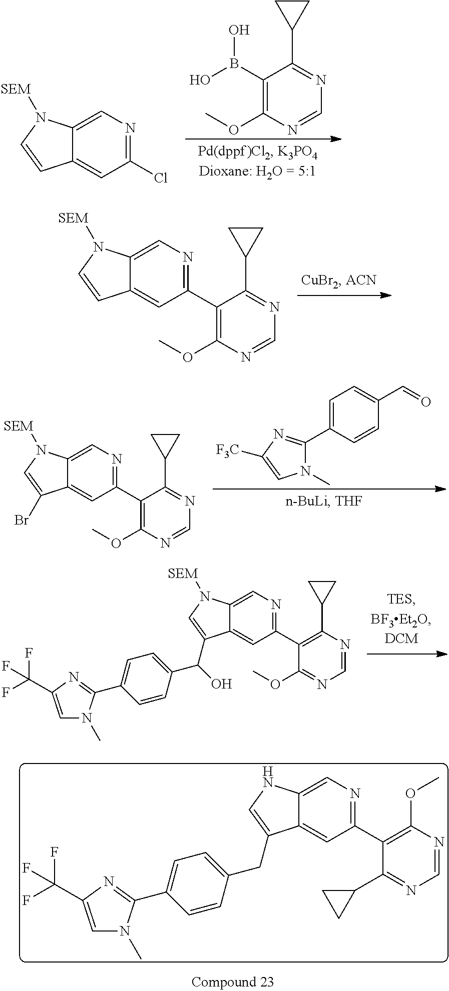

### 2-[[5-(4-cyclopropyl-6-methoxy-pyrimidin-5-yl)pyrrolo[2,3-c]pyridin-1-yl]methoxy]ethyl-trimethyl-silane

To a solution of 2-chloro-5-[[2-(trimethylsilyl)ethoxy]methyl]pyrrolo[3,2-d]pyrimidine (1 g, 3.04 mmol) and 4-cyclopropyl-6-methoxypyrimidin-5-ylboronic acid (709 mg, 3.6 mmol) in dioxane (10 mL) and water (2 mL) were added potassium phosphate tribasic (1.94 g, 9.13 mmol) and 1,1′-Bis(diphenylphosphino)ferrocene-palladium(II)dichloride dichloromethane complex (248 mg, 0.30 mmol) under nitrogen atmosphere. After stirring for 16 h at 100° C. under nitrogen atmosphere, the resulting mixture was concentrated under reduced pressure. The residue was purified by RP-Flash with the following conditions: Column: Spherical C18, 20-40 um, 40 g; Mobile Phase A: Water (10 mM aq. NH₄HCO₃), Mobile Phase B: MeCN; Flow rate: 40 mL/min; Gradient (B %): 0% hold 5 min, 5% B to 80% B in 10 min; 80% hold 5 min, 80% B to 95% B in 20 min, 100% hold 3 min; Detector: UV 254 & 220 nm; RT: 17 min. The collected fractions were combined and concentrated under reduced pressure to afford 2-[[5-(4-cyclopropyl-6-methoxy-pyrimidin-5-yl)pyrrolo[2,3-c]pyridin-1-yl]methoxy]ethyl-trimethyl-silane (0.67 g, 1.69 mmol, 55% yield) as a dark yellow solid. MS: m/z=397.20 [M+H]⁺. ¹H NMR (400 MHz, Chloroform-d) δ 9.05 (s, 1H), 8.65 (s, 1H), 7.65 (s, 1H), 7.40 (d, J=3.2 Hz, 1H), 6.62 (d, J=3.2 Hz, 1H), 5.61 (s, 2H), 3.93 (s, 3H), 3.61-3.47 (m, 2H), 1.85-1.75 (m, 1H), 1.25-1.16 (m, 2H), 1.01-0.89 (m, 2H), 0.94-0.83 (m, 2H), −0.02 (s, 9H).

### 5-(3-bromo-1-[[2-(trimethylsilyl)ethoxy]methyl]pyrrolo[2,3-c]pyridin-5-yl)-4-cyclopropyl-6-methoxypyrimidine

To a stirring solution of 4-cyclopropyl-6-methoxy-5-(1-[[2-(trimethylsilyl)ethoxy]methyl]pyrrolo[2,3-c]pyridin-5-yl)pyrimidine (670 mg, 1.69 mmol) in acetonitrile (7 mL) was added cupric bromide (1.13 g, 5.07 mmol) at room temperature. The resulting mixture was stirred for 16 h at room temperature, then the ammonia (7.0 M in methanol, 2 mL) was added at room temperature. The resulting mixture was stirred for additional 30 min at room temperature then diluted with ethyl acetate (50 mL), washed with brine (3×30 mL), dried over anhydrous sodium sulfate. After filtration, the filtrate was concentrated under reduced pressure. The residue was purified by silica gel column chromatography, eluted with 1˜50% ethyl acetate in petroleum ether to afford 5-(3-bromo-1-[[2-(trimethylsilyl)ethoxy]methyl]pyrrolo[2,3-c]pyridin-5-yl)-4-cyclopropyl-6-methoxypyrimidine (670 mg, 1.40 mmol, 83%) as a light yellow oil. MS: m/z=475.15, 477.15 [M+H]⁺. ¹H NMR (400 MHz, Chloroform-d) δ 9.04 (s, 1H), 8.67 (s, 1H), 7.61 (s, 1H), 7.44 (s, 1H), 5.59 (s, 2H), 3.94 (s, 3H), 3.62-3.49 (m, 2H), 1.80-1.70 (m, 1H), 1.32-1.18 (m, 2H), 1.01-0.85 (m, 4H), −0.00 (s, 9H).

### [5-(4-cyclopropyl-6-methoxypyrimidin-5-yl)-1-[[2-(trimethylsilyl)ethoxy]methyl]pyrrolo[2,3-c]pyridin-3-yl]([4-[1-methyl-4-(trifluoromethyl)imidazol-2-yl]phenyl])methanol

To a solution of 5-(3-bromo-1-[[2-(trimethylsilyl)ethoxy]methyl]pyrrolo[2,3-c]pyridin-5-yl)-4-cyclopropyl-6-methoxypyrimidine (200 mg, 0.42 mmol) and 4-[1-methyl-4-(trifluoromethyl)imidazol-2-yl]benzaldehyde (107 mg, 0.42 mmol) in THF (2 mL), n-butyllithium solution (2.5 M in hexane, 0.20 mL, 0.51 mmol) was added dropwise at −70° C. under nitrogen atmosphere. The reaction mixture was warmed up to room temperature and stirred for 1 h and then quenched with saturated aqueous ammonium chloride (50 mL). The resulting mixture was extracted with ethyl acetate (60 mL×2). The combined organic layers were washed with brine (3×30 mL), dried over anhydrous sodium sulfate. After filtration, the filtrate was concentrated under reduced pressure. The residue was purified by silica gel column chromatography, eluted with 1˜10% methanol in dichloromethane to afford [5-(4-cyclopropyl-6-methoxypyrimidin-5-yl)-1-[[2-(trimethylsilyl)ethoxy]methyl]pyrrolo[2,3-c]pyridin-3-yl]([4-[1-methyl-4-(trifluoromethyl)imidazol-2-yl]phenyl])methanol (170 mg, 0.26 mmol, 62%) as a brown solid. MS: m/z=651.15 [M+H]⁺. ¹H NMR (400 MHz, Chloroform-d) δ 9.08 (s, 1H), 8.65 (s, 1H), 7.77-7.57 (m, 5H), 7.35 (s, 1H), 7.15 (s, 1H), 6.25 (s, 1H), 5.54 (s, 2H), 3.93 (s, 3H), 3.80 (s, 3H), 3.60-3.53 (m, 2H), 1.81-1.70 (m, 1H), 1.24-1.17 (m, 2H), 0.99-0.93 (m, 2H), 0.90-0.84 (m, 2H), −0.01 (s, 9H).

### 5-(4-cyclopropyl-6-methoxy-pyrimidin-5-yl)-3-[[4-[1-methyl-4-(trifluoromethyl)imidazol-2-yl]phenyl]methyl]-1H-pyrrolo[2,3-c]pyridine

To a stirred solution of [5-(4-cyclopropyl-6-methoxypyrimidin-5-yl)-1-[[2-(trimethylsilyl)ethoxy]methyl]pyrrolo[2,3-c]pyridin-3-yl]([4-[1-methyl-4-(trifluoromethyl)imidazol-2-yl]phenyl])methanol (30 mg, 0.05 mmol) in DCM (1.00 mL) was added triethylsilane (107 mg, 0.9 mmol) dropwise at 0° C. under nitrogen atmosphere. Five minutes later, to the above mixture was added boron trifluoride etherate (131 mg, 0.92 mmol) dropwise at 0° C. The resulting mixture was stirred at 0° C. for 16 h and then concentrated under reduced pressure. The residue was purified by RP-Flash with the following conditions: Column: Spherical C18, 20-40 um, 40 g; Mobile Phase A: Water (10 mM aq. NH₄HCO₃), Mobile Phase B: MeCN; Flow rate: 40 mL/min; Gradient (B %): 5% B hold 5 min, 5% B to 60% B in 25 min, 60% B hold 3 min, 60% B to 95% B in 2 min, 95% B hold 5 min; Detector: UV 254 & 220 nm; RT: 31 min. The collected fractions were combined and concentrated under reduced pressure to afford 5-(4-cyclopropyl-6-methoxy-pyrimidin-5-yl)-3-[[4-[1-methyl-4-(trifluoromethyl)imidazol-2-yl]phenyl]methyl]-1H-pyrrolo[2,3-c]pyridine (10.20 mg, 20.22 μmol, 44% yield) as an off-white solid. MS: m/z=505.15 [M+H]⁺. ¹H NMR (400 MHz, DMSO-d₆) δ 11.53 (s, 1H), 8.80 (s, 1H), 8.60 (s, 1H), 7.91 (s, 1H), 7.62 (d, J=8.0 Hz, 2H), 7.58-7.52 (m, 2H), 7.45 (d, J=8.4 Hz, 2H), 4.15 (s, 2H), 3.78 (s, 3H), 3.74 (s, 3H), 1.78-1.69 (m, 1H), 1.02-0.95 (m, 2H), 0.83-0.73 (m, 2H). ¹⁹F NMR (376 MHz, DMSO-d₆) δ −63.93

### Example 22. Synthesis of [2-(4-cyclopropyl-6-methoxy-pyrimidin-5-yl)-5H-pyrrolo[3,2-d]pyrimidin-7-yl]-[4-[1-isopropyl-4-(trifluoromethyl)imidazol-2-yl]phenyl]methanol (Compound 59) and 2-(4-cyclopropyl-6-methoxy-pyrimidin-5-yl)-7-[[4-[1-isopropyl-4-(trifluoromethyl)imidazol-2-yl]phenyl]methyl]-5H-pyrrolo[3,2-d]pyrimidine (Compound 54)

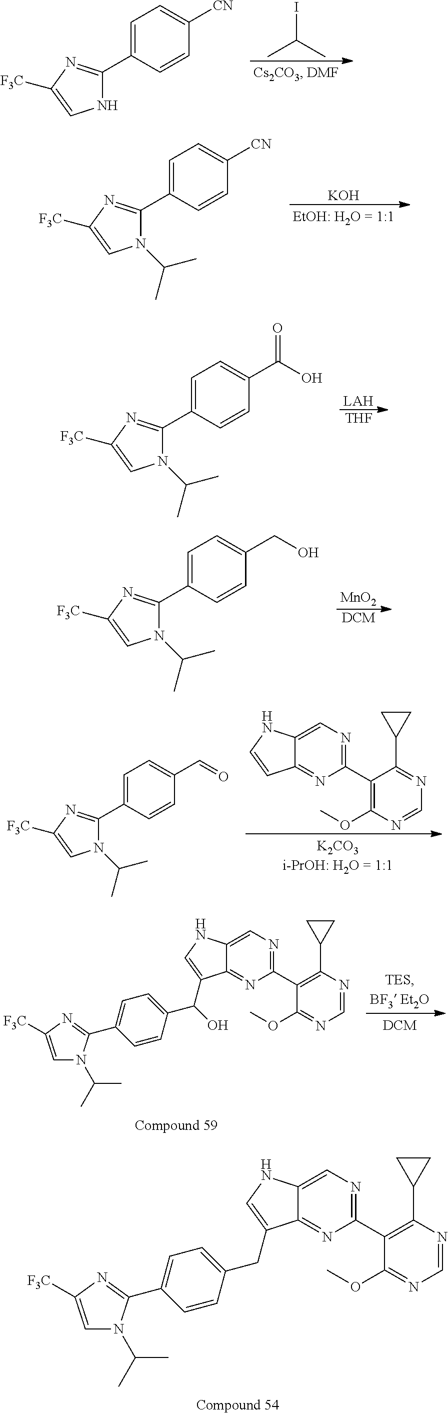

### 4-[1-isopropyl-4-(trifluoromethyl)imidazol-2-yl]benzonitrile

To a solution of 4-[4-(trifluoromethyl)-1H-imidazol-2-yl]benzonitrile (2.96 g, 12.48 mmol) in DMF (15 mL) were added 2-iodopropane (2.55 g, 14.98 mmol) and cesium carbonate (4.88 g, 14.98 mmol) at 25° C. The resulting mixture was warmed up to 110° C., and stirred for 18 h then naturally cooled down to 25° C. The resulted mixture was diluted by EA (100 mL×3). The organic layer was washed with brine (100 mL×3), dried over anhydrous sodium sulfate. After filtration, the filtrate was concentrated under reduced pressure. The residue was purified by silica gel column chromatography, eluted with 0%-25% EtOAc in PE to give 4-[1-isopropyl-4-(trifluoromethyl)imidazol-2-yl]benzonitrile (1.06 g, 3.80 mmol, 30% yield) as a yellow solid. MS: m/z=280.15 [M+H]⁺. ¹H NMR (400 MHz, Chloroform-d) δ 7.85-7.77 (m, 2H), 7.75-7.68 (m, 2H), 7.50 (s, 1H), 4.60-4.54 (m, 1H), 1.51 (d, J=6.8 Hz, 6H).

### 4-[-isopropyl-4-(trifluoromethyl)imidazol-2-yl]benzoic Acid

To a solution of 4-[1-isopropyl-4-(trifluoromethyl)imidazol-2-yl]benzonitrile (1.06 g, 3.80 mmol) in H₂O (10 mL) and EtOH (10 mL) was added potassium hydroxide (1.06 g, 18.98 mmol) at 25° C. After the resulting solution was stirred for 3 hours at 90° C., it was naturally cooled down to 25° C. The reaction mixture was adjusted pH to 5 with 1 M aq. HCl and then purified by RP-Flash chromatography with the following conditions: Column: C18 spherical, 20-35 um, 100A, 330 g; Mobile Phase A: Water, Mobile Phase B: MeCN; Flow rate: 80 mL/min; Gradient: 5% B to 5% B in 5 min, 5% B to 45% B in 20 min; 45% B to 45% B in 5 min; 45% B to 95% B in 5 min; Detector: UV 254 & 220 nm; RT: 30 min to give 4-[1-isopropyl-4-(trifluoromethyl)imidazol-2-yl]benzoic acid (0.81 g, 2.72 mmol, 71% yield) as an off-white solid. MS: m/z=299.15 [M+H]⁺. ¹H NMR (400 MHz, Chloroform-d) δ 8.24 (d, J=8.0 Hz, 2H), 7.70 (d, J=8.0 Hz, 2H), 7.49 (s, 1H), 4.65-4.58 (m, 1H), 1.51 (d, J=6.8 Hz, 6H).

### (4-(1-isopropyl-4-(trifluoromethyl)-1H-imidazol-2-yl)phenyl)methanol

To a solution of 4-[1-isopropyl-4-(trifluoromethyl)imidazol-2-yl]benzoic acid (680 mg, 2.28 mmol) in THF (8 mL) was added lithium aluminum hydride (113 mg, 2.9 mmol) in several portions at 0° C. The resulting mixture stirred for 1 hour under N₂ atmosphere, then was warmed to room temperature and stirred for 4 hours under N₂ atmosphere. The resulted mixture was quenched by saturated aqueous ammonium chloride (30 mL), was extracted by EA (50 mL×2). The organic layer was washed with brine (30 mL×2), dried over anhydrous sodium sulfate. After filtration, the filtrate was concentrated under reduced pressure, which was used directly in the next step without further purification. MS: m/z=285.00 [M+H]⁺.

### 4-[1-isopropyl-4-(trifluoromethyl)imidazol-2-yl]benzaldehyde

To a solution of [4-[1-isopropyl-4-(trifluoromethyl)imidazol-2-yl]phenyl]methanol (588 mg, 2.07 mmol) in DCM (15 mL) was added manganese(IV) oxide (1.80 g, 20.68 mmol) at 25° C. The resulting mixture was stirred for 16 hours at 40° C. then naturally cooled down to 25° C. The resulted mixture was filtered through a Celite pad and the filtrate was concentrated under reduced pressure. The residue was purified by silica gel column chromatography, eluted with 0%-10% EtOAc in PE to afford 4-[1-isopropyl-4-(trifluoromethyl)imidazol-2-yl]benzaldehyde (580 mg, 2.05 mmol, 99% yield) as an off-white solid. MS: m/z=283.20 [M+H]⁺. ¹H NMR (400 MHz, Chloroform-d) δ 10.12 (s, 1H), 8.06-7.99 (m, 2H), 7.79-7.77 (m, 2H), 7.50 (s, 1H), 4.65-4.62 (m, J=6.8 Hz, 1H), 1.52 (d, J=6.8 Hz, 6H).

### [2-(4-cyclopropyl-6-methoxy-pyrimidin-5-yl)-5H-pyrrolo[3,2-d]pyrimidin-7-yl]-[4-[1-isopropyl-4-(trifluoromethyl)imidazol-2-yl]phenyl]methanol

To a solution of 4-[1-isopropyl-4-(trifluoromethyl)imidazol-2-yl]benzaldehyde (579 mg, 2.05 mmol) and 2-(4-cyclopropyl-6-methoxy-pyrimidin-5-yl)-5H-pyrrolo[3,2-d]pyrimidine (548 mg, 2.05 mmol) in i-PrOH (5 mL) and H₂O (5 mL) was added potassium carbonate (340 mg, 2.46 mmol). The resulting solution was stirred for 16 h at 60° C. then naturally cooled down to 25° C. The reaction was diluted by H₂O (50 mL). The mixture solution was extracted by EA (50 mL×3). The organic layer was washed with brine (50 mL×3), then dried over anhydrous sodium sulfate. After filtration, the filtrate was concentrated under reduced pressure. The residue was purified by silica gel column chromatography, eluted with 0%-30% EtOAc in PE to afford a crude product, then the crude product was further purified by RP-Flash chromatography with the following conditions: Column: C18 spherical, 20˜30 um, 100A, 40 g; Mobile Phase A: 10 mM aq. NH₄HCO₃, Mobile Phase B: MeCN; Flow rate: 50 mL/min; Gradient: 5% B to 5% B in 5 min, 5% B to 35% B in 15 min; 35% B to 35% B in 5 min; 35% B to 95% B in 5 min; Detector: UV 254 & 220 nm; RT: 20 min to give [2-(4-cyclopropyl-6-methoxy-pyrimidin-5-yl)-5H-pyrrolo[3,2-d]pyrimidin-7-yl]-[4-[1-isopropyl-4-(trifluoromethyl)imidazol-2-yl]phenyl]methanol (128 mg, 232.92 μmol, 11% yield) as an off-white solid. MS: m/z=550.20 [M+H]⁺; ¹H NMR (400 MHz, Chloroform-d) δ 9.02 (s, 1H), 8.69 (s, 1H), 7.69 (d, J=8.4 Hz, 2H), 7.56 (d, J=8.4 Hz, 2H), 7.47 (s, 1H), 6.43 (s, 1H), 4.61-4.54 (m, 1H), 3.97 (s, 3H), 1.89-1.79 (m, 6H), 1.52 (d, J=6.8 Hz, 2H), 1.35-1.28 (m, 2H), 0.95-0.86 (m, 2H). ¹⁹F NMR (376 MHz, Chloroform-d) δ −62.48.

### 2-(4-cyclopropyl-6-methoxy-pyrimidin-5-yl)-7-[[4-[1-isopropyl-4-(trifluormethyl)imidazol-2-yl]phenyl]methyl]-5H-pyrrolo[3,2-d]pyrimidine

To a solution of [2-(4-cyclopropyl-6-methoxy-pyrimidin-5-yl)-5H-pyrrolo[3,2-d]pyrimidin-7-yl]-[4-[1-isopropyl-4-(trifluoromethyl)imidazol-2-yl]phenyl]methanol (100 mg, 181.97 umol) in DCM (5 mL) was added triethylsilane (423 mg, 3.6 mmol) and boron trifluoride etherate (517 mg, 3.6 mmol) under N₂ at 0° C. The resulting mixture was warmed to 30° C., and stirred for 16 h. The resulted mixture was quenched by saturated aqueous ammonium chloride (30 mL), was extracted by EA (30 mL×3). The organic layer was washed with brine (30 mL×3), dried over anhydrous sodium sulfate. After filtration, the filtrate was concentrated under reduced pressure. The residue was purified by Prep-TLC (DCM:MeOH=20: 1) to give a crude product. The crude product was further purified by RP-Flash chromatography with the following conditions: Column: C18 spherical, 20-30 um, 100A, 40 g; Mobile Phase A: 10 mM aq. NH₄HCO₁, Mobile Phase B: MeCN; Flow rate: 50 mL/min; Gradient: 5% B to 5% B in 5 min, 5% B to 55% B in 15 min; 55% B to 55% B in 5 min; 55% B to 95% B in 5 min; Detector: UV 254 & 220 nm; RT: 20 min to give 2-(4-cyclopropyl-6-methoxy-pyrimidin-5-yl)-7-[[4-[I-isopropyl-4-(trifluoromethyl)imidazol-2-yl]phenyl]methyl]-5H-pyrrolo[3,2-d]pyrimidine (46.4 mg, 86.97 μmol, 47% yield) as an off-white solid. MS: m/z=534.20 [M+H]⁺; ¹H NMR (400 MHz, Chloroform-d) δ 10.77 (s, 1H), 8.95 (s, 1H), 8.66 (d, J=4.4 Hz, 1H), 7.48-7.29 (m, 5H), 7.04 (d, J=2.0 Hz, 1H), 4.60-4.53 (m, 1H), 4.28 (s, 2H), 3.92 (s, 3H), 1.74-1.67 (m, 1H), 1.48 (d, J=6.8 Hz, 6H), 1.23-1.19 (m, 2H), 0.84-0.83 (m, 2H); ¹⁹F NMR (376 MHz, Chloroform-d) δ −62.28, −62.32, −62.36.

### Example 23. Synthesis of [5-(2-cyclopropylpyridin-3-yl)-1H-pyrrolo[2,3-c]pyridin-3-yl([4-[1-methyl-4-(trifluoromethyl)imidazol-2-yl]phenyl])methanol (Compound 30) and 2-cyclopropyl-3-[3-([4-[1-methyl-4-(trifluoromethyl)imidazol-2-yl]phenyl]methyl)-1H-pyrrolo[2,3-c]pyridin-5-yl]pyridine (Compound 66)

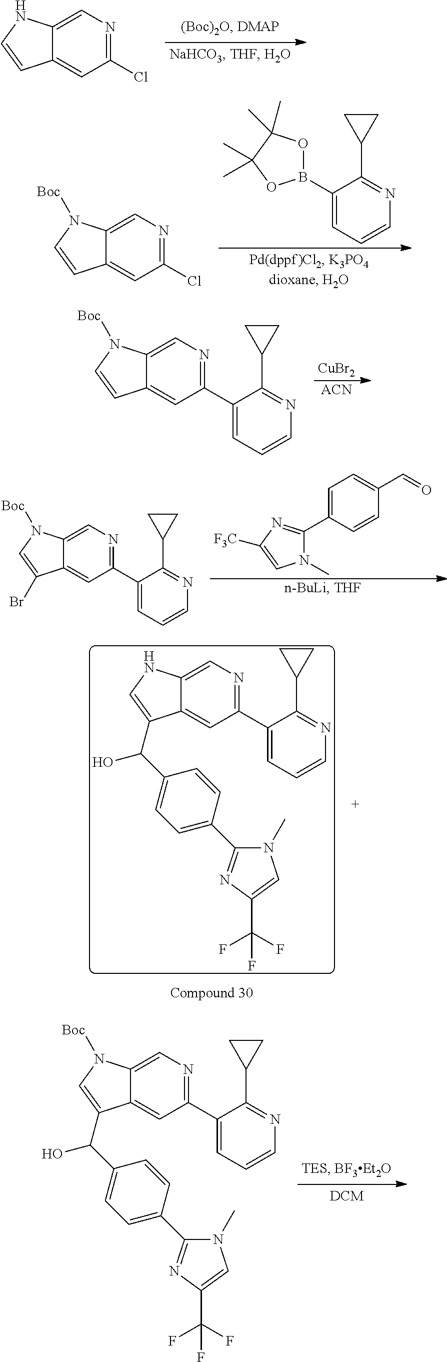

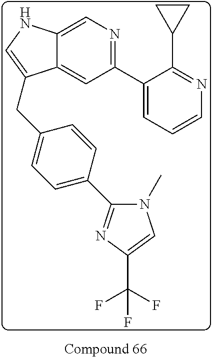 [SMILES: Cn1cc(C(F)(F)F)nc1-c1ccc(Cc2c[nH]c3cnc(-c4cccnc4C4CC4)cc23)cc1]

### tert-butyl 5-chloropyrrolo[2,3-c]pyridine-1-carboxylate

To a stirred mixture of 5-chloro-1H-pyrrolo[2,3-c]pyridine (1.00 g, 6.55 mmol) and Boc₂O (5.72 g, 26.21 mmol) in THF (10 mL) and H₂O (10 mL) were added NaHCO₃ (2.20 g, 26.21 mmol) and DMAP (80 mg, 0.65 mmol) at room temperature under Ar atmosphere. After the resulting solution was stirred at room temperature for 16 h, it was extracted with EtOAc (3×10 mL). The combined organic layers were washed with brine (3×10 mL), dried over anhydrous Na₂SO₄. The filtrate was concentrated under reduced pressure. The residue was purified by silica gel column chromatography, eluted with PE:EtOAc=2: 1 to afford tert-butyl 5-chloropyrrolo[2,3-c]pyridine-1-carboxylate as a white solid (1.50 g, 5.94 mmol, 90% yield). MS: m/z=253.05 [M+H]⁺.

### tert-butyl 5-(2-cyclopropyl-3-pyridyl)pyrrolo[2,3-c]pyridine-1-carboxylate

To a solution of tert-butyl 5-chloropyrrolo[2,3-c]pyridine-1-carboxylate (500.00 mg, 1.97 mmol) and 2-cyclopropyl-3-(4,4,5,5-tetramethyl-1,3,2-dioxaborolan-2-yl)pyridine (534 mg, 2.2 mmol) in dioxane (5 mL) and H₂O (1 mL) were added [1,1′-Bis(diphenylphosphino)ferrocene]dichloropalladium(II)(162 mg, 0.197 mmol) and K₃PO₄ (1.26 g, 5.93 mmol). After stirring for 5 h at 100° C. under nitrogen atmosphere, the resulting mixture was concentrated under reduced pressure. The residue was purified by silica gel column chromatography, eluted with PE:EtOAc=2:1 to afford tert-butyl 5-(2-cyclopropylpyridin-3-yl)pyrrolo[2,3-c]pyridine-1-carboxylate (376 mg, 1.12 mmol, 57%) as a light yellow oil. MS: m/z=336.15 [M+H]⁺.

### tert-butyl 3-bromo-5-(2-cyclopropyl-3-pyridyl)pyrrolo[2,3-c]pyridine-1-carboxylate

To a stirred solution of tert-butyl 5-(2-cyclopropylpyridin-3-yl)pyrrolo[2,3-c]pyridine-1-carboxylate (500 mg, 1.49 mmol) in MeCN (10 mL) was added cupric bromide (2 g, 8.94 mmol) at room temperature. After the resulting mixture was stirred for 16 h at room temperature. The reaction was quenched with 7 M NH₃ in MeOH at room temperature and stirred for 30 mins. The resulted mixture was diluted by saturated sodium bicarbonate solution (100 mL), extracted with EtOAc (3×100 mL). The combined organic layers were washed with brine (3×100 mL), dried over anhydrous Na₂SO₄. The filtrate was concentrated under reduced pressure. The residue was purified by Prep-TLC (PE:EtOAc=2: 1) to afford tert-butyl 3-bromo-5-(2-cyclopropylpyridin-3-yl)pyrrolo[2,3-c]pyridine-1-carboxylate (376 mg, 0.91 mmol, 61%) as a light yellow oil. MS: m/z=414.00, 416.00 [M+H]⁺.

### tert-butyl 5-(2-cyclopropyl-3-pyridyl)-3-[hydroxy-[4-[1-methyl-4-(trifluoromethyl)imidazol-2-yl]phenyl]methyl]pyrrolo[2,3-c]pyridine-1-carboxylate

To a solution of tert-butyl 3-bromo-5-(2-cyclopropylpyridin-3-yl)pyrrolo[2,3-c]pyridine-1-carboxylate (40 mg, 0.09 mmol) in THF (1 mL), n-butyllithium (2.5 M in hexane, 0.048 mL, 0.12 mmol) was added dropwise at −70° C. under N₂ atmosphere. The reaction mixture was stirred at −70° C. for 30 mins, then a solution of 4-[1-methyl-4-(trifluoromethyl)imidazol-2-yl]benzaldehyde (30.48 mg, 0.12 mmol) in 0.3 mL THF was added dropwise. The resulting mixture was stirred at −70° C. for another 2 h then quenched with saturated aqueous ammonium chloride (20 mL) and extracted with EtOAc (3×20 mL). The combined organic phase was washed with brine (30 mL), dried over anhydrous Na₂SO₄. The filtrate was concentrated under vacuum to give two desired crude product. The crude residue was purified by reverse flash chromatography with the following conditions: Column: YMC-Actus Triart C18, 20×250 mm, 5 μm, 12 nm; Mobile Phase A: 5 mM aq. NH₄HCO₃, Mobile Phase B: ACN; Flow rate: 60 mL/min; Gradient: 30% B to 55% B in 8 min; Detector: UV 254 & 210 nm; RT1: 6.12 min; RT2: 7.52 min. The fractions (RT1: 6.12 min) were collected and concentrated under reduced pressure and then lyophilized overnight to afford [5-(2-cyclopropylpyridin-3-yl)-1H-pyrrolo[2,3-c]pyridin-3-yl]([4-[1-methyl-4-(trifluoromethyl)imidazol-2-yl]phenyl])methanol (1.3 mg, 2.65 μmol, 3%) as a white solid. MS: m/z=490.10 [M+H]⁺. ¹H NMR (400 MHz, Methanol-d₄) δ 8.78 (s, 1H), 8.40 (d, J=3.6 Hz, 1H), 7.74-7.63 (m, 6H), 7.55 (s, 1H), 7.27-7.20 (m, 1H), 6.24 (s, 1H), 4.60 (s, 1H), 3.77 (s, 3H), 2.11-1.98 (m, 1H), 1.08-1.00 (m, 2H), 0.87-0.78 (m, 2H). ¹⁹F NMR (376 MHz, Methanol-d₄) δ −60.82.

The fractions (RT2: 7.52 min) were collected and concentrated under reduced pressure and then lyophilized overnight to afford 5-(2-cyclopropyl-3-pyridyl)-3-[hydroxy-[4-[1-methyl-4-(trifluoromethyl)imidazol-2-yl]phenyl]methyl]pyrrolo[2,3-c]pyridine-1-carboxylate (10 mg, 0.017 mmol) as a white solid. MS: m/z=590.40 [M+H]⁺.

### 5-(2-cyclopropyl-3-pyridyl)-3-[[4-[1-methyl-4-(trifluoromethyl)imidazol-2-yl]phenyl]methyl]-1H-pyrrolo[2,3-c]pyridine

To a stirred solution of tert-butyl 5-(2-cyclopropylpyridin-3-yl)-3-[hydroxy([4-[1-methyl-4-(trifluoromethyl)imidazol-2-yl]phenyl])methyl]pyrrolo[2,3-c]pyridine-1-carboxylate (60.00 mg, 0.10 mmol) in DCM (2 mL) were added triethylsilane (237 mg, 2.0 mmol) and boron trifluoride etherate (289 mg, 2.0 mmol) dropwise at 0° C. The resulting mixture was stirred for 1 h at 0° C. then warmed to room temperature and stirred for 16 h. The resulting mixture was diluted with EtOAc (100 mL). The combined organic layers was washed with saturated sodium bicarbonate solution (3×20 mL), dried over anhydrous Na₂SO₄. The filtrate was concentrated under reduced pressure. The residue was purified by reverse phase chromatography with the following conditions: Column: Spherical C18, 20-40 μm, 40 g; Mobile Phase A: 10 mM aq. NH₄HCO₃, Mobile Phase B: ACN; Flow rate: 40 mL/min; Gradient (B %): 0% hold 5 min, 5%-20% within 5 min; 20% hold 5 min, 20%-40% within 20 min, 40% hold 3 min, 40%-95% within 2 min, 95% hold 5 min; Detector: UV 254 & 220 nm; RT: 34 min. The collected fractions were combined and concentrated under reduced pressure to afford 2-cyclopropyl-3-[3-([4-[1-methyl-4-(trifluoromethyl)imidazol-2-yl]phenyl]methyl)-1H-pyrrolo[2,3-c]pyridin-5-yl]pyridine as a white solid. MS: m/z=474.15 [M+H]⁺. ¹H NMR (400 MHz, Chloroform-d) δ 9.24 (br., 1H), 9.02 (s, 1H), 8.47 (d, J=4.8 Hz, 1H), 7.75-7.65 (m, 2H), 7.62-7.54 (m, 2H), 7.42-7.35 (m, 2H), 7.35-7.30 (m, 1H), 7.27 (s, 1H), 7.13 (dd, J=7.6, 4.8 Hz, 1H), 4.21 (s, 2H), 3.77 (s, 3H), 2.21-2.15 (m, 1H), 1.18-1.14 (m, 2H), 0.84-0.80 (m, 2H). ¹⁹F NMR (376 MHz, Chloroform-d) δ −62.72.

### Example 24. Synthesis of 2-(2-cyclopropyl-3-pyridyl)-7-[[4-[1-methyl-4-(trifluoromethyl)imidazol-2-yl]phenyl]methyl]-5H-pyrrolo[3,2-d]pyrimidine (Compound 60)

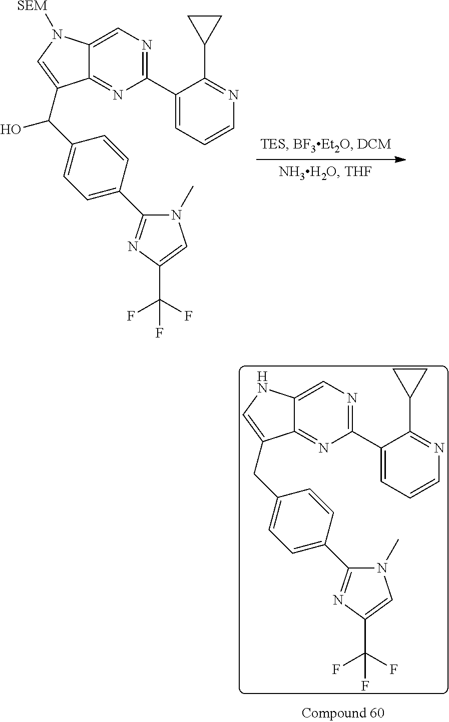 [SMILES: Cn1cc(C(F)(F)F)nc1-c1ccc(C(O)c2cn(COCC[Si](C)(C)C)c3cnc(-c4cccnc4C4CC4)nc23)cc1 ; Cn1cc(C(F)(F)F)nc1-c1ccc(Cc2c[nH]c3cnc(-c4cccnc4C4CC4)nc23)cc1]

### 2-(2-cyclopropyl-3-pyridyl)-7-[[4-[1-methyl-4-(trifluoromethyl)imidazol-2-yl]phenyl]methyl]-5H-pyrrolo[3,2-d]pyrimidine

To a solution of [2-(2-cyclopropyl-3-pyridyl)-5-(2-trimethylsilylethoxymethyl) pyrrolo[3,2-d]pyrimidin-7-yl]-[4-[1-methyl-4-(trifluoromethyl)imidazol-2-yl] phenyl]methanol (19 mg, 31 umol) in dry DCM (1 mL) was added triethylsilane (71 mg, 612 μmol, 98 μL) at 0° C. under N₂ atmosphere. After the reaction mixture was stirred at room temperature for 50 min, boron trifluoride etherate (87 mg, 612 μmol) was added at 0° C. After the reaction mixture was stirred at room temperature for 3 hr, it was concentrated under vacuum. To the residue, ammonium hydroxide (28% solution, 2 mL) was added and the resulting mixture was stirred for 30 minutes. The reaction was quenched with brine (50 mL) and the mixture solution was extracted with EA (3×100 mL). The combine organic was dried over anhydrous Na₂SO₄. After filtration, the filtrate was concentrated under reduced pressure to yield a crude product. The residue was purified by RP-Flash chromatography with the following conditions: Column: C18 gel column (40 g), 20-35 μm, 19×150 mm; mobile phase: acetonitrile and 5 mM aq. Ammonium hydrogen carbonate (Gradient: 45% hold 10 min, up to 60% within 20 min; Flow rate: 50 mL/min; Detector: UV 254 & 210 nm; RT: 18 min; The fractions were collected and concentrated under reduced pressure and then lyophilized overnight to give 2-(2-cyclopropyl-3-pyridyl)-7-[[4-[1-methyl-4-(trifluoromethyl)imidazol-2-yl]phenyl]methyl]-5H-pyrrolo[3,2-d]pyrimidine (13 mg, 27.40 umol, 89% yield) as a white solid. MS: m/z=475.30 [M+H]⁺. ¹H NMR (400 MHz, DMSO-d₆) δ 11.76 (d, J=2.4 Hz, 1H), 9.03 (s, 1H), 8.45 (dd, J=4.8, 1.6 Hz, 1H), 8.03 (dd, J=8.0, 2.0 Hz, 1H), 7.92-7.86 (m, 2H), 7.64-7.56 (m, 2H), 7.50-7.43 (m, 2H), 7.24 (dd, J=8.0, 4.8 Hz, 1H), 4.20 (s, 2H), 3.74 (s, 3H), 2.94-2.83 (m, 1H), 1.03-0.95 (m, 2H), 0.86-0.74 (m, 2H).

### Example 25. Synthesis of N-methyl-3-[7-[[4-[1-methyl-4-(trifluoromethyl)imidazol-2-yl]phenyl]methyl]-5H-pyrrolo[3,2-d]pyrimidin-2-yl]pyridin-2-amine (Compound 58)

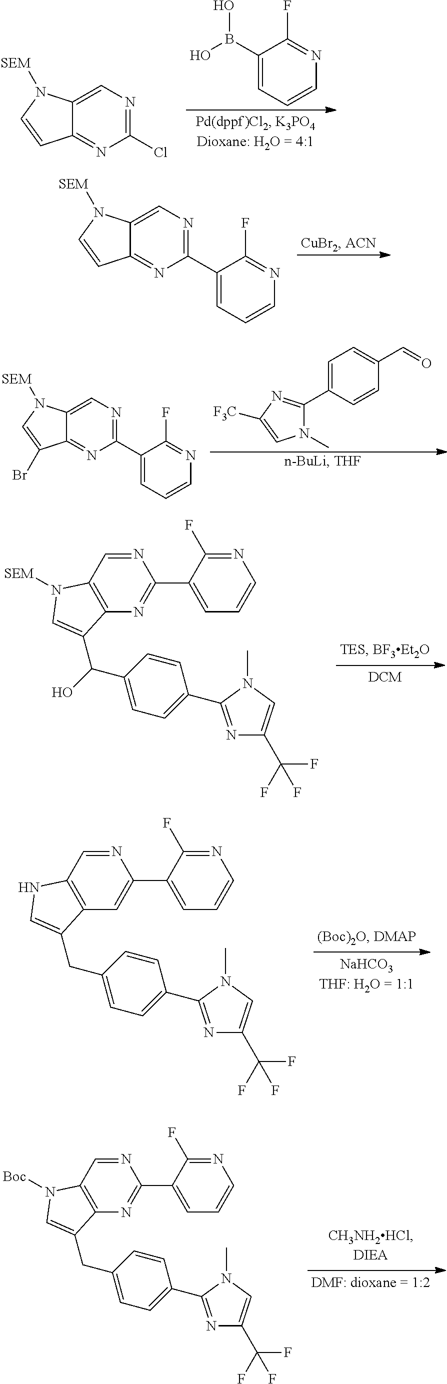

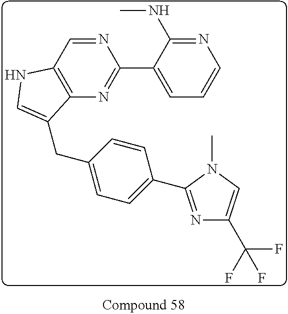 [SMILES: CNc1ncccc1-c1ncc2[nH]cc(Cc3ccc(-c4nc(C(F)(F)F)cn4C)cc3)c2n1]

### 2-[[2-(2-fluoro-3-pyridyl)pyrrolo[3,2-d]pyrimidin-5-yl]methoxy]ethyl-trimethyl-silane

To a solution of 2-chloro-5-[[2-(trimethylsilyl)ethoxy]methyl]pyrrolo[3,2-d]pyrimidine (1.00 g, 3.52 mmol) and 2-fluoropyridin-3-ylboronic acid (0.745 g, 5.28 mmol) in dioxane (10 mL) and H₂O (2.5 mL) were added potassium phosphate tribasic (2.24 g, 10.57 mmol) and 1,1′-Bis(diphenylphosphino)ferrocene-palladium(II)dichloride dichloromethane complex (288 mg, 0.35 mmol). The solution was stirred for 16 h at 100° C., then diluted with EtOAc (50 mL). The organic layer were washed with brine (3×10 mL), dried over anhydrous Na₂SO₄. After filtration, the filtrate was concentrated under reduced pressure. The residue was purified by silica gel column chromatography, eluted with PE:EtOAc=2:3 to afford 2-fluoro-3-(5-[[2-(trimethylsilyl)ethoxy]methyl]pyrrolo[3,2-d]pyrimidin-2-yl)pyridine (860 mg, 2.5 mmol, 71%) as a yellow oil. MS: m/z=345.10 [M+H]⁺. ¹H NMR (400 MHz, Chloroform-d) δ 9.13 (s, 1H), 8.59-8.55 (m, 1H), 8.35-8.30 (m, 1H), 7.64 (d, J=3.2 Hz, 1H), 7.38-7.34 (m, 1H), 6.84 (s, 1H), 5.61 (s, 2H), 3.58-3.49 (m, 2H), 1.00-0.89 (m, 2H), 0.00 (s, 9H).

### 2-[[7-bromo-2-(2-fluoro-3-pyridyl)pyrrolo[3,2-d]pyrimidin-5-yl]methoxy]ethyl-trimethyl-silane

To a stirred solution of 2-fluoro-3-(5-[[2-(trimethylsilyl)ethoxy]methyl]pyrrolo[3,2-d]pyrimidin-2-yl)pyridine (810.00 mg, 2.35 mmol) in MeCN (16 mL) was added cupric bromide (1.57 g, 7.05 mmol) at room temperature. The resulting mixture was stirred for 16 h at room temperature then 7 M NH₃ in MeOH (10 mL) was added at room temperature. After the resulting mixture was stirred for additional 30 min at room temperature, it was diluted with EtOAc (200 mL). The combined organic layers were washed with brine (3×50 mL), dried over anhydrous Na₂SO₄. After filtration, the filtrate was concentrated under reduced pressure. The residue was purified by silica gel column chromatography, eluted with PE:EtOAc=2:1 to afford 3-(7-bromo-5-[[2-(trimethylsilyl)ethoxy]methyl]pyrrolo[3,2-d]pyrimidin-2-yl)-2-fluoropyridine (940 mg, 2.21 mmol, 94%) as a yellow solid. MS: m/z=426.05, 428.05 [M+H]⁺. ¹H NMR (400 MHz, DMSO-d₆) δ 9.14 (s, 1H), 8.66-8.62 (m, 1H), 8.34-8.32 (m, 1H), 7.69 (s, 1H), 7.39-7.35 (m, 1H), 5.59 (s, 2H), 3.57-3.53 (s, 2H), 0.97-0.93 (m, 2H), 0.00 (s, 9H).

### [2-(2-fluoro-3-pyridyl)-5-(2-trimethylsilylethoxymethyl)pyrrolo[3,2-d]pyrimidin-7-yl]-[4-[l-methyl-4-(trifluoromethyl)imidazol-2-yl]phenyl]methanol

To a solution of 3-(7-bromo-5-[[2-(trimethylsilyl)ethoxy]methyl]pyrrolo[3,2-d]pyrimidin-2-yl)-2-fluoropyridine (0.90 g, 2.13 mmol) in THE (10 mL), n-butyllithium solution (2.5 M in hexane, 1.02 mL, 2.55 mmol) was added dropwise at −70° C. under N₂ atmosphere. The reaction mixture was stirred at −70° C. for 10 mins. Then a solution of 4-[1-methyl-4-(trifluoromethyl)imidazol-2-yl]benzaldehyde (540 mg, 2.13 mmol) in 5 mL THE was added dropwise at −70° C., and the mixture was stirred for another 1 h. The reaction was quenched with saturated aqueous ammonium chloride (30 mL), and then the mixture was extracted with EtOAc (2×15 mL). The combined organic extracts were washed with brine (10 mL), dried over anhydrous Na₂SO₄, After filtration, the filtrate was concentrated under vacuum. The residue was purified by silica gel column chromatography, eluted with PE:EtOAc=1:4 to afford [2-(2-fluoropyridin-3-yl)-5-[[2-(trimethylsilyl)ethoxy]methyl]pyrrolo[3,2-d]pyrimidin-7-yl]([4-[1-methyl-4-(trifluoromethyl)imidazol-2-yl]phenyl])methanol (260 mg, 0.435 mmol, 20%) as a yellow solid. MS: m/z=599.10 [M+H]⁺; ¹H NMR (400 MHz, Chloroform-d) δ crude

### 2-fluoro-3-(S-[[2-(trimethylsilyl)ethoxy]methyl]pyrrolo[3,2-d]pyrimidin-2-yl)pyridine

To a stirred solution of [2-(2-fluoropyridin-3-yl)-5-[[2-(trimethylsilyl)ethoxy]methyl]pyrrolo[3,2-d]pyrimidin-7-yl]([4-[1-methyl-4-(trifluoromethyl)imidazol-2-yl]phenyl])methanol (250 mg, 0.41 mmol) in DCM (3 mL) were added triethylsilane (971 mg, 8.4 mmol) and boron trifluoride etherate (1.19 g, 8.36 mmol) dropwise at 0° C. The resulting mixture was stirred at 0° C. for 1 h. The temperature was warmed to room temperature naturally. The resulted mixture was stirred for additional 16 h at room temperature. The reaction solution was quenched by saturated aq. NaHCO₃ (100 mL). The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE:EtOAc=2:1 to afford 2-fluoro-3-[7-([4-[1-methyl-4-(trifluoromethyl)imidazol-2-yl]phenyl]methyl)-5H-pyrrolo[3,2-d]pyrimidin-2-yl]pyridine (120 mg, 0.265 mmol, 64%) as a white solid. MS: m/z=453.05 [M+H]⁺.

### tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate

To a stirring mixture of 2-fluoro-3-[7-([4-[1-methyl-4-(trifluoromethyl)imidazol-2-yl]phenyl]methyl)-5H-pyrrolo[3,2-d]pyrimidin-2-yl]pyridine (57 mg, 0.12 mmol) and sodium bicarbonate (42 mg, 0.5 mmol) in THF (1 mL) and H₂O (1 mL) were added di-tert-butyl dicarbonate (110 mg, 0.5 mmol) and DMAP (1.5 mg, 0.01 mmol) at room temperature. After the resulting mixture was stirred for 16 h at room temperature, it was diluted by water (100 mL) and extracted with EtOAc (3×20 mL). The combined organic layers were washed with brine (3×20 mL), dried over anhydrous Na₂SO₄. After filtration, the filtrate was concentrated under reduced pressure. The residue was purified by Prep-TLC (PE:EtOAc=1:1) to afford tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate (50 mg, 0.091 mmol, 72%) as a white solid. ¹H NMR (400 MHz, Chloroform-d) δ 9.46 (s, 1H), 8.64-8.60 (m, 1H), 8.36-8.32 (m, 1H), 7.75 (s, 1H), 7.68-7.58 (m, 2H), 7.57-7.53 (m, 2H), 4.25 (s, 2H), 3.78 (s, 3H), 1.72 (s, 9H).

### N-methyl-3-[7-[[4-[1-methyl-4-(trifluoromethyl)imidazol-2-yl]phenyl]methyl]-5H-pyrrolo[3,2-d]pyrimidin-2-yl]pyridin-2-amine

To a stirred solution of tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate (40 mg, 0.07 mmol) in DMF (0.5 mL) and dioxane (1 mL) was added methylamine hydrochloride (34 mg, 0.5 mmol) and DIEA (44 mg, 0.64 mmol) at room temperature. The resulting mixture was irradiated with microwave for 2 h at 160° C. The residue was purified by reverse phase chromatography with the following conditions: Column: Spherical C18, 20-40 um, 40 g; Mobile Phase A: Water (10 mM aq. NH₄HCO₃), Mobile Phase B: ACN; Flow rate: 40 mL/min; Gradient (B %): 0% hold 5 min, 5%-40% within 10 min; 40% hold 5 min, 40%-60% within 20 min, 60% hold 3 min, 60%-95% within 2 min, 95% hold 5 min; Detector: UV 254 & 220 nm; RT: 30 min. The collected fractions were combined and concentrated under reduced pressure to afford N-methyl-3-[7-[[4-[1-methyl-4-(trifluoromethyl)imidazol-2-yl]phenyl]methyl]-5H-pyrrolo[3,2-d]pyrimidin-2-yl]pyridin-2-amine as a white solid. MS: m/z=464.10 [M+H]⁺. ¹H NMR (400 MHz, Methanol-d₄) δ 8.94 (s, 1H), 8.77 (dd, J=7.6, 2.0 Hz, 1H), 8.07 (dd, J=4.8, 2.0 Hz, 1H), 7.74 (s, 1H), 7.67 (s, 1H), 7.60 (d, J=8.0 Hz, 2H), 7.55 (d, J=8.4 Hz, 2H), 6.69 (dd, J=7.6, 2.0 Hz, 1H), 4.33 (s, 2H), 3.76 (s, 3H), 3.04 (s, 3H). ¹⁹F NMR (376 MHz, Methanol-d₄) δ −63.94

### Example 26. Synthesis of 7-[[4-[1-methyl-4-(trifluoromethyl)imidazol-2-yl]phenyl]methyl]-2-[2-(2,2,2-trifluoroethoxy)-3-pyridyl]-5H-pyrrolo[3,2-d]pyrimidine (Compound 79)

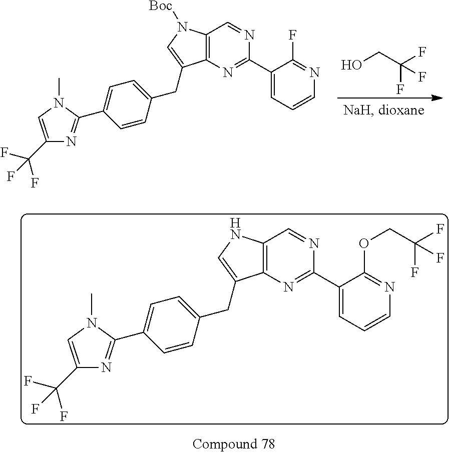 [SMILES: Cn1cc(C(F)(F)F)nc1-c1ccc(Cc2cn(C(=O)OC(C)(C)C)c3cnc(-c4cccnc4F)nc23)cc1 ; OCC(F)(F)F ; Cn1cc(C(F)(F)F)nc1-c1ccc(Cc2c[nH]c3cnc(-c4cccnc4OCC(F)(F)F)nc23)cc1]

### 7-[[4-[1-methyl-4-(trifluoromethyl)imidazol-2-yl]phenyl]methyl]-2-[2-(2,2,2-trifluoroethoxy)-3-pyridyl]-5H-pyrrolo[3,2-d]pyrimidine

A solution of NaH (2.9 mg, 0.07 mmol, 60% in oil) and trifluoroethanol (7 mg, 0.07 mmol) in dioxane (0.5 mL) was stirred for 15 min, at room temperature. To above mixture tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate (prepared as above) (20 mg, 0.036 mmol) dissolved in dioxane (1.0 mL) was added dropwise at room temperature. The resulting mixture was stirred for additional 16 h at 100° C. After concentrated under reduced pressure, the residue was purified by Prep-TLC (Dichloromethane:Methanol=15:1) to afford crude product (12 mg) as a light yellow oil. The crude product was purified by reverse phase chromatography with the following conditions: Column: Spherical C18, 20-40 um, 40 g; Mobile Phase A: 10 mM aq. NH₄HCO₃. Mobile Phase B: ACN; Flow rate: 40 mL/min; Gradient (B %): 0% hold 5 min, 5%-50% within 10 min; 50%-70% within 20 min, 70%-95% within 5 min, 95% hold 5 min; Detector: UV 254 & 220 nm; RT: 25 min. The collected fractions were combined, concentrated and then lyophilized to afford 3-[7-([4-[1-methyl-4-(trifluoromethyl)imidazol-2-yl]phenyl]methyl)-5H-pyrrolo[3,2-d]pyrimidin-2-yl]-2-(2,2,2-trifluoroethoxy)pyridine (5.9 mg, 11.1 μmol, 30% yield) as a light yellow solid. MS: m/z=533.10 [M+H]⁺. ¹H NMR (400 MHz, Methanol-d₄) δ 9.30 (s, 1H), 8.55-8.46 (m, 3H), 7.85 (s, 1H), 7.68-7.58 (m, 4H), 7.42 (dd, J=7.6, 5.2 Hz, 1H), 5.21-5.10 (m, 2H), 4.43 (s, 2H), 3.81 (s, 3H). ¹⁹F NMR (400 MHz, Methanol-d₄) δ −63.69, −75.02

### Example 27. Synthesis of 2-isopropoxy-3-[7-([4-[1-methyl-4-(trifluoromethyl)imidazol-2-yl]phenyl]methyl)-5H-pyrrolo[3,2-d]pyrimidin-2-yl]pyridine (Compound 55)

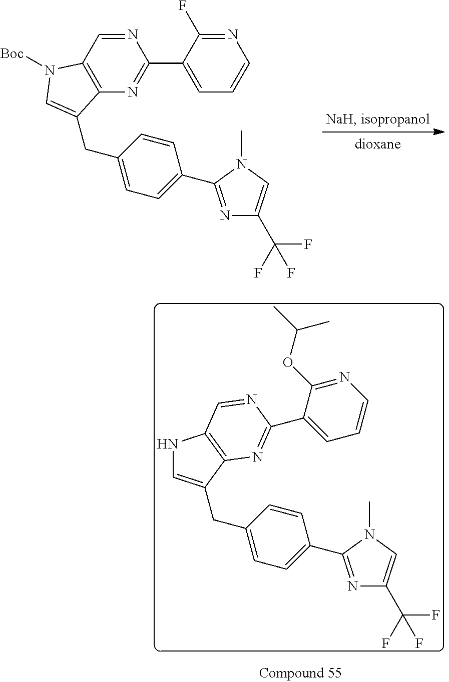

### 2-isopropoxy-3-[7-([4-[1-methyl-4-(trifluoromethyl)imidazol-2-yl]phenyl]methyl)-5H-pyrrolo[3,2-d]pyrimidin-2-yl]pyridine

To a stirring mixture of isopropyl alcohol (16 mg, 0.27 mmol) in dioxane (1.0 mL) was added sodium hydride (11 mg, 0.27 mmol, 60%). The mixture was stirred for 15 mins at room temperature. To the above mixture was added tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate (see preparation above) (30 mg, 0.054 mmol). The resulting mixture was stirred for additional 16 h at 100° C. The residue was purified directly by RP-Flash with the following conditions: Column: Spherical C18, 20-40 um, 40 g; Mobile Phase A: Water (10 mM aq. NH₄HCO₃), Mobile Phase B: MeCN; Flow rate: 40 mL/min; Gradient (B %): 0% hold 5 min, 5%-40% within 10 min; 40%-60% within 20 min, 60%-95% within 5 min, 95% hold 5 min; Detector: UV 254 & 220 nm; RT: 25 min. The collected fractions were combined and concentrated under reduced pressure to afford 2-isopropoxy-3-[7-([4-[1-methyl-4-(trifluoromethyl)imidazol-2-yl]phenyl]methyl)-5H-pyrrolo[3,2-d]pyrimidin-2-yl]pyridine (1.7 mg, 6%) as an off-white solid. MS: m/z=493.15 [M+H]⁺. ¹H NMR (400 MHz, Methanol-d₄) δ 8.92 (s, 1H), 8.28-8.21 (m, 1H), 7.93-7.86 (m, 1H), 7.73-7.65 (m, 2H), 7.58-7.53 (m, 4H), 7.11-7.04 (m, 1H), 5.42-5.31 (m, 1H), 4.31 (s, 2H), 3.77 (s, 3H), 1.28 (d, J=6.4 Hz, 6H). ¹⁹F NMR (400 MHz, Methanol-d₄) δ −63.92

### Example 28. Synthesis of 2-cyclopropoxy-3-[7-([4-[1-methyl-4-(trifluoromethyl)imidazol-2-yl]phenyl]methyl)-5H-pyrrolo[3,2-d]pyrimidin-2-yl]pyridine (Compound 41) & 3-[7-([4-[1-methyl-4-(trifluoromethyl)imidazol-2-yl]phenyl]methyl)-5H-pyrrolo[3,2-d]pyrimidin-2-yl]pyridin-2-ol (Compound 56)

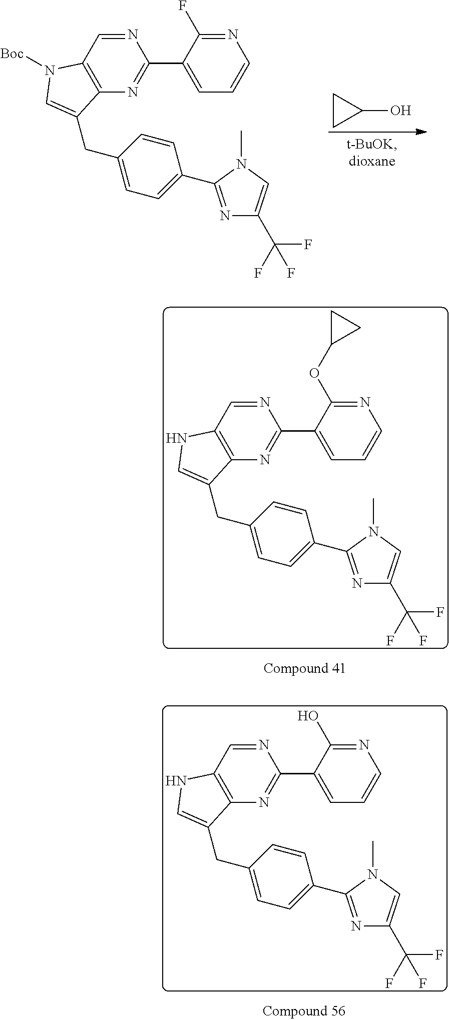

### 2-cyclopropoxy-3-[7-([4-[1-methyl-4-(trifluoromethyl)imidazol-2-yl]phenyl]methyl)-5H-pyrrolo[3,2-d]pyrimidin-2-yl]pyridine and 3-[7-([4-[1-methyl-4-(trifluoromethyl)imidazol-2-yl]phenyl]methyl)-5H-pyrrolo[3,2-d]pyrimidin-2-yl]pyridin-2-ol

To a stirred solution of tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate (prepared as above) (60 mg, 0.10 mmol) and cyclopropanol (63 mg, 1.1 mmol) in dioxane (1.0 mL) were added potassium tert-butoxide (1 M in THF, 1.08 mL, 1.08 mmol) at room temperature. The resulting mixture was stirred for 16 h at 100° C. then was purified directly by reverse phase chromatography with the following conditions: Column: Spherical C18, 20-40 um, 40 g; Mobile Phase A: Water (10 mM aq. NH₄HCO₃), Mobile Phase B: MeCN; Flow rate: 40 mL/min; Gradient (B %): 0% hold 5 min, 5%-50% within 10 min; 50% hold 2 min, 50%-70% within 20 min, 70%-95% within 5 min, 95% hold 5 min; Detector: UV 254 & 220 nm; RT1=16 min, RT2: 25 min. The collected fractions (the first peak, RT1=16 min) were collected, concentrated under reduced pressure and then lyophilized overnight to afford 3-[7-([4-[1-methyl-4-(trifluoromethyl)imidazol-2-yl]phenyl]methyl)-5H-pyrrolo[3,2-d]pyrimidin-2-yl]pyridin-2-ol (7.5 mg, 16.7 umol, 16% yield) as a white solid. MS: m/z=451.15 [M+H]⁺. ¹H NMR (400 MHz, Methanol-d₄) δ 9.20-9.17 (m, 2H), 8.37 (s, 1H), 7.92 (d, J=4.8 Hz, 1H), 7.70 (s, 1H), 7.63-7.58 (m, 4H), 6.87-6.83 (m, 1H), 4.41 (s, 2H), 3.77 (s, 3H). ¹⁹F NMR (400 MHz, Methanol-d₄) −63.95

Another collected fractions (the first peak, RT2=25 min) were collected, concentrated under reduced pressure and then lyophilized overnight to afford a crude product (3 mg). The crude product was further purified by Prep-HPLC with the following conditions: Column: XBridge Shield RP18 OBD Column, 19×250 mm, 10 um; Mobile Phase A: Water (10 mM aq. NH₄HCO₃), Mobile Phase B: MeCN; Flow rate: 60 mL/min; Gradient: 30% B to 44% B in 10 min; Detector: UV 254 & 210 nm; RT: 9.48 min. The collected fractions were combined, concentrated under reduced pressure and then lyophilized overnight to give 2-cyclopropoxy-3-[7-([4-[1-methyl-4-(trifluoromethyl)imidazol-2-yl]phenyl]methyl)-5H-pyrrolo[3,2-d]pyrimidin-2-yl]pyridine (1.2 mg, 2.45 umol, 2% yield) as an off-white solid. MS: m/z=491.20 [M+H]⁺. ¹H NMR (400 MHz, Methanol-d₄) δ 8.91 (s, 1H), 8.33-8.27 (m, 1H), 8.01-7.93 (m, 1H), 7.75-7.66 (m, 2H), 7.62-7.50 (m, 4H), 7.21-7.14 (m, 1H), 4.34-4.29 (m, 3H), 3.78 (s, 3H), 0.76-0.72 (m, 2H), 0.68-0.64 (m, 2H). ¹⁹F NMR (400 MHz, Methanol-d₄) −63.92

### Example 29. Synthesis of 7-[[4-[1-methyl-4-(trifluoromethyl)imidazol-2-yl]phenyl]methyl]-2-(2-propoxy-3-pyridyl)-5H-pyrrolo[3,2-d]pyrimidine (Compound 34)

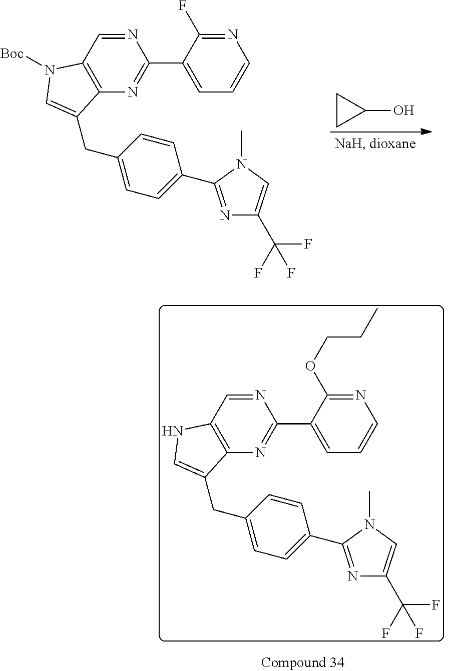

### 7-[[4-[1-methyl-4-(trifluoromethyl)imidazol-2-yl]phenyl]methyl]-2-(2-propoxy-3-pyridyl)-5H-pyrrolo[3,2-d]pyrimidine

To a solution of cyclopropanol (2.9 mg, 0.05 mmol) in 1,4-dioxane (0.5 mL) was added sodium hydride (3.6 mg, 0.09 mmol, 60%) at 0° C. The reaction was stirred for 15 min at mom temperature. To the above mixture was added tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate (25.00 mg, 0.04 mmol) dissolved in dioxane (0.5 mL) at room temperature. The resulting mixture was stirred for additional 16 h at 100° C. After evaporation, the residue was purified directly by RP-Flash with the following conditions: Column: Spherical C18, 20-40 μm, 40 g; Mobile Phase A: 10 mM aq. NH₄HCO₃, Mobile Phase B: MeCN; Flow rate: 40 mL/min; Gradient (B %): 0% hold 5 min, 0%-50% within 15 min; 50%-95% within 2 min, 95% hold 5 min; Detector: UV 254 & 220 nm; RT: 20 min. The collected fractions were combined and concentrated under reduced pressure to afford 7-[[4-[1-methyl-4-(trifluoromethyl)imidazol-2-yl]phenyl]methyl]-2-(2-propoxy-3-pyridyl)-5H-pyrrolo[3,2-d]pyrimidine (0.9 mg, 1.83 μmol, 4% yield). MS: m/z=493.20 [M+H]⁺. ¹H NMR (400 MHz, Methanol-d₄) δ 8.93 (s, 1H), 8.28-8.21 (m, 1H), 7.99-7.92 (m, 1H), 7.69 (d, J=8.0 Hz, 2H), 7.61-7.49 (m, 4H), 7.15-7.07 (m, 1H), 4.35-4.28 (m, 4H), 3.77 (s, 3H), 1.77-1.63 (m, 2H), 0.92 (t, J=7.6 Hz, 3H). ¹⁹F NMR (400 MHz, Methanol-d₄) δ −63.93.

### Example 30. Synthesis of 2-(2-methoxy-3-pyridyl)-7-[[4-[1-methyl-4-(trifluoromethyl)imidazol-2-yl]phenyl]methyl]-5H-pyrrolo[3,2-d]pyrimidine (Compound 11)

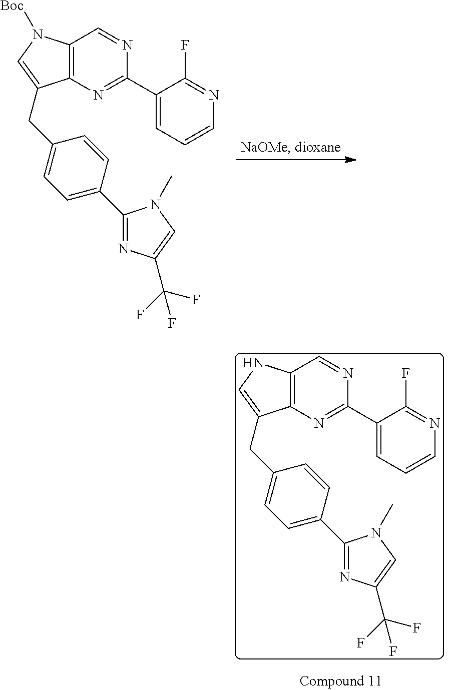 [SMILES: Cn1cc(C(F)(F)F)nc1-c1ccc(Cc2cn(C(=O)OC(C)(C)C)c3cnc(-c4cccnc4F)nc23)cc1 ; Cn1cc(C(F)(F)F)nc1-c1ccc(Cc2c[nH]c3cnc(-c4cccnc4F)nc23)cc1]

### 2-(2-methoxy-3-pyridyl)-7-[[4-[1-methyl-4-(trifluoromethyl)imidazol-2-yl]phenyl]methyl]-5H-pyrrolo[3,2-d]pyrimidine

To a stirring solution of tert-butyl 2-(2-fluoropyridin-3-yl)-7-([4-[1-methyl-4-(trifluoromethyl)imidazol-2-yl]phenyl]methyl)pyrrolo[3,2-d]pyrimidine-5-carboxylate (20 mg, 0.036 mmol) in 1,4-dioxane (0.50 mL) was added sodium methanolate (12 mg, 0.2 mmol) at 25° C. The resulting mixture was stirred for 16 h at 100° C. After evaporation, the residue was purified directly by Prep-HPLC with the following conditions: Column: XBridge Shield RP18 OBD Column, 30×150 mm, 5 μm; Mobile Phase A: Water (10 mM aq. NH₄HCO₃+0.1% NH₃·H₂O), Mobile Phase B: MeCN; Flow rate: 60 mL/min; Gradient: 35% B to 70% B in 9 min; Detector: UV 254 & 210 nm; RT: 7.23 min). The collected fractions were combined and concentrated under reduced pressure to afford 2-(2-methoxy-3-pyridyl)-7-[[4-[1-methyl-4-(trifluoromethyl)imidazol-2-yl]phenyl]methyl]-5H-pyrrolo[3,2-d]pyrimidine (3.2 mg, 6.89 μmol, 19% yield) as a light yellow solid. MS: m/z=465.15 [M+H]⁺. ¹H NMR (400 MHz, Methanol-d₄) δ 8.98 (s, 1H), 8.30-8.23 (m, 1H), 8.05-7.98 (m, 1H), 7.90-7.84 (m, 2H), 7.65-7.53 (m, 4H), 7.17-7.10 (m, 1H), 4.31 (s, 2H), 4.01 (s, 3H), 3.77 (s, 3H). ¹⁹F NMR (400 MHz, Methanol-d₄) δ −63.94.

### Example 31. Synthesis of [2-[2-(difluoromethoxy)-3-pyridyl]-5H-pyrrolo[3,2-d]pyrimidin-7-yl]-[4-[1-(1-methylazetidin-3-yl)-4-(trifluoromethyl)imidazol-2-yl]phenyl]methanol (Compound 38) and 2-[2-(difluoromethoxy)-3-pyridyl]-7-[[4-[1-(1-methylazetidin-3-yl)-4-(trifluoromethyl)imidazol-2-yl]phenyl]methyl]-5H-pyrrolo[3,2-d]pyrimidine (Compound 20)

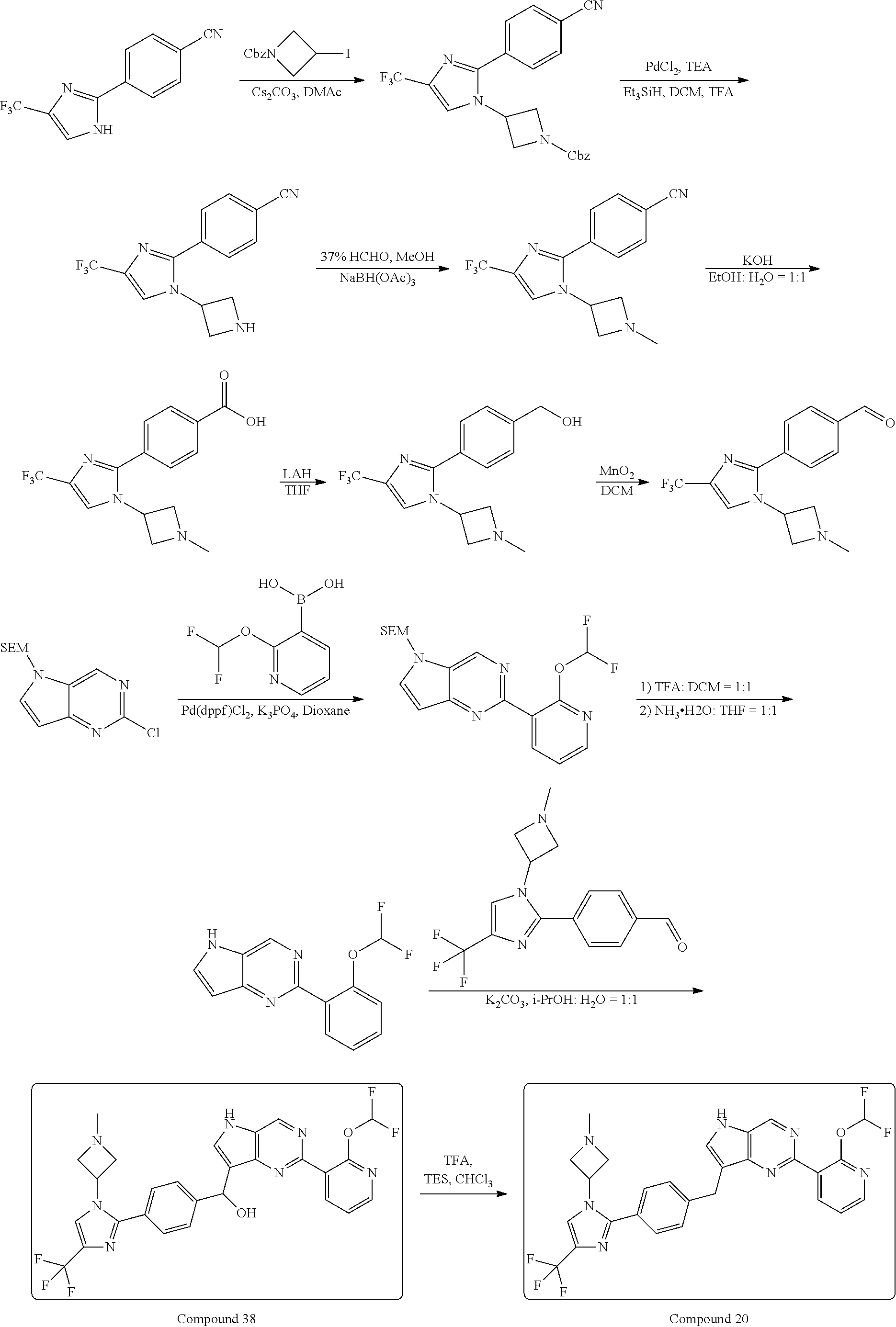

### benzyl 3-[2-(4-cyanophenyl)-4-(trifluoromethyl)imidazol-1-yl]azetidine-1-carboxylate

To a solution of 4-[4-(trifluoromethyl)-1H-imidazol-2-yl]benzonitrile (700 mg, 2.95 mmol) in N,N-dimethylacetamide (20 mL) was added benzyl 3-iodoazetidine-1-carboxylate (1.12 g, 3.54 mmol) and cesium carbonate (1.92 g, 5.90 mmol) at 25 TC. The resulting mixture was stirred at 130° C. for 16 h then diluted with EA (100 mL). The organic phase was washed by saturated aqueous sodium chloride (30 mL×3). The organic layer was dried over anhydrous sodium sulfate. After filtration, the filtrate was concentrated under reduced pressure. The residue was purified by silica gel column chromatography, eluted with 25%-45% EtOAc in PE to give 321 mg crude product. The crude product was purified by RP-Flash chromatography with the following conditions: Column: C18 spherical, 20˜35 um, 100A, 330 g; Mobile Phase A: Water, Mobile Phase B: MeCN; Flow rate: 100 mL/min; Gradient: 5% B to 5% B in 5 min, 5% B to 50% B in 5 min, 50% B to 80% B in 30 min, 80% B to 95% B in 5 min. Detector: UW 254 & 220 nm; RT: 27 min to afford benzyl 3-[2-(4-cyanophenyl)-4-(trifluoromethyl)imidazol-1-yl]azetidine-1-carboxylate (202 mg, 473.74 μmol, 16% yield) as a yellow solid. MS: m/z=427.10 [M+H]⁺ ¹H NMR (400 MHz, Chloroform-d) δ 7.85-7.76 (m, 3H), 7.66-7.59 (m, 2H), 7.45-7.33 (m, 5H), 5.16 (s, 2H), 5.15-5.08 (m, 1H), 4.58-4.54 (m, 2H), 4.25-4.21 (m, 2H).

### 4-[1-(azetidin-3-yl)-4-(trifluoromethyl)imidazol-2-yl]benzonitrile

To a solution of benzyl 3-[2-(4-cyanophenyl)-4-(trifluoromethyl)imidazol-1-yl]azetidine-1-carboxylate (1.86 g, crude, 4.36 mmol) in dichloromethane (44 mL) was added palladium chloride (77 mg, 436 μmol) and triethylamine (441 mg, 4.3 mmol). Triethylsilane (1.52 g, 13.09 mmol) was added (2×760 mg, over 2 h) at 25° C. The reaction mixture was stirred at 25° C. for 1 h and then trifluoroacetic acid (5.6 mL) was added. After 0.5 h the reaction was basified by 2 M sodium hydroxide, extracted by DCM (50 mL×3), dried sodium sulfate. After filtration, the filtrate was concentrated under reduced pressure. The residue was purified by silica gel column chromatography, eluted with 10%-25% MeOH (containing 5% NH₃·H₂O) in DCM to give 4-[1-(azetidin-3-yl)-4-(trifluoromethyl)imidazol-2-yl]benzonitrile (225 mg, 769.87 μmol, 31% yield) as a brown solid. MS: m/z=293.05 [M+H]⁺. ¹H NMR (400 MHz, Chloroform-d) δ 7.92 (s, 1H), 7.82-7.79 (m, 2H), 7.70-7.63 (m, 2H), 5.19-5.12 (m, 1H), 4.17-4.08 (m, 2H), 3.93-3.85 (m, 2H).

### 4-[1-(1-methylazetidin-3-yl)-4-(trifluoromethyl)imidazol-2-yl]benzonitrile

To a solution of 4-[1-(azetidin-3-yl)-4-(trifluoromethyl)imidazol-2-yl]benzonitrile (785 mg, 2.69 mmol) in methanol (12 mL) was added formaldehyde solution (37% in water, 6 mL), then was stirred at 25° C. for 1 h. The resulting solution was added sodium triacetoxyborohydride (2.85 g, 13.43 mmol) at 25° C., and then stirred at 25° C. for 2 h. The resulted solution was diluted with DCM (100 mL) and adjusted pH to 7˜8 using saturated aqueous sodium bicarbonate. The organic layer was separated, dried over anhydrous sodium sulfate. After filtration, the filtrate was concentrated under reduced pressure. The residue was purified by silica gel column chromatography, eluted with 0%-15% MeOH in DCM to give 4-[1-(1-methylazetidin-3-yl)-4-(trifluoromethyl)imidazol-2-yl]benzonitrile (810 mg, 2.64 mmol, 98% yield) as a yellow solid. MS: m/z=307.10 [M+H]⁺. ¹H NMR (300 MHz, Chloroform-d) δ 7.80 (d, J=8.0 Hz, 2H), 7.65 (d, J=8.0 Hz, 2H), 5.01-4.89 (m, 1H), 4.09-3.76 (m, 4H), 2.53 (s, 3H).

### 4-[1-(1-benzyloxycarbonylazetidin-3-yl)-4-(trifluoromethyl)imidazol-2-yl]benzoic Acid

To a solution of benzyl 3-[2-(4-cyanophenyl)-4-(trifluoromethyl)imidazol-1-yl]azetidine-1-carboxylate (670 mg, 1.57 mmol) in water (5 mL) and ethanol (5 mL) were added potassium hydroxide (441 mg, 7.9 mmol) at 15° C., then was stirred at 100° C. for 1 h. The resulted solution was adjusted pH to 5-6 using 1 M aq. HCl. The resulted mixture was purified by RP-Flash chromatography with the following conditions: Column: C18 spherical, 20-35 um, 100A, 80 g; Mobile Phase A: 0.05 mM aq. HCl, Mobile Phase B: MeCN; Flow rate: 60 mL/min; Gradient: 5% B to 5% B in 5 min, 5% B to 30% B in 25 min, 30% B to 95% B in 5 min, Detector: UV 254 & 220 nm; RT: 15 min to give 4-[1-(1-benzyloxycarbonylazetidin-3-yl)-4-(trifluoromethyl)imidazol-2-yl]benzoic acid (650 mg, 1.46 mmol, 92% yield) as a white solid. MS: m/z=326.00 [M+H]⁺. ¹H NMR (400 MHz, Methanol-d₄) δ 8.48 (s, 1H), 8.25-8.23 (m, 2H), 7.75-7.71 (m, 2H), 5.75-5.35 (m, 1H), 4.86-4.67 (m, 2H), 4.55-4.48 (m, 2H), 3.08 (s, 3H).

### [4-[1-(1-methylazetidin-3-yl)-4-(trifluoromethyl)imidazol-2-yl]phenyl]methanol

To a solution of 4-[1-(1-methylazetidin-3-yl)-4-(trifluoromethyl)imidazol-2-yl]benzoic acid (650 mg, 2.0 mmol) in THF (14 mL) was added lithium aluminum hydride (152 mg, 4.0 mmol) at 0° C., then was stirred at 25° C. for 16 h. The resulted solution was quenched by sodium sulfate decahydrate. The resulting mixture was stirred at 15° C. for 0.5 h, filtrated. The filtrate was concentrated under reduced pressure. The residue was purified by silica gel column chromatography, eluted with 5%-20% MeOH in DCM to afford [4-[1-(1-methylazetidin-3-yl)-4-(trifluoromethyl)imidazol-2-yl]phenyl]methanol (480 mg, 1.54 mmol, 77% yield) as a yellow solid. MS: m/z=312.10 [M+H]⁺.

### 4-[1-(1-methylazetidin-3-yl)-4-(trifluoromethyl)imidazol-2-yl]benzaldehyde

To a solution of [4-[1-(1-methylazetidin-3-yl)-4-(trifluoromethyl)imidazol-2-yl]phenyl]methanol (300 mg, 964 μmol) in dichloromethane (6 mL) was added manganese dioxide (838 mg, 9.6 mmol) at 15° C., then was stirred at 40° C. for 16 h. The resulted solution was filtrated. The filtrate was concentrated under reduced pressure. The residue was purified by silica gel column chromatography, eluted with 20%-45% EtOAc in PE to afford 4-[1-(1-methylazetidin-3-yl)-4-(trifluoromethyl)imidazol-2-yl]benzaldehyde (274 mg, 885.9 μmol, 91% yield) as an orange oil. MS: m/z=310.10 [M+H]⁺. ¹H NMR (400 MHz, Chloroform-d) δ 10.07 (s, 1H), 8.02-7.95 (m, 2H), 7.93 (s, 1H), 7.72-7.65 (m, 2H), 4.93-4.87 (m, 1H), 3.77-3.69 (m, 2H), 3.48-3.37 (m, 2H), 2.42 (s, 3H).

### 2-[[2-[2-(difluoromethoxy)-3-pyridyl]pyrrolo[3,2-d]pyrimidin-5-yl]methoxy]ethyl-trimethyl-silane

To a solution of [2-(difluoromethoxy)-3-pyridyl]boronic acid (0.5 g, 2.65 mmol) in 1,4-dioxane (10 mL) were added [1,1′-bis(diphenylphosphino)ferrocene]dichloropalladium(yl) (193.65 mg, 264.66 μmol), potassium phosphate (1.12 g, 5.29 mmol) and 2-[(2-chloropyrrolo[3,2-d]pyrimidin-5-yl)methoxy]ethyl-trimethyl-silane (751.17 mg, 2.65 mmol) at 25° C. under argon atmosphere. The resulting mixture was stirred at 100° C. for 16 h then diluted by DCM (100 mL). The resulting mixture was filtrated. The filtrate was concentrated under reduced pressure. The residue was purified by silica gel column chromatography, eluted with 35%-50% EtOAc in PE to afford 2-[[2-[2-(difluoromethoxy)-3-pyridyl]pyrrolo[3,2-d]pyrimidin-5-yl]methoxy]ethyl-trimethyl-silane (703 mg, 1.79 mmol, 67% yield) as a light-white solid. MS: m/z=393.05 [M+H]⁺. ¹H NMR (400 MHz, Chloroform-d) δ 9.12 (s, 1H), 8.34 -8.20 (m, 2H), 7.84-7.39 (m, 2H), 7.28-7.25 (m, 1H), 6.81 (s, 1H), 5.60 (s, 2H), 3.59-3.51 (m, 2H), 1.00-0.86 (m, 2H), −0.01 (s, 9H).

### 2-[2-(difluoromethoxy)phenyl]-5H-pyrrolo[3,2-d]pyrimidine

To a solution of trifluoroacetic acid (7.5 mL) in dichloromethane (7.5 mL) was added 2-[[2-[2-(difluoromethoxy)-3-pyridyl]pyrrolo[3,2-d]pyrimidin-5-yl]methoxy]ethyl-trimethyl-silane (683 mg, 1.74 mmol) at 25° C., then was stirred at 25° C. for 2 h. The resulted solution was concentrated under reduced pressure. The residue was added tetrahydrofuran (2.5 mL) and ammonia solution (25%, 2.5 mL), then was stirred at 25° C. for 10 min. The resulted solution was concentrated under reduced pressure. The residue was purified RP-Flash chromatography with the following conditions: Column: C18 spherical, 20˜35 um, 100A, 80 g; Mobile Phase A: 10 mM aq. NH₄HCO₃, Mobile Phase B: MeCN; Flow rate: 60 mL/min; Gradient: 5% B to 5% B in 5 min, 5% B to 20% B in 5 min, 20% B to 45% B in 25 min, 45% B to 95% B in 5 min; Detector: UV 254 & 220 nm; RT: 32 min to give 2-[2-(difluoromethoxy)phenyl]-5H-pyrrolo[3,2-d]pyrimidine (445 mg, 1.70 mmol, 97% yield) as a white solid. MS: m/z=263.15 [M+H]⁺. ¹H NMR (400 MHz, DMSO-d₆) δ 11.97 (s, 1H), 9.05 (s, 1H), 8.35-8.33 (m, 1H), 8.24 (d, J=8.0 Hz, 1H), 7.99 (s, 1H), 7.79 (t, J=73.2 Hz, 1H), 7.41 (m, 2H), 6.70 (d, J=4.0 Hz, 1H).

### [2-[2-(difluoromethoxy)-3-pyridyl]-5H-pyrrolo[3,2-d]pyrimidin-7-yl]-[4-[1-(1-methylazetidin-3-yl)-4-(trifluoromethyl)imidazol-2-yl]phenyl]methanol

To a solution of 2-[2-(difluoromethoxy)phenyl]-5H-pyrrolo[3,2-d]pyrimidine (231.42 mg, 885.91 umol) in isopropyl alcohol (6 mL) and water (6 mL) were added 4-[1-(1-methylazetidin-3-yl)4-(trifluoromethyl)imidazol-2-yl]benzaldehyde (274 mg, 885.91 umol) and potassium carbonate (146.93 mg, 1.06 mmol) at 15° C., then was stirred at 0° C. for 1 h. The resulting solution was stirred at 60° C. for 16 h then diluted by EA (50 mL). The organic phase was washed with saturated aqueous sodium chloride (20 mL×3). The organic layer was dried over anhydrous sodium sulfate. After filtration, the filtrate was concentrated under reduced pressure. The residue was purified by silica gel column, eluted with 8%-25% MeOH in DCM to give 207 mg crude product. The crude product was further purified by RP-Flash chromatography with the following conditions: Column: C18 spherical, 20-35 um, 100A, 80 g; Mobile Phase A: 10 mM aq. NH₄HCO₃, Mobile Phase B: MeCN; Flow rate: 60 mL/min; Gradient: 5% B to 5% B in 5 min, 5% B to 25% B in 5 min, 25% B to 40% B in 15 min, 40% B to 95% B in 5 min; Detector: UV 254 & 220 nm; RT: 17 min to [2-[2-(difluoromethoxy)-3-pyridyl]-5H-pyrrolo[3,2-d]pyrimidin-7-yl]-[4-[1-(1-methylazetidin-3-yl)-4-(trifluoromethyl)imidazol-2-yl]phenyl]methanol (120 mg, 209.97 μmol, 23% yield) as a white solid. MS: m/z=572.05 [M+H]⁺. ¹H NMR (400 MHz, Methanol-d₄) δ 8.97 (s, 1H), 8.35-8.33 (m, 1H), 8.22 (dd, J=7.2, 1.6 Hz, 1H), 8.14 (s, 1H), 7.80-7.77 (m, 3H), 7.67 (t, J=73.2 Hz, 1H), 7.52-7.48 (m, 2H), 7.39-7.36 (m, 1H), 6.45 (s, 1H), 4.97-4.90 (m, 1H), 3.74-3.70 (m, 2H), 3.45-3.41 (m, 2H), 2.40 (s, 3H).

### 2-[2-(difluoromethoxy)-3-pyridyl]-7-[[4-[J-(1-methylazetidin-3-yl)-4-(trifluoromethyl)imidazol-2-yl]phenyl]methyl]-5H-pyrrolo[3,2-d]pyrimidine

To a solution of [2-[2-(difluoromethoxy)-3-pyridyl]-5H-pyrrolo[3,2-d]pyrimidin-7-yl]-[4-[1-(1-methylazetidin-3-yl)-4-(trifluoromethyl)imidazol-2-yl]phenyl]methanol (82 mg, 143.48 umol) in chloroform (2 mL) was added 2,2,2-trifluoroacetic acid (1 mL) and triethylsilane (2 mL) at 25° C., then was stirred at 25° C. for 2 h. The resulted solution was concentrated under reduced pressure. The residue was co-evaporated with toluene (three times, 5 mL each) then purified by RP-Flash chromatography with the following conditions: Column: C18 spherical, 20-30 um, 100A, 40 g; Mobile Phase A: 10 mM aq. NH₄HCO₃, Mobile Phase B: MeCN; Flow rate: 40 mL/min; Gradient: 5% B to 5% B in 5 min, 5% B to 20% B in 5 min, 20% B to 45% B in 25 min, 45% B to 95% B in 5 min; Detector: UV 254 & 220 nm; RT: 24 min to give 2-[2-(difluoromethoxy)-3-pyridyl]-7-[[4-[1-(1-methylazetidin-3-yl)-4-(trifluoromethyl)imidazol-2-yl]phenyl]methyl]-5H-pyrrolo[3,2-d]pyrimidine (22.7 mg, 40.86 μmol, 28% yield) as a white solid. MS: m/z=556.10 [M+H]⁺. ¹H NMR (400 MHz, Methanol-d₄) δ 8.84 (s, 1H), 8.23-8.21 (m, 1H), 8.08 (dd, J=2.0, 7.6 Hz, 1H), 8.00 (s, 1H), 7.63 (s, 1H), 7.55 (t, J=73.2 Hz, 1H), 7.48-7.45 (m, 2H), 4.83-4.75 (m, 1H), 4.19 (s, 2H), 3.61-3.57 (m, 2H), 3.37-3.27 (m, 2H), 2.30 (m, 3H). ¹⁹F NMR (400 MHz, Methanol-d₄) δ −64.016, −89.810

### Example 32. Synthesis of 2-[2-(difluoromethoxy)-3-pyridyl]-7-[[4-[1-isopropyl-4-(trifluoromethyl)imidazol-2-yl]phenyl]methyl]-5H-pyrrolo[3,2-d]pyrimidine (Compound 28) and [2-[2-(difluoromethoxy)-3-pyridyl]-5H-pyrrolo[3,2-d]pyrimidin-7-yl]-[4-[1-isopropyl-4-(trifluoromethyl)imidazol-2-yl]phenyl]methanol (Compound 52)

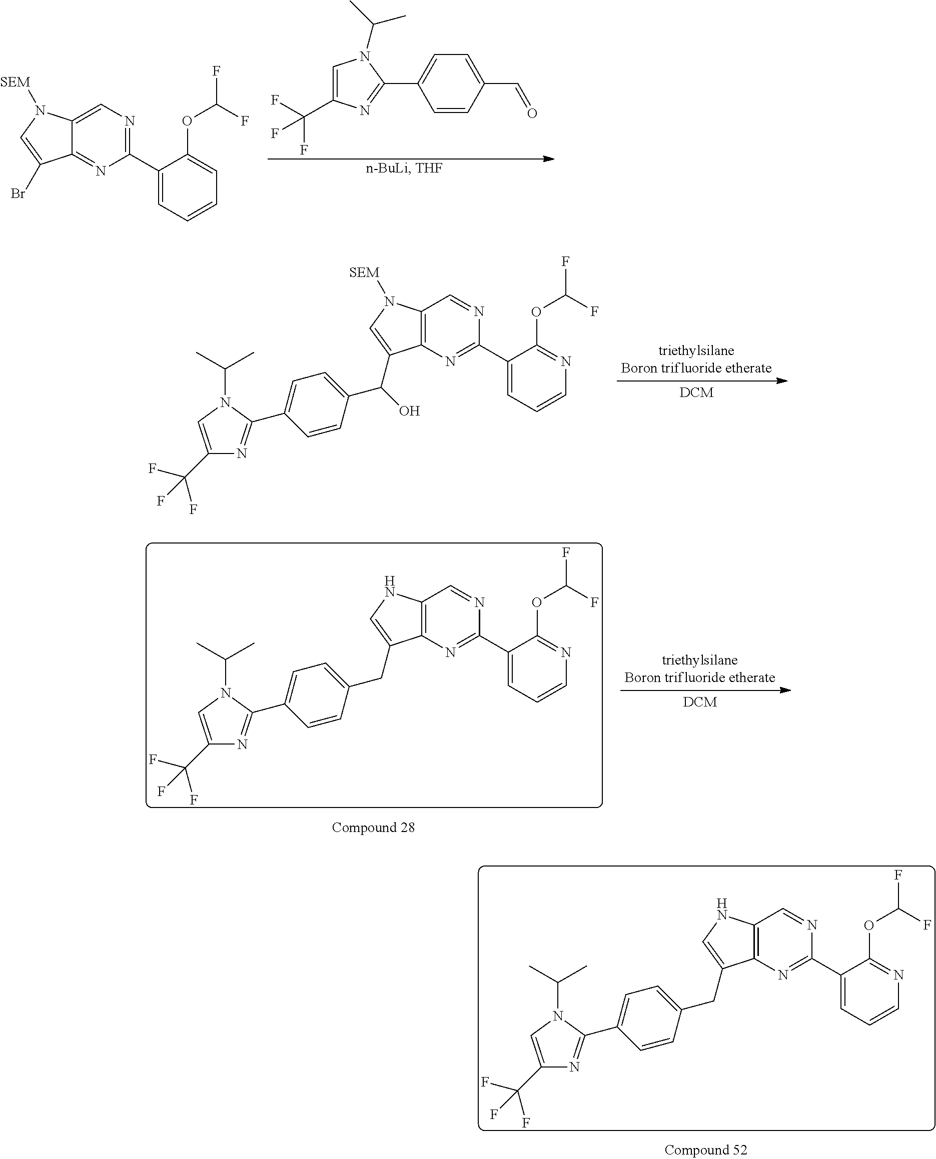

### [2-[2-(difluoromethoxy)-3-pyridyl]-5-(2-trimethylsilylethoxymethyl)pyrrolo[3,2-d]pyrimidin-7-yl]-[4-[1-isopropyl-4-(trifluoromethyl)imidazol-2-yl]phenyl]methanol

To a solution of 2-[[7-bromo-2-[2-(difluoromethoxy)phenyl]pyrrolo[3,2-d]pyrimidin-5-yl]methoxy]ethyl-trimethyl-silane (prepared as above) (100 mg, 212.59 umol) and 4-[1-isopropyl-4-(trifluoromethyl)imidazol-2-yl]benzaldehyde (75.01 mg, 265.74 μmol) in tetrahydrofuran (1 mL) was added n-butyllithium (16.34 mg, 255.11 μmol) at −78° C., and then stirred at 25° C. for 2 hours under N₂ atmosphere. The resulting mixture was quenched by the addition of ammonium chloride (50 mL) at 0° C., then extracted with ethyl acetate (3×20 mL). The combined organic fractions were dried over anhydrous sodium sulfate. After filtration, the filtrate was concentrated under reduced pressure. The residue was purified by silica gel column chromatography, eluted with 1%˜60% ethyl acetate in Petroleum ether to give [2-[2-(difluoromethoxy)-3-pyridyl]-5-(2-trimethylsilylethoxymethyl)pyrrolo[3,2-d]pyrimidin-7-yl]-[4-[1-isopropyl-4-(trifluoromethyl)imidazol-2-yl]phenyl]methanol (80 mg, 118.57 μmol, 55% yield) as a yellow solid. MS: m/z=675.35 [M+H]⁺. ¹H NMR (400 MHz, Chloroform-d) δ 9.11 (s, 1H), 8.45-8.38 (m, 1H), 8.34-8.28 (m, 1H), 7.74-7.67 (m, 2H), 7.67 (t, J=72.8 Hz, 1H), 7.63-7.57 (m, 1H), 7.51-7.42 (m, 2H), 7.33-7.26 (m, 1H), 7.12 (s, 1H), 6.39 (s, 1H), 5.49 (s, 2H), 4.63-4.56 (m, 1H), 3.61-3.47 (m, 2H), 1.50-1.47 (m, 6H), 0.97-0.89 (m, 2H), −0.01 (s, 9H).

### 2-[2-(difluoromethoxy)-3-pyridyl]-7-[[4-[1-isopropyl-4-(trifluoromethyl)imidazol-2-yl]phenyl]ethyl]-5H-pyrrolo[3,2-d]pyrimidine

To a solution of [2-[2-(difluoromethoxy)-3-pyridyl]-5-(2-trimethylsilylethoxymethyl)pyrrolo[3,2-d]pyrimidin-7-yl]-[4-[1-isopropyl-4-(trifluoromethyl)imidazol-2-yl]phenyl]methanol (90 mg, 133.39 μmol) and triethylsilane (310.20 mg, 2.67 mmol, 426.10 μL) in dichloromethane (2 mL) was added boron trifluoride etherate (378.63 mg, 2.67 mmol, 335.07 μL) at 0° C. The resulting mixture was stirred at 25° C. for 16 h under N₂ atmosphere. The resulting mixture was filtered and then concentrated under reduced pressure. The residue was purified by RP-Flash with the following conditions: Column: C18 spherical Column, 20-35 um, 100A, 40 g; Mobile Phase A: Water; Mobile Phase B: MeCN; Flow rate: 50 mL/min; Gradient: 0% B to 0% B in 5 min, 0% B to 60% B in 20 min, 60% B to 95% B in 5 min; Detector: UV 254 & 210 nm; RT: 19 min. The collected fractions were combined, concentrated under reduced pressure and then lyophilized overnight to afford 2-[2-(difluoromethoxy)-3-pyridyl]-7-[[4-[1-isopropyl-4-(trifluoromethyl)imidazol-2-yl]phenyl]methyl]-5H-pyrrolo[3,2-d]pyrimidine (4.7 mg, 8.89 μmol, 7% yield) as an off-white solid. MS: m/z=529.25 [M+H]⁺. ¹H NMR (400 MHz, Methanol-d₄) δ 9.10 (s, 1H), 8.45-8.39 (m, 11H), 8.34-8.27 (m, 11H), 8.04 (s, 1H), 7.94-7.86 (s, 1H), 7.71 (t, J=72.8 Hz, 1H), 7.61-7.51 (m, 2H), 7.50-7.40 (m, 3H), 4.60-4.48 (m, 1H), 4.35 (s, 2H), 1.44 (d, J=6.8 Hz, 6H); ¹⁹F NMR (376 MHz, Methanol-d₄) δ −63.870, −90.138.

### [2-[2-(difluoromethoxy)-3-pyridyl]-5H-pyrrolo[3,2-d]pyrimidin-7-yl]-[4-[1-isopropyl-4-(trifluoromethyl)imidazol-2-yl]phenyl]methanol

To a solution of [2-[2-(difluoromethoxy)-3-pyridyl]-5-(2-trimethylsilylethoxymethyl)pyrrolo[3,2-d]pyrimidin-7-yl]-[4-[1-isopropyl-4-(trifluoromethyl)imidazol-2-yl]phenyl]methanol (15 mg, 22.23 μmol) and triethylsilane (51.70 mg, 444.62 μmol, 71.02 μL) in dichloromethane (0.5 mL) was added boron trifluoride etherate (63.10 mg, 444.62 μmol, 55.84 μL) at 0° C. The resulting mixture was stirred at 25° C. for 16 h under N₂ atmosphere then filtered and concentrated under reduced pressure. The residue was purified by RP-Flash with the following conditions: Column: C18 spherical Column, 20-35 μm, 100A, 40 g; Mobile Phase A: Water, Mobile Phase B: MeCN; Flow rate: 50 mL/min; Gradient: 0% B to 0% B in 5 min, 0% B to 50% B in 20 min, 50% B to 95% B in 10 min; Detector: UV 254 & 210 nm; RT: 25 min. The collected fractions were combined, concentrated under reduced pressure and then lyophilized overnight to afford [2-[2-(difluoromethoxy)-3-pyridyl]-5H-pyrrolo[3,2-d]pyrimidin-7-yl]-[4-[1-isopropyl-4-(trifluoromethyl)imidazol-2-yl]phenyl]methanol (2.7 mg, 4.96 μmol, 22% yield) as an off-white solid. MS: m/z=545.25 [M+H]⁺. ¹H NMR (400 MHz, Methanol-d₄) δ 8.97 (s, 1H), 8.37-8.31 (m, 1H), 8.25-8.18 (m, 1H), 7.90 (s, 1H), 7.86-7.76 (m, 3H), 7.67 (t, J=72.8 Hz, 1H), 7.57-7.46 (m, 2H), 7.41-7.34 (m, 1H), 6.45 (s, 1H), 4.63-4.50 (m, 1H), 1.48-1.41 (m, 6H); ¹⁹F NMR (376 MHz, Methanol-d₄) δ −63.874, −89.880.

### Example 33. (R)-1-[4-[1-methyl-4-(trifluoromethyl)imidazol-2-yl]phenyl]-1-[2-[2-(2,2,2-trifluoroethoxy)-3-pyridyl]-5H-pyrrolo[3,2-d]pyrimidin-7-yl]ethanol (Compound 22) and and (S)-1-[4-[1-methyl-4-(trifluoromethyl)imidazol-2-yl]phenyl]-1-[2-[2-(2,2,2-trifluoroethoxy)-3-pyridyl]-5H-pyrrolo[3,2-d]pyrimidin-7-yl]ethanol and (Compound 74) (Arbitrarily Assigned)

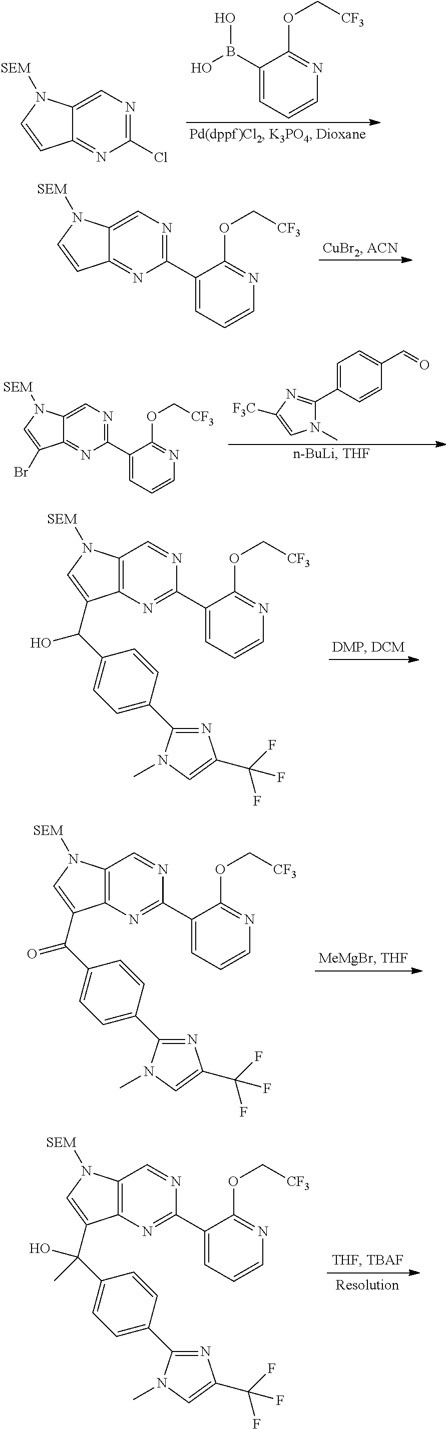

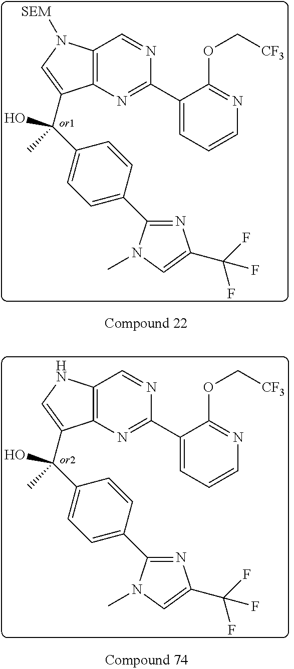 [SMILES: Cn1cc(C(F)(F)F)nc1-c1ccc([C@@](C)(O)c2cn(COCC[Si](C)(C)C)c3cnc(-c4cccnc4OCC(F)(F)F)nc23)cc1 ; Cn1cc(C(F)(F)F)nc1-c1ccc([C@@](C)(O)c2c[nH]c3cnc(-c4cccnc4OCC(F)(F)F)nc23)cc1]

### trimethyl-[2-[[2-[2-(2,2,2-trifluoroethoxy)-3-pyridyl]pyrrolo[3,2-d]pyrimidin-5-yl]methoxy]ethyl]silane

To a solution of 2-[(2-chloropyrrolo[3,2-d]pyrimidin-5-yl)methoxy]ethyl-trimethyl-silane (1 g, 3.52 mmol) and potassium phosphate tribasic anhydrous (1.49 g, 7.03 mmol) in 1,4-dioxane (10 mL) were added [2-(2,2,2-trifluoroethoxy)-3-pyridyl]boronic acid (778.43 mg, 3.52 mmol) and 1,1′-Bis(diphenylphosphino)ferrocene-palladium (II) dichloromethane complex (288.00 mg, 352.51 μmol) at 25° C. under N₂ atmosphere. Then the mixture was heated to 100° C. and stirred for 16 h under N₂ atmosphere. The mixture was cooled down to 25° C. The resulted mixture was diluted by saturated aqueous ammonium chloride (50 mL), extracted by EA (30 mL×2). The organic layer was washed with brine (30 mL×3), dried over anhydrous sodium sulfate. After filtration, the filtrate was concentrated under reduced pressure. The residue was further purified by silica gel column chromatography, eluted with 30%-40% EtOAc in PE to afford trimethyl-[2-[[2-[2-(2,2,2-trifluoroethoxy)-3-pyridyl]pyrrolo[3,2-d]pyrimidin-5-yl]methoxy]ethyl]silane (1 g, 2.36 mmol, 66% yield) as a yellow oil. MS: m/z=425.10 [M+H]⁺. ¹H NMR (400 MHz, Chloroform-d) δ 9.09 (s, 1H), 8.25-8.22 (m, 1H), 8.18-8.16 (m, 1H), 7.61 (d, J=3.2 Hz, 1H), 7.15-7.12 (m, 1H), 6.79 (d, J=3.2 Hz, 11H), 5.60 (s, 2H), 4.93-4.86 (m, 2H), 3.61-3.49 (m, 2H), 0.97-0.93 (m, 2H), 0.00 (s, 9H).

### 2-[[7-bromo-2-[2-(2,2,2-trifluoroethoxy)-3-pyridyl]pyrrolo[3,2-d]pyrimidin-5-yl]methoxy]ethyl-trimethyl-silane

To a solution of trimethyl-[2-[[2-[2-(2,2,2-trifluoroethoxy)-3-pyridyl]pyrrolo[3,2-d]pyrimidin-5-yl]methoxy]ethyl]silane (1 g, 2.36 mmol) in ACN (12 mL) was added copper bromide (1.58 g, 7.07 mol) at 0° C. under N₂ atmosphere. The resulting mixture was stirred at 25° C. for 5 h then 7 M Ammonia in methanol (2 mL) was added and the resulting mixture was stirred at 25° C. for 30 minutes. The resulted mixture was diluted by saturated aqueous ammonium chloride (50 mL), extracted by EA (30 mL×2). The organic layer was washed with brine (30 mL×3), dried over anhydrous sodium sulfate. After filtration, the filtrate was concentrated under reduced pressure. Then was further purified by silica gel column chromatography, eluted with 30%-40% EtOAc in PE to afford 2-[[7-bromo-2-[2-(2,2,2-trifluoroethoxy)-3-pyridyl]pyrrolo[3,2-d]pyrimidin-5-yl]methoxy]ethyl-trimethyl-silane (700 mg, 1.39 mmol, 59% yield) as a yellow oil. MS: m/z=503.00, 505.00 [M+H]⁺. ¹H NMR (400 MHz, Chloroform-d) δ 9.11 (s, 1H), 8.29-8.24 (m, 2H), 7.67 (s, 1H), 7.17-7.14 (m, 1H), 5.58 (s, 2H), 4.93-4.87 (m, 2H), 3.59-3.55 (m, 2H), 0.98-0.93 (m, 2H), 0.00 (s, 9H).

### [4-[1-methyl-4-(trifluoromethyl)imidazol-2-yl]phenyl]-[2-[2-(2,2,2-trifluoroethoxy)-3-pyridyl]-5-(2-trimethylsilylethoxymethyl)pyrrolo[3,2-d]pyrimidin-7-yl]methanol

To a solution of 2-[[7-bromo-2-[2-(2,2,2-trifluoroethoxy)-3-pyridyl]pyrrolo[3,2-d]pyrimidin-5-yl]methoxy]ethyl-trimethyl-silane (300 mg, 595.96 μmol) and 4-[1-methyl-4-(trifluoromethyl)imidazol-2-yl]benzaldehyde (151.50 mg, 595.96 μmol) in THF (1 mL) was added n-butyllithium (0.29 mL, 715.15 μmol, 2.5 M in hexane) at −78° C. under N₂ atmosphere, and the reaction mixture was stirred at −78° C. for 30 min. The resulting solution was naturally warmed up to 25° C., and stirred for 2 h. The resulted mixture was diluted by saturated aqueous ammonium chloride (50 mL), extracted by EA (30 mL×2). The organic layer was washed with brine (30 mL×3), dried over anhydrous sodium sulfate. After filtration, the filtrate was concentrated under reduced pressure. The residue was purified by Prep-TLC (PE:EA=1:3) to afford [4-[1-methyl-4-(trifluoromethyl)imidazol-2-yl]phenyl]-[2-[2-(2,2,2-trifluoroethoxy)-3-pyridyl]-5-(2-trimethylsilylethoxymethyl)pyrrolo[3,2-d]pyrimidin-7-yl]methanol (150 mg, 221.01 μmol, 37% yield) as a yellow oil. MS: m/z=701.35 [M+Na]⁺. ¹H NMR (400 MHz, Chloroform-d) δ 9.10 (s, 1H), 8.40-8.20 (m, 2H), 7.69 (s, 4H), 7.35 (s, 1H), 7.19-7.16 (m, 1H), 7.05-6.98 (m, 1H), 6.41 (s, 1H), 5.50 (s, 2H), 4.97-4.91 (m, 2H), 3.80 (s, 3H), 3.55-3.49 (m, 2H), 1.00-0.88 (m, 2H), 0.00 (s, 9H).

### [4-[1-methyl-4-(trifluoromethyl)imidazol-2-yl]phenyl]-[2-[2-(2,2,2-trifluoroethoxy)-3-pyridyl]-5-(2-trimethylsilylethoxymethyl)pyrrolo[3,2-d]pyrimidin-7-yl]methanone

To a solution of [4-[1-methyl-4-(trifluoromethyl)imidazol-2-yl]phenyl]-[2-[2-(2,2,2-trifluoroethoxy)-3-pyridyl]-5-(2-trimethylsilylethoxymethyl)pyrrolo[3,2-d]pyrimidin-7-yl]methanol (120 mg, 176.81 μmol) in DCM (2 mL) was added Dess-Martin Reagent (149.98 mg, 353.62 μmol) at 0° C. under N₂ atmosphere, and the reaction mixture was stirred for 2 h. The resulted mixture was diluted by NaHCO₃ (50 mL), was extracted by EA (30 mL×2). The organic layer was washed with brine (30 mL×3), dried over anhydrous Na₂SO₄. After filtration, the filtrate was concentrated under reduced pressure. The resulted mixture was purified by Prep-TLC (PE:EA=1:1) to afford [4-[1-methyl-4-(trifluoromethyl)imidazol-2-yl]phenyl]-[2-[2-(2,2,2-trifluoroethoxy)-3-pyridyl]-5-(2-trimethylsilylethoxymethyl)pyrrolo[3,2-d]pyrimidin-7-yl]methanone (80 mg, 118.22 μmol, 66% yield) as a yellow oil. MS: m/z=677.10 [M+H]⁺. ¹H NMR (400 MHz, Chloroform-d) δ crude

### 1-[4-[1-methyl-4-(trifluoromethyl)imidazol-2-yl]phenyl]-1-[2-[2-(2,2,2-trifluoroethoxy)-3-pyridyl]-5-(2-trimethylsilylethoxymethyl)pyrrolo[3,2-d]pyrimidin-7-yl]ethanol

To a solution of [4-[1-methyl-4-(trifluoromethyl)imidazol-2-yl]phenyl]-[2-[2-(2,2,2-trifluoroethoxy)-3-pyridyl]-5-(2-trimethylsilylethoxymethyl)pyrrolo[3,2-d]pyrimidin-7-yl]methanone (64 mg, 94.58 μmol) in THF (1.5 mL) was added methyl magnesium bromide (0.189 mL, 189.16 μmol, 1 M in THF) at 0° C. under N₂ atmosphere. Then the resulting solution was naturally warmed up to 25° C., and stirred for 4 h. The resulted mixture was diluted by aq. NH₄C₁ (50 mL), was extracted by EA (30 mL×2). The organic layer was washed with brine (30 mL×3), dried over anhydrous Na₂SO₄. After filtration, the filtrate was concentrated under reduced pressure. The resulted mixture was diluted by Prep-TLC (PE:EA=1:1) to afford 1-[4-[1-methyl-4-(trifluoromethyl)imidazol-2-yl]phenyl]-1-[2-[2-(2,2,2-trifluoroethoxy)-3-pyridyl]-5-(2-trimethylsilylethoxymethyl)pyrrolo[3,2-d]pyrimidin-7-yl]ethanol (38 mg, 54.86 μmol, 58% yield) as a yellow oil. MS: m/z=693.35 [M+H]⁺. ¹H NMR (400 MHz, Chloroform-d) δ crude

### (R)-1-[4-[1-methyl-4-(trifluoromethyl)imidazol-2-yl]phenyl]-1-[2-[2-(2,2,2-trifluoroethoxy)-3-pyridyl]-5H-pyrrolo[3,2-d]pyrimidin-7-yl]ethanol & (S)-1-[4-[1-methyl-4-(trifluoromethyl)imidazol-2-yl]phenyl]-1-[2-[2-(2,2,2-trifluoroethoxy)-3-pyridyl]-5H-pyrrolo[3,2-d]pyrimidin-7-yl]ethanol

To a solution of 1-[4-[1-methyl-4-(trifluoromethyl)imidazol-2-yl]phenyl]-1-[2-[2-(2,2,2-trifluoroethoxy)-3-pyridyl]-5-(2-trimethylsilylethoxymethyl)pyrrolo[3,2-d]pyrimidin-7-yl]ethanol (30 mg, 43.31 μmol) in THF (3 mL) was added tetrabutylammonium fluoride (0.17 mL, 173.23 μmol, 1 M in THF) at the 60° C. then the resulting mixture was stirred for 1 h. The resulted mixture was purified by Prep-TLC (DCM:MeOH=10:1), then was further purified by RP-Flash chromatography with the following conditions: Column: C18 spherical, 20-35 um, 100A, 40 g; Mobile Phase A: 10 mM aq. NH₄HCO₁, Mobile Phase B: MeCN; Flow rate: 40 mL/min; Gradient: 20% B to 95% B in 25 min; Detector: UV 254 & 220 nm; RT: 20 min to afford the mixture of two isomers (18 mg). The 18 mg mixture product was separated by Prep-Chiral-HPLC with the following conditions: Column: CHIRALPAK IE, 2×25 cm, 5 um; Mobile Phase A: Hex (0.5% 2 M NH₃-MeOH), Mobile Phase B: IPA; Flow rate: 20 mL/min; Gradient: 15% B to 15% B in 19 min; Detector: UV 220/254 nm; RT1: 13.155 min; RT2: 16.248 min; Injection Volume: 0.6 mL; Number Of Runs: 3. The collected fractions were combined and concentrated under reduced pressure to afford (R)-1-[4-[1-methyl-4-(trifluoromethyl)imidazol-2-yl]phenyl]-1-[2-[2-(2,2,2-trifluoroethoxy)-3-pyridyl]-5H-pyrrolo[3,2-d]pyrimidin-7-yl]ethanol (3.7 mg, 6.58 μmol, 4% yield, RT1: 13.155 min) as a white solid. MS: m/z=563.10 [M+H]⁺. ¹H NMR (400 MHz, Chloroform-d) δ 10.78 (s, 1H), 8.95 (s, 1H), 8.27-8.21 (m, 2H), 7.65-7.63 (m, 2H), 7.58-7.56 (m, 2H), 7.39 (s, 1H), 7.13-7.10 (m, 1H), 6.99 (s, 1H), 5.69 (s, 1H), 4.95-4.79 (m, 2H), 3.80 (s, 3H), 1.97 (s, 3H); ¹⁹F NMR (376 MHz, Chloroform-d) δ −62.342, −73.225.

and (S)-1-[4-[1-methyl-4-(trifluoromethyl)imidazol-2-yl]phenyl]-1-[2-[2-(2,2,2-trifluoroethoxy)-3-pyridyl]-5H-pyrrolo[3,2-d]pyrimidin-7-yl]ethanol (3.4 mg, 6.04 μmol, 3% yield) as white solid. Stereochemistry arbitrarily assigned. MS: m/z=563.10 [M+H]⁺. ¹H NMR (400 MHz, Chloroform-d) δ 10.71 (s, 1H), 8.95 (s, 1H), 8.27-8.21 (m, 2H), 7.65-7.63 (m, 2H), 7.58-7.54 (m, 2H), 7.39 (s, 1H), 7.13-7.10 (m, 1H), 7.00 (s, 1H), 5.68 (s, 1H), 4.95-4.79 (m, 2H), 3.81 (s, 3H), 1.97 (s, 3H); ¹⁹F NMR (376 MHz, Chloroform-d) δ −62.355, −73.224.

### Example 34. Synthesis of 2-(2-methoxy-3-pyridyl)-7-[1-[4-[1-methyl-4-(trifluoromethyl)imidazol-2-yl]phenyl]cyclopropyl]-5H-pyrrolo[3,2-d]pyrimidine (Compound 8)

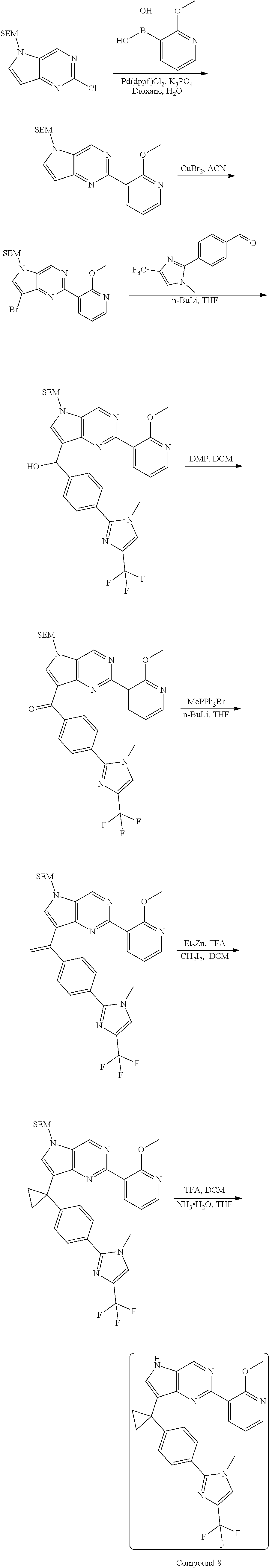

### 2-[[2-(2-methoxy-3-pyridyl)pyrrolo[3,2-d]pyrimidin-5-yl]methoxy]ethyl-trimethyl-silane

To a solution of 2-[(2-chloropyrrolo[3,2-d]pyrimidin-5-yl)methoxy]ethyl-trimethyl-silane (1.5 g, 5.28 mmol) and (2-methoxy-3-pyridyl)boronic acid (1.21 g, 7.93 mmol) in dioxane (15 mL) and water (3 mL) were added potassium phosphate (3.37 g, 15.85 mmol) and 1,1′-Bis(diphenylphosphino)ferrocene-palladium(II)dichloride dichloromethane complex (432.65 mg, 528.49 μmol). The resulting mixture was stirred for 16 h at 100° C. under nitrogen atmosphere then diluted with water (100 mL). The resulting mixture was extracted with EA (3×100 mL). The combined organic layers were dried over anhydrous sodium sulfate. After filtration, the filtrate was concentrated under reduced pressure. The residue was purified by silica gel column chromatography, eluted with EA in PE (0%-49%) to afford 2-[[2-(2-methoxy-3-pyridyl)pyrrolo[3,2-d]pyrimidin-5-yl]methoxy]ethyl-trimethyl-silane (1.68 g, 4.71 mmol, 89% yield) as a yellow solid. MS: m/z=357.10 [M+H]⁺. ¹H NMR (400 MHz, Chloroform-d) δ 9.13 (s, 1H), 8.32-8.26 (m, 1H), 8.14-8.07 (m, 1H), 7.61 (d, J=3.2 Hz, 1H), 7.09-7.01 (m, 1H), 6.81 (d, J=3.2 Hz, 1H), 5.59 (s, 2H), 4.06 (s, 3H), 3.58-3.49 (m, 2H), 1.00-0.89 (m, 2H), 0.00 (s, 9H).

### 2-[[7-bromo-2-(2-methoxy-3-pyridyl)pyrrolo[3,2-d]pyrimidin-5-yl]methoxy]ethyl-trimethyl-silane

To a stirred solution of 2-[[2-(2-methoxy-3-pyridyl)pyrrolo[3,2-d]pyrimidin-5-yl]methoxy]ethyl-trimethyl-silane (2.09 g, 5.86 mmol) in ACN (40 mL) was added copper bromide (3.93 g, 17.59 mmol) at room temperature. The resulting mixture was stirred for 16 h at room temperature then quenched with ammonia (5 mL, 7 M in Methanol). The resulting mixture was diluted with water (100 mL) and extracted with EA (3×100 mL). The combined organic layers were dried over anhydrous sodium sulfate. After filtration, the filtrate was concentrated under reduced pressure. The residue was purified by silica gel column chromatography, eluted with EA in PE (0%-49%) to afford 2-[[7-bromo-2-(2-methoxy-3-pyridyl)pyrrolo[3,2-d]pyrimidin-5-yl]methoxy]ethyl-trimethyl-silane (2 g, 4.59 mmol, 78% yield) as a yellow solid. MS: m/z=435.10 [M+H]⁺. ¹H NMR (400 MHz, Chloroform-d) δ 9.12 (s, 1H), 8.32-8.26 (m, 1H), 8.20-8.13 (m, 1H), 7.64 (s, 1H), 7.09-7.01 (m, 1H), 5.57 (s, 2H), 4.06 (s, 3H), 3.60-3.51 (m, 2H), 1.02-0.90 (m, 2H), 0.00 (s, 9H).

### [2-(2-methoxy-3-pyridyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol

To a solution of 2-[[7-bromo-2-(2-methoxy-3-pyridyl)pyrrolo[3,2-d]pyrimidin-5-yl]methoxy]ethyl-trimethyl-silane (0.790 g, 1.81 mmol) in THF (3 mL), n-butyllithium solution (2.5 M in hexane, 0.55 mL, 1.38 mmol) was added dropwise at −70° C. under nitrogen atmosphere. The reaction mixture was stirred at −70° C. for 10 mins., then a solution of 4-[1-methyl-4-(trifluoromethyl)imidazol-2-yl]benzaldehyde (507.38 mg, 2.00 mmol) in 3 mL THF was added dropwise. The resulting mixture was stirred for another 30 mins then quenched with saturated aqueous ammonium chloride (100 mL) at room temperature. The resulting mixture was extracted with EA (3×100 mL). The combined organic layers were dried over anhydrous sodium sulfate. After filtration, the filtrate was concentrated under reduced pressure. The residue was purified by silica gel column chromatography, eluted with EA in PE (0%-97%) to afford [2-(2-methoxy-3-pyridyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol (0.34 g, 556.74 μmol, 30% yield) as a yellow solid. MS: m/z=611.15 [M+H]⁺.

### [2-(2-methoxy-3-pyridyl)-5-(2-trimethylsilylethoxymethyl)pyrrolo[3,2-d]pyrimidin-7-yl]-[4-[1-methyl-4-(trifluoromethyl)imidazol-2-yl]phenyl]methanone

To a stirring solution of [2-(2-methoxy-3-pyridyl)-5-(2-trimethylsilylethoxymethyl)pyrrolo[3,2-d]pyrimidin-7-yl]-[4-[1-methyl-4-(trifluoromethyl)imidazol-2-yl]phenyl]methanol (0.341 g, 558.37 μmol) in DCM (3 mL) was added Dess-Martin Reagent (473.66 mg, 1.12 mmol) at room temperature. The resulting mixture was stirred for 3 h at room temperature then diluted with water (50 mL). The resulting mixture was extracted with DCM (3×50 mL). The combined organic layers were dried over anhydrous sodium sulfate. After filtration, the filtrate was concentrated under reduced pressure. The residue was purified by silica gel column chromatography, eluted with EA in PE (40%) to afford [2-(2-methoxy-3-pyridyl)-5-(2-trimethylsilylethoxymethyl)pyrrolo[3,2-d]pyrimidin-7-yl]-[4-[1-methyl-4-(trifluoromethyl)imidazol-2-yl]phenyl]methanone (0.29 g, 476.44 umol, 85% yield) as yellow solid. MS: m/z=609.15 [M+H]⁺. ¹H NMR (400 MHz, Methanol-d₄) δ 9.27 (s, 1H), 8.66 (s, 1H), 8.30-8.21 (m, 1H), 8.21-8.10 (m, 2H), 8.11-8.06 (m, 1H), 7.92-7.82 (m, 2H), 7.81-7.67 (m, 1H), 7.14-7.04 (m, 1H), 5.85 (s, 2H), 3.97 (s, 3H), 3.87 (s, 3H), 3.73-3.62 (m, 2H), 1.03-0.86 (m, 2H), −0.10 (s, 9H).

### (2-methoxy-3-pyridyl)-7-[1-[4-[1-methyl-4-(trifluoromethyl)imidazol-2-yl]phenyl]vinyl]pyrrolo[3,2-d]pyrimidin-5-yl]methoxy]ethyl-trimethyl-silane

To a solution of methyltriphenylphosphonium bromide (250 mg, 0.70 mmol) in dry THF (5 mL), n-butyl lithium (0.28 mL, 0.70 umol, 2.5 M in hexane) was added dropwise at −70° C. After the reaction mixture was stirred for 20 min under nitrogen, [2-(2-methoxy-3-pyridyl)-5-(2-trimethylsilylethoxymethyl)pyrrolo[3,2-d]pyrimidin-7-yl]-[4-[-methyl-4-(trifluoromethyl)imidazol-2-yl]phenyl]methanone (212 mg, 0.35 mmol) in dry THF (1 mL) was added dropwise. After the reaction mixture was stirred at 0° C. for 1 h under nitrogen, saturated aqueous ammonium chloride (50 mL) was added. The mixture solution was extracted with ethyl acetate (3×20 mL). The combined organic layers were washed with brine (3×10 mL), dried over anhydrous sodium sulfate. After filtration, the filtrate was concentrated under reduced pressure. The residue was purified by silica gel column chromatography, eluted with EA in PE (0%-49%) to afford 2-[[2-(2-methoxy-3-pyridyl)-7-[1-[4-[1-methyl-4-(trifluoromethyl)imidazol-2-yl]phenyl]vinyl]pyrrolo[3,2-d]pyrimidin-5-yl]methoxy]ethyl-trimethyl-silane (80 mg, 0.132 mmol, 19% yield) as a yellow solid. MS: m/z=607.2 [M+H]⁺.

### 2-[[2-(2-methoxy-3-pyridyl)-7-[1-[4-[1-methyl-4-(trifluoromethyl)imidazol-2-yl]phenyl]cyclopropyl]pyrrolo[3,2-d]pyrimidin-5-yl]methoxy]ethyl-trimethyl-silane

Diethylzinc (0.26 mL, 263.72 μmol, 1 M in hexane) was added to freshly distilled DCM (0.5 mL) under N₂. The resulting solution was cooled in an ice bath and a solution of trifluoroacetic acid (32.32 mg, 283.50 μmol) in DCM (0.5 mL) was then added dropwise. After the resulting mixture was stirred for 20 min at 0° C., a solution of diiodomethane (73.1 mg, 272.95 μmol) in DCM (0.5 mL) was added. After stirring for another 20 min at 0° C., a solution of 2-[[2-(2-methoxy-3-pyridyl)-7-[1-[4-[1-methyl-4-(trifluoromethyl)imidazol-2-yl]phenyl]vinyl]pyrrolo[3,2-d]pyrimidin-5-yl]methoxy]ethyl-trimethyl-silane (0.08 g, 131.86 μmol) in DCM (1 mL) was added. The reaction mixture was stirred at 0° C. for 6 h at room temperature then quenched by saturated aqueous ammonium chloride (20 mL). The resulting solution was extracted with ethyl acetate (3×10 mL). The combined organic layers were washed with brine (3×10 mL), dried over anhydrous sodium sulfate. After filtration, the filtrate was concentrated under reduced pressure. The residue was purified by Prep-TLC (CH₂Cl₂: MeOH=10:1) to afford 30 mg crude. The crude was further purified by reverse phase chromatography with the following conditions: Column: Spherical C18, 20-40 um, 40 g; Mobile Phase A: Water (10 mM aq. NH₄HCOs), Mobile Phase B: ACN; Flow rate: 40 mL/min; Gradient (B %): 5% hold 5 min, 5%-60% within 20 min, 60% hold 5 min, 60%-95% within 5 min; Detector: UV 254 & 220 nm; RT: 27 min. The collected fractions were combined and concentrated under reduced pressure to afford 2-[[2-(2-methoxy-3-pyridyl)-7-[1-[4-[1-methyl-4-(trifluoromethyl)imidazol-2-yl]phenyl]cyclopropyl]pyrrolo[3,2-d]pyrimidin-5-yl]methoxy]ethyl-trimethyl-silane (18 mg, 29.00 μmol, 21% yield) as a light yellow solid. MS: m/z=621.25 [M+H]⁺.

### 2-(2-methoxy-3-pyridyl-1)-7-[1-[4-[1-methyl-4-(trifluoromethyl)imidazol-2-yl]phenyl]cyclopropyl]-5H-pyrrolo[3,2-d]pyrimidine

To a solution of 2-[[2-(2-methoxy-3-pyridyl)-7-[1-[4-[1-methyl-4-(trifluoromethyl)imidazol-2-yl]phenyl]cyclopropyl]pyrrolo[3,2-d]pyrimidin-5-yl]methoxy]ethyl-trimethyl-silane (18 mg, 29.00 umol) in DCM (0.5 mL) was added 2,2,2-trifluoroacetic acid (3.31 mg, 29.00 μmol, 2.23 μL) at 0° C. under N₂ atmosphere. The resulting mixture was stirred for 2 h at room temperature then concentrated under reduced pressure. The residue dissolved in THF (0.5 mL) and ammonium hydroxide (28%, 0.5 mL) was added. The resulting mixture was stirred for another 1 h at room temperature then concentrated under reduced pressure. The residue was dissolved in brine (50 mL) and extracted with EA (3×50 mL). The combined organic phase was dried over anhydrous sodium sulfate. After filtration, the filtrate was concentrated under reduced pressure. The crude product was purified by reverse phase chromatography with the following conditions: Column: Spherical C18, 20-40 um, 40 g; Mobile Phase A: 10 mM aq. NH₄HCO₃, Mobile Phase B: ACN; Flow rate: 40 mL/min; Gradient (B %): 0% hold 5 min, 5%-20% within 5 min; 20%-95% within 2 min, 95% hold 5 min; Detector: UV 254 & 220 nm; RT: 5 min. The fractions were collected, concentrated under reduced pressure and then lyophilized overnight to give 2-(2-methoxy-3-pyridyl)-7-[1-[4-[1-methyl-4-(trifluoromethyl)imidazol-2-yl]phenyl]cyclopropyl]-5H-pyrrolo[3,2-d]pyrimidine as a white solid. MS: m/z=491.15 [M+H]⁺. ¹H NMR (400 MHz, Chloroform-d) δ 9.16 (s, 1H), 8.29 (d, J=3.2 Hz, 1H), 8.24 (d, J=7.6 Hz, 1H), 7.57 (d, J=8.4 Hz, 2H), 7.51 (d, J=8.4 Hz, 2H), 7.34-7.32 (m, 2H), 7.11-7.04 (m, 1H), 4.12 (s, 3H), 3.78 (s, 3H), 1.87-1.85 (m, 2H), 1.42-1.39 (m, 2H); ¹⁹F NMR (376 MHz, Chloroform-d) δ −62.644.

### Example 35. Synthesis of 2-(4-cyclopropyl-6-methoxy-pyrimidin-5-yl)-7-[1-[4-[1-methyl-4-(trifluoromethyl)imidazol-2-yl]phenyl]cyclopropyl]-5H-pyrrolo[3,2-d]pyrimidine (Compound 47)

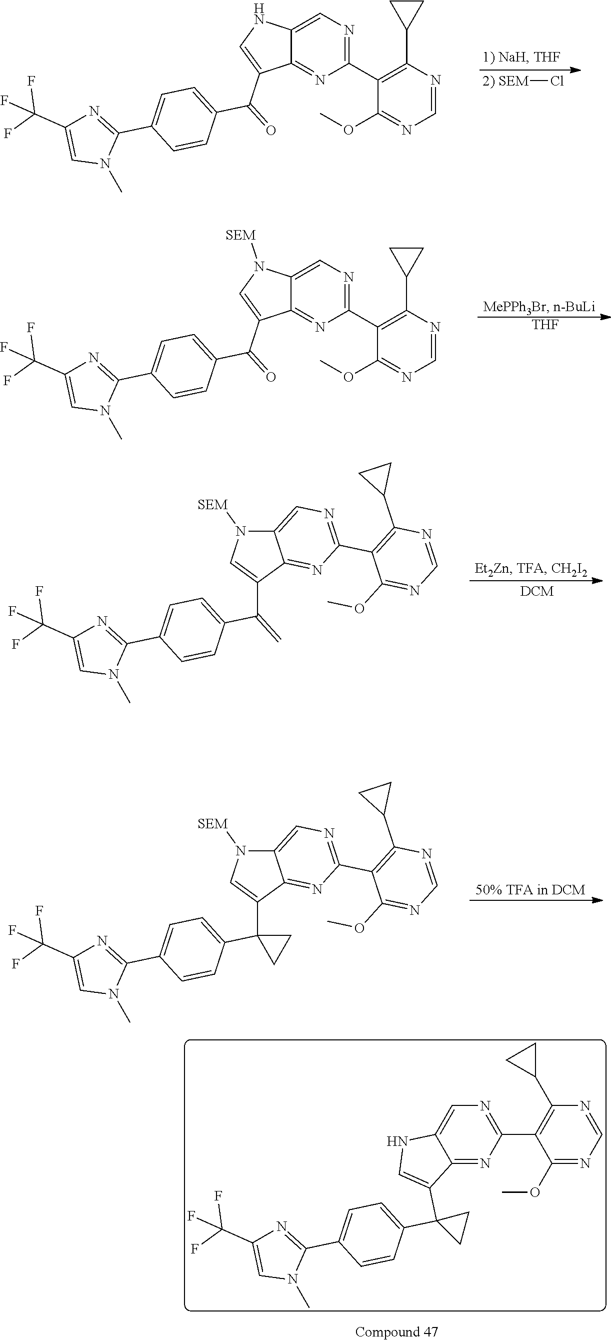

### (2-(4-cyclopropyl-6-methoxypyrimidin-5-yl)-5-((2-(trimethylsilyl)ethoxy)methyl)-5H-pyrrolo[3,2-d]pyrimidin-7-yl)(4-(1-methyl-4-(trifluoromethyl)-1H-imidazol-2-yl)phenyl)methanone

To a solution of [2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanone (400 mg, 770.01 umol) in THF (5 mL) was added sodium hydride (36.66 mg, 924.01 umol, 60% in oil) at 0° C. The resulting mixture was stirred for 1 hour at 0° C. then 2-(trimethylsilyl)ethoxymethyl chloride (141.21 mg, 847.01 μmol, 149.91 μL) was added to the above mixture at 0° C. The resulting mixture was stirred for 2 hours at 25° C. then quenched by saturated aqueous ammonium chloride (30 mL), was extracted by EA (30 mL×2). The organic layer was washed with brine (20 mL×2), dried over anhydrous Na₂SO₄. After filtration, the filtrate was concentrated under reduced pressure. The residue was purified by Prep-TLC (DCM:MeOH=10:1) to afford [2-(4-cyclopropyl-6-methoxy-pyrimidin-5-yl)-5-(2-trimethylsilylethoxymethyl)pyrrolo[3,2-d]pyrimidin-7-yl]-[4-[1-methyl-4-(trifluoromethyl)imidazol-2-yl]phenyl]methanone (370 mg, 569.46 μmol, 74% yield) as a yellow solid. MS: m/z=650.15 [M+H]⁺. ¹H NMR (400 MHz, Chloroform-d) δ 9.25 (s, 1H), 8.64 (s. 1H), 8.25 (s, 1H), 8.17-8.14 (m, 2H), 7.77-7.74 (m, 2H), 7.38-7.37 (m, 1H), 5.69 (s, 2H), 3.93 (s, 3H), 3.82 (s, 3H), 3.68-3.64 (m, 2H), 1.24-1.21 (m, 1H), 1.03-1.01 (m, 2H), 0.99-0.88 (m, 4H), 0.02 (s, 9H). ¹⁹F NMR (376 MHz, Chloroform-d) δ −62.763.

### 2-(4-cyclopropyl-6-methoxypyrimidin-5-yl)-7-(1-(4-(1-methyl-4-(trifluoromethyl)-1H-imidazol-2-yl)phenyl)vinyl)-5-((2-(trimethylsilyl)ethoxy)methyl)-5H-pyrrolo[3,2-d]pyrimidine

To a solution of methyltriphenylphosphonium bromide (549.80 mg, 1.54 mmol) in THF (3 mL) was added butyllithium (0.62 mL, 1.54 mmol, 2.5 M in hexane) at 0° C. under nitrogen atmosphere. The solution was stirred at 0° C. for 1 hour. Then [2-(4-cyclopropyl-6-methoxy-pyrimidin-5-yl)-5-(2-trimethylsilylethoxymethyl)pyrrolo[3,2-d]pyrimidin-7-yl]-[4-[1-methy-4-(trifluoromethyl)imidazol-2-yl]phenyl]methanone (500 mg, 769.54 μmol) was added to the above mixture at 0° C. The resulting solution was stirred for 2 hours at 25° C. then quenched by saturated aqueous ammonium chloride (20 mL), was extracted by EA (30 mL×2). The organic layer was washed with brine (20 mL×2), dried over anhydrous Na₂SO₄. After filtration, the filtrate was concentrated under reduced pressure. The residue was purified by silica gel column chromatography, eluted with 40%-60% EtOAc in PE to afford 2-[[2-(4-cyclopropyl-6-methoxy-pyrimidin-5-yl)-7-[1-[4-[1-methyl-4-(trifluoromethyl)imidazol-2-yl]phenyl]vinyl]pyrrolo[3,2-d]pyrimidin-5-yl]methoxy]ethyl-trimethyl-silane (170 mg, 262.44 μmol, 34% yield) as a yellow solid. MS: m/z=648.35 [M+H]⁺. ¹H NMR (400 MHz, Chloroform-<d) δ 9.18 (s, 1H), 8.71 (s, 1H), 7.70 (d, J=8.0 Hz, 2H), 7.60 (d, J=8.0 Hz, 2H), 7.36 (d, J=8.8 Hz, 2H), 6.68 (s, 1H), 5.61 (s, 1H), 5.53 (s, 2H), 3.97 (s, 3H), 3.86 (s, 3H), 3.61-3.53 (m, 2H), 1.93-1.89 (m, 1H), 0.98-0.92 (m, 6H), 0.00 (s, 9H). ¹⁹F NMR (376 MHz, Chloroform-d) δ −62.752.

### 2-(4-cyclopropyl-6-methoxypyrimidin-5-yl)-7-(1-(4-(1-methyl-4-(trifluoromethyl)-1H-imidazol-2-yl)phenyl)cyclopropyl)-5-((2-(trimethylsilyl)ethoxy)methyl)-5H-pyrrolo[3,2-d]pyrimidine

To a solution of diethylzinc (0.7 mL, 5.67 μmol, 1 M solution in hexane) in DCM (1 mL) was added 2,2,2-trifluoroacetic acid (98.57 mg, 864.51 μmol, 66.60 μL) at 0° C. After the resulting solution was stirred for 0.5 hours at 0° C., iodomethane (210.36 mg, 1.48 mmol, 92.26 μL) was added and the resulting mixture was stirred for 0.5 hours at 0° C. Then 2-[[2-(4-cyclopropyl-6-methoxy-pyrimidin-5-yl)-7-[1-[4-[1-methyl-4-(trifluoromethyl)imidazol-2-yl]phenyl]vinyl]pyrrolo[3,2-d]pyrimidin-5-yl]methoxy]ethyl-trimethyl-silane (160 mg, 247.00 μmol) was added to at 0° C. The resulting solution was stirred for 16 hours at 0° C., and then naturally warmed up to 25° C. The resulted mixture was quenched by saturated aqueous ammonium chloride (20 mL), was extracted by EA (30 mL×2). The organic layer was washed with brine (30 mL×3), dried over anhydrous Na₂SO₄. After filtration, the filtrate was concentrated under reduced pressure. The residue was purified by Prep-TLC (DCM:MeOH=13:1) to afford 2-[[2-(4-cyclopropyl-6-methoxy-pyrimidin-5-yl)-7-[1-[4-[1-methyl-4-(trifluoromethyl)imidazol-2-yl]phenyl]cyclopropyl]pyrrolo[3,2-d]pyrimidin-5-yl]methoxy]ethyl-trimethyl-silane (10 mg, 15.11 μmol, 6% yield, crude product) as a brown solid, which was directly used in the next step directly without purification.

### 2-(4-cyclopropyl-6-methoxypyrimidin-5-yl)-7-(1-(4-(1-methyl-4-(trifluoromethyl)-1H-imidazol-2-yl)phenyl)cyclopropyl)-5H-pyrrolo[3,2-d]pyrimidine

The solution of 2-[[2-(4-cyclopropyl-6-methoxy-pyrimidin-5-yl)-7-[1-[4-[1-methyl-4-(trifluoromethyl)imidazol-2-yl]phenyl]cyclopropyl]pyrrolo[3,2-d]pyrimidin-5-yl]methoxy]ethyl-trimethyl-silane (10 mg, 15.11 μmol) in TFA (1 mL) and DCM (1 mL) was stirred for 2 hours at 25° C. The resulting solution was concentrated under reduced pressure then THF (1 mL) and ammonium hydroxide (1 mL) were added and the resulting solution was stirred for 10 min at 25° C. The resulted mixture was concentrated under reduced pressure. The residue was purified by Prep-TLC to give˜3 mg crude product, the crude product was further purified by Prep-HPLC: Column: XBridge Prep OBD C18 Column, 19×250 mm, 5 μm; Mobile Phase A: Water (10 mM aq. NH₄HCO₃), Mobile Phase B: MeOH; Flow rate: 25 mL/min; Gradient: 60% B to 60% B in 14 min, Detector: UV 254 & 210 nm; RT=7.6 min) to afford 2-(4-cyclopropyl-6-methoxy-pyrimidin-5-yl)-7-[1-[4-[1-methyl-4-(trifluoromethyl)imidazol-2-yl]phenyl]cyclopropyl]-5H-pyrrolo[3,2-d]pyrimidine (1.9 mg, 3.57 μmol, 24% yield) as an off-white solid. MS: m/z=532.20 [M+H]⁺; ¹H NMR (400 MHz, Chloroform-d) 8.97 (s, 1H), 8.61 (s, 1H), 7.86 (s, 1H), 7.67 (s, 1H), 7.53-7.51 (m, 2H), 7.43-7.41 (m, 2H), 3.91 (s, 3H), 3.75 (s, 3H), 1.67-1.62 (m, 1H), 1.58-1.55 (m, 2H), 1.46-1.44 (m, 2H), 1.14-1.10 (m, 2H), 0.87-0.82 (m, 2H); ¹⁹F NMR (376 MHz, Chloroform-d) δ −63.942.

### Example 36. Synthesis of 5-(2-isopropylphenyl)-3-[[1-[1-methyl-4-(trifluoromethyl)imidazol-2-yl]-4-piperidyl]methyl]-1H-pyrrolo[2,3-c]pyridine (Compound 85)

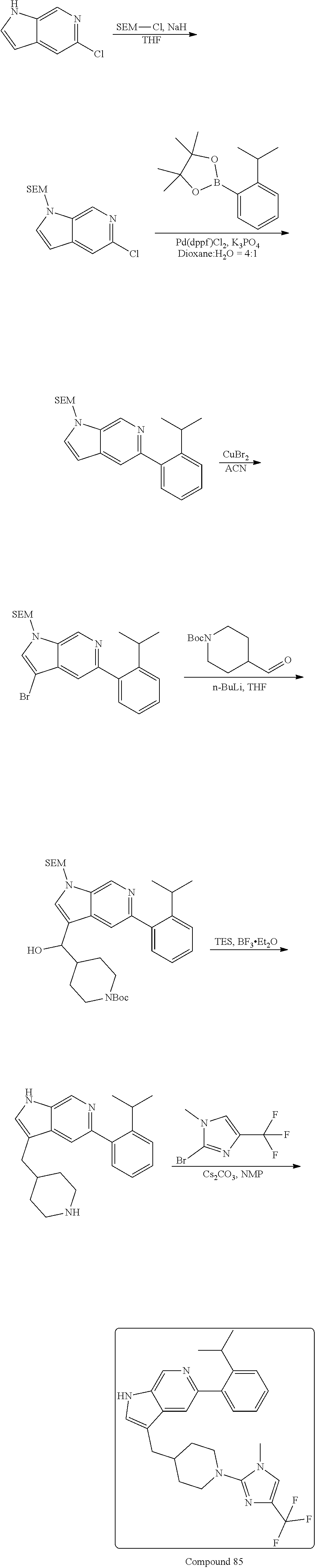

### 2-[(5-chloropyrrolo[2,3-c]pyridin-1 yl)methoxy]ethyl-trimethyl-silane

To a solution of 5-chloro-1H-pyrrolo[2,3-c]pyridine (1.5 g, 9.83 mmol) in THF (20 mL) sodium hydride (60% dispersion in mineral oil, 283.01 mg, 11.79 mmol) was added slowly at 0° C. The resulting mixture was stirred at 0 TC for 1 h, then 2-(chloromethoxy)ethyl-trimethyl-silane (1.97 g, 11.80 mmol, 2.09 mL) was added dropwise at 0 TC. The resulting mixture was stirred at 0° C. for 4 hrs. Then the mixture was diluted with saturated aqueous ammonium chloride (100) mL), extracted with ethyl acetate (3×150 mL). The combined organic phase was washed with brine (300 mL), dried over anhydrous sodium sulfate, the filtrate was concentrated under reduced pressure. The residue was purified by silica gel column chromatography, eluted with 0%-30% EtOAc in PE to afford 2-[(5-chloropyrrolo[2,3-c]pyridin-1-yl)methoxy]ethyl-trimethyl-silane (2.5 g, 8.84 mmol, 89% yield) as a light yellow solid. MS: m/z=283.05 [M+H]⁺. ¹H NMR (400 MHz, Chloroform-d) δ 8.69 (s, 1H), 7.57 (s, 1H), 7.39 (d, J=3.2 Hz, 1H), 6.52 (d, J=3.2, 1H), 5.55 (s, 2H), 3.55-3.40 (m, 2H), 0.98-0.84 (m, 2H), −0.04 (s, 9H).

### 2-[[5-(2-isopropylphenyl)pyrrolo[2,3-c]pyridin-1-yl]methoxy]ethyl-trimethyl-silane

To a solution of 2-(2-isopropylphenyl)-4,4,5,5-tetramethyl-1,3,2-dioxaborolane (3.26 g, 13.26 mmol) and 2-[(5-chloropyrrolo[2,3-c]pyridin-1-yl)methoxy]ethyl-trimethyl-silane (2.5 g, 8.84 mmol) in Dioxane (30 mL) and H₂O (7.5 mL) were added 1,1′-Bis(Diphenylphosphino)ferrocene-palladium(II) dichloride dichloromethane complex (721.82 mg, 883.89 umol) and potassium phosphate (3.75 g, 17.68 mmol). The resulting mixture was stirred for 16 hrs at 100° C. under nitrogen atmosphere. The resulting mixture was diluted with water (500 mL) and extracted with ethyl acetate (3×250 mL). The combined organic extracts were washed with saturated aqueous sodium chloride (300 mL), dried over anhydrous sodium sulfate and concentrated under reduced pressure. The residue was purified by silica gel column chromatography, eluted with 0%-30% EtOAc in PE to afford 2-[[5-(2-isopropylphenyl)pyrrolo[2,3-c]pyridin-1-yl]methoxy]ethyl-trimethyl-silane (2.2 g, 6.00 mmol, 68% yield) as a brown oil. MS: m/z=367.10 [M+H]⁺. ¹H NMR (400 MHz, Chloroform-d) δ 8.91 (s, 1H), 8.59 (s, 1H), 7.50 (s, 1H), 7.38-7.26 (m, 4H), 6.42 (dd, J=3.2, 0.8 Hz, 1H), 5.45 (s, 2H), 3.56-3.35 (m, 2H), 3.13 (d, J=8.4 Hz, 1H), 1.11 (d, J=6.8 Hz, 6H), 0.95-0.78 (m, 2H), −0.12 (m, 9H).

### 2-[[3-bromo-5-(2-isopropylphenyl)pyrrolo[2,3-c]pyridin-1-yl]methoxy]ethyl-trimethyl-silane

To a solution of 2-[[5-(2-isopropylphenyl)pyrrolo[2,3-c]pyridin-1-yl]methoxy]ethyl-trimethyl-silane (2.2 g, 6.00 mmol) in acetonitrile (50 mL) was added cupric bromide (4.02 g, 18.00 mmol) slowly at 0° C. The resulting mixture was stirred at 0° C. for 4 h. Then the mixture was diluted with aq. NH₃H₂O (20 mL), extracted with ethyl acetate (3×50 mL). The combined organic phase was washed with brine (100 mL), dried over anhydrous sodium sulfate. The filtrate was concentrated under reduced pressure and the residue was purified by silica gel column chromatography, eluted with 0%-30% EtOAc in PE to afford 2-[[3-bromo-5-(2-isopropylphenyl)pyrrolo[2,3-c]pyridin-1-yl]methoxy]ethyl-trimethyl-silane (1.8 g, 4.04 mmol, 67% yield) as a light yellow solid. MS: m/z=445.10, 447.10 [M+H]⁺. ¹H NMR (400 MHz, Chloroform-d) δ 9.00 (s, 1H), 8.68 (s, 1H), 7.59-7.51 (m, 1H), 7.45 (d, J=7.2 Hz, 1H), 7.45-7.34 (m, 3H), 5.52 (s, 2H), 3.56-3.52 (m, 2H), 3.23-3.15 (m, 1H), 1.22 (d, J=6.8 Hz, 6H), 1.03-0.88 (m, 2H), 0.09 (s, 9H).

### tert-butyl 4-[hydroxy-[5-(2-isopropylphenyl)-1-(2-trimethylsilylethoxymethyl)pyrrolo[2,3-c]pyridin-3-yl]methyl]piperidine-1-carboxylate

To a solution of 2-[[3-bromo-5-(2-isopropylphenyl)pyrrolo[2,3-c]pyridin-1-yl]methoxy]ethyl-trimethyl-silane (1.1 g, 2.47 mmol) in THF (10 mL), n-Butyllithium (2.5 M in hexane, 2.47 mmol, 0.987 mL) was added dropwise at −70° C. under argon atmosphere. After stirring at −70° C. for 30 min, a solution of tert-butyl 4-formylpiperidine-1-carboxylate (526.64 mg, 2.47 mmol) in THF (1 mL) was added at −70° C. The resulting mixture was stirred at −70° C. for 3 h. Then the reaction was quenched with 1 M aq. NH₄C₁ (100 mL) and extracted with DCM (3×50 mL), dried over anhydrous sodium sulfate. The filtrate was concentrated under reduced pressure and the residue was purified by chromatography on silica gel eluted with 0%-30% EtOAc in PE to give tert-butyl 4-[hydroxy-[5-(2-isopropylphenyl)-1-(2-trimethylsilylethoxymethyl)pyrrolo[2,3-c]pyridin-3-yl]methyl]piperidine-1-carboxylate (500 mg, 862.30 umol, 35% yield) as a yellow solid. MS: m/z=580.35 [M+H]⁺.

### 5-(2-isopropylphenyl)-3-(4-piperidylmethyl)-1H-pyrrolo[2,3-c]pyridine

To a solution of tert-butyl 4-[hydroxy-[5-(2-isopropylphenyl)-1-(2-trimethylsilylethoxymethyl)pyrrolo[2,3-c]pyridin-3-yl]methyl]piperidine-1-carboxylate (500 mg, 862.30 μmol) and triethylsilane (1.00 g, 8.62 mmol, 1.38 mL) in dichloromethane (10 mL), boron trifluoride diethyl etherate (1.22 g, 8.62 mmol, 1.08 mL) was added at 0° C. under argon atmosphere. The resulting mixture was stirred at 20° C. for 16 h. The resulted reaction was concentrated under reduced pressure and the residue was purified by silica gel column chromatography, eluted with 5% NH₃·H₂O in MeOH to afford 5-(2-isopropylphenyl)-3-(4-piperidylmethyl)-1H-pyrrolo[2,3-c]pyridine (260 mg, 779.68 umol, 90% yield) as a light yellow semi-solid. MS: m/z=334.20 [M+H]⁺. ¹H NMR (400 MHz, Methanol-d₄): δ 9.04 (s, 1H), 8.18 (s, 1H), 8.10 (s, 1H), 7.63-7.59 (m, 2H), 7.54-7.43 (m, 2H), 3.41-3.33 (m, 2H), 3.01-2.93 (m, 4H), 2.83-2.75 (m, 1H), 2.22-1.94 (m, 3H), 1.77-1.56 (m, 4H), 1.24 (d, J=6.4 Hz, 6H).

### 5-(2-isopropylphenyl)-3-[[1-[1-methyl-4-(trifluoromethyl)imidazol-2-yl]-4-piperidyl]methyl]-1H-pyrrolo[2,3-c]pyridine

To a solution of 2-bromo-1-methyl-4-(trifluoromethyl)imidazole (82.41 mg, 359.85 umol) and 5-(2-isopropylphenyl)-3-(4-piperidylmethyl)-1H-pyrrolo[2,3-c]pyridine (100 mg, 299.88 μmol) in NMP (2 mL), cesium carbonate (293.12 mg, 899.63 umol) was added at room temperature. The resulting mixture was stirred for overnight at 140° C. The resulted mixture was diluted with water (50 mL), extracted with ethyl acetate (3×50 mL). The combined organic phase was washed with brine (60 mL), dried over anhydrous sodium sulfate. The filtrate was concentrated under reduced pressure and the residue was purified by Prep-TLC (MeOH:DCM=1:10) to give a residue (20 mg). The residue was continued purified by RP-Flash chromatography with the following conditions: Column: C18 gel column (40 g), 20-35 μm, 19×150 mm; mobile phase: acetonitrile and 5 mM aq. FA (Gradient: 10% hold 10 min, up to 30% within 20 min; Flow rate: 40 mL/min; Detector: UV 254 & 210 nm; RT: 18 min) to give 6 mg crude product (purity: 85%). The impure product was re-purified by Prep-HPLC with the following conditions: Column: XBridge Prep OBD C18 Column, 30×150 mm 5 um; Mobile Phase A: Water (10 mM aq. NH₄HCO₃+0.1% NH₃·H₂O), Mobile Phase B: ACN (0.1% DEA); Flow rate: 60 mL/min; Gradient: 50% B to 75% B in 8 min; Detector: UV 254 & 210 nm; RT: 7.23 min; to afford 5-(2-isopropylphenyl)-3-[[1-[1-methyl-4-(trifluoromethyl)imidazol-2-yl]-4-piperidyl]methyl]-1H-pyrrolo[2,3-c]pyridine (3 mg, 6.23 μmol, 2% yield) as a yellow semi-solid. MS: m/z=482.45 [M+H]⁺. ¹H NMR (400 MHz, Methanol-d₄): δ 8.69 (s, 1H), 8.11 (s, 1H), 7.56-7.38 (m, 3H), 7.31-7.24 (m, 3H), 3.54 (s, 3H), 3.30-3.24 (m, 2H), 3.15-3.02 (m, 1H), 2.84-2.75 (m, 3H), 1.84-1.81 (m, 2H), 1.53-1.45 (m, 2H), 1.35-1.31 (m, 2H), 1.18 (d, J=7.2 Hz, 6H)

### Example 37. Synthesis of 2-(2-isopropylphenyl)-7-[[4-[1-methyl-4-(trifluoromethyl)imidazol-2-yl]-1-piperidyl]methyl]-5H-pyrrolo[3,2-d]pyrimidine (Compound 6)

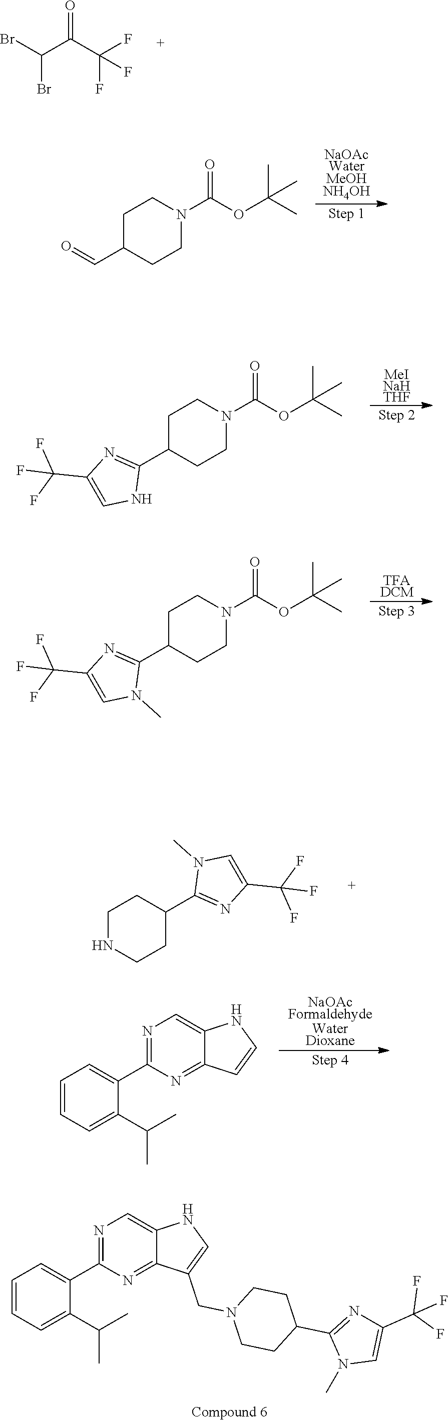

### Step 1: The synthesis of tert-butyl 4-[5-(trifluoromethyl)-1H-imidazol-2-yl]piperidine-1-carboxylate

To a solution of Sodium acetate (8.46 g, 103 mmol, 5.53 mL) in H₂O (50.0 mL) 3,3-dibromo-1,1,1-trifluoro-propan-2-one (13.9 g, 51.6 mmol, 7.03 mL) was added. The resulting mixture was stirred at 95° C. for 1 hr. The reaction mixture was cooled to room temperature and poured into a solution of tert-butyl 4-formylpiperidine-1-carboxylate (10.0 g, 46.8 mmol) in MeOH (225 mL) and 25% aqueous NH₃ (64.0 mL). The obtained mixture was stirred for 14 hr. at ambient temperature then concentrated in vacuo to give tert-butyl 4-[5-(trifluoromethyl)-1H-imidazol-2-yl]piperidine-1-carboxylate (12.5 g, 39.2 mmol, 83.49% yield) as a light-yellow solid which was used in the next step without further purification.

1H NMR (400 MHz, DMSO-d₆) δ 1.35 (s, 9H), 1.46-1.55 (m, 2H), 1.82-1.88 (m, 2H), 2.75-2.88 (m, 3H), 3.87-3.99 (m, 2H), 7.58 (s, 1H).

LCMS(ESI): [M−H]+ m/z: calcd 318.18; found 318.0.

### Step 2: The synthesis of tert-butyl 4-[1-methyl-4-(trifluoromethyl)imidazol-2-yl]piperidine-1-carboxylate

To a solution of tert-butyl 4-[4-(trifluormethyl)-1H-imidazol-2-yl]piperidine-1-carboxylate (2.00 g, 5.64 mmol) in THF (20.0 mL) sodium hydride (248 mg, 6.20 mmol, 60% dispersion in mineral oil) was added at 0° C. The reaction mixture was stirred at mom temperature for 1 hr. Methyl iodide (960 mg, 6.76 mmol, 421 μL) was added dropwise at 0° C. and the resulting mixture was stirred at mom temperature overnight. The reaction mixture was concentrated in vacuo. The residue was diluted with MTBE (30.0 mL) and water (20.0 mL). The organic layer was dried over anhydrous sodium sulfate, filtered and concentrated in vacuo to obtain tert-butyl 4-[1-methyl-4-(trifluoromethyl)imidazol-2-yl]piperidine-1-carboxylate (1.70 g, 5.10 mmol, 90.47% yield) as light-yellow oil which was used in the next step without further purification.

1H NMR (500 MHz, CDCl₃) δ 1.45 (s, 9H), 1.77-1.99 (m, 4H), 2.75-2.91 (m, 3H), 3.64 (s, 3H), 4.21 (br, 2H), 7.12 (s, 1H). LCMS(ESI): [M+H]+ m/z: calcd 334.2; found 334.2.

### Step 3: The synthesis of 4-[1-methyl-4-(trifluoromethyl)imidazol-2-yl]piperidine

To a stirred solution of tert-butyl 4-[1-methyl-4-(trifluoromethyl)imidazol-2-yl]piperidine-1-carboxylate (1.70 g, 4.59 mmol) in dry DCM (20.0 mL) TFA (2.62 g, 22.9 mmol, 1.77 mL) was added slowly. The resulting mixture was stirred at ambient temperature for 14 hours. The reaction mixture was concentrated in vacuo. The residue was dissolved in DCM (30 mL) and washed with 15%-water solution of potassium carbonate (20.0 mL). The organic layer was separated, dried over anhydrous sodium sulfate and concentrated in vacuo to afford 4-[1-methyl-4-(trifluoromethyl)imidazol-2-yl]piperidine (900 mg, 3.86 mmol, 84.07% yield) as brown oil which was used in the next steps without further purification. 1H NMR (400 MHz, CDCl₃) δ 1.84 (m, 4H), 2.67-2.87 (m, 3H), 3.22 (d, 2H), 3.62 (s, 3H), 7.09 (s, 1H).

LCMS(ESI): [M+H]+ m/z: calcd 234.14; found 234.2.

### Step 4: The synthesis of 2-(2-isopropylphenyl)-7-[[4-[1-methy-4-(trifluoromethyl)imidazol-2-yl]-1-piperidyl]methyl]-5H-pyrrolo[3,2-d]pyrimidine

To a solution of 2-(2-isopropylphenyl)-5H-pyrrolo[3,2-d]pyrimidine (114 mg, 416 μmol, HCl salt) in Dioxane (3.20 mL) and Water (2.00 mL) 4-[1-methyl-4-(trifluoromethyl)imidazol-2-yl]piperidine (97.12 mg, 416.4 μmol) was added, followed by Formaldehyde, 37% w/w aq. soln., stabilized with 7-8% methanol. (458 μmol, 35 μL). The resulting mixture was stirred at 60° C. for 16 hr. The reaction mixture was partially concentrated and diluted with 10% NaHCO₃ (10 mL) and EtOAc (15.0 mL). The organic layer was separated, dried over anhydrous sodium sulfate and concentrated in vacuo. The residue was subjected to prep HPLC (2-10 min 50-95% water -MeOH; flow: 30 mL/min, column: Waters SunFire C18, 100×19 mm, 5 μm) to give 56.8 mg of 80%-purity product (56.8 mg), which was re-purified by HPLC (0-5 min 55-90% water-methanol, +0.1% vol, of 25% aq. NH₃, 30 mL/min, column: YMC-Actus Triart C18, 100×20 mm, 5 μm) to afford 2-(2-isopropylphenyl)-7-[[4-[1-methyl-4-(trifluoromethyl)imidazol-2-yl]-1-piperidyl]methyl]-5H-pyrrolo[3,2-d]pyrimidine (36.5 mg, 75.6 μmol, 18.16% yield) as a light-yellow solid.

1H NMR (400 MHz, DMSO-d6) δ 1.17 (d, 6H), 1.64-1.78 (m, 4H), 2.09 (t, 2H), 2.60-2.68 (m, 1H), 2.95-3.00 (m, 2H), 3.54-3.60 (m, 4H), 3.78 (s, 2H), 7.25 (t, 1H), 7.38 (t, 1H), 7.44 (d, 1H), 7.55 (d, 1H), 7.62 (s, 1H), 7.85 (s, 1H), 8.96 (s, 1H), 11.77 (s, 1H). LCMS(ESI): [M+H]+ m/z: calcd 483.29; found 483.0.

### Example 38. 2-[4-[[5-(4-cyclopropyl-6-methoxy-pyrimidin-5-yl)-1H-pyrazolo[4,3-d]pyrimidin-3-yl]methyl]phenyl]-1-methyl-imidazole-4-carbonitrile (Compound 45)

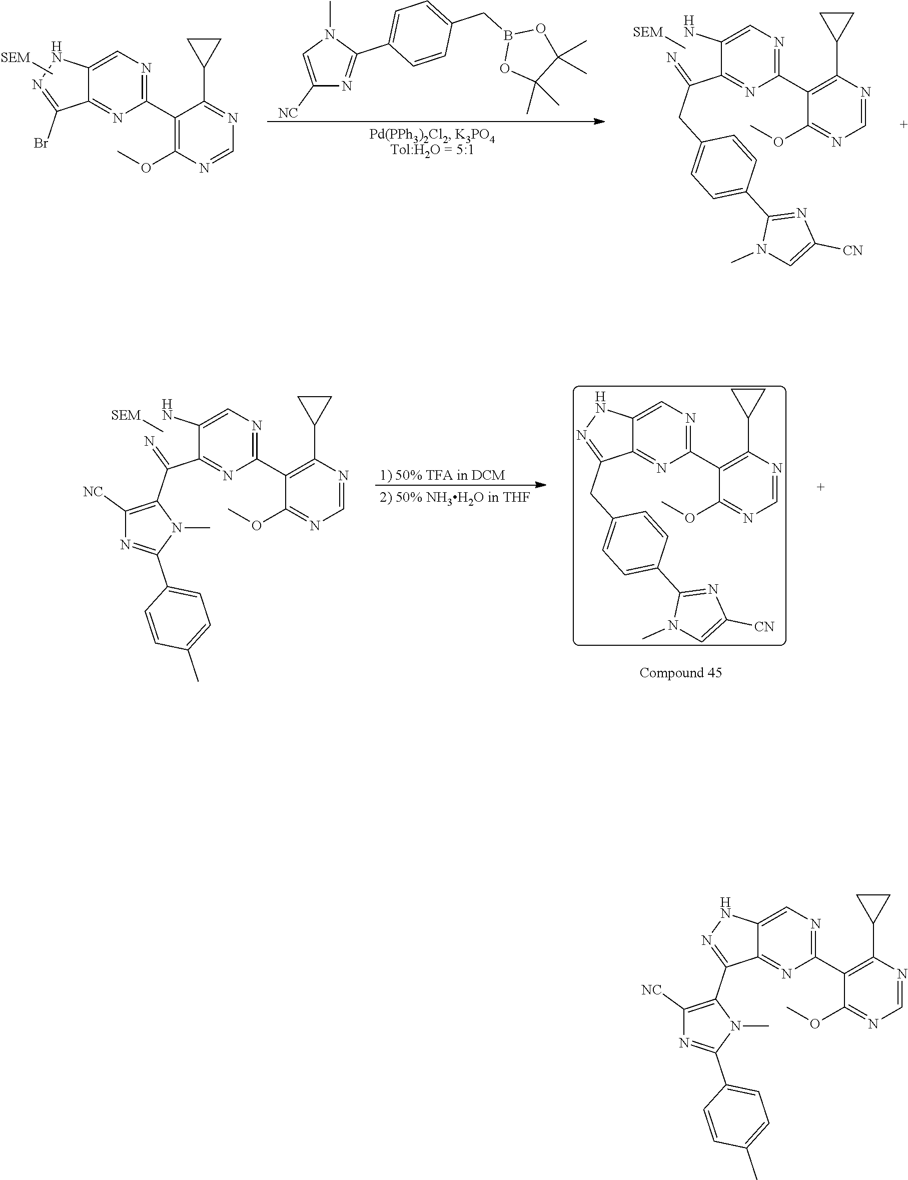

### 2-[4-[[5-(4-cyclopropyl-6-methoxy-pyrimidin-5-yl)-1-(2-trimethylsilylethoxymethyl)pyrazolo[4,3-d]pyrimidin-3-yl]methyl]phenyl]-1-methyl-imidazole-4-carbonitrile

To a solution of 2-[[3-bromo-5-(4-cyclopropyl-6-methoxy-pyrimidin-5-yl)pyrazolo[4,3-d]pyrimidin-1-yl]methoxy]ethyl-trimethyl-silane (200 mg, 576.09 μmol) and 1-methyl-2-[4-[(4,4,5,5-tetramethyl-1,3,2-dioxaborolan-2-yl)methyl]phenyl]imidazole-4-carbonitrile (139.64 mg, 576.09 μmol) in toluene:water=5:1 (1.8 mL) were added bis(triphenylphosphine)palladium(II) chloride (40.44 mg, 57.61 μmol) and anhydrous potassium phosphate (366.84 mg, 1728.27 μmol) at 25° C. under nitrogen atmosphere. Then the mixture was heated to 90° C., and stirred for 16 h under nitrogen atmosphere then cooled down to 25° C. The resulting mixture was diluted with ethyl acetate (100 mL) and washed with brine (3×50 mL), dried over anhydrous sodium sulfate. The organic phase was concentrated under reduced pressure. The residue was purified by silica gel column chromatography, eluted with 80%-90% ethyl acetate in petroleum ether to give 2-[4-[[5-(4-cyclopropyl-6-methoxy-pyrimidin-5-yl)-1-(2-trimethylsilylethoxymethyl)pyrazolo[4,3-d]pyrimidin-3-yl]methyl]phenyl]-1-methyl-imidazole-4-carbonitrile and 5-[5-(4-cyclopropyl-6-methoxy-pyrimidin-5-yl)-1-(2-trimethylsilylethoxymethyl)pyrazolo[4,3-d]pyrimidin-3-yl]-1-methyl-2-(p-tolyl)imidazole-4-carbonitrile as a mixture (80 mg, 172.61 μmol, 40% yield). The mixture was a yellow solid. MS: m/z=594.25 [M+H]⁺.

### 2-[4-[[5-(4-cyclopropyl-6-methoxy-pyrimidin-5-yl)-1H-pyrazolo[4,3-d]pyrimidin-3-yl]methyl]phenyl]-1-methyl-imidazole-4-carbonitrile

To a solution of 2-[4-[[5-(4-cyclopropyl-6-methoxy-pyrimidin-5-yl)-1-(2-trimethylsilylethoxymethyl)pyrazolo[4,3-d]pyrimidin-3-yl]methyl]phenyl]-1-methyl-imidazole-4-carbonitrile and 5-[5-(4-cyclopropyl-6-methoxy-pyrimidin-5-yl)-1-(2-trimethylsilylethoxymethyl)pyrazolo[4,3-d]pyrimidin-3-yl]-1-methyl-2-(p-tolyl)imidazole-4-carbonitrile (78 mg, 131.37 μmol, mixture, ratio in˜1:1) in DCM (2 mL) was added trifluoroacetic acid (2 mL) at 0° C. under nitrogen atmosphere. Then the reaction mixture was stirred at 25° C. for 2 h then concentrated under reduced pressure. The residue was added ammonium hydroxide (28% ammonia in water, 2 mL) and THF (2 mL) and stirred at 25° C. for 30 minutes. The reaction solution was concentrated under vacuum. The residue was purified by Prep-TLC (dichloromethane:methanol=20:1) to give 2-[4-[[5-(4-cyclopropyl-6-methoxy-pyrimidin-5-yl)-1H-pyrazolo[4,3-d]pyrimidin-3-yl]methyl]phenyl]-1-methyl-imidazole-4-carbonitrile as a crude product (A) and 5-[5-(4-cyclopropyl-6-methoxy-pyrimidin-5-yl)-1H-pyrazolo[4,3-d]pyrimidin-3-yl]-1-methyl-2-(p-tolyl)imidazole-4-carbonitrile as a crude product (B). Then, the crude product (A) was purified by RP-Flash with the following conditions: Column: C18 spherical Column, 20-35 μm, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 50% B in 10 min, 50% B to 50% B in 4 min, 50% B to 95% B in 10 min; Detector: UV 254 & 210 nm; RT: 19 min. The fractions of first eluting peak (RT: 18 min) were collected and concentrated under reduced pressure and then lyophilized overnight to afford 2-[4-[[5-(4-cyclopropyl-6-methoxy-pyrimidin-5-yl)-1H-pyrazolo[4,3-d]pyrimidin-3-yl]methyl]phenyl]-1-methyl-imidazole-4-carbonitrile (6.2 mg, 13.38 μmol, 10% yield) as an off-white solid. MS: m/z=464.15 [M+H]⁺. ¹H NMR (400 MHz, Methanol-d₄) δ 9.36 (s, 1H), 8.65 (s, 1H), 7.96 (s, 1H), 7.59 (s, 4H), 4.55 (s, 2H), 3.93 (s, 3H), 3.78 (s, 3H), 1.67-1.60 (m, 1H), 1.18-1.15 (m, 2H), 0.93-0.84 (m, 2H).

The crude product (B) was purified by RP-Flash with the following conditions: Column: C18 spherical Column, 20-35 μm, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 60% B in 10 min, 60% B to 60% B in 3 min, 60% B to 95% B in 8 min; Detector: UV 254 & 210 nm; RT: 18 min. The fractions of first eluting peak (RT: 18 min) were collected and concentrated under reduced pressure and then lyophilized overnight to afford 5-[5-(4-cyclopropyl-6-methoxy-pyrimidin-5-yl)-1H-pyrazolo[4,3-d]pyrimidin-3-yl]-1-methyl-2-(p-tolyl)imidazole-4-carbonitrile (4.7 mg, 10.14 μmol, 7% yield) as a white solid. MS: m/z=464.40. ¹H NMR (400 MHz, Methanol-d₄) δ 9.53 (s, 1H), 8.65 (s, 1H), 7.64 (d, J=8.0 Hz, 2H), 7.41 (d, J=8.0 Hz, 2H), 3.91 (s, 3H), 3.87 (s, 3H), 2.46 (s, 3H), 1.83-1.77 (m, 1H), 1.22-1.14 (m, 2H), 0.96-0.91 (m, 2H).

### Example 39. 2-(2-isopropylphenyl)-7-[[4-[1-methyl-4-(trifluoromethyl)imidazol-2-yl]piperazin-1-yl]methyl]-5H-pyrrolo[3,2-d]pyrimidine (Compound 35)

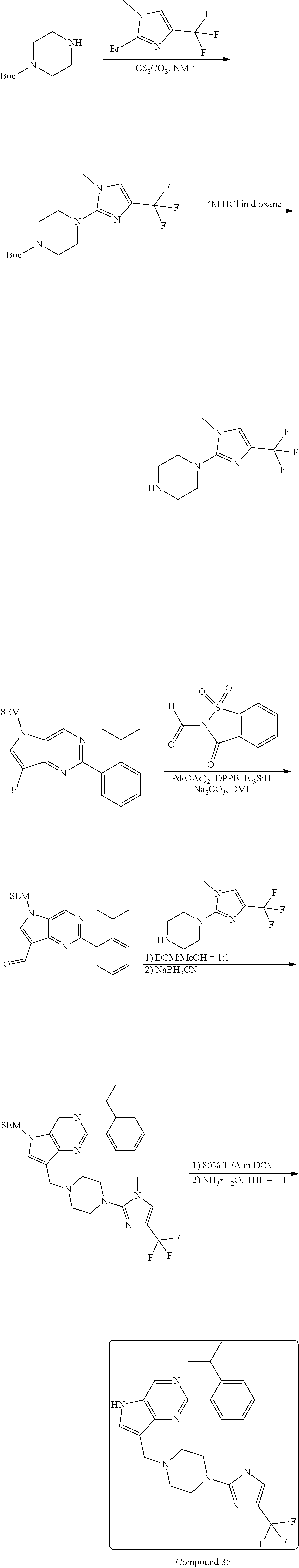

### tert-butyl 4-[1-methyl-4-(trifluoromethyl)imidazol-2-yl]piperazine-1-carboxylate

To a solution of tert-butyl piperazine-1-carboxylate (300 mg, 1.61 mmol) in 1-methyl-2-pyrrolidinone (3 mL) were added 2-bromo-1-methyl-4-(trifluormethyl)imidazole (368.85 mg, 1.61 mmol) and cesium carbonate (1.57 g, 4.83 mmol) at 25° C. under nitrogen atmosphere. After the mixture was heated to 140° C., and stirred for 10 h under nitrogen, it was cooled down to 25° C. Then the mixture was diluted with ethyl acetate (200 mL) and washed with brine (4×200 mL), dried over anhydrous sodium sulfate. The organic phase was concentrated under reduced pressure. The residue was purified by chromatography on silica gel elute with 45-55% ethyl acetate in petroleum ether to afford tert-butyl 4-[1-methyl-4-(trifluoromethyl)imidazol-2-yl]piperazine-1-carboxylate (200 mg, 598.20 μmol, 37% yield) as a yellow oil. MS: m/z=335.20 [M+H]⁺. ¹H NMR (400 MHz, Chloroform-d) δ 7.05 (s, 1H), 3.60-3.57 (m, 4H), 3.56 (s, 3H), 3.08 (t, J=5.2 Hz, 4H), 1.50 (s, 9H).

### 1-[1-methyl-4-(trifluoromethyl)imidazol-2-yl]piperazine

To a solution of 4 M hydrogen chloride in 1,4-dioxane (109.71 mmol, 5 mL) was added tert-butyl 4-[1-methyl-4-(trifluoromethyl)imidazol-2-yl]piperazine-1-carboxylate (200 mg, 598.20 μmol) at 25° C. under nitrogen. After the resulting mixture was stirred at 25° C. for 1 h, it was concentrated under reduced pressure. The residue was purified by silica gel chromatography, elute with 5%-10% methanol in dichloromethane to afford a crude product. The crude product was further purified by Prep-HPLC with the following conditions: Column: XBridge Prep OBD C18 Column, 30-150 mm 5 μm; Mobile Phase A: Water (10 mM aq. NH₄HCO₃), Mobile Phase B: MeCN; Flow rate: 60 mL/min; Gradient: 5% B to 27% B in 7 min, 27% B to 100% B in 10 min; Detector: UV 254 & 210 nm; RT: 5.92 min. The fractions of desired product were collected and concentrated under reduced pressure and then lyophilized overnight to give 1-[1-methyl-4-(trifluoromethyl)imidazol-2-yl]piperazine (50 mg, 213.47 μmol, 36% yield) as a yellow oil. MS: m/z=235.15 [M+H]⁺. ¹H NMR (400 MHz, Methanol-d₄) δ 7.35 (s, 1H), 3.59 (s, 3H), 3.10-3.02 (m, 4H), 3.02-2.94 (m, 4H).

### 2-(2-isopropylphenyl)-S-(2-trimethylsilylethoxymethyl)pyrrolo[3,2-d]pyrimidine-7-carbaldehyde

To a solution of 2-[[7-bromo-2-(2-isopropylphenyl)pyrrolo[3,2-d]pyrimidin-5-yl]methoxy]ethyl-trimethyl-silane (250 mg, 559.97 μmol) and 1,1,3-trioxo-1,2-benzothiazole-2-carbaldehyde (177.39 mg, 839.95 μmol) in N,N-dimethylformamide (2.5 mL) were added sodium carbonate (89.03 mg, 839.95 μmol, 35.19 μL), 4-diphenylphosphanylbutyl(diphenyl)phosphane (10.75 mg, 25.20 μmol), palladium acetate (3.77 mg, 16.80 μmol) and triethylsilane (130.22 mg, 1.12 mmol, 178.88 μL) at 25° C. under nitrogen atmosphere, and the reaction mixture was stirred at 25° C. for 10 minutes. Then the resulting mixture was heated to 80° C., and stirred for 16 h. The reaction mixture was cooled to 25° C. The reaction was diluted with ethyl acetate (150 mL), washed with brine (3×150 mL). The organic phase was dried over anhydrous sodium sulfate. After filtration, the filtrate was concentrated under reduced pressure. The reside was purified by Prep-TLC, eluted with 67% ethyl acetate in petroleum ether to give 2-(2-isopropylphenyl)-5-(2-trimethylsilylethoxymethyl)pyrrolo[3,2-d]pyrimidine-7-carbaldehyde (40 mg, 101.12 μmol, 18% yield) as a yellow oil. MS: m/z=396.25 [M+H]⁺.

### 2-[[2-(2-isopropylphenyl)-7-[[4-[1-methyl-4-(trifluoromethyl)imidazol-2-yl]piperazin-1-yl]methyl]pyrrolo[3,2-d]pyrimidin-5-yl]methoxy]ethyl-trimethyl-silane

To a solution of 2-(2-isopropylphenyl)-5-(2-trimethylsilylethoxymethyl)pyrrolo[3,2-d]pyrimidine-7-carbaldehyde (40 mg, 101.12 μmol) in methanol (0.5 mL) and dichloromethane (0.5 mL) was added 1-[1-methyl-4-(trifluoromethyl)imidazol-2-yl]piperazine (23.68 mg, 101.12 μmol) at room temperature under nitrogen atmosphere. The resulting mixture was heated to 40° C., and stirred for 4 h. Then the resulting mixture was cooled to 25° C., and to the above mixture was added sodium cyanoborohydride (6.35 mg, 101.12 μmol). The resulting mixture was stirred at 25° C. for 16 h. Then the mixture was diluted with ethyl acetate (100 mL), washed with brine (3×100 mL), dried over anhydrous sodium sulfate. The organic phase was concentrated under reduced pressure. The residue was purified by chromatography on silica gel elute with 45˜55% ethyl acetate in petroleum ether to afford 2-[[2-(2-isopropylphenyl)-7-[[4-[-methyl-4-(trifluoromethyl)imidazol-2-yl]piperazin-1-yl]methyl]pyrrolo[3,2-d]pyrimidin-5-yl]methoxy]ethyl-trimethyl-silane (30 mg, 48.88 μmol, 48% yield) as a yellow solid. MS: m/z=614.45 [M+H]⁺. ¹H NMR (400 MHz, Chloroform-d) crude H-NMR.

### 2-(2-isopropylphenyl)-7-[[4-[1-methyl-4-(trifluoromethyl)imidazol-2-yl]piperazin-1-yl]methyl]-5H-pyrrolo[3,2-d]pyrimidine

To a solution of 2-[[2-(2-isopropylphenyl)-7-[[4-[1-methyl-4-(trifluoromethyl)imidazol-2-yl]piperazin-1-yl]methyl]pyrrolo[3,2-d]pyrimidin-5-yl]methoxy]ethyl-trimethyl-silane (38 mg, 61.91 μmol) in dichloromethane (0.8 mL) was added trifluoroacetic acid (2.96 g, 25.96 mmol, 2 mL) at 0° C. under nitrogen atmosphere, and the reaction mixture was stirred at 25° C. for 2 h. The reaction solution was concentrated under reduced pressure. Then the mixture was added 28% aq. ammonium hydroxide (2 mL) and tetrahydrofuran (2 mL). The mixture was stirred at 25° C. for 30 minutes. The reaction solution was concentrated under reduced pressure. The residue was purified by chromatography on silica gel, eluted with 60˜70% ethyl acetate in petroleum ether to afford a crude product. Then, the crude product was further purified by RP-Flash chromatography with the following conditions: Column: C18 spherical Column, 20-35 um, 100A, 40 g; Mobile Phase A: Water (5 mM aq. NH₄HCO₃), Mobile Phase B: MeCN: Flow rate: 40 mL/min; Gradient: 5% B to 5% B in 5 min, 5% B to 55% B in 20 min, 55% B to 55% B in 4 min, 55% B to 95% B in 8 min; Detector: UV 254 & 210 nm; RT: 27 min. The fractions of product (RT: 27 min) were collected, concentrated under reduced pressure and then lyophilized overnight to afford 2-(2-isopropylphenyl)-7-[[4-[-methyl-4-(trifluoromethyl)imidazol-2-yl]piperazin-1-yl]methyl]-5H-pyrrolo[3,2-d]pyrimidine (7.5 mg, 15.51 μmol, 25% yield) as a white solid. MS: m/z=484.25 [M+H]⁺. ¹H NMR (400 MHz, Chloroform-d) δ 10.63 (br., 1H), 8.89 (s, 1H), 7.78 (s, 1H), 7.61-7.59 (m, 1H), 7.49-7.38 (m, 2H), 7.32-7.24 (m, 1H), 7.08 (s, 1H), 3.98 (s, 2H), 3.56 (s, 3H), 3.51-3.49 (m, 1H), 3.17 (t, J=4.8 Hz, 4H), 2.80 (t, J=4.8 Hz, 4H), 1.23 (d, J=6.8 Hz, 6H). ¹⁹F NMR (376 MHz, Chloroform-d) δ −62.722.

The Following Compounds were Prepared by Methods Similar to Those Exemplified Herein or Known to Persons of Skill in the Art

### Example 40. 2-[2-(azetidin-1-yl)-3-pyridyl]-7-[[4-[1-methyl-4-(trifluoromethyl)imidazol-2-yl]phenyl]methyl]-5H-pyrrolo[3,2-d]pyrimidine (Compound 67)

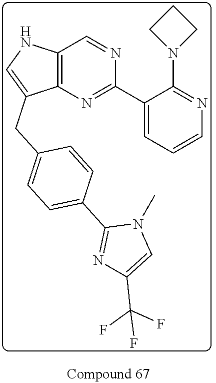 [SMILES: Cn1cc(C(F)(F)F)nc1-c1ccc(Cc2c[nH]c3cnc(-c4cccnc4N4CCC4)nc23)cc1]

### Prepared in a Manner Similar to Example 25

MS: m/z=490.15 [M+H]⁺. ¹H NMR (400 MHz, DMSO-d₆) δ 11.66 (s, 1H), 8.94 (s, 1H), 8.15 (dd, J=4.8, 1.6 Hz, 11H), 7.89 (s, 1H), 7.79 (s, 1H), 7.76-7.69 (m, 1H), 7.64 (d, J=8.0 Hz, 2H), 7.49 (d, J=8.0 Hz, 2H), 6.75 (dd, J=7.2, 4.8 Hz, 1H), 4.19 (s, 2H), 3.75 (s, 3H), 3.54 (t, J=7.6 Hz, 4H), 2.10-1.96 (m, 2H). ¹⁹F NMR (400 MHz, DMSO-d₆) δ −60.819

### Example 41, N,N-dimethyl-3-[7-[[4-[1-methyl-4-(trifluoromethyl)imidazol-2-yl]phenyl]methyl]-5H-pyrrolo[3,2-d]pyrimidin-2-yl]pyridin-2-amine (Compound 29)

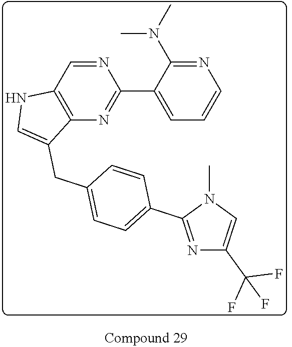 [SMILES: CN(C)c1ncccc1-c1ncc2[nH]cc(Cc3ccc(-c4nc(C(F)(F)F)cn4C)cc3)c2n1]

### Prepared in a Manner Similar to Example 25

MS: m/z=478.10 [M+H]⁺. ¹H NMR (400 MHz, DMSO-d₆) δ 11.65 (s, 1H), 8.94 (s, 1H), 8.14 (dd, J=4.8, 2.0 Hz, 1H), 7.90 (s, 1H), 7.79 (s, 1H), 7.75 (dd, J=7.2, 2.0 Hz, 1H), 7.66-7.58 (m, 2H), 7.52-7.48 (m, 2H), 6.78 (dd, J=7.6, 4.8 Hz, 1H), 4.16 (s, 2H), 3.75 (s, 3H), 2.63 (s, 6H). ¹⁹F NMR (376 MHz, DMSO-d₆) δ −60.804.

### Example 42. N-isopropyl-3-[7-[[4-[1-methyl-4-(trifluoromethyl)imidazol-2-yl]phenyl]methyl]-5H-pyrrolo[3,2-d]pyrimidin-2-yl]pyridin-2-amine (Compound 50)

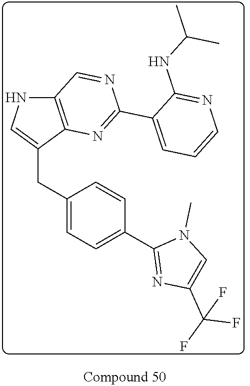 [SMILES: CC(C)Nc1ncccc1-c1ncc2[nH]cc(Cc3ccc(-c4nc(C(F)(F)F)cn4C)cc3)c2n1]

### Prepared in a Manner Similar to Example 25

MS: m/z=492.15 [M+H]⁺. ¹H NMR (400 MHz, DMSO-d₆) δ 11.74 (s, 1H), 9.87 (d, J=7.2 Hz, 1H), 9.04 (s, 1H), 8.76 (dd, J=7.6, 2.0 Hz, 1H), 8.13-8.12 (m, 1H), 7.89 (s, 1H), 7.84 (s, 1H), 7.63 (d, J=8.4 Hz, 1H), 7.46 (d, J=8.4 Hz, 1H), 6.66-6.62 (m, 11H), 4.35-4.28 (m, 1H), 4.26 (s, 2H), 3.73 (s, 3H), 1.20 (d.J=6.4 Hz, 6H). ¹⁹F NMR (400 MHz, DMSO-d₆) δ −60.815

### Example 43, tert-butyl 2-[2-(dimethylamino)pyridin-3-yl]-7-([4-[1-methyl-4-(trifluoromethyl)imidazol-2-yl]phenyl]methyl)pyrrolo[3,2-d]pyrimidine-5-carboxylate (Compound 44)

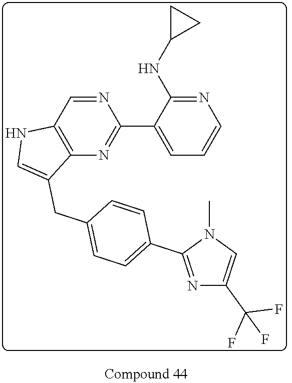 [SMILES: Cn1cc(C(F)(F)F)nc1-c1ccc(Cc2c[nH]c3cnc(-c4cccnc4NC4CC4)nc23)cc1]

### Prepared in a Manner Similar to Example 25

MS: m/z=490.10 [M+H]⁺. ¹H NMR (400 MHz, DMSO-d₆) δ 11.82 (s, 1H), 9.92 (d, J=3.6 Hz, 1H), 9.02 (s, 1H), 8.75 (dd, J=7.6, 2.0 Hz, 1H), 8.21-8.15 (m, 1H), 7.92-7.83 (m, 2H), 7.64 (d, J=8.4 Hz, 2H), 7.46 (d, J=8.0 Hz, 2H), 6.76-6.69 (m, 1H), 4.23 (s, 2H), 3.74 (s, 3H), 2.98-2.89 (m, 1H), 0.79-0.70 (m, 2H), 0.43-0.34 (m, 2H). ¹⁹F NMR (400 MHz, DMSO-d₆) δ −60.808

### Example 44. tert-butyl 7-[[4-[1-methyl-4-(trifluoromethyl)imidazol-2-yl]phenyl]methyl]-2-[2-(6-oxa-1-azaspiro[3.3]heptan-1-yl)-3-pyridyl]pyrrolo[3,2-d]pyrimidine-5-carboxylate (Compound 77)

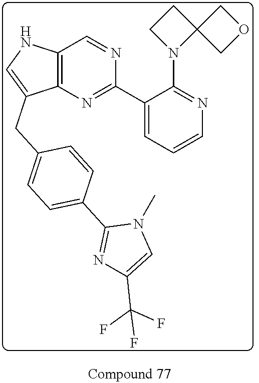 [SMILES: Cn1cc(C(F)(F)F)nc1-c1ccc(Cc2c[nH]c3cnc(-c4cccnc4N4CCC45COC5)nc23)cc1]

### Prepared in a Manner Similar to Example 25

MS: m/z=532.40 [M+H]⁺. ¹H NMR (400 MHz, Chloroform-d) δ 10.00 (s, 1H), 8.86 (s, 1H), 8.34 (dd, J=4.8, 1.6 Hz, 1H), 7.70 (dd, J=7.2, 1.6 Hz, 11H), 7.56 (d, J=8.4 Hz, 2H), 7.43 (d, J=8.4 Hz, 2H), 7.37 (s, 1H), 7.20 (s, 1H), 6.79-6.75 (m, 1H), 5.81 (d, J=6.4 Hz, 2H), 4.56 (d, J=6.4 Hz, 2H), 4.28 (s, 2H), 3.79 (s, 3H), 3.19 (t, J=7.2 Hz, 2H), 2.37 (t, J=7.2 Hz, 2H). ¹⁹F NMR (376 MHz, Chloroform-d) δ −62.505.

### Example 45. tert-butyl 7-[[4-[1-methyl-4-(trifluoromethyl)imidazol-2-yl]phenyl]methyl]-2-[2-(7-oxa-1-azaspiro[3.4]octan-1-yl)-3-pyridyl]pyrrolo[3,2-d]pyrimidine-5-carboxylate (Compound 32)

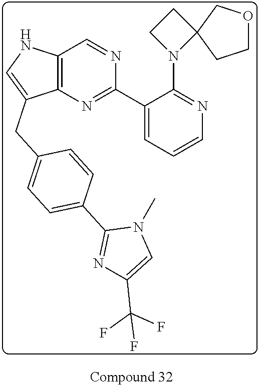 [SMILES: Cn1cc(C(F)(F)F)nc1-c1ccc(Cc2c[nH]c3cnc(-c4cccnc4N4CCC45CCOC5)nc23)cc1]

### Prepared in a Manner Similar to Example 25

MS: m/z=546.45 [M+H]⁺. ¹H NMR (400 MHz, Chloroform-d) δ 10.27 (s, 1H), 8.78 (s, 1H), 8.18-8.16 (m, 1H), 7.68-7.65 (m, 1H), 7.56 (d, J=8.0 Hz, 2H), 7.43 (d, J=8.4 Hz, 2H), 7.37 (s, 1H), 7.19 (d, J=2.4 Hz, 1H), 6.69 (dd, J=7.6, 4.8 Hz, 1H), 4.48 (d. J=8.8 Hz, 1H), 4.28-4.23 (m, 3H), 3.94-3.90 (m, 1H), 3.79 (s, 3H), 3.72 (d, J=8.8 Hz, 1H), 3.24-3.20 (m, 2H), 3.03-2.94 (m, 1H), 2.30-2.22 (m, 1H), 2.15-2.05 (m, 1H), 1.86-1.77 (m, 1H). ¹⁹F NMR (376 MHz, Chloroform-d) δ −62.476.

### Example 46. Synthesis of (2-(4-cyclopropyl-6-methoxypyrimidin-5-yl)-5H-pyrrolo[3,2-d]pyrimidin-7-yl)(4-(1-(1-methylazetidin-3-yl)-4-(trifluoromethyl)-1H-imidazol-2-yl)phenyl)methanol (Compound 7) 2 and -(4-cyclopropyl-6-methoxypyrimidin-5-yl)-7-(4-(1-(1-methylazetidin-3-yl)-4-(trifluoromethyl)-1H-imidazol-2-yl)benzyl)-5H-pyrrolo[3,2-d]pyrimidine (Compound 18)

### Prepared in a Manner Similar to Example 22

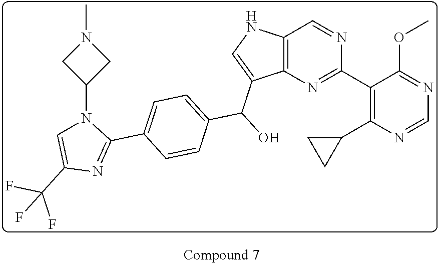 [SMILES: COc1ncnc(C2CC2)c1-c1ncc2[nH]cc(C(O)c3ccc(-c4nc(C(F)(F)F)cn4C4CN(C)C4)cc3)c2n1]

MS: m/z=577.20 [M+H]⁺. H NMR (400 MHz, Chloroform-d) δ 9.84 (s, 1H), 9.00 (s, 1H), 8.69 (s, 1H), 7.91 (s, 1H), 7.66 (d, J=8.0 Hz, 2H), 7.48 (d, J=8.0 Hz, 2H), 7.03 (s, 1H), 6.42 (s, 1H), 4.92-4.88 (m, 1H), 3.94 (s, 3H), 3.72 (t, J=8.0 Hz, 2H), 3.44 (t, J=8.0 Hz, 2H), 2.45 (s, 3H), 1.77-1.71 (m, 1H), 1.28-1.24 (m, 2H), 0.91-0.88 (m, 1H). ¹⁹F NMR (376 MHz, Chloroform-d) δ −62.564.

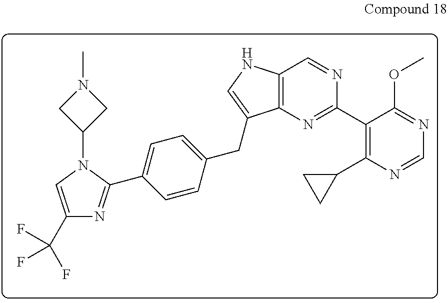 [SMILES: COc1ncnc(C2CC2)c1-c1ncc2[nH]cc(Cc3ccc(-c4nc(C(F)(F)F)cn4C4CN(C)C4)cc3)c2n1]

MS: m/z=561.25 [M+H]⁺; ¹H NMR (400 MHz, Chloroform-d) δ 9.96 (s, 1H), 8.97 (s, 1H), 8.67 (s, 1H), 7.90 (s, 1H), 7.46-7.38 (m, 4H), 7.12 (d, J=2.4 Hz, 1H), 4.90-4.84 (m, 1H), 4.28 (s, 2H), 3.93 (s, 3H), 3.72-3.66 (m, 2H), 3.42-3.38 (m, 2H), 2.42 (s, 3H), 1.74-1.71 (m, 1H), 1.28-1.21 (m, 2H), 0.90-0.82 (m, 2H); ¹⁹F NMR (376 MHz, Chloroform-d) δ −62.284, −62.509.

### Example 47. 2-[4-[[2-(4-cyclopropyl-6-methoxy-pyrimidin-5-yl)-5H-pyrrolo[3,2-d]pyrimidin-7-yl]-hydroxy-methyl]phenyl]-1-methyl-imidazole-4-carbonitrile (Compound 37) and 2-[4-[[2-(4-cyclopropyl-6-methoxy-pyrimidin-5-yl)-5H-pyrrolo[3,2-d]pyrimidin-7-yl]methyl]phenyl]-1-methyl-imidazole-4-carbonitrile (Compound 31)

### Prepared in a Manner Similar to Example 22

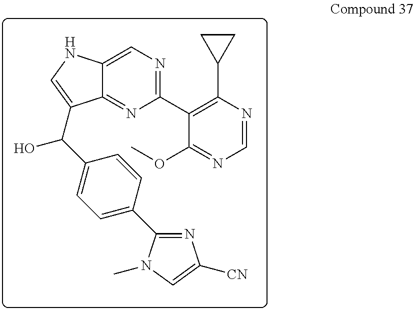 [SMILES: COc1ncnc(C2CC2)c1-c1ncc2[nH]cc(C(O)c3ccc(-c4nc(C#N)cn4C)cc3)c2n1]

MS: m/z=479.35 [M+H]⁺. ¹H NMR (400 MHz, Methanol-d₄) δ 8.98 (s, 1H), 8.63 (s, 1H), 7.96 (s, 1H), 7.79 (s, 1H), 7.73 (d, J=8.4 Hz, 2H) 7.64 (d, J=8.4 Hz, 2H), 6.42 (s, 1H), 3.92 (s, 3H), 3.80 (s, 3H), 1.68-1.57 (m, 1H), 1.20-1.09 (m, 2H), 0.94-0.83 (m, 2H).

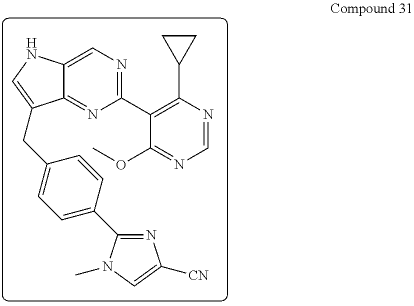 [SMILES: COc1ncnc(C2CC2)c1-c1ncc2[nH]cc(Cc3ccc(-c4nc(C#N)cn4C)cc3)c2n1]

MS: m/z=463.20 [M+H]⁺. ¹H NMR (400 MHz, Chloroform-d) δ 9.79 (s, 1H), 9.08 (s, 1H), 8.67 (s, 1H), 7.58-7.51 (m, 3H), 7.48 (d, J=8.0 Hz, 2H), 7.39 (s, 1H), 4.31 (s, 2H), 3.94 (s, 3H), 3.80 (s, 3H), 1.79-1.68 (m, 1H), 1.30-1.19 (m, 2H), 0.91-0.82 (m, 2H).

### Example 48. 2-(2-cyclopropylphenyl)-N-[4-[1-methyl-4-(trifluoromethyl)imidazol-2-yl]phenyl]-5H-pyrrolo[3,2-d]pyrimidin-7-amine Compound 12

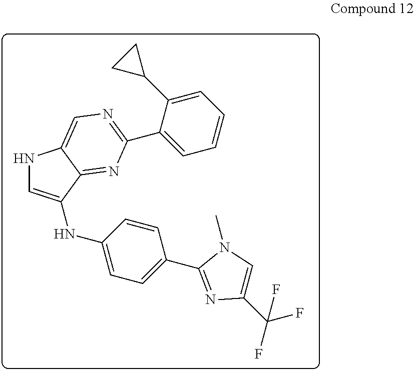 [SMILES: Cn1cc(C(F)(F)F)nc1-c1ccc(Nc2c[nH]c3cnc(-c4ccccc4C4CC4)nc23)cc1]

### Prepared in a Manner Similar to Example 11

H NMR (400 MHz, Chloroform-d) δ 9.08 (s, 1H), 7.65 (d, J=7.2 Hz, 1H), 7.49 (d, J=8.4 Hz, 2H), 7.40-7.30 (m, 3H), 7.30-7.27 (m, 1H), 7.08-6.98 (m, 3H), 6.36 (br., 1H), 3.78 (s, 3H), 2.43-2.39 (s, 1H), 0.77-0.73 (m, 2H), 0.65-0.62 (m, 2H), ¹⁹F NMR (376 MHz, Chloroform-d) δ −62.27.

### Example 49. 2-(2-cyclopropylphenyl)-N-methyl-N-[4-[1-methyl-4-(trifluoromethyl)imidazol-2-yl]phenyl]-5H-pyrrolo[3,2-d]pyrimidin-7-amine (Compound 13)

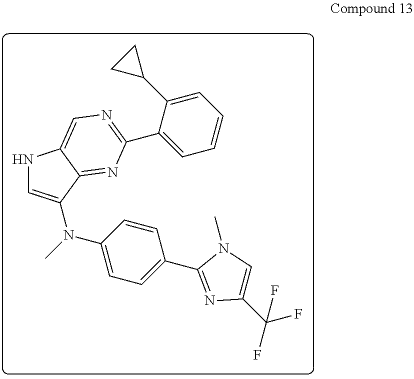 [SMILES: CN(c1ccc(-c2nc(C(F)(F)F)cn2C)cc1)c1c[nH]c2cnc(-c3ccccc3C3CC3)nc12]

### Prepared by Methylation of a Protected Precursor of Compound 12

MS: m/z=489.15 [M+H]⁺. ¹H NMR (400 MHz, Chloroform-d) δ 11.85 (br., 1H), 9.07 (s, 1H), 7.68 (d, J=6.0 Hz, 1H), 7.41-7.32 (m, 3H), 7.23 (t, J=7.2 Hz, 1H), 7.05 (s, 1H), 7.00 (d, J=7.6 Hz, 1H), 6.81 (d, J=8.8 Hz, 2H), 3.76 (s, 3H), 3.49 (s, 3H), 0.67 (d, J=8.0 Hz, 2H), 0.57 (t, J=5.2 Hz, 2H). ¹⁹F NMR (376 MHz, Chloroform-d) δ −62.19

### Example 50. 2-(2-cyclopropyl-3-pyridyl)-N-[4-[1-methyl-4-(trifluoromethyl)imidazol-2-yl]phenyl]-5H-pyrrolo[3,2-d]pyrimidin-7-amine (Compound 43)

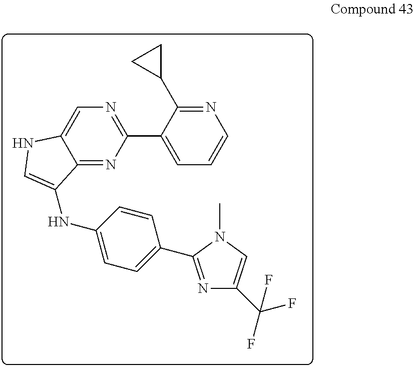 [SMILES: Cn1cc(C(F)(F)F)nc1-c1ccc(Nc2c[nH]c3cnc(-c4cccnc4C4CC4)nc23)cc1]

### Prepared in a Manner Similar to Example 11

MS: m/z=475.17 [M+H]⁺. ¹H NMR (400 MHz, Chloroform-d) δ 11.43 (br., 1H), 9.07 (s, 1H), 8.55 (s, 1H), 8.13 (s, 1H), 7.46 (d, J=8.4 Hz, 2H), 7.40 (s, 1H), 7.27-7.16 (m, 1H), 6.96 (d, J=8.4 Hz, 2H), 6.10 (br., 1H), 3.79 (s, 3H), 2.75-2.72 (s, 1H), 1.28-1.25 (m, 2H), 0.89-0.80 (m, 2H); ¹⁹F NMR (400 MHz, Chloroform-d) δ −62.16.

### Example 51. 5-(2-isopropylphenyl)-N-[4-[1-methyl-4-(trifluoromethyl)imidazol-2-yl]phenyl]-1H-pyrrolo 2,3-c]pyridin-3-amine (Compound 17)

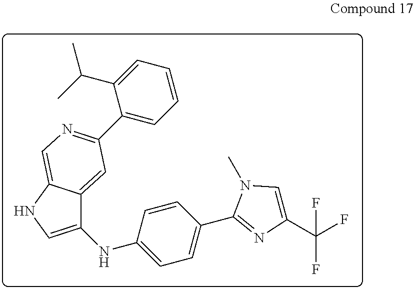 [SMILES: CC(C)c1ccccc1-c1cc2c(Nc3ccc(-c4nc(C(F)(F)F)cn4C)cc3)c[nH]c2cn1]

### Prepared in a Manner Similar to Example 11

MS: m/z=476.20 [M+H]⁺. ¹H NMR (400 MHz, Chloroform-d) δ 10.38 (br., 1H), 8.95 (s, 11H), 7.54-7.34 (m, 5H), 7.32-7.28 (m, 2H), 7.24-7.20 (m, 1H), 7.05 (s, 1H), 6.76 (d, J=8.4 Hz, 2H), 5.55 (s, 1H), 3.75 (s, 3H), 3.22-3.15 (m, 1H), 1.13 (d, J=6.8 Hz, 6H); ¹⁹F NMR (376 MHz, Chloroform-d) δ −62.48.

### Example 52. S-(4-cyclopropyl-6-methoxypyrimidin-5-yl)-3-(4-(1-ethyl-4-(trifluoromethyl)-1H-imidazol-2-yl)benzyl)-1H-pyrazolo[4,3-d]pyrimidine Compound 165

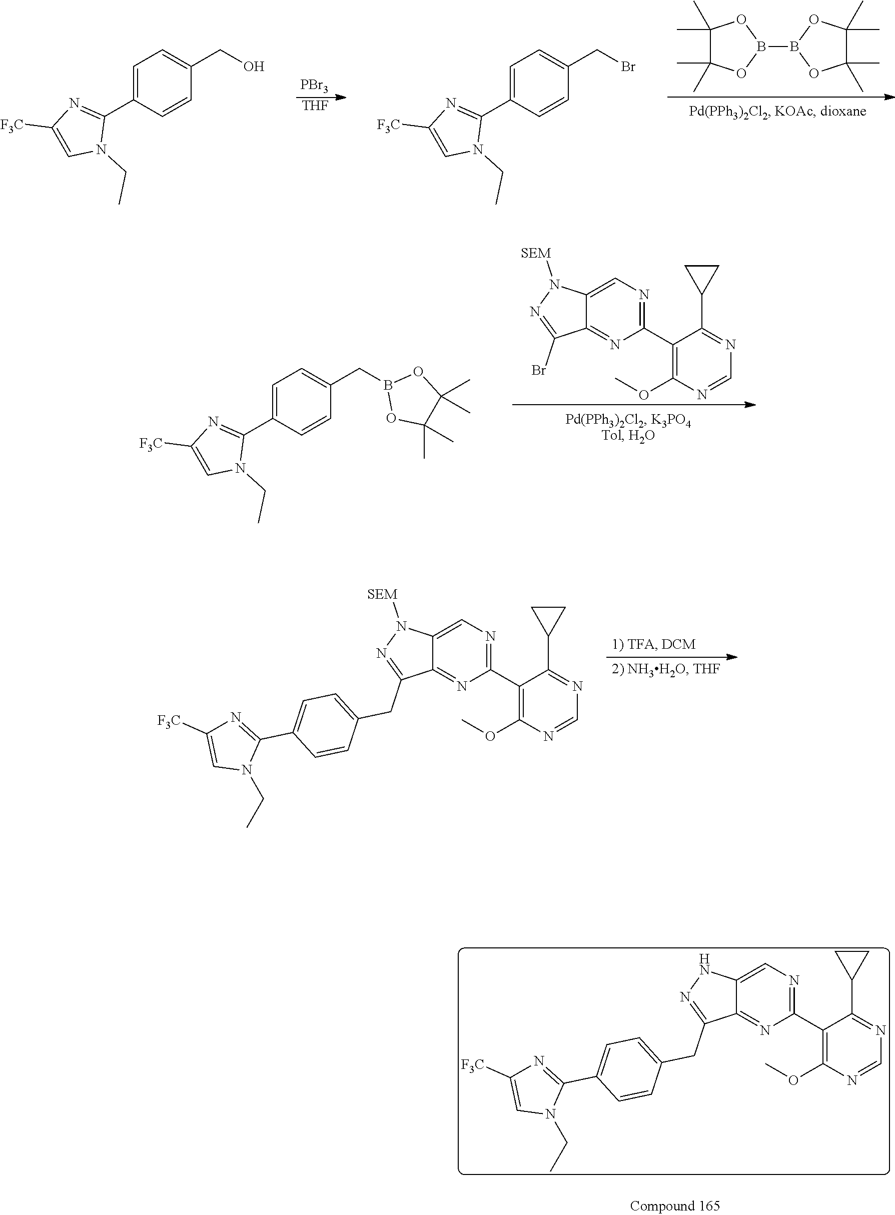

### 1.1 2-(4-(bromomethyl)phenyl)-1-ethyl-4-(trifluoromethyl)-1H-imidazole

To a solution of [4-[1-ethyl-4-(trifluoromethyl)imidazol-2-yl]phenyl]methanol (740 mg, 2.74 mmol) in THF (10 mL) was added phosphorus tribromide (3.71 g, 13.69 mmol, 1.29 mL) at 25° C., the resulting solution was stirred for 2 hours at 25° C. The resulted solution was quenched by saturated sodium bicarbonate aqueous solution (100 mL), extracted by ethyl acetate (2×50 mL). The combined organic layers were washed with brine (2×30 mL), dried over anhydrous sodium sulfate. After filtration, the filtrate was concentrated under reduced pressure. The residue was purified by silica gel column chromatography, eluted with 40%-60% EtOAc in PE to afford 2-[4-(bromomethyl)phenyl]-1-ethyl-4-(trifluoromethyl)imidazole (890 mg, 2.67 mmol, 98% yield) as a yellow solid. MS: m/z=332.95, 334.95 [M+H]⁺; ¹H NMR (400 MHz, Chloroform-d) δ 7.61-7.58 (m, 2H), 7.53-7.51 (m, 2H), 7.40 (s, 1H), 4.55 (s, 2H), 4.16-4.08 (m, 2H), 1.47 (t, J=7.6 Hz, 3H); ¹⁹F NMR (376 MHz, Chloroform-d) δ −62.69.

### 1.2 1-ethyl-2-(4-((4,4,5,5-tetramethyl-1,3,2-dioxaborolan-2-yl)methyl)phenyl)-4-(trifluoromethyl)-1H-imidazole

To a solution of 2-[4-(bromomethyl)phenyl]-1-ethyl-4-(trifluoromethyl)imidazole (840 mg, 2.52 mmol) and 4,4,5,5-tetramethyl-2-(4,4,5,5-tetramethyl-1,3,2-dioxaborolan-2-yl)-1,3,2-dioxaborolane (960 mg, 3.8 mmol) in 1,4-dioxane (10 mL) were added potassium acetate (742 mg, 7.6 mmol) and bis(triphenylphosphine)palladium(II) chloride (177 mg, 252 μmol) at 25° C. The resulting mixture was stirred for 1 hour at 80° C. then naturally cooled down to 25° C. The mixture was filtered through a Celite pad, the filtrate was concentrated under reduced pressure. The residue was purified by silica gel column chromatography, eluted with 30%-50% EtOAc in PE to afford 1-ethyl-2-(4-((4,4,5,5-tetramethyl-1,3,2-dioxaborolan-2-yl)methyl)phenyl)-4-(trifluoromethyl)-1H-imidazole (400 mg, 1.05 mmol, 42% yield) as an off-white solid. MS: m/z =381.30 [M+H]⁺. ¹H NMR (400 MHz, Chloroform-d) δ 7.49-7.45 (m, 2H), 7.36 (s, 1H), 7.31-7.29 (m, 2H), 4.12-4.05 (m, 2H), 2.36 (s, 2H), 1.45 (t, J=7.2 Hz, 3H), 1.25 (s, 12H). ¹⁹F NMR (376 MHz, Chloroform-d) δ −62.61.

### 1.3 5-(4-cyclopropyl-6-methoxypyrimidin-5-yl)-3-(4-(1-ethyl-4-trifluoromethyl)-1H-imidazol-2-yl)benzyl)-1-((2-(trimethylsilyl)-ethoxy)methyl)-1H-pyrazolo[4,3-d]pyrimidine

To a solution of 1-ethyl-2-(4-((4,4,5,5-tetramethyl-1,3,2-dioxaborolan-2-yl)methyl)phenyl)-4-(trifluoromethyl)-1H-imidazole (400 mg, 1.05 mmol) and 3-bromo-5-(4-cyclopropyl-6-methoxypyrimidin-5-yl)-1-((2-(trimethylsilyl)ethoxy)methyl)-1H-pyrazolo[4,3-d]pyrimidine (502 mg, 1.05 mmol) in toluene (5 mL) and H₂O (1 mL) were added dichloropalladium triphenyl phosphane (74 mg, 105 μmol) and potassium phosphate (447 mg, 2.10 mmol) at 25° C. After the resulting mixture was stirred for 16 hours at 90° C., it was diluted by ethyl acetate (30 mL), then was filtered through a Celite pad. The filtrate was concentrated under reduced pressure. The residue was purified by silica gel column chromatography, eluted with 10%-30% EtOAc in PE to afford 5-(4-cyclopropyl-6-methoxypyrimidin-5-yl)-3-(4-(1-ethyl-4-(trifluoromethyl)-1H-imidazol-2-yl)benzyl)-1-((2-(trimethylsilyl)ethoxy)methyl)-1H-pyrazolo[4,3-d]pyrimidine (150 mg, crude product). MS: m/z=651.35 [M+H]⁺.

### 1.4 5-(4-cyclopropyl-6-methoxypyrimidin-5-yl)-3-(4-(1-ethyl-4-(trifluoromethyl)-1H-imidazol-2-yl)benzyl)-1H-pyrazolo[4,3-d]pyrimidine

The solution of 5-(4-cyclopropyl-6-methoxypyrimidin-5-yl)-3-(4-(1-ethyl-4-(trifluoromethyl)-1H-imidazol-2-yl)benzyl)-1-((2-(trimethylsilyl)ethoxy)methyl)-1H-pyrazolo[4,3-d]pyrimidine (150 mg, 230.50 μmol) in DCM (2 mL) and trifluoroacetic acid (2 mL) was stirred for 3 hours at 25° C., and the resulting solution was concentrated under reduced pressure. Tetrahydrofuran (2 mL) and ammonium hydroxide (2 mL, 28% NH₃H₂O in H₂O) were added to the above residue, then the resulting solution was stirred for 10 minutes. The solution was concentrated under reduced pressure. The residue was purified by Prep-TLC (PE: EA=1:1) to afford crude product. The crude product was further purified by RP-Flash chromatography with the following conditions: Column: C18 spherical, 20-35 um, 100A, 80 g; Mobile Phase A: 10 mM aq. NH₄HCO₃, Mobile Phase B: MeCN; Flow rate: 70 mL/min; Gradient: 5% B to 5% B in 10 min, 5% B to 25% B in 25 min; 25% B to 25% B in 5 min; 25% B to 95% B in 5 min; Detector: UV 254 & 220 nm; RT: 37 min to give 5-(4-cyclopropyl-6-methoxy-pyrimidin-5-yl)-3-[[4-[l-ethyl-4-(trifluoromethyl)imidazol-2-yl]phenyl]methyl]-1H-pyrazolo[4,3-d]pyrimidine (65 mg, 124.88 μmol, 54% yield) as an off-white solid. MS: m/z=521.40 [M+H]⁺. ¹H NMR (400 MHz, Chloroform-d) 13.64 (s, 1H), 9.17 (s, 1H), 8.68 (s, 1H), 7.65-7.63 (m, 2H), 7.60-7.58 (m, 2H), 7.47 (s, 1H), 4.54 (s, 2H), 4.17-4.12 (m, 2H), 3.91 (s, 3H), 1.68-1.63 (m, 1H), 1.50 (t, J=7.2 Hz, 3H), 1.24-1.20 (m, 2H), 0.85-0.81 (m, 2H). ¹⁹F NMR (376 MHz, Chloroform-d) δ −62.11.

### Example 53. 5-(4-cyclopropyl-6-methoxypyrimidin-5-yl)-3-(4-(1-ethyl-4-(trifluoromethyl-1H-imidazol-2-yl)benzyl)-1-methyl-1H-pyrazolo[4,3-d]pyrimidine (Compound 106) & 5-(4-cyclopropyl-6-methoxypyrimidin-5-yl)-3-(4-(1-ethyl-4-(trifluoromethyl)-1H-imidazol-2-yl)benzyl)-2-methyl-2H-pyrazolo[4,3-d]pyrimidine (Compound 139)

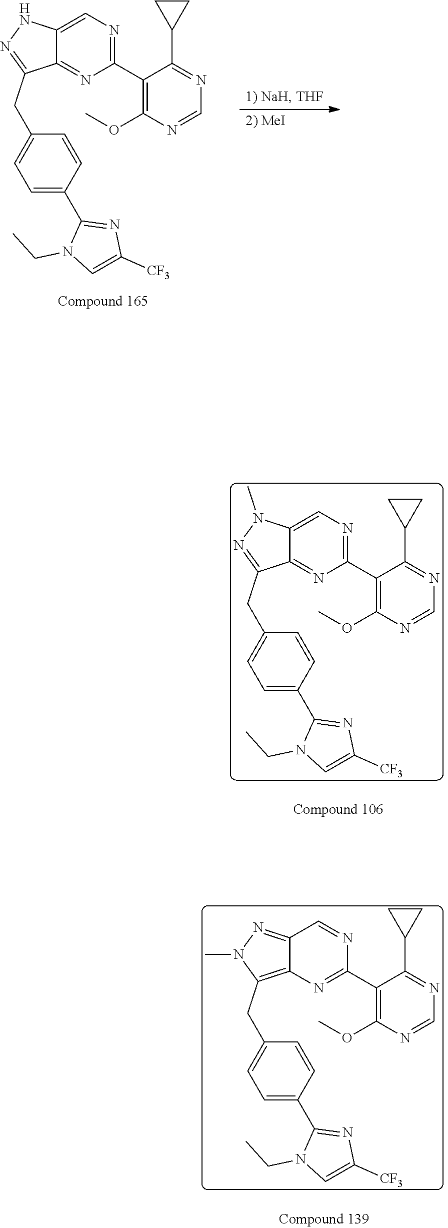

### 1.1 5-(4-cyclopropyl-6-methoxypyrimidin-5-yl)-3-(4-(1-ethyl-4-(trifluoromethyl)-1H-imidazol-2-yl)benzyl)-1-methyl-1H-pyrazolo[4,3-d]pyrimidine (Compound 106) & 5-(4-cyclopropyl-6-methoxypyrimidin-5-yl)-3-(4-(1-ethyl-4-(trifluoromethyl)-1H-imidazol-2-yl)benzyl)-2-methyl-2H-pyrazolo[4,3-d]pyrimidine (Compound 139)

To a stirred solution of 5-(4-cyclopropyl-6-methoxy-pyrimidin-5-yl)-3-[[4-[1-ethyl-4-(trifluoromethyl)imidazol-2-yl]phenyl]methyl]-1H-pyrazolo[4,3-d]pyrimidine (30 mg, 57.64 μmol) in THF (2 mL) was added sodium hydride (3 mg, 74 μmol, 60% in mineral oil) at 0° C. The resulting mixture was stirred for 1 hour at 25° C., then Iodomethane (5.5 mg, 38 μmol, 2.4 μL) was added to the above mixture at 25° C. The resulting mixture was stirred for 2 hours at 25° C. then quenched by saturated ammonium chloride aqueous solution (2×10 mL), extracted by ethyl acetate (2×15 mL). The combined organic layers were washed with brine (2×10 mL), dried over anhydrous sodium sulfate. After filtration, the filtrate was concentrated under reduced pressure. The residue was purified by Prep-TLC (dichloromethane:methanol=15:1) to afford the crude mixture of two isomers. The crude mixture was further purified by Prep-HPLC with the following conditions: Column: XSelect CSH Prep C18 OBD Column, 19×250 mm, 5 μm; Mobile Phase A: 5 mM aq. FA, Mobile Phase B: ACN; Flow rate: 25 mL/min; Gradient: 40% B to 50% B in 12 min; Detector: UV 254 & 210 nm; RT: 11.0 min to give 5-(4-cyclopropyl-6-methoxypyrimidin-5-yl)-3-(4-(1-ethyl-4-(trifluoromethyl)-1H-imidazol-2-yl)benzyl)-1-methyl-1H-pyrazolo[4,3-d]pyrimidine (4.2 mg, 7.86 μmol, 20% yield, Compound 106) as an off-white solid. MS: m/z=535.30 [M+H]⁺; ¹H NMR (400 MHz, Chloroform-d) δ 9.23 (s, 1H), 8.70 (s, 1H), 7.58-7.55 (m, 2H), 7.51-7.48 (m, 2H), 7.36 (s, 1H), 4.52 (s, 2H), 4.22 (s, 3H), 4.08-4.02 (m, 2H), 3.94 (s, 3H), 1.70-1.63 (m, 1H), 1.43 (t, J=7.2 Hz, 3H), 1.28-1.23 (m, 2H), 0.90-0.86 (m, 2H); ¹⁹F NMR (376 MHz, Chloroform-d) δ −62.70.

The second product containing (RT2: 10.5 min) peak was combined and concentrated under reduced pressure to give 5-(4-cyclopropyl-6-methoxypyrimidin-5-yl)-3-(4-(1-ethyl-4-(trifluoromethyl)-1H-imidazol-2-yl)benzyl)-2-methyl-2H-pyrazolo[4,3-d]pyrimidine (7 mg, 13.1 μmol, 34% yield, Compound 139) as an off-white solid. MS: m/z=535.30 [M+H]⁺; ¹H NMR (400 MHz, Chloroform-d) δ 9.54 (s, 11H), 8.69 (s, 1H), 7.55-7.52 (m, 2H), 7.38 (s, 1H), 7.35-7.32 (m, 2H), 4.64 (s, 21H), 4.15 (s, 3H), 4.08-4.02 (m, 2H), 3.97 (s, 3H), 1.80-1.73 (m, 1H), 1.43 (t, J=7.2 Hz, 3H), 1.29-1.25 (m, 2H), 0.95-0.91 (m, 2H); ¹⁹F NMR (376 MHz, Chloroform-d) δ −62.73.

### Example 54. 2-(4-cyclopropyl-6-methoxy-pyrimidin-5-yl)-7-[[4-[1-(oxetan-3-yl)-4-(trifluoromethyl)imidazol-2-yl]phenyl]methyl]-5H-pyrrolo[3,2-d]pyrimidine Compound 154

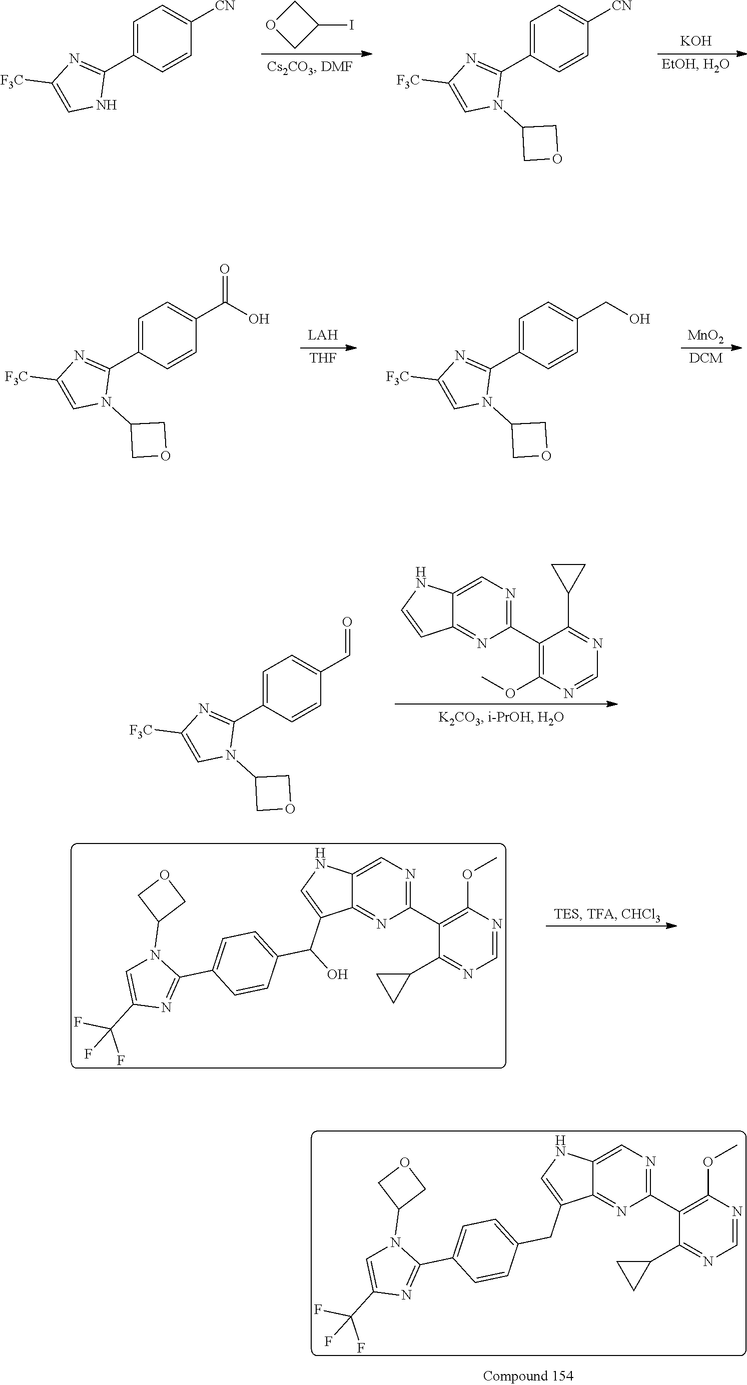

### 1.1 4-[1-(oxetan-3-yl)-4-(trifluoromethyl)imidazol-2-yl]benzonitrile

To a stirred solution of 4-[4-(trifluoromethyl)-1H-imidazol-2-yl]benzonitrile (1 g, 4.22 mmol) and 3-iodooxetane (2.33 g, 12.65 mmol, 1.09 mL) in N,N-dimethylformamide (15 mL) was added cesium carbonate (5.49 g, 16.86 mmol) at 25° C. under nitrogen atmosphere. The resulting mixture was stirred at 110° C. for 18 hours under nitrogen atmosphere then diluted with ethyl acetate (100 mL). The resulting mixture was washed with brine (50 mL), dried over anhydrous sodium sulfate. After filtration, the filtrate was concentrated under reduced pressure. The residue was purified by Prep-HPLC with the following conditions: Column: Xselect CSH OBD Column 30×150 mm, 5 μm; Mobile Phase A: ACN Mobile Phase B: Water (5 mM aq. TFA); Flow rate: 60 mL/min; Gradient: 15% B to 35% B in 8 min, 35% B to 35% B in 15 min; Detector: UV 254 & 210 nm; RT: 11.7 min. The fractions were collected, concentrated under reduced pressure and then lyophilized overnight to afford 4-[1-(oxetan-3-yl)-4-(trifluoromethyl)imidazol-2-yl]benzonitrile (300 mg, 1.02 mmol, 24% yield) as a yellow solid. MS: m/z=293.95 [M+H]⁺. ¹H NMR (400 MHz, Chloroform-d) δ 7.96 (s, 1H), 7.86-7.78 (m, 2H), 7.65-7.58 (m, 2H), 5.49-5.43 (m, 1H), 5.17-5.09 (m, 2H), 4.91-4.83 (m, 2H).

### 1.2 4-[1-(oxetan-3-yl)-4-(trifluoromethyl)imidazol-2-yl]benzoic Acid

To a stirred solution of 4-[1-(oxetan-3-yl)-4-(trifluoromethyl)imidazol-2-yl]benzonitrile (300 mg, 1.02 mmol) in ethanol (2.5 mL) and water (2.5 mL) was added potassium hydroxide (287 mg, 5.1 mmol) at 25° C. under nitrogen atmosphere. The resulting mixture was stirred at 90° C. for 3 hours under nitrogen atmosphere. The mixture was acidified pH to 5 with 1 mol/L hydrogen chloride aqueous solution. The precipitate was filtered, the filter cake was washed with cool water (3×10 mL), dried oven to give 4-[1-(oxetan-3-yl)-4-(trifluoromethyl)imidazol-2-yl]benzoic acid (200 mg, 640.5 μmol, 62% yield) as a white solid. MS: m/z=313.00 [M+H]⁺. ¹H NMR (400 MHz, DMSO-d₆) δ 13.21 (s, 1H), 8.54 (s, 1H), 8.10-8.03 (m, 2H), 7.72-7.65 (m, 2H), 5.63-5.56 (m, 1H), 4.92-4.89 (m, 2H), 4.83-4.80 (m, 2H).

### 1.3 [4-[1-(oxetan-3-yl)-4-(trifluoromethyl)imidazol-2-yl]phenyl]methanol

To a stirred solution of 4-[1-(oxetan-3-yl)-4-(trifluoromethyl)imidazol-2-yl]benzoic acid (200 mg, 640.5 μmol) in THF (5 mL) was added lithium aluminum hydride (73 mg, 1.9 mmol) at 0° C. under nitrogen atmosphere. The resulting mixture was stirred at this temperature for 1 hour, then stirred at 25° C. for 18 hours under nitrogen atmosphere. The reaction was quenched by the addition of saturated ammonium chloride aqueous (10 mL) at 0° C. then extracted with ethyl acetate (3×50 mL). The combined organic layers were washed with brine (50 mL), dried over anhydrous sodium sulfate. After filtration, the filtrate was concentrated under reduced pressure. The residue was purified by silica gel column chromatography, eluted with 1%-30% ethyl acetate in petroleum ether to give [4-[1-(oxetan-3-yl)-4-(trifluoromethyl)imidazol-2-yl]phenyl]methanol (150 mg, 503 μmol, 78% yield) as a yellow solid. MS: m/z=299.00 [M+H]⁺. ¹H NMR (400 MHz, Chloroform-d) δ 7.90 (s, 1H), 7.51 (d, J=8.0 Hz, 2H), 7.45 (d, J=8.0 Hz, 2H), 5.52-5.44 (m, 1H), 5.11-5.06 (m, 2H), 4.89-4.84 (m, 2H), 4.80 (d, J=5.2 Hz, 2H).

### 1.4 4-[1-(oxetan-3-yl)-4-(trifluoromethyl)imidazol-2-yl]benzaldehyde

To a stirred solution of [4-[1-(oxetan-3-yl)-4-(trifluoromethyl)imidazol-2-yl]phenyl]methanol (130 mg, 436 μmol) in DCM (2 mL) was added manganese dioxide (3789 mg, 4.4 mmol) at 25° C. under nitrogen atmosphere. After the resulting mixture was stirred at 40° C. for 16 hours under nitrogen atmosphere, it was filtered and the filter cake was washed with ethyl acetate (3×10 mL). The filtrate was concentrated under reduced pressure. The residue was purified by silica gel column chromatography, eluted with 1%-50% ethyl acetate in petroleum ether to give 4-[1-(oxetan-3-yl)-4-(trifluoromethyl)imidazol-2-yl]benzaldehyde (105 mg, 354.44 μmol, 80% yield) as a light-yellow solid. MS: m/z=297.10 [M+H]⁺.

### 1.5 [4-[1-(oxetan-3-yl)-4-(trifluoromethyl)imidazol-2-yl]phenyl]methanol

To a stirred solution of 2-(4-cyclopropyl-6-methoxy-pyrimidin-5-yl)-5H-pyrrolo[3,2-d]pyrimidine (100 mg, 375 μmol) and 4-[1-(oxetan-3-yl)-4-(trifluoromethyl)imidazol-2-yl]benzaldehyde (111 mg, 375 μmol) in water (1 mL) and propan-2-ol (1 mL) under nitrogen atmosphere was added potassium carbonate (62 mg, 449 μmol) at 25° C. After the resulting mixture was stirred at 25° C. for 2 hours. The resulted mixture was extracted with ethyl acetate (3×30 mL). Tc combined organic layers were washed with brine (3×10 mL), dried over anhydrous sodium sulfate. After filtration, the filtrate was concentrated under reduced pressure. The residue was purified by silica gel column chromatography, eluted with 10% methanol in dichloromethane to give a crude product (50 mg). The crude product was further purified by reverse flash chromatography with the following conditions: Column: C18 spherical, 20-30 μm, 100A, 40 g; Mobile Phase A: 10 mM aq. NH₄HCOs, Mobile Phase B: MeCN; Flow rate: 50 mL/min; Gradient: 5% B to 5% B in 5 min, 5% B to 45% B in 13 min; 45% B to 45% B in 5 min; 45% B to 95% B in 5 min; Detector: UV 254 & 220 nm; RT: 20 min. The fractions were collected, concentrated under reduced pressure and then lyophilized overnight to afford [2-(4-cyclopropyl-6-methoxy-pyrimidin-5-yl)-5H-pyrrolo[3,2-d]pyrimidin-7-yl]-[4-[1-(oxetan-3-yl)-4-(trifluoromethyl)imidazol-2-yl]phenyl]methanol (45 mg, 79.8 μmol, 21% yield) as an off-white solid. MS: m/z=564.45 [M+H]⁺. ¹H NMR (400 MHz, Chloroform-d) δ 9.98 (s, 1H), 8.95 (s, 1H), 8.67 (s, 1H), 7.95 (s, 1H), 7.67 (d, J=8.0 Hz, 2H), 7.44 (d, J=8.0 Hz, 2H), 7.08 (s, 1H), 6.41 (s, 1H), 5.51-5.44 (m, 1H), 5.11-5.06 (m, 2H), 4.88-4.84 (m, 2H), 4.42 (br., 1H), 3.93 (s, 3H), 1.76-1.69 (m, 1H), 1.23-1.22 (m, 2H), 0.89-0.87 (m, 2H).

### 1.6 2-(4-cyclopropyl-6-methoxy-pyrimidin-5-yl)-7-[[4-[1-(oxetan-3-yl)-4-(trifluoromethyl)imidazol-2-yl]phenyl]methyl]-5H-pyrrolo[3,2-d]pyrimidine

To a stirred solution of [2-(4-cyclopropyl-6-methoxy-pyrimidin-5-yl)-5H-pyrrolo[3,2-d]pyrimidin-7-yl]-[4-[1-(oxetan-3-yl)-4-(trifluoromethyl)imidazol-2-yl]phenyl]methanol (40 mg, 71 μmol) and 2,2,2-trifluoroacetic acid (740 mg, 6.5 mmol, 0.5 mL) in chloroform (0.5 mL) under nitrogen atmosphere was added triethylsilane (12 mg, 71 μmol, 1 mL) at room temperature and then stirred at this temperature for 3 hours. The resulting mixture was concentrated under reduced pressure. The residue was purified by RP-Flash with the following conditions: Column: C18 spherical Column, 20-35 μm, 100A, 20 g; Mobile Phase A: Water (5 mM aq. NH₄HCO₃), Mobile Phase B: MeCN; Flow rate: 25 mL/min; Gradient: 5% B to 5% B in 5 min, 5% B to 70% B in 15 min, 70% B to 70% B in 2 min, 70% B to 95% B in 5 min; Detector: UV 254 & 210 nm; RT: 21 min. The collected fractions were combined, concentrated and lyophilized to give 2-(4-cyclopropyl-6-methoxy-pyrimidin-5-yl)-7-[[4-[1-(oxetan-3-yl)-4-(trifluoromethyl)imidazol-2-yl]phenyl]methyl]-5H-pyrrolo[3,2-d]pyrimidine (16 mg, 29.22 μmol, 41% yield) as an off-white solid. MS: m/z=548.20 [M+H]⁺. ¹H NMR (400 MHz, Methanol-d4) δ 8.98 (s, 1H), 8.64 (s, 1H), 8.27 (s, 1H), 7.77 (s, 1H), 7.53 (d, J=8.0 Hz, 2H), 7.41 (d, J=8.0 Hz, 2H), 5.55-5.50 (m, 1H), 5.00-4.95 (m, 2H), 4.84-4.83 (m, 2H), 4.30 (s, 2H), 3.94 (s, 3H), 1.64-1.59 (m, 1H), 1.31-1.15 (m, 2H), 0.92-0.87 (m, 2H).

### Example 55. 7-[[4-[1-methyl-4-(trifluoromethyl)imidazol-2-yl]phenyl]methyl]-2-[2-methyl-4-(trifluoromethyl)pyrazol-3-yl]-5H-pyrrolo[3,2-d]pyrimidine Compound 179

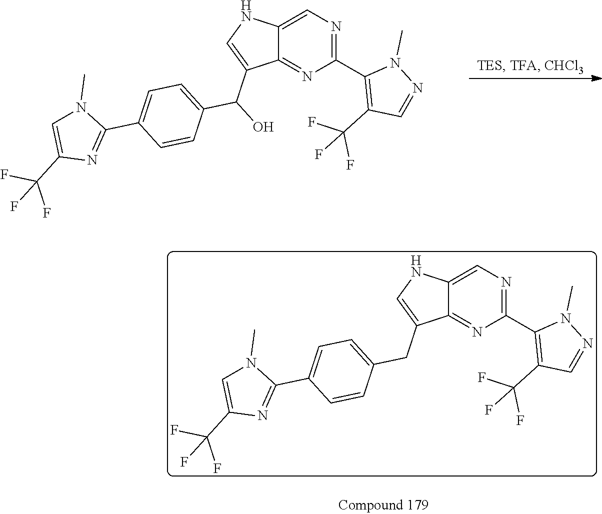 [SMILES: Cn1cc(C(F)(F)F)nc1-c1ccc(C(O)c2c[nH]c3cnc(-c4c(C(F)(F)F)cnn4C)nc23)cc1 ; "TES, TFA, CHCl3" ; Cn1cc(C(F)(F)F)nc1-c1ccc(Cc2c[nH]c3cnc(-c4c(C(F)(F)F)cnn4C)nc23)cc1]

To a solution of [4-[1-methyl-4-(trifluoromethyl)imidazol-2-yl]phenyl]-[2-[2-methyl-4-(trifluoromethyl)pyrazol-3-yl]-5H-pyrrolo[3,2-d]pyrimidin-7-yl]methanol (70 mg, 134 μmol) and triethylsilane (1.16 g, 10 mmol, 1.6 mL) in chloroform (1.6 mL) under nitrogen atmosphere was added 2,2,2-trifluoroacetic acid (1.18 g, 10.4 mmol, 0.8 mL) at 0° C. The reaction mixture was stirred at 25° C. for 3 hours. The reaction mixture was concentrated under reduced pressure. The residue was purified by Prep-TLC, eluted with 10% methanol in dichloromethane to afford a crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 5% B to 5% B in 3 min, 5% B to 50% B in 15 min, 50% B to 50% B in 5 min, 50% B to 95% B in 10 min, 95% B to 95% B in 2 min; Detector: UV 254 & 210 nm; RT: 20 min. The collected fractions were combined, concentrated and then lyophilized overnight to give 7-[[4-[1-methyl-4-(trifluoromethyl)imidazol-2-yl]phenyl]methyl]-2-[2-methyl-4-(trifluoromethyl)pyrazol-3-yl]-5H-pyrrolo[3,2-d]pyrimidine (33.9 mg, 67.07 μmol, 50% yield) as an off-white solid. MS: m/z=506.25 [M+H]⁺. ¹H NMR (400 MHz, Chloroform-d) δ 10.55 (s, 1H), 8.90 (s, 1H), 7.78 (s, 1H), 7.50 (d, J=8.0 Hz, 2H), 7.43-7.37 (m, 3H), 7.29 (s, 1H), 4.24 (s, 2H), 4.11 (s, 3H), 3.78 (s, 3H). ¹⁹F NMR (376 MHz, Chloroform-d) δ−−55.63, 62.34.

### Example 56. 5-(4-cyclopropyl-6-methoxy-pyrimidin-5-yl)-3-[[4-[1-cyclopropyl-4-(trifluoromethyl)imidazol-2-yl]phenyl]methyl]-1H-pyrazolo[4,3-d]pyrimidine Compound

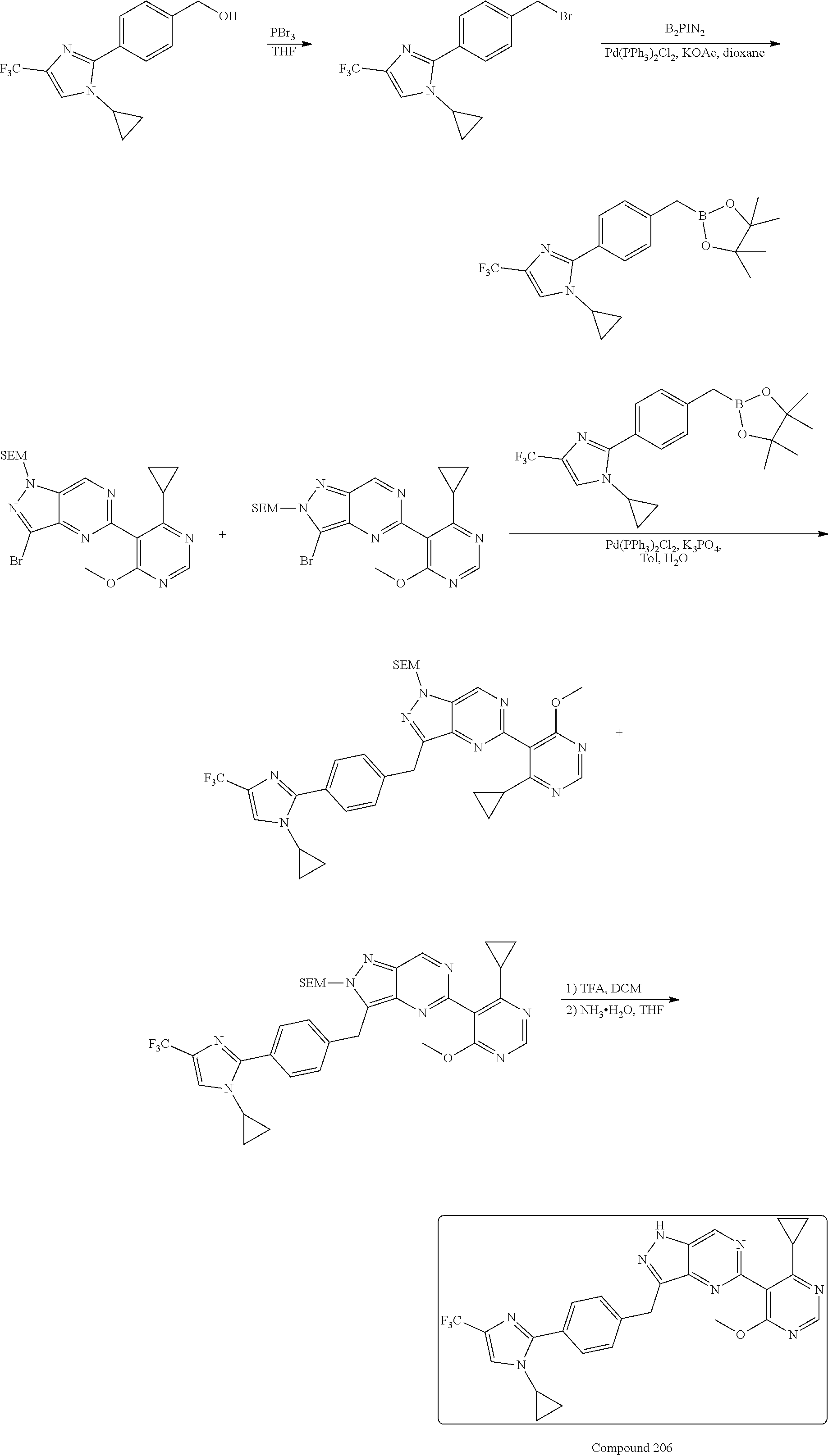

### 1.1 2-[4-(bromomethyl)phenyl]-1-cyclopropyl-4-(trifluoromethyl)imidazole

To a solution of [4-[1-cyclopropyl-4-(trifluoromethyl)imidazol-2-yl]phenyl]methanol (400 mg, 1.42 mmol) in THF (10 mL) was added phosphorus bromide (1.92 g, 7.09 mmol) at 0° C., naturally warmed up to 15° C. then was stirred at 15° C. for 2 hours. The resulted solution was quenched by saturated sodium bicarbonate aqueous solution (200 mL) and extracted by ethyl acetate (3×150 mL). The combined organic layers were dried over anhydrous sodium sulfate. After filtration, the filtrate was concentrated under reduced pressure. The residue was purified by silica gel column chromatography, eluted with 10%-25% ethyl acetate in petroleum ether to afford 2-[4-(bromomethyl)phenyl]-1-cyclopropyl-4-(trifluoromethyl)imidazole (420 mg, 1.22 mmol, 85% yield) as a yellow oil. MS: m/z=345.10, 347.10 [M+H]⁺. ¹H NMR (400 MHz, Chloroform-d) δ 7.90-7.83 (m, 2H), 7.55-7.47 (m, 2H), 7.38 (s, 1H), 4.56 (s, 2H), 3.57-3.51 (m, 1H), 1.19-1.03 (m, 2H), 0.96-0.91 (m, 2H).

### 1.2 1-cyclopropyl-2-[4-[(4,4,5,5-tetramethyl-1,3,2-dioxaborolan-2-yl)methyl]phenyl]-4-(trifluoromethyl)imidazole

To a solution of 2-[4-(bromomethyl)phenyl]-1-cyclopropyl-4-(trifluoromethyl)imidazole (370 mg, 1.07 mmol) in 1,4-dioxane (15 mL) were added 4,4,5,5-tetramethyl-2-(4,4,5,5-tetramethyl-1,3,2-dioxaborolan-2-yl)-1,3,2-dioxaborolane (408 mg, 1.6 mmol), bis(triphenylphosphine)palladium(II) chloride (75 mg, 107 μmol) and potassium acetate (316 mg, 3.22 mmol) at 15° C. The above mixture was stirred at 80° C. for 16 hours under nitrogen atmosphere. The resulted reaction was filtrated. The filtrate was concentrated under reduced pressure. The residue was purified by silica gel column chromatography, eluted with 15%-35% ethyl acetate in petroleum ether to afford 1-cyclopropyl-2-[4-[(4,4,5,5-tetramethyl-1,3,2-dioxaborolan-2-yl)methyl]phenyl]-4-(trifluoromethyl)imidazole (300 mg, 764.87 μmol, 71% yield) as a yellow solid. MS: m/z=393.30 [M+H]⁺. ¹H NMR (400 MHz, Chloroform-d) δ 7.75-7.72 (m, 2H), 7.36-7.27 (m, 3H), 3.54-3.48 (m, 1H), 2.37 (s, 2H), 1.25 (s, 12H), 1.13-1.03 (m, 2H), 0.95-0.87 (m, 2H).

### 1.3 2-[[5-(4-cyclopropyl-6-methoxy-pyrimidin-5-yl)-3-[[4-[1-cyclopropyl-4-(trifluoromethyl)imidazol-2-yl]phenyl]methyl]pyrazolo[4,3-d]pyrimidin-1-yl]methoxy]ethyl-trimethyl-silane & 2-[[5-(4-cyclopropyl-6-methoxy-pyrimidin-5-yl)-3-[[4-[1-cyclopropyl-4-(trifluoromethyl)imidazol-2-yl]phenyl]methyl]pyrazolo[4,3-d]pyrimidin-2-yl]methoxy]ethyl-trimethyl-silane

To a solution of 1-cyclopropyl-2-[4-[(4,4,5,5-tetramethyl-1,3,2-dioxaborolan-2-yl)methyl]phenyl]-4-(trifluoromethyl)imidazole (250 mg, 637.39 μmol) in toluene (5 mL) and water (1 mL) were added a mixture of 2-[[3-bromo-5-(4-cyclopropyl-6-methoxy-pyrimidin-5-yl)pyrazolo[4,3-d]pyrimidin-1-yl]methoxy]ethyl-trimethyl-silane & 2-[[3-bromo-5-(4-cyclopropyl-6-methoxy-pyrimidin-5-yl)pyrazolo[4,3-d]pyrimidin-2-yl]methoxy]ethyl-trimethyl-silane (304 mg, 637 μmol), bis(triphenylphosphine)palladium(II) chloride (44.7 mg, 63.7 μmol) and potassium phosphate (271 mg, 1.3 mmol) at 15° C. The above mixture was stirred at 90° C. for 16 hours under nitrogen atmosphere then was filtrated. The filtrate was concentrated under reduced pressure. The residue was purified by silica gel column chromatography, eluted with 15%-100% ethyl acetate in petroleum ether to give 2-[[5-(4-cyclopropyl-6-methoxy-pyrimidin-5-yl)-3-[[4-[1-cyclopropyl-4-(trifluoromethyl)imidazol-2-yl]phenyl]methyl]pyrazolo[4,3-d]pyrimidin-1-yl]methoxy]ethyl-trimethyl-silane & 2-[[5-(4-cyclopropyl-6-methoxy-pyrimidin-5-yl)-3-[[4-[1-cyclopropyl-4-(trifluoromethyl)imidazol-2-yl]phenyl]methyl]pyrazolo[4,3-d]pyrimidin-2-yl]methoxy]ethyl-trimethyl-silane (217 mg, crude, mixture of two isomers) as a white solid, which was used in the next step without further separated. MS: m/z=663.55 [M+H]⁺.

### 1.4. 5-(4-cyclopropyl-6-methoxy-pyrimidin-5-yl)-3-[[4-[1-cyclopropyl-4-(trifluoromethyl)imidazol-2-yl]phenyl]methyl]-1H-pyrazolo[4,3-d]pyrimidine

To a solution of 2,2,2-trifluoroacetic acid (2.5 mL) in dichloromethane (2.5 mL) was added a mixture of 2-[[5-(4-cyclopropyl-6-methoxy-pyrimidin-5-yl)-3-[[4-[[1-cyclopropyl-4-(trifluoromethyl)imidazol-2-yl]phenyl]methyl]pyrazolo[4,3-d]pyrimidin-1-yl]methoxy]ethyl-trimethyl-silane & 2-[[5-(4-cyclopropyl-6-methoxy-pyrimidin-5-yl)-3-[[4-[1-cyclopropyl-4-(trifluoromethyl)imidazol-2-yl]phenyl]methyl]pyrazolo[4,3-d]pyrimidin-2-yl]methoxy]ethyl-trimethyl-silane (217 mg, 327 μmol) at 15° C. The above solution was stirred at 15° C. for 2 hours then concentrated under reduced pressure. The residue was added tetrahydrofuran (2.5 mL) and ammonia solution (28% aqueous solution, 2.5 mL), then was stirred at 25° C. for 10 min. The resulted solution was concentrated under reduced pressure. The residue was purified RP-Flash chromatography with the following conditions: Column: C18 spherical, 20-35 um, 100A, 80 g; Mobile Phase A: 10 mM aq. ammonium bicarbonate, Mobile Phase B: acetonitrile; Flow rate: 60 mL/min; Gradient: 5% B to 5% B in 5 min, 5% B to 25% B in 5 min, 25% B to 55% B in 30 min, 55% B to 95% B in 5 min; Detector: UV 254 & 220 nm; RT: 26 min. The collected fractions were concentrated under reduced pressure and then lyophilized overnight to afford to give 5-(4-cyclopropyl-6-methoxy-pyrimidin-5-yl)-3-[[4-[I-cyclopropyl-4-(trifluoromethyl)imidazol-2-yl]phenyl]methyl]-1H-pyrazolo[4,3-d]pyrimidine (64.2 mg, 120.56 μmol, 36% yield) as a white solid. MS: m/z=533.20 [M+H]⁺. ¹H NMR (400 MHz, DMSO-d₆) δ 13.79 (br., 1H), 9.44 (s, 1H), 8.70 (s, 1H), 7.90 (s, 1H), 7.80 (d, J=8.0 Hz, 2H), 7.49 (d, J=8.4 Hz, 2H), 4.46 (s, 2H), 3.84 (s, 3H), 3.73-3.67 (m, 1H), 1.64-1.58 (m, 1H), 1.07-1.03 (m, 2H), 1.00-0.94 (m, 2H), 0.91-0.88 (m, 2H), 0.86-0.81 (m, 2H). ¹⁹F NMR (376 MHz, DMSO-d₆) δ −60.88.

### Example 57. 5-(4-cyclopropyl-6-methoxy-pyrimidin-5-yl)-3-[[4-[1-(oxetan-3-yl)-4-(trifluoromethyl)imidazol-2-yl]phenyl]methyl]-1H-pyrazolo[4,3-d]pyrimidine Compound 133

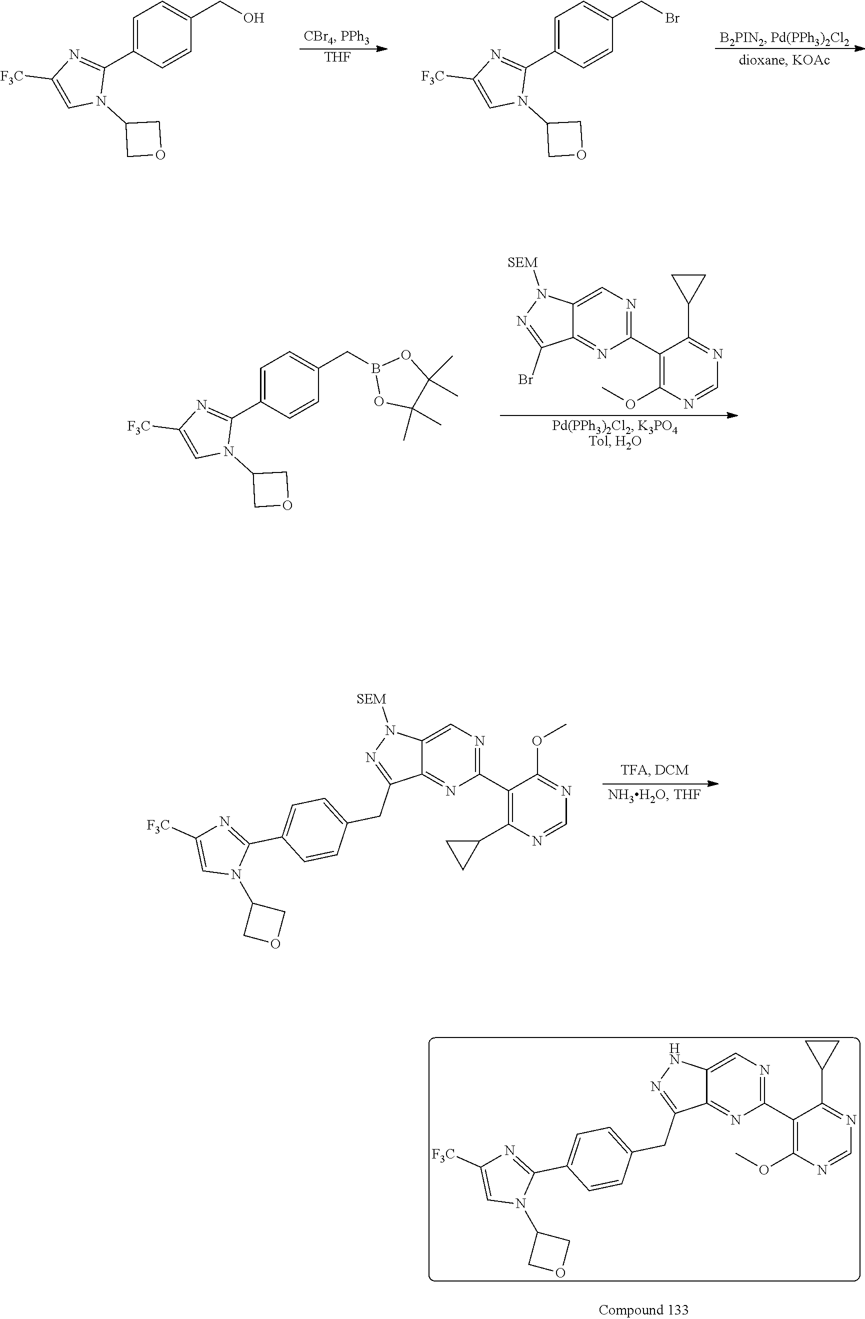

### 1.1 2-[4-(bromomethyl)phenyl]-1-(oxetan-3-yl)-4-(trifluoromethyl)imidazole

To a stirred solution of [4-[1-(oxetan-3-yl)-4-(trifluoromethyl)imidazol-2-yl]phenyl]methanol (370 mg, 1.24 mmol) in tetrahydrofuran (5 mL) were added carbon tetrabromide (41 mg, 1.24 mmol) and triphenylphosphane (342 mg, 1.30 mmol) at 25° C. under nitrogen atmosphere and then stirred at this temperature 2 hours. The resulting mixture was diluted with ethyl acetate (50 mL). The reaction mixture was washed with brine (20 mL), dried over anhydrous sodium sulfate. After filtration, the filtrate was concentrated under reduced pressure. The residue was purified by silica gel column chromatography, eluted with 1%-20% ethyl acetate in petroleum ether to give 2-[4-(bromomethyl)phenyl]-1-(oxetan-3-yl)-4-(trifluoromethyl)imidazole (270 mg, 747.60 μmol, 60% yield) as a light yellow solid. MS: m/z=361.00, 363.05 [M+H]⁺. ¹H NMR (400 MHz, Chloroform-d) δ 7.92 (s, 1H), 7.54 (d, J=8.0 Hz, 2H), 7.45 (d, J=8.0 Hz, 2H), 5.53-5.45 (m, 1H), 5.13-5.08 (m, 2H), 4.89-4.84 (m, 2H), 4.55 (s, 2H).

### 1.2 1-(oxetan-3-yl)-2-[4-[(4,4,5,5-tetramethyl-1,3,2-dioxaborolan-2-yl)methyl]phenyl]-4-(trifluoromethyl)imidazole

To a stirred solution of 2-[4-(bromomethyl)phenyl]-1-(oxetan-3-yl)-4-(trifluoromethyl)imidazole (237 mg, 656.2 μmol) and 4,4,5,5-tetramethyl-2-(4,4,5,5-tetramethyl-1,3,2-dioxaborolan-2-yl)-1,3,2-dioxaborolane (250 mg, 984 μmol) in 1,4-dioxane (2 mL) was added potassium acetate (193 mg, 1.97 mmol) and bis(triphenylphosphine)palladium(II) chloride (46 mg, 65.6 μmol) at 25° C. under nitrogen atmosphere. The resulting mixture was stirred at 25° C. for 18 hours under nitrogen atmosphere then diluted with ethyl acetate (50 mL). The resulting mixture was washed with brine (2×25 mL), dried over anhydrous sodium sulfate. After filtration, the filtrate was concentrated under reduced pressure. The residue was purified by silica gel column chromatography, eluted with 1%-25% petroleum ether in ethyl acetate to give 1-(oxetan-3-yl)-2-[4-[(4,4,5,5-tetramethyl-1,3,2-dioxaborolan-2-yl)methyl]phenyl]-4-(trifluoromethyl)imidazole (177 mg, 433.59 μmol, 66% yield) as a off-white solid. MS: m/z=409.10 [M+H]⁺.

### 1.3 2-[[5-(4-cyclopropyl-6-methoxy-pyrimidin-5-yl)-3-[[4-[1-(oxetan-3-yl)-4-(trifluoromethyl)imidazol-2-yl]phenyl]methyl]pyrazolo[4,3-d]pyrimidin-1-yl]methoxy]ethyl-trimethyl-silane

To a stirred solution of 1-(oxetan-3-yl)-2-[4-[(4,4,5,5-tetramethyl-1,3,2-dioxaborolan-2-yl)methyl]phenyl]-4-(trifluoromethyl)imidazole (137 mg, 336 μmol) and 2-[[3-bromo-5-(4-cyclopropyl-6-methoxy-pyrimidin-5-yl)pyrazolo[4,3-d]pyrimidin-1-yl]methoxy]ethyl-trimethyl-silane (160 mg, 336 μmol) in water (0.3 mL) and toluene (1.5 mL) were added potassium phosphate (142 mg, 671 μmol) and bis(triphenylphosphine)palladium(II) chloride (24 mg, 33.5 μmol) at 25° C. under nitrogen atmosphere. The resulting mixture was stirred at 90° C. for 16 hours under nitrogen atmosphere then diluted with ethyl acetate (30 mL). The resulting mixture was washed with brine (15 mL), dried over anhydrous sodium sulfate. After filtration, the filtrate was concentrated under reduced pressure. The residue was purified by silica gel column chromatography, eluted with 50% petroleum ether in ethyl acetate to give 2-[[5-(4-cyclopropyl-6-methoxy-pyrimidin-5-yl)-3-[[4-[1-(oxetan-3-yl)-4-(trifluoromethyl)imidazol-2-yl]phenyl]methyl]pyrazolo[4,3-d]pyrimidin-1-yl]methoxy]ethyl-trimethyl-silane (75 mg, 110.49 μmol, 32% yield) as a yellow solid. MS: m/z=679.20 [M+H]⁺.

### 1.4 5-(4-cyclopropyl-6-methoxy-pyrimidin-5-yl)-3-[[4-[1-(oxetan-3-yl)-4-(trifluoromethyl)imidazol-2-yl]phenyl]methyl]-1H-pyrazolo[4,3-d]pyrimidine

To a stirred solution of 2-[[5-(4-cyclopropyl-6-methoxy-pyrimidin-5-yl)-3-[[4-[1-(oxetan-3-yl)-4-(trifluoromethyl)imidazol-2-yl]phenyl]methyl]pyrazolo[4,3-d]pyrimidin-1-yl]methoxy]ethyl-trimethyl-silane (75 mg, 110.49 μmol) in dichloromethane (0.50 mL) was added 2,2,2-trifluoroacetic acid (0.74 g, 6.49 mmol, 0.50 mL) at 25° C. under nitrogen atmosphere and then stirred at this temperature 1 hour. The resulted mixture was concentrated under reduced pressure. To the stirred solution of the residue in tetrahydrofuran (0.50 mL) was added ammonium hydroxide (28% NH₃ in H₂O, 0.50 mL) at 25° C. under nitrogen atmosphere and stirred at this temperature for 1 hour. The resulted mixture was concentrated under reduced pressure. The obtained product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 35 mL/min; Gradient: 5% B to 5% B in 5 min, 5% B to 95% B in 19 min; Detector: UV 254 & 210 nm; RT: 14 min. The collected fractions were combined, concentrated under reduced pressure and lyophilized to give 5-(4-cyclopropyl-6-methoxy-pyrimidin-5-yl)-3-[[4-[1-(oxetan-3-yl)-4-(trifluoromethyl)imidazol-2-yl]phenyl]methyl]-1H-pyrazolo[4,3-d]pyrimidine (34.7 mg, 63.26 μmol, 57% yield) as a light yellow solid. MS: m/z=549.20 [M+H]⁺. ¹H NMR (400 MHz, Chloroform-d) δ 11.73 (br., 1H), 9.27 (s, 1H), 8.73 (s, 1H), 7.93 (s, 1H), 7.60 (d, J=8.0 Hz, 2H), 7.43 (d, J=8.0 Hz, 2H), 5.52-5.45 (m, 1H), 5.10-5.02 (m, 2H), 4.88-4.81 (m, 2H), 4.56 (s, 2H), 3.94 (s, 3H), 1.68-1.62 (m, 1H), 1.32-1.20 (m, 2H), 0.89-0.86 (m, 2H). ¹⁹F NMR (376 MHz, Chloroform-d) δ −62.73.

### Example 58. 1-(4-((2-(4-cyclopropyl-6-methoxypyrimidin-5-yl)-5H-pyrrolo[3,2-d]pyrimidin-7-yl)methyl)phenyl)-5-methyl-1H-pyrazole-3-carbonitrile Compound 197

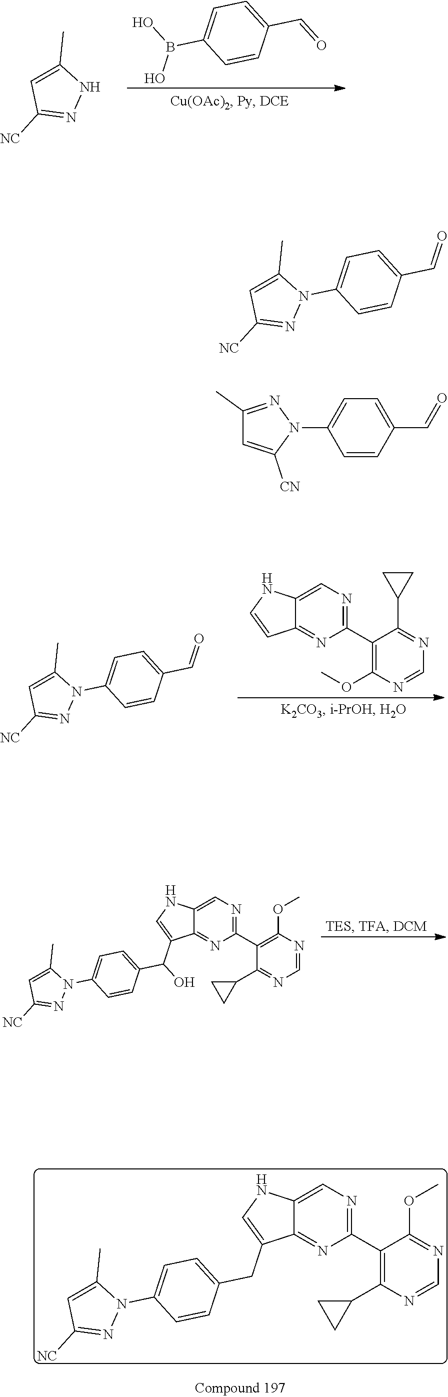

### 1.1 1-(4-formylphenyl)-5-methyl-1H-pyrazole-3-carbonitrile & 1-(4-formylphenyl)-3-methyl-1H-pyrazole-5-carbonitrile

To a stirred solution of 5-methyl-1H-pyrazole-3-carbonitrile (1 g, 9.34 mmol) and (4-formylphenyl)boronic acid (2.80 g, 18.67 mmol) in 1,2-dichloroethane (50 mL) were added cupric acetate (3.39 g, 18.67 mmol) and pyridine (3.69 g, 46.68 mmol, 3.78 mL) at room temperature, the resulting mixture was stirred at 60° C. for 16 h under air atmosphere. Major product was observed by LCMS. The resulted mixture was concentrated under reduced pressure. The residue was diluted with ethyl acetate (80 mL), washed with water (30 mL) and brine (30 mL). The organic layer was dried over anhydrous sodium sulfate and filtration. The filtrate was concentrated under reduced pressure. The residue was purified by silica gel column chromatography, eluted with 0%-80% ethyl acetate in petroleum ether to give two isomers. The small polar isomer fractions were concentrated under reduced pressure to give 1-(4-formylphenyl)-5-methyl-1H-pyrazole-3-carbonitrile (630 mg, 2.98 mmol, 32% yield) as off-white solid. MS: m/z=212.20 [M+H]⁺. ¹H NMR (400 MHz, DMSO-d₆) δ 10.11 (s, 1H), 8.08-8.04 (m, 2H), 7.71-7.67 (m, 2H), 6.65 (s, 1H), 2.46 (s, 3H).

The big polar isomer fractions were concentrated under reduced pressure to give 1-(4-formylphenyl)-3-methyl-1H-pyrazole-5-carbonitrile (152 mg, 0.72 mmol, 8% yield) as off-white solid. MS: m/z=212.05 [M+H]⁺. ¹H NMR (400 MHz, DMSO-d₆) δ 10.08 (s, 1H), 8.05 (d, J=8.4 Hz, 2H), 7.96 (d, J=8.4 Hz, 2H), 6.90 (s, 1H), 2.43 (s, 3H).

### 1.2 1-(4-((2-(4-cyclopropyl-6-methoxypyrimidin-5-yl)-5H-pyrrolo[3,2-d]pyrimidin-7-yl)(hydroxy)methyl)phenyl)-5-methyl-1H-pyrazole-3-carbonitrile

To a stirred solution of 2-(4-cyclopropyl-6-methoxy-pyrimidin-5-yl)-5H-pyrrolo[3,2-d]pyrimidine (106 mg, 398 μmol) and 1-(4-formylphenyl)-5-methyl-1H-pyrazole-3-carbonitrile (84 mg, 398 μmol) in isopropyl alcohol (3 mL) were added potassium carbonate (66 mg, 477 μmol) and water (3 mL) at room temperature under nitrogen atmosphere, the resulting mixture was stirred at 60° C. for 16 h. Major product was observed by LCMS. The resulted mixture was diluted with water (10 mL), extracted with ethyl acetate (3×15 mL). The organic layer was dried over anhydrous sodium sulfate and filtration. The filtrate was concentrated under reduced pressure. The residue was purified by RP-Flash chromatography with the following conditions: Column: C18 spherical, 20-30 um, 100A, 40 g; Mobile Phase A: Water (10 mM aq. NH₄HCO₃), Mobile Phase B: MeCN; Flow rate: 40 mL/min; Gradient: 5% B to 55% B in 15 min; 55% B to 55% B in 3 min; 55% B to 95% B in 5 min; Detector: UV 254 & 220 nm; RT: 16 min. The collected fractions were combined, concentrated and then lyophilized overnight to give 1-(4-((2-(4-cyclopropyl-6-methoxypyrimidin-5-yl)-5H-pyrrolo[3,2-d]pyrimidin-7-yl)(hydroxy)methyl)phenyl)-5-methyl-1H-pyrazole-3-carbonitrile (50 mg, 104.49 μmol, 26% yield) as off-white solid. MS: m/z=479.15 [M+H]⁺.

### 1.3 1-(4-((2-(4-cyclopropyl-6-methoxypyrimidin-5-yl)-5H-pyrrolo[3,2-d]pyrimidin-7-yl)methyl)phenyl)-5-methyl-1H-pyrazole-3-carbonitrile

To a solution of 1-(4-((2-(4-cyclopropyl-6-methoxypyrimidin-5-yl)-5H-pyrrolo[3,2-d]pyrimidin-7-yl)(hydroxy)methyl)phenyl)-5-methyl-1H-pyrazole-3-carbonitrile (90 mg, 188 μmol) and triethylsilane (2 mL) in dichloromethane (2 mL) was added trifluoroacetic acid (1 mL) dropwise at 25° C., and then stirred at this temperature for 16 h under nitrogen atmosphere. The mixture was concentrated under reduced pressure. The residue was purified by RP-Flash with the following conditions: Column: C18 spherical, 20-30 um, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 45% B in 15 min, 45% B to 45% B in 3 min, 45% B to 95% B in 8 min; Detector: UV 254 & 220 nm; RT: 21 min. The collected fractions were combined, concentrated and then lyophilized overnight to give 1-(4-((2-(4-cyclopropyl-6-methoxypyrimidin-5-yl)-5H-pyrrolo[3,2-d]pyrimidin-7-yl)methyl)phenyl)-5-methyl-1H-pyrazole-3-carbonitrile (32.5 mg, 70.27 μmol, 37% yield) as an off-white solid. MS: m/z=463.25 [M+H]⁺. ¹H NMR (400 MHz, Chloroform-d) 9.15-9.02 (m, 2H), 8.68 (s, 1H), 7.51 (d, J=8.4 Hz, 2H), 7.42 (s, 1H), 7.36 (d, J=8.4 Hz, 2H), 6.60 (s, 1H), 4.33 (s, 2H), 3.95 (s, 3H), 2.35 (s, 3H), 1.80-1.70 (m, 1H), 1.30-1.20 (m, 2H), 0.91-0.85 (m, 2H).

### Example 59. 1-[4-[[2-(4-cyclopropyl-6-methoxy-pyrimidin-5-yl)-5-methyl-pyrrolo[3,2-d]pyrimidin-7-yl]methyl]phenyl]-5-methyl-pyrazole-3-carbonitrile Compound 107

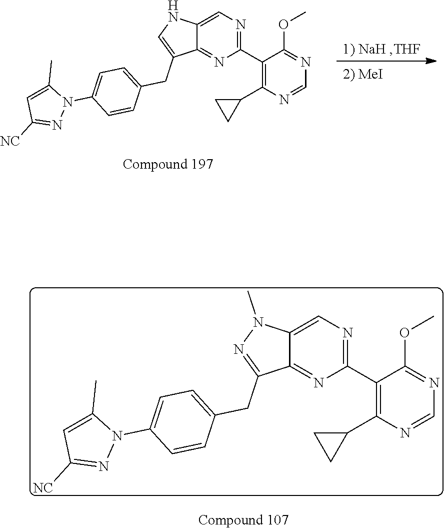

### 1.1 1-[4-[[2-(4-cyclopropyl-6-methoxy-pyrimidin-5-yl)-5-methyl-pyrrolo[3,2-d]pyrimidin-7-yl]methyl]phenyl]-5-methyl-pyrazole-3-carbonitrile

To a solution of 1-[4-[[2-(4-cyclopropyl-6-methoxy-pyrimidin-5-yl)-5H-pyrrolo[3,2-d]pyrimidin-7-yl]methyl]phenyl]-5-methyl-pyrazole-3-carbonitrile (44 mg, 95.13 μmol) in THF (4 mL) was added sodium hydride (7.6 mg, 190 μmol, 60% dispersion in oil) at 0° C. under nitrogen atmosphere. The resulting mixture was stirred at 0° C. for 1 hour. Then iodomethane (20 mg, 143 μmol, 8.9 μL) was added dropwise at 0° C. The resulting mixture was stirred at 20° C. for 1.5 hours. The resulted mixture was purified by Prep-TLC directly to give a crude product. The crude product was purified by RP-Flash with the following conditions: C18 spherical Column, 20-35 um, 100A, 40 g; Mobile Phase A: Water (5 mM aq. NH₄HCO₃), Mobile Phase B: MeCN; Flow rate: 35 mL/min; Gradient: 5% B to 5% B in 5 min, 5% B to 55% B in 10 min. 55% B to 55% B in 5 min, 55% B to 95% B in 5 min; Detector: UV 254 & 210 nm; RT: 17 min. The collected fractions were combined, concentrated and then lyophilized overnight to give 1-[4-[[2-(4-cyclopropyl-6-methoxy-pyrimidin-5-yl)-5-methyl-pyrrolo[3,2-d]pyrimidin-7-yl]methyl]phenyl]-5-methyl-pyrazole-3-carbonitrile (23.1 mg, 48.48 μmol, 51% yield) as an off-white solid. MS: m/z=477.20 [M+H]⁺. ¹H NMR (400 MHz, Chloroform) δ 9.01 (s, 1H), 8.69 (s, 1H), 7.51 (d, J=8.4 Hz, 2H), 7.36 (d, J=8.4 Hz, 2H), 7.20 (s, 1H), 6.60 (s, 1H), 4.31 (s, 2H), 3.96 (s, 3H), 3.94 (s, 3H), 2.35 (s, 3H), 1.76-1.68 (m, 1H), 1.30-1.22 (m, 2H), 0.91-0.83 (m, 2H).

### Example 60. 5-(4-cyclopropyl-6-methoxy-pyrimidin-5-yl)-3-[4-[1-methyl-4-(trifluoromethyl)imidazol-2-yl]phenoxy]-1H-pyrrolo[2,3-c]pyridine Compound 167

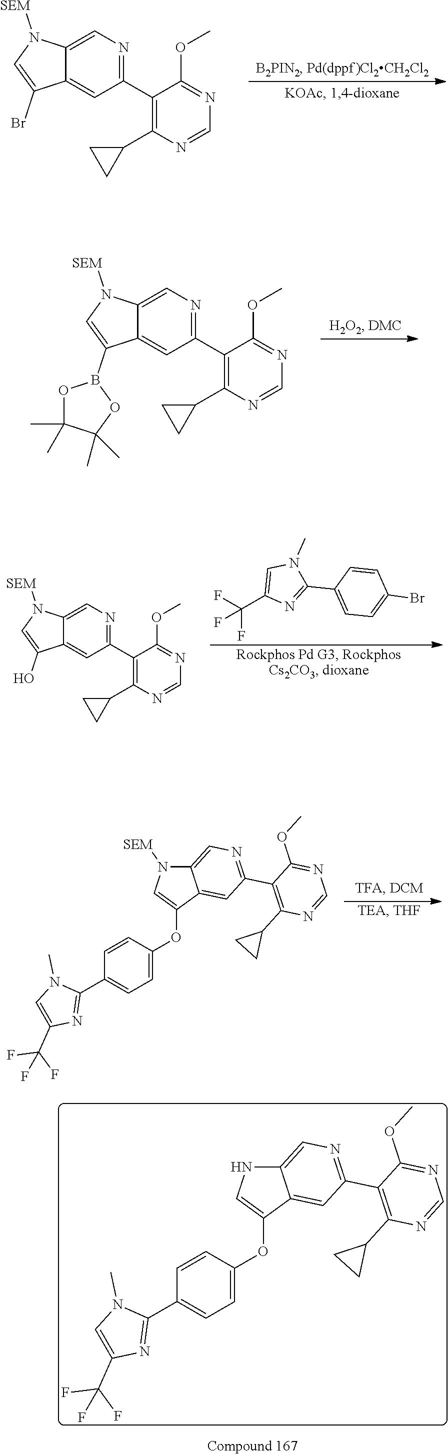

### 1.1 2-[[5-(4-cyclopropyl-6-methoxy-pyrimidin-5-yl)-3-(4,4,5,5-tetramethyl-1,3,2-dioxaborolan-2-yl)pyrrolo[2,3-c]pyridin-1-yl]methoxy]ethyl-trimethyl-silane

To a stirred mixture of 2-[[3-bromo-5-(4-cyclopropyl-6-methoxy-pyrimidin-5-yl)pyrrolo[2,3-c]pyridin-1-yl]methoxy]ethyl-trimethyl-silane (1 g, 2.10 mmol) and bis(pinacolato)diboron (801 mg, 3.15 mmol) in 1,4-dioxane (20 mL) were added potassium acetate (413 mg, 4.2 mmol) and 1,1′-bis(diphenylphosphino)ferrocene-palladium(II)dichloride dichloromethane complex (172 mg, 210 μmol) under nitrogen atmosphere at 25° C. The resulting mixture was stirred at 80° C. for 16 hours. The reaction was cooled to mom temperature, then diluted with water (50 mL), extracted with dichloromethane (3×20 mL). The combined organic layers were washed with brine (3×20 mL), dried over anhydrous sodium sulfate. After filtration, the filtrate was concentrated under reduced pressure. The resulted residue was purified by RP-Flash with the following conditions: Column: C18 spherical Column, 20-35 um, 100A, 80 g; Mobile Phase A: Water (5 mM aq. NH₄HCO₃). Mobile Phase B: MeCN; Flow rate: 60 mL/min; Gradient: 5% B to 5% B in 5 min, 5% B to 63% B in 10 min, 63% B to 63% B in 5 min, 63% B to 95% B in 5 min; Detector: UV 254 & 210 nm; RT: 20 min. The collected fractions were combined, concentrated and then lyophilized to give 2-[[5-(4-cyclopropyl-6-methoxy-pyrimidin-5-yl)-3-(4,4,5,5-tetramethyl-1,3,2-dioxaborolan-2-yl)pyrrolo[2,3-c]pyridin-1-yl]methoxy]ethyl-trimethyl-silane (440 mg, 842.08 μmol, 40% yield) as an off-white solid. MS: m/z=524.10 [M+H]⁺. ¹H NMR (400 MHz, Chloroform-d) δ 9.05 (s, 1H), 8.66 (s, 1H), 8.00 (s, 1H), 7.80 (s, 1H), 5.60 (s, 2H), 3.94 (s, 3H), 3.62-3.52 (m, 2H), 1.83-1.77 (m, 1H), 1.38 (s, 12H), 1.23-1.20 (m, 2H), 0.97-0.93 (m, 2H), 0.91-0.85 (m, 2H), −0.01 (s, 9H).

### 1.2 5-(4-cyclopropyl-6-methoxy-pyrimidin-5-yl)-1-(2-trimethylsilylethoxymethyl)pyrrolo[2,3-c]pyridin-3-ol

To a stirred mixture of 2-[[5-(4-cyclopropyl-6-methoxy-pyrimidin-5-yl)-3-(4,4,5,5-tetramethyl-1,3,2-dioxaborolan-2-yl)pyrrolo[2,3-c]pyridin-1-yl]methoxy]ethyl-trimethyl-silane (280 mg, 536 μmol) in dimethylcarbonate (5 mL) was added hydrogen peroxide (243 mg, 2.14 mmol, 30% in H₂O) under nitrogen atmosphere at 25° C. The resulting mixture was stirred for 16 hours at 25° C. The reaction was quenched by the addition of saturated sodium thiosulfate aqueous (20 mL). The resulting mixture was extracted with ethyl acetate (3×5 mL), dried over anhydrous sodium sulfate. After filtration, the filtrate was concentrated under reduced pressure.

The resulted residue was purified by RP-Flash with the following conditions: Column: C18 spherical Column, 20-35 um, 100A, 80 g; Mobile Phase A: Water (5 mM aq. NH₄HCO₃), Mobile Phase B: MeCN; Flow rate: 60 mL/min; Gradient: 5% B to 40% B in 15 min, 40% B to 40% B in 5 min, 40% B to 60% B in 8 min, 60% B to 95% B in 10 min; Detector: UV 254 & 210 nm; RT: 18 min. The collected fractions were combined, concentrated and then lyophilized to give 5-(4-cyclopropyl-6-methoxy-pyrimidin-5-yl)-1-(2-trimethylsilylethoxymethyl)pyrrolo[2,3-c]pyridin-3-ol (100 mg, 242.39 μmol, 45% yield) as an off-white solid. MS: m/z=413.20 [M+H]⁺.

### 1.3 2-[[5-(4-cyclopropyl-6-methoxy-pyrimidin-5-yl)-3-[4-[1-methyl-4-(trifluoromethyl)imidazol-2-yl]phenoxy]pyrrolo[2,3-c]pyridin-1-yl]methoxy]ethyl-trimethyl-silane

To a stirred mixture of 5-(4-cyclopropyl-6-methoxy-pyrimidin-5-yl)-1-(2-trimethylsilylethoxymethyl)pyrrolo[2,3-c]pyridin-3-ol (100 mg, 242.4 μmol) and cesium carbonate (237 mg, 727 μmol) in 1,4-dioxane (3 mL) was added 2-(4-bromophenyl)-1-methyl-4-(trifluoromethyl)imidazole (111 mg, 364 μmol) and di-tert-butyl(2′,4′,6′-triisopropyl-3-methoxy-6-methyl-[1,1′-biphenyl]-2-yl)phosphine (11 mg, 24 μmol) and methanesulfonato(2-(di-t-butylphosphino)-3-methoxy-6-methyl-2′,4′,6′-tri-i-propyl-1,1′-biphenyl)(2′-amino-1,1′-biphenyl-2-yl)palladium(II) (20 mg, 24 μmol) under nitrogen atmosphere at 25° C. The resulting mixture was stirred at 100° C. for 16 hours. The reaction was cooled down to room temperature, then diluted with water (10 mL), extracted with dichloromethane (3×5 mL). The combined organic layers were washed with brine (10 mL), dried over anhydrous sodium sulfate. After filtration, the filtrate was concentrated under reduced pressure. The resulted residue was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 66% B in 8 min, 66% B to 66% B in 4 min, 63% B to 95% B in 8 min; Detector: UV 254 & 210 nm; RT: 16 min. The collected fractions were combined, concentrated and then lyophilized to give 2-[[5-(4-cyclopropyl-6-methoxy-pyrimidin-5-yl)-3-[4-[1-methyl-4-(trifluoromethyl)imidazol-2-yl]phenoxy]pyrrolo[2,3-c]pyridin-1-yl]methoxy]ethyl-trimethyl-silane (15 mg, 23.56 μmol, 10% yield) as an off-white solid. MS: m/z=637.30 [M+H]⁺. ¹H NMR (400 MHz, Chloroform-d) δ 9.10 (s, 1H), 8.62 (s, 1H), 7.63-7.55 (m, 2H), 7.44 (s, 1H), 7.31 (s, 2H), 7.20-7.11 (m, 2H), 5.60 (s, 2H), 3.90 (s, 3H), 3.77 (s, 3H), 3.67-3.58 (m, 2H), 1.79-1.73 (m, 1H), 1.20-1.16 (m, 2H), 1.03-0.94 (m, 2H), 0.88-0.83 (m, 2H), 0.00 (s, 9H).

### 1.4 5-(4-cyclopropyl-6-methoxy-pyrimidin-5-yl)-3-[4-[1-methyl-4-(trifluoromethyl)imidazol-2-yl]phenoxy]-1H-pyrrolo[2,3-c]pyridine

To a stirred solution of 2-[[5-(4-cyclopropyl-6-methoxy-pyrimidin-5-yl)-3-[4-[1-methyl-4-(trifluoromethyl)imidazol-2-yl]phenoxy]pyrrolo[2,3-c]pyridin-1-yl]methoxy]ethyl-trimethyl-silane (5 mg, 7.85 μmol) in methylene chloride (1 mL) was added trifluoroacetic acid (1 mL) and then stirred for 2 hours at 25° C. The resulted mixture was concentrated under reduced pressure to afford a residue. Triethylamine (1 mL) and tetrahydrofuran (1 mL) were added in the above residue and the resulted mixture was stirred for 30 min at 25° C. then concentrated under reduced pressure. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 65% B in 20 min, 65% B to 65% B in 4 min, 65% B to 95% B in 5 min; Detector: UV 254 & 210 nm; RT: 26 min. The collected fractions were combined, concentrated and then lyophilized to give 5-(4-cyclopropyl-6-methoxy-pyrimidin-5-yl)-3-[4-[1-methyl-4-(trifluoromethyl)imidazol-2-yl]phenoxy]-1H-pyrrolo[2,3-c]pyridine (2.7 mg, 5.33 μmol, 68% yield) as an off-white solid. MS: m/z=507.40 [M+H]⁺. ¹H NMR (400 MHz, Methanol-d₄) δ 8.83 (s, 11H), 8.55 (s, 1H), 7.68 (s, 1H), 7.63-7.60 (m, 3H), 7.34 (s, 1H), 7.18 (d, J=8.4 Hz, 2H), 3.88 (s, 3H), 3.77 (s, 3H), 1.76-1.70 (s, 1H), 1.16-1.04 (m, 2H), 0.91-0.84 (m, 2H). ¹⁹F NMR (376 MHz, Methanol-d₄) δ −63.95.

### Example 61. 2-(4-cyclopropyl-2-methoxy-3-pyridyl)-7-[[4-[1-methyl-4-(trifluoromethyl)imidazol-2-yl]phenyl]methyl]-5H-pyrrolo[3,2-d]pyrimidine Compound 152

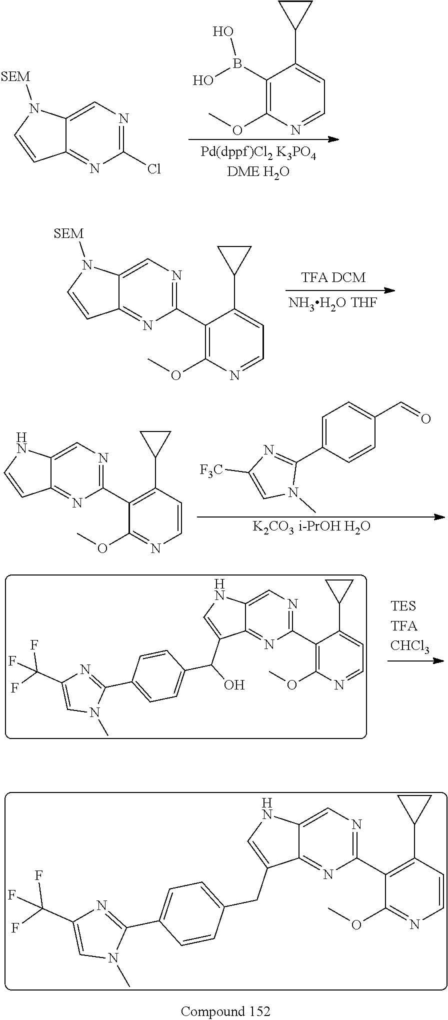

### 1.12 [[2-(4-cyclopropyl-2-methoxy-3-pyridyl)pyrrolo[3,2-d]pyrimidin-5-yl]methoxy]ethyl-trimethyl-silane

To a solution of 2-[(2-chloropyrrolo[3,2-d]pyrimidin-5-yl)methoxy]ethyl-trimethyl-silane (0.63 g, 2.22 mmol) and (4-cyclopropyl-2-methoxy-3-pyridyl)boronic acid (428 mg, 2.2 mmol) in 1,2-dimethoxy-ethan (10 mL) and water (2 mL) were added potassium phosphate (1.41 g, 6.66 mmol) and [1,1′-Bis(diphenylphosphino)ferrocene]dichloropalladium(II) (182 mg, 222 μmol). The mixture solution was stirred at 100° C. for 16 hours under nitrogen atmosphere. The resulted mixture was concentrated under reduced pressure. The residue was purified by silica gel column chromatography, eluted with petroleum ether:ethyl acetate=4:1 to afford 2-[[2-(4-cyclopropyl-2-methoxy-3-pyridyl)pyrrolo[3,2-d]pyrimidin-5-yl]methoxy]ethyl-trimethyl-silane (0.5 g, 1.26 mmol, 56% yield) as a yellow solid. MS: m/z=397.15 [M+H]⁺. ¹H NMR (400 MHz, Chloroform-d) δ 9.16 (s, 11H), 8.12 (d, J=5.6 Hz, 11H), 7.65 (d, J=2.8 Hz, 1H), 6.82 (d, J=2.8 Hz, 1H) 6.44 (d, J1=5.6 Hz, 1H), 5.61 (s, 2H), 3.92 (s, 3H), 3.62-3.55 (m, 2H), 1.67-1.47 (m, 1H), 1.03-0.93 (m, 2H), 0.86-0.79 (m, 4H), 0.00 (s, 9H).

### 1.2 2-(4-cyclopropyl-2-methoxy-3-pyridyl)-5H-pyrrolo[3,2-d]pyrimidine

A solution of 2-[[2-(4-cyclopropyl-2-methoxy-3-pyridyl)pyrrolo[3,2-d]pyrimidin-5-yl]methoxy]ethyl-trimethyl-silane (0.5 g, 1.26 mmol) in DCM (4 mL) and trifluoroacetic acid (4 mL) was stirred for 2.5 hours at room temperature. The reaction solution was concentrated under reduced pressure to give a residue. To the residue was added ammonium hydroxide (4 mL, 28% aqueous solution) and tetrahydrofuran (4 mL). The resulting mixture was stirred for another 1 hours at room temperature then concentrated under reduced pressure. The residue was purified by silica gel column chromatography, eluted with methanol in dichloromethane (0%-15%) to afford 2-(4-cyclopropyl-2-methoxy-3-pyridyl)-5H-pyrrolo[3,2-d]pyrimidine (270 mg, 1.01 mmol, 80% yield) as a light red solid. MS: m/z=267.25 [M+H]⁺. ¹H NMR (400 MHz, Methanol-d₄) δ 8.99 (s, 1H), 8.10 (d, J=5.6 Hz, 1H), 7.95 (d, J=3.2 Hz, 1H), 6.71 (d, J=3.2 Hz, 1H), 6.59 (d, J=5.6 Hz, 1H), 3.84 (s, 3H), 1.54-1.43 (m, 1H), 0.94-0.77 (m, 2H).

### 1.3 [2-(4-cyclopropyl-2-methoxy-3-pyridyl)-5H-pyrrolo[3,2-d]pyrimidin-7-yl]-[4-[1-methyl-4-(trifluoromethyl)imidazol-2-yl]phenyl]methanol

To a stirred solution of 2-(4-cyclopropyl-2-methoxy-3-pyridyl)-5H-pyrrolo[3,2-d]pyrimidine (0.27 g, 1.01 mmol) in isopropyl alcohol (2 mL) and water (2 mL) was added potassium carbonate (168 mg, 1.2 mmol) and 4-[1-methyl-4-(trifluoromethyl)imidazol-2-yl]benzaldehyde (258 mg, 1.01 mmol). The mixture solution was stirred for 16 hours at 60° C. then diluted with water (100 mL). The resulting mixture was extracted with ethyl acetate (3×100 mL). The combined organic layers were dried over anhydrous sodium sulfate. After filtration, the filtrate was concentrated under reduced pressure. The residue was purified by silica gel column chromatography, eluted with methanol in dichloromethane (0%-9%) to afford a crude product (112 mg). The crude product was purified by reverse phase chromatography with the following conditions: Column: Spherical C18, 20-40 um, 40 g; Mobile Phase A: Water (5 mM aq. NH₄HCO₃), Mobile Phase B: MeCN; Flow rate: 40 mL/min; Gradient (B %): 0% hold 5 min, 5%-45% within 10 min; 45%-70% within 20 min. 70%-95% within 2 min, 95% hold 5 min; Detector: UV 254 & 220 nm; RT: 23 min. The fractions were collected and concentrated under reduced pressure and then lyophilized overnight to give [2-(4-cyclopropyl-2-methoxy-3-pyridyl)-5H-pyrrolo[3,2-d]pyrimidin-7-yl]-[4-[1-methyl-4-(trifluoromethyl)imidazol-2-yl]phenyl]methanol (90 mg, 0.18 mmol, 17% yield) as an off-white solid. MS: m/z=521.20 [M +H]⁺. ¹H NMR (400 MHz, Methanol-d₄) δ 8.96 (s, 1H), 8.10 (d, J=5.6 Hz, 1H), 7.78 (s, 1H), 7.75-7.67 (m, 3H), 7.67-7.59 (m, 2H), 6.60 (d, J=5.6 Hz, 1H), 6.43 (s, 1H), 3.84 (s, 3H), 3.78 (s, 3H), 1.53-1.45 (m, 1H), 0.88-0.75 (m, 4H). ¹⁹F NMR (376 MHz, Methanol-d₄) δ −63.94.

### 1.4 2-(4-cyclopropyl-2-methoxy-3-pyridyl)-7-[[4-[1-methyl-4-(trifluoromethyl)imidazol-2-yl]phenyl]methyl]-5H-pyrrolo[3,2-d]pyrimidine

To a solution of [2-(4-cyclopropyl-2-methoxy-3-pyridyl)-5H-pyrrolo[3,2-d]pyrimidin-7-yl]-[4-[1-methyl-4-(trifluoromethyl)imidazol-2-yl]phenyl]methanol (0.125 g, 105.7 μmol) in chloroform (2 mL) were added trifluoroactic acid (1 mL) and triethylsilane (2 mL). The resulting solution was stirred at room temperature for 16 hours. The resulted reaction was concentrated under reduced pressure. The residue was purified directly by Prep-TLC (dichloromethane: methanol=10:1) to afford a crude product (77 mg). The crude product was further purified by reverse phase chromatography with the following conditions: Column: Spherical C18, 20-40 um, 40 g; Mobile Phase A: Water (5 mM aq. NH₄HCO₃), Mobile Phase B: MeCN; Flow rate: 40 mL/min; Gradient (B %): 0% hold 5 min, 5%-40% within 10 min; 40%-60% within 20 min, 60%-95% within 5 min, 95% hold 5 min; Detector: UV 254 & 220 nm; RT: 25 min. The fractions were collected, concentrated under reduced pressure and then lyophilized overnight to afford 2-(4-cyclopropyl-2-methoxy-3-pyridyl)-7-[[4-[1-methyl-4-(trifluoromethyl)imidazol-2-yl]phenyl]methyl]-5H-pyrrolo[3,2-d]pyrimidine (46 mg, 91.18 μmol, 86% yield) as an off-white solid. MS: m/z=505.20 [M+H]⁺. ¹H NMR (400 MHz, Methanol-d₄) δ 8.95 (s, 1H), 8.10 (d, J=5.6 Hz, 1H), 7.73 (s, 1H), 7.67 (s, 1H), 7.60-7.48 (m, 4H), 6.60 (d, J=5.6 Hz, 1H), 4.29 (s, 2H), 3.85 (s, 3H), 3.77 (s, 3H), 1.54-1.43 (m, 1H), 0.88-0.74 (m, 4H). ¹⁹F NMR (376 MHz, Methanol-d₄) δ −63.94.

### Example 62. 5-(4-cyclopropyl-2-methoxy-3-pyridyl-3-[[4-[1-methyl-4-(trifluoromethyl)imidazol-2-yl]phenyl]methyl]-1-pyrazolo[4,3-d]pyrimidine Compound 205

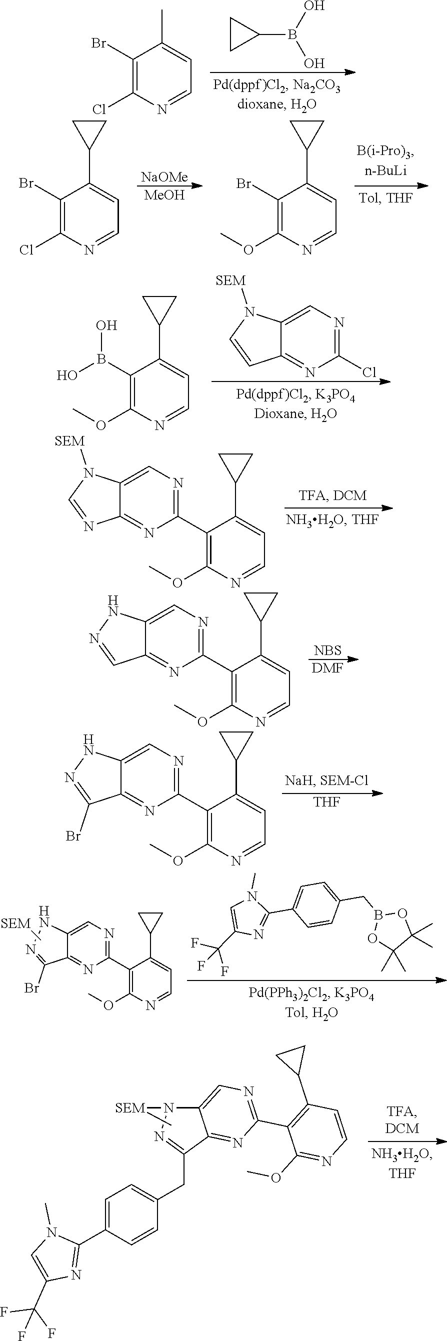

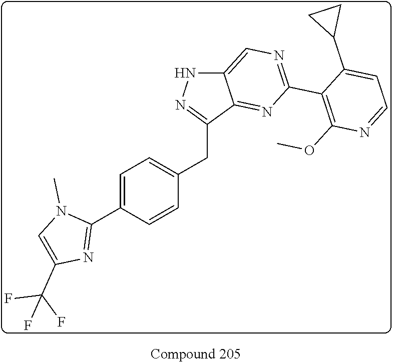 [SMILES: COc1nccc(C2CC2)c1-c1ncc2[nH]nc(Cc3ccc(-c4nc(C(F)(F)F)cn4C)cc3)c2n1]

### 1.1 3-bromo-2-chloro-4-cyclopropylpyridine

To a solution of 3-bromo-2-chloro-4-iodopyridine (2 g, 6.28 mmol) and cyclopropylboronic acid (540 mg, 6.28 mmol) in 1,4-dioxane (3.4 mL) and H₂O (0.80 mL) were added 1,1′-bis(diphenylphosphino)ferrocene-palladium(II)dichloride (513 mg, 628 μmol) and sodium carbonate (2.00 g, 18.85 mmol). The reaction mixture was stirred at 100° C. for 16 hours under nitrogen atmosphere then cool down to 25° C., and diluted by ethyl acetate (200 mL). The organic layer was washed with brine (2×200 mL), dried over anhydrous sodium sulfate. After filtration, the filtrate was concentrated under reduced pressure. The residue was purified by silica gel column chromatography, eluted with 15%-20% ethyl acetate in petroleum ether to afford 3-bromo-2-chloro-4-cyclopropylpyridine (865 mg, 3.72 mmol, 59% yield) as a white solid. MS: m/z=231.85, 233.85 [M+H]⁺. ¹H NMR (400 MHz, Chloroform-d) δ 8.18 (d, J=5.2 Hz, 1H), 6.68 (d, J=4.8 Hz, 1H), 2.34-2.27 (m, 1H), 1.27-1.12 (m, 2H), 0.85-0.77 (m, 2H).

### 1.2 3-bromo-4-cyclopropyl-2-methoxypyridine

To a solution of 3-bromo-2-chloro-4-cyclopropylpyridine (800 mg, 3.44 mmol) in methanol (8 mL) was added sodium methanolate (3.72 g, 10.32 mmol, 15% in methanol) under nitrogen atmosphere. The reaction mixture was stirred at 70° C. for 16 hours then cool down to 25° C. The resulted reaction was diluted by ethyl acetate (200 mL). The organic layer was washed with ammonium chloride aqueous solution (2×200 mL), dried over anhydrous sodium sulfate. After filtration, the filtrate was concentrated under reduced pressure. The residue was purified by silica gel column chromatography, eluted with 20%-25% ethyl acetate in petroleum ether to afford 3-bromo-4-cyclopropyl-2-methoxypyridine (700 mg, 3.07 mmol, 89% yield) as a light-yellow oil. MS: m/z=227.95, 229.95 [M+H]⁺. ¹H NMR (400 MHz, Chloroform-<) S 7.95 (d, J=5.2 Hz, 1H), 6.35 (d, J=5.6 Hz, 1H), 4.04 (s, 3H), 2.35-2.28 (m, 1H), 1.27-1.09 (m, 2H), 0.84-0.76 (m, 2H).

### 1.3 (4-cyclopropyl-2-methoxy-3-pyridyl) boronic Acid

To a solution of 3-bromo-4-cyclopropyl-2-methoxypyridine (610 mg, 2.67 mmol) and triisopropyl borate (654 mg, 3.48 mmol, 802 μL) in dry toluene (10 mL) and THF (2.5 mL) was added n-butyllithium (0.52 mL, 1.3 mmol, 2.5 M in hexane) at −78° C. under nitrogen atmosphere. The reaction mixture was stirred at this temperature for 10 min. then warmed up to room temperature and quenched with 1 M Hydrogen chloride aqueous solution (0.425 mL).

After concentration under vacuum the crude product was directly purified by RP-Flash chromatography with the following conditions: Column: C18 spherical, 20-35 μm, 100A, 40 g; Mobile Phase A: 5 mM aq. HCl, Mobile Phase B: MeCN; Flow rate: 35 mL/min; Gradient: 5% B to 5% B in 10 min, 5% B to 25% B in 25 min; 25% B to 25% B in 5 min; 25% B to 95% B in 5 min; Detector: UV 254 & 220 nm; RT: 20 min. The fractions were collected, concentrated under reduced pressure and then lyophilized overnight to give (4-cyclopropyl-2-methoxy-3-pyridyl) boronic acid (363 mg, 1.88 mmol, 70% yield) as an off-white solid. MS: m/z=194.05 [M+H]⁺. ¹H NMR (400 MHz, DMSO-d₆) δ 7.92 (d, J=5.6 Hz, 1H), 6.39 (d, J=5.6 Hz, 1H), 3.80 (s, 3H), 1.84-1.78 (m, 1H), 1.04-0.94 (m, 2H), 0.77-0.73 (m, 2H).

### 1.4 2-[[5-(4-cyclopropyl-2-methoxy-3-pyridyl)pyrazolo[4,3-d]pyrimidin-1-yl]methoxy]ethyl-trimethyl-silane

To a solution of (4-cyclopropyl-2-methoxy-3-pyridyl)boronic acid (360 mg, 1.87 mmol) and 2-[(5-chloropyrazolo[4,3-d]pyrimidin-1-yl)methoxy]ethyl-trimethyl-silane (531 mg, 1.87 mmol) in 1,4-dioxane (10 mL) and H₂O (2 mL) were added 1,1′-Bis(diphenylphosphino)ferrocene-palladium(II)dichloride (152 mg, 0.187 mmol) and potassium phosphate (1.19 g, 5.60 mmol). The reaction solution was stirred at 100° C. for 16 hours under nitrogen atmosphere then cooled down to 25° C., and diluted by ethyl acetate (100 mL). The organic layer was washed with brine (2×100 mL), dried over anhydrous sodium sulfate. After filtration, the filtrate was concentrated under reduced pressure. The residue was purified by silica gel column chromatography, eluted with 1%-5% methanol in DCM to give 2-[[5-(4-cyclopropyl-2-methoxy-3-pyridyl)pyrazolo[4,3-d]pyrimidin-1-yl]methoxy]ethyl-trimethyl-silane (430 mg, 1.08 mmol, 57% yield) as a light yellow oil. MS: m/z=398.15 [M+H]⁺.

### 1.5 5-(4-cyclopropyl-2-methoxy-3-pyridyl)-1H-pyrazolo[4,3-d]pyrimidine

To a stirred solution of 2-[[5-(4-cyclopropyl-2-methoxy-3-pyridyl)pyrazolo[4,3-d]pyrimidin-1-yl]methoxy]ethyl-trimethyl-silane (430 mg, 1.08 mmol) in DCM (10 mL) was added trifluoroacetic acid (10 mL) and then stirred for 2 hours at 25° C. The reaction was concentrated under reduced pressure to give a residue. Ammonium hydroxide (10 mL, 28% aqueous solution) and THF (10 mL) were added to the residue. The resulting mixture was stirred for 30 min at 25° C. then dissolved with ethyl acetate (100 mL), washed by brine (3×100 mL). The organic layer was dried over anhydrous sodium sulfate. After filtration, the filtrate was concentrated under reduced pressure. The residue was purified by silica gel column chromatography, eluted with 5%-10% methanol in dichloromethane to afford 5-(4-cyclopropyl-2-methoxy-3-pyridyl)-1H-pyrazolo[4,3-d]pyrimidine (263 mg, 983.97 μmol, 90% yield) as a white solid. MS: m/z=268.10 [M+H]⁺. ¹H NMR (400 MHz, Methanol-d₄) δ 9.41 (s, 1H), 8.38 (s, 1H), 8.12 (d, J=5.6 Hz, 1H), 6.62 (d, J=5.6 Hz, 1H), 3.84 (s, 3H), 1.54-1.48 (m, 1H), 0.91-0.72 (m, 4H).

### 1.6 3-bromo-5-(4-cyclopropyl-2-methoxy-3-pyridyl)-1H-pyrazolo[4,3-d]yrimidine

To a solution of 5-(4-cyclopropyl-2-methoxy-3-pyridyl)-1H-pyrazolo[4,3-d]pyrimidine (263 mg, 983.97 μmol) in N,N-dimethylformamide (889 μL) was added 1-bromopyrrolidine-2,5-dione (263 mg, 1.48 mmol) at 25° C. under nitrogen atmosphere. The reaction mixture was stirred at this temperature for 3 hours and then dissolved by ethyl acetate (100 mL). The resulting mixture was washed by brine (3×100 mL). The organic layer was dried over anhydrous sodium sulfate. After filtration, the filtrate was concentrated under reduced pressure. The residue was purified by silica gel column chromatography, eluted with 40%-45% ethyl acetate in petroleum ether to afford 3-bromo-5-(4-cyclopropyl-2-methoxy-3-pyridyl)-1H-pyrazolo[4,3-d]pyrimidine (250 mg, 722.16 μmol, 73% yield) as a brown solid. MS: m/z=345.90, 347.90 [M+H]⁺. ¹H NMR (400 MHz, Chloroform-d) δ 12.13 (br., 1H), 9.34 (s, 1H), 8.15 (d, J=5.6 Hz, 1H), 6.50 (d, J=5.6 Hz, 1H), 3.93 (s, 3H), 1.61-1.34 (m, 1H), 0.98-0.71 (m, 4H).

### 1.7 3-bromo-5-(4-cyclopropyl-2-methoxypyridin-3-yl)-1-((2-(trimethylsilyl)ethoxy)methyl)-1H-pyrazolo[4,3-d]pyrimidine & 3-bromo-5-(4-cyclopropyl-2-methoxypyridin-3-yl)-2-((2-(trimethylsilylethoxy)methyl)-2H-pyrazolo[4,3-d]pyrimidine

To a solution of 3-bromo-5-(4-cyclopropyl-2-methoxy-3-pyridyl)-1H-pyrazolo[4,3-d]pyrimidine (248 mg, 716.39 μmol) in THF (10 mL) was added sodium (34 mg, 860 μmol, 60% in oil) at 0° C. under nitrogen atmosphere, and the reaction mixture was stirred at 0° C. for 1 hour. Then 2-(chloromethoxy)ethyl-trimethyl-silane (119 mg, 716 μmol, 127 μL) was added to the reaction mixture and stirred at 25° C. for 2 hours. The reaction was quenched by the addition of saturated ammonium chloride aqueous solution (100 mL). The resulted mixture was extracted with ethyl acetate (3×100 mL). The combined organic layers were washed with brine (3×100 mL), dried over anhydrous sodium sulfate. After filtration, the filtrate was concentrated under reduced pressure. The residue was purified by silica gel column chromatography, eluted with 1%-5% methanol in dichloromethane to give 3-bromo-5-(4-cyclopropyl-2-methoxypyridin-3-yl)-1-((2-(trimethylsilyl)ethoxy)methyl)-1H-pyrazolo[4,3-d]pyrimidine & 3-bromo-5-(4-cyclopropyl-2-methoxypyridin-3-yl)-2-((2-(trimethylsilyl)ethoxy)methyl)-2H-pyrazolo[4,3-d]pyrimidine (260 mg, 545.71 μmol, 76% yield) as a light yellow oil. MS: m/z=475.95, 477.95 [M+H]⁺.

### 1.8 5-(4-cyclopropyl-2-methoxypyridin-3-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-cyclopropyl-2-methoxypyridin-3-yl)-3-(4-(1-methyl-4-(trifluoromethyl)-1H-imidazol-2-yl)benzyl)-24(2-(trimethylsilylethoxy)methyl)-2H-pyrazolo[4,3-d]pyrimidine

To a solution of 3-bromo-5-(4-cyclopropyl-2-methoxypyridin-3-yl)-1-((2-(trimethylsilyl)ethoxy)methyl)-1H-pyrazolo[4,3-d]pyrimidine & 3-bromo-5-(4-cyclopropyl-2-methoxypyridin-3-yl)-2-((2-(trimethylsilyl)ethoxy)methyl)-2H-pyrazolo[4,3-d]pyrimidine (250 mg, 524.72 μmol) and 1-methyl-2-[4-[(4,4,5,5-tetramethyl-1,3,2-dioxaborolan-2-yl)methyl]phenyl]-4-(trifluoromethyl)imidazole (192 mg, 525 μmol) were added dichlorobis(triphenylphosphine)Palladium (37 mg, 52.5 μmol) and potassium phosphate (223 mg, 1.05 mmol) at 25° C. The resulting mixture was stirred for 16 hours at 90° C. then cooled down to room temperature. The mixture was filtered through a celite pad. After filtration, the filtrate was concentrated under reduced pressure. The residue was purified by silica gel column chromatography, eluted with 45%-50% ethyl acetate in petroleum ether to afford 5-(4-cyclopropyl-2-methoxypyridin-3-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-cyclopropyl-2-methoxypyridin-3-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (45 mg, 70.78 μmol, 13% yield) as a brown oil. MS: m/z=636.25 [M+H]⁺.

### 1.9 5-(4-cyclopropyl-2-methoxy-3-pyridyl)-3-[[4-[1-methyl-4-(trifluoromethyl)imidazol-2-yl]phenyl]methyl]-1H-pyrazolo[4,3-d]pyrimidine

To a stirred solution of 5-(4-cyclopropyl-2-methoxypyridin-3-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-cyclopropyl-2-methoxypyridin-3-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (45 mg, 70.78 μmol) in DCM (1 mL) was added trifluoroacetic acid (1 mL) and then stirred for 2 hours at 25° C. The resulting mixture was concentrated under reduced pressure. Then ammonium hydroxide (1 mL, 28% aqueous solution) and THF (1 mL) were added in the above residue. The resulting mixture was stirred for 30 min at 25° C. then concentrated under reduced pressure. The residue was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 20 mL/min; Gradient: 5% B to 5% B in 5 min, 5% B to 63% B in 10 min, 63% B to 63% B in 2 min, 63% B to 95% B in 5 min; Detector: UV 254 & 210 nm; RT: 17 min. The collected fractions were combined, concentrated and then lyophilized to give 5-(4-cyclopropyl-2-methoxy-3-pyridyl)-3-[[4-[1-methyl-4-(trifluoromethyl)imidazol-2-yl]phenyl]methyl]-1H-pyrazolo[4,3-d]pyrimidine (11.70 mg, 22.30 μmol, 31% yield) as a white solid. MS: m/z=506.40 [M+H]⁺. ¹H NMR (400 MHz, Chloroform-d) δ 13.69 (br., 1H), 9.10 (s, 1H), 8.09 (d, J=5.6 Hz, 1H), 7.65 (d, J=8.0 Hz, 2H), 7.57 (d, J=8.0 Hz, 2H), 7.40 (s, 1H), 6.43 (d, J=5.6 Hz, 1H), 4.54 (s, 2H), 3.84 (s, 3H), 3.80 (s, 3H), 1.55-1.44 (m, 1H), 0.80-0.70 (m, 4H). ¹⁹F NMR (376 MHz, Chloroform-d) δ −62.16.

### Example 63. 2-(2-cyclopropyl-4-methoxy-3-pyridyl-7-[[4-[1-methyl-4-(trifluoromethyl)imidazol-2-yl]phenyl]methyl]-5H-pyrrolo[3,2-d]pyrimidine Compound

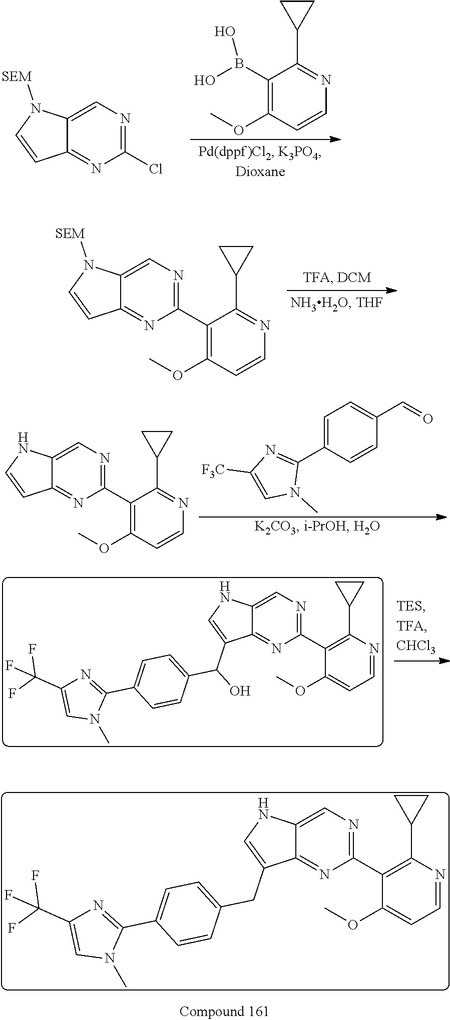

### 1.1 2-[[2-(2-cyclopropyl-4-methoxy-3-pyridyl)pyrrolo[3,2-d]pyrimidin-5-yl]methoxy]ethyl-trimethyl-silane

To a solution of 2-[(2-chloropyrrolo[3,2-d]pyrimidin-5-yl)methoxy]ethyl-trimethyl-silane (779 mg, 2.75 mmol) in 1,4-dioxane (20 mL) were added (2-cyclopropyl-4-methoxy-3-pyridyl)boronic acid (530 mg, 2.75 mmol), [1,1′-Bis(diphenylphosphino)ferrocene]dichloropalladium(II) (224 mg, 275 mot) and potassium phosphate tribasic (1.75 g, 8.24 mmol) at 15° C. under nitrogen atmosphere. The resulted reaction was stirred at 100° C. for 16 hours under nitrogen atmosphere. The resulted solution was detected by LCMS, could find 7% desired product. The resulted solution was filtrated. The filtrate was concentrated under reduced pressure. The residue was purified by silica gel column chromatography, eluted with 20%-60% ethyl acetate in petroleum ether to give 2-[[2-(2-cyclopropyl-4-methoxy-3-pyridyl)pyrrolo[3,2-d]pyrimidin-5-yl]methoxy]ethyl-trimethyl-silane (140 mg, 353.04 μmol) as a white solid. MS: m/z=397.15 [M+H]⁺.

### 1.2 2-(2-cyclopropyl-4-methoxy-3-pyridyl)-5H-pyrrolo[3,2-d]pyrimidine

To a solution of 2,2,2-trifluoroacetic acid (2 mL) in dichloromethane (2 mL) was added 2-[[2-(2-cyclopropyl-4-methoxy-3-pyridyl)pyrrolo[3,2-d]pyrimidin-5-yl]methoxy]ethyl-trimethyl-silane (140 mg, 353.04 μmol) at 15° C. The resulted reaction was stirred at 15° C. for 2 hours. The resulted solution was detected by LCMS, major was desired product. The resulted solution was concentrated under reduced pressure. The residue was added tetrahydrofuran (2.5 mL) and ammonia solution (25% in H₂O, 2.5 mL). The resulted solution was stirred at 15° C. for 10 min. The resulted solution was detected by LCMS, major was desired product. The resulted solution was concentrated under reduced pressure. The residue was purified by silica gel column chromatography, eluted with 0%-15% methanol in dichloromethane to give 130 mg crude product. The crude product was further purified by Prep-TLC (dichloromethane:methanol=8: 1) to afford 2-(2-cyclopropyl-4-methoxy-3-pyridyl)-5H-pyrrolo[3,2-d]pyrimidine (52 mg, 195.27 μmol, 55% yield) as a yellow solid. MS: m/z=267.00 [M+H]⁺. ¹H NMR (400 MHz, Chloroform-d) δ 9.18 (s, 1H), 9.05 (s, 1H), 8.42 (d, J=5.6 Hz, 1H), 7.66 (d, J=3.2 Hz, 1H), 6.84 (d, J=3.2 Hz, 1H), 6.74 (d, J=6.0 Hz, 1H), 3.77 (s, 3H), 1.61-1.54 (m, 1H), 1.23-1.10 (m, 2H), 0.83-0.74 (m, 2H).

### 1.3 [2-(2-cyclopropyl-4-methoxy-3-pyridyl)-5H-pyrrolo[3,2-d]pyrimidin-7-yl]-[4-[1-methyl-4-(trifluoromethyl)imidazol-2-yl]phenyl]methanol

To a solution of 2-(2-cyclopropyl-4-methoxy-3-pyridyl)-5H-pyrrolo[3,2-d]pyrimidine (27 mg, 101.39 μmol) in water (0.5 mL) and isopropyl alcohol (0.5 mL) was added 4-[1-methyl-4-(trifluoromethyl)imidazol-2-yl]benzaldehyde (26 mg, 101.4 μmol) at 15° C., then was stirred at 60° C. for 16 hours. The resulted solution was detected by LCMS, could find 27% desired product overlap with by-product. The resulted solution was diluted by ethyl acetate (20 mL), then was washed with saturated sodium chloride aqueous solution (10 mL×3). The organic layer was dried over anhydrous sodium sulfate. After filtration, the filtrate was concentrated under reduced pressure. The residue was purified by Prep-TLC (dichloromethane:methanol=11:1) to give 11 mg crude product. The crude product was purified by RP-Flash chromatography with the following conditions: Column: C18 spherical, 20-35 um, 100A, 20 g; Mobile Phase A: 10 mM aq. ammonium bicarbonate, Mobile Phase B: acetonitrile; Flow rate: 20 mL/min; Gradient: 5% B to 5% B in 5 min, 5% B to 20% B in 5 min, 20% B to 40% B in 20 min, 40% B to 95% B in 5 min; Detector: UV 254 & 220 nm; RT: 20 min to give [2-(2-cyclopropyl-4-methoxy-3-pyridyl)-5H-pyrrolo[3,2-d]pyrimidin-7-yl]-[4-[1-methyl-4-(trifluoromethyl)imidazol-2-yl]phenyl]methanol (2.5 mg, 4.80 μmol, 4% yield) as a white solid. MS: m/z=521.30 [M+H]⁺. ¹H NMR (400 MHz, Chloroform-d) δ 9.79 (br., 1H), 9.00 (s, 1H), 8.48 (d, J=6.0 Hz, 1H), 7.70-7.60 (m, 4H), 7.37 (s, 1H), 7.14 (s, 1H), 6.88 (d, J=5.2 Hz, 1H), 6.42 (s, 1H), 4.33 (br., 1H), 3.86 (s, 3H), 3.80 (s, 3H), 1.80-1.70 (m, 1H), 1.36-1.27 (s, 2H), 0.81-0.79 (m, 2H). ¹⁹F NMR (376 MHz, Chloroform-d) δ −62.56.

### 1.4 2-(2-cyclopropyl-4-methoxy-3-pyridyl)-7-[[4-[1-methyl-4-(trifluoromethyl)imidazol-2-yl]phenyl]methyl]-5H-pyrrolo[3,2-d]pyrimidine

To a solution of [2-(2-cyclopropyl-4-methoxy-3-pyridyl)-5H-pyrrolo[3,2-d]pyrimidin-7-yl]-[⁴-[1-methyl-4-(trifluoromethyl)imidazol-2-yl]phenyl]methanol (17 mg, 32.66 μmol) in chloroform (1 mL) was added 2,2,2-trifluoroacetic acid (0.5 mL) and triethylsilane (1 mL) at 15° C., then was stirred at 15° C. for 3 hours. The resulted solution was detected by LCMS, major desired product. The resulted solution was concentrated under reduced pressure. The residue was co-evaporated with toluene (three times, 2.0 mL each). The resulted residue was purified by RP-Flash chromatography with the following conditions: Column: C18 spherical, 20-35 um, 100A, 20 g; Mobile Phase A: 10 mM aq. ammonium bicarbonate, Mobile Phase B: acetonitrile; Flow rate: 20 mL/min; Gradient: 5% B to 5% B in 5 min, 5% B to 25% B in 5 min, 25% B to 45% B in 20 min, 45% B to 95% B in 5 min; Detector: UV 254 & 220 nm; RT: 25 min to give 2-(2-cyclopropyl-4-methoxy-3-pyridyl)-7-[[4-[1-methyl-4-(trifluoromethyl)imidazol-2-yl]phenyl]methyl]-5H-pyrrolo[3,2-d]pyrimidine (4.9 mg, 9.71 μmol, 29% yield) as a white solid. MS: m/z=505.30 [M+H]⁺. ¹H NMR (400 MHz, Chloroform-d) δ 9.80 (br., 1H), 8.97 (s, 1H), 8.46 (d, J=6.0 Hz, 1H), 7.54 (d, J=8.0 Hz, 2H), 7.44 (d, J=8.0 Hz, 2H), 7.35 (s, 1H), 7.22 (s, 1H), 6.82 (d, J=5.6 Hz, 1H), 4.29 (s, 2H), 3.83 (s, 3H), 3.78 (s, 3H), 1.65-1.58 (m, 1H), 1.28-1.22 (s, 2H), 0.80-0.78 (s, 2H). ¹⁹F NMR (376 MHz, Chloroform-d) δ −62.55.

### Example 64. [2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol Compound 168

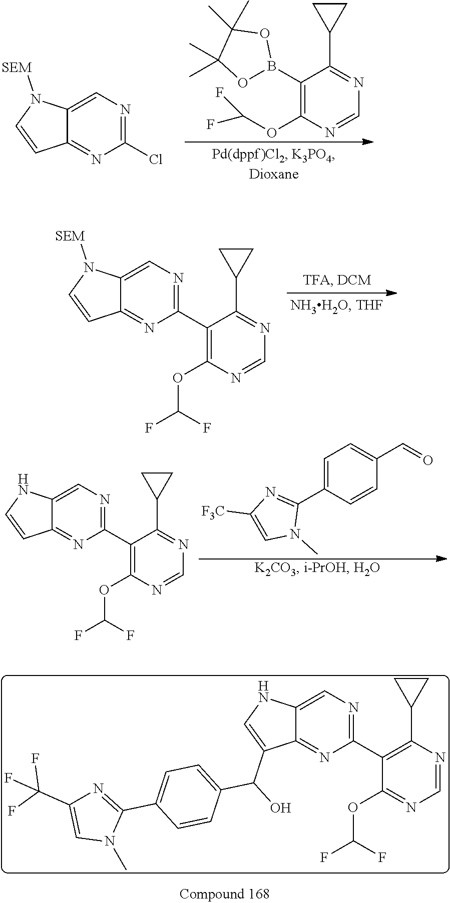

### 1.1 2-[[2-[4-cyclopropyl-6-(difluoromethoxy)pyrimidin-5-yl]pyrrolo[3,2-d]pyrimidin-5-yl]methoxy]ethyl-trimethyl-silane

To a solution of 2-[(2-chloropyrrolo[3,2-d]pyrimidin-5-yl)methoxy]ethyl-trimethyl-silane (900 mg, 3.17 mmol) and 4-cyclopropyl-6-(difluoromethoxy)-5-(4,4,5,5-tetramethyl-1,3,2-dioxaborolan-2-yl)pyrimidine (990 mg, 3.17 mmol) in 1,4-dioxane (2 mL) were added 1,1′-bis(diphenylphosphino)ferrocene palladium(II)dichloride (259 mg, 317 μmol) and potassium phosphate (2.02 g, 9.51 mmol) at 25° C. under nitrogen atmosphere. The mixture was heated to 100° C., and stirred for 16 hours under nitrogen atmosphere. The resulted mixture was allowed to cool down to 25° C. Then the mixture was diluted with ethyl acetate (100 mL) and washed with brine (3×40 mL), dried over anhydrous sodium sulfate. After filtration, the filtrate was concentrated under reduced pressure. The residue was purified by silica gel column chromatography, eluted with 20%-25% ethyl acetate in petroleum ether to give 2-[[2-[4-cyclopropyl-6-(difluoromethoxy)pyrimidin-5-yl]pyrrolo[3,2-d]pyrimidin-5-yl]methoxy]ethyl-trimethyl-silane (150 mg, 346.00 μmol, 11% yield) as a yellow solid. MS: m/z=434.30 [M+H]. ¹H NMR (400 MHz, Chloroform-d) δ 9.15 (s, 1H), 8.69 (s, 1H), 7.68 (d, J=3.2 Hz, 1H), 7.50 (t, J=73.2 Hz, 1H), 6.82 (d, J=3.2 Hz, 11H), 5.63 (s, 2H), 3.64-3.55 (m, 2H), 1.85-1.79 (m, 1H), 1.35-1.25 (m, 2H), 1.01-0.95 (m, 4H), −0.00 (s, 9H).

### 1.2 2-[4-cyclopropyl-6-(difluoromethoxy)pyrimidin-5-yl]-5H-pyrrolo[3,2-d]pyrimidine

To a solution of 2-[[2-[4-cyclopropyl-6-(difluoromethoxy)pyrimidin-5-yl]pyrrolo[3,2-d]pyrimidin-5-yl]methoxy]ethyl-trimethyl-silane (150 mg, 346 μmol) in dichloromethane (3 mL) was added trifluoroacetic acid (4.44 g, 38.94 mmol, 3 mL) at 0° C. under nitrogen atmosphere. The reaction mixture was stirred at 25° C. for 2 hours. The reaction solution was concentrated under reduce pressure. Then the residue was added ammonium hydroxide (28% NH₃ in H₂O, 3 mL) and tetrahydrofuran (3 mL). The mixture was stirred at 25° C. for 30 minutes. The reaction solution was concentrated under reduce pressure. The residue was purified by silica gel column chromatography, eluted with 60%-70% ethyl acetate in petroleum ether to give 2-[4-cyclopropyl-6-(difluoromethoxy)pyrimidin-5-yl]-5H-pyrrolo[3,2-d]pyrimidine (95 mg, 313.26 μmol, 90% yield) as a yellow solid. MS: m/z=304.05 [M+H]⁺. ¹H NMR (400 MHz, Methanol-d₄) δ 9.06 (s, 1H), 8.72 (s, 1H), 8.00 (d, J=3.2 Hz, 1H), 7.64 (t, J=73.2 Hz, 1H), 6.77 (d, J=3.2 Hz, 1H), 1.74-1.68 (m, 1H), 1.27-1.23 (m, 2H), 1.02-0.99 (m, 2H).

### 1.3 [2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol

To a solution of 2-[4-cyclopropyl-6-(difluoromethoxy)pyrimidin-5-yl]-5H-pyrrolo[3,2-d]pyrimidine (90 mg, 297 μmol) and 4-[1-methyl-4-(trifluoromethyl)imidazol-2-yl]benzaldehyde (75 mg, 296 μmol) in isopropanol (1 mL) and water (1 mL) was added potassium carbonate (49 mg, 356 μmol) and at 25° C. under nitrogen atmosphere. The mixture was heated to 60° C., and stirred for 8 hours. The resulted mixture was allowed to cool down to 25° C. The resulted mixture was diluted with ethyl acetate (200 mL) and washed with brine (3×70 mL), dried over anhydrous sodium sulfate. After filtration, the filtrate was concentrated under reduced pressure. The residue was purified by silica gel column chromatography, eluted with 5%-7% methyl alcohol in dichloromethane to give to afford a crude product. The crude product was further purified by RP-Flash chromatography with the following conditions: Column: Column: C18 spherical Column, 20-35 um, 100A, 40 g; Mobile Phase A: Water (5 mM aq. NH₄HCO₃), Mobile Phase B: MeCN; Flow rate: 40 ml/min; Gradient: 5% B to 5% B in 5 min, 5% B to 42% B in 10 min, 42% B to 42% B in 4 min. 42% B to 95% B in 10 min; Detector: UV 254 & 210 nm; RT: 18 min. The fractions of first eluting peak (RT: 18 min) were collected and concentrated under reduced pressure and then lyophilized overnight to afford [2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol (24 mg, 43.05 μmol, 15% yield) as a white solid. MS: m/z=558.25 [M+H]⁺. ¹H NMR (400 MHz, Chloroform-d) δ 10.73 (s, 1H), 8.92 (s, 1H), 8.69 (s, 1H), 7.68 (d, J=8.0 Hz, 2H), 7.61 (d, J=8.0 Hz, 2H), 7.53 (t, J=73.2 Hz, 1H), 7.37 (s, 1H), 7.19 (s, 1H), 6.38 (s, 1H), 3.78 (s, 3H), 1.93-1.88 (m, 1H), 1.30-1.28 (m, 2H), 1.00-0.97 (m, 2H). ¹⁹F NMR (376 MHz, Chloroform-d) δ −62.18, −89.38

### Example 65. 2-[4-cyclopropyl-6-(difluoromethoxy)pyrimidin-5-yl]-7-[[4-[1-methyl-4-(trifluoromethyl)imidazol-2-yl]phenyl]methyl]-5H-pyrrolo[3,2-d]pyrimidine Compound 138

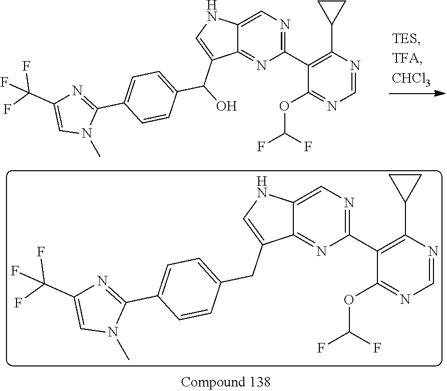 [SMILES: Cn1cc(C(F)(F)F)nc1-c1ccc(C(O)c2c[nH]c3cnc(-c4c(OC(F)F)ncnc4C4CC4)nc23)cc1 ; Cn1cc(C(F)(F)F)nc1-c1ccc(Cc2c[nH]c3cnc(-c4c(OC(F)F)ncnc4C4CC4)nc23)cc1]

### 1.1 2-[4-cyclopropyl-6-(difluoromethoxy)pyrimidin-5-yl]-7-[[4-[1-methyl-4-(trifluoromethyl)imidazol-2-yl]phenyl]methyl]-5H-pyrrolo[3,2-d]pyrimidine

To a solution of [2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol (20 mg, 36 μmol) in chloroform (2 mL) were added triethylsilane (1.46 g, 12.52 mmol, 2 mL) and trifluoroacetic acid (1.48 g, 12.98 mmol, 1 mL) at 0° C. The reaction mixture was stirred at 25° C. for 2 h. The reaction solution was concentrated under reduce pressure. The residue was purified by RP-Flash chromatography with the following conditions: Column: Column: C18 spherical Column, 20-35 um, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 65% B in 10 min, 65% B to 65% B in 3 min, 65% B to 95% B in 5 min; Detector: UV 254 & 210 nm; RT: 18 min. The fractions were collected, concentrated under reduced pressure and then lyophilized overnight to afford 2-[4-cyclopropyl-6-(difluoromethoxy)pyrimidin-5-yl]-7-[[4-[1-methyl-4-(trifluoromethyl)imidazol-2-yl]phenyl]methyl]-5H-pyrrolo[3,2-d]pyrimidine (7.0 mg, 12.93 μmol, 36% yield) as a white solid. MS: m/z=542.15 [M+H]⁺. ¹H NMR (400 MHz, Chloroform-d) δ 10.37 (s, 1H), 8.98 (s, 1H), 8.68 (s, 1H), 7.54 (d. J=8.0 Hz, 2H), 7.51 (t, J=73.2 Hz, 1H), 7.45 (d, J=8.0 Hz, 2H), 7.32 (s, 1H), 7.27 (s, 1H), 4.28 (s, 2H), 3.77 (s, 3H), 1.92-1.86 (m, 1H), 1.30-1.27 (m, 2H), 0.99-0.91 (m, 2H). F NMR (376 MHz, Chloroform-d) δ −62.45, −89.39.

### Example 66. 2-(4-cyclopropyl-6-methoxy-pyrimidin-5-yl)-4-methyl-7-[[4-[1-methyl-4-(trifluoromethyl)imidazol-2-yl]phenyl]methyl]-5H-pyrrolo[3,2-d]pyrimidine Compound 148

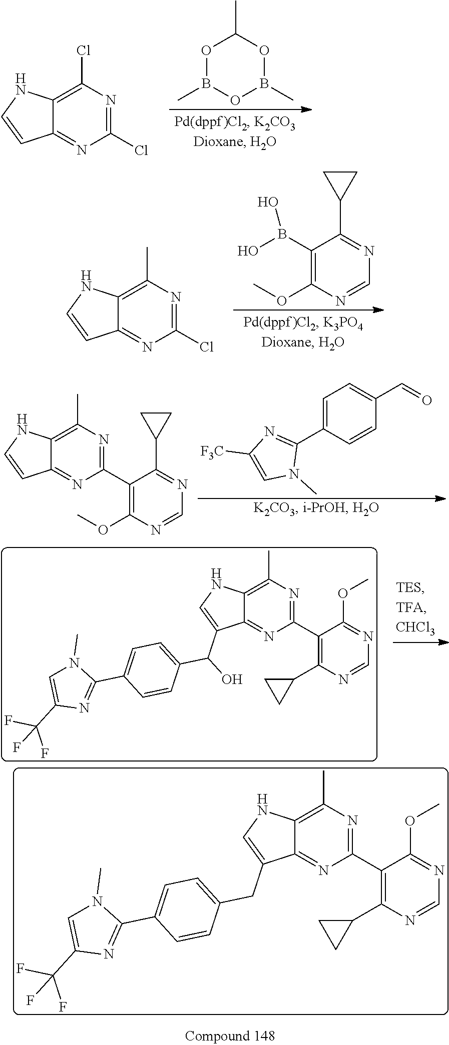

### 1.1 2-chloro-4-methyl-5H-pyrrolo[3,2-d]pyrimidine

To a solution of 2,4-dichloro-5H-pyrrolo[3,2-d]pyrimidine (2 g, 10.64 mmol) and 2,4,6-trimethyl-1,3,5,2,4,6-trioxatriborinane (1.34 g, 10.64 mmol) in water (7 mL) and dioxane (21 mL) were added 1,1′-Bis(diphenylphosphino)ferrocene-palladium(II)dichloride (868.70 mg, 1.06 mmol) and potassium carbonate (2.94 g, 21.28 mmol) at 25° C. under nitrogen atmosphere. The reaction mixture was stirred at 100° C. for 16 hours then cooled down to 25 TC. The resulting solution was quenched with 30 ml of water and extracted with ethyl acetate (3×50 ml). The combined organic layers were dried over anhydrous sodium sulfate and filtered. The filtrate was concentrated under reduced pressure. The residue was purified by silica gel column chromatography, eluted with 1%-7% methanol in dichloromethane to give 2-chloro-4-methyl-5H-pyrrolo[3,2-d]pyrimidine (1.75 g, 10.44 mmol, 98% yield) as a brown solid. MS: m/z =168.05, 170.05 [M+H]⁺. ¹H NMR (400 MH-z. Chloroform-d) δ 12.22 (s, 1H), 7.96 (d, J=3.2 Hz, 1H), 6.56 (d, J=2.8 Hz, 1H), 2.67 (s, 3H).

### 1.2 2-(4-cyclopropyl-6-methoxy-pyrimidin-5-yl)-4-methyl-5H-pyrrolo[3,2-d]pyrimidine

To a solution of 2-chloro-4-methyl-5H-pyrrolo[3,2-d]pyrimidine (1 g, 5.97 mmol) and (4-cyclopropyl-6-methoxy-pyrimidin-5-yl)boronic acid (1.16 g, 5.97 mmol) in 1,4-dioxane (10 mL) and water (2 mL) were added 1,1′-Bis(diphenylphosphino)ferrocene-palladium(II)dichloride (488 mg, 597 μmol) and potassium phosphate (3.80 g, 17.90 mmol) at 25° C. under nitrogen atmosphere. The reaction mixture was stirred at 100° C. for 16 hours then cooled down to room temperature and the solid was filtered out. The filtrate was concentrated under reduced pressure. The residue was purified by silica gel column chromatography, eluted with 1%-5% methanol in dichloromethane to give 2-(4-cyclopropyl-6-methoxy-pyrimidin-5-yl)-4-methyl-5H-pyrrolo[3,2-d]pyrimidine (150 mg, 533.22 μmol, 9% yield) as a yellow solid. MS: m/z=282.20 [M+H]⁺. ¹H NMR (400 MHz, Chloroform-d) δ 12.06 (s, 1H), 8.65 (s, 1H), 7.92-7.61 (m, 1H), 6.63-6.60 (m, 1H), 3.79 (s, 3H), 2.72 (s, 3H), 1.57-1.51 (m, 1H), 1.04-1.00 (m, 2H), 0.86-0.84 (m, 2H).

### 1.3 [2-(4-cyclopropyl-6-methoxy-pyrimidin-5-yl)-4-methyl-5H-pyrrolo[3,2-d]pyrimidin-7-yl]-[4-[1-methyl-4-(trifluoromethyl)imidazol-2-yl]phenyl]methanol

To a solution of 2-(4-cyclopropyl-6-methoxy-pyrimidin-5-yl)-4-methyl-5H-pyrrolo[3,2-d]pyrimidine (150 mg, 533.22 μmol) and 4-[1-methyl-4-(trifluoromethyl)imidazol-2-yl]benzaldehyde (136 mg, 533.22 μmol) in isopropyl alcohol (3 mL) were added potassium carbonate (88 mg, 640 μmol) and water (3 mL) at 25° C. The reaction mixture was stirred at 60° C. for 6 hours then cooled down to 25° C. The resulting solution was diluted with 20 ml of water, then extracted with ethyl acetate (3×20 ml). The combined organic layers were dried over anhydrous sodium sulfate and filtered. The filtrate was concentrated under reduced pressure. The residue was purified by silica gel column chromatography, eluted with 5%-10% methanol in dichloromethane to give a crude product. The crude product was purified by RP-Flash with the following conditions: Column: C18 spherical Column, 20-35 μm, 100A, 20 g; Mobile Phase A: Water (5 mM aq. NH₄HCO₃), Mobile Phase B: MeCN; Flow rate:25 mL/min; Gradient: 5% B to 5% B in 3 min, 5% B to 51% B in 20 min; 51% B to 51% B in 3 min, 51% B to 95% B in 15 min; Detector: UV 254 & 210 nm; RT: 24 min. The collected fractions were combined, concentrated and then lyophilized overnight to give [2-(4-cyclopropyl-6-methoxy-pyrimidin-5-yl)-4-methyl-5H-pyrrolo[3,2-d]pyrimidin-7-yl]-[4-[1-methyl-4-(trifluoromethyl)imidazol-2-yl]phenyl]methanol (34 mg, 63.49 μmol, 12% yield) as an off-white solid. MS: m/z=536.35 [M+H]⁺. ¹H NMR (400 MHz, Chloroform-d) δ 8.64 (s, 1H), 7.73-7.70 (m, 4H), 7.65-7.63 (m, 2H), 6.41 (s, 1H), 3.92 (s, 3H), 3.79 (s, 3H), 2.80 (s, 3H), 1.64-1.57 (m, 1H), 1.19-1.11 (m, 2H), 0.91-0.84 (m, 2H). ¹⁹F NMR (376 MHz, Chloroform-d) δ −63.93.

### 1.4 2-(4-cyclopropyl-6-methoxy-pyrimidin-5-yl)-4-methyl-7-[[4-[1-methyl-4-(trifluoromethyl)imidazol-2-yl]phenyl]methyl]-5H-pyrrolo[3,2-d]pyrimidine

To a solution of [2-(4-cyclopropyl-6-methoxy-pyrimidin-5-yl)-4-methyl-5H-pyrrolo[3,2-d]pyrimidin-7-yl]-[4-[1-methyl-4-(trifluoromethyl)imidazol-2-yl]phenyl]methanol (30 mg, 56 μmol) and triethylsilane (437 mg, 3.76 mmol, 0.6 mL) in chloroform (0.6 mL) under nitrogen atmosphere was added trifluoroacetic acid (444 mg, 3.89 mmol, 0.3 mL) at 0° C. The reaction mixture was stirred at 25° C. for 3 hours and then concentrated under reduced pressure. The residue was purified by Prep-TLC, eluted with 10% methanol in dichloromethane to give a crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 25 mL/min; Gradient: 5% B to 5% B in 2 min, 5% B to 40% B in 20 min; 40% B to 40% B in 2 min, 40% B to 95% B in 10 min. Detector: UV 254 & 210 nm; RT: 23 min. The collected fractions were combined, concentrated and then lyophilized overnight to give 2-(4-cyclopropyl-6-methoxy-pyrimidin-5-yl)-4-methyl-7-[[4-[1-methyl-4-(trifluoromethyl)imidazol-2-yl]phenyl]methyl]-5H-pyrrolo[3,2-d]pyrimidine (8.4 mg, 16.17 μmol, 29% yield) as an off-white solid. MS: m/z=520.30 [M+H]⁺. ¹H NMR (400 MHz, Chloroform-d) δ 9.68 (s, 1H), 8.64 (s, 1H), 7.53 (d, J=8.4 Hz, 2H), 7.41 (d, J=8.0 Hz, 2H), 7.36 (s, 1H), 7.13 (s, 1H), 4.26 (s, 2H), 3.91 (s, 3H), 3.78 (s, 3H), 2.74 (s, 3H), 1.70-1.64 (m, 1H), 1.27-1.19 (m, 2H), 0.90-0.79 (m, 2H). F NMR (376 MHz, Chloroform-d) δ −62.48.

### Example 67, 5-(2-cyclopropyl-4-methoxy-3-pyridyl-3-[[4-[1-methyl-4-(trifluoromethyl)imidazol-2-yl]phenyl]methyl]-1H-pyrazolo[4,3-d]pyrimidine Compound 116

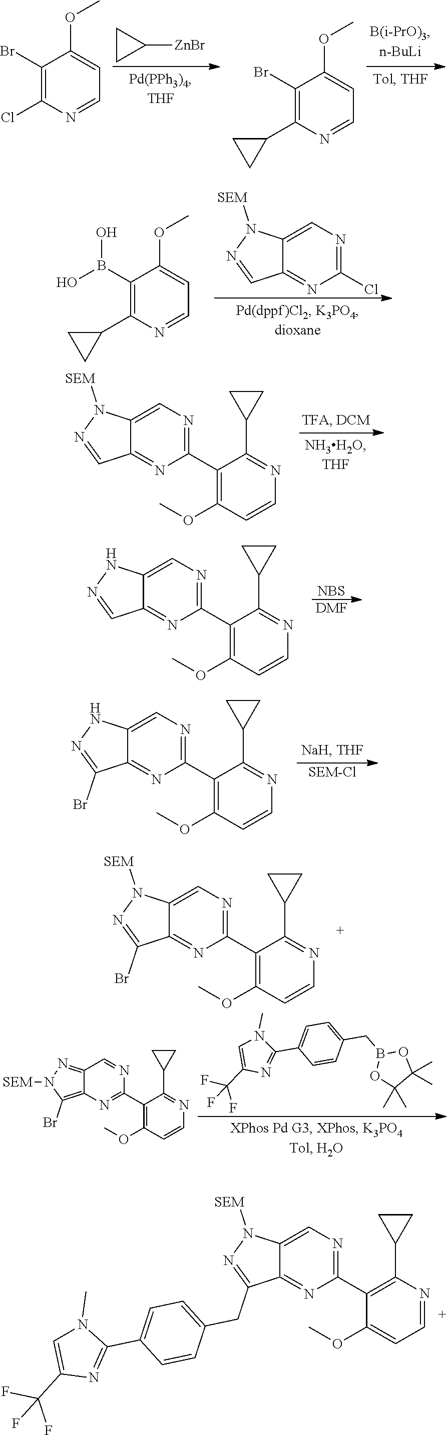

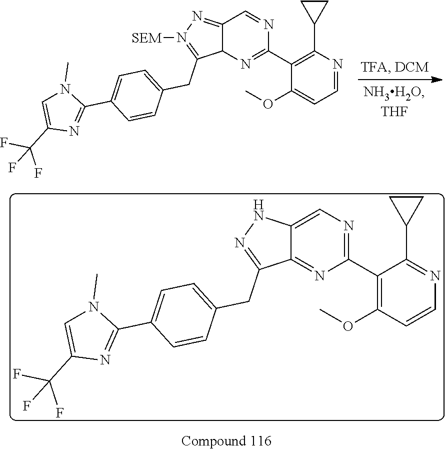

### 1.1 3-bromno-2-cyclopropyl-4-methoxy-pyridine

To a solution of 3-bromo-2-chloro-4-methoxy-pyridine (10 g, 44.95 mmol) in tetrahydrofuran (50 mL) was added cyclopropylzinc bromide (98.9 mL, 0.5 M in THE 49.45 mmol) and tetrakis(triphenylphosphine)palladium (1.56 g, 1.35 mmol) at 15 TC. The above solution was stirred at 65° C. for 16 hours. The resulted solution was detected by LCMS, could find 30% desired product overlap with by-product. The resulted solution was quenched by saturated ammonium chloride aqueous solution (400 mL), extracted by ethyl acetate (300 mL×3). The combined organic layers were dried over anhydrous sodium sulfate. After filtration, the filtrate was concentrated under reduced pressure. The residue was purified by silica gel column chromatography, eluted with 10%-25% ethyl acetate in petroleum ether to afford 3-bromo-2-cyclopropyl-4-methoxy-pyridine (1.6 g, 7.01 mmol, 15% yield) as a white solid. MS: m/z=228.00, 230.00 [M+H]⁺. ¹H NMR (400 MHz, Chloroform-d) δ 8.25 (d, J=5.6 Hz, 11H), 6.62 (d. J=5.6 Hz, 1H), 3.96 (s, 3H), 2.64-2.57 (m, 1H), 1.13-1.10 (m, 2H), 1.09-0.98 (m, 2H).

### 1.2 (2-cyclopropyl-4-methoxy-3-pyridyl)boronic Acid

To a solution of 3-bromo-2-cyclopropyl-4-methoxy-pyridine (800 mg, 3.51 mmol) in toluene (8 mL) and tetrahydrofuran (2 mL) was added triisopropyl borate (858 mg, 4.56 mmol) at 15° C. under nitrogen atmosphere, then was cooled down to −70° C. The resulting solution was added n-butyllithium (2.24 mL, 2.5 M in Hexane, 5.61 mmol) dropwise for 1.5 hours with stirring at −70° C. under nitrogen atmosphere. The resulting solution was stirred at −70° C. for 20 minutes under nitrogen atmosphere. The resulted solution was quenched by 1 M hydrochloric acid aqueous solution (3.16 mL), naturally warmed up to 15° C. The mixture solution was detected by LCMS, could find 27% desired product. The resulted solution was concentrated under reduced pressure directly. The residue was purified by RP-Flash chromatography with the following conditions: Column: C18 spherical, 20-30 um, 100A, 120 g; Mobile Phase A: 0.25 mM aq. hydrochloric acid. Mobile Phase B: acetonitrile; Flow rate: 100 mL/min; Gradient: 0% B to 0% B in 15 min, 0% B to 10% B in 20 min, 10% B to 25% B in 5 min, 25% B to 95% B in 5 min; Detector: UV 254 & 220 nm: RT: 16 min to afford (2-cyclopropyl-4-methoxy-3-pyridyl)boronic acid (510 mg, crude) as a white solid. MS: m/z=194.05 [M+H]⁺.

### 1.3 2-[[5-(2-cyclopropyl-4-methoxy-3-pyridyl)pyrazolo[4,3-d]pyrimidin-1-yl]methoxy]ethyl-trimethyl-silane

To a solution of (2-cyclopropyl-4-methoxy-3-pyridyl)boronic acid (510 mg, 2.64 mmol) in 1,4-dioxane (20 mL) were added 2-[(5-chloropyrazolo[4,3-d]pyrimidin-1-yl)methoxy]ethyl-trimethyl-silane (753 mg, 2.64 mmol), [1,1′-Bis(diphenylphosphino)ferrocene]dichloropalladium(II) (216 mg, 264 μmol) and potassium phosphate tribasic (1.68 g, 7.93 mmol) at 15° C. under nitrogen atmosphere. The above solution was stirred at 100° C. for 16 hours under nitrogen atmosphere. The resulted solution was detected by LCMS, could find 38% desired product. The resulted solution was filtrated. The filtrate was concentrated under reduced pressure. The residue was purified by silica gel column chromatography, eluted with 25%-70% ethyl acetate in petroleum ether to give 2-[[5-(2-cyclopropyl-4-methoxy-3-pyridyl)pyrazolo[4,3-d]pyrimidin-1-yl]methoxy]ethyl-trimethyl-silane (285 mg, 716.90 μmol, 27% yield) as a brown oil. MS: m/z=398.10 [M+H]⁺. ¹H NMR (400 MHz, Chloroform-d) δ 9.43 (s, 1H), 8.48 (d, J=6.0 Hz, 1H), 8.35 (s, 1H), 6.80 (s, 1H), 5.89 (s, 2H), 3.81 (s, 3H), 3.71-3.62 (m, 2H), 1.32-1.26 (m, 1H), 1.24-1.17 (m, 2H), 1.02-0.82 (m, 4H), −0.01 (s, 9H).

### 1.4 5-(2-cyclopropyl-4-methoxy-3-pyridyl)-1H-pyrazolo[4,3-d]pyrimidine

To a solution of 2,2,2-trifluoroacetic acid (3 mL) in dichloromethane (3 mL) was added 2-[[5-(2-cyclopropyl-4-methoxy-3-pyridyl)pyrazolo[4,3-d]pyrimidin-1-yl]methoxy]ethyl-trimethyl-silane (285 mg, 716.9 μmol) at 15° C. The above solution was stirred at 15° C. for 2 hours. The resulted solution was concentrated under reduced pressure. The residue was added tetrahydrofuran (3 mL) and ammonia solution (28% in H₂O, 3 mL). The above solution was stirred at 25° C. for 10 min. The resulted solution was detected by LCMS, major desired product. The resulted solution was concentrated under reduced pressure. The residue was purified by silica gel column chromatography, eluted with 0%-15% methanol in dichloromethane to give 5-(2-cyclopropyl-4-methoxy-3-pyridyl)-1H-pyrazolo[4,3-d]pyrimidine (180 mg, 673.44 μmol, 93% yield) as a yellow solid. MS: m/z=268.10 [M+H]⁺. ¹H NMR (400 MHz, Chloroform-d) δ 11.01 (br., 1H), 9.39 (s, 1H), 8.51 (d, J=6.0 Hz, 1H), 8.45 (s, 1H), 6.83 (d, J=6.0 Hz, 1H), 3.83 (s, 3H), 1.29-1.25 (m, 3H), 0.88-0.85 (m, 2H).

### 1.5 3-bromo-5-(2-cyclopropyl-4-methoxy-3-pyridyl)-1H-pyrazolo[4,3-d]pyrimidine

To a solution of 5-(2-cyclopropyl-4-methoxy-3-pyridyl)-1H-pyrazolo[4,3d]pyrimidine (180 mg, 673 μmol) in N,N-dimethyl formamide (4 mL) was added N-Bromosuccinimide (120 mg, 673 μmol) at 15° C., then was stirred at 15° C. for 6 hours. The resulted solution was detected by LCMS, major desired product. The resulted solution was diluted by ethyl acetate (100 mL). The organic layer was washed with saturated sodium chloride aqueous solution (30 mL×3). The combined organic layers were dried over anhydrous sodium sulfate. After filtration, the filtrate was concentrated under reduced pressure. The residue was purified by silica gel column chromatography, eluted with 0%-15% methanol in dichloromethane to afford 3-bromo-5-(2-cyclopropyl-4-methoxy-3-pyridyl)-1H-pyrazolo[4,3-d]pyrimidine (169 mg, 488.18 μmol, 72% yield) as a yellow solid. MS: m/z=346.00, 348.00 [M+H]⁺. ¹H NMR (400 MHz, DMSO-d₆) δ 14.37 (br., 1H), 9.49 (s, 1H), 8.40 (d, J=6.0 Hz, 1H), 6.98 (d, J=6.0 Hz, 1H), 3.72 (s, 3H), 1.38-1.34 (m, 1H), 0.96-0.93 (m, 2H), 0.72-0.68 (m, 2H).

### 1.6 2-[[3-bromo-5-(2-cyclopropyl-4-methoxy-3-pyridyl)pyrazolo[4,3-d]pyrimidin-1-yl]methoxy]ethyl-trimethyl-silane & 2-[[3-bromo-5-(2-cyclopropyl-4-methoxy-3-pyridyl)pyrazolo[4,3-d]pyrimidin-2-yl]methoxy]ethyl-trimethyl-silane

To a solution of 3-bromo-5-(2-cyclopropyl-4-methoxy-3-pyridyl)-1H-pyrazolo[4,3-d]pyrimidine (169 mg, 488.18 μmol) in tetrahydrofuran (4 mL) was added sodium hydride (23 mg, 586 μmol, 60% dispersion in mineral oil) at 0° C., then was stirred at 15° C. for 1 hours. The resulting solution was added 2-(chloromethoxy)ethyl-trimethyl-silane (81 mg, 488 μmol) at 15° C. The above solution was stirred at 15° C. for 3 hours. The resulted solution was detected by LCMS, major desired product. The resulted solution was quenched by saturated ammonium chloride aqueous solution (50 mL), extracted by ethyl acetate (30 mL×3). The combined organic layers were dried over anhydrous sodium sulfate. After filtration, the filtrate was concentrated under reduced pressure. The residue was purified by silica gel column chromatography, eluted with 0-15% methanol in dichloromethane to give a mixture of 2-[[3-bromo-5-(2-cyclopropyl-4-methoxy-3-pyridyl)pyrazolo[4,3-d]pyrimidin-1-yl]methoxy]ethyl-trimethyl-silane & 2-[[3-bromo-5-(2-cyclopropyl-4-methoxy-3-pyridyl)pyrazolo[4,3-d]pyrimidin-2-yl]methoxy]ethyl-trimethyl-silane (163 mg, 342.12 μmol, 70% yield) as a yellow solid. MS: m/z=476.00, 478.00 [M+H]⁺.

### 1.72-[[5-(2-cyclopropyl-4-methoxy-3-pyridyl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane & 2-[[5-(2-cyclopropyl-4-methoxy-3-pyridyl)-3-[[4-[1-methyl-4-(trifluoromethyl)imidazol-2-yl]phenyl]methyl]pyrazolo[4,3-d]pyrimidin-2-yl]methoxy]ethyl-trimethyl-silane

To a solution of 2-[[3-bromo-5-(2-cyclopropyl-4-methoxy-3-pyridyl)pyrazolo[4,3-d]pyrimidin-1-yl]methoxy]ethyl-trimethyl-silane & 2-[[3-bromo-5-(2-cyclopropyl-4-methoxy-3-pyridyl)pyrazolo[4,3-d]pyrimidin-2-yl]methoxy]ethyl-trimethyl-silane (130 mg, 272.86 μmol) in toluene (6.5 mL) and water (1.3 mL) were added 1-methyl-2-[4-[(4,4,5,5-tetramethyl-1,3,2-dioxaborolan-2-yl)methyl]phenyl]-4-(trifluoromethyl)imidazole (200 mg, 546 μmol), potassium phosphate (116 mg, 546 μmol), 2-Dicyclohexylphosphino-2′,4′,6′-triisopropylbiphenyl (13 mg, 27 μmol) and methanesulfonato(2-dicyclohexylphosphino-2′,4′,6′-tri-i-propyl-1,1′-biphenyl)(2′-amino-1,1′-biphenyl-2-yl)palladium(II) (23 mg, 27 μmol) at 15° C. The above solution was stirred at 90° C. for 16 hours. The resulted solution was detected by LCMS, could find 52% desired product. The resulted solution was concentrated under reduced pressure. The residue was quenched by water (100 mL), extracted by ethyl acetate (150 mL×3). The organic layer was dried over anhydrous sodium sulfate. After filtration, the filtrate was concentrated under reduced pressure. The residue was purified by Prep-TLC (dichloromethane:methanol=12:1) to afford 2-[[5-(2-cyclopropyl-4-methoxy-3-pyridyl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane & 2-[[5-(2-cyclopropyl-4-methoxy-3-pyridyl)-3-[[4-[1-methyl-4-(trifluoromethyl)imidazol-2-yl]phenyl]methyl]pyrazolo[4,3-d]pyrimidin-2-yl]methoxy]ethyl-trimethyl-silane (110 mg, crude) as a yellow oil, which was used in the next step without purification. MS: m/z=636.25 [M+H]⁺.

### 1.8 5-(2-cyclopropyl-4-methoxy-3-pyridyl)-3-[[4-[1-methyl-4-(trifluoromethyl)imidazol-2-yl]phenyl]methyl]-1H-pyrazolo[4,3-d]pyrimidine

To a solution of 2,2,2-trifluoroacetic acid (2 mL) in dichloromethane (2 mL) was added 2-[[5-(2-cyclopropyl-4-methoxy-3-pyridyl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane & 2-[[5-(2-cyclopropyl-4-methoxy-3-pyridyl)-3-[[4-[1-methyl-4-(trifluoromethyl)imidazol-2-yl]phenyl]methyl]pyrazolo[4,3-d]pyrimidin-2-yl]methoxy]ethyl-trimethyl-silane (110 mg, 103 μmol, 52% purity) at 15° C. The above solution was stirred at 15° C. for 2 hours. The resulted solution was concentrated under reduced pressure. The residue was added tetrahydrofuran (2 mL) and ammonia solution (28% in H₂O, 2 mL), then was stirred at 15° C. for 10 min. The resulted solution was detected by LCMS, major was desired product. The resulted solution was concentrated under reduced pressure. The residue was purified by RP-Flash chromatography with the following conditions: Column: C18 spherical, 20-35 um, 100A, 20 g; Mobile Phase A: 10 mM aq. ammonium bicarbonate, Mobile Phase B: acetonitrile; Flow rate: 20 mL/min; Gradient: 5% B to 5% B in 5 min, 5% B to 25% B in 5 min, 25% B to 40% B in 15 min, 40% B to 95% B in 5 min; Detector: UV 254 & 220 nm: RT: 17 min to give 5-(2-cyclopropyl-4-methoxy-3-pyridyl)-3-[[4-[1-methyl-4-(trifluoromethyl)imidazol-2-yl]phenyl]methyl]-1H-pyrazolo[4,3-d]pyrimidine (10.3 mg, 20.38 μmol, 19% yield) as a white solid. MS: m/z=506.30 [M+H]⁺. ¹H NMR (400 MHz, Chloroform-d) δ 13.13 (br., 1H), 9.18 (s, 1H), 8.45 (d, J=6.0 Hz, 1H), 7.66 (d, J=8.0 Hz, 2H), 7.60 (d, J=8.0 Hz, 2H), 7.39 (s, 1H), 6.78 (d, J=6.0 Hz, 1H), 4.56 (s, 2H), 3.81 (s, 3H), 3.79 (s, 3H), 1.58-1.45 (m, 1H), 1.19-1.13 (m, 2H), 0.75-0.68 (m, 2H). ¹⁹F NMR (376 MHz, Chloroform-d) δ −62.25

### Example 68. 5-(4-cyclopropyl-6-methoxy-pyrimidin-5-yl)-3-[[4-[5-ethoxy-3-(trifluoromethyl)pyrazol-1-yl]phenyl]methyl]-1H-pyrazolo[4,3-d]pyrimidine Compound 123

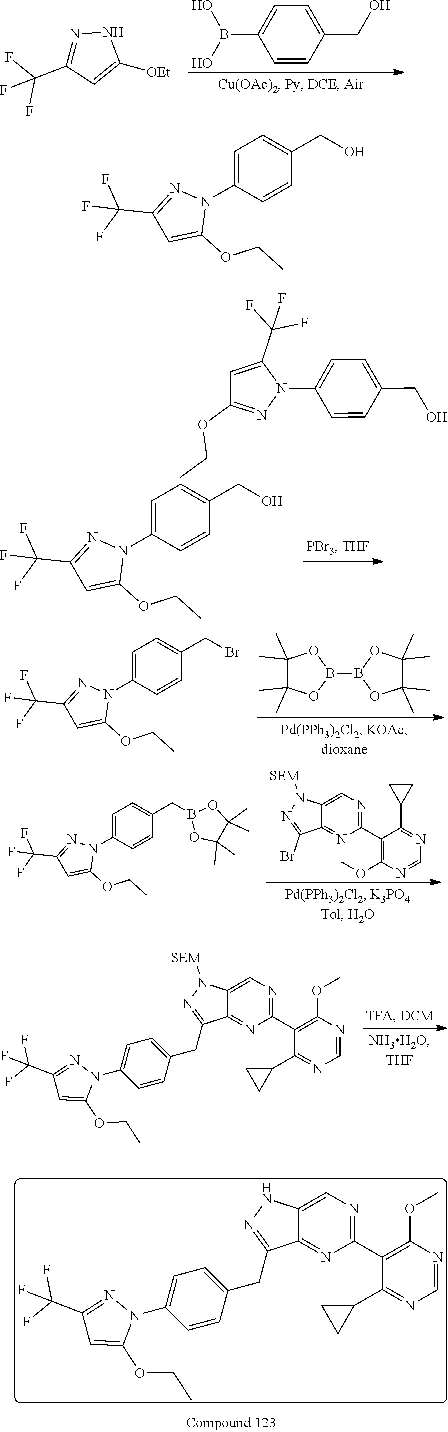

### 1.1 [4-[3-ethoxy-5-(trifluoromethyl)pyrazol-1-yl]phenyl]methanol & [4-[5-ethoxy-3-(trifluoromethyl)pyrazol-1-yl]phenyl]methanol

To a stirring solution of 5-ethoxy-3-(trifluoromethyl)-1H-pyrazole (1 g, 5.55 mmol) and [4-(hydroxymethyl)phenyl]boronic acid (1.69 g, 11.10 mmol) in 1,2-dichloroethane (10 mL) were added cupric acetate (2.01 g, 11.10 mmol) and pyridine (2.20 g, 27.76 mmol, 2.25 mL) at 25° C. under air atmosphere. The resulting mixture was stirred at 60° C. for 16 hours under air atmosphere. The reaction was quenched by the addition of ammonium hydroxide (1 mL, 28% aqueous solution) at 0° C., and then stirred at 25° C. for 0.5 hour. The resulted mixture was diluted with 20 mL water, extracted with ethyl acetate (3×20 mL). The combined organic layers were washed with brine (10 mL), dried over anhydrous sodium sulfate. After filtration, the filtrate was concentrated under reduced pressure. The residue was purified by silica gel column chromatography, eluted with 1%-50% ethyl acetate in petroleum ether to give two isomers. The small polar isomer collected fractions were concentrated under reduced pressure to give [4-[5-ethoxy-3-(trifluoromethyl)pyrazol-1-yl]phenyl]methanol (60 mg, 209.61 μmol, 4% yield,) as a light-yellow oil. MS: m/z=287.15 [M+H]⁺. ¹H NMR (400 MHz, Chloroform-d) δ 7.47 (s, 4H), 6.24 (s, 1H), 4.77 (s, 2H), 4.32-4.27 (m, 2H), 1.43 (t, J=7.2 Hz, 3H).

The big polar isomer collected fractions were concentrated under reduced pressure to give [4-[3-ethoxy-5-(trifluoromethyl)pyrazol-1-yl]phenyl]methanol (850 mg, 2.97 mmol, 53% yield,) as a light-yellow oil. MS: m/z=287.05 [M+H]⁺. ¹H NMR (400 MHz, Chloroform-d) δ 7.73-7.70 (m, 2H), 7.47-7.44 (m, 2H), 5.94 (s, 1H), 4.75 (s, 2H), 4.26-4.21 (m, 2H), 1.43 (t, J=7.2 Hz, 3H).

### 1.2 1-[4-(bromomethyl)phenyl]-5-ethoxy-3-(trifluoromethyl)pyrazole

To a solution of [4-[5-ethoxy-3-(trifluoromethyl)pyrazol-1-yl]phenyl]methanol (600 mg, 2.10 mmol) in tetrahydrofuran (6 mL) was added tribromophosphane (2.84 g, 10.48 mmol) dropwise with stirring at 0° C., and then stirred at 25° C. for 2 hours under nitrogen atmosphere. The reaction was quenched by the addition of saturated sodium bicarbonate aqueous solution (20 mL) at 0° C. The resulting mixture was extracted with ethyl acetate (3×30 mL). The combined organic layers were washed with brine (10 mL), dried over anhydrous sodium sulfate. After filtration, the filtrate was concentrated under reduced pressure. The residue was purified by silica gel column chromatography, eluted with 1%-50% ethyl acetate in petroleum ether to give 1-[4-(bromomethyl)phenyl]-5-ethoxy-3-(trifluoromethyl)pyrazole (400 mg, 1.15 mmol, 55% yield) as a white solid. MS: m/z=348.85, 350.85 [M+H]⁺. ¹H NMR (400 MHz, Chloroform-d) δ 7.76-7.72 (m, 2H), 7.51-7.48 (m, 2H), 5.94 (s, 1H), 4.54 (s, 2H), 4.27-4.22 (m, 2H), 1.50 (t, J=7.2 Hz, 3H).

### 1.3 5-ethoxy-1-[4-[(4,4,5,5-tetramethyl-1,3,2-dioxaborolan-2-yl)methyl]phenyl]-3-(trifluoromethyl)pyrazole

To a stirring solution of 1-[4-(bromomethyl)phenyl]-5-ethoxy-3-(trifluoromethyl)pyrazole (540 mg, 1.55 mmol) and 4,4,5,5-tetramethyl-2-(4,4,5,5-tetramethyl-1,3,2-dioxaborolan-2-yl)-1,3,2-dioxaborolane (589 mg, 2.32 mmol) in 1,4-dioxane (5 mL) were added bis(triphenylphosphine)palladium(II) chloride (109 mg, 155 μmol) and potassium acetate (455 mg, 4.64 mmol) at 25° C. under nitrogen atmosphere and then stirred at 80° C. for 2 hours. The resulting mixture was filtered. The filter cake was washed with ethyl acetate (3×5 mL). The combined organic layers were concentrated under reduced pressure. The residue was purified by silica gel column chromatography, eluted with 1%-30% ethyl acetate in petroleum ether to give 5-ethoxy-1-[4-[(4,4,5,5-tetramethyl-1,3,2-dioxaborolan-2-yl)methyl]phenyl]-3-(trifluoromethyl)pyrazole (141 mg, 355.87 μmol, 23% yield) as a light yellow solid. MS: m/z=397.30 [M+H]⁺. ¹H NMR (400 MHz, Chloroform-d) δ 7.58-7.55 (m, 2H), 7.28-7.26 (m, 2H), 5.92 (s, 1H), 4.24-4.19 (m, 2H), 2.34 (s, 2H), 1.47 (t, J=7.2 Hz, 3H), 1.25 (s, 12H).

### 1.4 5-(4-cyclopropyl-6-methoxy-pyrimidin-5-yl)-3-[[4-[5-ethoxy-3-(trifluoromethyl)pyrazol-1-yl]phenyl]methyl]-1H-pyrazolo[4,3-d]pyrimidine

To a stirred solution of 5-ethoxy-1-[4-[(4,4,5,5-tetramethyl-1,3,2-dioxaborolan-2-yl)methyl]phenyl]-3-(trifluoromethyl)pyrazole (94 mg, 237.25 μmol) and 2-[[3-bromo-5-(4-cyclopropyl-6-methoxy-pyrimidin-5-yl)pyrazolo[4,3-d]pyrimidin-1-yl]methoxy]ethyl-trimethyl-silane (125 mg, 261 μmol) in water (0.4 mL) and toluene (1.6 mL) were added potassium phosphate (101 mg, 474.5 μmol) and bis(triphenylphosphine)palladium(IT) chloride (67 mg, 94.9 μmol) at 25° C. under nitrogen atmosphere. The resulting mixture was stirred at 90° C. for 16 hours under nitrogen atmosphere. The resulted mixture was quenched with water (25 mL). The mixture was extracted with ethyl acetate (3×25 mL). The combined organic layers were washed with brine (10 mL), dried over anhydrous sodium sulfate. After filtration, the filtrate was concentrated under reduced pressure. The residue was purified by silica gel column chromatography, eluted with 1%-50% ethyl acetate in petroleum ether to give 5-(4-cyclopropyl-6-methoxy-pyrimidin-5-yl)-3-[[4-[5-ethoxy-3-(trifluoromethyl)pyrazol-1-yl]phenyl]methyl]-1H-pyrazolo[4,3-d]pyrimidine (50 mg, 93.20 μmol, 39% yield) as a white solid. MS: m/z=667.50 [M+H]+.

### 1.5 5-(4-cyclopropyl-6-methoxy-pyrimidin-5-yl)-3-[[4-[5-ethoxy-3-(trifluoromethyl)pyrazol-1-yl]phenyl]methyl]-1H-pyrazolo[4,3-d]pyrimidine

To a stirred solution of 2-[[5-(4-cyclopropyl-6-methoxy-pyrimidin-5-yl)-3-[[4-[5-ethoxy-3-(trifluoromethyl)pyrazol-1-yl]phenyl]methyl]pyrazolo[4,3-d]pyrimidin-1-yl]methoxy]ethyl-trimethyl-silane (50 mg, 74.99 μmol) in dichloromethane (1.50 mL) was added 2,2,2-trifluoroacetic acid (740 mg, 6.49 mmol, 500 μL) at 25° C. under nitrogen atmosphere and then stirred at this temperature 1 hour. The resulting mixture was concentrated under reduced pressure. To a stirred solution of the residue in tetrahydrofuran (1.50 mL) was added ammonium hydroxide (28% aqueous, 1.5 mL) at 25° C. under nitrogen atmosphere and then stirred at this temperature for 1 hour. The resulted mixture was concentrated under reduced pressure. The obtained product was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 45% B in 10 min; 45% B to 45% B in 5 min; 45% B to 95% B in 10 min. Detector: UV 254 & 210 nm; RT: 17 min.

The collected fractions were combined and lyophilized to give 5-(4-cyclopropyl-6-methoxy-pyrimidin-5-yl)-3-[[4-[5-ethoxy-3-(trifluoromethyl)pyrazol-1-yl]phenyl]methyl]-1H-pyrazolo[4,3-d]pyrimidine (17.6 mg, 32.80 μmol, 43% yield) as an off-white solid. MS: m/z=537.30 [M+H]⁺. ¹H NMR (400 MHz, Chloroform-d) δ 11.32 (br., 1H), 9.21 (s, 1H), 8.69 (s, 1H), 7.64 (d, J=8.8 Hz, 2H), 7.54 (d, J=8.4 Hz, 2H), 5.94 (s, 1H), 4.53 (s, 2H), 4.25-4.19 (m, 2H), 3.93 (s, 3H), 1.71-1.64 (m, 1H), 1.46 (t, J=6.8 Hz, 3H), 1.30-1.20 (m, 2H), 0.92-0.82 (m, 2H). ¹⁹F NMR (376 MHz, Chloroform-d) δ −63.12.

### Example 69. 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine Compound 94

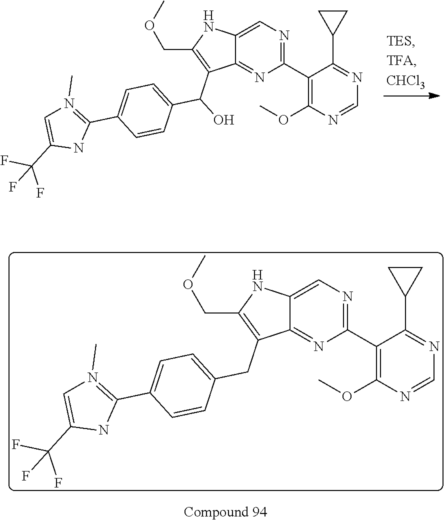

### 1.1 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine

A mixture of [2-(4-cyclopropyl-6-methoxy-pyrimidin-5-yl)-6-(methoxymethyl)-5H-pyrrolo[3,2-d]pyrimidin-7-yl]-[4-[1-methyl-4-(trifluoromethyl)imidazol-2-yl]phenyl]methanol (50 mg, 88.41 μmol) in chloroform (0.8 mL), trifluoroacetic acid (0.4 mL) and triethyl silane (0.8 mL) was stirred at room temperature for 36 hours. The reaction was concentrated directly under reduced pressure. The residue was purified by Prep-TLC (dichloromethane:methanol=20:1) to afford a crude product. The crude product was purified by reverse phase chromatography with the following conditions: Column: Xselect CSH OBD Column 30×150 mm 5 um; Mobile Phase A: ACN, Mobile Phase B: Water (5 mM aq. NH₄HCO₃); Flow rate: 60 mL/min; Gradient: 20% B to 35% B in 12 min, 35% B; Detector: UV 254 & 210 nm; RT: 11.20 min. The collected fractions were combined and concentrated under reduced pressure to afford 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine (25 mg, 45.49 μmol, 51% yield) as an off-white solid. MS: m/z=550.25 [M+H]⁺. ¹H NMR (400 MHz, Chloroform-d) δ 9.14 (s, 1H), 8.68 (s, 1H), 7.51 (d, J=8.0 Hz, 2H), 7.39 (d, J=8.0 Hz, 2H), 7.32 (s, 1H), 4.63 (s, 2H), 4.28 (s, 2H), 3.97 (s, 3H), 3.75 (s, 3H), 3.42 (s, 3H), 1.86-1.77 (m, 1H), 1.30-1.22 (m, 2H), 0.93-0.84 (m, 2H). ¹⁹F NMR (376 MHz, Chloroform-d) δ −62.71.

### Example 70. [2-(4-cyclopropyl-6-methoxy-pyrimidin-5-yl)-6-methyl-5H-pyrrolo[3,2-d]pyrimidin-7-yl]-[4-[1-methyl-4-(trifluoromethyl)imidazol-2-yl]phenyl]methanol Compound 207

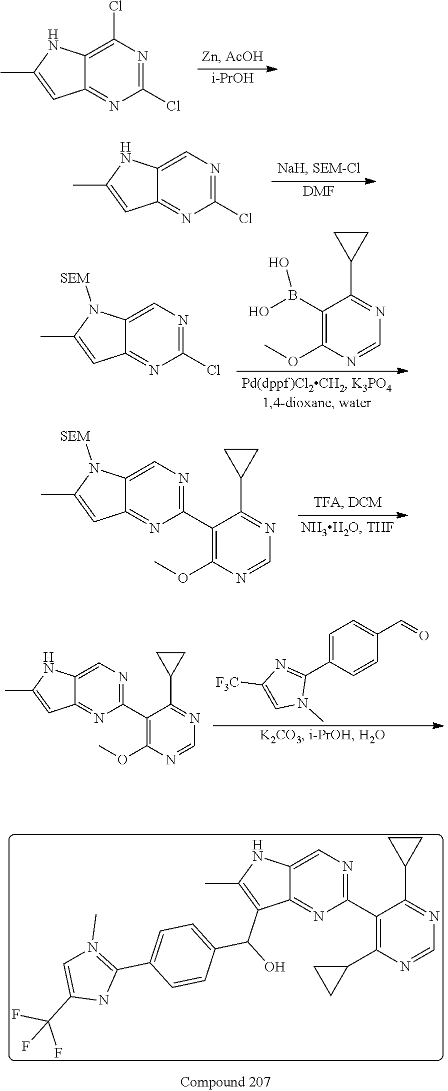

### 1.1 2-chloro-6-methyl-5H-pyrrolo[3,2-d]pyrimidine

To a stirred solution of 2,4-dichloro-6-methyl-5H-pyrrolo[3,2-d]pyrimidine (1.5 g, 7.42 mmol) in isopropyl alcohol (15 mL) were added acetic acid (2.67 g, 44.55 mmol, 2.55 mL) and zinc (1.94 g, 29.70 mmol) under nitrogen atmosphere at 25° C. The resulting mixture was stirred at 85° C. for 4 hours. The reaction mixture was cooled down to room temperature. The solid was filtered out and the filtrate was concentrated under reduced pressure. The residue was purified by silica gel column chromatography, eluted with 3%-4% methanol in dichloromethane to give 2-chloro-6-methyl-5H-pyrrolo[3,2-d]pyrimidine (800 mg, 4.77 mmol, 64% yield) as a yellow solid. MS: m/z=168.00, 170.00 [M+H]⁺. ¹H NMR (400 MHz, DMSO-d₆) 11.97 (s, 1H), 8.64 (s, 1H), 6.34 (s, 1H), 2.50 (s, 3H).

### 1.2 2-[(2-chloro-6-methyl-pyrrolo[3,2-d]pyrimidin-5-yl)methoxy]ethyl-trimethyl-silane

To a solution of 2-chloro-6-methyl-5H-pyrrolo[3,2-d]pyrimidine (800 mg, 4.77 mmol) in DMF (8 mL) under nitrogen atmosphere was added sodium hydride (179 mg, 2.57 mmol, 60% dispersion in oil) at 0° C. The resulting mixture was stirred at 0° C. for 1 hour. To the above mixture was added 2-(trimethylsilyl)ethoxymethyl chloride (796 mg, 4.8 mmol, 845 L) at 0° C., the resulting mixture was stirred at 25° C. for 2 hours. The resulted mixture was quenched by saturated ammonium chloride aqueous solution (30 mL), then extracted with ethyl acetate (2×30 mL). The combined organic layers were washed with brine (20 mL), dried over anhydrous sodium sulfate. After filtration, the filtrate was concentrated under reduced pressure. The residue was purified by silica gel column chromatography, eluted with 1%-2% methanol in dichloromethane to give 2-[(2-chloro-6-methyl-pyrrolo[3,2-d]pyrimidin-5-yl)methoxy]ethyl-trimethyl-silane (910 mg, 3.06 mmol, 64% yield) as a yellow solid. MS: m/z=298.05, 300.05 [M+H]⁺. ¹H NMR (400 MHz, Chloroform-d) δ 8.67 (s, 1H), 6.46 (s, 1H), 5.50 (s, 2H), 3.56-3.47 (m, 2H), 2.60 (s, 3H), 0.96-0.87 (m, 2H), −0.02 (s, 9H).

### 1.3 2-[[2-(4-cyclopropyl-6-methoxy-pyrimidin-5-yl)-6-methyl-pyrrolo[3,2-d]pyrimidin-5-yl]methoxy]ethyl-trimethyl-silane

To a solution of 2-[(2-chloro-6-methyl-pyrrolo[3,2-d]pyrimidin-5-yl)methoxy]ethyl-trimethyl-silane (910 mg, 3.06 mmol) and (4-cyclopropyl-6-methoxy-pyrimidin-5-yl)boronic acid (593 mg, 3.06 mmol) in 1,4-dioxane (8 mL) and water (2 mL) under nitrogen atmosphere were added potassium phosphate (1.30 g, 6.11 mmol) and [1,1′-Bis(diphenylphosphino)ferrocene]dichloropalladium(II) complex with dichloromethane (250 mg, 306 μmol) at 25° C. The resulting mixture was stirred at 100° C. for 16 hours. The reaction mixture was cooled down to room temperature. The solid was filtered out and the filtrate was concentrated under reduced pressure. The residue was purified by silica gel column chromatography, eluted with 40%-50% ethyl acetate in petroleum ether to give 2-[[2-(4-cyclopropyl-6-methoxy-pyrimidin-5-yl)-6-methyl-pyrrolo[3,2-d]pyrimidin-5-yl]methoxy]ethyl-trimethyl-silane (550 mg, 1.34 mmol, 43% yield) as a yellow solid. MS: m/z=412.20 [M+H]⁺. ¹H NMR (400 MHz, Chloroform-d) δ 8.98 (s, 1H), 8.67 (s, 1H), 6.58 (s, 1H), 5.57 (s, 2H), 3.93 (s, 3H), 3.65-3.57 (m, 2H), 2.64 (s, 3H), 1.73-1.62 (m, 1H), 1.30-1.20 (m, 2H), 1.00-0.93 (m, 2H), 0.93-0.84 (m, 2H), 0.00 (s, 9H).

### 1.4 2-(4-cyclopropyl-6-methoxy-pyrimidin-5-yl)-6-methyl-5H-pyrrolo[3,2-d]pyrimidine

To a solution of 2-[[2-(4-cyclopropyl-6-methoxy-pyrimidin-5-yl)-6-methyl-pyrrolo[3,2-d]pyrimidin-5-yl]methoxy]ethyl-trimethyl-silane (550 mg, 1.34 mmol) in DCM (5 mL) was added trifluoroacetic acid (5 mL) at 25° C., then stirred at 25° C. for 2 hours. The reaction mixture was concentrated under reduced pressure. The residue was dissolved in tetrahydrofuran (5 mL) and ammonium hydroxide (5 mL) at 0° C., and then stirred at 25° C. for 10 min. The reaction mixture was concentrated under reduced pressure. The residue was purified by silica gel column chromatography, eluted with 2%-3% methanol in dichloromethane to give 400 mg crude. The crude product was purified by RP-Flash with the following conditions: C18 spherical Column, 20-35 um, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 50% B in 10 min, 50% B to 50% B in 3 min, 30% B to 95% B in 10 min; Detector: UV 254 & 210 nm; RT: 17 min. The fractions were combined and concentrated under reduced pressure to give 2-(4-cyclopropyl-6-methoxy-pyrimidin-5-yl)-6-methyl-5H-pyrrolo[3,2-d]pyrimidine (305 mg, 1.08 mmol, 81% yield) as an off-white solid. MS: m/z=282.10 [M+H]⁺. ¹H NMR (400 MHz, Chloroform-d) δ 11.82 (s, 1H), 8.83 (s, 1H), 8.64 (s, 1H), 6.38 (s, 1H), 3.81 (s, 3H), 2.52 (s, 3H), 1.58-1.52 (m, 1H), 1.03-(m, 2H), 0.86-0.81 (m, 2H).

### 1.5 [2-(4-cyclopropyl-6-methoxy-pyrimidin-5-yl)-6-methyl-5H-pyrrolo[3,2-d]pyrimidin-7-yl]-[4-[1-methyl-4-(trifluoromethyl)imidazol-2-yl]phenyl]methanol

To a solution of 2-(4-cyclopropyl-6-methoxy-pyrimidin-5-yl)-6-methyl-5H-pyrrolo[3,2-d]pyrimidine (189 mg, 671.85 μmol) in isopropyl alcohol (2 mL) was added 4-[1-methyl-4-(trifluoromethyl)imidazol-2-yl]benzaldehyde (171 mg, 672 μmol) at 0° C. The resulting mixture was stirred at 0° C. for 10 min. To the above mixture were added potassium carbonate (111 mg, 806 μmol) and water (2 mL) at 0° C. The resulting mixture was stirred at 60° C. for 12 hours. The reaction mixture was diluted with water (20 mL), extracted with ethyl acetate (3×20 mL). The combined organic layers were washed with brine (20 mL), dried over anhydrous anhydrous sodium sulfate and filtered. The filtrate was concentrated under reduced pressure. The residue was purified by Prep-TLC (dichloromethane:methanol=10:1) to give 150 mg crude. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 35 mL/min; Gradient: 5% B to 5% B in 5 min, 5% B to 30% B in 5 min, 30% B to 30% B in 2 min, 30% B to 95% B in 10 min; Detector: UV 254 & 210 nm; RT: 11 min. The fractions were combined and concentrated under reduced pressure to give [2-(4-cyclopropyl-6-methoxy-pyrimidin-5-yl)-6-methyl-5H-pyrrolo[3,2-d]pyrimidin-7-yl]-[4-[1-methyl-4-(trifluoromethyl)imidazol-2-yl]phenyl]methanol (120 mg, 224.08 μmol, 33% yield) as an off-white solid. MS: m/z=536.20 [M+H]⁺. ¹H NMR (400 MHz, Chloroform-d) δ 10.51 (s, 1H), 8.93 (s, 1H), 8.67 (s, 1H), 7.59-7.54 (m, 4H), 7.37 (m, 1H), 6.26 (s, 1H), 4.78 (s, 1H), 3.95 (s, 3H), 3.78 (s, 3H), 2.25 (s, 3H), 1.83-1.78 (m, 1H), 1.23-1.18 (m, 2H), 0.92-0.84 (m, 2H); ¹⁹F NMR (376 MHz, Chloroform-d) −62.50.

### Example 71. 5-(4-cyclopropyl-6-methoxy-pyrimidin-5-yl)-3-[[4-[5-methoxy-3-(trifluoromethyl)pyrazol-1-yl]phenyl]methyl]-1H-pyrazolo[4,3-d]pyrimidine Compound 140

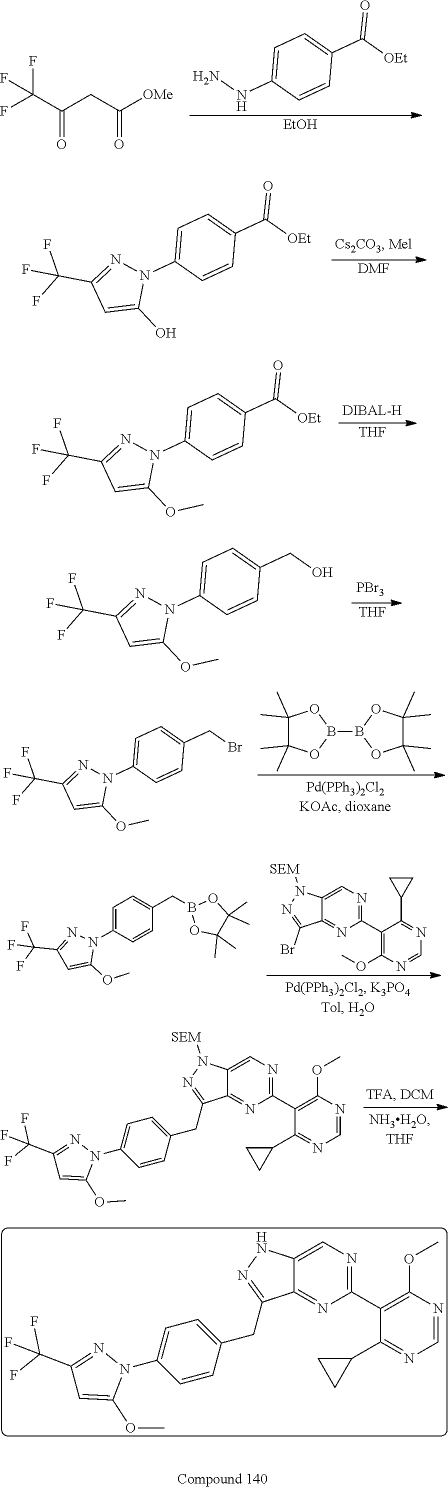

### 1.1 ethyl 4-[5-hydroxy-3-(trifluoromethyl)pyrazol-1-yl]benzoate

To a stirred solution of methyl 4,4,4-trifluoro-3-oxo-butanoate (2 g, 11.76 mmol, 1.49 mL) in ethanol (60 mL) was added ethyl 4-hydrazinobenzoate (2.12 g, 11.76 mmol) at 25° C. After the resulting mixture was stirred at 80° C. for 16 hours, it was cooled down to room temperature and concentrated under reduced pressure. The residue was purified by silica gel column chromatography, eluted with 1%-80% ethyl acetate in petroleum ether to give ethyl 4-[5-hydroxy-3-(trifluoromethyl)pyrazol-1-yl]benzoate (1.2 g, 4.00 mmol, 34% yield) as a little red solid. MS: m/z=300.85 [M+H]⁺.

### 1.2 ethyl 4-[5-methoxy-3-(trifluoromethyl)pyrazol-1-yl]benzoate

To a stirred solution of ethyl 4-[5-hydroxy-3-(trifluoromethyl)pyrazol-1-yl]benzoate (1.15 g, 3.83 μmol) and cesium carbonate (2.50 g, 7.66 mmol) in N,N-dimethylformamide (10 mL) was added iodomethane (652 mg, 4.6 mmol, 286 μL) at 0° C. under nitrogen atmosphere. After stirred at 25° C. for 16 hours, the reaction mixture was diluted with ethyl acetate (200 mL), washed with brine (3×50 mL). The organic layer was dried over anhydrous sodium sulfate. After filtration, the filtrate was concentrated under reduced pressure. The residue was purified by silica gel column chromatography, eluted with 1%-50% ethyl acetate in petroleum ether to give ethyl 4-[5-methoxy-3-(trifluoromethyl)pyrazol-1-yl]benzoate (500 mg, 1.59 mmol, 42% yield) as a red solid. MS: m/z=315.15 [M+H]⁺.

### 1.3 [4-[5-methoxy-3-(trifluoromethyl)pyrazol-1-yl]phenyl]methanol

To a stirred solution of ethyl 4-[5-methoxy-3-(trifluoromethyl)pyrazol-1-yl]benzoate (430 mg, 1.37 mmol) in THF (4 mL) was added diisobutylaluminum hydride (6.84 mL, 6.84 mmol, 1 N in THF) at 0° C. under nitrogen atmosphere. After stirred at 25° C. for 2 hours, the reaction mixture was diluted with ethyl acetate (50 mL) and the pH value of the solution was adjusted to 3˜4 with 1 N aq. hydrochloric acid. The resulted mixture was washed with brine (3×50 mL). The organic layer was dried over anhydrous sodium sulfate. After filtration, the filtrate was concentrated under reduced pressure. The residue was purified by silica gel column chromatography, eluted with 30%-70% ethyl acetate in petroleum ether to give [4-[5-methoxy-3-(trifluoromethyl)pyrazol-1-yl]phenyl]methanol (300 mg, 1.10 mmol, 81% yield) as a yellow solid. MS: m/z=273.05 [M+H]⁺. ¹H NMR (400 MHz, Chloroform-d) δ 7.70 (d, J=8.8 Hz, 2H), 7.48 (d, J=8.8 Hz, 2H), 5.98 (s, 1H), 4.77 (s, 2H), 4.01 (s, 3H). ¹⁹F NMR (376 MHz, Chloroform-d) δ −63.31.

### 1.4 1-[4-(bromomethyl)phenyl]-5-methoxy-3-(trifluoromethyl)pyrazole

To a stirred solution of [4-[5-methoxy-3-(trifluoromethyl)pyrazol-1-yl]phenyl]methanol (280 mg, 1.03 mmol) in THF (3 mL) under nitrogen atmosphere was added tribromophosphane (1392 mg, 5.14 mmol) at 0° C. After the resulting mixture was stirred at 25° C. for 16 hours, the reaction was quenched by the addition of saturated sodium bicarbonate aqueous solution (10 mL) at 0° C. The resulting mixture was diluted with ethyl acetate (50 mL), washed with brine (3×20 mL). The organic layer was dried over anhydrous sodium sulfate. After filtration, the filtrate was concentrated under reduced pressure. The residue was purified by silica gel column chromatography, eluted with 1%-30% ethyl acetate in petroleum ether to give 1-[4-(bromomethyl)phenyl]-5-methoxy-3-(trifluoromethyl)pyrazole (200 mg, 596.80 μmol, 58% yield) as a yellow solid. MS: m/z=334.95, 336.95 [M+H]⁺. ¹H NMR (400 MHz, Chloroform-d) δ 7.72 (d, J=8.8 Hz, 2H), 7.50 (d, J=8.4 Hz, 2H), 5.98 (s, 1H), 4.54 (s, 2H), 4.02 (s, 3H). ¹⁹F NMR (376 MHz, Chloroform-d) δ −63.34.

### 1.5 5-methoxy-1-[4-[(4,4,5,5-tetramethyl-1,3,2-dioxaborolan-2-yl)methyl]phenyl]-3-(trifluoromethyl)pyrazole

To a solution of 1-[4-(bromomethyl)phenyl]-5-methoxy-3-(trifluoromethyl)pyrazole (165 mg, 492.36 μmol) in 1,4-dioxane (1 mL) were added 4,4,5,5-tetramethyl-2-(4,4,5,5-tetramethyl-1,3,2-dioxaborolan-2-yl)-1,3,2-dioxaborolane (188 mg, 739 μmol), potassium acetate (145 mg, 1.48 mmol) and dichloropalladium triphenylphosphane (35 mg, 49 μmol) at 25° C. The reaction mixture was degassed with nitrogen for three times and then stirred at 80° C. for 16 hours. The resulted mixture was cooled down to room temperature and the solid was filtered out. The filtrate was concentrated under reduced pressure. The residue was purified by silica gel column chromatography, eluted with 1%-30% ethyl acetate in petroleum ether to give 5-methoxy-1-[4-[(4,4,5,5-tetramethyl-1,3,2-dioxaborolan-2-yl)methyl]phenyl]-3-(trifluoromethyl)pyrazole (100 mg, 261.65 μmol, 53% yield) as a yellow solid. MS: m/z=383.15 [M+H]⁺. ¹H NMR (400 MHz, Chloroform-d) δ 7.54 (d, J=8.4 Hz, 2H), 7.31-7.26 (m, 2H), 5.95 (s, 1H), 3.99 (s, 3H), 2.34 (s, 2H), 1.25 (s, 12H). ¹⁹F NMR (376 MHz, Chloroform-d) δ −63.19.

### 1.6 2-[[5-(4-cyclopropyl-6-methoxy-pyrimidin-5-yl)-3-[[4-[5-methoxy-3-(trifluoromethyl)pyrazol-1-yl]phenyl]methyl]pyrazolo[4,3-d]pyrimidin-1-yl]methoxy]ethyl-trimethyl-silane

To a solution of 5-methoxy-1-[4-[(4,4,5,5-tetramethyl-1,3,2-dioxaborolan-2-yl)methyl]phenyl]-3-(trifluoromethyl)pyrazole (80 mg, 209 μmol) in toluene (1 mL) and water (0.2 mL) were added 2-[[3-bromo-5-(4-cyclopropyl-6-methoxy-pyrimidin-5-yl)pyrazolo[4,3-d]pyrimidin-1-yl]methoxy]ethyl-trimethyl-silane (120 mg, 251 μmol), potassium phosphate (89 mg, 419 μmol) and dichloropalladium triphenylphosphane (15 mg, 21 μmol) at 25° C. The reaction mixture was degassed with nitrogen for 3 times and then stirred at 90° C. for 16 hours. The resulted mixture was cooled down to room temperature and the solid was filtered out. The filtrate was concentrated under reduced pressure. The residue was purified by silica gel column chromatography, eluted with 50%-100% ethyl acetate in petroleum ether to give 2-[[5-(4-cyclopropyl-6-methoxy-pyrimidin-5-yl)-3-[[4-[5-methoxy-3-(trifluoromethyl)pyrazol-1-yl]phenyl]methyl]pyrazolo[4,3-d]pyrimidin-1-yl]methoxy]ethyl-trimethyl-silane (40 mg, 61.3 μmol, 29% yield) as a little yellow solid. MS: m/z=653.25 [M+H]⁺. ¹H NMR (400 MHz, Chloroform-d) δ 9.60 (s, 1H), 8.72 (s, 1H), 7.61 (d, J=8.4 Hz, 2H), 7.40 (d, J=8.4 Hz, 2H), 5.95 (s, 1H), 5.75 (s, 2H), 4.69 (s, 2H), 3.99 (s, 3H), 3.98 (s, 3H), 3.65-3.55 (m, 2H), 1.86-1.55 (m, 1H), 1.03-0.79 (m, 6H), 0.02 (s, 9H). ¹⁹F NMR (376 MHz, Chloroform-d) − δ3.36, 1,75-(4-cyclopropyl-6-methoxy-pyrimidin-5-yl)-3-[[4-[5-methoxy-34trifluoromethyl)pyrazol-1-yl]phenyl]methyl]-1H-pyrazolo[4,3-d]pyrimidine

A solution of 2-[[5-(4-cyclopropyl-6-methoxy-pyrimidin-5-yl)-3-[[4-[5-methoxy-3-(trifluoromethyl)pyrazol-1-yl]phenyl]methyl]pyrazolo[4,3-d]pyrimidin-1-yl]methoxy]ethyl-trimethyl-silane (40 mg, 61.3 μmol) in DCM (1 mL) and trifluoroacetic acid (1 mL) was stirred at 25° C. for 1 hour and then concentrated under reduced pressure. The residue was dissolved in THF (1 mL) and ammonium hydroxide (1 mL) and then stirred at 25° C. for 30 min. The resulted mixture was concentrated under reduced pressure. The residue was purified by RP-Flash with the following conditions: Column: Spherical C18, 20-40 um, 40 g; Mobile Phase A: Water (5 mM aq. NH₄HCO₃), Mobile Phase B: MeCN; Flow rate: 40 mL/min; Gradient (B %): 2% B to 2% B in 5 min, 2% B to 62% B in 15 min, 62% B to 62% B in 3 min, 62% B to 95% B in 10 min, Detector: UV 254 & 210 nm; RT: 21 min. The collected fractions were combined and concentrated under reduced pressure to give 5-(4-cyclopropyl-6-methoxy-pyrimidin-5-yl)-3-[[4-[5-methoxy-3-(trifluoromethyl)pyrazol-1-yl]phenyl]methyl]-1H-pyrazolo[4,3-d]pyrimidine (9.1 mg, 17.42 μmol, 28% yield) as an off-white solid. MS: m/z=523.20 [M+H]⁺. ¹H NMR (400 MHz, Chloroform-d), β 9.31 (s, 1H), 8.73 (s, 1H), 7.58 (dd, J=20.0, 8.8 Hz, 4H), 5.95 (s, 1H), 4.54 (s, 2H), 3.98 (s, 3H), 3.96 (s, 3H), 1.75-1.66 (m, 1H), 1.32-1.26 (m, 2H), 0.92-0.87 (m, 2H). ¹⁹F NMR (376 MHz, Chloroform-d) δ −63.29.

### Example 72. 5-(4-cyclopropyl-6-methoxypyrimidin-5-yl)-3-(4-(1-methyl-4-(trifluoromethyl)-1H-imidazol-2-yl)benzyl)-1H-pyrazolo[3,4-c]pyridine Compound 177

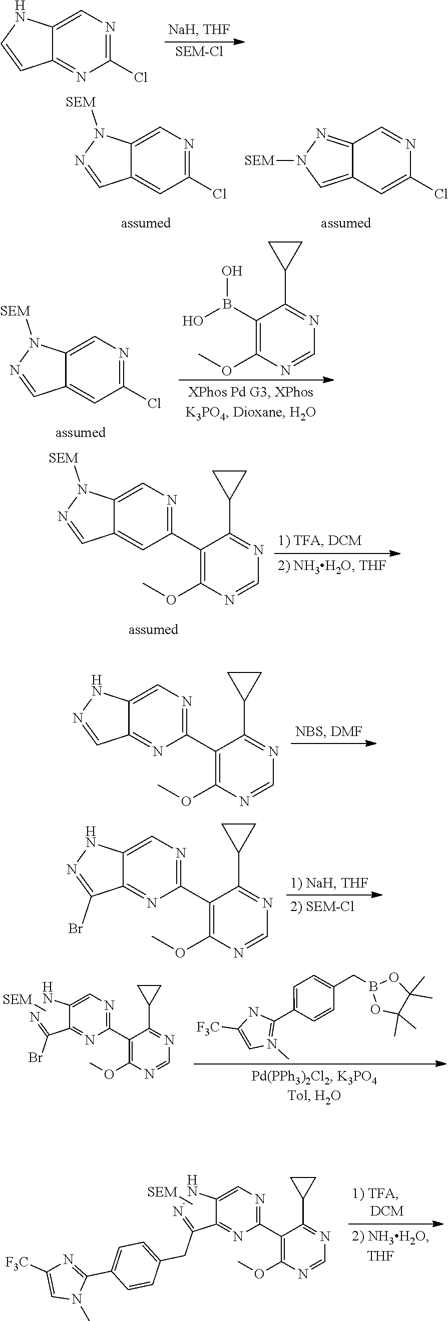

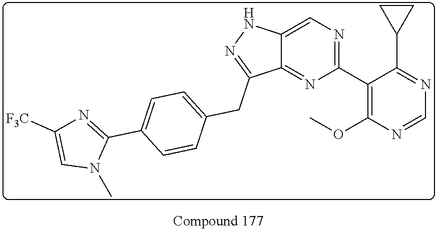 [SMILES: COc1ncnc(C2CC2)c1-c1ncc2[nH]nc(Cc3ccc(-c4nc(C(F)(F)F)cn4C)cc3)c2n1]

### 1.1 5-chloro-1-((2-(trimethylsilyl)ethoxy)methyl)-1H-pyrazolo[3,4-c]pyridine and 5-chloro-2-((2-(trimethylsilyl)ethoxy)methyl)-2H-pyrazolo[3,4-c]pyridine

To a solution of 5-chloro-1H-pyrazolo[3,4-c]pyridine (1.5 g, 9.77 mmol) in tetrahydrofuran (30 mL) was added sodium hydride (469 mg, 12 mmol, 60% dispersion in oil) at 0° C. under nitrogen atmosphere. The reaction mixture was stirred at 0° C. for 1 hour, then 2-(trimethylsilyl)ethoxymethyl chloride (1.95 g, 11.72 mmol) was added to the reaction mixture at 0′° C. The reaction was stirred at 25° C. for 16 hours. The reaction was quenched with saturated ammonium chloride aqueous solution (100 mL), and then extracted with ethyl acetate (3×100 mL). The combined organic extracts were washed with brine (100 mL), dried over anhydrous sodium sulfate. After filtration, the filtrate was concentrated under reduced pressure. The residue was purified by silica gel column chromatography, eluted with 1%-50% ethyl acetate in petroleum ether to give two isomers.

The small polar isomer fractions was concentrated under reduced pressure to give 5-chloro-2-((2-(trimethylsilyl)ethoxy)methyl)-2H-pyrazolo[3,4-c]pyridine (830 mg, 2.92 mmol, 30% yield, structure tentatively assigned) as yellow solid. MS: m/z=284.15, 286.15 [M+H]⁺. ¹H NMR (400 MHz, Chloroform-d) δ 8.91 (s, 1H), 8.04 (s, 1H), 7.69 (s, 1H), 5.82 (s, 2H), 3.60-3.51 (m, 2H), 0.95-0.85 (m, 2H), −0.05 (s, 8H).

The big polar isomer fractions was concentrated under reduced pressure to give 5-chloro-1-((2-(trimethylsilyl)ethoxy)methyl)-1H-pyrazolo[3,4-c]pyridine (880 mg, 3.10 mmol, 32% yield, structure tentatively assigned) as yellow solid. MS: m/z=284.20, 286.20 [M+H]⁺. ¹H NMR (400 MHz, Chloroform-d) δ 9.11 (s, 1H), 8.15 (s, 1H), 7.61 (s, 1H), 5.79 (s, 2H), 3.70-3.60 (m, 2H), 1.01-0.91 (m, 2H), 0.01 (s, 9H).

### 1.2 5-(4-cyclopropyl-6-methoxypyrimidin-5-yl)-1-((2-(trimethylsilylethoxy)methyl)-1H-pyrazolo[3,4-c]pyridine

To a mixture of 2-[(5-chloropyrazolo[3,4-c]pyridin-1-yl)methoxy]ethyl-trimethyl-silane (378 mg, 1.33 mmol) and (4-cyclopropyl-6-methoxy-pyrimidin-5-yl)boronic acid (388 mg, 2.0 mmol) in water (3 mL) and 1,4-dioxane (15 mL) were added methanesulfonato(2-dicyclohexylphosphino-2′,4′,6′-tri-i-propyl-1,1′-biphenyl)(2′-amino-1,1′-biphenyl-2-yl)palladium(II) (112 mg, 133 μmol), potassium phosphate (848 mg, 4.0 mmol) and dicyclohexyl(2′,4′,6′-triisopropyl-[1,1′-biphenyl]-2-yl)phosphane (63.5 mg, 133 μmol) at 25° C. under nitrogen atmosphere. The resulting mixture was stirred at 100° C. for 16 hours. Product could be detected by LCMS. The mixture was allowed to cool down to 25° C., and diluted with brine (100 mL), then extracted with ethyl acetate (3×100 mL). The combined organic layers were dried over anhydrous sodium sulfate and filtered. The filtrate was concentrated under reduced pressure. The residue was purified by silica gel column chromatography, eluted with 0%-70% ethyl acetate in petroleum ether to give 5-(4-cyclopropyl-6-methoxypyrimidin-5-yl)-1-((2-(trimethylsilyl)ethoxy)methyl)-1H-pyrazolo[3,4-c]pyridine (350 mg, 880.40 μmol, 66% yield) as a yellow solid. MS: m/z=398.10 [M+H]⁺. ¹H NMR (400 MHz, Chloroform-d) δ 9.46 (s, 1H), 8.67 (s, 1H), 8.26 (s, 1H), 7.70 (s, 1H), 5.84 (s, 2H), 3.95 (s, 3H), 3.77-3.68 (m, 2H), 1.88-1.82 (m, 1H), 1.33-1.20 (m, 2H), 1.05-0.96 (m, 2H), 0.94-0.90 (m, 2H), 0.03 (s, 9H).

### 1.3 5-(4-cyclopropyl-6-methoxypyrimidin-5-yl)-1H-pyrazolo[3,4-c]pyridine

To a solution of 5-(4-cyclopropyl-6-methoxypyrimidin-5-yl)-1-((2-(trimethylsilyl)ethoxy)methyl)-1H-pyrazolo[3,4-c]pyridine (660 mg, 1.66 mmol) in dichloromethane (3 mL) was added trifluoroacetic acid (3 mL) at 25° C. under nitrogen atmosphere. The reaction mixture was stirred at 25° C. for 1 hour and then concentrated under reduced pressure. The residue was dissolved in tetrahydrofuran (3 mL), then ammonium hydroxide (3 mL) was added at 0° C. The reaction mixture was stirred at 25° C. for 1 hour and then concentrated under reduced pressure. The residue was purified by silica gel column chromatography, eluted with 50%-100% ethyl acetate in petroleum ether to give 5-(4-cyclopropyl-6-methoxypyrimidin-5-yl)-1H-pyrazolo[3,4-c]pyridine (380 mg, 1.42 mmol, 86% yield) as yellow oil. MS: m/z=268.05. [M+H]⁺.

### 1.4 3-bromo-5-(4-cyclopropyl-6-methoxypyrimidin-5-yl)-1H-pyrazolo[3,4-c]pyridine

To a solution of 5-(4-cyclopropyl-6-methoxypyrimidin-5-yl)-1H-pyrazolo[3,4-c]pyridine (380 mg, 1.42 mmol) in N,N-dimethylformamide (8 mL) was added N-Bromosuccinimide (380 mg, 2.13 mmol) at 25° C. under nitrogen atmosphere. The reaction mixture was stirred at 25° C. for 3 hours. The resulted mixture was diluted with brine (60 mL), extracted with ethyl acetate (3×60 mL). The combined organic layers were dried over anhydrous sodium sulfate and filtered. The filtrate was concentrated under reduced pressure. The residue was purified by silica gel column chromatography, eluted with 50%-85% ethyl acetate in petroleum ether to give 3-bromo-5-(4-cyclopropyl-6-methoxypyrimidin-5-yl)-1H-pyrazolo[3,4-c]pyridine (400 mg, 1.16 mmol, 81% yield) as a yellow oil. MS: m/z=345.90, 347.85 [M+H]⁺. ¹H NMR (400 MHz, Chloroform-d) δ 9.22 (s, 1H), 8.68 (s, 1H), 8.48 (s, 1H), 7.70 (s, 1H), 3.94 (s, 3H), 1.77-1.69 (m, 1H), 1.28-1.20 (m, 2H), 0.96-0.87 (m, 2H).

### 1.5 3-bromo-5-(4-cyclopropyl-6-methoxypyrimidin-5-yl)-1-((2-(trimethylsilyl)ethoxy)methyl)-1H-pyrazolo[3,4-c]pyridine and 3-bromo-5-(4-cyclopropyl-6-methoxypyrimidin-5-yl)-2-((2-(trimethylsilyl)ethoxy)methyl)-2H-pyrazolo[3,4-c]pyridine

To a solution of 3-bromo-5-(4-cyclopropyl-6-methoxypyrimidin-5-yl)-1H-pyrazolo[3,4-c]pyridine (400 mg, 1.16 mmol) in tetrahydrofuran (8 mL) was added sodium hydride (58 mg, 1.44 mmol, 60% dispersion in oil) at 0° C. under nitrogen atmosphere. The reaction mixture was stirred at 0° C. for 45 min, then 2-(chloromethoxy)ethyl-trimethyl-silane (241 mg, 1.44 mmol) was added to the reaction mixture at 0° C. The reaction was stirred at 25° C. for 16 hours. Then the reaction was quenched with saturated ammonium chloride aqueous solution (40 mL), and then extracted with ethyl acetate (3×50 mL). The combined organic extracts were washed with brine (30 mL), dried over anhydrous sodium sulfate. After filtration, the filtrate was concentrated under reduced pressure. The residue was purified by silica gel column chromatography, eluted with 1%-30% ethyl acetate in petroleum ether to give a mixture of 3-bromo-5-(4-cyclopropyl-6-methoxypyrimidin-5-yl)-1-((2-(trimethylsilyl)ethoxy)methyl)-1H-pyrazolo[3,4-c]pyridine and 3-bromo-5-(4-cyclopropyl-6-methoxypyrimidin-5-yl)-2-((2-(trimethylsilyl)ethoxy)methyl)-2H-pyrazolo[3,4-c]pyridine (330 mg, 692.64 μmol, 60% yield) as an off-white solid. MS: m/z=476.05, 477.95 [M+H]⁺

### 1.6 5-(4-cyclopropyl-6-methoxypyrimidin-5-yl)-3-(4-(1-methyl-4-(trifluoromethyl)−1H-imidazol-2-yl)benzyl)-1-((2trimethylsilyl)ethoxy)methyl)-1H-pyrazolo[3,4-c]pyridine And 5-(4-cyclopropyl-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[3,4-c]pyridine

To a solution of 3-bromo-5-(4-cyclopropyl-6-methoxypyrimidin-5-yl)-1-((2-(trimethylsilyl)ethoxy)methyl)-1H-pyrazolo[3,4-c]pyridine and 3-bromo-5-(4-cyclopropyl-6-methoxypyrimidin-5-yl)-2-((2-(trimethylsilyl)ethoxy)methyl)-2H-pyrazolo[3,4-c]pyridine (270 mg, 567 μmol) in water (1 mL) and toluene (5 mL) were added 1-methyl-2-(4-((4,4,5,5-tetramethyl-1,3,2-dioxaborolan-2-yl)methyl)phenyl)-4-(trifluoromethyl)-1H-imidazole (249 mg, 680 μmol), bis(triphenylphosphine)palladium(II) chloride (119 mg, 170 μmol) and potassium phosphate (241 mg, 1.13 mmol) at 25° C. under nitrogen atmosphere. The reaction mixture was stirred at 90° C. for 16 hours. The reaction progress was monitored by LCMS. The resulting mixture was cooled down to room temperature and the solid was filtered out. The filtrate was concentrated under reduced pressure. The residue was purified by silica gel column chromatography, eluted with 0%-70% ethyl acetate in petroleum ether to give 5-(4-cyclopropyl-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[3,4-c]pyridine and 5-(4-cyclopropyl-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[3,4-c]pyridine (100 mg, 157.29 μmol, 28% yield, mixture of two isomers) as a yellow solid. MS: m/z=636.20 [M+H]⁺.

### 1.7 5-(4-cyclopropyl-6-methoxypyrimidin-5-yl)-3-(4-(1-methyl-4-(trifluoromethyl)-1H-imidazol-2-yl)benzyl)-1H-pyrazolo[3,4-c]pyridine

To a solution of 5-(4-cyclopropyl-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[3,4-c]pyridine and 5-(4-cyclopropyl-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[3,4-c]pyridine (90 mg, 142 μmol) in dichloromethane (2 mL) was added trifluoroacetic acid (2 mL) at 0° C. under nitrogen atmosphere. The reaction mixture was stirred at 25° C. for 1.5 hours and then concentrated under reduced pressure. The residue was dissolved in tetrahydrofuran (2 mL), then ammonium hydroxide (2 mL) was added at 0° C. The reaction mixture was stirred at 25° C. for 1 hour and then concentrated under reduced pressure. The residue was continued purified by Prep-TLC to give a crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 25 mL/min; Gradient: 5% B to 5% B in 3 min, 5% B to 55% B in 8 min, 55% B to 55% B in 3 min, 55% B to 95% B in 8 min; Detector: UV 254 & 210 nm; RT: 12 min. The collected fractions were combined, concentrated and then lyophilized overnight to give 5-(4-cyclopropyl-6-methoxypyrimidin-5-yl)-3-(4-(1-methyl-4-(trifluoromethyl)-1H-imidazol-2-yl)benzyl)-1H-pyrazolo[3,4-c]pyridine (12.8 mg, 25.32 μmol, 18% yield) as an off-white solid. MS: m/z=506.25 [M+H]⁺. ¹H NMR (400 MHz, Chloroform-d) δ 9.39 (s, 1H), 8.65 (s, 1H), 7.63-7.60 (m, 3H), 7.44 (d, J=8.0 Hz, 2H), 7.33 (s, 1H), 4.49 (s, 2H), 3.90 (s, 3H), 3.77 (s, 3H), 1.71-1.63 (m, 1H), 1.25-1.19 (m, 2H), 0.90-0.84 (m, 2H). ¹⁹F NMR (376 MHz, Chloroform-d) δ −62.69.

### Example 73. 5-[4-cyclopropyl-6-(difluoromethoxy)pyrimidin-5-yl]-3-[[4-[1-methyl-4-(trifluoromethyl)imidazol-2-yl]phenyl]methyl]-1H-pyrazolo[4,3-d]pyrimidine Compound 185

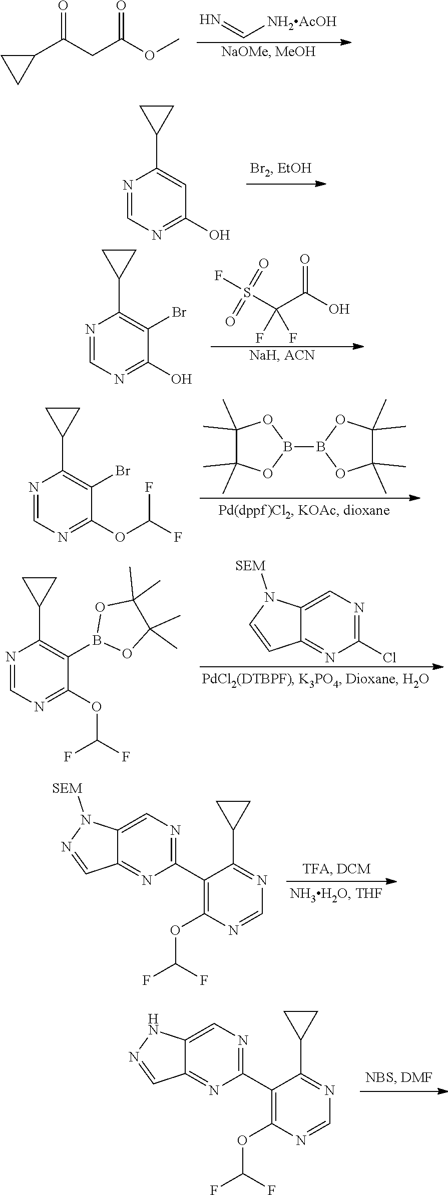

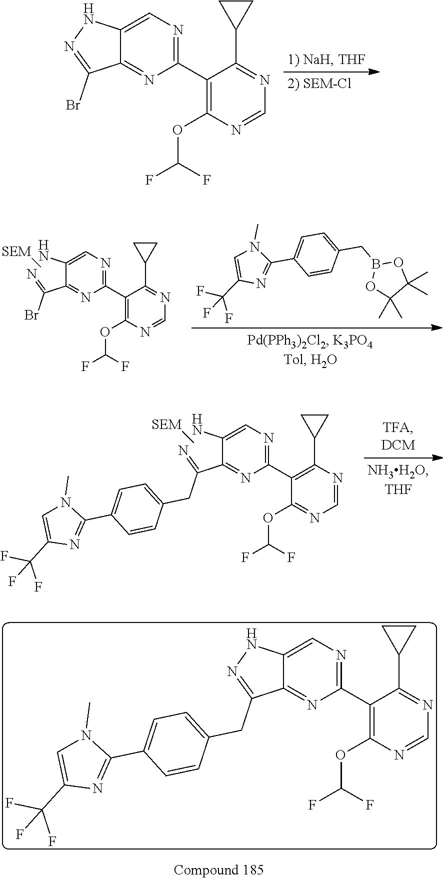

### 1.1 6-cyclopropylpyrimidin-4-ol

To a solution of methyl 3-cyclopropyl-3-oxo-propanoate (10 g, 70.35 mmol) and formimidamide acetate (6.20 g, 140.69 mmol) in methanol (250 mL) was added sodium methanolate (26.03 g, 481.88 mmol) at 25° C. under nitrogen atmosphere. The reaction mixture was stirred at 25° C. for 72 hours. The resulted mixture was acidified to pH=7 with acetic acid and filtered. The filtrate was concentrated under reduced pressure. The residue was purified by silica gel column chromatography, eluted with 4%-5% methanol in dichloromethane to afford 6-cyclopropylpyrimidin-4-ol (5 g, 36.72 mmol, 52% yield) as a yellow solid. ¹H NMR (400 MHz, Methanol-d₄) δ 8.04 (s, 1H), 6.33 (s, 1H), 1.93-1.87 (m, 1H), 1.07-0.99 (m, 4H).

### 1.2 5-bromo-6-cyclopropyl-pyrimidin-4-ol

To a solution of 6-cyclopropylpyrimidin-4-ol (2.5 g, 18.36 mmol) in ethanol (250 mL) was added bromine (5.87 g, 36.72 mmol) at 25° C. under nitrogen atmosphere, and the reaction mixture was stirred at 25° C. for 16 hours. The reaction was concentrated under reduced pressure to yield a crude product. The residue was purified by silica gel column chromatography, eluted with 4%-5% methanol in dichloromethane to give 5-bromo-6-cyclopropyl-pyrimidin-4-ol (3.6 g, 16.74 mmol, 91% yield) as a light-yellow solid. MS: m/z=215.00, 217.00 [M+H]⁺. ¹H NMR (400 MHz, Methanol-d₄) δ 7.98 (s, 1H), 2.55-2.47 (m, 1H), 1.16-1.12 (m, 2H), 1.11-1.07 (m, 2H).

### 1.3 5-bromo-5-cyclopropyl-6-(difluoromethoxy)pyrimidine

To a solution of 5-bromo-6-cyclopropyl-pyrimidin-4-01 (3.6 g, 16.74 mmol) in acetonitrile (300 mL) was added sodium hydride (1.21 g, 50.22 mmol, 60% dispersion in mineral oil) at 25° C. under nitrogen atmosphere, and the suspension was stirred at 25° C. for 45 min. Then 2,2-difluoro-2-fluorosulfonyl-acetic acid (5.07 g, 28.46 mmol, 2.94 mL) was added to the reaction mixture and stirred at 25° C. for 2 hours. The reaction was quenched with saturated ammonium chloride aqueous solution (500 mL), extracted with ethyl acetate (3×500 mL). The combined organic extracts were washed with brine (200 mL), dried over anhydrous sodium sulfate. After filtration, the filtrate was concentrated under reduced pressure to yield a crude product. The residue was purified by silica gel column chromatography, eluted with 10%-30% ethyl acetate in petroleum ether to give 5-bromo-4-cyclopropyl-6-(difluoromethoxy)pyrimidine (2.8 g, 10.56 mmol, 63% yield) as an off-white solid. MS: m/z=265.00, 267.00 [M+H]⁺. ¹H NMR (300 MHz, Chloroform-d) δ 8.49 (s, 1H), 7.50 (t, J=73.2 Hz, 1H), 2.65-2.56 (m, 1H), 1.32-1.19 (m, 4H).

### 1.4 4-cyclopropyl-6-(difluoromethoxy)-5-(4,4,5,5-tetramethyl-1,3,2-dioxaborolan-2-yl)pyrimidine

To a solution of 5-bromo-4-cyclopropyl-6-(difluoromethoxy)pyrimidine (1 g, 3.77 mmol) and 4,4,5,5-tetramethyl-2-(4,4,5,5-tetramethyl-1,3,2-dioxaborolan-2-yl)-1,3,2-dioxaborolane (958 mg, 3.77 mmol) in 1,4-dioxane (15 mL) was added 1,1′-bis(diphenylphosphino)ferrocene palladium(II)dichloride (308 mg, 377 μmol) and potassium acetate (741 mg, 7.6 mmol) at 25° C. under nitrogen atmosphere. The mixture was heated up to 95° C., and stirred for 16 hours under nitrogen atmosphere. The reaction was detected by TLC. The mixture was cooled down to 25° C. The mixture was diluted with ethyl acetate (300 mL) and washed with brine (4×100 mL), dried over anhydrous sodium sulfate. After filtration, the filtrate was concentrated under reduced pressure. The residue was purified by silica gel column chromatography, eluted with 10%-20% ethyl acetate in petroleum ether to afford 4-cyclopropyl-6-(difluoromethoxy)-5-(4,4,5,5-tetramethyl-1,3,2-dioxaborolan-2-yl)pyrimidine (350 mg, 1.12 mmol, 30% yield) as a white solid. MS: m/z=313.00 [M+H]⁺. ¹H NMR (400 MHz, Chloroform-d) δ 8.64 (s, 1H), 7.46 (t, J=73.2 Hz, 1H), 2.26-2.18 (m, 1H), 1.43 (s, 12H), 1.34-1.30 (m, 2H), 1.15-1.11 (m, 2H).

### 1.5 2-[[5-[4-cyclopropyl-6-(difluoromethoxy)pyrimidin-5-yl]pyrazolo[4,3-d]pyrimidin-1-yl]methoxy]ethyl-trimethyl-silane

To a solution of 4-cyclopropyl-6-(difluoromethoxy)-5-(4,4,5,5-tetramethyl-1,3,2-dioxaborolan-2-yl)pyrimidine (300 mg, 961.17 μmol) and 2-[(5-chloropyrazolo[4,3-d]pyrimidin-1-yl)methoxy]ethyl-trimethyl-silane (274 mg, 961 μmol) in 1,4-dioxane (5 mL) and water (1 mL) were added 1,1′-Bis (di-t-butylphosphino)ferrocene palladium dichloride (63 mg, 96 μmol) and potassium phosphate (408 mg, 1.9 mmol) at 25° C. under nitrogen atmosphere. The mixture was heated up to 100° C., and stirred for 16 hours under nitrogen atmosphere. The reaction was detected by TLC. The mixture was cooled down to 25° C. The resulted mixture was diluted with ethyl acetate (300 mL) and washed with brine (3×140 mL), dried over anhydrous sodium sulfate. After filtration, the filtrate was concentrated under reduced pressure. The residue was purified by silica gel column chromatography, eluted with 60%-70% ethyl acetate in petroleum ether to give 2-[[5-[4-cyclopropyl-6-(difluoromethoxy)pyrimidin-5-yl]pyrazolo[4,3-d]pyrimidin-1-yl]methoxy]ethyl-trimethyl-silane (300 mg, 690.43 μmol, 72% yield) as a yellow oil. MS: m/z =435.15 [M+H]⁺. ¹H NMR (400 MHz, Chloroform-d) δ 9.44 (s, 1H), 8.72 (s, 1H), 8.37 (s, 1H), 7.52 (t, J=73.2 Hz, 1H), 5.90 (s, 2H), 3.72-3.60 (m, 2H), 1.85-1.77 (m, 11H), 1.38-1.28 (m, 2H), 1.08-0.86 (m, 4H).

### 1.6 5-[4-cyclopropyl-6-(difluoromethoxy)pyrimidin-5-yl]-1H-pyrazolo[4,3-d]pyrimidine

To a solution of 2-[[5-[4-cyclopropyl-6-(difluoromethoxy)pyrimidin-5-yl]pyrazolo[4,3-d]pyrimidin-1-yl]methoxy]ethyl-trimethyl-silane (300 mg, 690.43 μmol) in dichloromethane (5 mL) was added trifluoroacetic acid (7.40 g, 64.90 mmol, 5 mL) at 0° C. under nitrogen atmosphere. The reaction mixture was stirred at 25° C. for 2 hours. The reaction solution was concentrated under vacuum. The residue was added ammonium hydroxide (28% NH₃ in H₂O, 5 mL) and tetrahydrofuran (5 mL). The resulted mixture was stirred at 25° C. for 30 minutes. The reaction solution was concentrated under vacuum. The residue was purified by silica gel column chromatography, eluted with 4%-5% methanol in dichloromethane to give 5-[4-cyclopropyl-6-(difluoromethoxy)pyrimidin-5-yl]-1H-pyrazolo[4,3-d]pyrimidine (200 mg, 657.34 μmol, 95% yield) as a yellow solid. MS: m/z=304.95 [M+H]⁺. ¹H NMR (400 MHz, Chloroform-d) δ 9.34 (s, 1H), 8.71 (s, 1H), 8.46 (s, 1H), 7.52 (t, J=73.2 Hz, 1H), 1.82-1.79 (m, 1H), 1.36-1.28 (m, 2H), 1.04-0.98 (m, 2H).

### 1.7 3-bromo-5-[4-cyclopropyl-6-(difluoromethoxy)pyrimidin-5-yl]-1H-pyrazolo[4,3-d]pyrimidine

To a solution of 5-[4-cyclopropyl-6-(difluoromethoxy)pyrimidin-5-yl]-1H-pyrazolo[4,3-d]pyrimidine (190 mg, 624 μmol) in N,N-dimethylformamide (4 mL) was added 1-bromopyrrolidine-2,5-dione (167 mg, 934 μmol) at 25° C. under nitrogen atmosphere. The resulting mixture was stirred at 25° C. for 3 hours. The resulted mixture was diluted with ethyl acetate (150 mL), washed with brine (3×150 mL), dried over anhydrous sodium sulfate. After filtration, the filtrate was concentrated under reduced pressure. The residue was purified by silica gel column chromatography, eluted with 4%-5% methanol in dichloromethane to give 3-bromo-5-[4-cyclopropyl-6-(difluoromethoxy)pyrimidin-5-yl]-1H-pyrazolo[4,3-d]pyrimidine (200 mg, 521.99 μmol, 84% yield) as a yellow solid MS: m/z=382.95, 384.95 [M+H]⁺.

### 1.8 3-bromo-5-(4-cyclopropyl-6-(difluoromethoxy)pyrimidin-5-yl)-1-((2-(trimethylsilyl)ethoxy)methyl)-1H-pyrazolo[4,3-d]pyrimidine and 3-bromo-5-(4-cyclopropyl-6-(difluoromethoxy)pyrimidin-5-yl)-2-((2-(trimethylsilyl)ethoxy)methyl)-2H-pyrazolo[4,3-d]pyrimidine

To a solution of 3-bromo-5-[4-cyclopropyl-6-(difluoromethoxy)pyrimidin-5-yl]-1H-pyrazolo[4,3-d]pyrimidine (200 mg, 521.99 μmol) in tetrahydrofuran (3 mL) was added sodium hydride (15 mg, 626 μmol, 60% dispersion in mineral oil) at 0° C. under nitrogen atmosphere. The reaction suspension was stirred at 0° C. for 45 minutes. Then 2-(chloromethoxy)ethyl-trimethyl-silane (104 mg, 626 μmol, 111 μL) was added to the reaction suspension and stirred at 25° C. for 2 hours. The reaction was quenched with saturated ammonium chloride aqueous solution (100 mL), extracted with ethyl acetate (3×100 mL). The combined organic extracts were washed with brine (100 mL), dried over anhydrous sodium sulfate. After filtration, the filtrate was concentrated under reduced pressure to yield a crude product. The residue was purified by silica gel column chromatography, eluted with 30%-35% ethyl acetate in petroleum ether to give a mixture of 3-bromo-5-(4-cyclopropyl-6-(difluoromethoxy)pyrimidin-5-yl)-1-((2-(trimethylsilyl)ethoxy)methyl)-1H-pyrazolo[4,3-d]pyrimidine and 3-bromo-5-(4-cyclopropyl-6-(difluoromethoxy)pyrimidin-5-yl)-2-((2-(trimethylsilyl)ethoxy)methyl)-2H-pyrazolo[4,3-d]pyrimidine (260 mg, 506.42 μmol, 97% yield) as a light-yellow oil. MS: m/z=513.10, 515.10 [M+H]⁺.

### 1.9 5-(4-cyclopropyl-6-(difluoromethoxy)pyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine and 5-(4-cyclopropyl-6-(difluoromethoxy)pyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine

To a solution of 3-bromo-5-(4-cyclopropyl-6-(difluoromethoxy)pyrimidin-5-yl)-1-((2-(trimethylsilyl)ethoxy)methyl)-1H-pyrazolo[4,3-d]pyrimidine and 3-bromo-5-(4-cyclopropyl-6-(difluoromethoxy)pyrimidin-5-yl)-2-((2-(trimethylsilyl)ethoxy)methyl)-2H-pyrazolo[4,3-d]pyrimidine (150.00 mg, 292.16 μmol) in toluene (2.5 mL) and H₂O (0.5 mL) were added 1-methyl-2-[4-[(4,4,5,5-tetramethyl-1,3,2-dioxaborolan-2-yl)methyl]phenyl]-4-(trifluoromethyl)imidazole (107 mg, 292 μmol), bis(triphenylphosphine)palladium(II) chloride (61.5 mg, 88 μmol) and potassium phosphate (186 mg, 876 μmol) at 25° C. under nitrogen atmosphere. The resulted suspension was heated up to 90° C., and stirred for 16 hours under nitrogen atmosphere. The resulted mixture was cooled down to 25° C. The resulted mixture was diluted with ethyl acetate (200 mL) and washed with brine (3×100 mL), dried over anhydrous sodium sulfate. After filtration, the filtrate was concentrated under reduced pressure. The residue was purified by silica gel column chromatography, eluted with 60%-70% ethyl acetate in petroleum ether to give a yellow mixture of 5-(4-cyclopropyl-6-(difluoromethoxy)pyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine and 5-(4-cyclopropyl-6-(difluoromethoxy)pyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (90 mg, 133.78 μmol, 45% yield) as a solid. MS: m/z=673.30 [M+H]⁺.

### 1.10 5-[4-cyclopropyl-6-(difluoromethoxy)pyrimidin-5-yl]-3-[[4-[1-methyl-4-(trifluoromethyl)imidazol-2-yl]phenyl]methyl]-1H-pyrazolo[4,3-d]pyrimidine

To a solution of 5-(4-cyclopropyl-6-(difluoromethoxy)pyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine and 5-(4-cyclopropyl-6-(difluoromethoxy)pyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (90 mg, 133.78 μmol) in dichloromethane (3 mL) was added trifluoroacetic acid (4.44 g, 38.94 mmol, 3 mL) at 0° C. under nitrogen atmosphere. The reaction mixture was stirred at 25° C. for 2 hours. The resulted reaction solution was concentrated under reduce pressure. The residue was added ammonium hydroxide (28% NH₃ in H₂O, 3 mL) and tetrahydrofuran (3 mL). The resulting mixture was stirred at 25° C. for 30 minutes. The reaction solution was concentrated under vacuum. The residue was purified by silica gel column chromatography, eluted with 3%-5% methyl alcohol in dichloromethane to afford a crude product. Then, the crude product was further purified by RP-Flash chromatography with the following conditions: Column: C18 spherical Column, 20-35 um, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 43% B in 10 min, 43% B to 43% B in 5 min, 43% B to 95% B in 10 min; Detector: UV 254 & 210 nm; RT: 20 min. The fractions of first eluting peak (RT: 20 min) were collected, concentrated under reduced pressure and then lyophilized overnight to afford 5-[4-cyclopropyl-6-(difluoromethoxy)pyrimidin-5-yl]-3-[[4-[1-methyl-4-(trifluoromethyl)imidazol-2-yl]phenyl]methyl]-1H-pyrazolo[4,3-d]pyrimidine (15 mg, 27.65 μmol, 21% yield) as a light-yellow solid. MS: m/z=543.35 [M+H]⁺. ¹H NMR (400 MHz, Methanol-d₄) δ 9.39 (s, 1H), 8.73 (s, 1H), 7.67 (t, J=73.2 Hz, 1H), 7.66 (s, 1H), 7.61-7.55 (m, 4H), 4.56 (s, 2H), 3.75 (s, 3H), 1.81-1.74 (m, 1H), 1.28-1.22 (m, 2H), 0.99-0.92 (m, 2H). ¹⁹F NMR (376 MHz, Methanol-d₄) δ −63.94, −91.24.

### Example 74. 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine Compound 195

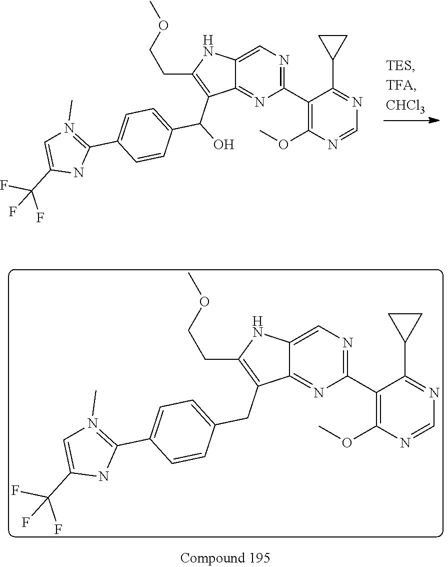

### 1.1 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine

A mixture of [2-(4-cyclopropyl-6-methoxy-pyrimidin-5-yl)-6-(2-methoxyethyl)-5H-pyrrolo[3,2-d]pyrimidin-7-yl]-[4-[1-methyl-4-(trifluoromethyl)imidazol-2-yl]phenyl]methanol (40 mg, 69 μmol) in trifluoroacetic acid (2 mL), triethylsilane (4 mL) and chloroform (4 mL) was stirred at room temperature for 16 hours. The residue was purified by Prep-TLC (dichloromethane:methanol=15:1) to afford crude product as a light yellow oil. The crude was further purified by reverse phase chromatography with the following conditions: Column: Spherical C18, 20-40 um, 20 g; Mobile Phase A: Water (5 mM aq. NH₄HCO₃), Mobile Phase B: ACN; Flow rate: 30 mL/min; Gradient (B %): 5% hold 5 min, 5%-42% within 12 min, 42% hold 2 min; 42%-95% within 10 min, 95% hold 5 min; Detector: UV 254 & 210 nm; RT: 17 min. The collected fractions were combined and concentrated under reduced pressure to afford 2-(4-cyclopropyl-6-methoxy-pyrimidin-5-yl)-6-(2-methoxyethyl)-7-[[4-[-methyl-4-(trifluoromethyl)imidazol-2-yl]phenyl]methyl]-5H-pyrrolo[3,2-d]pyrimidine (30 mg, 53.23 μmol, 77% yield) as an off-white solid. MS: m/z=564.30 [M+H]⁺. ¹H NMR (400 MHz, Chloroform-d) δ 9.57 (s, 1H), 8.97 (s, 1H), 8.67 (s, 1H), 7.53-7.45 (m, 2H), 7.38 (d, J=8.0 Hz, 2H), 7.31 (s, 1H), 4.28 (s, 2H), 3.95 (s, 3H), 3.74 (s, 3H), 3.70-3.63 (m, 2H), 3.45 (s, 3H), 3.04 (t, J=5.6 Hz, 2H), 1.82-1.71 (m, 1H), 1.30-1.18 (m, 2H), 0.90-0.80 (m, 2H). ¹⁹F NMR (376 MHz, Chloroform-d) δ −62.71.

### Example 75. 5-(4-cyclopropyl-6-methoxy-pyrimidin-5-yl)-3-[[4-[1-methyl-4-(trifluoromethyl)imidazol-2-yl]phenyl]methyl]-1-(trideuteriomethyl)pyrazolo[4,3-d]pyrimidine (Compound 143) & 5-(4-cyclopropyl-6-methoxy-pyrimidin-5-yl)-3-[[4-[1-methyl-4-(trifluoromethyl)imidazol-2-yl]phenyl]methyl]-2-(trideuteriomethyl)pyrazolo[4,3-d]pyrimidine

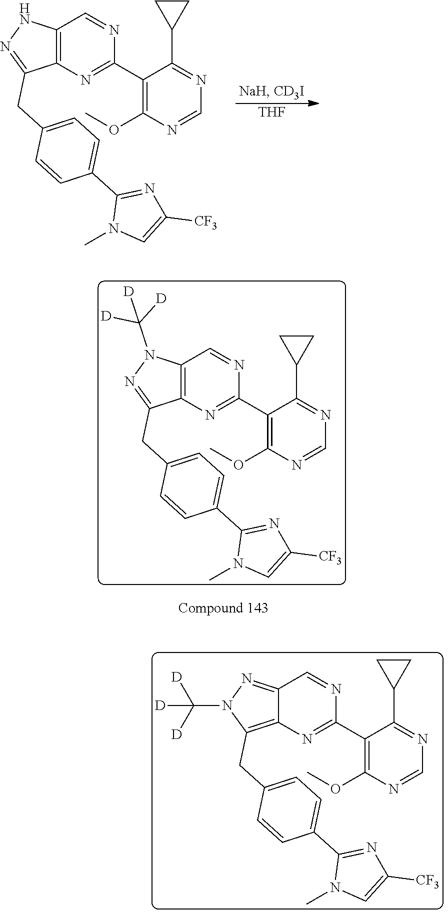

### 1.1 5-(4-cyclopropyl-6-methoxy-pyrimidin-5-yl)-3-[[4-[1-methyl-4-(trifluoromethyl)imidazol-2-yl]phenyl]methyl]-1-(trideuteriomethyl)pyrazolo[4,3-d]pyrimidine (Compound 143) & 5-(4-cyclopropyl-6-methoxy-pyrimidin-5-yl)-3-[[4-[1-methyl-4-(trifluoromethyl)imidazol-2-yl]phenyl]methyl]-2-(trideuteriomethyl)pyrazolo[4,3-d]pyrimidine

To a solution of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (230 mg, 454 μmol) in tetrahydrofuran (3 mL) under nitrogen atmosphere was added sodium hydride (27 mg, 681 μmol, 60% dispersion in oil) at 0° C. The resulting mixture was stirred at 0° C. for 1 hour. To the above mixture was added trideuterio(iodo)methane (79 mg, 545 μmol, 34 μL) at 0° C. The resulting mixture was stirred at 25° C. for 2 hours. The reaction was monitored by LCMS. The resulted mixture was quenched by saturated chloride aqueous ammonium (30 mL), extracted with ethyl acetate (3×30 mL). The combined organic layers were washed with brine (20 mL), dried over anhydrous anhydrous sodium sulfate. After filtration, the filtrate was concentrated under reduced pressure. The residue was purified by Prep-HPLC with the following conditions: Column: XBridge Shield RP18 OBD Column, 30×150 mm, 5 μm; Mobile Phase A: Water (0.1% FA), Mobile Phase B: MeCN; Flow rate: 60 mL/min; Gradient: 35% B to 50% B in 12 min; Wave Length: 220 nm; RT1: 6.03 min; RT2: 7.78 min. The product containing (RT1: 6.03 min) peak was combined, concentrated under reduced pressure and then lyophilized to give 5-(4-cyclopropyl-6-methoxy-pyrimidin-5-yl)-3-[[4-[1-methyl-4-(trifluoromethyl)imidazol-2-yl]phenyl]methyl]-1-(trideuteriomethyl)pyrazolo[4,3-d]pyrimidine (66.5 mg, 127.02 μmol, 28% yield) as an off-white solid. MS: m/z=524.35 [M+H]⁺. ¹H NMR (400 MHz, Chloroform-d) δ 9.23 (s, 1H), 8.71 (s, 1H), 7.59-7.53 (m, 4H), 7.31 (s, 1H), 4.52 (s, 2H), 3.95 (s, 3H), 3.75 (s, 3H), 1.70-1.64 (m, 1H), 1.29-1.26 (m, 2H), 0.91-0.86 (m, 2H). ¹⁹F NMR (376 MHz, Chloroform-d) δ −62.74.

The product containing (RT2: 7.78 min) peak was combined, concentrated under reduced pressure and then lyophilized to give 5-(4-cyclopropyl-6-methoxy-pyrimidin-5-yl)-3-[[4-[1-methyl-4-(trifluoromethyl)imidazol-2-yl]phenyl]methyl]-2-(trideuteriomethyl)pyrazolo[4,3-d]pyrimidine (34.3 mg, 65.52 μmol, 14% yield) as an off-white solid. MS: m/z=524.30 [M+H]⁺. ¹H NMR (400 MHz, Chloroform-d) δ 9.55 (s, 1H), 8.70 (s, 1H), 7.59 (d, J=8.0 Hz, 2H), 7.35-7.32 (m, 3H), 4.64 (s, 2H), 3.97 (s, 3H), 3.76 (s, 3H), 1.79-1.73 (m, 1H), 1.32-1.24 (m, 2H), 0.95-0.90 (m, 2H). ¹⁹F NMR (376 MHz, Chloroform-d) δ −62.73.

### Example 76. 5-(4-cyclopropyl-6-methoxypyrimidin-5-yl)-7-methyl-3-(4-(1-methyl-4-(trifluoromethyl)-1H-imidazol-2-yl)benzyl)-1H-pyrazolo[4,3-d]pyrimidine Compound 115

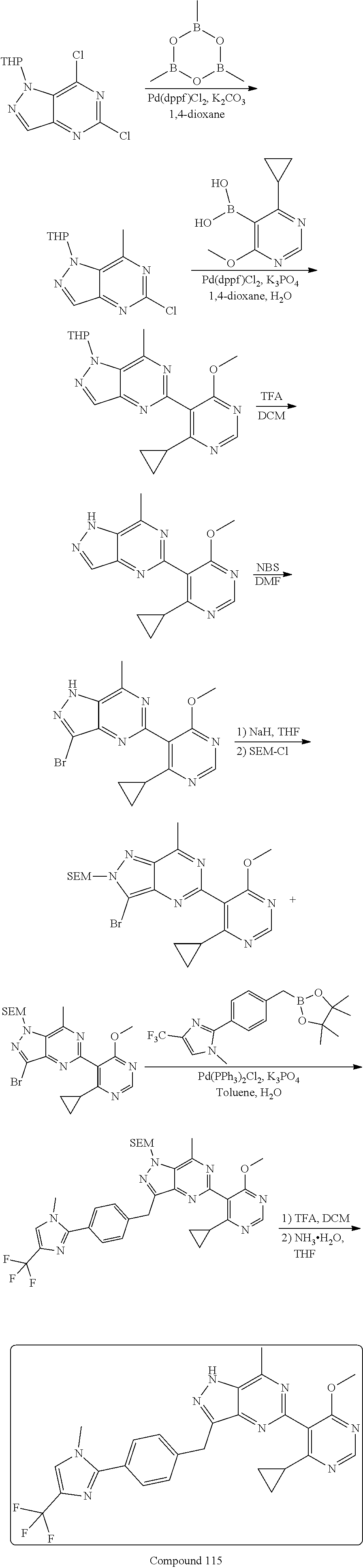

### 1.1 5-chloro-7-methyl-1-(tetrahydro-2H-pyran-2-yl)-1H-pyrazolo[4,3-d]pyrimidine

To a solution of 5,7-dichloro-1-(tetrahydro-2H-pyran-2-yl)-1H-pyrazolo[4,3-d]pyrimidine (700 mg, 2.56 mmol) and 2,4,6-trimethyl-1,3,5,2,4,6-trioxatriborinane (708 mg, 5.13 mmol, 50% in THF) in 1,4-dioxane (10 mL) were added 1,1′-Bis(diphenylphosphino)ferrocene-palladium(II)dichloride (209 mg, 256 μmol) and potassium carbonate (708 mg, 5.13 mmol) at 25° C. The resulting mixture was stirred for 4 hours at 100° C. then cooled down to rt. The residue was diluted with ethyl acetate (50 mL). The solid was filtered out and the filtrate was concentrated under vacuum. The residue was purified by silica gel column chromatography, eluted with 35%-45% EtOAc in PE to afford 5-chloro-7-methyl-1-(tetrahydro-2H-pyran-2-yl)-1H-pyrazolo[4,3-d]pyrimidine (540 mg, 2.14 mmol, 84% yield) as a yellow solid. MS: m/z=252.95 [M+H]⁺. ¹H NMR (400 MHz, Chloroform-d) δ 8.32 (s, 1H), 5.75-5.72 (m, 1H), 4.19-4.15 (m, 1H), 3.86-3.80 (m, 1H), 2.92 (s, 3H), 2.32-2.30 (m, 1H), 2.20-2.04 (m, 2H), 1.85-1.69 (m, 3H).

### 1.2 5-(4-cyclopropyl-6-methoxypyrimidin-5-yl)-7-methyl-1-(tetrahydro-2H-pyran-2-yl)-1H-pyrazolo[4,3-d]pyrimidine

To a solution of 5-chloro-7-methyl-1-(tetrahydro-2H-pyran-2-yl)-1H-pyrazolo[4,3-d]pyrimidine (540 mg, 2.14 mmol) and (4-cyclopropyl-6-methoxy-pyrimidin-5-yl)boronic acid (415 mg, 2.14 mmol) in 1,4-dioxane (5 mL) and H₂O (1 mL) added 1,1′-Bis(diphenylphosphino)ferrocene-palladium(II)dichloride (174.5 mg, 214 μmol) and potassium phosphate (1.36 g, 6.41 mmol) at 25° C. The resulting mixture was stirred for 16 hours at 100° C. and cooled down to room temperature. The resulting mixture was filtered through a Celite pad.

The filtrate was concentrated under reduced pressure. The residue was purified by silica gel column chromatography, eluted with 60%-80% EtOAc in PE to afford 5-(4-cyclopropyl-6-methoxypyrimidin-5-yl)-7-methyl-1-(tetrahydro-2H-pyran-2-yl)-1H-pyrazolo[4,3-d]pyrimidine (400 mg, 1.09 mmol, 51% yield) as a yellow solid. Together 670 mg of product (1.83 mmol) from combined two batches was obtained. MS: m/z=367.20 [M+H]⁺. ¹H NMR (400 MHz, Chloroform-d) δ 8.68 (s, 1H), 8.46 (s, 1H), 5.81-5.78 (m, 1H), 5.32 (s, 2H), 3.94 (s, 3H), 2.99 (s, 3H), 2.34-2.15 (m, 2H), 1.88-1.70 (m, 5H), 1.28-1.25 (m, 2H), 0.95-0.88 (m, 2H).

### 1.3 5-(4-cyclopropyl-6-methoxypyrimidin-5-yl)-7-methyl-1H-pyrazolo[4,3-d]pyrimidine

To a solution of 5-(4-cyclopropyl-6-methoxypyrimidin-5-yl)-7-methyl-1-(tetrahydro-2H-pyran-2-yl)-1H-pyrazolo[4,3-d] (670 mg, 2.37 mmol) in DCM (2 mL) was added trifluoroacetic acid (2 mL). The reaction was stirred for 4 hours at 25° C. then concentrated under reduced pressure. The residue was purified by silica gel column chromatography, eluted with 0%-10% methanol in dichloromethane to afford 5-(4-cyclopropyl-6-methoxy-pyrimidin-5-yl)-7-methyl-1H-pyrazolo[4,3-d]pyrimidine (350 mg, 1.24 mmol, 68% yield) as a yellow solid. MS: m/z=283.20 [M+H]⁺. ¹H NMR (400 MHz, Chloroform-d) δ 8.75 (s, 1H), 8.42 (s, 1H), 3.95 (s, 3H), 2.97 (s, 3H), 1.67-1.65 (m, 1H), 1.31-1.28 (m, 2H), 0.98-0.89 (m, 2H).

### 1.4 3-bromo-5-(4-cyclopropyl-6-methoxypyrimidin-5-yl)-7-methyl-1H-pyrazolo[4,3-d]pyrimidine

To a solution of 5-(4-cyclopropyl-6-methoxy-pyrimidin-5-yl)-7-methyl-1H-pyrazolo[4,3-d]pyrimidine (350 mg, 1.24 mmol) in DMF (7 mL) was added 1-bromopyrrolidine-2,5-dione (199 mg, 1.12 mmol) at 25° C., the resulted solution was stirred for 2 hours at 25° C. After solvent was evaporated, the residue was quenched with water (100 mL), extracted with ethyl acetate (2×50 mL). The combined organic layers were washed with brine (2×50 mL), dried over anhydrous sodium sulfate. After filtration, the filtrate was concentrated under reduced pressure. The residue was purified by Prep-TLC (PE:EA=1:2) to afford crude product, then the crude product was further purified by Prep-TLC (PE:EA=1:10) to afford 3-bromo-5-(4-cyclopropyl-6-methoxy-pyrimidin-5-yl)-7-methyl-1H-pyrazolo[4,3-d]pyrimidine (245 mg, 678.30 μmol, 55% yield) as a brown oil. MS: m/z=361.10, 363.10 [M+H]⁺. ¹H NMR (400 MHz, Chloroform-d) 12.04 (br., 1H), 8.68 (s, 1H), 3.93 (s, 3H), 2.80 (s, 3H), 1.29-1.28 (m, 1H), 1.27-1.25 (m, 2H), 0.95-0.92 (m, 2H).

### 1.5 3-bromo-5-(4-cyclopropyl-6-methoxypyrimidin-5-yl)-7-methyl-1-((2-(trimethylsilyl)ethoxy)methyl)-1H-pyrazolo[4,3-d]pyrimidine & 3-bromo-5-(4-cyclopropyl-6-methoxypyrimidin-5-yl)-7-methyl-2-((2-(trimethylsilyl)ethoxy)methyl)-2H-pyrazolo[4,3-d]pyrimidine

To a solution of 3-bromo-5-(4-cyclopropyl-6-methoxy-pyrimidin-5-yl)-7-methyl-1H-pyrazolo[4,3-d]pyrimidine (245 mg, 678 μmol) in tetrahydrofuran (4 mL) was added sodium hydride (33.5 mg, 814 μmol, 60% in mineral oil) at 0° C. After the resulted mixture was stirred for 1 hour at 25° C., 2-(chloromethoxy)ethyl-trimethyl-silane (124 mg, 746 μmol) was added to the above mixture at 25° C. The resulting mixture was stirred for 2 hours at 25° C., then quenched by saturated ammonium chloride aqueous solution (30 mL), extracted by ethyl acetate (2×30 mL). The organic layer was washed with brine (2×30 mL), dried over anhydrous sodium sulfate. After filtration, the filtrate was concentrated under reduced pressure. The residue was purified by Prep-TLC (PE:EA=10:1) to give 3-bromo-5-(4-cyclopropyl-6-methoxypyrimidin-5-yl)-7-methyl-1-((2-(trimethylsilyl)ethoxy)methyl)-1H-pyrazolo[4,3-d]pyrimidine (100 mg, 204.1 μmol, 30% yield, structure tentatively assigned) as a yellow solid and 3-bromo-5-(4-cyclopropyl-6-methoxypyrimidin-5-yl)-7-methyl-2-((2-(trimethylsilyl)ethoxy)methyl)-2H-pyrazolo[4,3-d]pyrimidine (110 mg, 224.5 μmol, 33% yield, structure tentatively assigned) as a yellow solid. MS: m/z=491.10, 493.10 [M+H]⁺. ¹H NMR (400 MHz, Chloroform-d) δ 8.68 (s, 1H), 5.89 (s, 2H), 3.93 (s, 3H), 3.69-3.65 (m, 2H), 3.10 (s, 3H), 1.60-1.58 (m, 1H), 1.28-1.25 (m, 2H), 0.98-0.94 (m, 2H), 0.93-0.89 (m, 2H), −0.01 (s, 9H).

### 1.6 5-(4-cyclopropyl-6-methoxypyrimidin-5-yl)-7-methyl-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine

To a solution of 3-bromo-5-(4-cyclopropyl-6-methoxypyrimidin-5-yl)-7-methyl-1-((2-(trimethylsilyl)ethoxy)methyl)-1H-pyrazolo[4,3-d]pyrimidine (100 mg, 204.1 μmol) and 1-methyl-2-[4-[(4,4,5,5-tetramethyl-1,3,2-dioxaborolan-2-yl)methyl]phenyl]-4-(trifluoromethyl)imidazole (89 mg, 244 μmol) in toluene (2 mL) and H₂O (0.4 mL) were added Bis(triphenylphosphine)palladium(II) chloride (14 mg, 20 μmol) and potassium phosphate (86 mg, 407 μmol) at 25° C. The resulted mixture was stirred for 16 hours at 25° C. The mixture was filtered through a Celite pad. The filtrate was concentrated under reduced pressure. The residue was purified by Prep-TLC (PE:EA=1:2) to afford 5-(4-cyclopropyl-6-methoxypyrimidin-5-yl)-7-methyl-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine (60 mg, 61.47 μmol, 30% yield) as a yellow solid. MS: m/z=651.50 [M+H]⁺. ¹H NMR (400 MHz, Chloroform-d) 8.82 (s, 1H), 7.73-7.68 (m, 2H), 7.51-7.49 (m, 2H), 7.31 (s, 1H), 5.87 (s, 2H), 4.50 (s, 2H), 3.99 (s, 3H), 3.75 (s, 3H), 3.63 (t, J=8.4 Hz, 2H), 3.07 (s, 3H), 1.76-1.72 (m, 1H), 1.30-1.27 (m, 2H), 0.99-0.86 (m, 4H), −0.01 (s, 9H)

### 1.7 5-(4-cyclopropyl-6-methoxypyrimidin-5-yl)-7-methyl-3-(4-(1-methyl-4-(trifluoromethyl)-1H-imidazol-2-yl)benzyl)-1H-pyrazolo[4,3-d]pyrimidine

The solution of 5-(4-cyclopropyl-6-methoxypyrimidin-5-yl)-7-methyl-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine (60 mg, 61.47 μmol) in DCM (1 mL) and trifluoroacetic acid (1 mL) was stirred for 4 hours at 25° C., then the resulted solution was concentrated under reduced pressure. The residue was added THF (1 mL) and ammonium hydroxide (1 mL, 28% NH₃ in H₂O) and stirred for 10 min. The resulted mixture was concentrated under reduced pressure. The residue was purified by Prep-TLC (DCM:MeOH=10:1) to afford a crude product. The crude product was further purified by RP-Flash chromatography with the following conditions: Column: C18 spherical, 20-30 um, 100A, 40 g; Mobile Phase A: 5 mM aq. NH₄HCO₃. Mobile Phase B: MeCN; Flow rate: 65 mL/min; Gradient: 5% B to 5% B in 5 min, 5% B to 49% B in 20 min; 49% B to 49% B in 5 min; 49% B to 95% B in 5 min; Detector: UV 254 & 220 nm; RT: 28 min to afford 5-(4-cyclopropyl-6-methoxypyrimidin-5-yl)-7-methyl-3-(4-(1-methyl-4-(trifluoromethyl)-1H-imidazol-2-yl)benzyl)-1H-pyrazolo[4,3-d]pyrimidine (10 mg, 19.21 μmol, 21% yield) as an off-white solid. MS: m/z=521.40 [M+H]⁺. ¹H NMR (400 MHz, Chloroform-d) 8.71 (s, 1H), 7.64-7.62 (m, 2H), 7.54-7.52 (m, 2H), 7.39 (s, 1H), 4.53 (s, 2H), 3.92 (s, 3H), 3.80 (s, 3H), 2.84 (s, 3H), 1.62-1.61 (m, 1H), 1.28-1.26 (m, 2H), 0.86-0.85 (m, 2H). ¹⁹F NMR (376 MHz, Chloroform-d) δ −62.15.

### Example 77. 2-(4-cyclopropyl-6-methoxy-pyrimidin-5-yl)-6-fluoro-7-[[4-[1-methyl-4-(trifluoromethyl)imidazol-2-yl]phenyl]methyl]-5H-pyrrolo[3,2-d]pyrimidine Compound 181

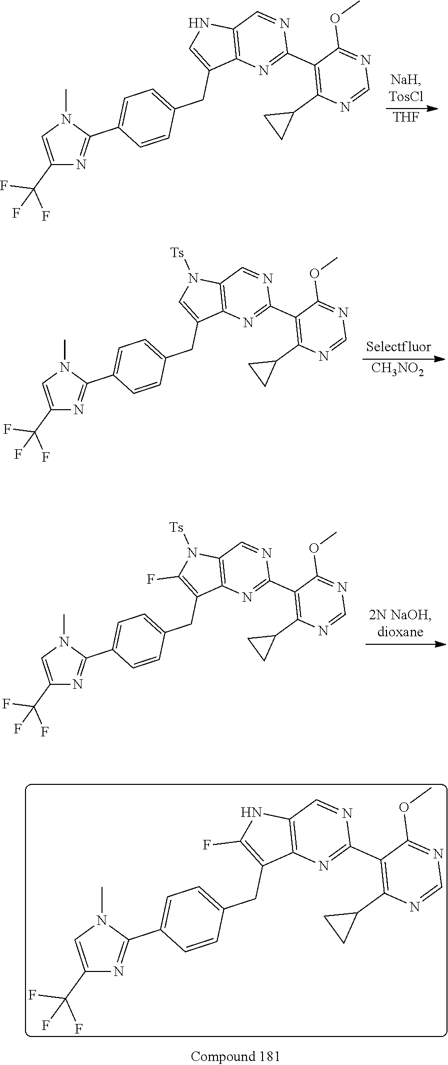

### 1.1 2-(4-cyclopropyl-6-methoxy-pyrimidin-5-yl)-7-[[4-[1-methyl-4-(trifluoromethyl)imidazol-2-yl]phenyl]methyl]-5-(p-tolylsulfonyl)pyrrolo[3,2-d]pyrimidine

To a stirred solution of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (100 mg, 197.83 μmol) in tetrahydrofuran (1.5 mL) under nitrogen atmosphere was added sodium hydride (12 mg, 297 μmol, 60% dispersion in mineral oil) at 0° C. After stirred at 0° C. for 0.5 hour, a solution of 4-methylbenzenesulfonyl chloride (45 mg, 237 μmol) in tetrahydrofuran (0.5 mL) was added dropwise with stirring at 0° C. The mixture was stirred at 25° C. for 1 hour. The reaction progress was monitored by LCMS. The reaction was quenched by the addition of saturated ammonium chloride aqueous solution (15 mL) at 0° C. The resulting mixture was extracted with ethyl acetate (3×15 mL). The combined organic layers were dried over anhydrous sodium sulfate. After filtration, the filtrate was concentrated under reduced pressure. The residue was purified by silica gel column chromatography, eluted with 1%-10% methanol in dichloromethane to give 2-(4-cyclopropyl-6-methoxy-pyrimidin-5-yl)-7-[[4-[1-methyl-4-(trifluoromethyl)imidazol-2-yl]phenyl]methyl]-5-(p-tolylsulfonyl)pyrrolo[3,2-d]pyrimidine (80 mg, 121.27 μmol, 61.30% yield) as an off-white solid. MS: m/z=660.20 [M+H]⁺. ¹H NMR (400 MHz, Chloroform-d) δ 9.48 (s, 1H), 8.71 (s, 1H), 7.93-7.82 (m, 2H), 7.65-7.55 (m, 3H), 7.44 (d, J=8.0 Hz, 2H), 7.40-7.31 (m, 3H), 4.20 (s, 2H), 3.94 (s, 3H), 3.80 (s, 3H), 2.45 (s, 3H), 1.68-1.60 (m, 1H), 1.33-1.21 (m, 2H), 0.93-0.84 (m, 2H).

### 1.2 2-(4-cyclopropyl-6-methoxy-pyrimidin-5-yl)-6-fluoro-7-[[4-[1-methyl-4-(trifluoromethyl)imidazol-2-yl]phenyl]methyl]-5-(p-tolylsulfonyl)pyrrolo[3,2-d]pyrimidine

To a mixture of 2-(4-cyclopropyl-6-methoxy-pyrimidin-5-yl)-7-[[4-[1-methyl-4-(trifluoromethyl)imidazol-2-yl]phenyl]methyl]-5-(p-tolylsulfonyl)pyrrolo[3,2-d]pyrimidine (80 mg, 121 μmol) in nitromethane (3.2 mL) under nitrogen atmosphere was added 1-(chloromethyl)-4-fluoro-1,4-diazoniabicyclo[2,2,2]octane ditetrafluoroborate (77 mg, 218 μmol) at 0° C. The resulted mixture was stirred at 25° C. for 16 hours. The reaction progress was monitored by LCMS. The resulting mixture was concentrated under reduced pressure. The residue was purified by silica gel column chromatography, eluted with 1%-50% ethyl acetate in petroleum ether to give 2-(4-cyclopropyl-6-methoxy-pyrimidin-5-yl)-6-fluoro-7-[[4-[1-methyl-4-(trifluoromethyl)imidazol-2-yl]phenyl]methyl]-5-(p-tolylsulfonyl)pyrrolo[3,2-d]pyrimidine (20 mg, 29.51 μmol, 24% yield) as a light yellow solid. MS: m/z=678.35 [M+H]⁺. ¹H NMR (400 MHz, Chloroform-d) δ 9.48 (s, 1H), 8.68 (s, 1H), 7.93-7.82 (m, 2H), 7.68-7.53 (m, 3H), 7.44 (d, J=8.0 Hz, 2H), 7.37 (d, J=8.0 Hz, 2H), 4.19 (s, 2H), 3.92 (s, 3H), 3.67 (s, 3H), 2.44 (s, 3H), 1.68-1.56 (m, 1H), 1.37-1.17 (m, 2H), 0.93-0.79 (m, 2H).

### 1.3 2-(4-cyclopropyl-6-methoxy-pyrimidin-5-yl)-6-fluoro-7-[[4-[1-methyl-4-(trifluoromethyl)imidazol-2-yl]phenyl]methyl]-5H-pyrrolo[3,2-d]pyrimidine

To a solution of 2-(4-cyclopropyl-6-methoxy-pyrimidin-5-yl)-6-fluoro-7-[[4-[1-methyl-4-(trifluoromethyl)imidazol-2-yl]phenyl]methyl]-5-(p-tolylsulfonyl)pyrrolo[3,2-d]pyrimidine (15 mg, 22 μmol) in dioxane (0.5 mL) was added sodium hydroxide (2 M, 0.5 mL) at 25° C., and then stirred at this temperature for 3 hours. The resulted mixture was diluted with water (10 mL), then extracted with ethyl acetate (3×20 mL). The combined organic layers were dried over anhydrous sodium sulfate and filtered. The filtrate was concentrated under reduced pressure. The residue was purified by Prep-HPLC with the following conditions: Column: XBridge Prep OBD C18 Column, 19×250 mm, 5 μm; Mobile Phase A: Water (10 mM aq. NH₄HCO₃), Mobile Phase B: MeOH; Flow rate: 25 mL/min; Gradient: 40% B to 45% B in 13 min, 45% B; Wave Length: 254 nm; RT1 12 min. The collected fractions were combined, concentrated and then lyophilized to give 2-(4-cyclopropyl-6-methoxy-pyrimidin-5-yl)-6-fluoro-7-[[4-[1-methyl-4-(trifluoromethyl)imidazol-2-yl]phenyl]methyl]-5H-pyrrolo[3,2-d]pyrimidine (5.1 mg, 9.74 μmol, 44% yield) as an off-white solid. MS: m/z=524.35. [M+H]⁺. ¹H NMR (400 MHz, Chloroform-d) δ 9.98 (s, 1H), 9.00 (s, 1H), 8.66 (s, 1H), 7.53 (d, J=8.0 Hz, 2H), 7.45 (d, J=8.0 Hz, 2H), 7.27 (s, 1H), 4.29 (s, 2H), 3.93 (s, 3H), 3.65 (s, 3H), 1.75-1.68 (m, 1H), 1.28-1.20 (m, 2H), 0.89-0.82 (m, 2H). ¹⁹F NMR (376 MHz, Chloroform-d) δ −61.35, −140.95.

### Example 78. 2-(4-cyclopropyl-6-methoxy-pyrimidin-5-yl)-7-[[3-fluoro-4-[1-methyl-4-(trifluoromethyl)imidazol-2-yl]phenyl]methyl]-5H-pyrrolo[3,2-d]pyrimidine Compound 153

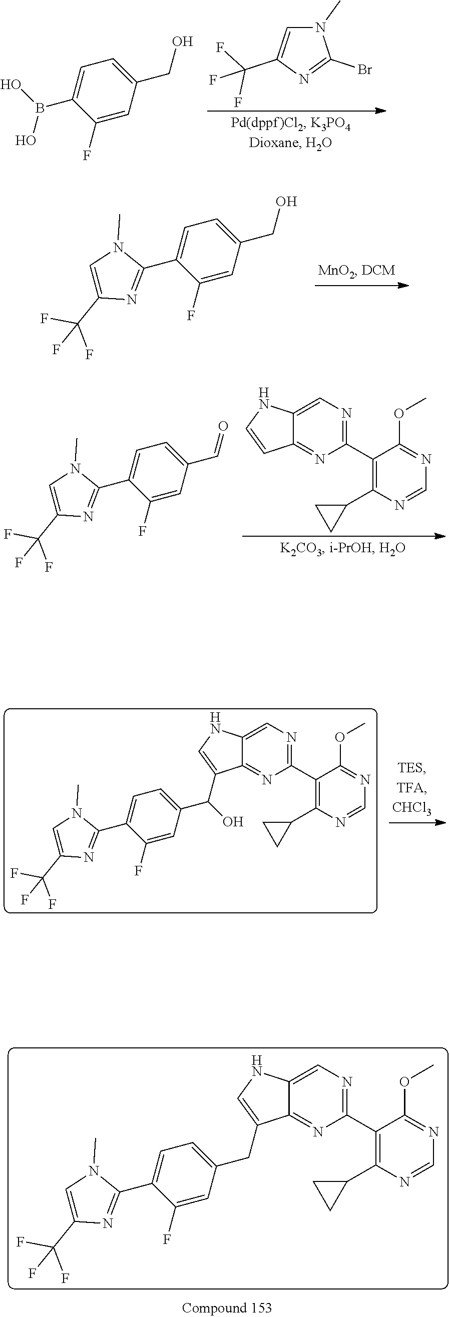

### 1.1 [3-fluoro-4-[1-methyl-4-(trifluoromethyl)imidazol-2-yl]phenyl]methanol

To a solution of [2-fluoro-4-(hydroxymethyl)phenyl]boronic acid (950 mg, 5.59 mmol) and 2-bromo-1-methyl-4-(trifluoromethyl)imidazole (1.28 g, 5.59 mmol) in 1,4-dioxane (10 mL) and water (2 mL) were added 1,1′-Bis(diphenylphosphino)ferrocene palladium(II)dichloride (456 mg, 0.559 mmol) and potassium phosphate (3.56 g, 16.77 mmol) at 25° C. under nitrogen atmosphere. The reaction mixture was heated to 90° C., and stirred for 16 hours under nitrogen atmosphere. The mixture was cooled down to 25° C. The resulted reaction was diluted with ethyl acetate (500 mL) and washed with brine (3×200 mL), dried over anhydrous sodium sulfate. After filtration, the filtrate was concentrated under reduced pressure and the residue was purified by silica gel column chromatography, eluted with 60%-70% ethyl acetate in petroleum ether to give [3-fluoro-4-[1-methyl-4-(trifluoromethyl)imidazol-2-yl]phenyl]methanol (950 mg, 3.46 mmol, 62% yield) as a yellow solid. MS: m/z=275.15 [M+H]⁺. ¹H NMR (400 MHz, Chloroform-d) δ 7.51 (t, J=7.6 Hz, 1H), 7.38 (s, 1H), 7.25-7.16 (m, 2H), 4.76 (s, 2H), 3.65 (s, 3H).

### 1.2 3-fluoro-4-[-methyl-4-(trifluoromethyl)imidazol-2-yl]benzaldehyde

To a solution of [3-fluoro-4-[1-methyl-4-(trifluoromethyl)imidazol-2-yl]phenyl]methanol (470 mg, 1.71 mmol) in dichloromethane (20 mL) was added manganese dioxide (2.98 g, 34.28 mmol) at 25° C. under nitrogen atmosphere. The resulting reaction was heated to 40° C., and stirred for 5 hours under nitrogen atmosphere. The mixture was cooled down to 25° C. After filtration, the filtrate was concentrated under reduced pressure. The residue was purified by silica gel column chromatography, eluted with 40%-45% ethyl acetate in petroleum ether to afford 3-fluoro-4-[1-methyl-4-(trifluoromethyl)imidazol-2-yl]benzaldehyde (400 mg, 1.47 mmol, 85% yield) as a light-yellow solid. MS: m/z=272.95 [M+H]⁺. ¹H NMR (400 MHz, Chloroform-d) δ 10.08 (s, 1H), 7.90-7.86 (m, 1H), 7.84-7.81 (m, 1H), 7.75-7.71 (m, 1H), 7.43 (s, 1H), 3.72 (s, 3H).

### 1.3 [2-(4-cyclopropyl-6-methoxy-pyrimidin-5-yl)-5H-pyrrolo[3,2-d]pyrimidin-7-yl]-[3-fluoro-4-[1-methyl-4-(trifluoromethyl)imidazol-2-yl]phenyl]methanol

To a solution of 2-(4-cyclopropyl-6-methoxy-pyrimidin-5-yl)-5H-pyrrolo[3,2-d]pyrimidine (170 mg, 636 μmol) and 3-fluoro-4-[1-methyl-4-(trifluoromethyl)imidazol-2-yl]benzaldehyde (173 mg, 636 μmol) in isopropanol (5 mL) and water (5 mL) was added potassium carbonate (105 mg, 763 μmol) at 25° C. under nitrogen atmosphere. The mixture was heated to 60° C., and stirred for 16 hours under nitrogen atmosphere. The mixture was cooled down to 25° C. Then the mixture was diluted with ethyl acetate (200 mL) and washed with brine (3×100 mL), dried over anhydrous sodium sulfate. After filtration, the filtrate was concentrated under reduced pressure and the residue was purified by silica gel column chromatography, eluted with 5%-7% methyl alcohol in dichloromethane to give a crude product. The crude product was further purified by RP-Flash chromatography with the following conditions: Column: C18 spherical, 20-35 um, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 42% B in 10 min, 42% B to 42% B in 4 min, 42% B to 95% B in 10 min; Detector: UV 254 & 210 nm; RT: 19 min. The fractions of first eluting peak (RT: 19 min) were collected and concentrated under reduced pressure and then lyophilized overnight to afford [2-(4-cyclopropyl-6-methoxy-pyrimidin-5-yl)-5H-pyrrolo[3,2-d]pyrimidin-7-yl]-[3-fluoro-4-[1-methyl-4-(trifluoromethyl)imidazol-2-yl]phenyl]methanol (110 mg, 203.90 μmol, 32% yield) as a white solid. MS: m/z=540.25 [M+H]⁺. ¹H NMR (400 MHz, Chloroform-d) δ 10.92 (s, 1H), 8.91 (s, 1H), 8.65 (s, 1H), 7.57-7.51 (m, 1H), 7.47-7.43 (m, 2H), 7.41-7.38 (m, 1H), 7.14 (s, 1H), 6.36 (s, 1H), 3.90 (s, 3H), 3.68 (s, 3H), 1.74-1.67 (m, 1H), 1.21-1.17 (m, 2H), 0.90-0.82 (m, 2H). ¹⁹F NMR (376 MHz, Chloroform-d) δ −62.40, −112.65.

### 1.4 2-(4-cyclopropyl-6-methoxy-pyrimidin-5-yl)-7-[[3-fluoro-4-[1-methyl-4-(trifluoromethyl)imidazol-2-yl]phenyl]methyl]-5H-pyrrolo[3,2-d]pyrimidine

To a solution of [2-(4-cyclopropyl-6-methoxy-pyrimidin-5-yl)-5H-pyrrolo[3,2-d]pyrimidin-7-yl]-[3-fluoro-4-[1-methyl-4-(trifluoromethyl)imidazol-2-yl]phenyl]methanol (100 mg, 185.36 μmol) in chloroform (5 mL) were added triethylsilane (3.64 g, 31.30 mmol, 5 mL) and trifluoroacetic acid (3.70 g, 32.45 mmol, 2.5 mL) at 0° C. under nitrogen atmosphere. Then the reaction mixture was stirred at 25° C. for 48 hours. The reaction solution was concentrated under vacuum. The residue was purified by RP-Flash chromatography with the following conditions: Column: C18 spherical Column, 20-35 um, 100A, 40 g; Mobile Phase A: Water (5 mM aq. NH₄HCO₃), Mobile Phase B: MeCN; Flow rate: 40 mL/min; Gradient: 5% B to 5% B in 5 min, 5% B to 50% B in 10 min, 50% B to 50% B in 3 min, 50% B to 95% B in 5 min; Detector: UV 254 & 210 nm; RT: 18 min. The fractions of first eluting peak (RT: 18 min) were collected and concentrated under reduced pressure and then lyophilized overnight to afford 2-(4-cyclopropyl-6-methoxy-pyrimidin-5-yl)-7-[[3-fluoro-4-[1-methyl-4-(trifluoromethyl)imidazol-2-yl]phenyl]methyl]-5H-pyrrolo[3,2-d]pyrimidine (73.8 mg, 140.98 μmol, 76.06% yield) as a white solid. MS: m/z=524.35 [M+H]⁺. ¹H NMR (400 MHz, Chloroform-d) δ 10.62 (s, 1H), 9.02 (s, 1H), 8.66 (s, 1H), 7.49 (t, J=8.0 Hz, 1H), 7.42 (s, 1H), 7.28 (s, 1H), 7.25-7.18 (m, 2H), 4.29 (s, 2H), 3.93 (s, 3H), 3.67 (s, 3H), 1.76-1.69 (m, 1H), 1.25-1.19 (m, 2H), 0.90-0.86 (m, 2H). ¹⁹F NMR (376 MHz, Chloroform-d) δ −62.39, −113.41.

### Example 79. 2-(4-cyclopropyl-6-methoxy-pyrimidin-5-yl)-7-[[2-fluoro-4-[1-methyl-4-(trifluoromethyl)imidazol-2-yl]phenyl]methyl]-5H-pyrrolo[3,2-d]pyrimidine Compound 120

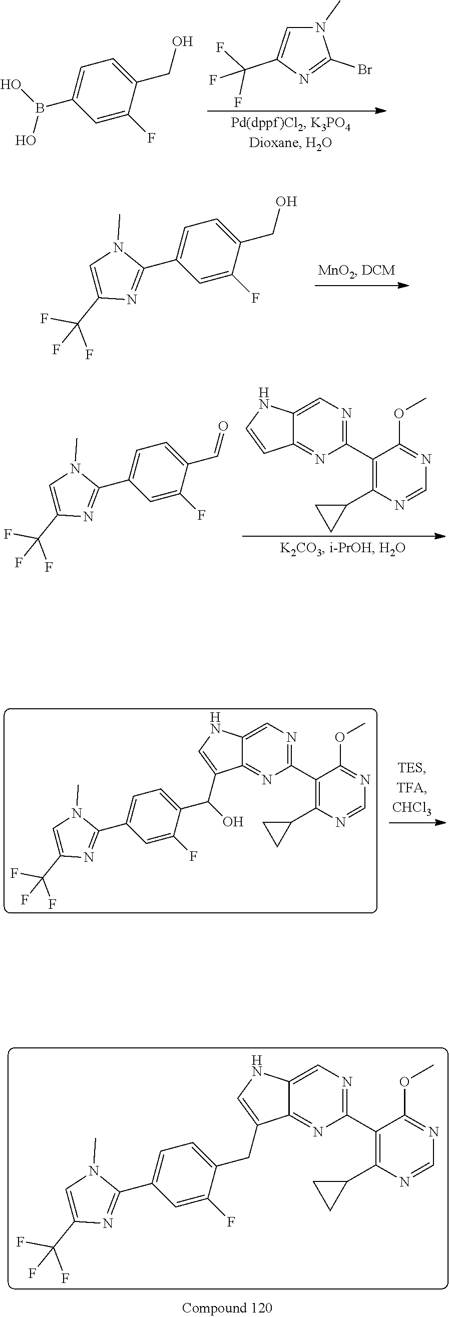

### 1.1 [2-fluoro-4-[1-methyl-4-(trifluoromethyl)imidazol-2-yl]phenyl]methanol

To a solution of [3-fluoro-4-(hydroxymethyl)phenyl]boronic acid (1 g, 5.88 mmol) and 2-bromo-1-methyl-4-(trifluoromethyl)imidazole (1.35 g, 5.88 mmol) in 1,4-dioxane (10 mL) and water (2 mL) were added [1,1′-Bis(diphenylphosphino)ferrocene]dichloropalladium(II) complex with dichloromethane (482 mg, 588 μmol) and potassium phosphate (3.75 g, 17.65 mmol). The solution was stirred for 2 hours at 100° C. under nitrogen atmosphere. The resulted mixture was concentrated under reduced pressure. The resulting mixture was diluted with water (100 mL), was extracted with ethyl acetate (3×100 mL). The combined organic layers were dried over anhydrous sodium sulfate. After filtration, the filtrate was concentrated under reduced pressure. The residue was purified by silica gel column chromatography, eluted with ethyl acetate in petroleum ether (0%-56%) to afford [2-fluoro-4-[1-methyl-4-(trifluoromethyl)imidazol-2-yl]phenyl]methanol (1.33 g, 4.85 mmol, 82% yield) as a yellow solid. MS: m/z=275.05 [M+H]⁺. ¹H NMR (400 MHz, Chloroform-d) δ 7.60-7.48 (m, 1H), 7.44-7.38 (m, 1H), 7.37-7.33 (m, 2H), 4.83 (s, 2H), 3.80 (s, 3H).

### 1.2 2-fluoro-4-[1-methyl-4-(trifluoromethyl)imidazol-2-yl]benzaldehyde

A mixture of [2-fluoro-4-[1-methyl-4-(trifluoromethyl)imidazol-2-yl]phenyl]methanol (0.5 g, 1.82 mmol) and dioxomanganese (3.17 g, 36.47 mmol) in dicholoromethane (13 mL) was stirred at 40° C. for 16 hours. The mixture was diluted by ethyl acetate, then was filtered through a Celite pad, the filtrate was concentrated under reduced pressure to afford a residue. The residue was purified by silica gel column chromatography, eluted with ethyl acetate in petroleum ether (0%-42%) to afford 2-fluoro-4-[1-methyl-4-(trifluoromethyl)imidazol-2-yl]benzaldehyde (400 mg, 1.47 mmol, 80% yield) as an off-white solid. MS: m/z=272.95 [M+H]⁺. ¹H NMR (400 MHz, Chloroform-d) δ 10.43 (s, 1H), 8.05-7.96 (m, 1H), 7.65-7.56 (m, 2H), 7.40 (s, 1H), 3.89 (s, 3H).

### 1.3 [2-(4-cyclopropyl-6-methoxy-pyrimidin-5-yl)-5H-pyrrolo[3,2-d]pyrimidin-7-yl]-[2-fluoro-4-[1-methyl-4-(trifluoromethyl)imidazol-2-yl]phenyl]methanol

To a stirred solution of 2-fluoro-4-[1-methyl-4-(trifluoromethyl)imidazol-2-yl]benzaldehyde (400 mg, 1.47 mmol) and 2-(4-cyclopropyl-6-methoxy-pyrimidin-5-yl)-5H-pyrrolo[3,2-d]pyrimidine (393 mg, 1.47 mmol) in water (2 mL) and isopropyl alcohol (2 mL) was added potassium carbonate (244 mg, 1.76 mmol) at 0° C. The mixture was stirred at 60° C. for 6 hours, 60% desired product could be detected by LCMS. The resulting mixture was diluted with water (100 mL). The resulted mixture was extracted with methanol in dicholoromethane (10%, 3×100 mL). The combined organic layers were dried over anhydrous sodium sulfate. After filtration, the filtrate was concentrated under reduced pressure. The residue was purified by reverse phase chromatography with the following conditions: Column: Spherical C18, 20-40 um, 80 g; Mobile Phase A: Water (5 mM aq. NH₄HCO₃), Mobile Phase B: ACN; Flow rate: 60 mL/min; Gradient (B %): 5% hold 5 min, 5%-38% within 8 min, 38% hold 2 min; 38%-95% within 10 min, 95% hold 5 min; Detector: UV 254 & 210 nm; RT: 13 min. The collected fractions were combined, concentrated under reduced pressure and lyophilized to afford [2-(4-cyclopropyl-6-methoxy-pyrimidin-5-yl)-5H-pyrrolo[3,2-d]pyrimidin-7-yl]-[2-fluoro-4-[1-methyl-4-(trifluoromethyl)imidazol-2-yl]phenyl]methanol (330 mg, 611.70 μmol, 41% yield) as an off-white solid. MS: m/z=540.20 [M+H]⁺. ¹H NMR (400 MHz, Chloroform-d) δ 10.51 (s, 1H), 8.96 (s, 1H), 8.66 (s, 1H), 7.84 (t, J=7.6 Hz, 1H), 7.46-7.33 (m, 3H), 7.23 (s, 1H), 6.64 (s, 1H), 4.88 (s, 1H), 3.91 (s, 3H), 3.81 (s, 3H), 1.79-1.68 (m, 1H), 1.24-1.17 (m, 2H), 0.90-0.83 (m, 21H). ¹⁹F NMR (376 MHz, Chloroform-d) δ −62.57, −116.65.

### 1.4 2-(4-cyclopropyl-6-methoxy-pyrimidin-5-yl)-7-[[2-fluoro-4-[1-methyl-4-(trifluoromethyl)imidazol-2-yl]phenyl]methyl]-5H-pyrrolo[3,2-d]pyrimidine

A mixture of [2-(4-cyclopropyl-6-methoxy-pyrimidin-5-yl)-5H-pyrrolo[3,2-d]pyrimidin-7-yl]-[2-fluoro-4-[1-methyl-4-(trifluoromethyl)imidazol-2-yl]phenyl]methanol (70 mg, 130 μmol) in chloroform (3 mL) were added trifluoroacetic acid (1.5 mL) and triethyl silicane (3 mL). The resulted solution was stirred at room temperature for 16 hours. The reaction was concentrated under reduced pressure. The residue was purified by reverse phase chromatography with the following conditions: Column: Spherical C18, 20-40 um, 40 g; Mobile Phase A: Water (5 mM aq. NH₄HCO₃), Mobile Phase B: ACN; Flow rate: 40 mL/min; Gradient (B %): 5% hold 5 min, 5%-47% within 17 min, 47% hold 2 min; 47%-95% within 10 min, 95% hold 5 min; Detector: UV 254 & 210 nm; RT: 22 min. The collected fractions were combined, concentrated under reduced pressure and lyophilized to afford 2-(4-cyclopropyl-6-methoxy-pyrimidin-5-yl)-7-[[2-fluoro-4-[1-methyl-4-(trifluoromethyl)imidazol-2-yl]phenyl]methyl]-5H-pyrrolo[3,2-d]pyrimidine (49 mg, 93.60 μmol, 72% yield) as an off-white solid. MS: m/z=524.25 [M+H]⁺. ¹H NMR (400 MHz, Chloroform-d) δ 10.13 (s, 1H), 9.00 (s, 1H), 8.66 (s, 1H), 7.46 (d, J=7.6 Hz, 1H), 7.44-7.34 (m, 3H), 7.34-7.29 (m, 1H), 4.31 (s, 2H), 3.93 (s, 3H), 3.80 (s, 3H), 1.75-1.71 (m, 1H), 1.28-1.22 (m, 2H), 0.95-0.85 (m, 2H). ¹⁹F NMR (376 MHz, Chloroform-d) δ −62.61, −116.42.

### Example 80. 2-(4-cyclopropyl-6-methoxy-pyrimidin-5-yl)-7-[[2-methyl-4-[1-methyl-4-(trifluoromethyl)imidazol-2-yl]phenyl]methyl]-5H-pyrrolo[3,2-d]pyrimidine Compound 203

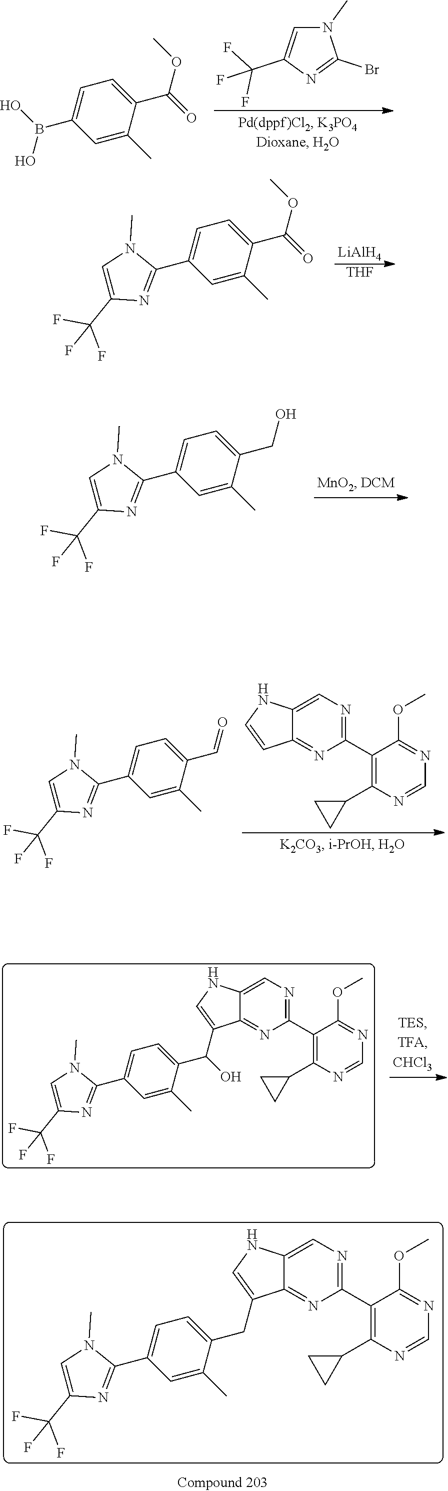

### 1.1 methyl 2-methyl-4-[1-methyl-4-(trifluoromethyl)imidazol-2-yl]benzoate

To a solution of (4-methoxycarbonyl-3-methyl-phenyl)boronic acid (1 g, 5.15 mmol) and 2-bromo-1-methyl-4-(trifluoromethyl)imidazole (1.18 g, 5.15 mmol) in water (2 mL) and 1,4-dioxane (10 mL) were added 1,1′-Bis(diphenylphosphino)ferrocene-palladium(II)dichloride (421 mg, 515 μmol) and potassium phosphate (3.28 g, 15.46 mmol) at 25° C. The mixture was degassed with nitrogen for 3 times and then stirred at 100° C. for 16 hours. The resulted mixture was cooled down to room temperature, diluted with ethyl acetate (100 mL), washed with brine (3×20 mL). The organic layer was dried over anhydrous sodium sulfate. After filtration, the filtrate was concentrated under reduced pressure. The residue was purified by silica gel column chromatography, eluted with 1%-80% ethyl acetate in petroleum ether to give methyl 2-methyl-4-[1-methyl-4-(trifluoromethyl)imidazol-2-yl]benzoate (900 mg, 3.02 mmol, 59% yield) as a yellow solid. MS: m/z=299.20 [M+H]⁺. ¹H NMR (400 MHz, Chloroform-d) δ 8.03 (d, J=8.0 Hz, 1H), 7.61 (s, 1H), 7.52 (d, J=8.0 Hz, 1H), 7.36 (s, 1H), 3.95 (s, 3H), 3.82 (s, 3H), 2.68 (s, 3H). ¹⁹F NMR (376 MHz, Chloroform-d) δ −62.76.

### 1.2 [2-methyl-4-[1-methyl-4-(trifluoromethyl)imidazol-2-yl]phenyl]methanol

To a solution of methyl 2-methyl-4-[1-methyl-4-(trifluoromethyl)imidazol-2-yl]benzoate (880 mg, 2.95 mmol) in tetrahydrofuran (10 mL) under nitrogen atmosphere was added lithium aluminum hydride (123 mg, 3.25 mmol) at 0° C., and then stirred at 25° C. for 16 hours. The reaction progress was monitored by LCMS. The resulting mixture was quenched with ethyl acetate (100 mL) and the pH value of the solution was adjusted to 3˜4 with 1 N hydrochloric acid. The resulted mixture was washed with brine (3×50 mL), dried over anhydrous sodium sulfate. After filtration, the filtrate was concentrated under reduced pressure. The residue was purified by silica gel column chromatography, eluted with 1%-80% ethyl acetate in petroleum ether to give [2-methyl-4-[1-methyl-4-(trifluoromethyl)imidazol-2-yl]phenyl]methanol (750 mg, 2.78 mmol, 94% yield) as a yellow solid. MS: m/z=271.20 [M+H]⁺. ¹H NMR (400 MHz, Chloroform-d) δ 7.46 (d, J=8.0 Hz, 2H), 7.38 (d, J=8.0 Hz, 1H), 7.32 (s, 1H), 4.75 (s, 2H), 3.76 (s, 3H), 2.37 (s, 3H). ¹⁹F NMR (376 MHz, Chloroform-d) δ −62.64.

### 1.3 2-methyl-4-[1-methyl-4-(trifluoromethyl)imidazol-2-yl]benzaldehyde

To a solution of [2-methyl-4-[1-methyl-4-(trifluoromethyl)imidazol-2-yl]phenyl]methanol (200 mg, 740 μmol) in dichloromethane (4 mL) was added dioxomanganese (1.29 g, 14.80 mmol) at 25° C. The resulted solution was stirred at 40° C. for 16 hours. The resulting mixture was cooled down to room temperature and the solid was filtered out. The filtrate was concentrated under reduced pressure. The residue was purified by silica gel column chromatography, eluted with 1%-60% ethyl acetate in petroleum ether to give 2-methyl-4-[1-methyl-4-(trifluoromethyl)imidazol-2-yl]benzaldehyde (180 mg, 671.06 μmol, 91% yield) as a yellow solid. MS: m/z=269.00 [M+H]⁺. ¹H NMR (400 MHz, Chloroform-d) δ 10.35 (s, 1H), 7.92 (d, J=8.0 Hz, 1H), 7.63 (d, J=8.8 Hz, 2H), 7.38 (s, 1H), 3.85 (s, 3H), 2.75 (s, 3H). ¹⁹F NMR (376 MHz, Chloroform-d) δ −62.75.

### 1.4 [2-(4-cyclopropyl-6-methoxy-pyrimidin-5-yl)-5H-pyrrolo[3,2-d]pyrimidin-7-yl]-[2-methyl-4-[1-methyl-4-(trifluoromethyl)imidazol-2-yl]phenyl]methanol

To a solution of 2-methy-4-[1-methyl-4-(trifluoromethyl)imidazol-2-yl]benzaldehyde (160 mg, 596 μmol) and 2-(4-cyclopropyl-6-methoxy-pyrimidin-5-yl)-5H-pyrrolo[3,2-d]pyrimidine (159 mg, 596 μmol) in water (2 mL) and propan-2-ol (2 mL) was added potassium carbonate (99 mg, 716 μmol) at 25° C., and then stirred at 60° C. for 16 hours. The resulted mixture was cooled down to mom temperature and then diluted with ethyl acetate (50 mL), washed with brine (3×20 mL). The organic layer was dried over anhydrous sodium sulfate. After filtration, the filtrate was concentrated under reduced pressure. The residue was purified by RP-Flash with the following conditions: Column: Spherical C18, 20-40 um, 40 g; Mobile Phase A: Water (5 mM aq. NH₄HCO₃), Mobile Phase B: MeCN; Flow rate: 40 mL/min; Gradient (B %): 2% B to 2% B in 5 min, 2% B to 46% B in 15 min, 46% B to 46% B in 3 min, 46% B to 95% B in 10 min, Detector: UV 254 & 210 nm; RT: 21 min. The collected fractions were combined and concentrated under reduced pressure to give [2-(4-cyclopropyl-6-methoxy-pyrimidin-5-yl)-5H-pyrrolo[3,2-d]pyrimidin-7-yl]-[2-methyl-4-[1-methyl-4-(trifluoromethyl)imidazol-2-yl]phenyl]methanol (40 mg, 74.69 μmol, 13% yield) as an off-white solid. MS: m/z=536.25 [M+H]⁺. ¹H NMR (400 MHz, Methanol-d₄) δ 9.00 (s, 1H), 8.65 (s, 1H), 7.87 (d, J=8.0 Hz, 1H), 7.70 (s, 1H), 7.60 (s, 1H), 7.56-7.54 (m, 1H), 7.46 (s, 1H), 6.60 (s, 1H), 3.93 (s, 3H), 3.80 (s, 3H), 2.36 (s, 3H), 1.68-1.62 (m, 1H), 1.19-1.12 (m, 2H), 0.96-0.85 (m, 2H). ¹⁹F NMR (376 MHz, Methanol-d₄) δ −63.91.

### 1.5 2-(4-cyclopropyl-6-methoxy-pyrimidin-5-yl)-7-[[2-methyl-4-[1-methyl-4-(trifluoromethyl)imidazol-2-yl]phenyl]methyl]-5H-pyrrolo[3,2-d]pyrimidine

To a stirred solution of [2-(4-cyclopropyl-6-methoxy-pyrimidin-5-yl)-5H-pyrrolo[3,2-d]pyrimidin-7-yl]-[2-methyl-4-[1-methyl-4-(trifluoromethyl)imidazol-2-yl]phenyl]methanol (35 mg, 65 μmol) in triethylsilane (0.4 mL) and chloroform (0.4 mL) was added trifluoroacetic acid (0.2 mL) at 25° C., and then stirred at this temperature for 2 hours. The resulted mixture was concentrated under reduced pressure. The residue was purified by RP-Flash with the following conditions: Column: Spherical C18, 20-40 um, 40 g; Mobile Phase A: Water (5 mM aq. NH₄HCO₃), Mobile Phase B: MeCN; Flow rate: 40 mL/min; Gradient (B %): 2% B to 2% B in 5 min, 2% B to 46% B in 15 min, 46% B to 46% B in 3 min, 46% B to 95% B in 10 min, Detector: UV 254 & 210 nm; RT: 21 min. The collected fractions were combined and concentrated under reduced pressure and then lyophilized to give 2-(4-cyclopropyl-6-methoxy-pyrimidin-5-yl)-7-[[2-methyl-4-[1-methyl-4-(trifluoromethyl)imidazol-2-yl]phenyl]methyl]-5H-pyrrolo[3,2-d]pyrimidine (25.2 mg, 48.51 μmol, 74% yield) as an off-white solid. MS: m/z=520.25 [M+H]⁺. ¹H NMR (400 MHz, Chloroform-d) δ 10.01 (s, 1H), 9.02 (s, 1H), 8.67 (s, 1H), 7.48 (s, 1H), 7.37-7.35 (m, 2H), 7.32-7.28 (m, 1H), 7.10 (s, 1H), 4.25 (s, 2H), 3.94 (s, 3H), 3.80 (s, 3H), 2.37 (s, 3H), 1.77-1.71 (m, 1H) 1.28-1.22 (m, 2H), 0.92-0.85 (m, 2H). ¹⁹F NMR (376 MHz, Chloroform-d) δ −62.48.

### Example 81. 5-(4-cyclopropyl-6-methoxy-pyrimidin-5-yl)-3-[[2-fluoro-4-[1-methyl-4-(trifluoromethyl)imidazol-2-yl]phenyl]methyl]-1H-pyrazolo[4,3-d]pyrimidine Compound 189

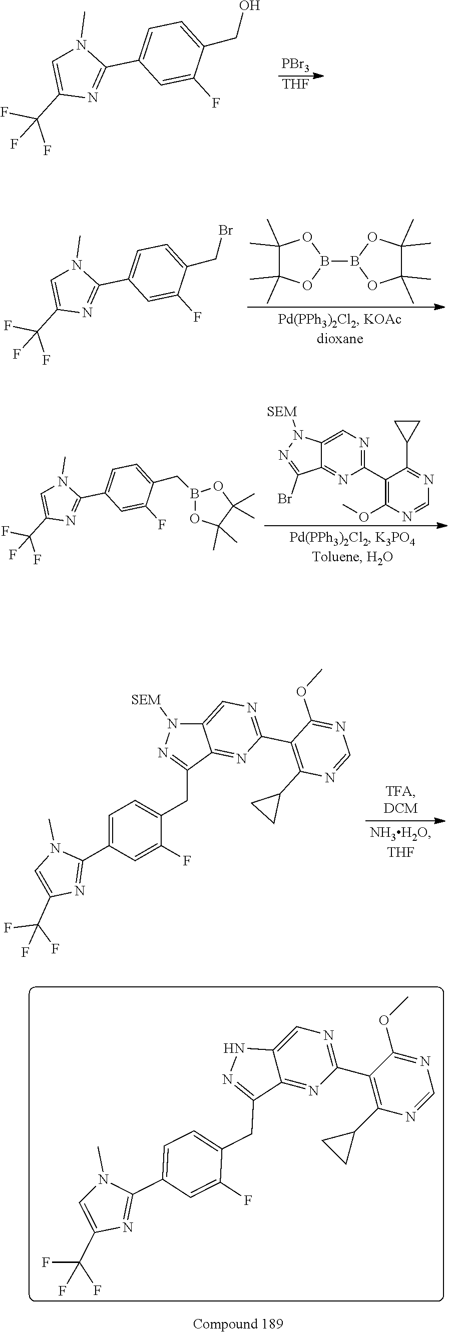

### 1.1 2-[4-(bromomethyl)-3-fluoro-phenyl]-1-methyl-4-(trifluoromethyl)imidazole

To a stirred solution of [2-fluoro-4-[1-methyl-4-(trifluoromethyl)imidazol-2-yl]phenyl]methanol (400 mg, 1.46 mmol) in tetrahydrofuran (4 mL) was added tribromophosphane (1.18 g, 4.38 mmol) dropwise at 0° C. The mixture was stirred at room temperature for 2 hours. The reaction was quenched with saturated sodium bicarbonate aqueous solution (20 mL) and diluted with water (100 mL). The resulting mixture was extracted with ethyl acetate (3×100 mL). The combined organic layers were dried over anhydrous sodium sulfate. After filtration, the filtrate was concentrated under reduced pressure. The residue was purified by silica gel column chromatography, eluted with ethyl acetate in petroleum ether (0%-30%) to afford 2-[4-(bromomethyl)-3-fluoro-phenyl]-1-methyl-4-(trifluoromethyl)imidazole (410 mg, 1.22 mmol, 83% yield) as a yellow solid. MS: m/z=335.00, 337.00 [M+H]⁺. ¹H NMR (400 MHz, Chloroform-d) δ 7.54 (t, J=7.2 Hz, 1H), 7.49-7.39 (m, 2H), 7.35 (s, 1H), 4.57 (s, 2H), 3.84 (s, 3H).

### 1.2 2-[3-fluoro-4-[(4,4,5,5-tetramethyl-1,3,2-dioxaborolan-2-yl)methyl]phenyl]-1-methyl-4-(trifluoromethyl)imidazole

To a stirred solution of 2-[4-(bromomethyl)-3-fluoro-phenyl]-1-methyl-4-(trifluoromethyl)imidazole (340 mg, 1.01 mmol) and 4,4,5,5-tetramethyl-2-(4,4,5,5-tetramethyl-1,3,2-dioxaborolan-2-yl)-1,3,2-dioxaborolane (435 mg, 1.71 mmol) in 1,4-dioxane (4 mL) were added bis(triphenylphosphine)palladium(II) chloride (71 mg, 101 μmol) and potassium acetate (297 mg, 3.03 mmol). The mixture was stirred at 80° C. for 16 hours under nitrogen atmosphere. The reaction mixture was cooled down to room temperature. The resulted mixture was concentrated under reduced pressure. The residue was purified by silica gel column chromatography, eluted with ethyl acetate in petroleum ether (0%-28%) to afford 2-[3-fluoro-4-[(4,4,5,5-tetramethyl-1,3,2-dioxaborolan-2-yl)methyl]phenyl]-1-methyl-4-(trifluoromethyl)imidazole (190 mg, 494 μmol, 49% yield) as an off-white solid. MS: m/z=385.30 [M+H]⁺. ¹H NMR (400 MHz, Chloroform-d) δ 7.36-7.29 (m, 4H), 3.81 (s, 3H), 2.33 (d, J=2.0 Hz, 2H), 1.27 (s, 12H).

### 1.3 2-[[5-(4-cyclopropyl-6-methoxy-pyrimidin-5-yl)-3-[[2-fluoro-4-[1-methyl-4-(trifluoromethyl)imidazol-2-yl]phenyl]methyl]pyrazolo[4,3-d]yrimidin-1-yl]methoxy]ethyl-trimethyl-silane

To a solution of 2-[3-fluoro-4-[(4,4,5,5-tetramethyl-1,3,2-dioxaborolan-2-yl)methyl]phenyl]-1-methyl-4-(trifluoromethyl)imidazole (170 mg, 442.51 μmol) and 2-[[3-bromo-5-(4-cyclopropyl-6-methoxy-pyrimidin-5-yl)pyrazolo[4,3-d]pyrimidin-1-yl]methoxy]ethyl-trimethyl-silane (211 mg, 442 μmol) in toluene (2 mL) and water (0.4 mL) were added potassium phosphate (188 mg, 885 μmol) and bis(triphenylphosphine)palladium(II) chloride (93 mg, 133 μmol). The solution was stirred at 90° C. for 16 hours under nitrogen atmosphere, 45% desired product could be detected by LCMS. The reaction mixture was cooled down to room temperature and diluted with water (100 mL). The resulted mixture was extracted with ethyl acetate (3×100 mL). The combined organic layers were dried over anhydrous sodium sulfate. After filtration, the filtrate was concentrated under reduced pressure. The residue was purified by silica gel column chromatography, eluted with ethyl acetate in petroleum ether (0%-100%) to afford 2-[[5-(4-cyclopropyl-6-methoxy-pyrimidin-5-yl)-3-[[2-fluoro-4-[1-methyl-4-(trifluoromethyl)imidazol-2-yl]phenyl]methyl]pyrazolo[4,3-d]pyrimidin-1-yl]methoxy]ethyl-trimethyl-silane (120 mg, 183.28 μmol, 41% yield) as a light yellow oil. MS: m/z=655.45

### 1.4 5-(4-cyclopropyl-6-methoxy-pyrimidin-5-yl)-3-[[2-fluoro-4-[1-methyl-4-(trifluoromethyl)imidazol-2-yl]phenyl]methyl]-1H-pyrazolo[4,3-d]pyrimidine

A mixture of 2-[[5-(4-cyclopropyl-6-methoxy-pyrimidin-5-yl)-3-[[2-fluoro-4-[1-methyl-4-(trifluoromethyl)imidazol-2-yl]phenyl]methyl]pyrazolo[4,3-d]pyrimidin-1-yl]methoxy]ethyl-trimethyl-silane (60 mg, 91.64 μmol) in dichloromethane (2 mL) and trifluoroacetic acid (2 mL) was stirred at room temperature for 2 hours. The resulting mixture was concentrated under reduced pressure. To a stirred solution of the residue in tetrahydrofuran (2 mL) was added ammonium hydroxide (28% aqueous solution, 2 mL) at 25° C. under nitrogen atmosphere and then stirred at this temperature 1 hour. The resulted mixture was concentrated under reduced pressure. The obtained product was purified by Prep-TLC (dichloromethane:methanol=10:1) to afford crude product as a light yellow oil. The crude product was purified by reverse phase chromatography with the following conditions: Column: Spherical C18, 20-40 um, 40 g; Mobile Phase A: Water (5 mM aq. NH₄HCO₃), Mobile Phase B: ACN; Flow rate: 40 mL/min; Gradient (B %): 5% hold 5 min, 5%-48% within 30 min, 48% hold 2 min; 48%-95% within 10 min, 95% hold 5 min; Detector: UV 254 & 210 nm; RT: 23 min). The collected fractions were combined and concentrated under reduced pressure to afford 5-(4-cyclopropyl-6-methoxy-pyrimidin-5-yl)-3-[[2-fluoro-4-[1-methyl-4-(trifluoromethyl)imidazol-2-yl]phenyl]methyl]-1H-pyrazolo[4,3-d]pyrimidine (23.7 mg, 45.19 μmol, 49% yield) as an off-white solid. MS: m/z=525.10 [M+H]⁺. H NMR (400 MHz, Chloroform-d) δ 12.61 (br., 1H), 9.25 (s, 1H), 8.75 (s, 1H), 7.33-7.55 (m, 4H), 4.58 (s, 2H), 3.96 (s, 3H), 3.84 (s, 3H), 1.73-1.67 (m, 1H), 1.34-1.27 (m, 1H), 0.94-0.91 (m, 2H). ¹⁹F NMR (376 MHz, Chloroform-d) δ −62.39, 115.30.

### Example 82. 5-(4-cyclopropyl-6-methoxypyrimidin-5-yl)-3-(3-fluoro-4-(1-methyl-4-(trifluoromethyl)-1H-imidazol-2-yl)benzyl)-1H-pyrazolo[4,3-d]pyrimidine Compound 90

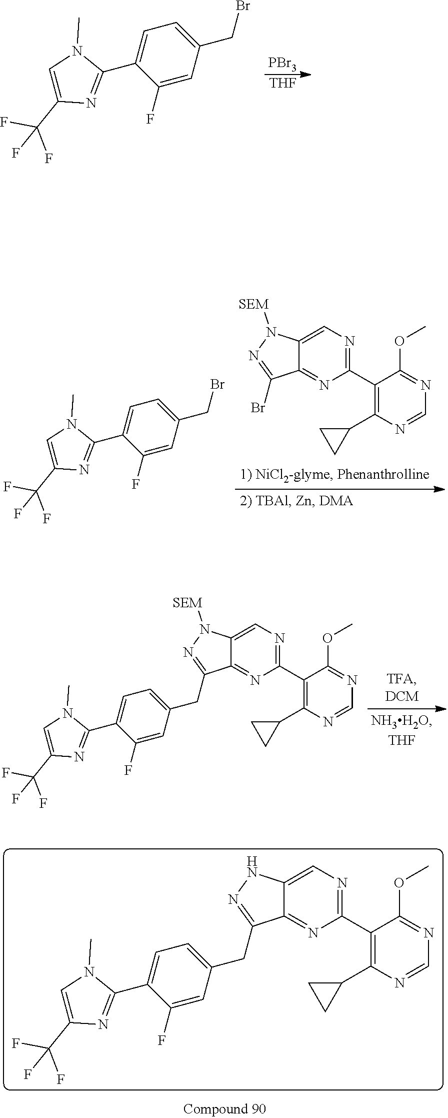

### 1.1 2-(4-(bromomethyl)-2-fluorophenyl)-1-methyl-4-(trifluoromethyl)-1H-imidazole

To a solution of [3-fluoro-4-[1-methyl-4-(trifluoromethyl)imidazol-2-yl]phenyl]methanol (400 mg, 1.46 mmol) in tetrahydrofuran (8 mL) was added tribromophosphane (1.18 g, 4.38 mmol, 411 μL) at 0° C. The reaction mixture was stirred at 25° C. for 6 hours. Product can be found by LCMS. The mixture quenched with saturated sodium bicarbonate aqueous solution (200 mL), then extracted with ethyl acetate (3×200 mL). The combined organic layers were dried over anhydrous sodium sulfate and filtered. The filtrate was concentrated under reduced pressure. The residue was purified by silica gel column chromatography, eluted with 0%-50% petroleum ether in ethyl acetate to afford 2-(4-(bromomethyl)-2-fluorophenyl)-1-methyl-4-(trifluoromethyl)-1H-imidazole (300 mg, 889.92 μmol, 61% yield) as a yellow solid. MS: m/z=337.00, 338.90 [M+H]⁺. ¹H NMR (400 MHz, Chloroform-d) δ 7.68-7.63 (m, 1H), 7.40 (s, 1H), 7.34 (d, J=7.6 Hz, 1H), 7.26 (s, 1H), 4.52 (s, 2H), 3.70 (s, 3H).

### 1.1 5-(4-cyclopropyl-6-methoxypyrimidin-5-yl)-3-(3-fluoro-4-(1-methyl-4-(trifluoromethyl)-1H-imidazol-2-yl)benzyl)-1-((2-(trimethylsilyl)ethoxy)methyl)-1H-pyrazolo[4,3-d]pyrimidine

A mixture of nickel(II) chloride dimethoxyethane adduct (21 mg, 95 μmol) and 1,10-phenanthroline (17 mg, 95 μmol) in N,N-dimethylacetamide (5 mL) was stirred for 10 min at 25° C. under nitrogen atmosphere. Then were added a mixture solution of tetrabutylammonium iodide (351 mg, 949 μmol), 2-(4-(bromomethyl)-2-fluorophenyl)-1-methyl-4-(trifluoromethyl)-1H-imidazole (320 mg, 949 μmol) and 3-bromo-5-(4-cyclopropyl-6-methoxypyrimidin-5-yl)-1-((2-(trimethylsilyl)ethoxy)methyl)-1H-pyrazolo[4,3-d]pyrimidine (453 mg, 949 μmol) in N,N-dimethylacetamide (5 mL) at 25° C. under nitrogen atmosphere. Then zinc (124 mg, 1.90 mmol) was added to the reaction mixture. The reaction mixture was stirred at 50° C. for 16 hours. Product could be found by LCMS. The filtrate was concentrated under reduced pressure. The residue was purified by Prep-TLC (petroleum ether: ethyl acetate=1:1) to afford 5-(4-cyclopropyl-6-methoxypyrimidin-5-yl)-3-(3-fluoro-4-(1-methyl-4-(trifluoromethyl)-1H-imidazol-2-yl)benzyl)-1-((2-(trimethylsilyl)ethoxy)methyl)-1H-pyrazolo[4,3-d]pyrimidine (26 mg, 39.71 μmol, 17% yield) as an off-white solid. MS: m/z=655.20 [M+H]⁺.

### 1.3 5-(4-cyclopropyl-6-methoxypyrimidin-5-yl)-3-(3-fluoro-4-(1-methyl-4-(trifluoromethyl)-1H-imidazol-2-yl)benzyl)-1H-pyrazolo[4,3-d]pyrimidine

To a solution of 5-(4-cyclopropyl-6-methoxypyrimidin-5-yl)-3-(3-fluoro-4-(1-methyl-4-(trifluoromethyl)-1H-imidazol-2-yl)benzyl)-1-((2-(trimethylsilyl)ethoxy)methyl)-1H-pyrazolo[4,3-d]pyrimidine (26 mg, 40 μmol) in dichloromethane (I mL) was added trifluoroacetic acid (1 mL) at 0° C. under nitrogen atmosphere. The reaction mixture was stirred at 25° C. for 2 hours and then concentrated under reduced pressure. The residue was dissolved in tetrahydrofuran (1 mL) and ammonium hydroxide (1 mL, 28% aqueous solution). The reaction mixture was stirred at 25° C. for 1 hour and then concentrated under reduced pressure. The residue was purified by Prep-TLC to give a crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN: Flow rate: 35 mL/min; Gradient: 5% B to 5% B in 5 min, 5% B to 65% B in 10 min, 65% B to 65% B in 3 min, 65% B to 95% B in 15 min; Detector: UV 254 & 210 nm; RT: 16 min. The collected fractions were combined, concentrated and then lyophilized overnight to afford 5-(4-cyclopropyl-6-methoxypyrimidin-5-yl)-3-(3-fluoro-4-(1-methyl-4-(trifluoromethyl)-1H-imidazol-2-yl)benzyl)-1H-pyrazolo[4,3-d]pyrimidine (8 mg, 15.25 μmol, 38% yield) as an off-white solid. MS: m/z=525.20 [M+H]⁺. ¹H NMR (400 MHz, Chloroform-d) δ 13.21 (s, 1H), 9.20 (s, 1H), 8.69 (s, 1H), 7.74 (t, J=7.6 Hz, 1H), 7.45 (s, 1H), 7.43 (s, 1H), 7.32 (d, J=8.0 Hz, 1H), 4.53 (s, 2H), 3.92 (s, 3H), 3.71 (s, 3H), 1.71-1.63 (m, 1H), 1.30-1.19 (m, 2H), 0.90-0.86 (m, 2H). ¹⁹F NMR (376 MHz, Chloroform-d) δ −62.20, −113.21.

### Example 83. 2-(4-cyclopropyl-6-methoxy-pyrimidin-5-yl)-7-[[4-[4-(1,1-difluoroethyl)-1-methyl-imidazol-2-yl]-3-methyl-phenyl]methyl]-5H-pyrrolo[3,2-d]pyrimidine Compound 113

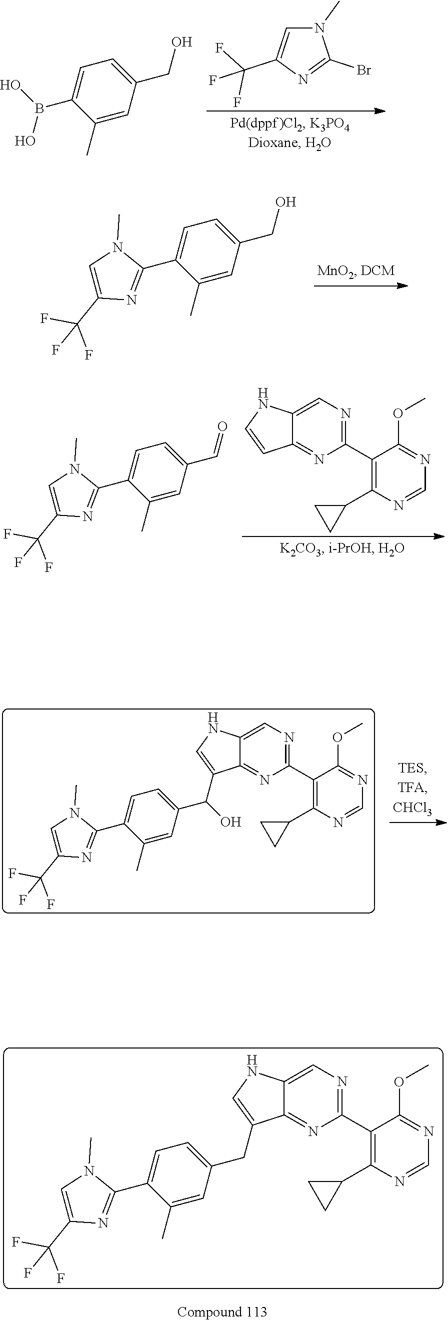

### 1.1 [3-methyl-4-[1-methyl-4-(trifluoromethyl)imidazol-2-yl]phenyl]methanol

To a stirred solution of [4-(hydroxymethyl)-2-methyl-phenyl]boronic acid (600 mg, 3.61 mmol) and 2-bromo-1-methyl-4-(trifluoromethyl)imidazole (828 mg, 3.61 mmol) in 1,4-dioxane (5 mL) and water (1 mL) were added 1,1F-bis(diphenylphosphino)ferrocene]dichloropalladium(II) dichloromethane complex (254 mg, 361 μmol) and potassium phosphate (2.30 g, 10.84 mmol) at 25° C. under nitrogen atmosphere. The resulting mixture was stirred for 16 hours at 90° C. under nitrogen atmosphere. The reaction was monitored by LCMS. The resulting mixture was concentrated under reduced pressure. The resulted mixture was diluted with ethyl acetate (10 mL). The mixture solution were filtered, the filter cake was washed with ethyl acetate (3×2 mL). The filtrate was concentrated under reduced pressure. The residue was purified by silica gel column chromatography, eluted with 1%-50% ethyl acetate in petroleum ether to give [3-methyl-4-[1-methyl-4-(trifluoromethyl)imidazol-2-yl]phenyl]methanol (800 mg, 2.96 mmol, 81% yield) as a light yellow solid. MS: m/z=271.05 [M+H]⁺. ¹H NMR (400 MHz, Chloroform-d) δ 7.39-7.34 (m, 1H), 7.32 (s, 1H), 7.30-7.24 (m, 2H), 4.74 (s, 2H), 3.54 (s, 3H), 2.24 (s, 3H).

### 1.2 3-methyl-4-[1-methyl-4-(trifluoromethyl)imidazol-2-yl]benzaldehyde

To a stirred solution of [3-methyl-4-[1-methyl-4-(trifluoromethyl)imidazol-2-yl]phenyl]methanol (800 mg, 2.96 mmol) in dichloromethane (20 mL) was added dioxomanganese (5.14 g, 59.20 mmol) at 25° C. under nitrogen atmosphere. The resulted mixture was stirred for 16 hours at 25° C. under nitrogen atmosphere. The resulted mixture were filtered, the filter cake was washed with dichloromethane (3×3 mL). The filtrate was concentrated under reduced pressure to give 3-methyl-4-[1-methyl-4-(trifluoromethyl)imidazol-2-yl]benzaldehyde (616 mg, 2.30 mmol, 77% yield) as a white solid. MS: m/z=269.00 [M+H]⁺. ¹H NMR (400 MHz, Chloroform-d) δ 10.08 (s, 1H), 7.88-7.78 (m, 2H), 7.51 (d, J=7.6 Hz, 1H), 7.39 (s, 1H), 3.58 (s, 3H), 2.36 (s, 3H).

### 1.3 [2-(4-cyclopropyl-6-methoxy-pyrimidin-5-yl)-5H-pyrrolo[3,2-d]pyrimidin-7-yl]-[3-methyl-4-[1-methyl-4-(trifluoromethyl)imidazol-2-yl]phenyl]methanol

To a stirred solution of 3-methyl-4-[1-methyl-4-(trifluoromethyl)imidazol-2-yl]benzaldehyde (200 mg, 746 μmol) and 2-(4-cyclopropyl-6-methoxy-pyrimidin-5-yl)-5H-pyrrolo[3,2-d]pyrimidine (199 mg, 746 μmol) in water (1 mL) and isopropyl alcohol (1 mL) was added potassium carbonate (124 mg, 895 μmol) at 25° C. under nitrogen atmosphere. The resulted mixture was diluted with water (10 mL). The resulted mixture was extracted with ethyl acetate (3×15 mL). The combined organic layers were washed with brine (10 mL), dried over anhydrous sodium sulfate. After filtration, the filtrate was concentrated under reduced pressure. And then purified by Prep-TLC, eluted with 10% methanol in dichloromethane to give a crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 35 mL/min; Gradient: 5% B to 5% B in 5 min, 5% B to 53% B in 11 min, 53% B to 53% B in 4 min, 53% B to 95% B in 10 min; Detector: UV 254 & 210 nm; RT: 16 min. The collected fractions were combined, concentrated and then lyophilized to give [2-(4-cyclopropyl-6-methoxy-pyrimidin-5-yl)-5H-pyrrolo[3,2-d]pyrimidin-7-yl]-[3-methyl-4-[1-methyl-4-(trifluoromethyl)imidazol-2-yl]phenyl]methanol (94.8 mg, 177.02 μmol, 23% yield) as a white solid. MS: m/z=536.25 [M+H]⁺. ¹H NMR (400 MHz, Chloroform-d) δ 10.88 (s, 1H), 8.88 (s, 1H), 8.65 (s, 1H), 7.48 (s, 1H), 7.46-7.38 (m, 2H), 7.32 (d, J=7.8 Hz, 1H), 6.99 (s, 1H), 6.35 (s, 1H), 4.57 (s, 1H), 3.92 (s, 3H), 3.56 (s, 3H), 2.20 (s, 3H), 1.76-1.70 (m, 1H), 1.22-1.20 (m, 2H), 0.92-0.81 (m, 2H).

### 1.4 2-(4-cyclopropyl-6-methoxy-pyrimidin-5-yl)-7-[[4-[4-(1,1-difluoroethyl)-1-methyl-imidazol-2-yl]-3-methyl-phenyl]methyl]-5H-pyrrolo[3,2-d]pyrimidine

To a stirred solution of [2-(4-cyclopropyl-6-methoxy-pyrimidin-5-yl)-5H-pyrrolo[3,2-d]pyrimidin-7-yl]-[3-methyl-4-[1-methyl-4-(trifluoromethyl)imidazol-2-yl]phenyl]methanol (92 mg, 171.8 μmol) in chloroform (2 mL) were added triethylsilane (2 mL) and trifluoroacetic acid (1.48 g, 12.98 mmol, 1 mL) at 25° C. under nitrogen atmosphere. The resulting mixture was stirred for 2 hours at 25° C. under nitrogen atmosphere. The reaction was monitored by LCMS. The reaction mixture was concentrated under reduced pressure and then purified by RP-Flash with the following conditions: Column: C18 spherical Column, 20-35 um, 100A, 40 g; Mobile Phase A: Water (5 mM aq. NH₄HCO₃), Mobile Phase B: MeCN: Flow rate: 35 mL/min; Gradient: 5% B to 5% B in 5 min, 5% B to 57% B in 11 min, 57% B to 57% B in 4 min, 57% B to 95% B in 10 min; Detector: UV 254 & 210 nm; RT: 16 min. The collected fractions were combined, concentrated and then lyophilized to give 2-(4-cyclopropyl-6-methoxy-pyrimidin-5-yl)-7-[[4-[4-(1,1-difluoroethyl)-1-methyl-imidazol-2-yl]-3-methyl-phenyl]methyl]-5H-pyrrolo[3,2-d]pyrimidine (52.8 mg, 102.41 μmol, 59% yield) as a white solid. MS: m/z=520.15 [M+H]⁺. ¹H NMR (400 MHz, Chloroform-d) δ 9.08 (s, 1H), 8.67 (s, 1H), 7.38 (s, 1H), 7.26-7.20 (m, 3H), 7.12 (s, 1H), 4.26 (s, 2H), 3.95 (s, 3H), 3.55 (s, 3H), 2.17 (s, 3H), 1.77-1.71 (m, 1H), 1.28-1.19 (m, 2H), 0.89-084 (m, 2H). ¹⁹F NMR (376 MHz, Chloroform-d) δ −62.48.

### Example 84. 5-(4-cyclopropyl-6-methoxy-pyrimidin-5-yl)-3-[[3-methyl-4-[1-methyl-4-(trifluoromethyl)imidazol-2-yl]phenyl]methyl]-1H-pyrazolo[4,3-d]pyrimidine Compound 95

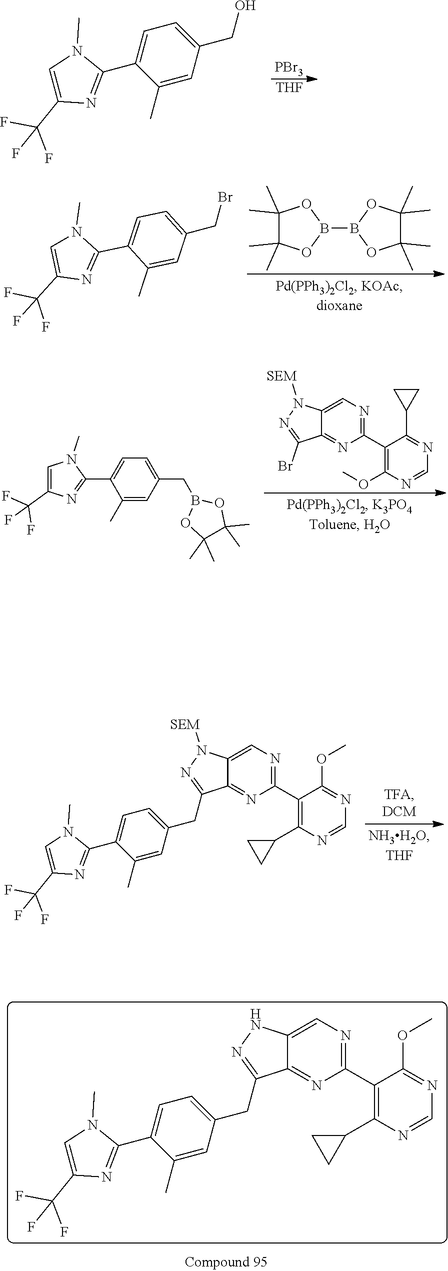

### 1.1 2-[4-(bromomethyl)-2-methyl-phenyl]-1-methyl-4-(trifluoromethyl)imidazole

To a stirred solution of [3-methyl-4-[1-methyl-4-(trifluoromethyl)imidazol-2-yl]phenyl]methanol (300 mg, 1.11 mmol) in tetrahydrofuran (3 mL) was added tribromophosphane (901 mg, 3.33 mmol, 313 μL) dropwise at 0° C. under nitrogen atmosphere. The resulted mixture was stirred for 2 hours at 25° C. under nitrogen atmosphere. The reaction was quenched by the addition of saturated sodium bicarbonate aqueous solution (10 mL) at 0° C. The resulting mixture was extracted with ethyl acetate (3×20 mL). The combined organic layers were washed with brine (10 mL), dried over anhydrous sodium sulfate. After filtration, the filtrate was concentrated under reduced pressure. The residue was purified by silica gel column chromatography, eluted with 1%-40% ethyl acetate in petroleum ether to give 2-[4-(bromomethyl)-2-methyl-phenyl]-1-methyl-4-(trifluoromethyl)imidazole (330 mg, 990.56 μmol, 89% yield) as a yellow solid. MS: m/z=332.90. 334.90 [M+H]⁺. ¹H NMR (400 MHz, Chloroform-d) δ 7.39-7.34 (m, 1H), 7.32 (s, 1H), 7.28-7.24 (m, 2H), 4.74 (s, 2H), 3.54 (s, 3H), 2.24 (s, 3H).

### 1.2 1-methyl-2-[2-methyl-4-[(4,4,5,5-tetramethyl-1,3,2-dioxaborolan-2-yl)methyl]phenyl]-4-(trifluoromethyl)imidazole

To a stirred solution of 2-[4-(bromomethyl)-2-methyl-phenyl]-1-methyl-4-(trifluoromethyl)imidazole (330 mg, 990.5 mmol) and 4,4,5,5-tetramethyl-2-(4,4,5,5-tetramethyl-1,3,2-dioxaborolan-2-yl)-1,3,2-dioxaborolane (377 mg, 1.49 mmol) in 1,4-dioxane (3 mL) were added potassium acetate (194 mg, 1.98 mmol) and bis(triphenylphosphine)palladium(II) chloride (70 mg, 99 μmol) at 25° C. under nitrogen atmosphere. The resulting mixture was stirred for 16 hours at 80° C. under nitrogen atmosphere. The resulting mixture were filtered, the filter cake was washed with ethyl acetate (3×5 mL). The filtrate was concentrated under reduced pressure. The residue was purified by silica gel column chromatography, eluted with 1%-25% ethyl acetate in petroleum ether to give 1-methyl-2-[2-methyl-4-[(4,4,5,5-tetramethyl-1,3,2-dioxaborolan-2-yl)methyl]phenyl]-4-(trifluoromethyl)imidazole (94 mg, 247.23 μmol, 25% yield) as a white solid. MS: m/z=381.35 [M+H]⁺.

### 1.3 2-[[5-(4-cyclopropyl-6-methoxy-pyrimidin-5-yl)-3-[[3-methyl-4-[1-methyl-4-(trifluoromethyl)imidazol-2-yl]phenyl]methyl]pyrazolo[4,3-d]pyrimidin-1-yl]methoxy]ethyl-trimethyl-silane

To a stirred solution of 1-methyl-2-[2-methyl-4-[(4,4,5,5-tetramethyl-1,3,2-dioxaborolan-2-yl)methyl]phenyl]-4-(trifluoromethyl)imidazole (94 mg, 247 μmol) and 2-[[3-bromo-5-(4-cyclopropyl-6-methoxy-pyrimidin-5-yl)pyrazolo[4,3-d]pyrimidin-1-yl]methoxy]ethyl-trimethyl-silane (148 mg, 310 μmol) in water (0.3 mL) and toluene (1.5 mL) was added bis(triphenylphosphine)palladium(II) chloride (19.8 mg, 28 μmol) and potassium phosphate (120 mg, 570 μmol) at 25° C. under nitrogen atmosphere. The resulting mixture was stirred for 16 hours at 90° C. under nitrogen atmosphere. The reaction was monitored by LCMS. The resulted mixture were filtered, the filter cake was washed with ethyl acetate (3×3 mL). The filtrate was concentrated under reduced pressure. The residue was purified by silica gel column chromatography, eluted with 1%-60% ethyl acetate in petroleum ether to give 2-[[5-(4-cyclopropyl-6-methoxy-pyrimidin-5-yl)-3-[[3-methyl-4-[1-methyl-4-(trifluoromethyl)imidazol-2-yl]phenyl]methyl]pyrazolo[4,3-d]pyrimidin-1-yl]methoxy]ethyl-trimethyl-silane (55 mg, 84.52 μmol, 33% yield) as a yellow solid. MS: m/z=651.55 [M+H]⁺.

### 1.4 5-(4-cyclopropyl-6-methoxy-pyrimidin-5-yl)-3-[[3-methyl-4-[1-methyl-4-(trifluoromethyl)imidazol-2-yl]phenyl]methyl]-1H-pyrazolo[4,3-d]pyrimidine

To a stirred solution of 2-[[5-(4-cyclopropyl-6-methoxy-pyrimidin-5-yl)-3-[[3-methyl-4-[1-methyl-4-(trifluoromethyl)imidazol-2-yl]phenyl]methyl]pyrazolo[4,3-d]pyrimidin-1-yl]methoxy]ethyl-trimethyl-silane (55 mg, 84 μmol) in dichloromethane (2 mL) was added trifluoroacetic acid (2.96 g, 25.96 mmol, 2 mL) at 25° C. under nitrogen atmosphere and then stirred at this temperature 1 hour. The resulted mixture was concentrated under reduced pressure. To a stirred solution of the residue in tetrahydrofuran (2 mL) was added ammonium hydroxide (28% aqueous solution, 2 mL) at 25° C. under nitrogen atmosphere and then stirred at this temperature 1 hour. The resulted mixture was concentrated under reduced pressure. The obtained product was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 95% B in 20 min; Detector: UV 254 & 210 nm; RT: 13 min. The collected fractions were combined and lyophilized to give 5-(4-cyclopropyl-6-methoxy-pyrimidin-5-yl)-3-[[3-methyl-4-[1-methyl-4-(trifluoromethyl)imidazol-2-yl]phenyl]methyl]-1H-pyrazolo[4,3-d]pyrimidine (11.5 mg, 22.09 μmol, 26% yield) as an off-white solid. MS: m/z=521.25 [M+H]⁺. ¹H NMR (400 MHz, Chloroform-d) δ 9.24 (s, 1H), 8.70 (s, 1H), 7.40-7.33 (m, 3H), 7.26 (d, J=7.8 Hz, 1H), 4.49 (s, 2H), 3.94 (s, 3H), 3.53 (s, 3H), 2.21 (s, 3H), 1.71-1.61 (m, 1H), 1.29-1.24 (m, 2H), 0.91-0.84 (m, 2H). ¹⁹F NMR (376 MHz, Chloroform) δ −62.45.

### Example 85. 4-chloro-2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine Compound 187

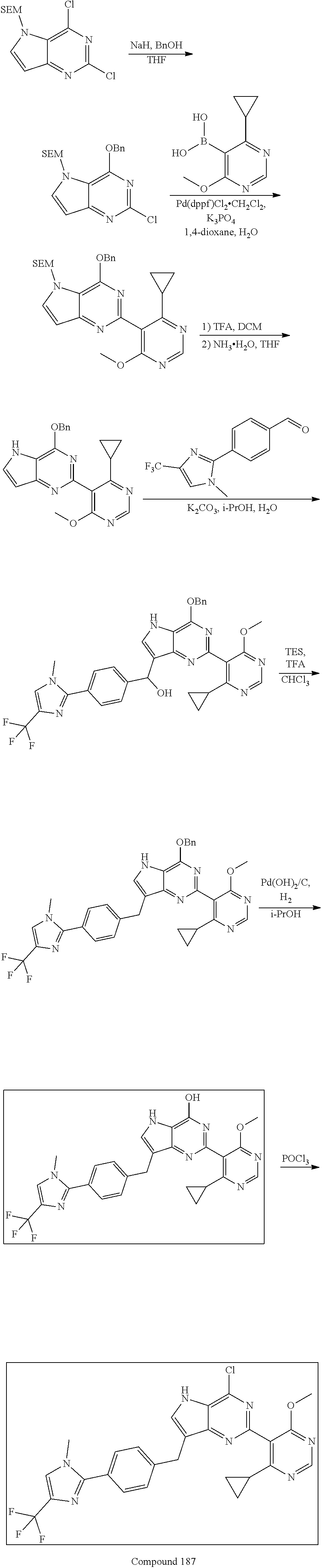

### 1.1 2-[(4-benzyloxy-2-chloro-pyrrolo[3,2-d]pyrimidin-5-yl)methoxy]ethyl-trimethyl-silane

To a solution of phenylmethanol (645 mg, 5.96 mmol, 617 μL) in THF (18 mL) was added sodium hydride (298 mg, 7.45 mmol, 60% dispersion in oil) in portions at 0° C. under nitrogen atmosphere. The reaction mixture was stirred at 0° C. for 30 min. Then 2-[(2-chloro-4-fluoro-pyrrolo[3,2-d]pyrimidin-5-yl)methoxy]ethyl-trimethyl-silane (1.5 g, 4.97 mmol) was added to the reaction mixture at 0° C., and stirred at 25° C. for 2 hours. The reaction was quenched with saturated ammonium chloride aqueous (200 mL), and then extracted with ethyl acetate (3×200 mL). The combined organic extracts were washed with brine (2×50 mL), dried over anhydrous sodium sulfate. After filtration, the filtrate was concentrated under reduced pressure. The residue was purified by chromatography on silica gel elute with 20%-40% ethyl acetate in petroleum ether to give 2-[(4-benzyloxy-2-chloro-pyrrolo[3,2-d]pyrimidin-5-yl)methoxy]ethyl-trimethyl-silane (1.5 g, 3.85 mmol, 77% yield) as a light yellow oil. MS: m/z=390.30, 392.30 [M+H]⁺. ¹H NMR (400 MHz, Chloroform-d) δ 7.57-7.49 (m, 2H), 7.48-7.33 (m, 4H) 6.63 (d, J=3.2 Hz, 1H), 5.64 (s, 2H), 5.61 (s, 2H), 3.47-3.41 (m, 2H), 0.85-0.81 (m, 2H), −0.07 (s, 9H).

### 1.2 2-[[4-benzyloxy-2-(4-cyclopropyl-6-methoxy-pyrimidin-5-yl)pyrrolo[3,2-d]pyrimidin-5-yl]methoxy]ethyl-trimethyl-silane

To a solution of 2-[(4-benzyloxy-2-chloro-pyrrolo[3,2-d]pyrimidin-5-yl)methoxy]ethyl-trimethyl-silane (400 mg, 1.03 mmol) and (4-cyclopropyl-6-methoxy-pyrimidin-5-yl)boronic acid (199 mg, 1.03 mmol) in 1,4-dioxane (5 mL) and water (1 mL) were added cyclopentyl(diphenyl)phosphane dichloromethane dichloropalladium iron (84 mg, 103 μmol) and potassium phosphate (653 mg, 3.08 mmol) at 25° C. under nitrogen atmosphere. The reaction mixture was stirred at 100° C. for 16 hours then cooled to room temperature and the solid was filtered out. The filtrate was concentrated under reduced pressure. The residue was purified by chromatography on silica gel eluted with 30%-40% ethyl acetate in petroleum ether to afford 2-[[4-benzyloxy-2-(4-cyclopropyl-6-methoxy-pyrimidin-5-yl)pyrrolo[3,2-d]pyrimidin-5-yl]methoxy]ethyl-trimethyl-silane (220 mg, 436.80 μmol, 43% yield) as a light-yellow solid. MS: m/z=504.15 [M+H]⁺. ¹H NMR (400 MHz, Chloroform-d) δ 8.67 (s, 1H), 7.56-7.46 (m, 3H), 7.44-7.32 (m, 3H), 6.77 (d, J=3.2 Hz, 1H), 5.71 (s, 2H), 5.67 (s, 2H), 3.95 (s, 3H), 3.58-3.49 (m, 2H), 1.80-1.73 (m, 1H), 1.25-1.19 (m, 2H), 0.90-0.85 (m, 4H), −0.05 (s, 9H).

### 1.3 4-benzyloxy-2-(4-cyclopropyl-6-methoxy-pyrimidin-5-yl)-5H-pyrrolo[3,2-d]pyrimidine

To a solution of 2-[[4-benzyloxy-2-(4-cyclopropyl-6-methoxy-pyrimidin-5-yl)pyrrolo[3,2-d]pyrimidin-5-yl]methoxy]ethyl-trimethyl-silane (219 mg, 434.81 μmol) in DCM (2 mL) was added 2,2,2-trifluoroacetic acid (2 mL) at 0° C. under nitrogen atmosphere. After the reaction mixture was stirred at 25° C. for 2 hours, it was concentrated under reduced pressure. The residue was added ammonium hydroxide (2 mL) and THF (2 mL). The resulting mixture was stirred at 25° C. for 1 hour then concentrated under reduced pressure. The residue was purified by chromatography on silica gel eluted with 40%-60% ethyl acetate in petroleum ether to give 4-benzyloxy-2-(4-cyclopropyl-6-methoxy-pyrimidin-5-yl)-5H-pyrrolo[3,2-d]pyrimidine (125 mg, 335 μmol, 77% yield) as a light-yellow solid. MS: m/z=374.30 [M+H]⁺. ¹H NMR (400 MHz, Chloroform-d) δ 8.93 (s, 1H), 8.67 (s, 1H), 7.51-7.46 (m, 3H), 7.42-7.38 (m, 3H), 6.79-6.77 (m, 1H), 5.64 (s, 2H), 3.94 (s, 3H), 1.80-1.74 (m, 1H), 1.25-1.20 (m, 2H), 0.94-0.82 (m, 2H).

### 1.4 [4-benzyloxy-2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol

To a solution of 4-benzyloxy-2-(4-cyclopropyl-6-methoxy-pyrimidin-5-yl)-5H-pyrrolo[3,2-d]pyrimidine (100 mg, 268 μmol) and 4-[1-methyl-4-(trifluoromethyl)imidazol-2-yl]benzaldehyde (68 mg, 268 μmol) in isopropyl alcohol (1 mL) were added potassium carbonate (44 mg, 321 μmol) and water (1 mL) at 0° C. under nitrogen atmosphere. The reaction mixture was stirred at 60° C. for 16 hours then cooled to 25° C., diluted with water (20 ml), extracted with ethyl acetate (3×20 ml). The combined organic layers were dried over anhydrous sodium sulfate and filtered. The filtrate was concentrated under reduced pressure. The residue was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 5% B to 5% B in 2 min, 5% B to 56% B in 19 min, 56% B to 56% B in 2 min, 56% B to 95% B in 10 min, 95% B to 95% B in 2 min; Detector: UV 254 & 210 nm; RT: 22 min. The collected fractions were combined, concentrated and then lyophilized overnight to give [4-benzyloxy-2-(4-cyclopropyl-6-methoxy-pyrimidin-5-yl)-5H-pyrrolo[3,2<d]pyrimidin-7-yl]-[4-[1-methyl-4-(trifluoromethyl)imidazol-2-yl]phenyl]methanol (25 mg, 39.83 μmol, 15% yield) as an off-white solid. MS: m/z=628.45. [M+H]⁺. ¹H NMR (400 MHz, Chloroform-d) δ 8.94 (s, 1H), 8.68 (s, 1H), 7.66-7.60 (m, 4H), 7.49-7.47 (m, 2H), 7.44-7.35 (m, 3H), 7.33 (s, 1H), 6.84 (s, 1H), 6.37 (s, 1H), 5.64 (s, 2H), 3.96 (s, 3H), 3.77 (s, 3H), 1.90-1.75 (m, 1H), 1.25-1.20 (m, 2H), 0.90-0.80 (m, 2H). ¹⁹F NMR (376 MHz, Chloroform-d) δ −62.69.

### 1.5 4-benzyloxy-2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine

To a solution of [4-benzyloxy-2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol (25 mg, 40 μmol) and triethylsilane (0.2 mL) in chloroform (0.2 mL) under nitrogen atmosphere was added trifluoroacetic acid (0.1 mL) at 0° C. The reaction mixture was stirred at 25° C. for 3 hours and then concentrated under reduced pressure. The residue was purified by Prep-TLC, eluted with 10% methanol in dichloromethane to afford 4-benzyloxy-2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (18 mg, 29.43 μmol, 74% yield) as an off-white solid. MS: m/z=612.40 [M+H]⁺. ¹H NMR (400 MHz, Chloroform-d) δ 12.12 (s, 1H), 8.74 (s, 1H), 7.51-7.45 (m, 2H), 7.41-7.31 (m, 6H), 7.16 (d, J=7.2 Hz, 2H), 6.96 (s, 1H), 5.74 (s, 2H), 4.22 (s, 2H), 3.94 (s, 3H), 3.68 (s, 3H), 1.35-1.20 (m, 3H), 0.90-0.80 (m, 2H). ¹⁹F NMR (376 MHz, Chloroform-d) δ −62.23, −75.95.

### 1.6 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-4-ol

To a solution of 4-benzyloxy-2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (18 mg, 29 μmol) in isopropyl alcohol (2 mL) was added palladium hydroxide carbon (18 mg, 20% purity) at 25° C., and then stirred at 25° C. for 2 hours under hydrogen. The resulted mixture was diluted with ethyl acetate (5 mL). The solid was filtered out and the filtrate was concentrated under reduced pressure. The residue was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 25 mL/min; Gradient: 5% B to 5% B in 5 min, 5% B to 46% B in 15 min, 46% B to 46% B in 2 min, 46% B to 95% B in 20 min; Detector: UV 254 & 210 nm; RT: 21 min. The collected fractions were combined, concentrated and then lyophilized overnight to give 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-4-ol (6 mg, 11.51 μmol, 40% yield) as an off-white solid. MS: m/z=522.25 [M+H]⁺. ¹H NMR (400 MHz, Methanol-d₄) δ 8.66 (s, 1H), 7.69 (s, 1H), 7.56 (d, J=8.0 Hz, 2H), 7.48 (d, J=8.4 Hz, 2H), 7.28 (s, 1H), 4.18 (s, 2H), 4.01 (s, 3H), 3.77 (s, 3H), 1.93-1.87 (m, 1H), 1.22-1.17 (m, 2H), 1.05-0.96 (m, 2H). ¹⁹F NMR (376 MHz, Chloroform-d) δ −63.92.

### 1.7 4-chloro-2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine

A solution of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-4-ol (4.3 mg, 8.25 μmol) in phosphoryl trichloride (0.5 mL) was stirred at 80° C. for 50 min under nitrogen atmosphere. The mixture was cooled down to 25° C. then concentrated under reduced pressure. The residue was dissolved in ethyl acetate (5 mL), then was added to cold saturated sodium bicarbonate aqueous solution (5 mL). The resulting mixture was extracted with ethyl acetate (3×5 mL). The combined organic layers were washed with brine (5 mL), dried over anhydrous sodium sulfate and filtered. The filtrate was concentrated under reduced pressure. The residue was purified by RP-Flash with the following conditions: Column: C18 spherical Column, 20-35 um, 100A, 20 g; Mobile Phase A: Water (5 mM aq. NH₄HCO₃), Mobile Phase B: MeCN: Flow rate: 25 mL/min; Gradient: 5% B to 5% B in 5 min, 5% B to 50% B in 13 min, 50% B to 50% B in 2 min, 50% B to 95% B in 15 min; Detector: UV 254 & 210 nm; RT: 19 min. The collected fractions were combined, concentrated and then lyophilized overnight to give 4-chloro-2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (1.1 mg, 2.04 μmol, 25% yield) as an off-white solid. MS: m/z=540.25, 542.20 [M+H]⁺. ¹H NMR (400 MHz, Chloroform-d) δ 8.93 (s, 1H), 8.68 (d, J=6.0 Hz, 1H), 7.58 (d, J=8.0 Hz, 2H), 7.46 (d, J=8.0 Hz, 2H), 7.34 (s, 1H), 7.30-7.25 (m, 1H), 4.29 (s, 2H), 3.94 (s, 3H), 3.78 (s, 3H), 1.74-1.67 (m, 1H), 1.27-1.22 (m, 2H), 0.92-0.87 (m, 2H). ¹⁹F NMR (376 MHz, Chloroform-d) δ −62.65.

### Example 86. 5-(4-cyclopropyl-6-methoxy-pyrimidin-5-yl)-3-[[2-methyl-4-[1-methyl-4-(trifluoromethyl)imidazol-2-yl]phenyl]methyl]-1H-pyrazolo[4,3-c]pyrimidine Compound

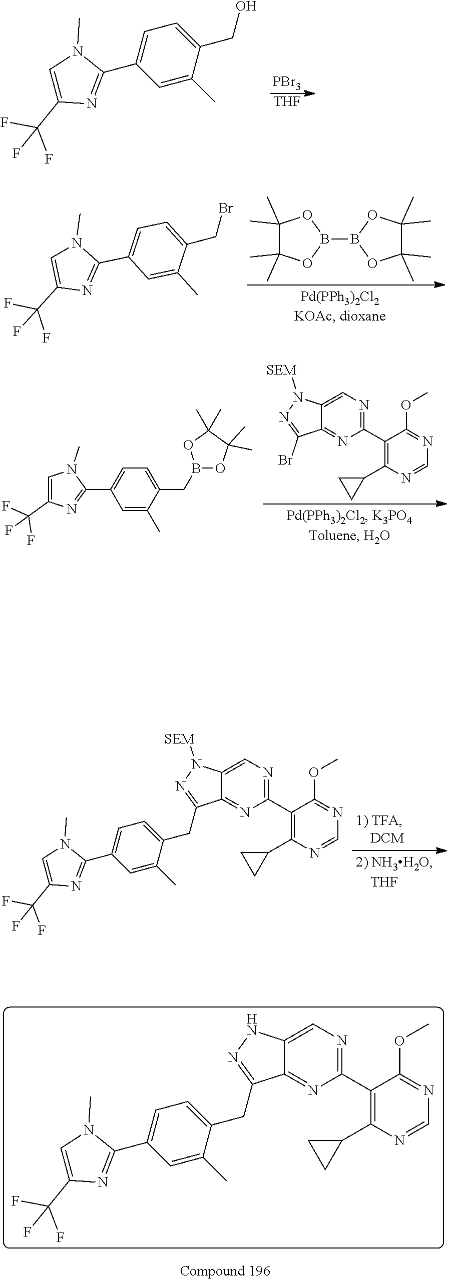

### 1.1 2-[4-(bromomethyl)-3-methyl-phenyl]-1-methyl-4-(trifluoromethyl)imidazole

To a solution of [2-methyl-4-[1-methyl-4-(trifluoromethyl)imidazol-2-yl]phenyl]methanol (250 mg, 925 μmol) in tetrahydrofuran (4 mL) were added tribromophosphane (751 mg, 2.78 mmol, 261 μL) at 0° C. The resulted solution was stirred at 25° C. for 2 hours. The reaction was quenched with saturated sodium bicarbonate aqueous solution (50 mL). The resulting mixture was extracted with ethyl acetate (50 mL), washed with brine (3×20 mL). The organic layer was dried over anhydrous sodium sulfate. After filtration, the filtrate was concentrated under reduced pressure. The residue was purified by silica gel column chromatography, eluted with 1%-30% ethyl acetate in petroleum ether to give 2-[4-(bromomethyl)-3-methyl-phenyl]-1-methyl-4-(trifluoromethyl)imidazole (200 mg, 600.34 μmol, 65% yield) as a yellow solid. MS: m/z=333.10, 335.10 [M+H]⁺.

### 1.2 1-methyl-2-[3-methyl-4-[(4,4,5,5-tetramethyl-1,3,2-dioxaborolan-2-yl)methyl]phenyl]-4-(trifluoromethyl)imidazole

To a solution of 2-[4-(bromomethyl)-3-methyl-phenyl]-1-methyl-4-(trifluoromethyl)imidazole (370 mg, 1.11 mmol) in 1,4-dioxane (10 mL) were added 4,4,5,5-tetramethyl-2-(4,4,5,5-tetramethyl-1,3,2-dioxaborolan-2-yl)-1,3,2-dioxaborolane (423 mg, 1.67 mmol), potassium acetate (218 mg, 2.22 mmol) and bis(triphenylphosphine)palladium(II) chloride (78 mg, 111 μmol) at 25° C. The resulted solution was stirred at 80° C. for 16 hours. The resulting mixture was diluted with ethyl acetate (100 mL), washed with brine (3×30 mL). The organic layer was dried over anhydrous sodium sulfate. After filtration, the filtrate was concentrated under reduced pressure. The residue was purified by silica gel column chromatography, eluted with 1%-20% ethyl acetate in petroleum ether to give 1-methyl-2-[3-methyl-4-[(4,4,5,5-tetramethyl-1,3,2-dioxaborolan-2-yl)methyl]phenyl]-4-(trifluoromethyl)imidazole (320 mg, 841.64 μmol, 76% yield) as a yellow solid. MS: m/z=381.10 [M+H]⁺. ¹H NMR (400 MHz, Chloroform-d) δ 7.44 (s, 1H), 7.32-7.29 (m, 2H), 7.24 (d, J=8.0 Hz, 1H), 3.77 (s, 3H), 2.34 (s, 3H) 2.32 (s, 2H), 1.25 (m, 12H). ¹⁹F NMR (376 MHz, Chloroform-d) δ −62.63.

### 1.3 2-[[5-(4-cyclopropyl-6-methoxy-pyrimidin-5-yl)-3-[[2-methyl-4-[1-methyl-4-(trifluoromethyl)imidazol-2-yl]phenyl]methyl]pyrazolo[4,3-d]pyrimidin-1-yl]methoxy]ethyl-trimethyl-silane

To a solution of 1-methyl-2-[3-methyl-4-[(4,4,5,5-tetramethyl-1,3,2-dioxaborolan-2-yl)methyl]phenyl]-4-(trifluoromethyl)imidazole (180 mg, 473 μmol) and 2-[[3-bromo-5-(4-cyclopropyl-6-methoxy-pyrimidin-5-yl)pyrazolo[4,3-d]pyrimidin-1-yl]methoxy]ethyl-trimethyl-silane (226 mg, 473 μmol) in water (1 mL) and toluene (5 mL) were added bis(triphenylphosphine)palladium(II) chloride (100 mg, 142 μmol) and potassium phosphate (301 mg, 1.42 mmol) at 25° C. The resulted solution was stirred at 90° C. for 16 hours. The resulting mixture was diluted with ethyl acetate (100 mL), washed with brine (3×30 mL). The organic layer was dried over anhydrous sodium sulfate. After filtration, the filtrate was concentrated under reduced pressure. The residue was purified by silica gel column chromatography, eluted with 1%-80% ethyl acetate in petroleum ether to give 2-[[5-(4-cyclopropyl-6-methoxy-pyrimidin-5-yl)-3-[[2-methyl-4-[1-methyl-4-(trifluoromethyl)imidazol-2-yl]phenyl]methyl]pyrazolo[4,3-d]pyrimidin-1-yl]methoxy]ethyl-trimethyl-silane (40 mg, 61.47 μmol, 13% yield) as a yellow solid. MS: m/z=651.55 [M+H]⁺. ¹H NMR (400 MHz, Chloroform-d) δ 9.36 (s, 1H), 8.70 (s, 1H), 7.49 (s, 1H), 7.47-7.43 (m, 1H), 7.34-7.29 (m, 2H), 5.80 (s, 2H), 4.50 (s, 2H), 3.94 (s, 3H), 3.75 (s, 3H), 3.67-3.60 (m, 2H), 2.54 (s, 3H), 1.78-1.56 (m, 1H), 1.02-0.80 (m, 6H), −0.02 (s, 9H). ¹⁹F NMR (376 MHz, Chloroform-d) δ −62.52.

### 1.4 5-(4-cyclopropyl-6-methoxy-pyrimidin-5-yl)-3-[[2-methyl-4-[1-methyl-4-(trifluoromethyl)imidazol-2-yl]phenyl]methyl]-1H-pyrazolo[4,3-d]pyrimidine

A solution of 2-[[5-(4-cyclopropyl-6-methoxy-pyrimidin-5-yl)-3-[[2-methyl-4-[1-methyl-4-(trifluoromethyl)imidazol-2-yl]phenyl]methyl]pyrazolo[4,3<d]pyrimidin-1-yl]methoxy]ethyl-trimethyl-silane (30 mg, 46 μmol) in dichloromethane (0.5 mL) and trifluoroacetic acid (0.5 mL) was stirred at 25° C. for 1 hour then was concentrated under reduced pressure. The residue was dissolved in tetrahydrofuran (0.5 mL) and then was added ammonium hydroxide (0.5 mL) and then stirred at 25° C. for another 30 min. The resulted mixture was concentrated under reduced pressure. The residue was purified by RP-Flash with the following conditions: Column: Spherical C18, 20-40 um, 40 g; Mobile Phase A: Water (5 mM aq. NH₄HCO₃), Mobile Phase B: MeCN; Flow rate: 40 mL/min; Gradient (B %): 2% B to 2% B in 5 min, 2% B to 62% B in 15 min, 62% B to 62% B in 3 min, 62% B to 95% B in 10 min, Detector: UV 254 & 210 Din; RT: 21 min. The collected fractions were combined and concentrated under reduced pressure and then lyophilized to give 5-(4-cyclopropyl-6-methoxy-pyrimidin-5-yl)-3-[[2-methyl-4-[1-methyl-4-(trifluoromethyl)imidazol-2-yl]phenyl]methyl]-1H-pyrazolo[4,3-d]pyrimidine as an off-white solid. MS: m/z=521.25 [M+H]⁺. ¹H NMR (400 MHz, Chloroform-d) δ 9.26 (s, 1H), 8.70 (s, 1H), 7.54 (s, 1H), 7.45 (d, J=8.0 Hz, 1H), 7.40-7.31 (m, 2H), 4.52 (s, 2H), 3.97 (s, 3H), 3.80 (s, 3H), 2.56 (s, 3H), 1.69-1.66 (m, 1H), 1.34-1.24 (m, 2H), 0.91-0.85 (m, 2H). ¹⁹F NMR (376 MHz, Chloroform-d) δ −62.44.

### Example 87. 2-[4-(1-fluorocyclopropyl)-6-methoxy-pyrimidin-5-yl]-7-[[4-[1-methyl-4-(trifluoromethyl)imidazol-2-yl]phenyl]methyl]-5H-pyrrolo[3,2-d]pyrimidine Compound 8

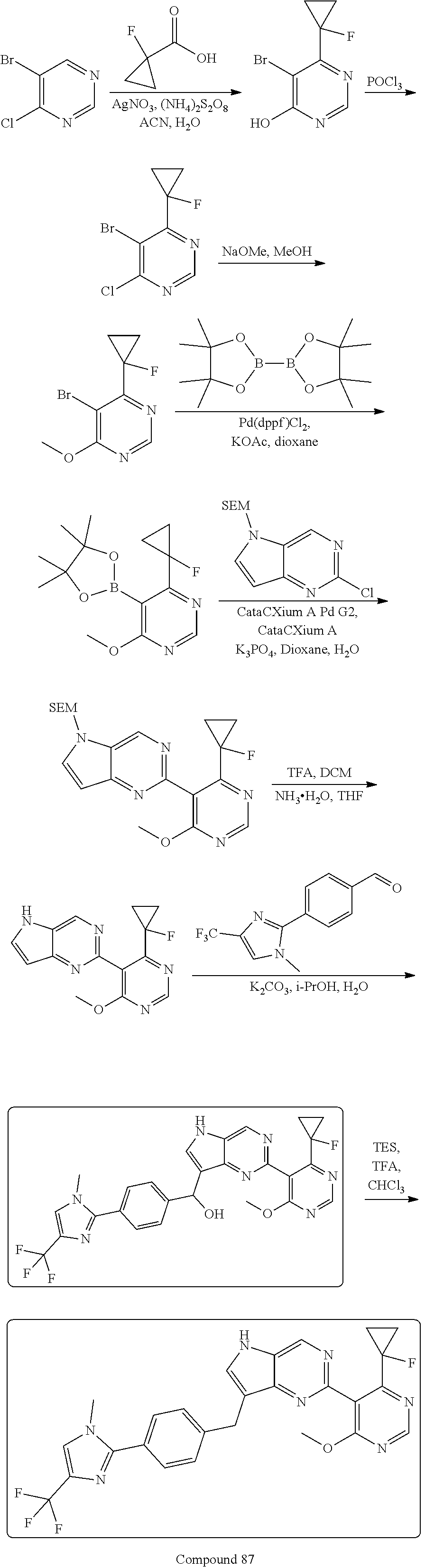

### 1.1 5-bromo-6-(1-fluorocyclopropyl)pyrimidin-4-ol

To a solution of 5-bromo-4-chloro-pyrimidine (4 g, 20.68 mmol) in acetonitrile (60 mL) and water (45 mL) were added 1-fluorocyclopropanecarboxylic acid (6.46 g, 62.04 mmol) and silver nitrate (7.03 g, 41.36 mmol) at 25° C. under nitrogen atmosphere. The reaction mixture was degassed with nitrogen. The reaction mixture was heat up to 80 TC. A solution of ammonium persulfate (9.44 g, 41.36 mmol, dissolved in 15 mL of water) was then added dropwise over 5 min. The resulting mixture was stirred at 80° C. for 16 hours. The reaction mixture was cooled down to room temperature, then diluted with water (100 mL), extracted with ethyl acetate (3×50 mL). The combined organic layers were washed with brine (50 mL), dried over anhydrous sodium sulfate. After filtration, the filtrate was concentrated under reduced pressure. The resulted residue was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 30% B in 8 min, 30% B to 30% B in 4 min, 30% B to 95% B in 8 min; Detector: UV 254 & 210 nm; RT: 16 min. The collected fractions were combined, concentrated and then lyophilized to give 5-bromo-6-(1-fluorocyclopropyl)pyrimidin-4-ol (1.1 g, 4.72 mmol, 23% yield) as an off-white solid. MS: m/z=232.95, 234.95 [M+H]⁺. ¹H NMR (400 MHz, Chloroform-d) δ 13.00 (s, 1H), 8.13 (s, 1H), 1.59-1.51 (m, 2H), 1.49-1.45 (m, 2H).

### 1.2 5-bromo-4-chloro-6-(1-fluorocyclopropyl)pyrimidine

To a solution of 5-bromo-6-(1-fluorocyclopropyl)pyrimidin-4-ol (1.1 g, 4.72 mmol) in phosphorus oxychloride (15 mL) was stirred at 80° C. for 16 hours under nitrogen atmosphere. The reaction solution was detected by LCMS. The resulted solution was concentrated under reduced pressure. The residue was diluted with ethyl acetate (100 mL), washed by saturated sodium bicarbonate aqueous solution (50 mL×2), dried over anhydrous sodium sulfate. After filtration, the filtrate was concentrated under reduced pressure to give crude product 5-bromo-4-chloro-6-(l-fluorocyclopropyl)pyrimidine (1 g, 3.98 mmol, 84% yield) as a yellow solid. MS: m/z=250.90, 252.90 [M+H]+.

### 1.3 5-bromo-4-(1-fluorocyclopropyl)-6-methoxy-pyrimidine

To a solution of 5-bromo-4-chloro-6-(1-fluorocyclopropyl)pyrimidine (1 g, 3.98 mmol) in methanol (10 mL) was added sodium methoxide (1.43 g, 7.95 mmol, 30% in methanol) at 25° C., The resulted solution was stirred at 40° C. for 2 hours under nitrogen atmosphere. The reaction was detected by LCMS. The resulted mixture was quenched by saturated ammonium chloride aqueous solution (20 mL), extracted with ethyl acetate (3×20 mL), dried over anhydrous sodium sulfate. After filtration, the filtrate was concentrated under reduced pressure. The residue was purified by silica gel column chromatography, eluted with 15%-17% ethyl acetate in petroleum ether. The collected fractions were combined and concentrated under reduced pressure to give 5-bromo-4-(1-fluorocyclopropyl)-6-methoxy-pyrimidine (900 mg, 3.64 mmol, 91% yield) as an off-white solid. MS: m/z=246.95, 248.95 [M+H]⁺. ¹H NMR (400 MHz, Chloroform-d) δ 8.58 (s, 1H), 4.10 (s, 3H), 1.60-1.32 (m, 4H).

### 1.4 4-(1-fluorocyclopropyl)-6-methoxy-5-(4,4,5,5-tetramethyl-1,3,2-dioxaborolan-2-yl)pyrimidine

To a stirred mixture of 5-bromo-4-(1-fluorocyclopropyl)-6-methoxy-pyrimidine (900 mg, 3.64 mmol) and potassium acetate (715 mg, 7.29 mmol) in 1,4-dioxane (15 mL) was added Bis(pinacolato)diboron (1.39 g, 5.46 mmol) and 1,1′-bis(diphenylphosphino)ferrocene-palladium(II)dichloride dichloromethane complex (149 mg, 182 μmol) under nitrogen atmosphere at 25° C. The resulting mixture was stirred at 80° C. for 16 hours. The reaction was cooled down to room temperature, then quenched with water (50 mL), extracted with dichloromethane (3×20 mL). The combined organic layers were washed with brine (40 mL), dried over anhydrous sodium sulfate. After filtration, the filtrate was concentrated under reduced pressure. The resulted residue was purified by RP-Flash with the following conditions: Column: C18 spherical Column, 20-35 um, 100A, 80 g; Mobile Phase A: Water (5 mM aq. NH₄HCO₃), Mobile Phase B: MeCN; Flow rate: 60 mL/min; Gradient: 5% B to 5% B in 5 min, 5% B to 63% B in 10 min, 63% B to 63% B in 5 min, 63% B to 95% B in 5 min; Detector: UV 254& 210 nm; RT: 18 min. The collected fractions were combined, concentrated and then lyophilized to give 4-(1-fluorocyclopropyl)-6-methoxy-5-(4,4,5,5-tetramethyl-1,3,2-dioxaborolan-2-yl)pyrimidine (430 mg, 1.46 mmol, 40% yield) as an off-white solid. MS: m/z=295.10 [M+H]⁺. ¹H NMR (400 MHz, Chloroform-d) δ 8.60 (s, 1H), 3.99 (s, 3H), 1.54-1.53 (m, 2H), 1.51-1.49 (m, 2H), 1.28 (s, 12H).

### 1.5 2-[[2-[4-(1-fluorocyclopropyl)-6-methoxy-pyrimidin-5-yl]pyrrolo[3,2-d]pyrimidin-5-yl]methoxy]ethyl-trimethyl-silane

To a stirred mixture of 4-(1-fluorocyclopropyl)-6-methoxy-5-(4,4,5,5-tetramethyl-1,3,2-dioxaborolan-2-yl)pyrimidine (400 mg, 1.36 mmol) and 2-[(2-chloropyrrolo[3,2-d]pyrimidin-5-yl)methoxy]ethyl-trimethyl-silane (772 mg, 2.72 mmol) in 1,4-dioxane (5 mL) and water (1 mL) were added bis(adamant-1-yl)(butyl)phosphine (49 mg, 136 μmol), chloro[(di(1-adamantyl)-n-butylphosphine)-2-(2-aminobiphenyl)]palladium(II) (91 mg, 136 μmol) and potassium phosphate (577 mg, 2.72 mmol) under nitrogen atmosphere at 25° C. The resulting mixture was stirred at 90° C. for 16 hours. The reaction was cooled to room temperature, then diluted with water (10 mL), extracted with dichloromethane (3×10 mL). The combined organic layers were washed with brine (10 mL), dried over anhydrous sodium sulfate. After filtration, the filtrate was concentrated under reduced pressure. The resulted residue was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 52% B in 8 min, 52% B to 52% B in 4 min, 52% B to 95% B in 8 min; Detector: UV 254 & 210 nm: RT: 18 min. The collected fractions were combined, concentrated and then lyophilized to give 2-[[2-[4-(1-fluorocyclopropyl)-6-methoxy-pyrimidin-5-yl]pyrrolo[3,2-d]pyrimidin-5-yl]methoxy]ethyl-trimethyl-silane (100 mg, 240.65 μmol, 18% yield) as an off-white solid. MS: m/z=416.15 [M+H]⁺. ¹H NMR (400 MHz, Chloroform-d) δ 9.05 (s, 1H), 8.67 (s, 1H), 7.60-7.52 (m, 1H), 6.76-6.69 (m, 1H), 5.56 (s, 2H), 3.92 (s, 3H), 3.56-3.46 (m, 2H), 1.58-1.54 (m, 2H), 1.42-1.24 (m, 2H), 0.95-0.88 (m, 2H), −0.04 (s, 9H).

### 1.6 2-[4-(1-fluorocyclopropyl)-6-methoxy-pyrimidin-5-yl]-5H-pyrrolo[3,2-d]pyrimidine

To a stirred solution of 2-[[2-[4-(1-fluorocyclopropyl)-6-methoxy-pyrimidin-5-yl]pyrrolo[3,2-d]pyrimidin-5-yl]methoxy]ethyl-trimethyl-silane (100 mg, 240.65 μmol) in methylene chloride (0 mL) was added trifluoroacetic acid (I mL) and then stirred for 2 hours at 25° C. The resulted mixture was concentrated under reduced pressure, then tetrahydrofuran (1 mL) and ammonia water (1 mL) were added in the above mixture, the resulted mixture was stirred for 30 min at 25° C. The resulted mixture was concentrated under reduced pressure. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 65% B in 20 min, 65% B to 65% B in 4 min, 65% B to 95% B in 5 min; Detector: UV 254& 210 nm; RT: 25 min. The collected fractions were combined, concentrated and then lyophilized to give 2-[4-(1-fluorocyclopropyl)-6-methoxy-pyrimidin-5-yl]-5H-pyrrolo[3,2-d]pyrimidine (50 mg, 175.27 μmol, 73% yield) as an off-white solid. MS: m/z=286.10 [M+H]⁺. ¹H NMR (400 MHz, Methanol-d₄) δ 8.94 (s, 1H), 8.72 (s, 1H), 7.93 (d, J=3.2 Hz, 1H), 6.68 (d, J=3.2 Hz, 1H), 3.94 (s, 3H), 1.62-1.50 (m, 2H), 1.39-1.27 (m, 2H).

### 1.7 [2-[4-(1-fluorocyclopropyl)-6-methoxy-pyrimidin-5-yl]-5H-pyrrolo[3,2-d]pyrimidin-7-yl]-[4-[1-methyl-4-(trifluoromethyl)imidazol-2-yl]phenyl]methanol

To a solution of 2-[4-(1-fluorocyclopropyl)-6-methoxy-pyrimidin-5-yl]-5H-pyrrolo[3,2-d]pyrimidine (50 mg, 175.27 μmol) in isopropyl alcohol (1 mL) and water (1 mL) were added 4-[1-methyl-4-(trifluoromethyl)imidazol-2-yl]benzaldehyde (45 mg, 175 μmol) and potassium carbonate (29 mg, 210 μmol) and then stirred at 40° C. for 24 hours under nitrogen atmosphere. The reaction solution was detected by TLC and LCMS. The resulted mixture was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 30% B in 10 min, 30% B to 30% B in 4 min, 30% B to 95% B in 8 min; Detector: UV 254 & 210 nm; RT: 19 min. The collected fractions were combined, concentrated and then lyophilized to give [2-[4-(1-fluorocyclopropyl)-6-methoxy-pyrimidin-5-yl]-5H-pyrrolo[3,2-d]pyrimidin-7-yl]-[4-[1-methyl-4-(trifluoromethyl)imidazol-2-yl]phenyl]methanol (40 mg, 37.07 μmol, 42% yield) as an off-white solid. MS: m/z=540.20 [M +H]⁺. ¹H NMR (400 MHz, Methanol-d₄) δ 8.91 (s, 1H), 8.72 (s, 11H), 7.76 (s, 1H), 7.71 (d, J=8.4 Hz, 3H), 7.63 (d, J=8.0 Hz, 2H), 6.41 (s, 1H), 3.94 (s, 3H), 3.78 (s, 3H), 1.57-1.45 (m, 2H), 1.38-1.24 (m, 2H). ¹⁹F NMR (376 MHz, Methanol-d₄) δ −63.93, −190.07.

### 1.8 2-[4-(1-fluorocyclopropyl)-6-methoxy-pyrimidin-5-yl]-7-[[4-[1-methyl-4-(trifluormethyl)imidazol-2-yl]phenyl]methyl]-5H-pyrrolo[3,2-d]pyrimidine

To a solution of [2-[4-(1-fluorocyclopropyl)-6-methoxy-pyrimidin-5-yl]-5H-pyrrolo[3,2-d]pyrimidin-7-yl]-[4-[1-methyl-4-(trifluoromethyl)imidazol-2-yl]phenyl]methanol (40 mg, 74 μmol) in trichloromethane (1 mL) was added triethyl-silane (1 mL) and trifluoroacetic acid (0.5 mL) at 25° C., then was stirred at 25° C. for 16 hours. The resulted solution was detected by LCMS, major desired product. The resulted solution was concentrated under reduced pressure. The residue was co-evaporated with toluene (three times, 5 mL each). The resulted residue was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 47% B in 10 min, 47% B to 47% B in 2 min, 47% B to 95% B in 5 min; Detector: UV 254 & 210 nm; RT: 17 min. The collected fractions were combined, concentrated and then lyophilized to give 2-[4-(1-fluorocyclopropyl)-6-methoxy-pyrimidin-5-yl]-7-[[4-[1-methyl-4-(trifluoromethyl)imidazol-2-yl]phenyl]methyl]-5H-pyrrolo[3,2-d]pyrimidine (24.5 mg, 46.80 μmol, 63% yield) as an off-white solid. MS: m/z=524.25 [M+H]⁺. ¹H NMR (400 MHz, Methanol-d₄) δ 8.91 (s, 11H), 8.72 (s, 1H), 7.72 (s, 11H), 7.68 (s, 1H), 7.57 (d, J=8.0 Hz, 2H), 7.50 (d, J=8.0 Hz, 2H), 4.29 (s, 2H), 3.95 (s, 3H), 3.77 (s, 3H), 1.57-1.50 (m, 2H), 1.37-1.24 (m, 2H). ¹⁹F NMR (376 MHz, Methanol-d4) 5-63.70, −63.93, −190.27, −190.31.

### Example 88. 2-(4-cyclopropyl-6-methoxy-pyrimidin-5-yl)-7-[rel-(1S)-1-[4-[1-methyl-4-(trifluoromethyl)imidazol-2-yl]phenyl]ethyl]-5H-pyrrolo[3,2-d]pyrimidine (Compound 192) & 2-(4-cyclopropyl-6-methoxy-pyrimidin-5-yl)-7-[rel-(1R)-1-[4-[1-methyl-4-(trifluoromethyl)imidazol-2-yl]phenyl]ethyl]-5H-pyrrolo[3,2-d]pyrimidine (Compound 89)

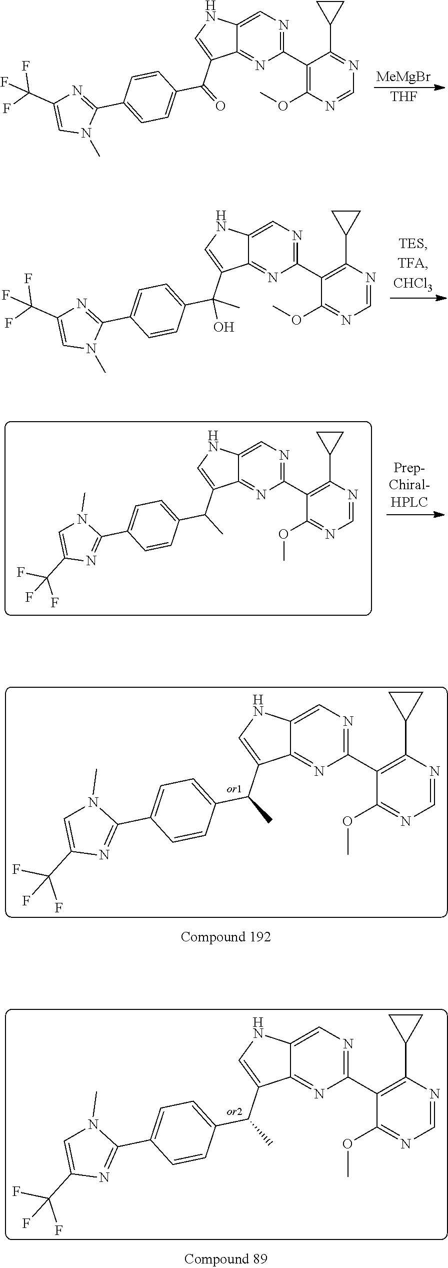

### 1.1 1-[2(4-cyclopropyl-6-methoxy-pyrimidin-5-yl)-5H-pyrrolo[3,2-d]pyrimidin-7-yl]-1-[4-[1-methyl-4-(trifluoromethyl)imidazol-2-yl]phenyl]ethanol

To a stirred mixture of [2-(4-cyclopropyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanone (850 mg, 1.64 mmol) in tetrahydrofuran (8 mL) was added methyl magnesium bromide (1 M solution in THF, 3.27 mmol, 3.27 mL) under nitrogen atmosphere at 0° C. The resulting mixture was stirred at 25° C. for 4 hours. The reaction was quenched by the addition of ammonia chloride aqueous solution (30 mL). The resulting mixture was extracted with ethyl acetate (3×30 mL). The combined organic layers were washed with brine (3×30 mL), dried over anhydrous sodium sulfate. After filtration, the filtrate was concentrated under reduced pressure. The obtained product was purified by reverse phase chromatography with the following conditions Column: C18 spherical Column, 20-35 um, 100A, 40 g; Mobile Phase A: Water (5 mM aq. NH₄HCO₃), Mobile Phase B: MeCN; Flow rate: 35 mL/min; Gradient: 5% B to 5% B in 5 min, 5% B to 55% B in 15 min, 55% B to 55% B in 4 min, 55% B to 95% B in 3 min; Detector: UV 254 & 210 nm; RT: 21.5 min. The product-containing fractions were collected and evaporated in vacuo and then lyophilized overnight to give 1-[2-(4-cyclopropyl-6-methoxy-pyrimidin-5-yl)-5H-pyrrolo[3,2-d]pyrimidin-7-yl]-1-[4-[1-methyl-4-(trifluoromethyl)imidazol-2-yl]phenyl]ethanol (400 mg, 746.94 μmol, 45% yield) as an off-white solid. MS: m/z=536.35 [M+H]⁺.

### 1.2 2-(4-cyclopropyl-6-methoxy-pyrimidin-5-yl)-7-[1-[4-[1-methyl-4-(trifluoromethyl)imidazol-2-yl]phenyl]ethyl]-5H-pyrrolo[3,2-d]pyrimidine

To a stirred mixture of 1-[2-(4-cyclopropyl-6-methoxy-pyrimidin-5-yl)-5H-pyrrolo[3,2-d]pyrimidin-7-yl]-1-[4-[1-methyl-4-(trifluoromethyl)imidazol-2-yl]phenyl]ethanol (400 mg, 746.94 μmol) in chloroform (4 mL) were added trifluoroacetic acid (2 mL) and triethoxysilane (4 mL) under nitrogen atmosphere at 25° C. The resulting mixture was stirred at 25° C. for 2 hours. The reaction was monitored by LCMS. The reaction mixture was concentrated under reduced pressure. The residue was purified by Prep-TLC, eluted with 6% methanol in dichloromethane to afford 70 mg crude product. The crude product was purified by reverse phase chromatography with the following conditions: Column: C18 spherical Column, 20-35 um, 100A, 20 g; Mobile Phase A: Water (5 mM aq. NH₄HCO₃), Mobile Phase B: MeCN; Flow rate: 25 mL/min; Gradient: 5% B to 5% B in 5 min, 5% B to 42% B in 7 min, 42% B to 42% B in 2 min, 42% B to 95% B in 10 min; Detector: UV 254 & 210 nm: RT: 14 min. The product-containing fractions were collected and evaporated in vacuo and then lyophilized overnight to give 2-(4-cyclopropyl-6-methoxy-pyrimidin-5-yl)-7-[1-[4-[1-methyl-4-(trifluoromethyl)imidazol-2-yl]phenyl]ethyl]-5H-pyrrolo[3,2-d]pyrimidine (130 mg, 250.23 μmol, 33% yield) as an off-white solid. MS: m/z=520.25 [M+H]⁺. ¹H NMR (400 MHz, Chloroform-d) δ 10.18 (s, 1H), 8.97 (s, 1H), 8.65 (s, 1H), 7.55 (d, J=8.4 Hz, 2H), 7.47 (d, J=8.0 Hz, 2H), 7.36 (s, 1H), 7.20 (s, 1H), 4.74-4.69 (m, 1H), 3.92 (s, 3H), 3.79 (s, 3H), 1.79-1.70 (m, 4H), 1.22-1.11 (m, 2H), 0.85-0.74 (m, 2H). ¹⁹F NMR (376 MHz, Chloroform-d) δ −62.524.

### 1.3 2-(4-cyclopropyl-6-methoxy-pyrimidin-5-yl)-7-[rel-(1S)-1-[4-[1-methyl-4-(trifluoromethyl)imidazol-2-yl]phenyl]ethyl]-5H-pyrrolo[3,2-c]pyrimidine (Compound 192) & 2-(4-cyclopropyl-6-methoxy-pyrimidin-5-yl)-7-[rel-(1R)-1-[4-[1-methyl-4-(trifluoromethyl)imidazol-2-yl]phenyl]ethyl]-5H-pyrrolo[3,2-d]pyrimidine (Compound 89)

2-(4-cyclopropyl-6-methoxy-pyrimidin-5-yl)-7-[1-[4-[1-methyl-4-(trifluoromethyl)imidazol-2-yl]phenyl]ethyl]-5H-pyrrolo[3,2-d]pyrimidine (130 mg, 250.23 μmol) was separated by Prep-Chiral-HPLC with the following conditions: Column: CHIRALPAK IE-3, 4.6×50 mm 3 um; Mobile Phase A: Hex (0.1% DEA): EtOH=75:25; Flow rate: 1 mL/min; Gradient: 0% B to 0% B; Injection Volume: 5 uL, Detector: UV 220/254 nm; RT1: 1.883 min, RT2: 2.297 min. The first product containing (RT1: 1.883 min) peak was combined, concentrated under reduced pressure and then lyophilized to give 2-(4-cyclopropyl-6-methoxy-pyrimidin-5-yl)-7-[rel-(1S)-1-[4-[1-methyl-4-(trifluoromethyl)imidazol-2-yl]phenyl]ethyl]-5H-pyrrolo[3,2-d]pyrimidine (47.5 mg, 91.43 μmol, 36% yield) (Compound 192, stereochemistry arbitrarily assigned) as an white solid. MS: m/z=520.25 [M+H]⁺. ¹H NMR (400 MHz, Chloroform-d) δ 10.12 (s, 1H), 9.05 (s, 1H), 8.67 (s, 1H), 7.57 (d, J=8.0 Hz, 1H), 7.48 (d, J=8.4 Hz, 2H), 7.35 (s, 1H), 7.31 (s, 1H), 4.77-4.71 (m, 1H), 3.94 (s, 3H), 3.79 (s, 3H), 1.82-1.72 (m, 4H), 1.25-1.16 (m, 2H), 0.87-0.83 (m, 2H). ¹⁹F NMR (376 MHz, Chloroform-d) δ −62.58.

The second product containing (RT2: 2.297 min) peak was combined, concentrated under reduced pressure and then lyophilized to give 2-(4-cyclopropyl-6-methoxy-pyrimidin-5-yl)-7-[rel-(1R)-1-[4-[1-methyl-4-(trifluoromethyl)imidazol-2-yl]phenyl]ethyl]-5H-pyrrolo[3,2-d]pyrimidine (49.4 mg, 95.09 μmol, 38% yield) (Compound 89, stereochemistry arbitrarily assigned) as an white solid. MS: m/z=520.25 [M+H]⁺. ¹H NMR (400 MHz, Chloroform-d) δ 10.12 (s, 1H), 9.03 (s, 1H), 8.66 (s, 1H), 7.56 (d, J=8.4 Hz, 2H), 7.48 (d, J=8.0 Hz, 2H), 7.36 (s, 1H), 7.25 (m, 1H), 4.77-4.71 (m, 1H), 3.93 (s, 3H), 3.79 (s, 3H), 1.83-1.69 (m, 4H), 1.25-1.15 (m, 2H), 0.88-0.76 (m, 2H). ¹⁹F NMR (376 MHz, Chloroform-d) δ −62.55.

### Example 89. 2-[4-(2,2-difluorocyclopropyl)-6-methoxy-pyrimidin-5-yl]-7-[[4-[1-methyl-4-(trifluoromethyl)imidazol-2-yl]phenyl]methyl]-5H-pyrrolo[3,2-d]pyrimidine Compound 147

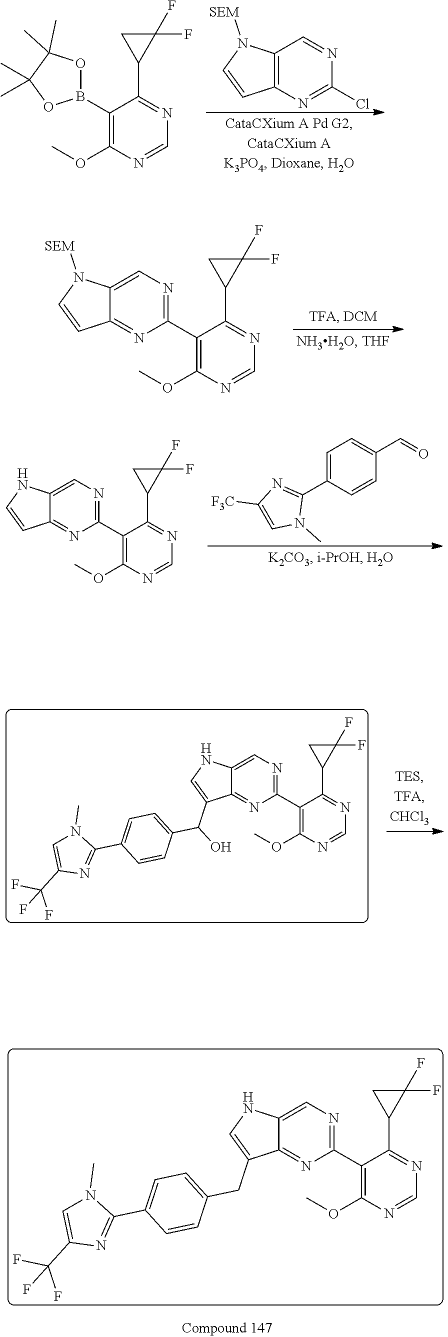

### 1.1 2-[[2-[4-(2,2-difluorocyclopropyl)-6-methoxy-yrimidin-5-yl]pyrrolo[3,2-d]pyrimidin-5-yl]methoxy]ethyl-trimethyl-silane

To a mixture of 4-(2,2-difluorocyclopropyl)-6-methoxy-5-(4,4,5,5-tetramethyl-1,3,2-dioxaborolan-2-yl)pyrimidine (470 mg, 1.51 mmol) and 2-[(2-chloropyrrolo[3,2-d]pyrimidin-5-yl)methoxy]ethyl-trimethyl-silane (855 mg, 3.01 mmol) in 1,4-dioxane (8.5 mL) and water (1.7 mL) under nitrogen atmosphere were added chloro[(diadamantan-1-yl)(n-butyl)phosphino][2-amino-1,1-biphenyl-2-yl]palladium(II) (101 mg, 151 μmol), butyldi-1-adamantylphosphine (54 mg, 151 μmol) and potassium phosphate (639 mg, 3.01 mmol) at 25° C. The resulted mixture was stirred at 90° C. for 16 hours. The reaction progress was monitored by LCMS. The resulting mixture was cooled down to room temperature and the solid was filtered out. The filtrate was concentrated under reduced pressure. The residue was purified by silica gel column chromatography, eluted with 1%-50% ethyl acetate in petroleum ether to give 2-[[2-[4-(2,2-difluorocyclopropyl)-6-methoxy-pyrimidin-5-yl]pyrrolo[3,2-d]pyrimidin-5-yl]methoxy]ethyl-trimethyl-silane (80 mg, 184.53 μmol, 12% yield) as a yellow solid. MS: m/z=434.20 [M+H]⁺. ¹H NMR (400 MHz, Chloroform-d) δ 9.16 (s, 1H), 8.80 (s, 1H), 7.67 (d, J=3.2 Hz, 1H), 6.82 (d, J=3.2 Hz, 1H), 5.62 (s, 2H), 3.98 (s, 3H), 3.61-3.55 (m, 2H), 2.71-2.63 (m, 1H), 2.52-2.45 (m, 1H), 1.76-1.59 (m, 1H), 1.04-0.92 (m, 2H), 0.01 (s, 9H). ¹⁹F NMR (376 MHz, Chloroform-d) δ −123.44, −123.84, −142.29, −142.69.

### 1.2 2-[4-(2,2-difluorocyclopropyl)-6-methoxy-pyrimidin-5-yl]-5H-pyrrolo[3,2-d]pyrimidine

To a solution of 2-[[2-[4-(2,2-difluorocyclopropyl)-6-methoxy-pyrimidin-5-yl]pyrrolo[3,2-d]pyrimidin-5-yl]methoxy]ethyl-trimethyl-silane (100 mg, 230 μmol) in dichloromethane (1 mL) was added trifluoroacetic acid (1.48 g, 12.98 mmol, 1 mL) at 25° C., and then stirred at this temperature for 2 hours. The reaction solution was concentrated under reduced pressure. The residue was dissolved in tetrahydrofuran (2 mL) and ammonium hydroxide (1 mL) was added at 0° C. The resulted mixture was stirred at 25° C. for 0.5 hour and then concentrated under reduced pressure. The residue was purified by Prep-TLC, eluted with 7% methanol in dichloromethane to give 2-[4-(2,2-difluorocyclopropyl)-6-methoxy-pyrimidin-5-yl]-5H-pyrrolo[3,2-d]pyrimidine (50 mg, 164.87 μmol, 71% yield) as a yellow solid. MS: m/z=304.10 [M+H]⁺. ¹H NMR (400 MHz, Chloroform-d) δ 9.46 (s, 1H), 9.07 (s, 1H), 8.79 (s, 1H), 7.72 (d, J=2.8 Hz, 1H), 6.85 (d, J=3.2 Hz, 1H), 3.96 (s, 3H), 2.73-2.65 (m, 1H), 2.53-2.45 (m, 1H), 1.74-1.65 (m, 1H).

### 1.3 [2-[4-(2,2-difluorocyclopropyl)-6-methoxy-pyrimidin-5-yl]-5H-pyrrolo[3,2-d]pyrimidin-7-yl]-[4-[1-methyl-4-(trifluoromethyl)imidazol-2-yl]phenyl]methanol

To a solution of 2-[4-(2,2-difluorocyclopropyl)-6-methoxy-pyrimidin-5-yl]-5H-pyrrolo[3,2-d]pyrimidine (30 mg, 99 μmol) in isopropyl alcohol (1 mL) and water (1 mL) were added 4-[1-methyl-4-(trifluoromethyl)imidazol-2-yl]benzaldehyde (25 mg, 99 μmol) and potassium carbonate (16 mg, 119 μmol) and then stirred for 24 hours at 40° C. The reaction solution was detected by TLC and LCMS. The resulted mixture was purified directly by RP-Flash with the following conditions: Column: C18 spherical Column, 20-35 um, 100A, 40 g; Mobile Phase A: Water (5 mM aq. NH₄HCO₁), Mobile Phase B: MeCN; Flow rate: 60 mL/min; Gradient: 5% B to 5% B in 5 min. 5% B to 30% B in 10 min, 30% B to 30% B in 4 min, 30% B to 95% B in 8 min; Detector: UV 254 & 210 nm: RT: 19 min. The collected fractions were combined, concentrated and then lyophilized to give [2-[4-(2,2-difluorocyclopropyl)-6-methoxy-pyrimidin-5-yl]-5H-pyrrolo[3,2-d]pyrimidin-7-yl]-[4-[1-methyl-4-(trifluoromethyl)imidazol-2-yl]phenyl]methanol (12 mg, 21.53 μmol, 22% yield) as an off-white solid. MS: m/z=558.20 [M +H]⁺. ¹H NMR (400 MHz, Chloroform-d) δ 9.27 (s, 1H), 9.03 (s, 1H), 8.81 (s, 1H), 7.67 (s, 4H), 7.37 (s, 1H), 7.16 (s, 1H), 6.43 (s, 1H), 4.01 (s, 3H), 3.80 (s, 3H), 2.65-2.52 (m, 2H), 1.39-1.37 (m, 1H). ¹⁹F NMR (376 MHz, Chloroform-d) δ −62.611, −123.745, −124.145, −141.963, −142.365.

### 2-[4-(2,2-difluorocyclopropyl)-6-methoxy-pyrimidin-5-yl]-7-[[4-[1-methyl-4-(trifluoromethyl)imidazol-2-yl]phenyl]methyl]-5H-pyrrolo[3,2-d]pyrimidine

To a solution of [2-[4-(2,2-difluorocyclopropyl)-6-methoxy-pyrimidin-5-yl]-5H-pyrrolo[3,2-d]pyrimidin-7-yl]-[4-[1-methyl-4-(trifluoromethyl)imidazol-2-yl]phenyl]methanol (10 mg, 18 μmol) in chloroform (1 mL) was added trifluoroacetic acid (0.5 mL) and triethyl-silane (1 mL) at 25° C., then was stirred at 25° C. for 16 hours. The resulted solution was detected by LCMS, major desired product. The resulted solution was concentrated under reduced pressure. The residue was co-evaporated with toluene (three times, 5 mL each). The resulted residue was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 47% B in 10 min, 47% B to 47% B in 2 min, 47% B to 95% B in 5 min; Detector: UV 254 & 210 nm; RT: 17 min. The collected fractions were combined, concentrated and then lyophilized to give 2-[4-(2,2-difluorocyclopropyl)-6-methoxy-pyrimidin-5-yl]-7-[[4-[1-methyl-4-(trifluoromethyl)imidazol-2-yl]phenyl]methyl]-5H-pyrrolo[3,2-d]pyrimidine (1.4 mg, 2.59 μmol, 14% yield) as an off-white solid. MS: m/z=542.20 [M+H]⁺. ¹H NMR (400 MHz, Chloroform-d) δ 9.58 (s, 1H), 9.00 (s, 1H), 8.79 (s, 1H), 7.55 (d, J=8.0 Hz, 2H), 7.43 (d, J=8.0 Hz, 2H), 7.35 (s, 1H), 7.25 (s, 1H), 4.36-4.22 (m, 2H), 3.98 (s, 3H), 3.77 (s, 3H), 2.70-2.62 (m, 1H), 2.52-2.46 (m, 1H), 1.30-1.26 (m, 1H). ¹⁹F NMR (376 MHz, Chloroform-d) δ −62.54, −123.58, −123.98, −142.11, −142.51.

### Example 90. 2-(4-cyclopropyl-6-methoxy-pyrimidin-5-yl)-7-[[4-[1-(trideuteriomethyl)-4-(trifluoromethyl)imidazol-2-yl]phenyl]methyl]-5H-pyrrolo[3,2-d]pyrimidine Compound 135

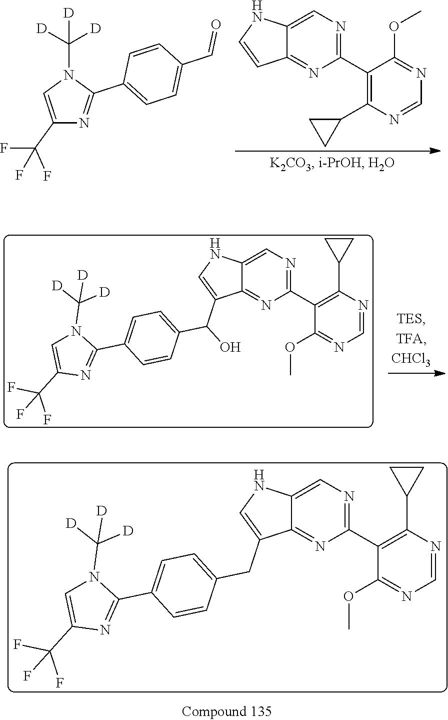

### 1.1 [2-(4-cyclopropyl-6-methoxy-pyrimidin-5-yl)-5H-pyrrolo[3,2-d]pyrimidin-7-yl]-[4-[1-(trideuteriomethyl)-4-(trifluoromethyl)imidazol-2-yl]phenyl]methanol

To a stirred solution of 4-[1-(trideuteriomethyl)-4-(trifluoromethyl)imidazol-2-yl]benzaldehyde (45 mg, 175 μmol) and 2-(4-cyclopropyl-6-methoxy-pyrimidin-5-yl)-5H-pyrrolo[3,2-d]pyrimidine (47 mg, 175 μmol) in water (0.5 mL) and isopropyl alcohol (0.5 mL) was added potassium carbonate (48 mg, 345 μmol) at 25° C. under nitrogen atmosphere. The resulting mixture was stirred for 16 hours at 40° C. under nitrogen atmosphere. The reaction was monitored by LCMS. The resulting mixture was diluted with water (10 mL). The resulting mixture was extracted with ethyl acetate (3×25 mL). The combined organic layers were washed with brine (10 mL), dried over anhydrous sodium sulfate. After filtration, the filtrate was concentrated under reduced pressure. The residue was purified by silica gel column chromatography, eluted with 50% ethyl acetate in petroleum ether to afford [2-(4-cyclopropyl-6-methoxy-pyrimidin-5-yl)-5H-pyrrolo[3,2-d]pyrimidin-7-yl]-[4-[1-(trideuteriomethyl)-4-(trifluoromethyl)imidazol-2-yl]phenyl]methanol (25 mg, 47.66 μmol, 27% yield) as a white solid. MS: m/z=525.25 [M+H]+. ¹H NMR (400 MHz, Chloroform-d) δ 9.24 (s, 1H), 8.71 (s, 1H), 7.71 (d, J=8.0 Hz, 2H), 7.66-7.60 (m, 3H), 7.49 (s, 1H), 6.46 (s, 1H), 4.00 (s, 3H), 1.90-1.85 (m, 1H), 1.29-1.25 (m, 2H), 0.98-0.89 (m, 2H), 19F NMR (376 MHz, Chloroform-d) δ −59.98.

### 1.2 2-(4-cyclopropyl-6-methoxy-pyrimidin-5-yl)-7-[[4-[1-(trideuteriomethyl)-4-(trifluoromethyl)imidazol-2-yl]phenyl]methyl]-5H-pyrrolo[3,2-d]pyrimidine

To a stirred solution of [2-(4-cyclopropyl-6-methoxy-pyrimidin-5-yl)-5H-pyrrolo[3,2-d]pyrimidin-7-yl]-[4-[1-(trideuteriomethyl)-4-(trifluoromethyl)imidazol-2-yl]phenyl]methanol (142 mg, 271 μmol) and triethylsilane (1.46 g, 12.52 mmol, 2 mL) in dichloromethane (2 mL) was added 2,2,2-trifluoroacetic acid (1.48 g, 12.98 mmol, 1 mL) at 25° C. under nitrogen atmosphere. The resulting mixture was stirred for 2 hours at 25° C. under nitrogen atmosphere. The resulting mixture % as concentrated under reduced pressure. The residue was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 95% B in 20 min; Detector: UV 254 & 210 nm; RT: 15.2 min. The collected fractions were combined, concentrated under reduced pressure and lyophilized to give 2-(4-cyclopropyl-6-methoxy-pyrimidin-5-yl)-7-[[4-[1-(trideuteriomethyl)-4-(trifluoromethyl)imidazol-2-yl]phenyl]methyl]-5H-pyrrolo[3,2-d]pyrimidine (78 mg, 153.39 μmol, 56% yield) as a white solid. MS: m/z=509.20 [M+H]⁺. ¹H NMR (400 MHz, Chloroform-d) δ 9.11 (s, 1H), 8.68 (s, 1H), 7.56 (d, J=8.0 Hz, 2H), 7.45 (d, J=8.0 Hz, 2H), 7.39-7.32 (m, 2H), 4.30 (s, 2H), 3.96 (s, 3H), 1.83-1.75 (m, 1H), 1.27-1.21 (m, 2H), 0.90-0.85 (m, 2H). ¹⁹F NMR (376 MHz, Chloroform-d) δ −62.56.

### Example 91. 5-(4-cyclopropyl-6-(difluoromethoxy)pyrimidin-5-yl)-3-(4-(1-isopropyl-4-(trifluoromethyl)-1H-imidazol-2-yl)benzyl)-1H-pyrazolo[4,3-d]yrimidine Compound 137

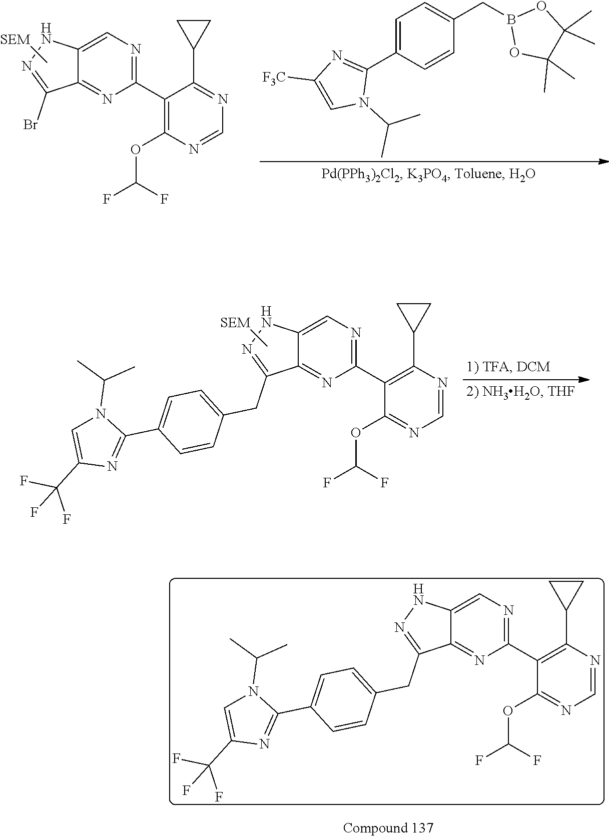

### 1.1 5-(4-cyclopropyl-6-(difluoromethoxy)pyrimidin-5-yl)-3-(4-(1-isopropyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-cyclopropyl-6-(difluoromethoxy)pyrimidin-5-yl)-3-(4-(1-isopropyl-4-(trifluoromethyl)-1H-imidazol-2-yl)benzyl)-2-((2-(trimethylsilyl)ethoxy)methyl)-2H-pyrazolo[4,3-d]pyrimidine

To a solution of 2-[[3-bromo-5-[4-cyclopropyl-6-(difluoromethoxy)pyrimidin-5-yl]pyrazolo[4,3-d]pyrimidin-2-yl]methoxy]ethyl-trimethyl-silane & 2-[[3-bromo-5-[4-cyclopropyl-6-(difluoromethoxy)pyrimidin-5-yl]pyrazolo[4,3-d]pyrimidin-1-yl]methoxy]ethyl-trimethyl-silane (350 mg, 681.72 μmol) in toluene (5 mL) and H₂O (1 mL) were added 1,1′-Bis(diphenylphosphino)ferrocene-palladium(II)dichloride (143 mg, 204 μmol), potassium phosphate tribasic (145 mg, 1.36 mmol) and 1-isopropyl-2-(4-((4,4,5,5-tetramethyl-1,3,2-dioxaborolan-2-yl)methyl)phenyl)-4-(trifluoromethyl)-1H-imidazole (269 mg, 682 μmol) at 25° C. under nitrogen atmosphere. Then the mixture was heated to 90° C., and stirred for 48 hours. The mixture was allowed to cool down to 25° C. The resulting mixture was detected by TLC and LCMS, could find 36% desired product. Then the mixture was diluted with ethyl acetate (100 mL) and was filtered through a Celite pad, the filtrate was concentrated under reduced pressure. The residue was purified by silica gel column chromatography, eluted with 27% ethyl acetate in petroleum ether to give two isomers. The minor isomer fractions were concentrated under reduced pressure to give 5-(4-cyclopropyl-6-(difluoromethoxy)pyrimidin-5-yl)-3-(4-(1-isopropyl-4-(trifluoromethyl)-1H-imidazol-2-yl)benzyl)-1-((2-(trimethylsilyl)ethoxy)methyl)-1H-pyrazolo[4,3-d]pyrimidine (80 mg, crude product) as a brown oil. MS: m/z=701.30 [M+H]⁺. ¹H NMR (400 MHz, Chloroform-d) δ 9.37 (s, 1H), 8.72 (s, 1H), 7.58 (t, J=73.2 Hz, 1H), 7.60-7.56 (m, 2H), 7.48-7.46 (m, 2H), 7.40 (s, 1H), 5.83 (s, 2H), 4.57-4.50 (m, 1H), 3.76 (s, 2H), 3.69-3.65 (m, 2H), 1.44-1.42 (m, 6H), 1.35-1.31 (m, 1H), 1.00-0.99 (m, 2H), 0.98-0.96 (m, 2H), 0.91-0.86 (m, 2H), 0.01 (s, 9H). ¹⁹F NMR (376 MHz, Chloroform-d) δ −62.624, 89.657.

The major isomer fractions were concentrated under reduced pressure to give 5-(4-cyclopropyl-6-(difluoromethoxy)pyrimidin-5-yl)-3-(4-(1-isopropyl-4-(trifluoromethyl)-1H-imidazol-2-yl)benzyl)-2-((2-(trimethylsilyl)ethoxy)methyl)-2H-pyrazolo[4,3-d]pyrimidine (70 mg, crude product) as a brown oil. MS: m/z=701.15 [M+H]⁺. ¹H NMR (400 MHz, Chloroform-d) δ 9.60 (s, 1H), 8.72 (s, 1H), 7.58 (t, J=73.2 Hz, 1H), 7.50-7.48 (m, 4H), 7.41 (s, 1H), 5.78 (s, 2H), 4.56-4.50 (m, 1H), 3.76 (s, 2H), 3.67-3.63 (m, 2H), 1.45-1.43 (m, 6H), 1.37-1.33 (m, 11H), 1.06-1.02 (m, 2H), 0.97-0.93 (m, 2H), 0.90-0.86 (m, 2H), 0.02 (s, 9H). ¹⁹F NMR (376 MHz, Chloroform-d) δ −62.637, 89.582.

### 1.2 5-(4-cyclopropyl-6-(difluoromethoxy)pyrimidin-5-yl)-3-(4-(1-isopropyl-4-(trifluoromethyl)-1H-imidazol-2-yl)benzyl)-1H-pyrazolo[4,3-d]pyrimidine

The solution of 5-(4-cyclopropyl-6-(difluoromethoxy)pyrimidin-5-yl)-3-(4-(1-isopropyl-4-(trifluoromethyl)-1H-imidazol-2-yl)benzyl)-1-((2-(trimethylsilyl)ethoxy)methyl)-1H-pyrazolo[4,3-d]pyrimidine (80 mg, crude product) and 5-(4-cyclopropyl-6-(difluoromethoxy)pyrimidin-5-yl)-3-(4-(1-isopropyl-4-(trifluoromethyl)-1H-imidazol-2-yl)benzyl)-2-((2-(trimethylsilyl)ethoxy)methyl)-2H-pyrazolo[4,3-d]pyrimidine (70 mg, crude product) in dichloromethane (2 mL) and trifluoroacetic acid (2 mL) was stirred for 3 hours at 25° C., the solution was concentrated under reduced pressure. Tetrahydrofuran (2 mL) and ammonium hydroxide (2 mL, 28% aqueous solution) were added to the above residue, the resulting solution was stirred for 10 minutes. The resulting solution was detected by TLC and LCMS, could find 89% desired product. The solution was concentrated under reduced pressure. The residue was purified by Prep-TLC (DCM:MeOH=12:1) to afford crude product, then was further purified by RP-Flash chromatography with the following conditions: Column: C18 spherical, 20-35 um, 100A, 80 g; Mobile Phase A: 10 mM aq. NH₄HCO₃, Mobile Phase B: MeCN; Flow rate: 70 mL/min; Gradient: 5% B to 5% B in 10 min. 5% B to 52% B in 25 min; 52% B to 52% B in 5 min; 52% B to 95% B in 5 min; Detector: UV 254 & 220 nm: RT: 40 min. The collected fractions were combined, concentrated under reduced pressure and lyophilized to give 5-[4-cyclopropyl-6-(difluoromethoxy)pyrimidin-5-yl]-3-[[4-[1-isopropyl-4-(trifluoromethyl)imidazol-2-yl]phenyl]methyl]-1H-pyrazolo[4,3-d]pyrimidine (44.7 mg, 78.35 μmol, 37% yield) as an off-white solid. MS: m/z=571.25 [M+H]⁺. ¹H NMR (400 MHz, Chloroform-d) δ 9.21 (s, 1H), 8.69 (s, 11H), 7.59 (s, 4H), 7.58 (t, J=73.2 Hz, 1H), 7.51 (s, 1H), 4.67-4.60 (m, 1H), 4.51 (s, 2H), 1.87-1.81 (m, 1H), 1.51 (s, 3H), 1.49 (s, 3H), 1.31-1.27 (m, 2H), 0.98-0.94 (m, 2H); ¹⁹F NMR (376 MHz, Chloroform-d) δ −62.02, −89.62.

### Example 92. 5-(4-cyclopropyl-6-methoxy-pyrimidin-5-yl)-3-[[4-[1-(trideuteriomethyl)-4-(trifluoromethyl)imidazol-2-yl]phenyl]methyl]-1H-pyrazolo[4,3-d]pyrimidine Compound 97

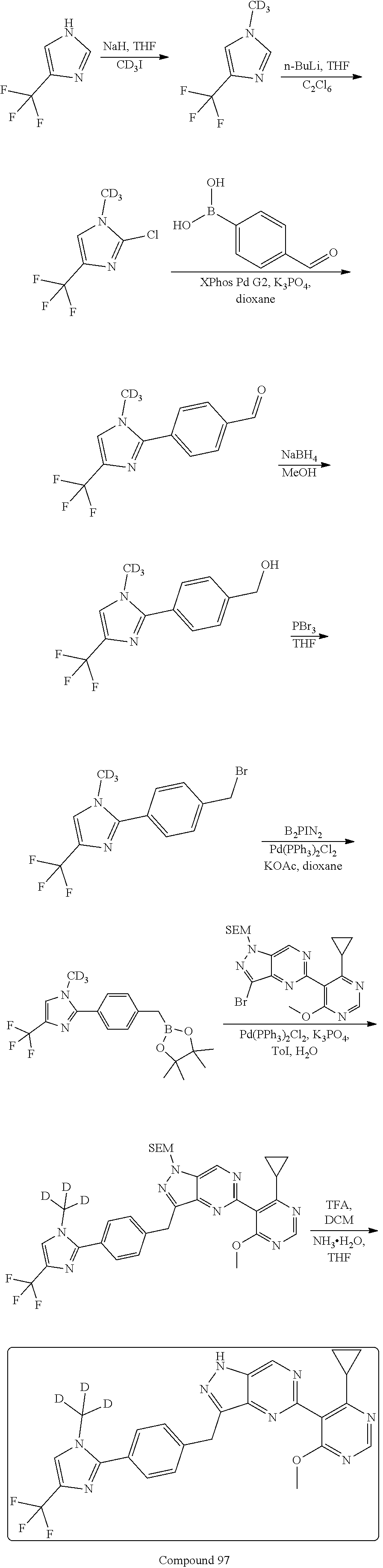

### 1.1 1-(trideuteriomethyl)-4-(trifluoromethyl)imidazole

To a stirred solution of 4-(trifluoromethyl)-1H-imidazole (10.6 g, 77.90 mmol) in tetrahydrofuran (110 mL) was added sodium hydride (60% dispersion in mineral oil, 3.73 g, 93.48 mmol) in portions over 5 min at 0° C. under nitrogen atmosphere. The resulting mixture was stirred for 1 hour at 25° C. under nitrogen atmosphere. To the solution was added trideuterio(iodo)methane (11.3 g, 77.9 mmol, 4.85 mL) dropwise over 5 min at 0° C. under nitrogen atmosphere. The resulting mixture was stirred for 3 hours at 25° C. under nitrogen atmosphere. The reaction was quenched by the addition of saturated ammonium chloride aqueous solution (200 mL) at 0° C. The resulting mixture was extracted with ethyl acetate (3×150 mL). The combined organic layers were washed with brine (50 mL), dried over anhydrous sodium sulfate. After filtration, the filtrate was concentrated under reduced pressure. The residue was purified by silica gel column chromatography, eluted with 1-80% ethyl acetate in petroleum ether to give 1-(trideuteriomethyl)-4-(trifluoromethyl)imidazole (6.7 g, 43.76 mmol, 56% yield) as a yellow oil. MS: m/z=154.05 [M+H]⁺. ¹H NMR (400 MHz, Chloroform-d) δ 7.51 (s, 1H), 7.25 (s, 1H).

### 1.2 2-chloro-1-(trideuteriomethyl)-4-(trifluoromethyl)imidazole

To a stirred solution of 1-(trideuteriomethyl)-4-(trifluoromethyl)imidazole (6.7 g, 43.76 mmol) in tetrahydrofuran (70 mL) was added n-Butyllithium (2.5M in hexane) (17.50 mL, 43.76 mmol) dropwise over 5 min at 0° C. under nitrogen atmosphere. The resulting mixture was stirred for 30 min at 0° C. under nitrogen atmosphere. The resulting mixture was added hexachloroethane (5.18 g, 21.88 mmol) at 0° C. under nitrogen atmosphere. The resulting mixture was stirred for 1 hour at 0° C., and for 3 hours at 25° C. under nitrogen atmosphere. The reaction was quenched by the addition of saturated ammonium chloride aqueous solution (150 mL) at 0° C. The resulting mixture was extracted with ethyl acetate (3×200 mL). The combined organic layers were washed with brine (100 mL), dried over anhydrous sodium sulfate. After filtration, the filtrate was concentrated under reduced pressure. The residue was purified by silica gel column chromatography, eluted with 30%-100% ethyl acetate in petroleum ether to give 2-chloro-1-(trideuteriomethyl)-4-(trifluoromethyl)imidazole (3 g, 15.99 mmol, 36% yield) as a yellow oil. MS: m/z=188.15 [M+H]⁺. ¹H NMR (400 MHz, Chloroform-d) δ 7.26 (s, 1H).

### 1.3 4-[1-(trideuteriomethyl)-4-(trifluoromethyl)imidazol-2-yl]benzaldehyde

To a stirred solution of 2-chloro-1-(trideuteriomethyl)-4-(trifluoromethyl)imidazole (1.5 g, 8.00 mmol) and (4-formylphenyl)boronic acid (1.80 g, 12.00 mmol) in 1,4-dioxane (15 mL) were added potassium phosphate (5.09 g, 23.99 mmol) and chloro(2-dicyclohexylphosphino-2′,4′,6′-tri-i-propyl-1,1′-biphenyl)(2′-amino-1,1′-biphenyl-2-yl) palladium(1) (314 mg, 399.9 μmol) at 25° C. under nitrogen atmosphere. The resulting mixture was stirred for 16 hours at 90° C. under nitrogen atmosphere. The reaction was monitored by LCMS. The resulting mixture was filtered, the filter cake was washed with ethyl acetate (3×5 mL). The filtrate was concentrated under reduced pressure. The residue was purified by silica gel column chromatography, eluted with 1-50% ethyl acetate in petroleum ether to give 4-[1-(trideuteriomethyl)-4-(trifluoromethyl)imidazol-2-yl]benzaldehyde (1.25 g, 4.86 mmol, 60% yield) as a yellow solid. MS: m/z=258.00 [M+H]⁺. ¹H NMR (400 MHz, Chloroform-d) δ 10.11 (s, 1H), 8.06-7.99 (m, 2H), 7.92-7.85 (m, 2H), 7.40 (s, 1H).

### 1.4 [4-[1-(trideuteriomethyl)-4-(trifluoromethyl)imidazol-2-yl]phenyl]methanol

To a stirred solution of 4-[1-(trideuteriomethyl)-4-(trifluoromethyl)imidazol-2-yl]benzaldehyde (400 mg, 1.56 mmol) in methanol (4 mL) was added sodium boranuide (117.66 mg, 3.11 mmol,) at 25° C. under nitrogen atmosphere. The resulting mixture was stirred for 2 hours at 25° C. under nitrogen atmosphere. The reaction was monitored by LCMS. The reaction was quenched by the addition of water (0.1 mL) at 0° C. The resulting mixture was concentrated under reduced pressure. The residue was purified by silica gel column chromatography, eluted with 1-50% ethyl acetate in petroleum ether to give [4-[1-(trideuteriomethyl)-4-(trifluoromethyl)imidazol-2-yl]phenyl]methanol (360 mg, 1.39 mmol, 89% yield) as a white solid. MS: m/z=260.05 [M+H]⁺. ¹H NMR (400 MHz, Chloroform-d) δ 7.65 (d, J=7.6 Hz, 2H), 7.49 (d, J=8.0 Hz, 2H), 7.34 (s, 1H), 4.80 (s, 2H).

### 1.5 2-[4-(bromomethyl)phenyl]-1-(trideuteriomethyl)-4-(trifluoromethyl)imidazole

To a stirred solution of [4-[1-(trideuteriomethyl)-4-(trifluoromethyl)imidazol-2-yl]phenyl]methanol (300 mg, 1.16 mmol) in tetrahydrofuran (3 mL) was added tribromophosphane (939.8 mg, 3.47 mmol) dropwise at 0° C. under nitrogen atmosphere. The resulting mixture was stirred for 2 hours at 25° C. under nitrogen atmosphere. The reaction was monitored by LCMS. The reaction was quenched by the addition of saturated sodium bicarbonate aqueous solution (5 mL) at 0° C. The resulting mixture was extracted with ethyl acetate (3×15 mL). The combined organic layers were washed with brine (10 mL), dried over anhydrous sodium sulfate. After filtration, the filtrate was concentrated under reduced pressure. The residue was purified by silica gel column chromatography, eluted with 1-30% ethyl acetate in petroleum ether to give 2-[4-(bromomethyl)phenyl]-1-(trideuteriomethyl)-4-(trifluoromethyl)imidazole (300 mg, 0.93 mmol, 80% yield) as an off-white solid. MS: m/z=321.95, 323.95 [M+H]⁺. ¹H NMR (400 MHz, Chloroform-d) δ 7.69-7.62 (m, 2H), 7.56-7.44 (m, 2H), 7.34 (s, 1H), 4.55 (s, 2H).

### 1.6 2-[4-[(4,4,5,5-tetramethyl-1,3,2-dioxaborolan-2-yl)methyl]phenyl]-1-(trideuteriomethyl)-4-(trifluoromethyl)imidazole

To a stirred solution of 2-[4-(bromomethyl)phenyl]-1-(trideuteriomethyl)-4-(trifluoromethyl)imidazole (300 mg, 0.93 mmol) and 4,4,5,5-tetramethyl-2-(4,4,5,5-tetramethyl-1,3,2-dioxaborolan-2-yl)-1,3,2-dioxaborolane (355 mg, 1.40 mmol) in 1,4-dioxane (5 mL) were added bis(triphenylphosphine)palladium(IT) chloride (654 mg, 0.93 mmol) and potassium phosphate (394 mg, 1.86 mmol) at 25° C. under nitrogen atmosphere. The resulting mixture was stirred for 16 hours at 80° C. under nitrogen atmosphere. The reaction was monitored by LCMS. The resulted mixture were filtered, the filter cake was washed with ethyl acetate (3×3 mL). The filtrate was concentrated under reduced pressure. The resulting mixture was diluted with water (20 mL). The resulting mixture was extracted with ethyl acetate (3×40 mL). The combined organic layers were washed with brine (20 mL), dried over anhydrous sodium sulfate. After filtration, the filtrate was concentrated under reduced pressure. The residue was purified by silica gel column chromatography, eluted with 1-30% ethyl acetate in petroleum ether to give 2-[4-[(4,4,5,5-tetramethyl-1,3,2-dioxaborolan-2-yl)methyl]phenyl]-1-(trideuteriomethyl)-4-(trifluoromethyl)imidazole (255 mg, 689.17 μmol, 74% yield) as a white solid. MS: m/z=370.10 [M+H]⁺.

### 1.7 2-[[5-[4-cyclopropyl-6-(trideuteriomethoxy)pyrimidin-5-yl]-3-[[4-[1-methyl-4-(trifluoromethyl)imidazol-2-yl]phenyl]methyl]pyrazolo[4,3-d]pyrimidin-1-yl]methoxy]ethyl-trimethyl-silane

To a stirred solution of 2-[[3-bromo-5-[4-cyclopropyl-6-(trideuteriomethoxy)pyrimidin-5-yl]pyrazolo[4,3-d]pyrimidin-1-yl]methoxy]ethyl-trimethyl-silane (255 mg, 530.75 μmol) and 1-methyl-2-[4-[(4,4,5,5-tetramethyl-1,3,2-dioxaborolan-2-yl)methyl]phenyl]-4-(trifluoromethyl)imidazole (213.79 mg, 583.83 μmol) in water (1 mL) and toluene (5 mL) were added bis(triphenylphosphine)palladium(II) chloride (76 mg, 106.15 μmol) and potassium phosphate (337 mg, 1.59 mmol) at 25° C. under nitrogen atmosphere. The resulting mixture was stirred for 16 hours at 90° C. under nitrogen atmosphere. The reaction was monitored by LCMS. The resulting mixture were filtered, the filter cake was washed with ethyl acetate (3×3 mL). The filtrate was concentrated under reduced pressure. The residue was purified by silica gel column chromatography, eluted with 1-80% ethyl acetate in petroleum ether to give 2-[[5-[4-cyclopropyl-6-(trideuteriomethoxy)pyrimidin-5-yl]-3-[[4-[1-methyl-4-(trifluoromethyl)imidazol-2-yl]phenyl]methyl]pyrazolo[4,3-d]pyrimidin-1-yl]methoxy]ethyl-trimethyl-silane (140 mg, 218.83 μmol, 41% yield) as a yellow solid. MS: m/z=640.20 [M+H]⁺.

### 1.8 5-(4-cyclopropyl-6-methoxy-pyrimidin-5-yl)-3-[[4-[1-(trideuteriomethyl)-4-(trifluoromethyl)imidazol-2-yl]phenyl]methyl]-1H-pyrazolo[4,3-d]pyrimidine

To a stirred solution of 2-[[5-(4-cyclopropyl-6-methoxy-pyrimidin-5-yk)-3-[[4-[1-(trideuteriomethyl)-4-(trifluoromethyl)imidazol-2-yl]phenyl]methyl]pyrazolo[4,3-d]pyrimidin-1-yl]methoxy]ethyl-trimethyl-silane (170 mg, 265.72 μmol) in dichloromethane (1 mL) was added 2,2,2-trifluoroacetic acid (1.48 g, 12.98 mmol, 1 mL) at 25° C. under nitrogen atmosphere and then stirred at this temperature 1 hour. The resulting mixture was concentrated under reduced pressure. To a stirred solution of the residue in tetrahydrofuran (1 mL) was added ammonium hydroxide (28% aqueous solution, 1 mL) at 25° C. under nitrogen atmosphere and then stirred at this temperature 1 hour. The resulting mixture was concentrated under reduced pressure. The obtained product was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 95% B in 20 min; Detector: UV 254 & 210 nm; RT: 18 min. The collected fractions were combined, concentrated under reduced pressure and lyophilized to give 5-(4-cyclopropyl-6-methoxy-pyrimidin-5-yl)-3-[[4-[1-(trideuteriomethyl)-4-(trifluoromethyl)imidazol-2-yl]phenyl]methyl]-1H-pyrazolo[4,3-d]pyrimidine (66 mg, 129.54 μmol, 48% yield) as a white solid. MS: m/z=510.20 [M+H]⁺. ¹H NMR (400 MHz, Chloroform-d) δ 13.55 (s, 1H), 9.17 (s, 1H), 8.69 (s, 1H), 7.69 (d, J=8.4 Hz, 2H), 7.60 (d, J=8.0 Hz, 2H), 7.41 (s, 1H), 4.55 (s, 2H), 3.94 (s, 3H), 1.70-1.61 (m, 1H), 1.25-1.22 (m, 2H), 0.85-0.81 (m, 2H). ¹⁹F NMR (376 MHz, Chloroform-d) δ −62.16.

### Example 93. 5-[4-(1-fluorocyclopropyl)-6-methoxy-pyrimidin-5-yl]-3-[[4-[1-methyl-4-(trifluoromethyl)imidazol-2-yl]phenyl]methyl]-1H-pyrazolo[4,3-d]pyrimidine Compound 202

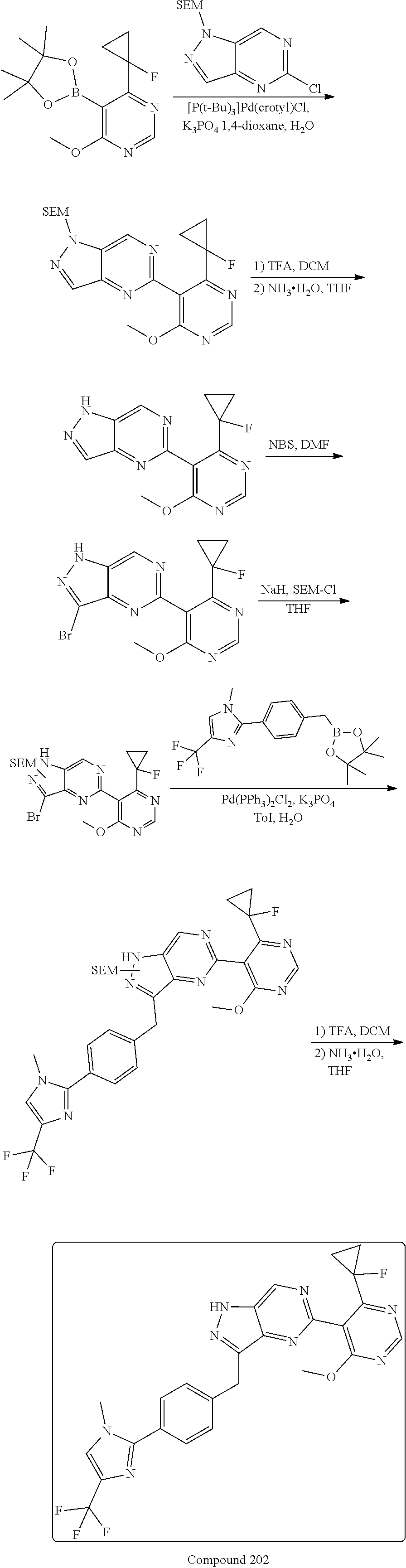

### 1.1 2-[[5-[4-(1-fluorocyclopropyl)-6-methoxy-pyrimidin-5-yl]pyrazolo[4,3-d]pyrimidin-1-yl]methoxy]ethyl-trimethyl-silane

To a solution of 4-(1-fluorocyclopropyl)-6-methoxy-5-(4,4,5,5-tetramethyl-1,3,2-dioxaborolan-2-yl)pyrimidine (700 mg, 2.38 mmol) in 1,4-dioxane (30 mL) and water (5 mL) was added potassium phosphate (1.01 g, 4.76 mmol) and chloro(crotyl)(tri-tert-butylphosphine)palladium(II) (190 mg, 476 μmol) at 25° C. The above mixture was stirred at 90° C. for 3 hours under nitrogen atmosphere. The resulted solution was cooled down to room temperature, quenched with brine (50 mL), extracted with ethyl acetate (3×100 mL). The combined organic layers were dried over anhydrous sodium sulfate. After filtration, the filtrate was concentrated under reduced pressure. The residue was purified by silica gel column chromatography, eluted with 1%-7% methanol in dichloromethane to give 2-[[5-[4-(1-fluorocyclopropyl)-6-methoxy-pyrimidin-5-yl]pyrazolo[4,3-d]pyrimidin-1-yl]methoxy]ethyl-trimethyl-silane (800 mg, 1.92 mmol, 80% yield) as a yellow oil. MS-m/z=417.15 [M+H]⁺.

### 1.2 5-[4-(1-fluorocyclopropyl)-6-methoxy-pyrimidin-5-yl]-1H-pyrazolo[4,3-d]pyrimidine

To a solution of 2-[[5-[4-(1-fluorocyclopropyl)-6-methoxy-pyrimidin-5-yl]pyrazolo[4,3-d]pyrimidin-1-yl]methoxy]ethyl-trimethyl-silane (700 mg, 1.68 mmol) in dichloromethane (7 mL) was added trifluoroacetic acid (7 mL) at 0° C. under nitrogen atmosphere. The reaction mixture was stirred at 25° C. for 1 hour. The reaction mixture was concentrated under reduced pressure. The residue was dissolved in tetrahydrofuran (7 mL), then ammonium hydroxide (7 mL) was added at 0° C. The resulted mixture stirred at 25° C. for 1 hour and then concentrated under reduced pressure. The residue was purified by silica gel column chromatography, eluted with 1%-10% methanol in dichloromethane to give 5-[4-(1-fluorocyclopropyl)-6-methoxy-pyrimidin-5-yl]-1H-pyrazolo[4,3-d]pyrimidine (310 mg, 1.08 mmol, 64% yield) as an off-white solid. MS: m/z=287.15 [M+H]⁺. ¹H NMR (400 MHz, Chloroform-d) δ 9.39 (s, 1H), 8.72 (s, 1H), 8.43 (s, 1H), 3.95 (s, 3H), 1.62-1.58 (m, 2H), 1.42-1.26 (m, 2H). ¹⁹F NMR (376 MHz, Chloroform-d) δ −188.95.

### 1.3 3-bromo-5-[4-(1-fluorocyclopropyl)-6-methoxy-pyrimidin-5-yl]-1H-pyrazolo[4,3-d]p yrimidine

To a solution of 5-[4-(1-fluorocyclopropyl)-6-methoxy-pyrimidin-5-yl]-1H-pyrazolo[4,3-d]pyrimidine (310 mg, 1.08 mmol) in N,N-dimethyl formamide (6 mL) was added 1-bromopyrrolidine-2,5-dione (289 mg, 1.62 mmol) at 25° C. under nitrogen atmosphere. The reaction mixture was stirred at 25° C. for 16 hours. The reaction mixture was concentrated under reduced pressure. The residue was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 50 mL/min Gradient: 5% B to 5% B in 2 min, 5% B to 31% B in 19 min, 31% to 31% B in 2 min, 31% B to 95% B in 20 min; Detector: UV 254 & 210 nm; RT: 22 min. The collected fractions were combined, concentrated and then lyophilized overnight to give 3-bromo-5-[4-(1-fluorocyclopropyl)-6-methoxy-pyrimidin-5-yl]-1H-pyrazolo[4,3-d]pyrimidine (314 mg, 859.90 μmol, 79% yield) as a light-yellow solid. MS: m/z=365.15, 367.15 [M+H]⁺. ¹H NMR (400 MHz, Chloroform-d) δ 9.36 (s, 1H), 8.72 (s, 1H), 3.95 (s, 3H), 1.65-1.55 (m, 2H), 1.43-1.26 (m, 2H). ¹⁹F NMR (376 MHz, Chloroform-d) δ −188.59.

### 1.4 3-bromo-5-(4-(1-fluorocyclopropyl)-6-methoxypyrimidin-5-yl)-1-((2-(trimethylsilyl)ethoxy)methyl)-1H-pyrazolo[4,3-d]pyrimidine & 3-bromo-5-(4-(1-fluorocyclopropyl)-6-methoxypyrimidin-5-yl)-2-((2-(trimethylsilyl)ethoxy)methyl)-2H-pyrazolo[4,3-d]pyrimidine

To a solution of 3-bromo-5-[4-(1-fluorocyclopropyl)-6-methoxy-pyrimidin-5-yl]-1H-pyrazolo[4,3-d]pyrimidine (314 mg, 859.90 μmol) in tetrahydrofuran (5 mL) was added sodium hydride (38 mg, 946 μmol, 60% dispersion in oil) in portions at 0° C. under nitrogen atmosphere. The reaction mixture was stirred at 0° C. for 45 minutes. Then 2-(chloromethoxy)ethyl-trimethyl-silane (158 mg, 946 μmol, 167 μL) was added to the reaction mixture at 0° C. The reaction mixture stirred at 25° C. for 2 hours. The reaction was quenched with saturated ammonium chloride aqueous solution (50 mL), then extracted with ethyl acetate (3×30 mL). The combined organic extracts were washed with brine (20 mL), dried over anhydrous sodium sulfate. After filtration, the filtrate was concentrated under reduced pressure. The residue was purified by silica gel column chromatography, eluted with 1%-8% methanol in dichloromethane ether to give 3-bromo-5-(4-(1-fluorocyclopropyl)-6-methoxypyrimidin-5-yl)-1-((2-(trimethylsilyl)ethoxy)methyl)-1H-pyrazolo[4,3-d]pyrimidine & 3-bromo-5-(4-(1-fluorocyclopropyl)-6-methoxypyrimidin-5-yl)-2-((2-(trimethylsilyl)ethoxy)methyl)-2H-pyrazolo[4,3-d]pyrimidine (mixture of two isomers, 330 mg, 666.10 μmol, 77% yield) as a light-yellow solid. MS: m/z=495.10, 497.10 [M+H]⁺.

### 1.5 5-(4-(1-fluorocyclopropyl)-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-(1-fluorocyclopropyl)-6-methoxypyrimidin-5-yl)-3-(4-(1-methyl-4-(trifluoromethyl)-1H-imidazol-2-yl)benzyl)-2-((2-trimethylsilyl)ethoxy)methyl)-2H-pyrazolo[4,3-d]pyrimidine

To a solution of 3-bromo-5-(4-(1-fluorocyclopropyl)-6-methoxypyrimidin-5-yl)-1-((2-(trimethylsilyl)ethoxy)methyl)-1H-pyrazolo[4,3-d]pyrimidine & 3-bromo-5-(4-(1-fluorocyclopropyl)-6-methoxypyrimidin-5-yl)-2-((2-(trimethylsilyl)ethoxy)methyl)-2H-pyrazolo[4,3-d]pyrimidine (mixture of two isomers, 285 mg, 0.575 mmol) and 1-methyl-2-[4-[(4,4,5,5-tetramethyl-1,3,2-dioxaborolan-2-yl)methyl]phenyl]-4-(trifluoromethyl)imidazole (316 mg, 0.863 mmol) in toluene (5 mL) and water (1 mL) were added dichloropalladium triphenylphosphane (121 mg, 0.173 mmol) and potassium phosphate (244 mg, 1.15 mmol) at 25° C. under nitrogen atmosphere. The reaction mixture was stirred at 100° C. for 16 hours. The mixture was cooled down to 25° C. The solid was filtered out. The filtrate was concentrated under reduced pressure. The residue was purified by silica gel column chromatography, eluted with 40%-70% ethyl acetate in petroleum ether to give 5-(4-(1-fluorocyclopropyl)-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-(1-fluorocyclopropyl)-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (mixture of two isomers, 150 mg, 229.10 μmol, 39% yield) as an off-white solid. MS: m/z=655.25, 657.25 [M+H]⁺.

### 1.6 5-[4-(1-fluorocyclopropyl)-6-methoxy-pyrimidin-5-yl]-3-[[4-[1-methyl-4-(trifluoromethyl)imidazol-2-yl]phenyl]methyl]-1H-pyrazolo[4,3-d]pyrimidine

To a solution of 5-(4-(1-fluorocyclopropyl)-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-(1-fluorocyclopropyl)-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (mixture of two isomers, 150 mg, 229.10 μmol) in dichloromethane (2 mL) was added trifluoroacetic acid (2 mL) at 0° C. under nitrogen atmosphere. The reaction mixture was stirred at 25° C. for 2 hours. The reaction solution was concentrated under reduced pressure. Then the residue was dissolved in tetrahydrofuran (2 mL) and ammonium hydroxide (2 mL) then stirred at 25° C. for 1 hour. The resulted mixture was concentrated under reduced pressure. The residue was purified by silica gel column chromatography, eluted with 60%-100% ethyl acetate in petroleum ether to give a crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 25 mL/min; Gradient: 5% B to 5% B in 3 min, 5% B to 43% B in 20 min; 43% B to 43% B in 3 min, 43% B to 95% B in 15 min; Detector: UV 254 & 210 nm; RT: 24 min. The collected fractions were combined, concentrated and then lyophilized overnight to give 5-[4-(1-fluorocyclopropyl)-6-methoxy-pyrimidin-5-yl]-3-[[4-[1-methyl-4-(trifluoromethyl)imidazol-2-yl]phenyl]methyl]-1H-pyrazolo[4,3-d]pyrimidine (45 mg, 85.80 μmol, 37% yield) as an off-white solid. MS: m/z=525.35 [M+H]⁺. ¹H NMR (400 MHz, Chloroform-d) 9.21 (s, 1H), 8.71 (s, 1H), 7.61 (d, J=8.4 Hz, 2H), 7.52 (d, J=8.4 Hz, 2H), 7.38 (s, 1H), 4.54 (s, 2H), 3.94 (s, 3H), 3.79 (s, 3H), 1.59-1.51 (m, 2H), 1.33-1.19 (m, 2H). ¹⁹F NMR (376 MHz, Chloroform-d) δ −62.30, −188.12.

### Example 94. 2-[4-cyclopropyl-6-(difluoromethoxy)pyrimidin-5-yl]-7-[[4-[1-isopropyl-4-(trifluoromethyl)imidazol-2-yl]phenyl]methyl]-5H-pyrrolo[3,2-d]pyrimidine Compound 92 and Compound 132

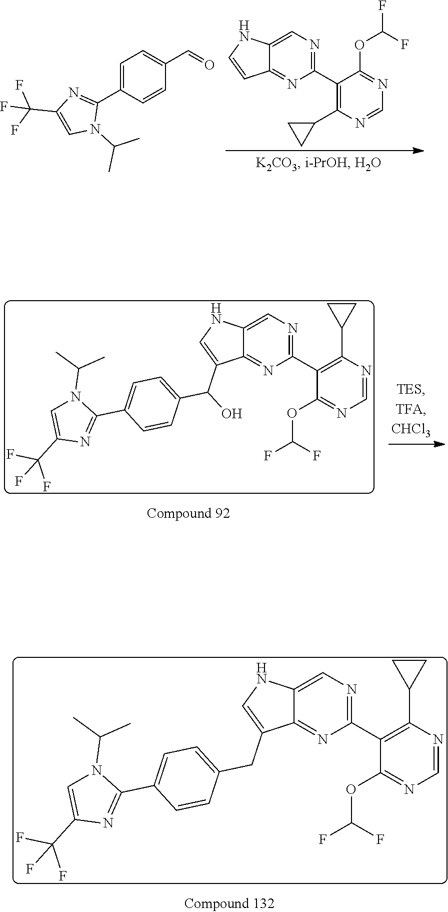

### 1.1 [2-[4-cyclopropyl-6-(difluoromethoxy)pyrimidin-5-yl]-5H-pyrrolo[3,2-d]pyrimidin-7-yl]-[4-[1-isopropyl-4-(trifluoromethyl)imidazol-2-yl]phenyl]methanol

To a stirring solution of 2-[4-cyclopropyl-6-(difluoromethoxy)pyrimidin-5-yl]-5H-pyrrolo[3,2-d]pyrimidine (150 mg, 494.61 μmol) in isopropyl alcohol (2.00 mL) was added 4-[1-isopropyl-4-(trifluoromethyl)imidazol-2-yl]benzaldehyde (140 mg, 495 μmol) at 0° C. The resulting mixture was stirred for 10 min at 0° C. To the above mixture was added potassium carbonate (82 mg, 593 μmol) in water (2 mL) at 0° C. The resulting mixture was stirred under nitrogen atmosphere at 60° C. for 16 hours. The reaction mixture was cooled down to room temperature. The reaction was monitored by LCMS. The reaction mixture was diluted with water (10 mL). The resulted mixture was extracted with ethyl acetate (3×10 mL). The combined organic layers were washed with brine (3×10 mL), dried over anhydrous sodium sulfate. After filtration, the filtrate was concentrated under reduced pressure. The residue was purified by silica gel column chromatography, eluted with 3% methanol in dichloromethane to afford 80 mg crude product. The crude product was further purified by reverse phase chromatography with the following conditions: Column: C18 spherical Column, 20-35 um, 100A, 20 g; Mobile Phase A: Water (5 mM aq. NH₄HCO₃), Mobile Phase B: MeCN; Flow rate: 35 mL/min; Gradient: 5% B to 5% B in 5 min, 5% B to 38% B in 10 min, 38% B to 38% B in 2 min, 38% B to 95% B in 10 min; Detector; UV 254 & 210 nm; RT: 16 min. The product-containing fractions were collected and evaporated in vacuo and then lyophilized overnight to give [2-[4-cyclopropyl-6-(difluoromethoxy)pyrimidin-5-yl]-5H-pyrrolo[3,2-d]pyrimidin-7-yl]-[4-[1-isopropyl-4-(trifluoromethyl)imidazol-2-yl]phenyl]methanol (50 mg, 85.39 μmol, 17% yield) as an off-white solid. MS: m/z=586.25 [M+H]⁺. ¹H NMR (400 MHz, DMSO-d₆) δ 11.95 (s, 1H), 9.05 (s, 1H), 8.80 (s, 1H), 8.15 (s, 1H), 7.87 (t, J=2.4 Hz, 1H), 7.82 (t, J=71.6 Hz, 1H), 7.68 (d, J=8.0 Hz, 2H), 7.47 (d, J=8.0 Hz, 2H), 6.21 (d, J=4.4 Hz, 11H), 5.95 (d, J=4.4 Hz, 1H), 4.48-4.41 (m, 1H), 1.81-1.75 (m, 1H), 1.38 (d, J=6.8 Hz, 6H), 1.14-1.10 (m, 2H), 0.97-0.90 (m, 2H). ¹⁹F NMR (282 MHz, DMSO-d₆) δ −60.715, −88.475, −88.506.

### 1.2 2-[4-cyclopropyl-6-(difluoromethoxy)pyrimidin-5-yl]-7-[[4-[1-isopropyl-4-(trifluoromethyl)imidazol-2-yl]phenyl]methyl]-5H-pyrrolo[3,2-d]pyrimidine

To a stirred solution of [2-[4-cyclopropyl-6-(difluoromethoxy)pyrimidin-5-yl]-5H-pyrrolo[3,2-d]pyrimidin-7-yl]-[4-[1-isopropyl-4-(trifluoromethyl)imidazol-2-yl]phenyl]methanol (45 mg, 77 μmol) in chloroform (2 mL) were added trifluoroacetic acid (1 mL) and triethoxysilane (2 mL) under nitrogen atmosphere at 25° C. The resulting mixture was stirred at 25° C. for 2 hours. The reaction was monitored by LCMS. The reaction solution was concentrated under reduced pressure. The residue was purified by reverse phase chromatography with the following conditions: C18 spherical Column, 20-35 um, 100A, 40 g; Mobile Phase A: Water (5 mM aq. NH₄HCO₃), Mobile Phase B: MeCN; Flow rate: 25 mL/min; Gradient: 5% B to 5% B in 5 min, 5% B to 48% B in 13 min, 48% B to 48% B in 2 min, 48% B to 95% B in 5 min; Detector: UV 254 & 210 nm; RT: 20 min. The product-containing fractions were collected and evaporated in vacuo and then lyophilized overnight to give 2-[4-cyclopropyl-6-(difluoromethoxy)pyrimidin-5-yl]-7-[[4-[1-isopropyl-4-(trifluoromethyl)imidazol-2-yl]phenyl]methyl]-5H-pyrrolo[3,2-d]pyrimidine (21.8 mg, 38.28 μmol, 50% yield) as an off-withe solid. MS: m/z=570.30 [M+H]⁺. ¹H NMR (400 MHz, Chloroform-d) δ 11.72 (br., 1H), 9.24 (s, 1H), 8.72 (s, 1H), 7.68-7.62 (m, 1H), 7.54-7.45 (m, 5H), 7.33-7.28 (m, 1H), 4.56-4.47 (m, 1H), 4.31 (s, 2H), 1.97-1.86 (m, 1H), 1.46 (d, J=6.4 Hz, 6H), 1.39-1.33 (m, 2H), 1.11-0.92 (m, 2H). ¹⁹F NMR (282 MHz, Chloroform-d) δ −62.451, −89.294.

### Example 95. 2-(4-cyclopropyl-6-methoxy-pyrimidin-5-yl)-7-[[3,5-difluoro-4-[1-methyl-4-(trifluoromethyl)imidazol-2-yl]phenyl]methyl]-5H-pyrrolo[3,2-d]pyrimidine Compound 117

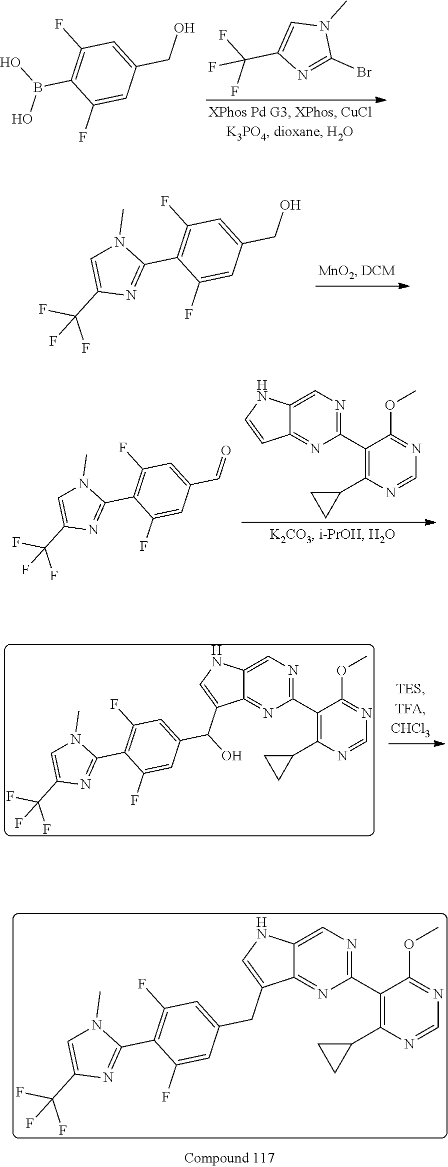

### 1.1 [3,5-difluoro-4-[1-methyl-4-(trifluoromethyl)imidazol-2-yl]phenyl]methanol

To a solution of [2,6-difluoro-4-(hydroxymethyl)phenyl]boronic acid (430 mg, 2.29 mmol), 2-bromo-1-methyl-4-(trifluoromethyl)imidazole (524 mg, 2.29 mmol) and cuprous chloride (227 mg, 2.29 mmol) in 1,4-dioxane (10 mL) and water (2 mL) were added methanesulfonato(2-dicyclohexylphosphino-2′,4′,6′-tri-i-propyl-1,1′-biphenyl)(2′-amino-1,1′-biphenyl-2-yl)palladium(1) (194 mg, 0.229 mmol), dicyclohexyl-[2-(2,4,6-triisopropylphenyl)phenyl]phosphane (109 mg, 0.229 mmol) and potassium phosphate (971 mg, 4.58 mmol) at 25° C. under nitrogen atmosphere and then stirred at 90° C. under nitrogen atmosphere for 16 hours. The resulted mixture was cooled down to room temperature and quenched by 5% ammonium hydroxide aqueous solution (50 mL), extracted with ethyl acetate (3×40 mL). The organic layers were combined, washed with brine (3×50 mL), dried over anhydrous sodium sulfate. After filtration, the filtrate was concentrated under reduced pressure. The residue was purified by silica gel column chromatography, eluted with 1%-67% ethyl acetate in petroleum ether to give [3,5-difluoro-4-[1-methyl-4-(trifluoromethyl)imidazol-2-yl]phenyl]methanol (182 mg, 622.85 μmol, 27% yield) as a yellow oil. MS: m/z=293.00 [M+H]⁺. ¹H NMR (400 MHz, Chloroform-d) δ 7.43 (s, 1H), 7.02-7.69 (m, 2H), 4.76 (s, 2H), 3.61 (s, 3H). ¹⁹F NMR (377 MHz, Chloroform-d) δ −62.66, −109.87.

### 1.2 3,5-difluoro-4-[1-methyl-4-(trifluoromethyl)imidazol-2-yl]benzaldehyde

To a solution of [3,5-difluoro-4-[1-methyl-4-(trifluoromethyl)imidazol-2-yl]phenyl]methanol (180 mg, 616.01 μmol) in dichloromethane (10 mL) was added dioxomanganese (1.34 g, 12.32 mmol, 80% purity) at 25° C., and then stirred at 40° C. for 16 hours. The resulting mixture was cooled down to room temperature and filtered. The filtrate was concentrated under reduced pressure. The residue was purified by silica gel column chromatography, eluted with 1%-50% ethyl acetate in petroleum ether to afford 3,5-difluoro-4-[1-methyl-4-(trifluoromethyl)imidazol-2-yl]benzaldehyde (41.3 mg, 142.32 μmol, 23% yield) as a yellow oil. MS: m/z=290.95. ¹H NMR (400 MHz, Chloroform-d) δ 10.14 (s, 1H), 7.62-7.54 (m, 1H), 7.17-7.06 (m, 2H), 3.89 (s, 3H). ¹⁹F NMR (376 MHz, Chloroform-d) δ −59.33, −109.35.

### 1.3 [2-(4-cyclopropyl-6-methoxy-pyrimidin-5-yl)-5H-pyrrolo[3,2-d]pyrimidin-7-yl]-[3,5-difluoro-4-[1-methyl-4-(trifluoromethyl)imidazol-2-yl]phenyl]methanol

To a solution of 3,5-difluoro-4-[1-methyl-4-(trifluoromethyl)imidazol-2-yl]benzaldehyde (41.3 mg, 142.32 μmol) and 2-(4-cyclopropyl-6-methoxy-pyrimidin-5-yl)-5H-pyrrolo[3,2-d]pyrimidine (38 mg, 142 μmol) in isopropanol (0.8 mL) under nitrogen atmosphere were added potassium carbonate (20 mg, 142 μmol) and water (0.8 mL) at 25° C., and then stirred at 40° C. for 16 hours. The resulted mixture were cooled down to room temperature and filtered, the filtrate was concentrated under reduced pressure. The residue was purified by RP-Flash with the following conditions: Column: C18 spherical, 20-30 um, 100A, 40 g; Mobile Phase A: Water (10 mM aq. NH₄HCO₃), Mobile Phase B: MeCN; Flow rate: 35 mL/min; Gradient: 2% B to 2% B in 5 min, 2% B to 54% B in 23 min; 54% B to 54% B in 2.4 min; 54% B to 98% B in 5 min; 98% B to 98% B in 5 min; Detector: UV 254 & 210 nm; RT: 29 min. The collected fractions were combined, concentrated and then lyophilized to give [2-(4-cyclopropyl-6-methoxy-pyrimidin-5-yl)-5H-pyrrolo[3,2-d]pyrimidin-7-yl]-[3,5-difluoro-4-[1-methyl-4-(trifluoromethyl)imidazol-2-yl]phenyl]methanol (28.4 mg, 50.94 μmol, 35% yield) as an off-white solid. MS: m/z=558.25 [M+H]⁺. ¹H NMR (400 MHz, Chloroform-d) δ 10.98 (s, 1H), 8.97 (s, 1H), 8.65 (s, 1H), 7.48 (s, 1H), 7.33-7.28 (m, 3H), 6.34 (s, 1H), 4.70 (br., 1H), 3.91 (s, 3H), 3.66 (s, 3H), 1.75-1.69 (m, 1H), 1.28-1.14 (m, 2H), 0.94-0.84 (m, 2H). ¹⁹F NMR (377 MHz, Chloroform-d) δ −62.40, −109.73.

### 1.4 2-(4-cyclopropyl-6-methoxy-pyrimidin-5-yl)-7-[[3,5-difluoro-4-[1-methyl-4-(trifluoromethyl)imidazol-2-yl]phenyl]methyl]-5H-pyrrolo[3,2-d]pyrimidine

To a solution of [2-(4-cyclopropyl-6-methoxy-pyrimidin-5-yl)-5H-pyrrolo[3,2-d]pyrimidin-7-yl]-[3,5-difluoro-4-[1-methy-4-(trifluoromethyl)imidazol-2-yl]phenyl]methanol (25 mg, 45 μmol) and triethylsilane (1 mL) in chloroform (1 mL) was added 2,2,2-trifluoroacetic acid (0.5 mL) at 0° C., and then stirred at 25° C. for 36 hours. The resulting mixture was concentrated under reduced pressure. The residue was purified by Prep-TLC, eluted with 9% methanol in dichloromethane to give a crude product. The crude product was purified by RP-Flash chromatography with the following conditions: Column: C18 spherical, 20-30 um, 100A, 40 g; Mobile Phase A: Water (10 mM aq. NH₄HCO₃), Mobile Phase B: MeCN; Flow rate: 35 mL/min; Gradient: 2% B to 2% B in 5 min, 2% B to 57% B in 25 min; 57% B to 57% B in 3.6 min; 57% B to 98% B in 5 min; 98% B to 98% B in 5 min; Detector: UV 254 & 210 nm; RT: 32 min. The collected fractions were combined, concentrated and then lyophilized to give 2-(4-cyclopropyl-6-methoxy-pyrimidin-5-yl)-7-[[3,5-difluoro-4-[1-methyl-4-(trifluoromethyl)imidazol-2-yl]phenyl]methyl]-5H-pyrrolo[3,2-d]pyrimidine (5.5 mg, 10.16 μmol, 22% yield) as an off-white solid. MS: m/z=542.40 [M+H]⁺. ¹H NMR (400 MHz, Chloroform-d) δ 10.89 (s, 1H), 8.99 (s, 1H), 8.66 (s, 1H), 7.48 (s, 1H), 7.35 (s, 1H), 7.05 (d, J=8.4 Hz, 2H), 4.27 (s, 2H), 3.92 (s, 3H), 3.66 (s, 3H), 1.74-1.68 (m, 1H), 1.28-1.21 (m, 2H), 0.89-0.85 (m, 2H). ¹⁹F NMR (376 MHz, Chloroform-d) δ −62.36, −110.78.

### Example 96. 5-[4-(2,2-difluorocyclopropyl)-6-methoxy-pyrimidin-5-yl]-3-[[4-[1-methyl-4-(trifluoromethyl)imidazol-2-yl]phenyl]methyl]-1H-pyrazolo[4,3-d]pyrimidine Compound 172

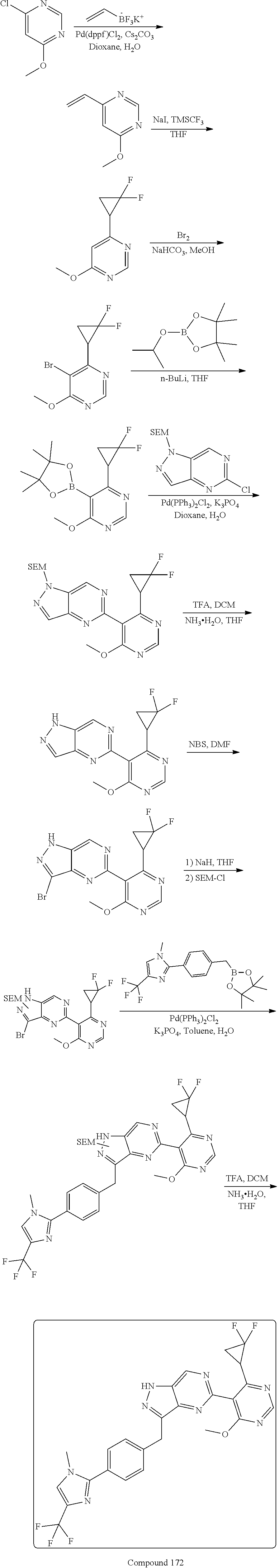

### 1.1 4-methoxy-6-vinyl-pyrimidine

To a solution of 4-chloro-6-methoxy-pyrimidine (9 g, 62.26 mmol) in 1,4-dioxane (260 mL) and water (32 mL) were added cesium carbonate (50.71 g, 155.65 mmol), 1,1′-Bis(diphenylphosphino)ferrocene-palladium(II)dichloride dichloromethane complex (1.02 g, 1.25 μmol) and potassium ethenyltrifluoroborate (16.68 g, 124.52 mmol) at 25° C. The resulted mixture was stirred at 95° C. for 16 hours. The resulted mixture was cooled down to room temperature and the solid was filtered out. The filtrate was concentrated under reduced pressure, then diluted with diethyl ether (200 mL), washed with brine (3×50 mL). The organic layer was dried over anhydrous sodium sulfate. After filtration, the filtrate was concentrated under reduced pressure at 0° C. The residue was purified by silica gel column chromatography, eluted with 0-20% ethyl acetate in petroleum ether to give 4-methoxy-6-vinyl-pyrimidine (7.5 g, 55.09 mmol, 88% yield) as a yellow liquid. MS: m/z=137.30 [M+H]⁺. ¹H NMR (400 MHz, Chloroform-d) δ 8.76 (s, 1H), 6.74-6.62 (m, 214), 6.45 (dd, J=17.2, 1.2 Hz, 1H), 5.65 (dd, J=10.8. 1.2 Hz, 1H), 4.01 (s, 3H).

### 1.2 4-(2,2-difluorocyclopropyl)-6-methoxy-pyrimidine

To a solution of 4-methoxy-6-vinyl-pyrimidine (7.5 g, 55.09 mmol) and sodium iodide (4.13 g, 27.54 mmol) in tetrahydrofuran (64.27 mL) was added trimethyl(trifluoromethyl)silane (31.33 g, 220.34 mmol, 35.01 mL) in one portion. The resulted solution was stirred at 60° C. for 1 hour. Then sodium iodide (825.68 mg, 5.51 mmol) and trimethyl(trifluoromethyl)silane (31.33 g, 220.34 mmol, 35.01 mL) were added and the solution was stirred at 60° C. for 2 hours. The mixture was cooled to room temperature. The resulting mixture was diluted with ethyl acetate (200 mL), washed with brine (3×50 mL). The organic layer was dried over anhydrous sodium sulfate. After filtration, the filtrate was concentrated under reduced pressure. The residue was purified by silica gel column chromatography, eluted with 0-20% ethyl acetate in petroleum ether to give 4-(2,2-difluorocyclopropyl)-6-methoxy-pyrimidine (6.5 g, 34.92 mmol, 63% yield) as a yellow liquid. MS: m/z=187.20 [M+H]⁺. ¹H NMR (400 MHz, Chloroform-d) δ 8.71 (s, 1H), 6.65 (s, 1H), 3.98 (s, 3H), 2.82-2.72 (m, 11H), 2.28-2.18 (m, 11H), 1.92-1.81 (m, 1H). ¹⁹F NMR (376 MHz, Chloroform-d) δ −123.95, −124.35, −142.72, −143.13.

### 1.3 5-bromo-4-(2,2-difluorocyclopropyl)-6-methoxy-pyrimidine

To a solution of 4-(2,2-difluorocyclopropyl)-6-methoxy-pyrimidine (6.5 g, 34.92 mmol) in methanol (50 mL) were added molecular bromine (11.16 g, 69.83 mmol) and sodium bicarbonate (3.52 g, 41.90 mmol) at 25° C. The resulted solution was stirred at 25° C. for 16 hours. The resulting mixture was concentrated under reduced pressure. The reaction was quenched by the addition of saturated sodium thiosulfate aqueous solution (20 mL) at 0° C. The resulting mixture was diluted with ethyl acetate (200 mL), washed with brine (3×30 mL). The organic layer was dried over anhydrous sodium sulfate. After filtration, the filtrate was concentrated under reduced pressure. The residue was purified by silica gel column chromatography, eluted with 1-50% ethyl acetate in petroleum ether to give 5-bromo-4-(2,2-difluorocyclopropyl)-6-methoxy-pyrimidine (6 g, 22.64 μmol, 65% yield) as a yellow liquid. MS: m/z=265.05, 267.05 [M+H]⁺. ¹H NMR (400 MHz, Chloroform-d) δ 8.58 (s, 1H), 4.08 (s, 3H), 3.25-3.17 (m, 1H), 2.55-2.45 (m, 1H), 1.93-1.83 (m, 1H). ¹⁹F NMR (376 MHz, Chloroform-d) δ −123.52, −123.92, −141.82, −142.22.

### 1.4 4-(2,2-difluorocyclopropyl)-6-methoxy-5-(4,4,5,5-tetramethyl-1,3,2-dioxaborolan-2-yl)pyrimidine

To a solution of 5-bromo-4-(2,2-difluorocyclopropyl)-6-methoxy-pyrimidine (1.5 g, 5.66 mmol) and 2-isopropoxy-4,4,5,5-tetramethyl-1,3,2-dioxaborolane (1.58 g, 8.49 mmol, 1.73 mL) in tetrahydrofuran (150 mL) under argon atmosphere was added n-butyllithium (2.5 M in hexane, 6.79 mmol, 2.72 mL) dropwise with stirring at −70° C. The reaction mixture was stirred at −70° C. for 1 hour. The resulted reaction was quenched by saturated ammonium chloride aqueous solution (200 mL), extracted with ethyl acetate (3×200 mL). The combined organic layers were washed with brine (200 mL), dried over anhydrous sodium sulfate. After filtration, the filtrate was concentrated under reduced pressure. The resulted solution was detected by LCMS, could find 70% desired product. The resulted solution was purified by RP-Flash with the following conditions: Column: C18 spherical, 20-30 um, 100A, 40 g; Mobile Phase A: Water (5 mM aq. HCl), Mobile Phase B: MeCN; Flow rate: 50 mL/min; Gradient: 5% B to 5% B in 5 min, 5% B to 25% B in 15 min, 25% B to 25% B in 10 min, 25% B to 95% B in 20 min; Detector: UV 254 & 220 nm; RT: 23 min. The collected fractions were combined and concentrated to give 4-(2,2-difluorocyclopropyl)-6-methoxy-5-(4,4,5,5-tetramethyl-1,3,2-dioxaborolan-2-yl)pyrimidine (400 mg, 1.28 mmol, 22% yield) as a yellow solid. MS: m/z=313.05 [M+H]⁺. ¹H NMR (400 MHz, Chloroform-d) δ 8.77 (s, 1H), 4.03 (s, 3H), 3.21-3.13 (m, 1H), 2.62-2.51 (m, 1H), 1.90-1.72 (m, 1H), 1.42 (s, 12H).

### 1.5 2-[[5-[4-(2,2-difluorocyclopropyl)-6-methoxy-pyrimidin-5-yl]pyrazolo[4,3-d]pyrimidin-1-yl]methoxy]ethyl-trimethyl-silane

To a stirred mixture of 4-(2,2-difluorocyclopropyl)-6-methoxy-5-(4,4,5,5-tetramethyl-1,3,2-dioxaborolan-2-yl)pyrimidine (1 g, 4.35 mmol) and 2-[(5-chloropyrazolo[4,3-d]pyrimidin-1-yl)methoxy]ethyl-trimethyl-silane (1.86 g, 6.52 mmol) in 1,4-dioxane (20 mL) and water (4 mL) was added potassium phosphate (1.85 g, 8.70 mmol) and bis(triphenylphosphine)palladium(II) chloride (304.81 mg, 434.83 mmol) under nitrogen atmosphere at 25° C. The resulting mixture was stirred at 90° C. for 16 hours. The reaction was cooled down to mom temperature, then diluted with water (500 mL), extracted with dichloromethane (3×200 mL). The combined organic layers were washed with brine (200 mL), dried over anhydrous sodium sulfate. After filtration, the filtrate was concentrated under reduced pressure. The residue was purified by silica gel column chromatography, eluted with 52%-55% ethyl acetate in petroleum ether. The collected fractions were combined and concentrated under reduced pressure to give 2-[[5-[4-(2,2-difluorocyclopropyl)-6-methoxy-pyrimidin-5-yl]pyrazolo[4,3-d]pyrimidin-1-yl]methoxy]ethyl-trimethyl-silane (500 mg, 1.15 mmol, 26% yield) as an off-white solid. MS: m/z=435.20 [M+H]+. ¹H NMR (400 MHz, Chloroform-d) δ 9.43 (s, 1H), 8.82 (s, 1H), 8.36 (s, 1H), 5.90 (s, 2H), 4.00 (s, 3H), 3.71-3.62 (m, 2H), 2.73-2.45 (m, 2H), 1.85-1.59 (m, 1H), 1.00-0.94 (m, 2H), 0.00 (s, 9H).

### 1.6 5-[4-(2,2-difluorocyclopropyl)-6-methoxy-pyrimidin-5-yl]-1H-pyrazolo[4,3-d]pyrimidine

To a stirred solution of 2-[[5-[4-(2,2-difluorocyclopropyl)-6-methoxy-pyrimidin-5-yl]pyrazolo[4,3-d]pyrimidin-1-yl]methoxy]ethyl-trimethyl-silane (500 mg, 1.15 mmol) in chloroform (5 mL) was trifluoroacetic acid (5 mL) and then stirred for 2 hours at 25° C. The resulted mixture was concentrated under reduced pressure, then tetrahydrofuran (5 mL) and ammonium hydroxide (5 mL, 28% aqueous solution) were added in the above mixture. The resulted mixture was stirred for 30 min at 25° C. The resulted mixture was concentrated under reduced pressure. The residue was purified by silica gel column chromatography, eluted with 50%-60% ethyl acetate in petroleum ether to afford 5-[4-(2,2-difluorocyclopropyl)-6-methoxy-pyrimidin-5-yl]-1H-pyrazolo[4,3-d]pyrimidine (250 mg, 822 μmol, 71% yield) as an off-white solid. MS: m/z=305.10 [M+H]⁺. ¹H NMR (400 MHz, Chloroform-d) δ 9.43 (s, 1H), 8.85 (s, 1H), 8.47 (s, 1H), 4.01 (s, 3H), 2.73-2.65 (m, 1H), 2.58-2.50 (m, 1H), 1.79-1.74 (m, 1H).

### 1.7 3-bromo-5-[4-(2,2-difluorocyclopropyl)-6-methoxy-pyrimidin-5-yl]-1H-pyrazolo[4,3-d]pyrimidine

To a stirred mixture of 5-[4-(2,2-difluorocyclopropyl)-6-methoxy-pyrimidin-5-yl]-1H-pyrazolo[4,3-d]pyrimidine (250 mg, 822 μmol) in N,N-dimethylformamide (5 mL) was added N-Bromosuccinimide (219 mg, 1.23 mmol) under nitrogen atmosphere at 25° C. The resulting mixture was stirred for 2 hours at 25° C. The reaction was quenched by the addition of saturated ammonium chloride aqueous solution (100 mL). The resulted mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 45%-50% ethyl acetate in petroleum ether to give 3-bromo-5-[4-(2,2-difluorocyclopropyl)-6-methoxy-pyrimidin-5-yl]-1H-pyrazolo[4,3-d]pyrimidine (240 mg, 626.39 μmol, 76% yield) as a yellow oil. MS: m/z=382.95, 384.95 [M+H]⁺. ¹H NMR (400 MHz, Chloroform-d) δ 11.01 (s, 1H), 9.36 (s, 1H), 8.83 (s, 1H), 3.99 (s, 3H), 2.71-2.63 (m, 1H), 2.56-2.48 (m, 1H), 1.79-1.74 (m, 1H).

### 1.8 3-bromo-5-(4-(2,2-difluorocyclopropyl)-6-methoxypyrimidin-5-yl)-1-((2-(trimethylsilyl)ethoxy)methyl)-1H-pyrazolo[4,3-d]pyrimidine & 3-bromo-5-(4-(2,2-difluorocyclopropyl)-6-methoxypyrimidin-5-yl)-2-((2-(trimethylsilyl)ethoxy)methyl)-2H-pyrazolo[4,3-d]pyrimidine

To a stirred mixture of 3-bromo-5-[4-(2,2-difluorocyclopropyl)-6-methoxy-pyrimidin-5-yl]-1H-pyrazolo[4,3-d]pyrimidine (240 mg, 626 μmol) in tetrahydrofuran (5 mL) was added sodium hydride (30 mg, 752 μmol, 60% purity) under nitrogen atmosphere at 0° C. The resulting mixture was stirred for 1 hour at 0° C. To the above mixture was added (2-(chloromethoxy)ethyl)trimethylsilane (125 mg, 752 μmol) under nitrogen atmosphere at 0° C. The resulted mixture was stirred at 25° C. for 2 hours. The reaction was quenched by the addition of saturated ammonium chloride aqueous solution (100 mL). The resulting mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 32%-33% ethyl acetate in petroleum ether to give a mixture of 3-bromo-5-(4-(2,2-difluorocyclopropyl)-6-methoxypyrimidin-5-yl)-1-((2-(trimethylsilyl)ethoxy)methyl)-1H-pyrazolo[4,3-d]pyrimidine & 3-bromo-5-(4-(2,2-difluorocyclopropyl)-6-methoxypyrimidin-5-yl)-2-((2-(trimethylsilyl)ethoxy)methyl)-2H-pyrazolo[4,3-d]pyrimidine (mixture of two isomers, 260 mg, 506.42 μmol, 81% yield) as a yellow oil. MS: m/z=513.10, 515.10 [M+H]⁺. ¹H NMR (400 MHz, Chloroform-d) δ 9.60, 9.42 (2s, 1H), 8.84 (s, 1H), 5.96, 5.86 (2s, 2H), 4.01 (s, 3H), 3.82-3.70 (m, 2H), 2.68-2.50 (m, 2H), 1.80-1.71 (m, 1H), 1.01-0.96 (m, 2H), 0.02 (s, 9H).

### 1.9 5-(4-(2,2-difluorocyclopropyl)-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-(2,2-difluorocyclopropyl)-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine

To a stirred solution of the mixture of 3-bromo-5-(4-(2,2-difluorocyclopropyl)-6-methoxypyrimidin-5-yl)-1-((2-(trimethylsilyl)ethoxy)methyl)-1H-pyrazolo[4,3-d]pyrimidine & 3-bromo-5-(4-(2,2-difluorocyclopropyl)-6-methoxypyrimidin-5-yl)-2-((2-(trimethylsilyl)ethoxy)methyl)-2H-pyrazolo[4,3-d]pyrimidine (260 mg, 506 μmol) in toluene (5 mL) and water (1 mL) were added bis(triphenylphosphine)palladium(II) chloride (107 mg, 152 μmol) and potassium phosphate (215 mg, 1.01 mmol) under nitrogen atmosphere at 25° C. The resulting mixture was stirred at 90° C. for 16 hours. The reaction was cooled down to room temperature, then quenched with water (100 mL), extracted with dichloromethane (3×50 mL). The combined organic layers were washed with brine (100 mL), dried over anhydrous sodium sulfate. After filtration, the filtrate was concentrated under reduced pressure. The residue was purified by silica gel column chromatography, eluted with 50%-60% ethyl acetate in petroleum ether to give a mixture of 5-(4-(2,2-difluorocyclopropyl)-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-(2,2-difluorocyclopropyl)-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (80 mg, 118.92 μmol, 23% yield) as a yellow solid. MS: m/z=673.35 [M+H]⁺.

### 1.10 5-[4-(2,2-difluorocyclopropyl)-6-methoxy-pyrimidin-5-yl]-3-[[4-[1-methyl-4-(trifluoromethyl)imidazol-2-yl]phenyl]methyl]-1H-pyrazolo[4,3-d]pyrimidine

To a stirred solution of 5-(4-(2,2-difluorocyclopropyl)-6-methoxypyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-(2,2-difluorocyclopropyl)-6-methoxypyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (80 mg, 119 μmol) in methylene chloride (1 mL) was added trifluoroacetic acid (1 mL) and then stirred for 2 hours at 25° C. The resulted mixture was concentrated under reduced pressure, then tetrahydrofuran (1 mL) and ammonia water (1 mL) were added in the above mixture, the resulted mixture was stirred for 30 min at 25° C. The resulted mixture was concentrated under reduced pressure. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 65% B in 20 min, 65% B to 65% B in 4 min, 65% B to 95% B in 5 min; Detector: UV 254& 210 nm; RT: 25 min. The collected fractions were combined, concentrated and then lyophilized to give 5-[4-(2,2-difluorocyclopropyl)-6-methoxy-pyrimidin-5-yl]-3-[[4-[1-methyl-4-(trifluoromethyl)imidazol-2-yl]phenyl]methyl]-1H-pyrazolo[4,3-d]pyrimidine (27.5 mg, 50.69 μmol, 43% yield) as an off-white solid. MS: m/z=543.15 [M+H]⁺. ¹H NMR (400 MHz, Chloroform-d) δ 9.24 (s, 1H), 8.82 (s, 1H), 7.65 (d, J=8.0 Hz, 2H), 7.56 (d, J=8.0 Hz, 2H), 7.40 (s, 1H), 4.54 (s, 2H), 3.99 (s, 3H), 3.81 (s, 3H), 2.72-2.43 (m, 2H), 1.70-1.61 (m, 1H). ¹⁹F NMR (376 MHz, Chloroform-d) δ −62.18, −123.82, −124.22, −141.78, −142.18.

### Example 97. rel-(S)-[2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol & rel-(R)-[2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol Compound 103 & Compound 141

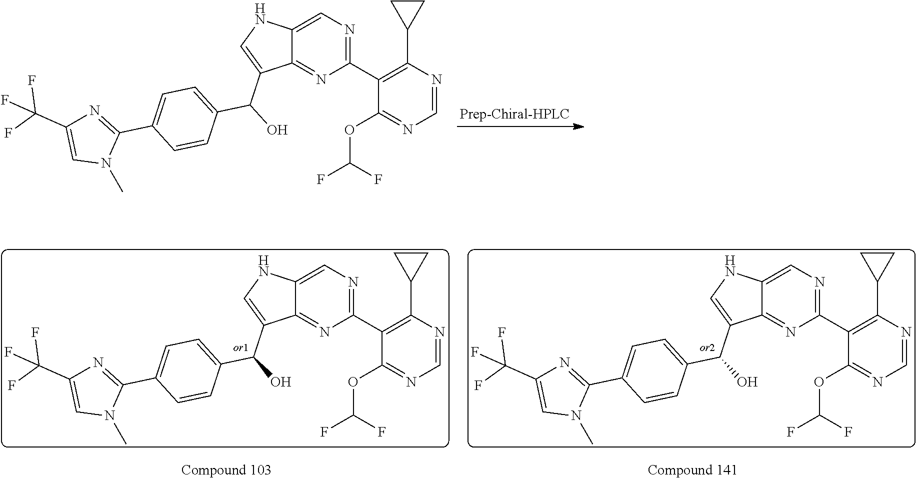

### 1.1 rel-(S)-[2-[4-cyclopropyl-6-(difluoromethoxy)pyrimidin-5-yl]-5H-pyrrolo[3,2-]pyrimidin-7-yl]-[4-[1-methyl-4-(trifluoromethyl)imidazol-2-yl]phenyl]methanol & rel-(R)-[2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol

The racemic of (2-(4-cyclopropyl-6-(difluoromethoxy)pyrimidin-5-yl)-5H-pyrrolo[3,2-d]pyrimidin-7-yl)(4-(1-methyl-4-(trifluoromethyl)-1H-imidazol-2-yl)phenyl)methanol (140 mg) was separated by Prep-Chiral-HPLC with the following conditions: Column: Lux 5 um Cellulose-4, 2.12×25 cm, 5 μm; Mobile Phase A: Hex (0.5% 2 M NH₃-MeOH), Mobile Phase B: MeOH:EtOH=1:1; Flow rate: 20 mL/min; Gradient: 20% B to 20% B in 11 min; Detector: UV 254 & 210 nm; Sample Solvent: MeOH:EtOH=1:1; Injection Volume: 0.6 mL; Number Of Runs: 7.

The fractions with shorter retention time (RT: 7.45 min) was concentrated under reduced pressure and then lyophilized overnight to afford rel-(S)-[2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol (53.7 mg, 96.33 μmol) as a white solid. MS: m/z =558.25 [M+H]⁺. ¹H NMR (400 MHz, Chloroform-d) δ 11.06 (s, 1H), 8.83 (s, 1H), 8.64 (s, 1H), 7.68-7.29 (m, 6H), 7.08 (s, 1H), 6.33 (s, 1H), 3.74 (s, 3H), 1.91-1.81 (m, 1H), 1.30-1.18 (m, 2H), 1.00-0.85 (m, 2H). ¹⁹F NMR (376 MHz, Chloroform-d) δ −62.36, −89.37.

The fractions with longer retention time (RT: 9.27 min) was concentrated under reduced pressure and then lyophilized overnight to afford rel-(R)-[2-[4-cyclopropyl-6-(difluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol (57.3 mg, 102.79 μmol) as a white solid. MS: m/z=558.25 [M+H]⁺. ¹H NMR (400 MHz, Chloroform-d) δ 11.11 (s, 1H), 8.82 (s, 1H), 8.62 (s, 1H), 7.68-7.28 (m, 6H), 7.03 (d, J=2.0 Hz, 1H), 6.31 (s, 1H), 3.73 (s, 3H), 1.90-1.81 (m, 1H), 1.29-1.19 (m, 2H), 0.98-0.88 (m, 2H). ¹⁹F NMR (376 MHz, Chloroform-d) δ −62.34, −89.36.

### Example 98. 5-[4-cyclopropyl-6-(trideuteriomethoxy)pyrimidin-5-yl]-3-[[4-[1-methyl-4-(trifluoromethyl)imidazol-2-yl]phenyl]methyl]-1H-pyrazolo[4,3-d]pyrimidine Compound 163

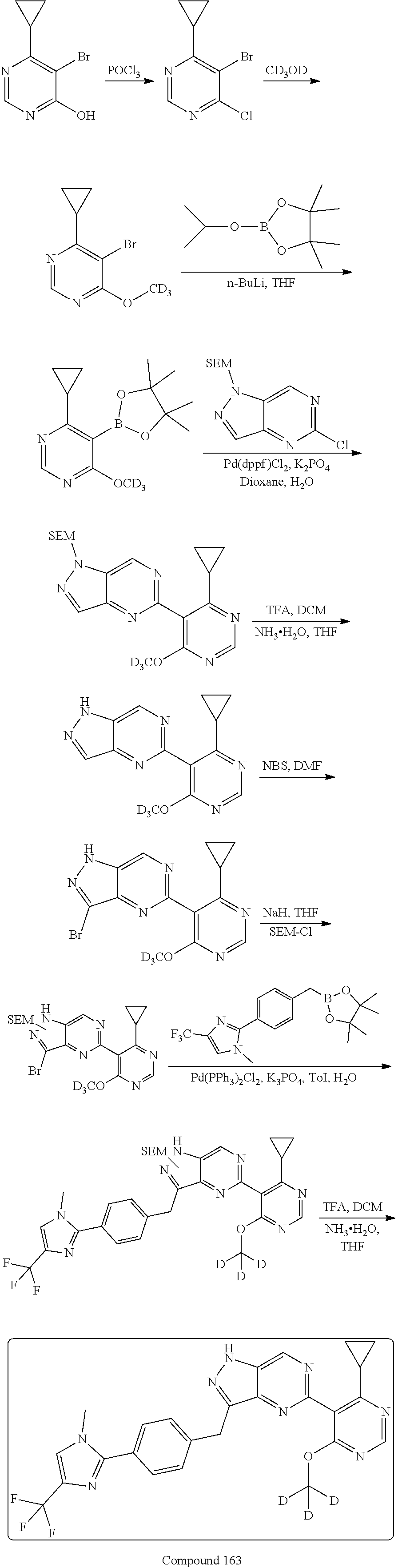

### 1.1 5-bromo-4-chloro-6-cyclopropylpyrimidin

Added 5-bromo-6-cyclopropyl-pyrimidin-4-ol (5 g, 23.25 mmol) to phosphoryl trichloride (3.57 g, 23.25 mmol, 10 mL) at 25° C. under nitrogen atmosphere. The mixture was stirred for 20 min at 80° C. under nitrogen atmosphere. The phosphoryl trichloride was removed under reduced pressure and the residue evaporated from toluene to give crude product which was brown solid (5.05 g, 93% yield).

### 1.2 5-bromo-4-cyclopropyl-6-(trideuteriomethoxy)pyrimidine

The obtained crude product (3 g) was added 10% deuterated sodium methanolate in deuterated methanol (15 mL) at 25° C. under nitrogen atmosphere. The resulting mixture was concentrated under reduced pressure. The residue was purified by silica gel column chromatography, eluted with 1%-35% ethyl acetate in petroleum ether to give 5-bromo-4-cyclopropyl-6-(trideuteriomethoxy)pyrimidine (2.58 g, 2.05 mmol, 44% yield) as a white solid. MS: m/z=231.95, 233.95 [M+H]⁺. ¹H NMR (400 MHz, Chloroform-d) δ 8.46 (s, 1H), 2.59-2.51 (m, 1H), 1.23-1.19 (m, 2H), 1.18-1.09 (m, 2H).

### 1.3 4-cyclopropyl-5-(4,4,5,5-tetramethyl-1,3,2-dioxaborolan-2-yl)-6-(trideuteriomethoxy)pyrimidine

To a stirred solution of 5-bromo-4-cyclopropyl-6-(trideuteriomethoxy)pyrimidine (2.58 g, 11.12 mmol) and 2-isopropoxy-4,4,5,5-tetramethyl-1,3,2-dioxaborolane (2.07 g 11.12 mmol) in tetrahydrofuran (5 mL) was added n-butyllithium (2.5M in hexane, 6.67 mL, 16.68 mmol) dropwise over 5 min at −78° C. under nitrogen atmosphere. The resulting mixture was stirred for 1.5 hours at −78° C. under nitrogen atmosphere. The reaction was monitored by LCMS. The reaction was quenched by the addition of saturated ammonium chloride aqueous (20 mL) at 0° C. The resulting mixture was extracted with ethyl acetate (3×20 mL). The combined organic layers were washed with brine (10 mL), dried over anhydrous sodium sulfate. After filtration, the filtrate was concentrated under reduced pressure. The residue was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 95% B in 20 min; Detector: UV 254 & 210 nm; RT: 15.2 min. The collected fractions were combined, concentrated under reduced pressure and lyophilized to give 4-cyclopropyl-5-(4,4,5,5-tetramethyl-1,3,2-dioxaborolan-2-yl)-6-(trideuteriomethoxy)pyrimidine (931 mg, 3.34 mmol, 29% yield) as a white solid. MS: m/z=280.05 [M+H]⁺.

### 1.4 2-[[5-[4-cyclopropyl-6-(trideuteriomethoxy)pyrimidin-5-yl]pyrazolo[4,3-d]pyrimidin-1-yl]methoxy]ethyl-trimethyl-silane

To a stirred solution of 4-cyclopropyl-5-(4,4,5,5-tetramethyl-1,3,2-dioxaborolan-2-yl)-6-(trideuteriomethoxy)pyrimidine (93 mg, 3.34 mmol) and 2-[(5-chloropyrazolo[4,3-d]pyrimidin-1-yl)methoxy]ethyl-trimethyl-silane (1425 mg, 5.01 mmol) in water (0.4 mL) and 1,4-dioxane (2 mL) were added 1,1′-bis(diphenylphosphino)ferrocene-palladium(II)dichloride dichloromethane complex (545 mg, 667 μmol) and potassium phosphate (509 mg, 2.40 mmol) at 25° C. under nitrogen atmosphere. The resulting mixture was stirred for 16 hours at 100° C. under nitrogen atmosphere. The reaction was monitored by LCMS. The resulting mixture was concentrated under reduced pressure. The residue was purified by silica gel column chromatography, eluted with 1%-50% ethyl acetate in petroleum ether to give 2-[[5-[4-cyclopropyl-6-(trideuteriomethoxy)pyrimidin-5-yl]pyrazolo[4,3-d]pyrimidin-1-yl]methoxy]ethyl-trimethyl-silane (80 mg, 837.79 μmol, 25% yield) as a yellow solid. MS: m, =402.15 [M+H]⁺. ¹H NMR (400 MHz, Chloroform-d) δ 9.43 (s, 1H), 8.71 (s, 1H), 8.35 (s, 1H), 5.87 (s, 2H), 3.71-3.64 (m, 2H), 1.74-1.63 (m, 1H), 1.30-1.25 (m, 2H), 1.02-0.81 (m, 4H).

### 1.5 5-[4-cyclopropyl-6-(trideuteriomethoxy)pyrimidin-5-yl]-1H-pyrazolo[4,3-d]pyrimidine

To a stirred solution of 2-[[5-[4-cyclopropyl-6-(trideuteriomethoxy)pyrimidin-5-yl]pyrazolo[4,3-d]pyrimidin-1-yl]methoxy]ethyl-trimethyl-silane (334 mg, 831.77 μmol) in dichloromethane (2 mL) was added 2,2,2-trifluoroacetic acid (2.96 g, 25.96 mmol, 2 mL) at 25° C. The resulting mixture was stirred for 1 hour at 25° C. The resulting mixture was concentrated under reduced pressure. To a stirred solution of the residue in tetrahydrofuran (2 mL) was added ammonium hydroxide (28% ammonia aqueous solution, 2 mL) at 25° C. The resulting mixture was stirred for 1 hours at 25° C. The reaction was monitored by LCMS. The resulting mixture was concentrated under reduced pressure. The residue was purified by silica gel column chromatography, eluted with 1%-70% ethyl acetate in petroleum ether to give 5-[4-cyclopropyl-6-(trideuteriomethoxy)pyrimidin-5-yl]-1H-pyrazolo[4,3-d]pyrimidine (126 mg, 464.44 μmol, 55% yield) as a yellow solid. MS: m, =272.05 [M+H]⁺. ¹H NMR (400 MHz, Chloroform-d) δ 9.40 (s, 1H), 8.78 (s, 1H), 8.47 (s, 1H), 1.75-1.69 (m, 1H), 1.42-1.39 (m, 2H), 1.02-0.99 (m, 2H).

### 1.6 3-bromo-5-[4-cyclopropyl-6-(trideuteriomethoxy)pyrimidin-5-yl]-1H-pyrazolo[4,3-d]pyrimidine

To a stirred solution of and 5-[4-cyclopropyl-6-(trideuteriomethoxy)pyrimidin-5-yl]-1H-pyrazolo[4,3-d]pyrimidine (126 mg, 464 μmol) in N,N-dimethylformamide (1.2 mL) was added 1-bromopyrrolidine-2,5-dione (124 mg, 697 μmol) at 25° C. under nitrogen atmosphere. The resulting mixture was stirred for 16 hours at 25° C. under nitrogen atmosphere. The reaction was monitored by LCMS. The resulting mixture was diluted with water (10 mL). The resulting mixture was extracted with ethyl acetate (1×20 mL). The combined organic layers were washed with brine (20 mL), dried over anhydrous sodium sulfate. After filtration, the filtrate was concentrated under reduced pressure. The residue was purified by silica gel column chromatography, eluted with 1%-60% ethyl acetate in petroleum ether to give 3-bromo-5-[4-cyclopropyl-6-(trideuteriomethoxy)pyrimidin-5-yl]-1H-pyrazolo[4,3-d]pyrimidine (142 mg, 405.50 μmol, 87% yield) as a yellow solid. MS: m/z=349.95, 351.95 [M+H]⁺. ¹H NMR (400 MHz, DMSO-d₆) δ 14.44 (s, 1H), 9.53 (s, 1H), 8.72 (s, 1H), 1.60-1.53 (m, 1H), 1.09-1.05 (m, 2H), 0.94-0.85 (m, 2H).

### 1.7 3-bromo-5-(4-cyclopropyl-6-(methoxy-d3)pyrimidin-5-yl)-2-((2-(trimethylsilyl)ethoxy)methyl)-2H-pyrazolo[4,3-d]pyrimidine & 3-bromo-5-(4-cyclopropyl-6-(methoxy-d3)pyrimidin-5-yl)-1-((2-(trimethylsilyl)ethoxy)methyl)-1H-pyrazolo[4,3-d]pyrimidine

To a stirred solution of 3-bromo-5-[4-cyclopropyl-6-(trideuteriomethoxy)pyrimidin-5-yl]-1H-pyrazolo[4,3-d]pyrimidine (142 mg, 405.5 μmol) in tetrahydrofuran (1.5 mL) was added sodium hydride (60% dispersion in mineral oil, 20 mg, 507 μmol) in portions over 5 min at 0° C. under nitrogen atmosphere. After 1 hours, the resulting mixture was added 2-(chloromethoxy)ethyl-trimethyl-silane (84.5 mg, 507 μmol, 90 μL) at 0° C. under nitrogen atmosphere. The resulting mixture was stirred for 3 hours at 25° C. under nitrogen atmosphere. The reaction was monitored by LCMS. The reaction was quenched by the addition of saturated ammonium chloride (8 mL) aqueous at 0° C. The resulting mixture was extracted with ethyl acetate (3×10 mL). The combined organic layers were washed with brine (8 mL), dried over anhydrous sodium sulfate. After filtration, the filtrate was concentrated under reduced pressure. The residue was purified by silica gel column chromatography, eluted with 50% ethyl acetate in petroleum ether to give the mixture of 3-bromo-5-(4-cyclopropyl-6-(methoxy-d3)pyrimidin-5-yl)-2-((2-(trimethylsilyl)ethoxy)methyl)-2H-pyrazolo[4,3-d]pyrimidine & 3-bromo-5-(4-cyclopropyl-6-(methoxy-d3)pyrimidin-5-yl)-1-((2-(trimethylsilyl)ethoxy)methyl)-1H-pyrazolo[4,3-d]pyrimidine (129 mg, 268.50 μmol, 66% yield) as a yellow solid. MS: m/z=480.05, 482.05 [M+H]⁺.

### 1.8 5-(4-cyclopropyl-6-(methoxy-d3)pyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine & 5-(4-cyclopropyl-6-(methoxy-d3)pyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine

To a stirred solution of the mixture of 3-bromo-5-(4-cyclopropyl-6-(methoxy-d3)pyrimidin-5-yl)-2-((2-(trimethylsilyl)ethoxy)methyl)-2H-pyrazolo[4,3-d]pyrimidine & 3-bromo-5-(4-cyclopropyl-6-(methoxy-d3)pyrimidin-5-yl)-1-((2-(trimethylsilyl)ethoxy)methyl)-1H-pyrazolo[4,3-d]pyrimidine in toluene (1.5 mL) and water (0.3 mL) were added 1-methyl-2-[4-[(4,4,5,5-tetramethyl-1,3,2-dioxaborolan-2-yl)methyl]phenyl]-4-(trifluoromethyl)imidazole (98 mg, 269 μmol), bis(triphenylphosphine)palladium(II) chloride (57 mg, 81 μmol) and potassium phosphate (171 mg, 806 μmol) at 25° C. under nitrogen atmosphere. The resulting mixture was stirred for 16 hours at 90° C. under nitrogen atmosphere. The reaction was monitored by LCMS. The resulting mixture was filtered, the filter cake was washed with ethyl acetate (3×3 mL). The filtrate was concentrated under reduced pressure. The residue was purified by silica gel column chromatography, eluted with 1%-60% ethyl acetate in petroleum ether to give 5-(4-cyclopropyl-6-(methoxy-d3)pyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine & 5-(4-cyclopropyl-6-(methoxy-d3)pyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine (70 mg, 109.42 μmol, 40% yield, mixture of two isomers) as a yellow solid. MS: m/z=640.30 [M+H]⁺.

### 1.9 5-[4-cyclopropyl-6-(trideuteriomethoxy)pyrimidin-5-yl]-3-[[4-[1-methyl-4-(trifluoromethyl)imidazol-2-yl]phenyl]methyl]-1H-pyrazolo[4,3-d]pyrimidine

To a stirred solution of 5-(4-cyclopropyl-6-(methoxy-d3)pyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine & 5-(4-cyclopropyl-6-(methoxy-d3)pyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine (70 mg, 109.42 μmol, mixture of two isomers) in dichloromethane (2 mL) was added 2,2,2-trifluoroacetic acid (2.96 g, 25.96 mmol, 2 mL) at 25° C. The resulting mixture was stirred for 1 hour at 25° C. The resulting mixture was concentrated under reduced pressure. To a stirred solution of the residue in tetrahydrofuran (2 mL) was added ammonium hydroxide (28% ammonia aqueous solution, 2 mL) at 25° C. The resulting mixture was stirred for 1 hour at 25° C. The reaction was monitored by LCMS. The resulting mixture was concentrated under reduced pressure. The residue was purified by silica gel column chromatography, eluted with 1%-80% ethyl acetate in petroleum ether to give crude product. The obtained crude product was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 95% B in 20 min; Detector: UV 254 & 210 nm; RT: 13 min. The collected fractions were combined, concentrated under reduced pressure and lyophilized to afford 5-[4-cyclopropyl-6-(trideuteriomethoxy)pyrimidin-5-yl]-3-[[4-[1-methyl-4-(trifluoromethyl)imidazol-2-yl]phenyl]methyl]-1H-pyrazolo[4,3-d]pyrimidine (16.2 mg, 31.80 μmol, 26% yield) as a white solid. MS: m/z=510.25 [M+H]⁺. ¹H NMR (400 MHz, Chloroform-d) δ 9.25 (s, 1H), 8.75 (s, 1H), 7.64 (d, J=8.4 Hz, 2H), 7.59 (d, J=8.4 Hz, 2H), 7.37 (s, 1H), 4.55 (s, 2H), 3.80 (s, 3H), 1.73-1.65 (m, 1H), 1.35-1.31 (m, 2H), 0.92-0.89 (m, 2H). ¹⁹F NMR (376 MHz, CDCl₃) δ −62.44.

### Example 99. 2-[4-cyclopropyl-6-(trideuteriomethoxy)pyrimidin-5-yl]-7-[[4-[1-methyl-4-(trifluoromethyl)imidazol-2-yl]phenyl]methyl]-5H-pyrrolo[3,2-d]pyrimidine Compound 204

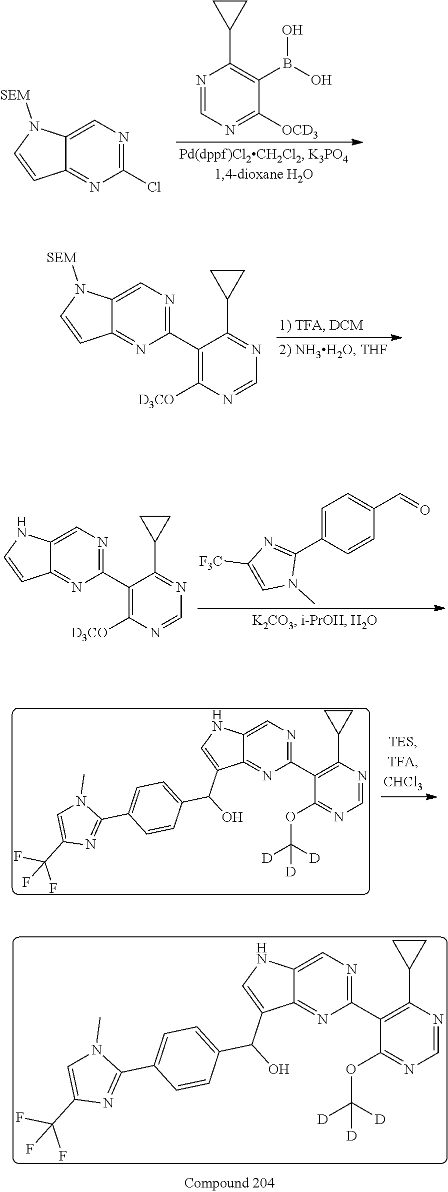

### 1.1 2-[[2-[4-cyclopropyl-6-(trideuteriomethoxy)pyrimidin-5-yl]pyrrolo[3,2-d]pyrimidin-5-yl]methoxy]ethyl-trimethyl-silane

To a stirred solution of 2-[(2-chloropyrrolo[3,2-d]pyrimidin-5-yl)methoxy]ethyl-trimethyl-silane (203.35 mg, 716.44 μmol) and 4-cyclopropyl-5-(4,4,5,5-tetramethyl-1,3,2-dioxaborolan-2-yl)-6-(trideuteriomethoxy)pyrimidine (200 mg, 716.4 μmol) in 1,4-dioxane (2 mL) and water (0.4 mL) were added cyclopentyl(diphenyl)phosphane:dichloromethane:dichloropalladium;iron (59 mg, 71.6 μmol) and potassium phosphate (456 mg, 2.15 mmol) at 25° C. under nitrogen atmosphere. The resulting mixture was stirred at 100° C. for 16 hours. The reaction was monitored by LCMS. The resulting mixture was cooled down to room temperature and the solid was filtered out. The filtrate was concentrated under reduced pressure. The residue was purified by silica gel column chromatography, eluted with 1%-60% ethyl acetate in petroleum ether to afford 2-[[2-[4-cyclopropyl-6-(trideuteriomethoxy)pyrimidin-5-yl]pyrrolo[3,2-d]pyrimidin-5-yl]methoxy]ethyl-trimethyl-silane (230 mg, 574.19 μmol, 80% yield) as a yellow oil. MS: m/z=401.15.

### 1.2 2-[4-cyclopropyl-6-(trideuteriomethoxy)pyrimidin-5-yl]-5H-pyrrolo[3,2-d]pyrimidine

To a solution of 2-[[2-[4-cyclopropyl-6-(trideuteriomethoxy)pyrimidin-5-yl]pyrrolo[3,2-d]pyrimidin-5-yl]methoxy]ethyl-trimethyl-silane (375 mg, 936.18 μmol) in dichloromethane (2 mL) was added 2,2,2-trifluoroacetic acid (2.96 g, 25.96 mmol, 2 mL) dropwise with stirring at 0° C., and then stirred at 25° C. for 2 hours. The reaction progress was monitored by TLC. The resulted solution was concentrated under reduced pressure. To a solution of the residue in tetrahydrofuran (2 mL) was added ammonium hydroxide (2 mL) dropwise with stirring at 0° C. and then stirred at 25° C. for 0.5 hours. The reaction progress was monitored by LCMS. The resulted solution was concentrated under reduced pressure. The residue was purified by silica gel column chromatography, eluted with 1%-9% methanol in dichloromethane to afford 2-[4-cyclopropyl-6-(trideuteriomethoxy)pyrimidin-5-yl]-5H-pyrrolo[3,2-d]pyrimidine (164 mg, 606.72 μmol, 64% yield) as a yellow oil. MS: m/z=271.10.

### 1.3 [2-[4-cyclopropyl-6-(trideuteriomethoxy)pyrimidin-5-yl]-5H-pyrrolo[3,2-d]pyrimidin-7-yl]-[4-[1-methyl-4-(trifluoromethyl)imidazol-2-yl]phenyl]methanol

To a mixture of 2-[4-cyclopropyl-6-(trideuteriomethoxy)pyrimidin-5-yl]-5H-pyrrolo[3,2-d]pyrimidine (164 mg, 606.72 μmol) and 4-[1-methyl-4-(trifluoromethyl)imidazol-2-yl]benzaldehyde (185 mg, 728 μmol) in isopropanol (1 mL) were added potassium carbonate (168 mg, 1.2 mmol, 73 μL) and water (I mL) at 25° C. under nitrogen atmosphere and then stirred at 50° C. for 16 hours. The reaction progress was monitored by LCMS. The resulting mixture was cooled to room temperature and diluted with water (5 mL), then extracted with ethyl acetate (3×10 mL). The combined organic layers were concentrated under reduced pressure. The residue was purified by Prep-TLC, eluted with 9% methanol in dichloromethane to give the crude product. The crude product was purified by RP-Flash chromatography with the following conditions: Column: C18 spherical, 20-30 um, 100A, 40 g; Mobile Phase A: Water (10 mM aq. NH₄HCO₃), Mobile Phase B: acetonitrile; Flow rate: 35 ml/min; Gradient: 2% B to 2% B in 5 min. 2% B to 34% B in 14 min; 34% B to 34% B in 2.7 min; 34% B to 98% B in 5 min; 98% B to 98% B in 5 min; Detector: UV 254 & 210 nm; RT: 20 min. The collected fractions were combined, concentrated and then lyophilized to give [2-[4-cyclopropyl-6-(trideuteriomethoxy)pyrimidin-5-yl]-5H-pyrrolo[3,2-d]pyrimidin-7-yl]-[4-[1-methyl-4-(trifluoromethyl)imidazol-2-yl]phenyl]methanol (62.3 mg, 118.78 μmol, 20% yield) as an off-white solid. MS: m/z=525.25 [M+H]⁺. ¹H NMR (400 MHz, Chloroform-d) δ 10.97 (s, 1H), 8.95 (s, 1H), 8.66 (s, 1H), 7.66 (d, J=8.4 Hz, 2H), 7.61 (d, J=8.4 Hz, 2H), 7.38 (s, 1H), 7.24 (s, 1H), 6.41 (s, 1H), 3.78 (s, 3H), 1.79-1.73 (m, 1H), 1.28-1.17 (m, 2H), 0.95-0.83 (m, 2H). ¹⁹F NMR (377 MHz, Chloroform-d) δ −62.49.

### 1.4 2-[4-cyclopropyl-6-(trideuteriomethoxy)pyrimidin-5-yl]-7-[[4-[1-methyl-4-(trifluoromethyl)imidazol-2-yl]phenyl]methyl]-5H-pyrrolo[3,2-d]pyrimidine

To a solution of [2-[4-cyclopropyl-6-(trideuteriomethoxy)pyrimidin-5-yl]-5H-pyrrolo[3,2-d]pyrimidin-7-yl]-[4-[1-methyl-4-(trifluoromethyl)imidazol-2-yl]phenyl]methanol (132 mg, 251.85 μmol) and triethylsilane (2 mL) in chloroform (2 mL) was added 2,2,2-trifluoroacetic acid (1 mL) at 0° C., and then stirred at 25° C. for 16 hours. The reaction progress was monitored by LCMS. The resulting mixture was concentrated under reduced pressure. The residue was purified by Prep-TLC, eluted with 11% methanol in dichloromethane to give the crude product. The crude product was purified by RP-Flash chromatography with the following conditions: Column: C18 spherical, 20-30 um, 100A, 40 g; Mobile Phase A: Water (10 mM aq. NH₄HCO₃), Mobile Phase B: MeCN; Flow rate: 35 mL/min; Gradient: 2% B to 2% B in 5 min, 2% B to 28% B in 16 min; 28% B to 28% B in 1.8 min; 28% B to 98% B in 5 min; 98% B to 98% B in 5 min; Detector: UV 254 & 210 nm; RT: 22 min. The collected fractions were combined, concentrated and then lyophilized to give 2-[4-cyclopropyl-6-(trideuteriomethoxy)pyrimidin-5-yl]-7-[[4-[1-methyl-4-(trifluoromethyl)imidazol-2-yl]phenyl]methyl]-5H-pyrrolo[3,2-d]pyrimidine (62.3 mg, 122.51 μmol, 48% yield) as an off-white solid. MS: m/z=509.20 [M+H]⁺. ¹H NMR (400 MHz, Chloroform-d) δ 10.73 (s, 1H), 8.96 (s, 1H), 8.65 (s, 1H), 7.53 (d, J=8.0 Hz, 2H), 7.42 (d, J=8.4 Hz, 2H), 7.37 (s, 1H), 7.19 (s, 1H), 4.28 (s, 2H), 3.78 (s, 3H), 1.75-1.69 (m, 1H), 1.23-1.19 (m, 2H), 0.86-0.81 (m, 2H). ¹⁹F NMR (377 MHz, Chloroform-d) δ −62.44.

### Example 100. 2-(4-cyclopropyl-6-methoxy-2-methylpyrimidin-5-yl)-7-(4-(1-methyl-4-(trifluoromethyl)-1H-imidazol-2-yl)benzyl)-5H-pyrrolo[3,2-d]pyrimidine Compound 142

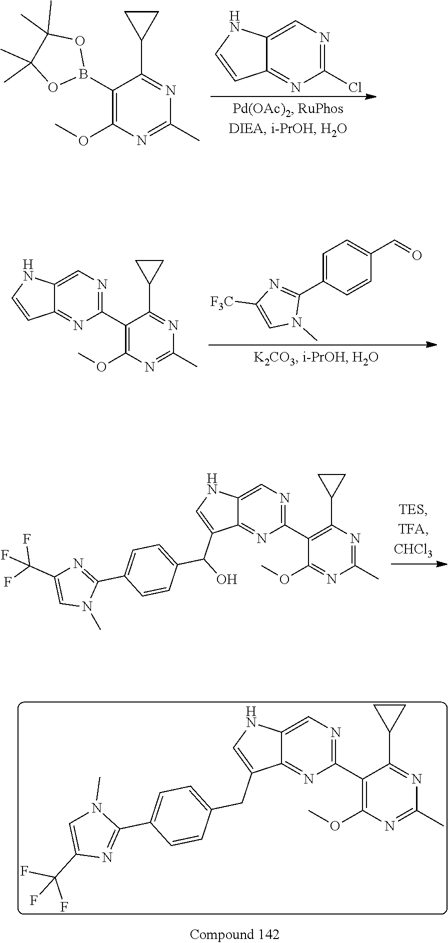

### 1.1 2-(4-cyclopropyl-6-methoxy-2-methylpyrimidin-5-yl)-5H-pyrrolo[3,2-d]pyrimidine

To a solution of 4-cyclopropyl-6-methoxy-2-methyl-5-(4,4,5,5-tetramethyl-1,3,2-dioxaborolan-2-yl)pyrimidine (800 mg, 2.76 mmol) in water (2 mL) and isopropyl alcohol (10 mL) were added diacetoxypalladium (31 mg, 138 μmol), 2-Dicyclohexylphosphino-2′,6′-diisopropoxybiphenyl (129 mg, 276 μmol) and N-ethyl-N-isopropyl-propan-2-amine (713 mg, 5.5 mmol, 960 μL) at 25° C. under nitrogen atmosphere. The resulting mixture was stirred at 90° C. for 16 hours. Major product was observed by LCMS. The mixture was allowed to cool down to 25° C. The resulted mixture was concentrated under reduced pressure. The residue was purified by silica gel column chromatography, eluted with 0%-20% Methanol in Dichloromethane to afford 2-(4-cyclopropyl-6-methoxy-2-methylpyrimidin-5-yl)-5H-pyrrolo[3,2-d]pyrimidine (380 mg, 1.35 mmol, 49% yield) as an off-white solid. MS: m/z=282.05 [M+H]⁺. ¹H NMR (400 MHz, Chloroform-d) δ 9.33 (s, 1H), 7.86 (s, 1H), 6.90 (s, 1H), 3.93 (s, 3H), 2.59 (s, 3H), 1.80-1.74 (m, 1H), 1.26-1.22 (m, 2H), 0.89-0.85 (m, 2H).

### 1.2 (2-(4-cyclopropyl-6-methoxy-2-methylpyrimidin-5-yl)-5H-pyrrolo[3,2-d]pyrimidin-7-yl)(4-(1-methyl-4-(trifluoromethyl)-1H-imidazol-2-yl)phenyl)methanol

To a stirred solution of 2-(4-cyclopropyl-6-methoxy-2-methylpyrimidin-5-yl)-5H-pyrrolo[3,2-d]pyrimidine (130 mg, 462.12 μmol) and 4-[1-methyl-4-(trifluoromethyl)imidazol-2-yl]benzaldehyde (117 mg, 462 μmol) in water (5 mL) and isopropyl alcohol (5 mL) were added potassium carbonate (128 mg, 924 μmol) at 25° C. under nitrogen atmosphere. The resulting mixture was stirred at 50° C. for 16 hours. Major product was observed by LCMS. The resulted mixture was concentrated under reduced pressure. The residue was purified by RP-Flash chromatography with the following conditions: Column: C18 spherical, 20-30 um, 100A, 40 g; Mobile Phase A: 10 mM aq. NH₄HCO₃, Mobile Phase B: MeCN; Flow rate: 40 mL/min; Gradient: 5% B to 55% B in 15 min; 55% B to 55% B in 3 min; 55% B to 95% B in 5 min; Detector: UV 254 & 220 nm; RT: 16 min. The collected fractions were combined, concentrated and then lyophilized overnight to afford (2-(4-cyclopropyl-6-methoxy-2-methylpyrimidin-5-yl)-5H-pyrrolo[3,2-d]pyrimidin-7-yl)(4-(1-methyl-4-(trifluoromethyl)-1H-imidazol-2-yl)phenyl)methanol (25 mg, 46.68 μmol, 10% yield) as an off-white solid. MS: m/z=536.25 [M +H]⁺. ¹H NMR (400 MHz, Chloroform-d) δ 10.81 (s, 1H), 8.98 (s, 1H), 7.69-7.57 (m, 4H), 7.37 (s, 1H), 7.25 (s, 1H), 6.40 (s, 11H), 4.54 (s, 1H), 3.92 (s, 3H), 3.78 (s, 3H), 2.58 (s, 3H), 1.79-1.73 (m, 1H), 1.23-1.19 (m, 2H), 0.84-0.80 (m, 2H). ¹⁹F NMR (376 MHz, Chloroform-d) δ −62.54.

### 1.3 2-(4-cyclopropyl-6-methoxy-2-methylpyrimidin-5-yl)-7-(4-(1-methyl-4-(trifluoromethyl)-1H-imidazol-2-yl)benzyl)-5H-pyrrolo[3,2-d]pyrimidine

To a solution of (2-(4-cyclopropyl-6-methoxy-2-methylpyrimidin-5-yl)-5H-pyrrolo[3,2-d]pyrimidin-7-yl)(4-(1-methyl-4-(trifluoromethyl)-1H-imidazol-2-yl)phenyl)methanol (25 mg, 47 μmol) in chloroform (2 mL) were added trifluoroacetic acid (1 mL) and triethylsilane (2 mL) dropwise at 25° C. under nitrogen atmosphere. The resulting mixture was stirred at 25° C. for 16 hours under nitrogen atmosphere. Product could be detected by LCMS. The mixture was concentrated under reduced pressure. The residue was purified by RP-Flash with the following conditions: Column: C18 spherical, 20-30 um, 100A, 20 g; Mobile Phase A: Water (5 mM aq. NH₄HCO₃), Mobile Phase B: MeCN; Flow rate: 25 mL/min; Gradient: 5% B to 5% B in 5 min, 5% B to 40% B in 15 min, 40% B to 40% B in 3 min, 40% B to 95% B in 8 min; Detector: UV 254 & 210 nm; RT: 21 min. The collected fractions were combined, concentrated and then lyophilized overnight to afford 2-(4-cyclopropyl-6-methoxy-2-methylpyrimidin-5-yl)-7-(4-(1-methyl-4-(trifluoromethyl)-1H-imidazol-2-yl)benzyl)-5H-pyrrolo[3,2-d]pyrimidine (10.5 mg, 20.21 μmol, 43% yield) as an off-white solid. MS: m/z=520.25 [M+H]⁺. ¹H NMR (400 MHz, Chloroform-d) δ 9.23 (s, 1H), 7.55 (d, J=8.0 Hz, 2H), 7.49 (s, 1H), 7.43 (d, J=8.0 Hz, 2H), 7.34 (s, 1H), 4.29 (s, 2H), 3.94 (s, 3H), 3.77 (s, 3H), 2.58 (s, 3H), 1.84-1.77 (m, 1H), 1.26-1.22 (m, 2H), 0.84-0.80 (m, 2H). ¹⁹F NMR (376 MHz, Chloroform-d) δ −62.62.

### Example 101. 5-(4-cyclopropyl-6-methoxy-pyrimidin-5-yl)-3-[[4-[1-methyl-4-(trifluoromethyl)imidazol-2-yl]phenyl]methyl]-1-(2,2,2-trifluoroethyl)pyrazolo[4,3-d]pyrimidine (Compound 190) & 5-(4-cyclopropyl-6-methoxy-pyrimidin-5-yl)-3-[[4-[1-methyl-4-(trifluoromethyl)imidazol-2-yl]phenyl]methyl]-2-(2,2,2-trifluoroethyl)pyrazolo[4,3d]pyrimidine (Compound 131)

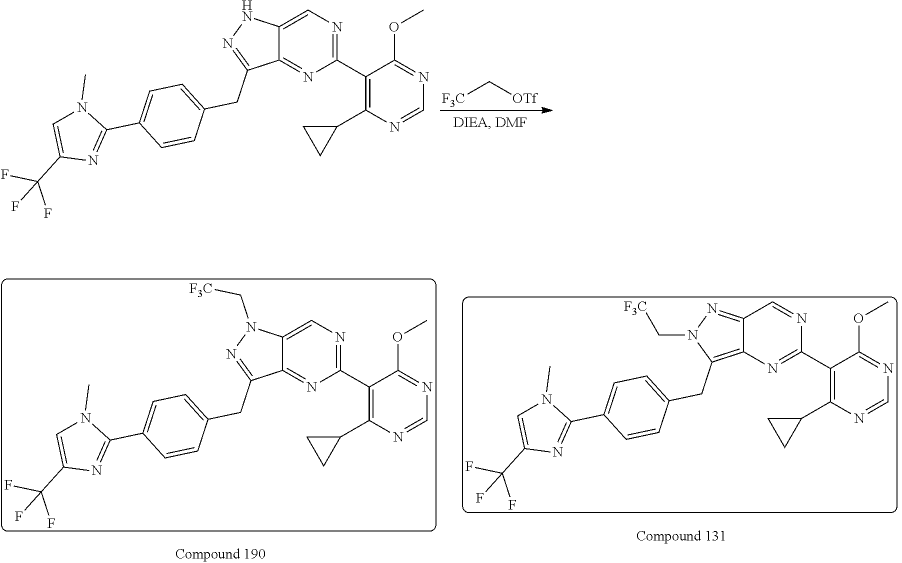

### 1.1 5-(4-cyclopropyl-6-methoxy-pyrimidin-5-yl)-3-[[4-[1-methyl-4-(trifluoromethyl)imidazol-2-yl]phenyl]methyl]-1-(2,2,2-trifluoroethyl)pyrazolo[4,3-d]pyrimidine (Compound 190) & 5-(4-cyclopropyl-6-methoxy-pyrimidin-5-yl)-3-[[4-[1-methyl-4-(trifluoromethyl)imidazol-2-yl]phenyl]methyl]-2-(2,2,2-trifluoroethyl)pyrazolo[4,3-d]pyrimidine (Compound 131)

To a solution of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (70 mg, 138.21 μmol) in N,N-dimethylformamide (0.8 mL) were added N,N-diisopropylethylamine (107 mg, 829 μmol) and 2,2,2-trifluoroethyl trifluoromethanesulfonate (128 mg, 553 μmol) at 0° C. The resulted mixture was stirred at room temperature for 20 hours. Major desired MS was detected on LCMS.

The combined reaction mixture which containing Compound 190 and Compound 131 (combined from two batches, theoretical 100% yield assumed, 157.96 μmol) was purified by reverse phase chromatography with the following conditions: Column: C18 gel column, 40 g, 20-35 μm; Mobile Phase A: 5 mM aq. NH₄HCO₃, Mobile Phase B: MeCN: Gradient: 0% B hold 5 min, up to 25% B within 5 min, 25% B to 70% B within 25 min, up to 95% B within 3 min, 95% B hold 5 min; Flow rate: 30 mL/min; RT₁=29.5 min, RT₂=30 min; Detector: UV 254 & 210 nm. The product-containing fractions (RT₂=30 min) were collected and evaporated to afford 5-(4-cyclopropyl-6-methoxy-pyrimidin-5-yl)-3-[[4-[1-methyl-4-(trifluoromethyl)imidazol-2-yl]phenyl]methyl]-1-(2,2,2-trifluoroethyl)pyrazolo[4,3-d]pyrimidine (Compound 190, 41.2 mg, 70.01 μmol, 44.32% yield) as a white solid. MS: m/z=589.20 [M+H]⁺. ¹H NMR (400 MHz, Chloroform-d): 9.26 (s, 1H), 8.71 (s, 1H), 7.59-7.54 (m, 4H), 7.31 (s, 1H), 5.07 (q, J=8.4 Hz, 2H), 4.53 (s, 2H), 3.95 (s, 3H), 3.75 (s, 3H), 1.75-1.62 (m, 1H), 1.30-1.24 (m, 2H), 0.94-0.86 (m, 2H). ¹⁹F NMR (376 MHz, Chloroform-d) δ −62.74, −70.72.

The product-containing fractions (RT₁=29.5 min) were collected and evaporated to afford 25 mg of Compound 131 (crude, containing ˜32% Compound 190) (RT₁=29.5 min) as a yellow solid. The crude product was purified by reverse phase chromatography with the following conditions: Column: C18 gel column, 20 g, 20-35 μm; Mobile Phase A: 5 mM aq. NH₄HCO₃, Mobile Phase B: MeCN: Gradient: 0% B hold 5 min, up to 30% B within 3 min, 30% B to 70% B within 25 min, up to 95% B within 3 min, 95% B hold 5 min; Flow rate: 25 mL/min; RT=26.5 min, Detector: UV 254 & 210 nm. The product-containing fractions were collected and evaporated. Then the product was further purified by Perp-HPLC with the following conditions: Column: XBridge Shield RP18 OBD Column, 30×150 mm, 5 μm; Mobile Phase A: 10 mM aq. NH₄HCO₃, Mobile Phase B: MeCN; Flow rate: 60 mL/min; Gradient: 45% B to 60% B in 8 min; Wave Length: 254 nm; RT: 6.4 min; The product-containing fractions were collected and evaporated in vacuo and then lyophilized overnight to give 5-(4-cyclopropyl-6-methoxy-pyrimidin-5-yl)-3-[[4-[1-methyl-4-(trifluoromethyl)imidazol-2-yl]phenyl]methyl]-2-(2,2,2-trifluoroethyl)pyrazolo[4,3-d]pyrimidine (Compound 131, 3.3 mg, 5.61 μmol, 3.55% yield, purified three times) as an off-white solid. MS: m/z=589.30 [M+H]⁺. ¹H NMR (400 MHz, Chloroform-d) δ 9.63 (s, 1H), 8.70 (s, 1H), 7.64-7.59 (m, 2H), 7.35-7.30 (m, 3H), 4.92 (q, J=8.0 Hz, 2H), 4.71 (s, 2H), 3.98 (s, 3H), 3.77 (s, 3H), 1.82-1.75 (m, 1H), 1.34-1.23 (m, 2H), 0.98-0.90 (m, 2H). ¹⁹F NMR (376 MHz, Chloroform-d) δ −62.80, −69.15.

### Example 102. 2-(4-cyclopropyl-6-methoxy-pyrimidin-5-yl)-7-[[4-[1-methyl-4-(trifluoromethyl)imidazol-2-yl]phenyl]methyl]-5-(2,2,2-trifluoroethyl)pyrrolo[3,2-d]pyrimidine Compound 125

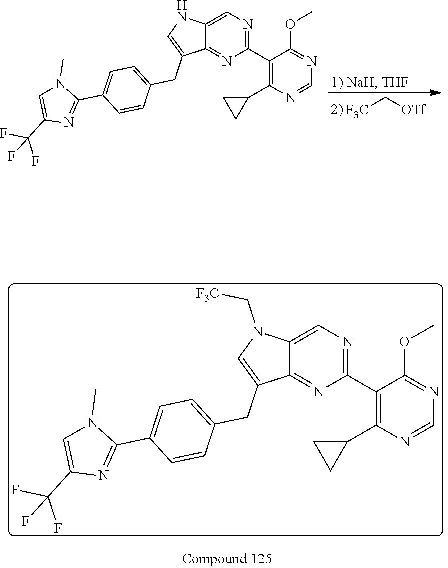 [SMILES: COc1ncnc(C2CC2)c1-c1ncc2[nH]cc(Cc3ccc(-c4nc(C(F)(F)F)cn4C)cc3)c2n1 ; O=S(=O)(OCC(F)(F)F)C(F)(F)F ; COc1ncnc(C2CC2)c1-c1ncc2c(n1)c(Cc1ccc(-c3nc(C(F)(F)F)cn3C)cc1)cn2CC(F)(F)F]

### 1.1 2-(4-cyclopropyl-6-methoxy-pyrimidin-5-yl)-7-[[4-[1-methyl-4-(trifluoromethyl)imidazol-2-yl]phenyl]methyl]-5-(2,2,2-trifluoroethyl)pyrrolo[3,2-d]pyrimidine

To a stirred solution of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (30 mg, 59.35 μmol) in tetrahydrofuran (1 mL) was added sodium hydride (3.6 mg, 89 μmol, 60%) in three portions at 0° C. under nitrogen atmosphere. After stirred at 0° C. for 0.5 hour, 2,2,2-trifluoroethyl trifluoromethanesulfonate (28 mg, 119 μmol, 17 μL) was added with stirring at 25° C. The mixture was stirred at 25° C. for 4 hours. The resulting solution was detected by LCMS, major desired product. The resulting solution was quenched by the addition of saturated ammonium chloride aqueous solution (3 mL) at 25° C. The resulting mixture was extracted with ethyl acetate (3×5 mL). The combined organic layers were dried over anhydrous sodium sulfate. After filtration, the filtrate was concentrated under reduced pressure. The residue was purified by Prep-TLC, eluted with 6% methanol in dichloromethane to give 30 mg crude product. The resulted mixture was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 53% B in 13 min, 53% B to 53% B in 2 min, 53% B to 95% B in 7 min, 95% B to 95% B in 3 min; Detector: UV 254 & 220 nm: RT: 19 min. The collected fractions were combined, concentrated and then lyophilized to give 2-(4-cyclopropyl-6-methoxy-pyrimidin-5-yl)-7-[[4-[1-methyl-4-(trifluoromethyl)imidazol-2-yl]phenyl]methyl]-5-(2,2,2-trifluoroethyl)pyrrolo[3,2-d]pyrimidine (19.6 mg, 33.36 μmol, 56% yield) as an off-white solid. MS: m/z=588.35 [M+H]⁺. ¹H NMR (400 MHz, Chloroform-d) δ 9.04 (s, 1H), 8.74 (s, 1H), 7.61 (d, J=8.4 Hz, 2H), 7.48 (d, J=8.0 Hz, 2H), 7.34 (s, 1H), 7.18 (s, 1H), 4.77-4.71 (m, 2H), 4.29 (s, 2H), 3.99 (s, 3H), 3.79 (s, 3H), 1.79-1.71 (m, 1H), 1.39-1.32 (m, 2H), 0.99-0.92 (m, 2H). ¹⁹F NMR (376 MHz, Chloroform-d) δ −62.74, 71.39.

### Example 103. 5-(4-cyclopropyl-6-methoxy-pyrimidin-5-yl)-1-isopropyl-3-[[4-[1-methyl-4-(trifluoromethyl)imidazol-2-yl]phenyl]methyl]pyrazolo[4,3-d]pyrimidine (Compound 98) & 5-(4-cyclopropyl-6-methoxy-pyrimidin-5-yl)-2-isopropyl-3-[[4-[1-methyl-4-(trifluoromethyl)imidazol-2-yl]phenyl]methyl]pyrazolo[4,3-d]pyrimidine (Compound 104)

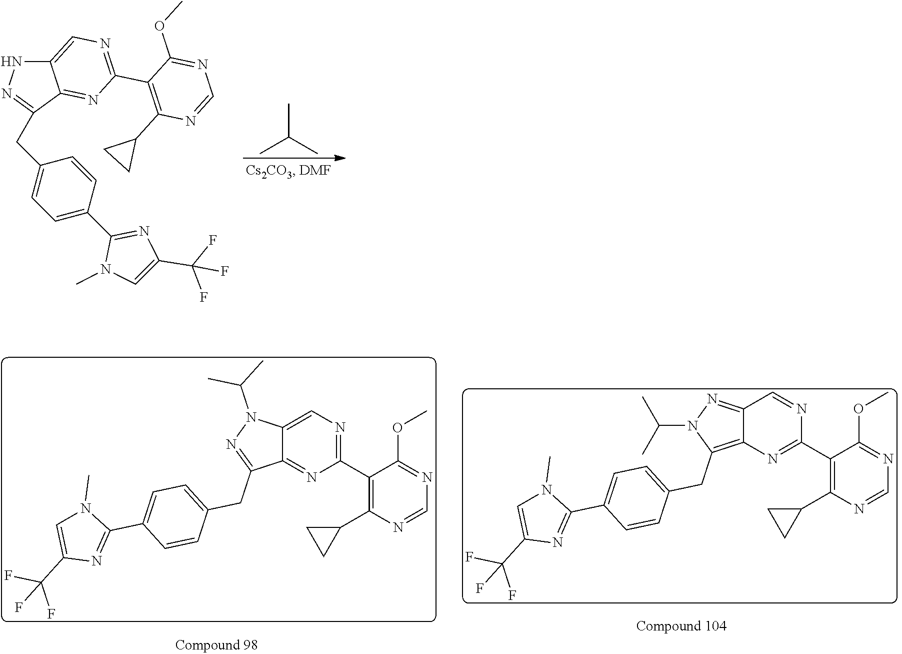

1.1 5-(4-cyclopropyl-6-methoxy-pyrimidin-5-yl)-1-isopropyl-3-[[4-[1-methyl-4-(trifluoromethyl)imidazol-2-yl]phenyl]methyl]pyrazolo[4,3-d]pyrimidine & 5-(4-cyclopropyl-6-methoxy-pyrimidin-5-yl)-2-isopropyl-3-[[4-[1-methyl-4-(trifluoromethyl)imidazol-2-yl]phenyl]methyl]pyrazolo[4,3-d]pyrimidine

To a solution of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (50 mg, 98.72 μmol) and cesium carbonate (64.33 mg, 197.44 μmol) in N,N-dimethylformamide (1 mL) was added 2-iodopropane (34 mg, 197 μmol, 19.7 μL) dropwise at 0° C., and then stirred at 110° C. for 2 hours. The reaction progress was monitored by LCMS. The resulted mixture was cooled down to room temperature, diluted with 40 mL of ethyl acetate, washed with brine (3×10 mL). The organic layer was dried over anhydrous sodium sulfate and filtered. The filtrate was concentrated under reduced pressure. The residue was purified by Prep-TLC, eluted with 9% methanol in dichloromethane to give a crude product. The crude product was purified by RP-Flash chromatography with the following conditions: Column: C18 spherical, 20-30 um, 100A, 40 g; Mobile Phase A: Water (10 mM aq. NH₄HCO₃), Mobile Phase B: MeCN; Flow rate: 35 mL/min; Gradient: 2% B to 2% B in 5 min, 2% B to 38% B in 21 min; 38% B to 38% B in 6.2 min; 38% B to 98% B in 5 min; 98% B to 98% B in 5 min; Detector: UV 254 & 210 nm: RT1: 27 min; RT2: 29 min.

The first eluting peak (RT1: 27 min) were combined, concentrated and then lyophilized to give 5-(4-cyclopropyl-6-methoxy-pyrimidin-5-yl)-1-isopropyl-3-[[4-[1-methyl-4-(trifluoromethyl)imidazol-2-yl]phenyl]methyl]pyrazolo[4,3-d]pyrimidine (Compound 98) (18.5 mg, 33.72 μmol, 34% yield) as an off-white solid. MS: m/z=549.40 [M+H]f. ¹H NMR (400 MHz, Chloroform-d) δ 9.26 (s, 1H), 8.69 (s, 1H), 7.59-7.52 (m, 4H), 7.29 (s, 1H), 5.02-4.92 (m, 1H), 4.53 (s, 2H), 3.94 (s, 3H), 3.74 (s, 3H), 1.85-1.68 (m, 7H), 1.28-1.19 (m, 2H), 0.95-0.83 (m, 2H). ¹⁹F NMR (377 MHz, Chloroform-d) δ −62.75.

The second eluting peak (RT2: 29 min) were combined, concentrated and then lyophilized to give 5-(4-cyclopropyl-6-methoxy-pyrimidin-5-yl)-2-isopropyl-3-[[4-[1-methyl-4-(trifluoromethyl)imidazol-2-yl]phenyl]methyl]pyrazolo[4,3-d]pyrimidine (Compound 104) (6.4 mg, 11.67 μmol, 11% yield) as an off-white solid. MS: m/z=549.40 [M+H]⁺. ¹H NMR (400 MHz, Chloroform-d) δ 9.58 (s, 1H), 8.69 (s, 1H), 7.58 (d, J=7.6 Hz, 2H), 7.34-7.28 (m, 3H), 4.88-4.82 (m, 1H), 4.65 (s, 2H), 3.97 (s, 3H), 3.74 (s, 3H), 1.89-1.75 (m, 1H), 1.54 (d, J=6.4 Hz, 6H), 1.32-1.21 (m, 2H), 0.96-0.85 (m, 2H). ¹⁹F NMR (377 MHz, Chloroform-d) δ −62.77.

### Example 104. 5-(4-cyclopropyl-6-methoxy-pyrimidin-5-yl)-3-[[3,5-difluoro-4-[1-methyl-4-(trifluoromethyl)imidazol-2-yl]phenyl]methyl]-1H-pyrazolo[4,3-d]pyrimidine Compound 171

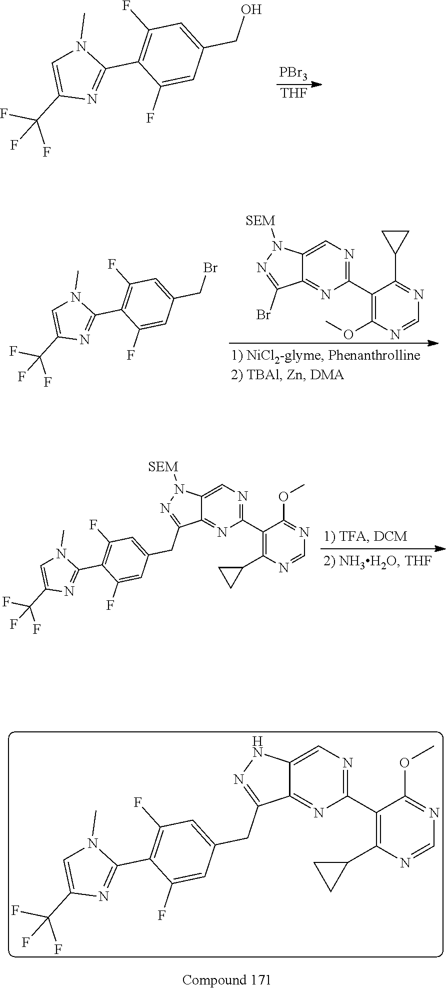

### 1.1 2-[4-(bromomethyl)-2,6-difluoro-phenyl]-1-methyl-4-(trifluoromethyl)imidazole

To a stirred solution of [3,5-difluoro-4-[1-methyl-4-(trifluoromethyl)imidazol-2-yl]phenyl]methanol (150 mg, 513.34 μmol) in THF (1.5 mL) was added tribromophosphane (417 mg, 1.54 mmol, 145 μL) dropwise over 5 min at 0° C. under nitrogen atmosphere. The resulting mixture was stirred for 2 hours at 25° C. under nitrogen atmosphere then quenched by the addition of saturated sodium bicarbonate aqueous solution (10 mL) at 0° C. The resulting mixture was concentrated under reduced pressure. The residue was purified by Prep-TLC (30% ethyl acetate in petroleum ether) to afford 2-[4-(bromomethyl)-2,6-difluoro-phenyl]-1-methyl-4-(trifluoromethyl)imidazole (100 mg, 281.61 μmol, 55% yield) as a yellow solid. MS: m/z=354.90, 356.90 [M+H]⁻.

### 1.2 2-[[5-(4-cyclopropyl-6-methoxy-pyrimidin-5-yl)-3-[[3,5-difluoro-4-[1-methyl-4-(trifluoromethyl)imidazol-2-yl]phenyl]methyl]pyrazolo[4,3-d]pyrimidin-1-yl]methoxy]ethyl-trimethyl-silane

A mixture of Nickel (II) chloride dimethoxyethane adduct (25 mg, 113 μmol) and 1,10-phenanthroline (20.3 mg, 113 μmol) in N,N-dimethylacetamide (1 mL) was stirred for 10 min at 25° C. under nitrogen atmosphere. Then was added 2-[4-(bromomethyl)-2,6-difluoro-phenyl]-1-methyl-4-(trifluoromethyl)imidazole (100 mg, 281.61 μmol), tetrabutylammonium iodide (101 mg, 273 μmol) and 2-[[3-bromo-5-(4-cyclopropyl-6-methoxy-pyrimidin-5-yl)pyrazolo[4,3-d]pyrimidin-1-yl]methoxy]ethyl-trimethyl-silane (134 mg, 282 μmol) in N,N-dimethylacetamide (1 mL) and then was added zinc powder (37 mg, 563 μmol) at 25° C. under nitrogen atmosphere. The resulting mixture was stirred at 50° C. for 16 hours under nitrogen atmosphere. The resulting mixture was diluted with ethyl acetate (20 mL). The resulting mixture were washed with brine (10 mL), dried over anhydrous sodium sulfate. After filtration, the filtrate was concentrated under reduced pressure. The residue was purified by Prep-TLC (50% ethyl acetate in petroleum ether) to afford 2-[[5-(4-cyclopropyl-6-methoxy-pyrimidin-5-yl)-3-[[3,5-difluoro-4-[1-methyl-4-(trifluoromethyl)imidazol-2-yl]phenyl]methyl]pyrazolo[4,3-d]pyrimidin-1-yl]methoxy]ethyl-trimethyl-silane (20 mg, 29.73 μmol, 10% yield) as a yellow solid. MS: m/z=673.25 [M+H]⁺.

### 1.3 5-(4-cyclopropyl-6-methoxy-pyrimidin-5-yl)-3-[[3,5-difluoro-4-[1-methyl-4-(trifluoromethyl)imidazol-2-yl]phenyl]methyl]-1H-pyrazolo[4,3-d]pyrimidine

To a stirred solution of 2-[[5-(4-cyclopropyl-6-methoxy-pyrimidin-5-yl)-3-[[3,5-difluoro-4-[1-methyl-4-(trifluoromethyl)imidazol-2-yl]phenyl]methyl]pyrazolo[4,3-d]pyrimidin-1-yl]methoxy]ethyl-trimethyl-silane (13 mg, 19.32 μmol) in dichloromethane (1 mL) was added 2,2,2-trifluoroacetic acid (1.48 g, 12.98 mmol, 1 mL) at 25° C. The resulting mixture was stirred for 1 hour at 25° C. then concentrated under reduced pressure. The residue was dissolved in THF (1 mL) and ammonium hydroxide (1 mL, 28% aqueous solution) at 25° C. The resulting mixture was stirred for 1 hour at 25° C. then concentrated under reduced pressure. The residue was purified by Prep-TLC, eluted with 50% ethyl acetate in petroleum ether to afford crude product. The obtained crude product was further purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 95% B in 25 min; Detector: UV 254 & 210 nm; RT: 17 min. The collected fractions were combined, concentrated under reduced pressure and lyophilized to give 5-(4-cyclopropyl-6-methoxy-pyrimidin-5-yl)-3-[[3,5-difluoro-4-[1-methyl-4-(trifluoromethyl)imidazol-2-yl]phenyl]methyl]-1H-pyrazolo[4,3-d]pyrimidine (3.1 mg, 5.71 μmol, 29% yield) as an off-white solid. MS: m/z=543.15 [M+H]⁺. ¹H NMR (400 MHz, Chloroform-d) δ 12.79 (br., 1H), 9.23 (s, 1H), 8.69 (s, 1H), 7.48 (s, 1H), 7.15 (d, J=8.8 Hz, 2H), 4.48 (s, 2H), 3.94 (s, 3H), 3.67 (s, 3H), 1.78-1.67 (m, 1H), 1.31-1.23 (m, 2H), 0.98-0.85 (m, 2H). ¹⁹F NMR (376 MHz, Chloroform-d) δ −62.37, −110.31.

### Example 105. 5-(4-cyclopropyl-6-methoxy-2-methylpyrimidin-5-yl)-3-(4-(1-methyl-4-(trifluoromethyl)-1H-imidazol-2-yl)benzyl)-1H-pyrazolo[4,3-d]pyrimidine Compound 184

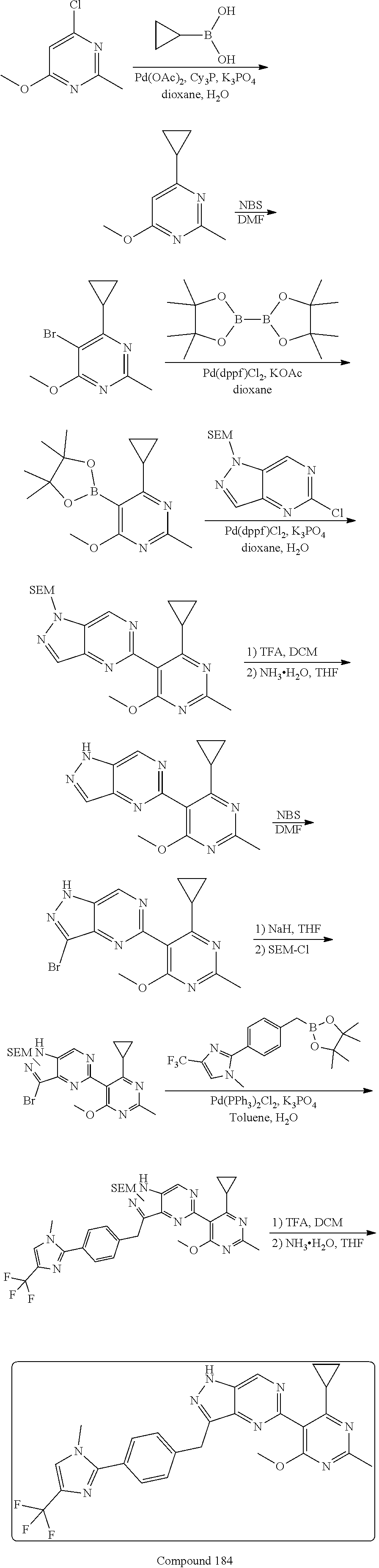

### 1.1 4-cyclopropyl-6-methoxy-2-methylpyrimidine

To a mixture of 4-chloro-6-methoxy-2-methylpyrimidine (4.5 g, 28.38 mmol) and cyclopropylboronic acid (3.66 g, 42.56 mmol) in water (6 mL) and 1,4-dioxane (30 mL) were added palladium (II) acetate (637 mg, 2.84 mmol), tricyclohexyl phosphine (796 mg, 2.84 mmol) and potassium phosphate tribasic (12.05 g, 56.75 mmol) at 25° C. under nitrogen atmosphere. The reaction mixture was stirred at 100° C. for 16 hours. The mixture was allowed to cool down to 25 TC. The residue was purified by silica gel column chromatography, eluted with 0%-40% ethyl acetate in petroleum ether to afford 4-cyclopropyl-6-methoxy-2-methylpyrimidine (4 g, 24.36 mmol, 86% yield) as a yellow solid. MS: m/z=165.10 [M+H]⁺. ¹H NMR (400 MHz, Chloroform-d) δ 6.30 (s, 1H), 3.93 (s, 3H), 2.54 (s, 3H)1.96-1.85 (m, 1H), 1.09-0.92 (m, 4H).

### 1.2 5-bromo-4-cyclopropyl-6-methoxy-2-methylpyrimidine

To the solution of 4-cyclopropyl-6-methoxy-2-methylpyrimidine (3.5 g, 21.31 mmol) in N,N-Dimethylformamide (35 mL) was added N-Bromosuccinimide (5.69 g, 31.97 mmol) at 25° C. under nitrogen atmosphere. The resulting mixture was stirred at 25° C. for 16 hours under nitrogen atmosphere. Product could be detected by LCMS. The reaction was quenched with brine (200 mL), and then extracted with ethyl acetate (3×200 mL). The combined organic layers were dried over anhydrous sodium sulfate and filtered. The filtrate was concentrated under reduced pressure. The residue was purified by silica gel column chromatography, eluted with 0%-40% ethyl acetate in petroleum ether to afford 5-bromo-4-cyclopropyl-6-methoxy-2-methylpyrimidine (3.5 g, 14.40 mmol, 68% yield) as a yellow solid. MS: m/z=243.00, 244.95 [M+H]⁺. ¹H NMR (400 MHz, Chloroform-d) δ 4.04 (s, 3H), 2.52 (s, 3H), 2.51-2.44 (m, 1H), 1.26-1.18 (m, 2H), 1.12-1.04 (m, 2H).

### 1.3 4-cyclopropyl-6-methoxy-2-methyl-5-(4,4,5,5-tetramethyl-1,3,2-dioxaborolan-2-yl)pyrimidine

To a mixture of 5-bromo-4-cyclopropyl-6-methoxy-2-methylpyrimidine (I g, 4.11 mmol) and Bis(pinacolato)diboron (1.57 g, 6.17 mmol) in 1,4-dioxane (9.5 mL) were added 1,1′-Bis(diphenylphosphino)ferrocene-palladium(II)dichloride (336 mg, 411 μmol) and Potassium Acetate (1.21 g, 12.34 mmol) at 25° C. under nitrogen atmosphere. The resulting mixture was stirred at 100° C. for 16 hours. Product could be detected by LCMS. The mixture was allowed to cool down to 25° C. The mixture was filtered. The filtrate was concentrated under reduced pressure. The residue was purified by silica gel column chromatography, eluted with 0%-70% ethyl acetate in petroleum ether to afford 4-cyclopropyl-6-methoxy-2-methyl-5-(4,4,5,5-tetramethyl-1,3,2-dioxaborolan-2-yl)pyrimidine (520 mg, 1.79 mmol, 44% yield) as an off-white solid. MS: m/z=291.10 [M+H]⁺. ¹H NMR (400 MHz, Chloroform-d) δ 3.92 (s, 3H), 2.48 (s, 3H), 2.16-2.06 (m, 1H), 1.40 (s, 12H), 1.21-1.13 (m, 2H), 1.00-0.88 (m, 2H).

### 1.4 5-(4-cyclopropyl-6-methoxy-2-methylpyrimidin-5-yl)-1-((2-(trimethylsilyl)ethoxy)methyl)-1H-pyrazolo[4,3-d]pyrimidine

To a mixture of 4-cyclopropyl-6-methoxy-2-methyl-5-(4,4,5,5-tetramethyl-1,3,2-dioxaborolan-2-yl)pyrimidine (520 mg, 1.79 mmol) and 5-chloro-1-((2-(trimethylsilyl)ethoxy)methyl)-1H-pyrazolo[4,3-d]pyrimidine (510 mg, 1.79 mmol) in water (1 mL) and 1,4-dioxane (5 mL) were added Potassium phosphate tribasic (761 mg, 3.58 mmol) and 1,1′-Bis(diphenylphosphino)ferrocene-palladium(II)dichloride (146 mg, 179 μmol) at 25° C. under nitrogen atmosphere. The resulting mixture was stirred at 90° C. for 16 hours. The mixture was allowed to cool down to 25° C. The mixture was filtered. The filtrate was concentrated under reduced pressure. The residue was purified by silica gel column chromatography, eluted with 0%-70% ethyl acetate in petroleum ether to afford 5-(4-cyclopropyl-6-methoxy-2-methylpyrimidin-5-yl)-1-((2-(trimethylsilyl)ethoxy)methyl)-1H-pyrazolo[4,3-d]pyrimidine (550 mg, 1.33 mmol, 74% yield) as an off-white solid. MS: m/z=413.15 [M+H]⁺. 1.5 5-(4-cyclopropyl-6-methoxy-2-methylpyrimidin-5-yl)-1H-pyrazolo[4,3-d]yrimidine

To the solution of 2-[[5-(4-cyclopropyl-6-methoxy-2-methyl-pyrimidin-5-yl)pyrazolo[4,3-d]pyrimidin-1-yl]methoxy]ethyl-trimethyl-silane (550 mg, 1.33 mmol) in dichloromethane (4 mL) was added Trifluoroacetic acid (4 mL) at 0° C. under nitrogen atmosphere. The reaction mixture was stirred at 25° C. for 1 hour and then concentrated under reduced pressure. The residue was dissolved in Tetrahydrofuran (4 mL) and Ammonium hydroxide (4 mL). The reaction mixture was stirred at 25° C. for 1 hour. The product can be found by LCMS. The mixture was concentrated under reduced pressure. The residue was purified by silica gel column chromatography, eluted with 1%-15% methanol in dichloromethane to afford 5-(4-cyclopropyl-6-methoxy-2-methylpyrimidin-5-yl)-1H-pyrazolo[4,3-d]pyrimidine (280 mg, 991.85 μmol, 74% yield) as yellow solid. MS: m/z=283.05 [M+H]⁺.

### 1.6 3-bromo-5-(4-cyclopropyl-6-methoxy-2-methylpyrimidin-5-yl)-1H-pyrazolo[4,3-d]pyrimidine

To a solution of 5-(4-cyclopropyl-6-methoxy-2-methylpyrimidin-5-yl)-1H-pyrazolo[4,3-d]pyrimidine (280 mg, 991.85 μmol) in N,N-Dimethylformamide (3 mL) was added N-Bromosuccinimide (211.84 mg, 1.19 mmol) at 25° C. under nitrogen atmosphere. The mixture was stirred at 25° C. for 16 hours. The resulted solution was detected by LCMS, major desired product. The mixture was diluted with water (50 mL), extracted with ethyl acetate (3-30 mL). The combined organic phase was washed with brine (50 mL), dried over anhydrous sodium sulfate. After filtration, the filtrate was concentrated under reduced pressure. The residue was purified by silica gel column chromatography, eluted with 0%-15% methanol in dichloromethane to afford 3-bromo-5-(4-cyclopropyl-6-methoxy-2-methylpyrimidin-5-yl)-1H-pyrazolo[4,3-d]pyrimidine (260 mg, 719.83 μmol, 73% yield) as a yellow solid. MS: m/z=360.95, 362.95 [M+H]⁺. ¹H NMR (400 MHz, Chloroform-d) δ 11.07 (s, 1H), 9.36 (s, 1H), 3.93 (s, 3H), 2.67 (s, 3H), 1.72-1.64 (s, 1H), 1.36-1.28 (m, 2H), 0.96-0.88 (m, 2H).

### 1.7 3-bromo-5-(4-cyclopropyl-6-methoxy-2-methylpyrimidin-5-yl)-1-((2-(trimethylsilyl)ethoxy)methyl)-1H-pyrazolo[4,3-d]pyrimidine & 3-bromo-5-(4-cyclopropyl-6-methoxy-2-methylpyrimidin-5-yl)-2-((2-(trimethylsilyl)ethoxy)methyl)-2H-pyrazolo[4,3-d]pyrimidine

To a solution of 3-bromo-5-(4-cyclopropyl-6-methoxy-2-methylpyrimidin-5-yl)-1H-pyrazolo[4,3-d]pyrimidine (260 mg, 720 μmol) in Tetrahydrofuran (3 mL) was added Sodium hydride (35 mg, 864 μmol, 60% purity) at 0° C., then was stirred at 0° C. for 1 hour. The resulting solution was added 2-(Trimethylsilyl)ethoxymethyl chloride (144 mg, 864 μmol) at 25° C. The above solution was stirred at 25° C. for 2 hours. The resulted solution was detected by LCMS, major desired product. The resulted solution was quenched by saturated ammonium chloride aqueous solution (50 mL), extracted by ethyl acetate (30 mL×3). The combined organic phase was washed with brine (50 mL), dried over anhydrous sodium sulfate. After filtration, the filtrate was concentrated under reduced pressure The residue was purified by silica gel column chromatography, eluted with 0-5% methanol in dichloromethane to afford 3-bromo-5-(4-cyclopropyl-6-methoxy-2-methylpyrimidin-5-yl)-1-((2-(trimethylsilyl)ethoxy)methyl)-1H-pyrazolo[4,3-d]pyrimidine & 3-bromo-5-(4-cyclopropyl-6-methoxy-2-methylpyrimidin-5-yl)-2-((2-(trimethylsilyl)ethoxy)methyl)-2H-pyrazolo[4,3-d]pyrimidine (240 mg, 488.35 μmol, 68% yield) as yellow solid. MS: m/z=491.10, 493.05 [M+H]⁺.

### 1.8 5-(4-cyclopropyl-6-methoxy-2-methylpyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-cyclopropyl-6-methoxy-2-methylpyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine

To a solution of 3-bromo-5-(4-cyclopropyl-6-methoxy-2-methylpyrimidin-5-yl)-1-((2-(trimethylsilyl)ethoxy)methyl)-1H-pyrazolo[4,3-d]pyrimidine & 3-bromo-5-(4-cyclopropyl-6-methoxy-2-methylpyrimidin-5-yl)-2-((2-(trimethylsilyl)ethoxy)methyl)-2H-pyrazolo[4,3-d]pyrimidine (240 mg, 488.35 μmol) in water (0.5 mL) and Toluene (2.5 mL) were added 1-methyl-2-(4-((4,4,5,5-tetramethyl-1,3,2-dioxaborolan-2-yl)methyl)phenyl)-4-(trifluoromethyl)-1H-imidazole (179 mg, 488 μmol), Bis(triphenylphosphine)palladium(II) chloride (101 mg, 146.5 μmol) and Potassium phosphate tribasic (207 mg, 977 μmol) at 25° C. The mixture was stirred at 90° C. for 16 hours. The mixture was cooled down to room temperature. The mixture was filtered and concentrated under reduced pressure. The residue was purified by silica gel column chromatography, eluted with 0-80% ethyl acetate in petroleum ether to afford 5-(4-cyclopropyl-6-methoxy-2-methylpyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-cyclopropyl-6-methoxy-2-methylpyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (90 mg, 138 μmol, 29% yield) as a yellow solid. MS: m/z=651.30 [M+H]⁺. ¹H NMR (400 MHz. Chloroform-d) δ 9.34 (s, 1H), 7.58-7.52 (m, 4H), 7.31 (s, 1H), 5.82 (s, 2H), 4.52 (s, 2H), 3.96 (s, 3H), 3.75 (s, 3H), 3.68-3.62 (m, 2H) 2.70 (s, 3H), 1.69-1.57 (m, 1H), 1.31-1.23 (m, 2H), 1.01-0.88 (m, 4H), 0.02 (s, 9H).

### 1.9 5-(4-cyclopropyl-6-methoxy-2-methylpyrimidin-5-yl)-3-(4-(1-methyl-4-(trifluoromethyl)-1H-imidazol-2-yl)benzyl)-1H-pyrazolo[4,3-d]pyrimidine

To the solution of 5-(4-cyclopropyl-6-methoxy-2-methylpyrimidin-5-yl)-3-(4-(1-methyl-4-(trifluoromethyl)-1H-imidazol-2-yl)benzyl)-1-((2-(trimethylsilyl)ethoxy)methyl)-1H-pyrazolo[4,3-d]pyrimidine & 5-(4-cyclopropyl-6-methoxy-2-methylpyrimidin-5-yl)-3-(4-(1-methyl-4-(trifluoromethyl)-1H-imidazol-2-yl)benzyl)-2-((2-(trimethylsilyl)ethoxy)methyl)-2H-pyrazolo[4,3-d]pyrimidine (100 mg, 153.66 μmol) in dichloromethane (1 mL) was added Trifluoroacetic acid (1 mL) at 0° C. under nitrogen atmosphere. The reaction mixture was stirred at 25° C. for 1 hour and then concentrated under reduced pressure. The residue was dissolved in Tetrahydrofuran (1 mL) and Ammonium hydroxide (1 mL). The reaction mixture was stirred at 25° C. for 1 hour. The product can be found by LCMS. The mixture was concentrated under reduced pressure. The residue was continued purified by Prep-TLC to give a crude product. The residue was purified by RP-Flash chromatography with the following conditions: Column: C18 spherical, 20-35 um, 100A, 20 g; Mobile Phase A: 10 mM aq. NH₄HCO₃, Mobile Phase B: MeCN; Flow rate: 30 mL/min; Gradient: 5% B to 5% B in 5 min, 5% B to 45% B in 15 min; 45% B to 45% B in 3 min; 45% B to 95% B in 5 min; Detector: UV 254 & 220 nm: RT: 21 min. The collected fractions were combined and concentrated under reduced pressure and then lyophilized to afford 5-(4-cyclopropyl-6-methoxy-2-methylpyrimidin-5-yl)-3-(4-(1-methyl-4-(trifluoromethyl)-1H-imidazol-2-yl)benzyl)-1H-pyrazolo[4,3-d]pyrimidine (37.4 mg, 71.85 μmol, 47% yield) as an off-white solid. MS: m/z=521.25 [M+H]⁺. ¹H NMR (400 MHz, Chloroform-d) δ 9.18 (s, 1H), 7.66 (d, J=8.0 Hz, 2H), 7.59 (d. J=8.0 Hz, 2H), 7.39 (s, 1H), 4.54 (s, 2H), 3.91 (s, 3H), 3.82 (s, 3H), 2.65 (s, 3H), 1.70-1.60 (m, 1H), 1.34-1.23 (s, 2H), 0.85-0.74 (s, 2H). ¹⁹F NMR (377 MHz, Chloroform-d) δ −62.26.

### Example 106. 1-[2-(2-cyclopropyl-3-pyridyl)-7-[[4-[1-methyl-4-(trifluoromethyl)imidazol-2-yl]phenyl]methyl]-5-pyrrolo[3,2-d]pyrimidin-6-yl]-N-methyl-methanamine Compound 178

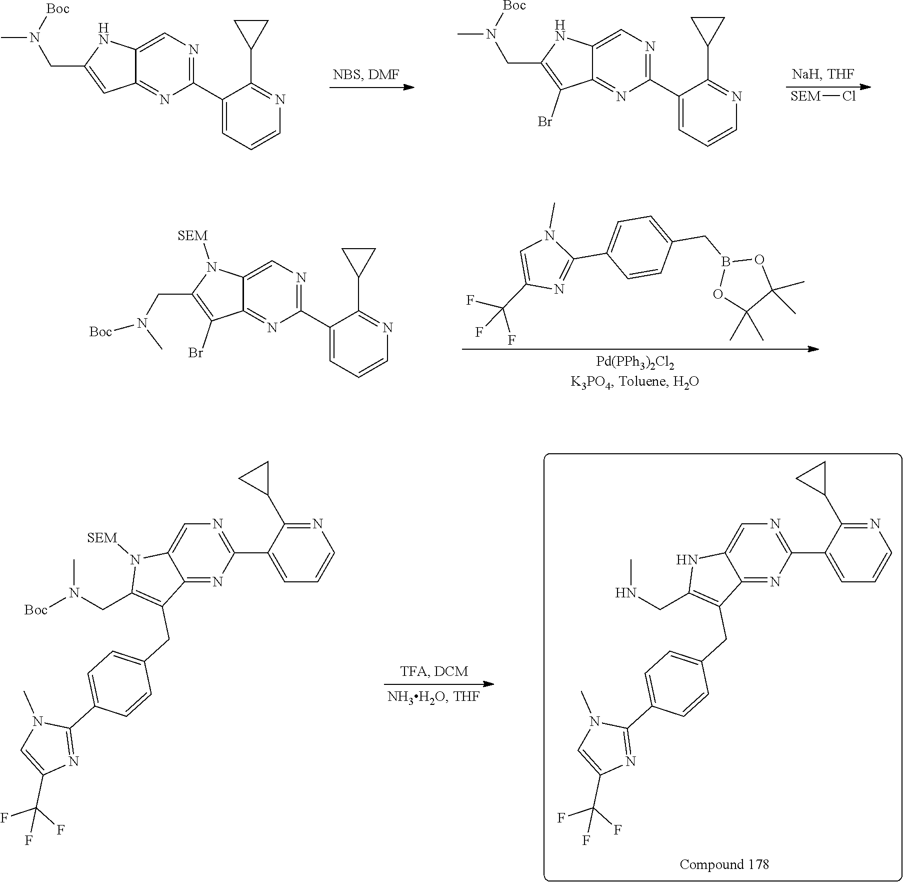

### 1.1 tert-butyl N-[[7-bromo-2-(2-cyclopropyl-3-pyridyl)-5H-pyrrolo[3,2-d]pyrimidin-6-yl]methyl]-N-methyl-carbamate

To a stirred solution of tert-butyl N-[[2-(2-cyclopropyl-3-pyridyl)-5H-pyrrolo[3,2-d]pyrimidin-6-yl]methyl]-N-methyl-carbamate (160 mg, 422 μmol) in N,N-dimethylformamide (3 mL) was added N-Bromosuccinimide (75 mg, 422 μmol) under nitrogen atmosphere at 25° C. The resulting mixture was stirred for 2 hours at 25° C. The reaction was quenched by the addition of saturated sodium hydrogen carbonate aqueous solution (100 mL). The resulted mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 45%-50% ethyl acetate in petroleum ether to give tert-butyl N-[[7-bromo-2-(2-cyclopropyl-3-pyridyl)-5H-pyrrolo[3,2-d]pyrimidin-6-yl]methyl]-N-methyl-carbamate (160 mg, 349.08 μmol, 83% yield) as a yellow oil. MS: m/z=458.20, 450.20 [M+H]⁺.

### 1.2 tert-butyl N-[[7-bromo-2-(2-cyclopropyl-3-pyridyl)-5-(2-trimethylsilylethoxymethyl)pyrrolo[3,2-d]pyrimidin-6-yl]methyl]-N-methyl-carbamate

To a stirred solution of tert-butyl N-[[7-bromo-2-(2-cyclopropyl-3-pyridyl)-5H-pyrrolo[3,2-d]pyrimidin-6-yl]methyl]-N-methyl-carbamate (160 mg, 349.08 μmol) in tetrahydrofuran (3 mL) was added sodium hydride (17 mg, 419 μmol, 60% purity) under nitrogen atmosphere at 0° C. The resulting mixture was stirred for 1 hour at 0° C. To the above mixture was added (2-(chloromethoxy)ethyl)trimethylsilane (70 mg, 419 μmol) under nitrogen atmosphere at 0° C. The resulted mixture was stirred at 25° C. for 2 hours. The reaction was quenched by the addition of saturated ammonium chloride aqueous solution (100 mL). The resulting mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 32%-33% ethyl acetate in petroleum ether to give a mixture of tert-butyl N-[[7-bromo-2-(2-cyclopropyl-3-pyridyl)-5-(2-trimethylsilylethoxymethyl)pyrrolo[3,2-d]pyrimidin-6-yl]methyl]-N-methyl-carbamate (150 mg, 254.84 μmol, 73% yield) as a yellow oil. MS: m/z=590.40, 592.40 [M+H]⁺. ¹H NMR (400 MHz, Chloroform-d) δ 9.12 (s, 1H), 8.66 (s, 1H), 8.41 (s, 1H), 7.38 (s, 1H), 5.73 (s, 2H), 4.93 (s, 2H), 3.58-3.54 (m, 2H), 2.86 (s, 3H), 1.73-1.61 (m, 1H), 1.55 (s, 9H), 1.31-1.25 (m, 2H), 1.01-0.89 (m, 4H), −0.01 (s, 9H).

### 1.3 tert-butyl N-[[2-(2-cyclopropyl-3-pyridyl)-7-[[4-[1-methyl-4-(trifluoromethyl)imidazol-2-yl]phenyl]methyl]-5-(2-trimethylsilylethoxymethyl)pyrrolo[3,2-d]pyrimidin-6-yl]methyl]-N-methyl-carbamate

To a solution of tert-butyl N-[[7-bromo-2-(2-cyclopropyl-3-pyridyl)-5-(2-trimethylsilylethoxymethyl)pyrrolo[3,2-d]pyrimidin-6-yl]methyl]-N-methyl-carbamate (150 mg, 255 μmol) in water (0.4 mL) and toluene (2 mL) were added potassium phosphate (108 mg, 510 μmol) and bis(triphenylphosphine)palladium(II) chloride (54 mg, 76 μmol) under nitrogen atmosphere at 25° C. The resulting mixture was stirred at 90° C. for 16 hours. The reaction was cooled down to room temperature, then quenched with water (100 mL), extracted with dichloromethane (3×50 mL). The combined organic layers were washed with brine (100 mL), dried over anhydrous sodium sulfate. After filtration, the filtrate was concentrated under reduced pressure. The residue was purified by silica gel column chromatography, eluted with 50%-60% ethyl acetate in petroleum ether to give tert-butyl N-[[2-(2-cyclopropyl-3-pyridyl)-7-[[4-[1-methyl-4-(trifluoromethyl)imidazol-2-yl]phenyl]methyl]-5-(2-trimethylsilylethoxymethyl)pyrrolo[3,2-d]pyrimidin-6-yl]methyl]-N-methyl-carbamate (50 mg, 66.85 μmol, 26% yield) as a yellow solid. MS: m/z=748.45 [M+H]⁺.

### 1.4 1-[2-(2-cyclopropyl-3-pyridyl)-7-[[4-[1-methyl-4-(trifluoromethyl)imidazol-2-yl]phenyl]methyl]-5H-pyrrolo[3,2-d]pyrimidin-6-yl]-N-methyl-methanamine

To a stirred solution of tert-butyl N-[[2-(2-cyclopropyl-3-pyridyl)-7-[[4-[1-methyl-4-(trifluoromethyl)imidazol-2-yl]phenyl]methyl]-5-(2-trimethylsilylethoxymethyl)pyrrolo[3,2-d]pyrimidin-6-yl]methyl]-N-methyl-carbamate (30 mg, 40.1 μmol) in methylene chloride (1 mL) was added trifluoroacetic acid (1 mL) and then stirred for 2 hours at 25° C. The resulted mixture was concentrated under reduced pressure. Then tetrahydrofuran (1 mL) and ammonium hydroxide (1 mL, 28% aqueous solution) were added in the above mixture, the resulted mixture was stirred for 30 min at 25° C. The resulted mixture was concentrated under reduced pressure. The residue was purified by Prep-TLC, eluted with 10% methanol in dichloromethane to give 20 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 50% B in 16 min, 50% B to 50% B in 4 min, 50% B to 95% B in 5 min; Detector: UV 254& 210 nm: RT: 24 min. The collected fractions were combined, concentrated and then lyophilized to afford 1-[2-(2-cyclopropyl-3-pyridyl)-7-[[4-[1-methyl-4-(trifluoromethyl)imidazol-2-yl]phenyl]methyl]-5H-pyrrolo[3,2-d]pyrimidin-6-yl]-N-methyl-methanamine (15.4 mg, 29.76 μmol, 74% yield) as a yellow solid. MS: m/z=518.35 [M+H]⁺. ¹H NMR (400 MHz, Chloroform-d) δ 9.56 (s, 1H), 8.92 (s, 1H), 8.50 (dd, J=4.8, 1.6 Hz, 1H), 8.04 (dd, J=7.6, 1.6 Hz, 1H), 7.49 (d, J=8.0 Hz, 2H), 7.39 (d, J=8.0 Hz, 2H), 7.31 (s, 1H), 7.15 (dd, J=7.6, 4.8 Hz, 1H), 4.28 (s, 2H), 3.97 (s, 2H), 3.74 (s, 3H), 2.83-2.77 (m, 1H), 2.43 (s, 3H), 1.20-1.16 (m, 2H), 0.88-0.84 (m, 2H). ¹⁹F NMR (376 MHz, Chloroform-d) δ −62.61.

### Example 107. 1-[2-(4-cyclopropyl-6-methoxy-pyrimidin-5-yl])-7-[[4-[1-methyl-4-(trifluoromethyl)imidazol-2-yl]phenyl]methyl]-5H-pyrrolo[3,2-d]pyrimidin-6-yl]-N,N-dimethyl-methanamine Compound 176

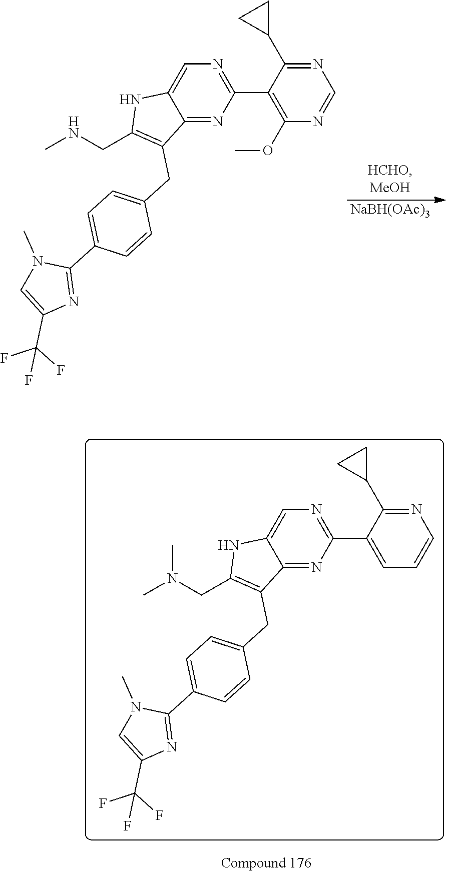 [SMILES: CNCc1[nH]c2cnc(-c3c(OC)ncnc3C3CC3)nc2c1Cc1ccc(-c2nc(C(F)(F)F)cn2C)cc1 ; CN(C)Cc1[nH]c2cnc(-c3cccnc3C3CC3)nc2c1Cc1ccc(-c2nc(C(F)(F)F)cn2C)cc1 ; "NaBH(OAc)3"]

### 1.1 1-[2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-6-yl]-N,N-dimethyl-methanamine

To a solution of 1-[2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-6-yl]-N-methyl-methanamine (30 mg, 55 μmol) in methanol (1 mL) was added formaldehyde aqueous solution (0.2 mL, 37%) then was stirred at 25° C. for 1 hour. The resulting solution was added sodium borohydride (10 mg, 273 μmol) at 25° C. The resulting solution was then stirred at 25° C. for 2 hours. The resulted solution was detected by LCMS, major desired product. The resulted solution was diluted with DCM (10 mL) and the solution was adjusted pH to 7˜8 using saturated sodium bicarbonate aqueous solution. The organic layer was separated, dried over anhydrous sodium sulfate. After filtration, the filtrate was concentrated under reduced pressure. The residue was purified by silica gel column chromatography, eluted with 0%-15% methanol in dichloromethane to give 20 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN: Flow rate: 60 mL/min; Gradient: 5% B to 5% B in 5 min, 5% B to 50% B in 13 min, 50% B to 50% B in 2 min, 50% B to 95% B in 5 min; Detector: UV 254& 210 nm; RT: 19 min. The collected fractions were combined, concentrated and then lyophilized to give [2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-6-yl]-N,N-dimethyl-methanamine (12.3 mg, 21.86 μmol, 40% yield) as an off-white solid. MS: m/z=563.40 [M+H]⁺. ¹H NMR (400 MHz, Chloroform-d) δ 9.47 (s, 1H), 8.97 (s, 1H), 7.50 (d, J=8.4 Hz, 2H), 7.40 (d, J=8.0 Hz, 2H), 7.31 (s, 1H), 4.29 (s, 2H), 3.95 (s, 3H), 3.73 (s, 3H), 3.72 (s, 2H), 2.33 (s, 6H), 1.79-1.73 (m, 1H), 1.30-1.19 (m, 2H), 0.88-0.84 (m, 2H). ¹⁹F NMR (377 MHz, Chloroform-d) δ −62.73.

### Example 108. 2-(4-cyclopropyl-6-ethoxy-pyrimidin-5-yl)-7-[[4-[1-methyl-4-(trifluoromethyl)imidazol-2-yl]phenyl]methyl]-5H-pyrrolo[3,2-d]pyrimidine Compound 111

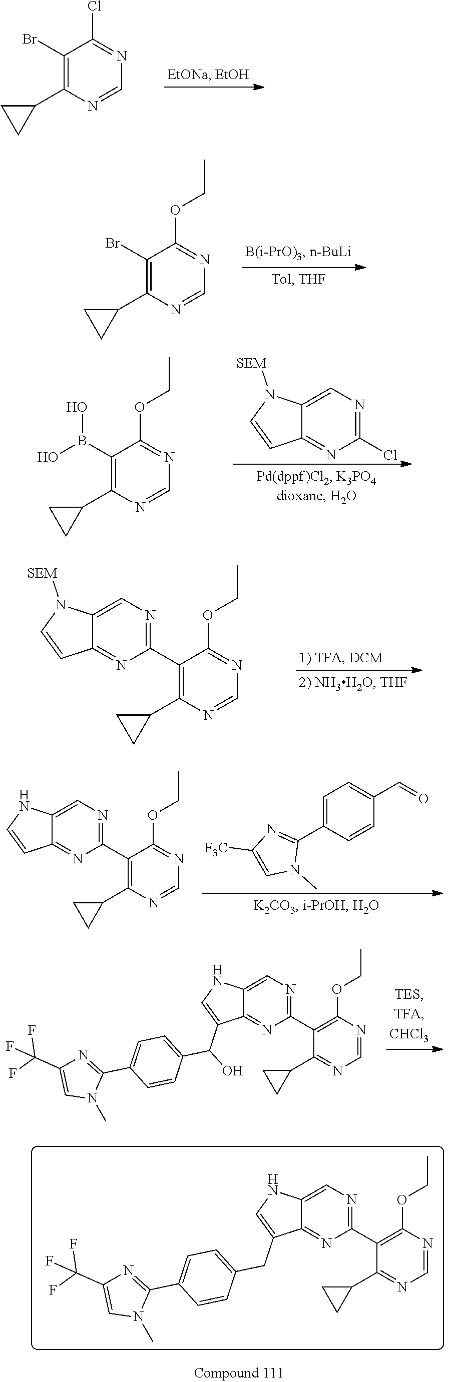

### 1.1 5-bromo-4-cyclopropyl-6-ethoxy-pyrimidine

To a solution of 5-bromo-4-chloro-6-cyclopropyl-pyrimidine (I g, 4.28 mmol) in ethanol (10 mL) was added sodium ethoxide (437 mg, 6.42 mmol) at 25° C., then stirred at 25° C. for 2 hours under nitrogen atmosphere. The reaction was detected by LCMS. The resulted reaction was quenched by saturated ammonium chloride aqueous solution (200 mL), extracted with ethyl acetate (3×200 mL). The combined organic layers were washed with brine (200 mL), dried over anhydrous sodium sulfate. After filtration, the filtrate was concentrated under reduced pressure. The residue was purified by silica gel column chromatography, eluted with 20%-25% ethyl acetate in petroleum ether. The collected fractions were combined and concentrated under reduced pressure to give 5-bromo-4-cyclopropyl-6-ethoxy-pyrimidine (800 mg, 3.29 mmol, 77% yield) as an off-white solid. MS: m/z=243.00, 245.00 [M+H]⁺. ¹H NMR (400 MHz, Chloroform-d) δ 8.42 (s, 1H), 4.52-4.47 (m, 2H), 2.57-2.51 (m, 1H), 1.46 (t, J=6.8 Hz, 3H), 1.22-1.17 (m, 2H), 1.13-1.07 (m, 2H).

### 1.2 (4-cyclopropyl-6-ethoxy-pyrimidin-5-yl)boronic Acid

To a stirred solution of 5-bromo-4-cyclopropyl-6-ethoxy-pyrimidine (50 mg, 206 μmol) in tetrahydrofuran (0.2 mL) and toluene (0.8 mL) was added triisopropyl borate (58.02 mg, 308.52 μmol) at room temperature under nitrogen atmosphere. The mixture was cooled down to −78° C. To the above mixture was slowly added n-butyllithium (148 μL, 370 μmol, 2.5 M in hexane) dropwise at −78° C. The resulting mixture was stirred for additional 15 min at −78° C. The resulted solution was quenched by 2 M aq. HCl (1 mL) then stirred for 1 hour at 25° C. under nitrogen, the solution was concentrated under reduced pressure. The mixture was purified by RP-Flash with the following conditions: Column: Spherical C18, 20-40 um, 330 g; Mobile Phase A: Water (10 mM aq. NH₄HCO₃), Mobile Phase B: MeCN; Flow rate: 100 mL/min; Gradient (B %): 0% B to 0% B in 10 min, 0% B to 12% B in 12 min, 12% B to 12% B in 15 min, 12% B to 95% B in 15 min; 95% B to 95% B in 5 min; Detector: UV 254 & 220 nm; RT: 30 min. The collected fractions were combined and concentrated under reduced pressure to afford (4-cyclopropyl-6-ethoxy-pyrimidin-5-yl)boronic acid as an off-white solid. MS: m/z=209.15 [M+H]⁺. ¹H NMR (400 MHz, Methanol-d₄) δ 8.50 (s, 1H), 4.44-4.33 (m, 2H), 2.08-1.87 (m, 1H), 1.41-1.36 (m, 3H), 1.09-0.99 (m, 4H).

### 1.3 2-[[2-(4-cyclopropyl-6-ethoxy-pyrimidin-5-yl)pyrrolo[3,2-d]pyrimidin-5-yl]methoxy]ethyl-trimethyl-silane

To a solution of (4-cyclopropyl-6-ethoxy-pyrimidin-5-yl)boronic acid (100 mg, 481 μmol) in 1,4-dioxane (2 mL) and water (0.4 mL) were added 1,1′-bis(diphenylphosphino)ferrocene-palladium(II)dichloride dichloromethane complex (39 mg, 48 μmol), potassium phosphate (102 mg, 481 μmol) and 2-[(2-chloropyrrolo[3,2-d]pyrimidin-5-yl)methoxy]ethyl-trimethyl-silane (136 mg, 481 μmol) at 25° C. The resulted mixture was stirred at 100° C. for 16 hours. The resulted mixture was cooled down to room temperature and the solid was filtered out. The filtrate was concentrated under reduced pressure. The residue was diluted with ethyl acetate (200 mL), washed with brine (3×50 mL). The organic layer was dried over anhydrous sodium sulfate. After filtration, the filtrate was concentrated under reduced pressure. The residue was purified by silica gel column chromatography, eluted with 0-20% ethyl acetate in petroleum ether to give 2-[[2-(4-cyclopropyl-6-ethoxy-pyrimidin-5-yl)pyrrolo[3,2-d]pyrimidin-5-yl]methoxy]ethyl-trimethyl-silane (100 mg, 242.97 μmol, 51% yield) as a yellow solid. MS: m/z=412.30 [M+H]⁺. ¹H NMR (400 MHz, Chloroform-d) δ 9.16 (s, 1H), 8.68 (d, J=4.0 Hz, 1H), 7.67 (s, 1H), 6.82 (s, 1H), 5.62 (s, 2H), 4.47-4.43 (m, 2H), 3.62-3.58 (m, 2H), 1.72-1.68 (m, 1H), 1.29-1.26 (s, 5H), 0.99-0.90 (m, 4H), 0.00 (s, 9H).

### 1.4 2-(4-cyclopropyl-6-ethoxy-pyrimidin-5-yl)-5H-pyrrolo[3,2-d]pyrimidine

To a stirred solution of 2-[[2-(4-cyclopropyl-6-ethoxy-pyrimidin-5-yl)pyrrolo[3,2-d]pyrimidin-5-yl]methoxy]ethyl-trimethyl-silane (50 mg, 121 μmol) in chloroform (1 mL) was trifluoroacetic acid (1 mL) and then stirred for 2 hours at 25° C. The resulted mixture was concentrated under reduced pressure, then tetrahydrofuran (1 mL) and ammonium hydroxide (1 mL) were added in the above mixture. The resulted mixture was stirred for 30 min at 25° C. The resulted mixture was concentrated under reduced pressure. The residue was purified by silica gel column chromatography, eluted with 50%-60% ethyl acetate in petroleum ether to afford 2-(4-cyclopropyl-6-ethoxy-pyrimidin-5-yl)-5H-pyrrolo[3,2-d]pyrimidine (25 mg, 88.87 μmol, 73% yield) as an off-white solid. MS: m/z=282.10 [M+H]⁺. ¹H NMR (400 MHz, Chloroform-d) S 9.39 (s, 1H), 9.12 (s, 1H), 8.65 (s, 1H), 7.73 (d, J=3.2 Hz, 1H), 6.86 (d, J=3.2 Hz, 1H), 4.44-4.39 (m, 2H), 1.73-1.66 (m, 1H), 1.28-1.25 (m, 3H), 1.24-1.22 (m, 2H), 0.94-0.85 (m, 2H).

### 1.5 [2-(4-cyclopropyl-6-ethoxy-pyrimidin-5-yl)-5H-pyrrolo[3,2-d]pyrimidin-7-yl]-[4-[1-methyl-4-(trifluoromethyl)imidazol-2-yl]phenyl]methanol

To a solution of 2-(4-cyclopropyl-6-ethoxy-pyrimidin-5-yl)-5H-pyrrolo[3,2-d]pyrimidine (50 mg, 177.74 μmol) in isopropyl alcohol (1 mL) and water (1 mL) was added potassium carbonate (49 mg, 355 μmol) and 4-[1-methyl-4-(trifluoromethyl)imidazol-2-yl]benzaldehyde (45 mg, 178 μmol) then stirred for 24 hours at 40° C. under nitrogen atmosphere. The reaction solution was detected by TLC and LCMS. The resulted mixture was diluted water (100 mL), extracted with ethyl acetate (3×60 mL). The combined organic layers were washed with brine (60 mL), dried over anhydrous sodium sulfate. After filtration, the filtrate was concentrated under reduced pressure. The residue was purified by Prep-TLC (CH₂Cl₂: MeOH=10:1) to give 50 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 47% B in 10 min, 47% B to 47% B in 2 min, 47% B to 95% B in 5 min; Detector: UV 254 & 210 nm; RT: 17 min. The collected fractions were combined, concentrated and then lyophilized to give [2-(4-cyclopropyl-6-ethoxy-pyrimidin-5-yl)-5H-pyrrolo[3,2-d]pyrimidin-7-yl]-[4-[1-methyl-4-(trifluoromethyl)imidazol-2-yl]phenyl]methanol (30 mg, 56.02 μmol, 32% yield) as an off-white solid. MS: m/z=536.35 [M+H]⁺. ¹H NMR (400 MHz, Methanol-d₄) δ 8.99 (s, 1H), 8.61 (s, 1H), 7.80 (s, 1H), 7.74-7.62 (m, 5H), 6.43 (s, 1H), 4.56-4.39 (m, 2H), 3.78 (s, 3H), 1.64-1.58 (m, 1H), 1.22 (t, J=6.8 Hz, 3H), 1.17-1.13 (m, 2H), 0.89-0.86 (m, 2H). ¹⁹F NMR (376 MHz, Methanol-d₄) δ −63.94.

### 1.6 2-(4-cyclopropyl-6-ethoxy-pyrimidin-5-yl)-7-[[4-[1-methyl-4-(trifluoromethyl)imidazol-2-yl]phenyl]methyl]-5H-pyrrolo[3,2-d]pyrimidine

To a solution of [2-(4-cyclopropyl-6-ethoxy-pyrimidin-5-yl)-5H-pyrrolo[3,2-d]pyrimidin-7-yl]-[4-[1-methyl-4-(trifluoromethyl)imidazol-2-yl]phenyl]methanol (30 mg, 56 μmol) in trichloromethane (1 mL) was added trifluoroacetic acid (0.5 mL) and triethyl silicane (1 mL) at 25° C., then was stirred at 25° C. for 16 hours. The resulted solution was detected by LCMS, major desired product. The resulted solution was concentrated under reduced pressure. The resulted residue was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 51% B in 12 min. 51% B to 51% B in 4 min. 51% B to 95% B in 5 min; Detector: UV 254 & 210 nm; RT: 19 min. The collected fractions were combined, concentrated and then lyophilized to give 2-(4-cyclopropyl-6-ethoxy-pyrimidin-5-yl)-7-[[4-[1-methyl-4-(trifluoromethyl)imidazol-2-yl]phenyl]methyl]-5H-pyrrolo[3,2-d]pyrimidine (4.7 mg, 9.05 μmol, 16% yield) as an off-white solid. MS: m/z=520.30 [M+H]⁺. ¹H NMR (400 MHz, Chloroform-d) δ 9.12 (s, 1H), 8.65 (s, 1H), 7.55 (d, J=8.0 Hz, 2H), 7.44 (d, J=8.0 Hz, 2H), 7.37-7.32 (m, 2H), 4.46-4.40 (m, 2H), 4.30 (s, 2H), 3.77 (s, 3H), 1.77-1.72 (m, 1H), 1.30-1.20 (m, 5H), 0.87-0.84 (m, 2H). ¹⁹F NMR (377 MHz, Chloroform-d) δ −62.59.

### Example 109. 5-(4-cyclopropyl-6-ethoxy-pyrimidin-5-yl)-3-[[4-[1-methyl-4-(trifluoromethyl)imidazol-2-yl]phenyl]methyl]-1H-pyrazolo[4,3-d]pyrimidine Compound 126

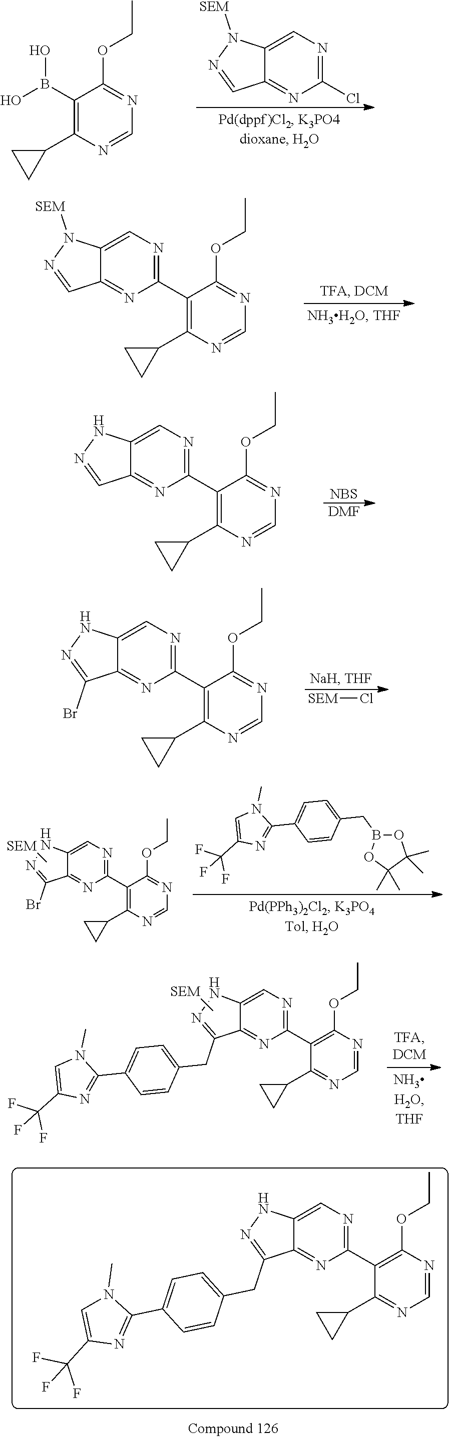

### 1.1 2-[[5-(4-cyclopropyl-6-ethoxy-pyrimidin-5-yl)pyrazolo[4,3-d]pyrimidin-1-yl]methoxy]ethyl-trimethyl-silane

To a solution of (4-cyclopropyl-6-ethoxy-pyrimidin-5-yl)boronic acid (350 mg, 1.68 mmol) and 2-[(5-chloropyrazolo[4,3-d]pyrimidin-1-yl)methoxy]ethyl-trimethyl-silane (479 mg, 1.68 mmol) in 1,4-dioxane (5 mL) and water (1 mL) were added 1,1′-bis(diphenylphosphino)ferrocene-palladium(II)dichloride dichloromethane complex (137 mg, 168 μmol) potassium phosphate (714 mg, 3.37 mmol) at 25° C. The resulted mixture was stirred at 100° C. for 16 hours. The resulted mixture was cooled down to room temperature and the solid was filtered out. The filtrate was concentrated under reduced pressure, then diluted with ethyl acetate (200 mL), washed with brine (3×50 mL). The organic layer was dried over anhydrous sodium sulfate. After filtration, the filtrate was concentrated under reduced pressure. The residue was purified by silica gel column chromatography, eluted with 0-20% ethyl acetate in petroleum ether to give 2-[[5-(4-cyclopropyl-6-ethoxy-pyrimidin-5-yl)pyrazolo[4,3-d]pyrimidin-1-yl]methoxy]ethyl-trimethyl-silane (180 mg, 436.30 μmol, 26% yield) as a yellow oil. MS: m/z =413.25. ¹H NMR (400 MHz, Chloroform-d) δ 9.42 (s, 1H), 8.68 (s, 1H), 8.34 (s, 1H), 5.89 (s, 2H), 4.46-4.41 (m, 2H), 3.71-3.63 (m, 2H), 1.68-1.62 (m, 1H), 1.27 (t, J=4.0 Hz, 3H), 1.26-1.24 (m, 2H), 0.99-0.89 (m, 4H), 0.01 (s, 9H).

### 1.2 5-(4-cyclopropyl-6-ethoxy-pyrimidin-5-yl)-1H-pyrazolo[4,3-d]pyrimidine

To a solution of 2-[[5-(4-cyclopropyl-6-ethoxy-pyrimidin-5-yl)pyrazolo[4,3-d]pyrimidin-1-yl]methoxy]ethyl-trimethyl-silane (180 mg, 436 μmol) in dichloromethane (2 mL) was added trifluoroacetic acid (2 mL) at 25° C., and then stirred at this temperature for 2 hours. The reaction solution was concentrated under reduced pressure. The residue was dissolved in tetrahydrofuran (2 mL) and ammonium hydroxide (2 mL) was added at 0° C. The resulted mixture was stirred at 25° C. for 0.5 hour and then concentrated under reduced pressure. The residue was purified by Prep-TLC, eluted with 7% methanol in dichloromethane to give 5-(4-cyclopropyl-6-ethoxy-pyrimidin-5-yl)-1H-pyrazolo[4,3-d]pyrimidine (100 mg, 354.23 μmol, 81% yield) as a yellow solid. MS: m/z=283.10 [M+H]⁺. ¹H NMR (400 MHz, Chloroform-d) δ 9.38 (s, 1H), 8.71 (s, 1H), 8.46 (s, 1H), 4.47-4.42 (m, 2H), 1.72-1.65 (m, 1H), 1.31-1.26 (m, 2H), 1.25 (t, J=7.2 Hz, 3H), 0.97-0.93 (m, 2H).

### 1.3 3-bromo-5-(4-cyclopropyl-6-ethoxy-pyrimidin-5-yl)-1H-pyrazolo[4,3-d]pyrimidine

To a stirred mixture of 5-(4-cyclopropyl-6-ethoxy-pyrimidin-5-yl)-1H-pyrazolo[4,3-d]pyrimidine (100 mg, 354 μmol) in N,N-dimethylformamide (1 mL) was added N-bromosuccinimide (63 mg, 354 μmol) under nitrogen atmosphere at 25° C. The resulting mixture was stirred for 2 hours at 25° C. The reaction was quenched by the addition of saturated ammonium chloride aqueous solution (100 mL). The resulted mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 45%-50% ethyl acetate in petroleum ether to give 3-bromo-5-(4-cyclopropyl-6-ethoxy-pyrimidin-5-yl)-1H-pyrazolo[4,3-d]pyrimidine (110 mg, 304.54 μmol, 86% yield) as a yellow oil. MS: m/z=361.00, 363.00 [M+H]⁺. ¹H NMR (400 MHz, Methanol-d₄) δ 9.41 (s, 1H), 8.63 (s, 1H), 4.45-4.39 (m, 2H), 1.62-1.61 (m, 1H), 1.24-1.16 (m, 5H), 0.95-0.89 (m, 2H).

### 1.4 2-[[3-bromo-5-(4-cyclopropyl-6-ethoxy-pyrimidin-5-yl)pyrazolo[4,3-d]pyrimidin-1-yl]methoxy]ethyl-trimethyl-silane & 2-[[3-bromo-5-(4-cyclopropyl-6-ethoxy-pyrimidin-5-yl)pyrazolo[4,3-d]pyrimidin-2-yl]methoxy]ethyl-trimethyl-silane

To a stirred mixture of 3-bromo-5-(4-cyclopropyl-6-ethoxy-pyrimidin-5-yl)-1H-pyrazolo[4,3-d]pyrimidine (100 mg, 276.86 μmol) in tetrahydrofuran (2 mL) was added sodium hydride (13 mg, 332 μmol, 60% purity) under nitrogen atmosphere at 0° C. The resulting mixture was stirred for 1 hour at 0° C. To the above mixture was added (2-(chloromethoxy)ethyl)trimethylsilane (55 mg, 332 μmol) under nitrogen atmosphere at 0° C. The resulted mixture was stirred at 25° C. for 2 hours. The reaction was quenched by the addition of saturated ammonium chloride aqueous solution (100 mL). The resulting mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 32%-33% ethyl acetate in petroleum ether to give a mixture of 2-[[3-bromo-5-(4-cyclopropyl-6-ethoxy-pyrimidin-5-yl)pyrazolo[4,3-d]pyrimidin-1-yl]methoxy]ethyl-trimethyl-silane and 2-[[3-bromo-5-(4-cyclopropyl-6-ethoxy-pyrimidin-5-yl)pyrazolo[4,3-d]pyrimidin-2-yl]methoxy]ethyl-trimethyl-silane (100 mg, 203.48 μmol, 22% yield) as a yellow oil. MS: m/z=491.25, 493.25 [M+H]⁺. ¹H NMR (400 MHz, Chloroform-d) δ 9.59, 9.41 (2s, 1H), 8.70 (s, 1H), 5.95, 5.85 (2s, 2H), 4.48-4.42 (m, 2H), 3.81-3.70 (m, 2H), 1.79-1.59 (m, 1H), 1.32-1.27 (m, 5H), 0.99-0.91 (m, 4H), 0.01 (s, 9H).

### 1.5 2-[[5-(4-cyclopropyl-6-ethoxy-pyrimidin-5-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane & 2-[[5-(4-cyclopropyl-6-ethoxy-pyrimidin-5-yl)-3-[[4-[1-methyl-4-(trifluoromethyl)imidazol-2-yl]phenyl]methyl]pyrazolo[4,3-d]pyrimidin-2-yl]methoxy]ethyl-trimethyl-silane

To a stirred solution of the mixture of 2-[[3-bromo-5-(4-cyclopropyl-6-ethoxy-pyrimidin-5-yl)pyrazolo[4,3-d]pyrimidin-1-yl]methoxy]ethyl-trimethyl-silane and 2-[[3-bromo-5-(4-cyclopropyl-6-ethoxy-pyrimidin-5-yl)pyrazolo[4,3-d]pyrimidin-2-yl]methoxy]ethyl-trimethyl-silane (100 mg, 203 μmol) in toluene (5 mL) and water (1 mL) were added bis(triphenylphosphine)palladium(II) chloride (43 mg, 61 μmol) and potassium phosphate (86 mg, 407 μmol) under nitrogen atmosphere at 25° C. The resulting mixture was stirred at 90° C. for 16 hours. The reaction was cooled down to room temperature, then quenched with water (100 mL), extracted with dichloromethane (3×50 mL). The combined organic layers were washed with brine (100 mL), dried over anhydrous sodium sulfate. After filtration, the filtrate was concentrated under reduced pressure. The residue was purified by silica gel column chromatography, eluted with 50%-60% ethyl acetate in petroleum ether to give a mixture of 2-[[5-(4-cyclopropyl-6-ethoxy-pyrimidin-5-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane & 2-[[5-(4-cyclopropyl-6-ethoxy-pyrimidin-5-yl)-3-[[4-[1-methyl-4-(trifluoromethyl)imidazol-2-yl]phenyl]methyl]pyrazolo[4,3-d]pyrimidin-2-yl]methoxy]ethyl-trimethyl-silane (50 mg, 76.83 μmol, 38% yield) as a yellow solid. MS: mV =651.40 [M+H]⁺.

### 1.6 5-(4-cyclopropyl-6-ethoxy-pyrimidin-5-yl)-3-[[4-[1-methyl-4-(trifluoromethyl)imidazol-2-yl]phenyl]methyl]-1H-pyrazolo[4,3-d]pyrimidine

To a stirred solution of the mixture of 2-[[5-(4-cyclopropyl-6-ethoxy-pyrimidin-5-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane & 2-[[5-(4-cyclopropyl-6-ethoxy-pyrimidin-5-yl)-3-[[4-[l-methyl-4-(trifluoromethyl)imidazol-2-yl]phenyl]methyl]pyrazolo[4,3-d]pyrimidin-2-yl]methoxy]ethyl-trimethyl-silane (50 mg, 77 μmol) in dichloromethane (1 mL) was added trifluoroacetic acid (1 mL) and then stirred for 2 hours at 25° C. The resulted mixture was concentrated under reduced pressure. Ammonium hydroxide (1 mL) and tetrahydrofuran (1 mL) were added in the above residue, the resulted mixture was stirred for 30 min at 25° C. The resulted mixture was concentrated under reduced pressure. The residue was purified by Prep-TLC, eluted with 10% methanol in dichloromethane to give 40 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 50% B in 13 min, 50% B to 50% B in 2 min, 50% B to 95% B in 5 min; Detector: UV 254& 210 nm; RT: 19 min. The collected fractions were combined, concentrated and then lyophilized to give 5-(4-cyclopropyl-6-ethoxy-pyrimidin-5-yl)-3-[[4-[1-methyl-4-(trifluoromethyl)imidazol-2-yl]phenyl]methyl]-1H-pyrazolo[4,3-d]pyrimidine (30 mg, 57.64 μmol, 75% yield) as an off-white solid. MS: m/z=521.30 [M+H]+. ¹H NMR (400 MHz, Chloroform-d) δ 13.60 (s, 1H), 9.16 (s, 1H), 8.67 (s, 1H), 7.69 (d, J=8.4 Hz, 2H), 7.59 (d, J=8.4 Hz, 2H), 7.42 (s, 1H), 4.55 (s, 2H), 4.44-4.38 (m, 2H), 3.83 (s, 3H), 1.68-1.61 (m, 1H), 1.25-1.20 (m, 5H), 0.86-0.81 (m, 2H). ¹⁹F NMR (376 MHz, Chloroform-d) δ −62.16.

### Example 110. 2-(4-cyclopropyl-6-methoxy-pyrimidin-5-yl)-7-[[3-fluoro-4-[5-methyl-3-(trifluoromethyl)pyrazol-1-yl]phenyl]methyl]-5H-pyrrolo[3,2-d]pyrimidine Compound 88

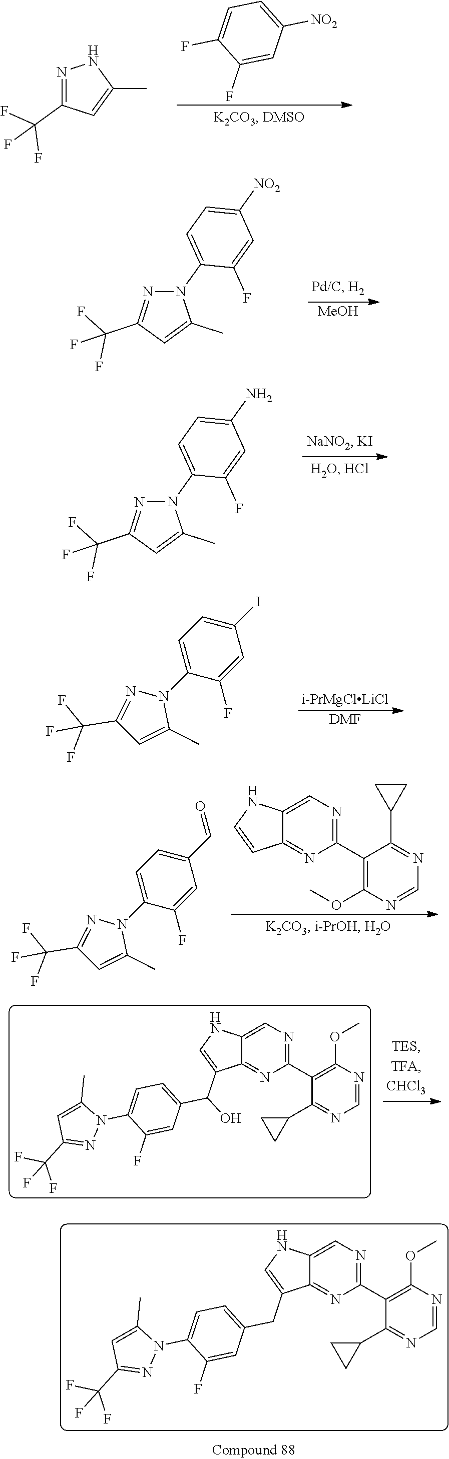

### 1.1 1-(2-fluoro-4-nitro-phenyl)-5-methyl-3-(trifluoromethyl)pyrazole

To a stirring solution of 5-methyl-3-(trifluoromethyl)-1H-pyrazole (7 g, 46.64 mmol) in dimethyl sulfoxide (70 mL) was added potassium carbonate (19.34 g, 139.9 mmol, 8.44 mL) under nitrogen atmosphere at 25° C. The resulting mixture was stirred under nitrogen atmosphere at 120° C. for 0.5 hour. To the above mixture was added 1,2-difluoro-4-nitro-benzene (11.13 g, 69.95 mmol, 7.74 mL) under nitrogen atmosphere at 120° C. The resulting mixture was stirred under nitrogen atmosphere at 120° C. for 4 hours. The reaction mixture was cooled down to room temperature. The reaction was monitored by LCMS. The reaction mixture was diluted with ethyl acetate (500 mL). The resulting mixture were washed with brine (3×500 mL), dried over anhydrous sodium sulfate. After filtration, the filtrate was concentrated under reduced pressure. The residue was purified by silica gel column chromatography, eluted with 3% methanol in dichloromethane to give 1-(2-fluoro-4-nitro-phenyl)-5-methyl-3-(trifluoromethyl)pyrazole (11 g, 38.04 mmol, 81% yield) as a yellow oil. MS: m/z=290.10 [M+H]⁺. ¹H NMR (400 MHz, Chloroform-d) δ 8.28-8.08 (m, 2H), 7.79-7.75 (m, 1H), 6.56 (s, 1H), 2.34 (s, 3H).

### 1.2 3-fluoro-4-[5-methyl-3-(trifluoromethyl)pyrazol-1-yl]aniline

To a stirred solution of 1-(2-fluoro-4-nitro-phenyl)-5-methyl-3-(trifluoromethyl)pyrazole (11 g, 38.04 mmol) in methanol (15 mL) was added palladium (2 g, 1.88 mmol, 10% purity) at room temperature under nitrogen atmosphere. The mixture was stirred under hydrogen atmosphere at room temperature for 2 hours, 90% desired product could be detected by LCMS. The resulted mixture were filtered, the filter cake was washed with methanol (3×50 mL). The filtrate was concentrated under reduced pressure to give 3-fluoro-4-[5-methyl-3-(trifluoromethyl)pyrazol-1-yl]aniline (8.7 g, 33.56 mmol, 88% yield) as a grey solid. The crude product was used in the next step directly without further purification. MS: m/z=260.10 [M+H]⁺. ¹H NMR (400 MHz, Chloroform-d) δ 7.22-7.16 (m, 1H), 6.60-6.46 (m, 2H), 6.44 (s, 1H), 2.23 (s, 3H). ¹⁹F NMR (376 MHz, Chloroform-d) δ −62.254, −121.355.

### 1.3 1-(2-fluoro-4-iodo-phenyl)-5-methyl-3-(trifluoromethyl)pyrazole

To a stirred solution of 3-fluoro-4-[5-methyl-3-(trifluoromethyl)pyrazol-1-yl]aniline (8.7 g, 33.56 mmol) in hydrochloric acid (50 mL) and water (50 mL) was added sodium nitrite (4.63 g, 67.13 mmol, 2.14 mL) dissolved with Water (50 mL) dropwise slowly at 0° C. for 1 hour. The mixture was stirred at 0° C. for 0.5 hour. To the above mixture was added potassium iodide (11.14 g, 67.13 mmol) dissolved with water (50 mL) dropwise at 0° C. The resulting mixture was stirred at 25° C. for additional 2 hours. The reaction was monitored by LCMS. The resulted mixture was extracted with ethyl acetate (3×200 mL). The combined organic layers were washed with brine (200 mL), dried over anhydrous sodium sulfate. After filtration, the filtrate was concentrated under reduced pressure. The residue was purified by silica gel column chromatography, eluted with 15% ethyl acetate in petroleum ether to give 1-(2-fluoro-4-iodo-phenyl)-5-methyl-3-(trifluoromethyl)pyrazole (12 g, 32.43 mmol, 96% yield) as a brown solid. MS: m/z=371.00 [M+H]⁺. ¹H NMR (400 MHz, Chloroform-d) δ 7.71-7.59 (m, 2H), 7.28-7.12 (m, 1H), 6.49 (s, 1H), 2.27 (s, 3H). ¹⁹F NMR (376 MHz, Chloroform-d) δ −62.578, −118.653.

### 1.4 3-fluoro-4-[5-methyl-3-(trifluoromethyl)pyrazol-1-yl]benzaldehyde

To a stirred solution of 1-(2-fluoro-4-iodo-phenyl)-5-methyl-3-(trifluoromethyl)pyrazole (4 g, 10.81 mmol) in tetrahydrofuran (50 mL) were added isopropylmagnesium chloride lithium chloride complex (24.94 mL, 32.43 mmol, 1.3 mol/L in THF) and N,N-dimethylformamide (7.90 g, 108.08 mmol, 8.37 mL) dropwise at room temperature under nitrogen atmosphere. The mixture was stirred under nitrogen atmosphere at room temperature atmosphere for 2 hours. The reaction was monitored by LCMS. The resulting mixture was concentrated under reduced pressure. The residue was purified by silica gel column chromatography, eluted with 27% ethyl acetate in petroleum ether to give 3-fluoro-4-[5-methyl-3-(trifluoromethyl)pyrazol-1-yl]benzaldehyde (2.4 g, 8.82 mmol, 81% yield) as a light yellow oil. MS: m/z=273.05 [M+H]⁺. ¹H NMR (400 MHz, Chloroform-d) δ 10.09 (s, 1H), 7.90-7.70 (m, 3H), 6.54 (s, 1H), 2.32 (s, 3H). ¹⁹F NMR (376 MHz, Chloroform-d) δ −62.573, −118.630.

### 1.5 [2-(4-cyclopropyl-6-methoxy-pyrimidin-5-yl)-5H-pyrrolo[3,2-d]pyrimidin-7-yl]-[3-fluoro-4-[5-methyl-3-(trifluoromethyl)pyrazol-1-yl]phenyl]methanol

To a stirring solution of 3-fluoro-4-[5-methyl-3-(trifluoromethyl)pyrazol-1-yl]benzaldehyde (600 mg, 2.20 mmol) in isopropyl alcohol (5.00 mL) was added 2-(4-cyclopropyl-6-methoxy-pyrimidin-5-yl)-5H-pyrrolo[3,2-d]pyrimidine (589 mg, 2.20 mmol) under nitrogen atmosphere at 0° C. The resulting mixture was stirred at 0° C. for 10 min. To the above mixture were added potassium carbonate (366 mg, 2.65 mmol,) and water (5.00 mL) at 0° C. The resulting mixture was stirred at 60° C. for 16 hours. The reaction mixture was cooled down to room temperature. The reaction was monitored by LCMS. The reaction mixture was diluted with water (100 mL), then extracted with ethyl acetate (3×100 mL). The combined organic layers were washed with brine (100 mL), dried over anhydrous sodium sulfate. After filtration, the filtrate was concentrated under reduced pressure. The residue was purified by silica gel column chromatography, eluted with 5%-6% methyl alcohol in dichloromethane to give 800 mg crude product. The crude product was purified by reverse phase chromatography with the following conditions: Column: C18 spherical Column, 20-35 um, 100A, 80 g; Mobile Phase A: Water (5 mM aq. NH₄HCO₃), Mobile Phase B: MeCN; Flow rate: 50 mL/min; Gradient: 5% B to 5% B in 5 min, 5% B to 60% B in 10 min, 60% B to 60% B in 4 min, 60% B to 95% B in 5 min; Detector: UV 254 & 210 nm; RT: 17 min. The product-containing fractions were collected, concentrated and then lyophilized overnight to give [2-(4-cyclopropyl-6-methoxy-pyrimidin-5-yl)-5H-pyrrolo[3,2-d]pyrimidin-7-yl]-[3-fluoro-4-[5-methyl-3-(trifluoromethyl)pyrazol-1-yl]phenyl]methanol (600 mg, 1.11 mmol, 50% yield) as an off-white solid. MS: m/z=540.30. [M+H]⁺. ¹H NMR (400 MHz, Chloroform-d) δ 9.58 (s, 1H), 9.06 (s, 1H), 8.67 (s, 1H), 7.54-7.46 (m, 3H), 6.51 (s, 1H), 6.41 (s, 1H), 3.93 (s, 3H), 2.27 (s, 3H), 1.78-1.66 (m, 1H), 1.26-1.22 (m, 2H), 0.93-0.87 (m, 2H). ¹⁹F NMR (376 MHz, Chloroform-d) δ −62.35, −120.59.

### 1.6 2-(4-cyclopropyl-6-methoxy-pyrimidin-5-yl)-7-[[3-fluoro-4-[5-methyl-3-(trifluoromethyl)pyrazol-1-yl]phenyl]methyl]-5H-pyrrolo[3,2-d]pyrimidine

To a stirred mixture of [2-(4-cyclopropyl-6-methoxy-pyrimidin-5-yl)-5H-pyrrolo[3,2-d]pyrimidin-7-yl]-[3-fluoro-4-[5-methyl-3-(trifluoromethyl)pyrazol-1-yl]phenyl]methanol (600 mg, 1.11 mmol) in chloroform (6 mL) were added trifluoroacetic acid (3 mL) and triethoxysilane (6 mL) under nitrogen atmosphere at 25° C. The resulting mixture was stirred at 25° C. for 12 hours. The reaction was monitored by LCMS. The resulted mixture was concentrated under reduced pressure. The residue was purified by reverse phase chromatography with the following conditions: C18 spherical Column, 20-35 um, 100A, 80 g; Mobile Phase A: Water (5 mM aq. NH₄HCO₃), Mobile Phase B: MeCN; Flow rate: 50 mL/min; Gradient: 5% B to 5% B in 5 min, 5% B to 60% B in 15 min, 60% B to 60% B in 3 min, 60% B to 95% B in 5 min; Detector: UV 254 & 210 nm; RT: 22 min. The product-containing fractions were collected, concentrated and then lyophilized overnight to give 2-(4-cyclopropyl-6-methoxy-pyrimidin-5-yl)-7-[[3-fluoro-4-[5-methyl-3-(trifluoromethyl)pyrazol-1-yl]phenyl]methyl]-5H-pyrrolo[3,2-d]pyrimidine (244.6 mg, 467.25 μmol, 42% yield) as an off-withe solid. MS: m/z=524.30 [M+H]⁺. ¹H NMR (400 MHz, Chloroform-d) δ 9.30 (s, 1H), 9.10 (s, 1H), 8.68 (s, 1H), 7.44-7.37 (m, 2H), 7.30 (s, 1H), 7.27 (s, 1H), 6.48 (s, 1H), 4.32 (s, 2H), 3.95 (s, 3H), 2.25 (s, 3H), 1.77-1.70 (m, 1H), 1.30-1.24 (m, 2H), 0.91-0.86 (m, 2H). ¹⁹F NMR (377 MHz, Chloroform-d) δ −62.37, −121.44.

### Example 111. 2-(4-chloro-2-cyclopropylpyridin-3-yl)-7-(4-(1-methyl-4-(trifluoromethyl)-1H-imidazol-2-yl)benzyl)-5H-pyrrolo[3,2-d]pyrimidine Compound 150

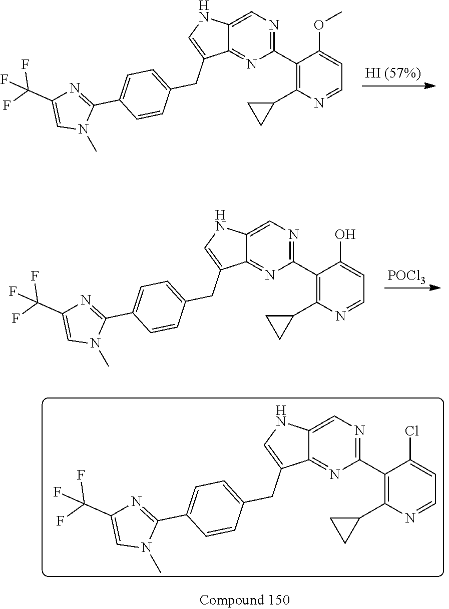

### 1.1 2-cyclopropyl-3-(7-(4-(1-methyl-4-(trifluoromethyl)-1H-imidazol-2-yl)benzyl)-5H-pyrrolo[3,2-d]pyrimidin-2-yl)pyridin-4-ol

2-(2-cyclopropyl-4-methoxypyridin-3-yl)-7-(4-(1-methyl-4-(trifluoromethyl)-1H-imidazol-2-yl)benzyl)-5H-pyrrolo[3,2-d]pyrimidine (70 mg, 139 μmol) was added Hydriodic acid (2 mL, 57%) at 25° C. under nitrogen atmosphere. The resulting mixture was stirred at 80° C. for 16 hours. The mixture was cooled down to room temperature. The residue was purified by Prep-TLC (dichloromethane:methanol=5:1) to afford 2-cyclopropyl-3-(7-(4-(1-methyl-4-(trifluoromethyl)-1H-imidazol-2-yl)benzyl)-5H-pyrrolo[3,2-d]pyrimidin-2-yl)pyridin-4-ol (25 mg, 50.97 μmol, 37% yield) as an off-white solid. MS: m/z=491.15 [M+H]⁺.

### 1.2 2-(4-chloro-2-cyclopropylpyridin-3-yl)-7-(4-(1-methyl-4-trifluoromethyl)-1H-imidazol-2-yl)benzyl)-5H-pyrrolo[3,2-d]pyrimidine

2-cyclopropyl-3-[7-[[4-[1-methyl-4-(trifluoromethyl)imidazol-2-yl]phenyl]methyl]-5H-pyrrolo[3,2-d]pyrimidin-2-yl]pyridin-4-ol (20 mg, 41 μmol) was added Phosphorus oxychloride (1.5 mL) at 25° C. under nitrogen atmosphere. The reaction mixture was stirred at 110° C. for 16 hours. The mixture was cooled down to room temperature. The solution was concentrated under reduced pressure. The residue was purified by Prep-TLC (dichloromethane:methanol=10:1) to give a crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 25 mL/min; Gradient: 5% B to 5% B in 5 min, 5% B to 55% B in 15 min, 55% B to 55% B in 1 min, 55% B to 95% B in 15 min; Detector: UV 254 & 210 nm; RT: 20 min. The collected fractions were combined, concentrated and then lyophilized overnight to afford 2-(4-chloro-2-cyclopropylpyridin-3-yl)-7-(4-(1-methyl-4-(trifluoromethyl)-1H-imidazol-2-yl)benzyl)-5H-pyrrolo[3,2-d]pyrimidine (4.5 mg, 8.84 μmol, 22% yield) as an off-white solid. MS: m/z=509.15 [M+H]⁺. ¹H NMR (400 MHz, Chloroform-d) δ 9.83 (s, 1H), 8.96 (s, 1H), 8.37 (d, J=5.4 Hz, 1H), 7.57-7.51 (m, 2H), 7.47-4.43 (m, 2H), 7.36 (s, 1H), 7.24-7.17 (m, 2H), 4.29 (s, 2H), 3.77 (s, 3H), 1.71-1.60 (m, 1H), 1.18-1.10 (m, 2H), 0.82-0.74 (m, 2H). ¹⁹F NMR (376 MHz, Chloroform-d) δ −62.507.

### Example 112. 2-(4-cyclopropyl-6-methoxy-pyrimidin-5-yl)-7-[[3,5-difluoro-4-[5-methyl-3-(trifluoromethyl)pyrazol-1-yl]phenyl]methyl]-5H-pyrrolo[3,2-d]pyrimidine Compound 162

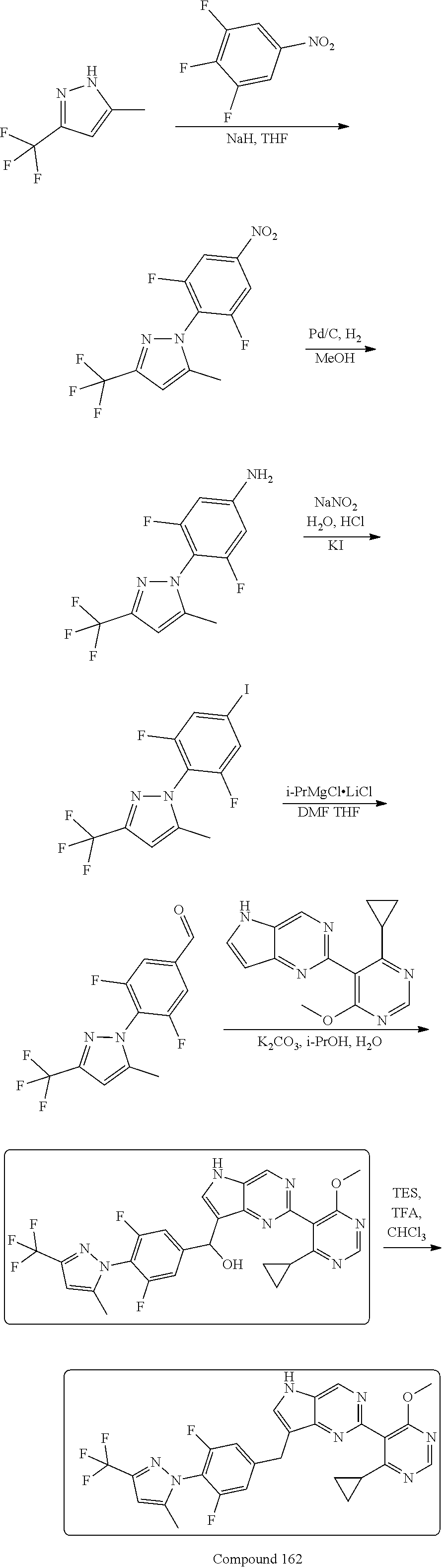

### 1.1 1-(2,6-difluoro-4-nitro-phenyl)-5-methyl-3-(trifluoromethyl)pyrazole

To a stirred solution of 5-methyl-3-(trifluoromethyl)-1H-pyrazole (5 g, 33.31 mmol) and 1,2,3-trifluoro-5-nitro-benzene (8.85 g, 49.97 mmol) in THF (50 mL) was added sodium hydride (2.66 g, 66.62 mmol, 60% in oil) at room temperature. The mixture was stirred at 70° C. for 2 hours. The reaction was quenched by the addition of saturated ammonium chloride aqueous solution (300 mL). The resulted mixture was extracted with ethyl acetate (3×100 mL). The combined organic layers were washed with brine (3×100 mL), dried over anhydrous sodium sulfate. After filtration, the filtrate was concentrated under reduced pressure. The residue was purified by silica gel column chromatography, eluted with ethyl acetate in petroleum ether (0%-15%) to afford 1-(2,6-difluoro-4-nitro-phenyl)-5-methyl-3-(trifluoromethyl)pyrazole (8.2 g, 26.69 mmol, 80% yield) as a light yellow oil. MS: no single in LCMS. ¹H NMR (400 MHz, Chloroform-d) δ 8.10-7.99 (m, 2H), 6.57 (s, 1H), 2.29 (s, 3H).

### 1.2 3,5-difluoro-4-[5-methyl-3-(trifluoromethyl)pyrazol-1-yl]aniline

To a stirred solution of 1-(2,6-difluoro-4-nitro-phenyl)-5-methyl-3-(trifluoromethyl)pyrazole (8 g, 26.04 mmol) in methanol (100 mL) was added palladium on carbon (1.6 g, 10% w/w) at room temperature. The mixture was stirred at room temperature for 2 hours under hydrogen atmosphere, 90% desired product could be detected by LCMS. The resulted mixture were filtered, the filter cake was washed with methanol (3×50 mL). The filtrate was concentrated under reduced pressure to afford 3,5-difluoro-4-[5-methyl-3-(trifluoromethyl)pyrazol-1-yl]aniline (7 g, 25.25 mmol, 96% yield) as a grey solid. The crude product was used in the next step directly without further purification. MS: m/z=278.05 [M+H]⁺. ¹H NMR (400 MHz, Chloroform-d) δ 6.46 (s, 1H), 6.33-6.27 (m, 2H), 4.18 (br., 2H), 2.24 (s, 3H).

### 1.3 1-(2,6-difluoro-4-iodo-phenyl)-5-methyl-3-(trifluoromethyl)pyrazole

To a stirred solution of 3,5-difluoro-4-[5-methyl-3-(trifluoromethyl)pyrazol-1-yl]aniline (9 g, 32.47 mmol) in concentrated hydrochloric acid (50 mL) and H₂O (50 mL) was added sodium nitrite (4.48 g, 64.94 mmol) dissolved with H₂O (50 mL) dropwise slowly at 0° C. for 0.5 hour. The mixture solution was stirred at 0° C. for 0.5 hour. To the above mixture was added potassium iodide (10.78 g, 64.94 mmol) dissolved with H₂O (50 mL) dropwise at 0° C. The resulting mixture was stirred at room temperature for additional 2 hours, 94% desired product could be detected by LCMS. The resulted mixture was extracted with ethyl acetate (3×200 mL). The combined organic layers were dried over anhydrous sodium sulfate. After filtration, the filtrate was concentrated under reduced pressure. The residue was purified by silica gel column chromatography, eluted with ethyl acetate in petroleum ether (0%-10%) to afford 1-(2,6-difluoro-4-iodo-phenyl)-5-methyl-3-(trifluoromethyl)pyrazole (11 g, 28.35 mmol, 87% yield) as a brown solid. MS: m/z=388.90 [M+H]⁺. ¹H NMR (400 MHz, Chloroform-d) δ 7.57-7.47 (m, 2H), 6.51 (s, 1H), 2.23 (s, 3H). ¹⁹F NMR (376 MHz, Chloroform-d) δ −62.639, −116.719.

### 1.4 3,5-difluoro-4-[5-methyl-3-(trifluoromethyl)pyrazol-1-yl]benzaldehyde

To a stirred solution of 1-(2,6-difluoro-4-iodo-phenyl)-5-methyl-3-(trifluoromethyl)pyrazole (2 g, 5.15 mmol) in THF (20 mL) were added isopropylmagnesium chloride lithium chloride complex (11.89 mL, 15.46 mmol, 1.3 mol/L in THF) and N,N-dimethylformamide (3.77 g, 51.54 mmol) dropwise at room temperature. The mixture solution was stirred at room temperature for 2 hours. The resulted mixture was concentrated under reduced pressure. The residue was purified by silica gel column chromatography, eluted with ethyl acetate in petroleum ether (0%-20%) to afford 3,5-difluoro-4-[5-methyl-3-(trifluoromethyl)pyrazol-1-yl]benzaldehyde (840 mg, 2.89 mmol, 56% yield) as a light yellow oil. MS: mm/z=291.00. ¹H NMR (400 MHz, Chloroform-d) δ 10.03 (s, 1H), 7.68-7.63 (m, 2H), 6.55 (s, 1H), 2.27 (s, 3H).

### 1.5 [2-(4-cyclopropyl-6-methoxy-pyrimidin-5-yl)-5H-pyrrolo[3,2-d]pyrimidin-7-yl]-[3,5-difluoro-4-[5-methyl-3-(trifluoromethyl)pyrazol-1-yl]phenyl]methanol

To a stirred solution of 3,5-difluoro-4-[5-methyl-3-(trifluoromethyl)pyrazol-1-yl]benzaldehyde (150 mg, 517 μmol) and 2-(4-cyclopropyl-6-methoxy-pyrimidin-5-yl)-5H-pyrrolo[3,2-d]pyrimidine (138 mg, 517 μmol) in i-PrOH (1 mL) and H₂O (1 mL) was added potassium carbonate (86 mg, 620 μmol) at room temperature. The mixture was stirred at 60° C. for 16 hours, 70% desired product could be detected by LCMS. The resulting mixture was concentrated under reduced pressure. The residue was purified by silica gel column chromatography, eluted with methanol in dichloromethane (0%-10%) to afford [2-(4-cyclopropyl-6-methoxy-pyrimidin-5-yl)-5H-pyrrolo[3,2-d]pyrimidin-7-yl]-[3,5-difluoro-4-[5-methyl-3-(trifluoromethyl)pyrazol-1-yl]phenyl]methanol (160 mg, 287.01 μmol, 55% yield) as a light yellow solid (160 mg).

The product (10 mg) was further purified by RP-Flash with the following conditions: Column: Spherical C18, 20-40 um, 20 g; Mobile Phase A: Water (5 mM aq. NH₄HCO₃), Mobile Phase B: ACN; Flow rate: 40 mL/min; Gradient (B %): 5% hold 5 min, 5%-41% within 20 min, 41% hold 5 min; 41%-95% within 10 min, 95% hold 5 min; Detector: UV 254 & 210 nm; RT: 27 min. The collected fractions were combined and concentrated under reduced pressure to afford [2-(4-cyclopropyl-6-methoxy-pyrimidin-5-yl)-5H-pyrrolo[3,2-d]pyrimidin-7-yl]-[3,5-difluoro-4-[5-methyl-3-(trifluoromethyl)pyrazol-1-yl]phenyl]methanol as an off-white solid (6.1 mg). MS: m/z=558.20. ¹H NMR (400 MHz, Methanol-d₄) δ 9.00 (s, 1H), 8.65 (s, 1H), 7.92 (s, 1H), 7.53-7.46 (m, 2H), 6.65 (s, 1H), 6.40 (s, 1H), 3.92 (s, 3H), 2.22 (d, J=8.0 Hz, 4H), 1.58-1.68 (m, 1H), 1.20-1.12 (m, 2H), 0.97-0.84 (m, 2H). ¹⁹F NMR (376 MHz, Methanol-d₄) δ −64.10, −121.12.

### 1.6 2-(4-cyclopropyl-6-methoxy-pyrimidin-5-yl)-7-[[3,5-difluoro-4-[5-methyl-3-(trifluoromethyl)pyrazol-1-yl]phenyl]methyl]-5H-pyrrolo[3,2-d]pyrimidine

To a stirred solution of [2-(4-cyclopropyl-6-methoxy-pyrimidin-5-yl)-5H-pyrrolo[3,2-d]pyrimidin-7-yl]-[3,5-difluoro-4-[5-methyl-3-(trifluoromethyl)pyrazol-1-yl]phenyl]methanol (120 mg, 215 μmol) in chloroform (2 mL) were added trifluoroacetic acid (1 mL) and triethylsilane (2 mL) at room temperature under air atmosphere. The mixture was stirred at room temperature for 2 hours, 70% desired product could be detected by LCMS. The resulting mixture was concentrated under reduced pressure. The residue was purified by silica gel column chromatography, eluted with methanol in dichloromethane (0%-10%) to afford product as a light yellow solid. The light yellow solid was purified by RP-Flash with the following conditions: Column: Spherical C18, 20-40 um, 40 g; Mobile Phase A: Water (5 mM NH₄HCO₃), Mobile Phase B: ACN; Flow rate: 40 mL/min; Gradient (B %): 5% hold 5 min, 5%-51% within 30 min, 51% hold 5 min; 51%-95% within 10 min, 95% hold 5 min; Detector: UV 254 & 210 nm; RT: 35 min. The collected fractions were combined and concentrated under reduced pressure and then lyophilized overnight to afford 2-(4-cyclopropyl-6-methoxy-pyrimidin-5-yl)-7-[[3,5-difluoro-4-[5-methyl-3-(trifluoromethyl)pyrazol-1-yl]phenyl]methyl]-5H-pyrrolo[3,2-d]pyrimidine (49 mg, 90.49 μmol, 42% yield) as an off-white solid. MS: m/z=542.10 [M+H]⁺. ¹H NMR (400 MHz, Methanol-d₄) δ 9.01 (s, 1H), 8.64 (s, 1H), 7.92 (s, 1H), 7.31-7.24 (m, 2H), 6.63 (s, 1H), 4.34 (s, 2H), 3.92 (s, 3H), 2.20 (s, 3H), 1.67-1.57 (m, 1H), 1.20-1.12 (m, 2H), 0.94-0.86 (m, 2H). ¹⁹F NMR (376 MHz, Methanol-d₄) δ −64.10, −121.79.

### Example 113. 2-(4-cyclopropyl-6-methoxy-pyrimidin-5-yl)-5-(2-methoxyethyl)-7-[[4-[1-methyl-4-(trifluoromethyl)imidazol-2-yl]phenyl]methyl]pyrrolo[3,2-d]pyrimidine Compound 200

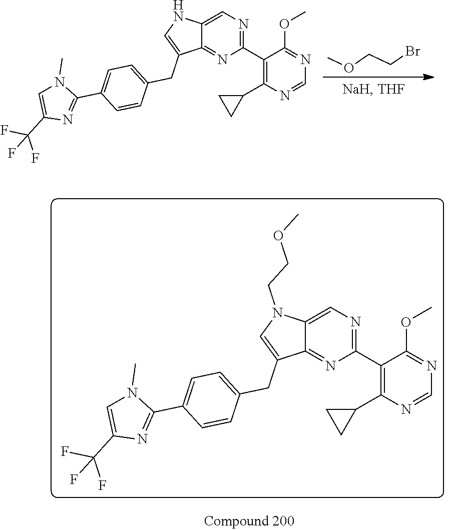

### 1.1 2-(4-cyclopropyl-6-methoxy-pyrimidin-5-yl)-5-(2-methoxyethyl)-7-[[4-[1-methyl-4-(trifluoromethyl)imidazol-2-yl]phenyl]methyl]pyrrolo[3,2-d]pyrimidine

To a solution of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (30 mg, 59.35 μmol) in tetrahydrofuran (3 mL) was added sodium hydride (36 mg, 891 μmol, 60% dispersion in oil) in portions at 0° C. under nitrogen atmosphere. The reaction mixture was stirred at 0° C. for 0.5 hour. Then 1-bromo-2-methoxy-ethane (74 mg, 534 μmol, 50 μL) was added to the reaction mixture at 25° C. under nitrogen atmosphere. The reaction mixture was stirred at 25° C. for 16 hours. The reaction was quenched with saturated ammonium chloride aqueous solution (20 mL), and then extracted with ethyl acetate (3×15 mL). The combined organic extracts were washed with brine (10 mL), dried over anhydrous sodium sulfate. After filtration, the filtrate was concentrated under reduced pressure. The residue was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 25 ml/min; Gradient: 5% B to 5% B in 5 min, 5% B to 55% B in 25 min, 55% B to 55% B in 2 min, 55% B to 95% B in 5 min; Detector: UV 254 & 210 nm; RT: 31 min. The collected fractions were combined, concentrated and then lyophilized overnight to give 2-(4-cyclopropyl-6-methoxy-pyrimidin-5-yl)-5-(2-methoxyethyl)-7-[[4-[1-methyl-4-(trifluoromethyl)imidazol-2-yl]phenyl]methyl]pyrrolo[3,2-d]pyrimidine (21.6 mg, 38.33 μmol, 64% yield) as an off-white solid. MS: m/z=564.25 [M+H]⁺. ¹H NMR (400 MHz, Chloroform-d) 9.03 (s, 1H), 8.68 (s, 1H), 7.58 (d, J=8.4 Hz, 2H), 7.48 (d, J=8.0 Hz, 2H), 7.33 (s, 1H), 7.21 (s, 1H), 4.34 (t, J=4.8 Hz, 2H), 4.29 (s, 2H), 3.96 (s, 3H), 3.78 (s, 3H), 3.74 (t, J=4.8 Hz, 2H), 3.37 (s, 3H), 1.76-1.71 (m, 1H), 1.28-1.23 (m, 2H), 0.90-0.85 (m, 2H). ¹⁹F NMR (376 MHz, Chloroform-d) δ −62.73.

### Example 114. 2-(4-cyclopropyl-6-methoxy-pyrimidin-5-yl)-7-[[4-[1-methyl-4-(trifluoromethyl)imidazol-2-yl]phenyl]methyl]-4-(trifluoromethyl)-5H-pyrrolo[3,2-d]pyrimidine Compound 121

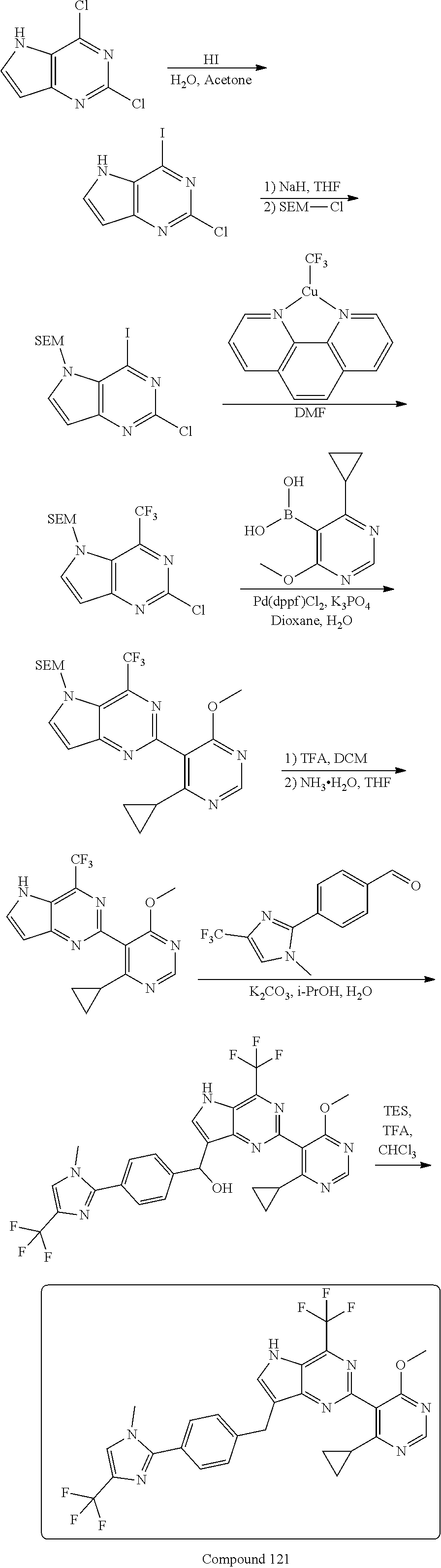

### 1.1 2-chloro-4-iodo-5H-pyrrolo[3,2-d]pyrimidine

To a mixture of 2,4-dichloro-5H-pyrrolo[3,2-d]pyrimidine (2 g, 10.64 mmol) in water (10 mL) and Acetone (10 mL) was added Hydriodic acid (57% in water) (30 mL) at 0° C. The resulting mixture was stirred at 35° C. for 20 hours. Major desired product was detected on LCMS. Then the mixture was quenched with saturated aqueous sodium bicarbonate (200 mL) at 0° C. The resulted mixture was extracted with ethyl acetate (3×200 mL). The combined organic layers was washed with brine (200 mL) and dried over anhydrous sodium sulfate. After filtration, the filtrate was concentrated under reduced pressure and the residue was purified by column chromatography on silica gel, eluted with 0-80% Ethyl acetate in Petroleum ether to afford 2-chloro-4-iodo-5H-pyrrolo[3,2-d]pyrimidine (2.8 g, 10.02 mmol, 94.19% yield) as a white solid. MS: m/z=279.95 [M+H]⁺. ¹H NMR (400 MHz, DMSO-d) δ 12.34 (s, 1H), 8.03 (t, J=3.2 Hz, 1H), 6.74-6.71 (m, 1H).

### 1.2 2-[(2-chloro-4-iodo-pyrrolo[3,2-d]pyrimidin-5-yl)methoxy]ethyl-trimethyl-silane

To a solution of 2-chloro-4-iodo-5H-pyrrolo[3,2-d]pyrimidine (1.59 g, 5.69 mmol) in tetrahydrofuran (20 mL) was added Sodium hydride (341 mg, 8.53 mmol, 60% dispersion in mineral oil) at 0° C. under nitrogen atmosphere. The mixture was stirred at 0° C. for 30 min. Then 2-(chloromethoxy)ethyl-trimethyl-silane (1.04 g, 6.26 mmol, 1.11 mL) was added at 0° C. under nitrogen atmosphere. The reaction mixture was stirred at rt, for 1.5 hours. Major desired product was detected on LCMS. The reaction was quenched with saturated ammonium chloride aqueous solution (50 mL), and then the mixture was extracted with ethyl acetate (3×50 mL). The combined organic phase was washed with brine (50 mL), dried over anhydrous sodium sulfate. After filtration, the filtrate was concentrated under reduced pressure and the residue was purified by silica gel column chromatography, eluted with 0-40% ethyl acetate in petroleum ether to give 2-[(2-chloro-4-iodo-pyrrolo[3,2-d]pyrimidin-5-yl)methoxy]ethyl-trimethyl-silane (1.73 g, 4.22 mmol, 74% yield) as a yellow solid. MS: m/z=410.00 [M+H]⁺. ¹H NMR (400 MHz, Chloroform-d): δ 7.63 (d, J=3.6 Hz, 1H), 6.61 (d, J=3.6 Hz, 1H), 5.79 (s, 2H), 3.54 (t, J=8.0 Hz, 2H), 0.92 (t, J=8.0 Hz, 2H), −0.03 (s, 9H).

### 1.3 2-[[2-chloro-4-(trifluoromethyl)pyrrolo[3,2-d]pyrimidin-5-yl]methoxy]ethyl-trimethyl-silane

A mixture of 2-[(2-chloro-4-iodo-pyrrolo[3,2-d]pyrimidin-5-yl)methoxy]ethyl-trimethyl-silane (810 mg, 1.98 mmol) and (1,10-phenanthroline)(trifluoromethyl)copper(I) (804 mg, 2.57 mmol) in N,N-dimethylformamide (13 mL) was stirred at room temperature for 2 hours under argon atmosphere. Major desired product was detected on LCMS. The reaction mixture was diluted with water (130 mL), extracted with ethyl acetate (5×100 mL). The combined organic phase was washed with brine (3×100 mL), dried over anhydrous sodium sulfate. After filtration, the filtrate was concentrated under reduced pressure and the residue was purified by reverse phase chromatography with the following conditions: Column: C18 gel column, 120 g, 20-35 μm; Mobile Phase A: 5 mM aq. NH₄HCO₃, Mobile Phase B: MeCN; Gradient: 0% B hold 10 min, up to 95% B within 35 min, 95% B hold 5 min; Flow rate: 80 mL/min; RT=37 min; Detector: UV 210 nm. The product-containing fractions were collected and evaporated to afford 2-[[2-chloro-4-(trifluoromethyl)pyrrolo[3,2-d]pyrimidin-5-yl]methoxy]ethyl-trimethyl-silane (600 mg, 1.71 mmol, 86% yield) as a yellow oil. MS: m/z=352.10 [M+H]⁺. ¹H NMR (400 MHz, Chloroform-d): δ 7.82 (d, J=3.6 Hz, 1H), 6.84 (d. J=3.6 Hz, 1H), 5.59 (s, 2H), 3.50 (t, J=8.0 Hz, 2H), 0.91 (t, J=8.0 Hz, 2H), −0.03 (s, 9H). ¹⁹F NMR (376 MHz, Chloroform-d) δ −63.655.

### 1.4 2-[[2-(4-cyclopropyl-6-methoxy-pyrimidin-5-yl)-4-(trifluoromethyl)pyrrolo[3,2-d]pyrimidin-5-yl]methoxy]ethyl-trimethyl-silane

To a solution of 2-[[2-chloro-4-(trifluoromethyl)pyrrolo[3,2-d]pyrimidin-5-yl]methoxy]ethyl-trimethyl-silane (565 mg, 1.61 mmol) and (4-cyclopropyl-6-methoxy-pyrimidin-5-yl)boronic acid (467.31 mg, 2.41 mmol) in 1,4-dioxane (6 mL) were added Potassium phosphate tribasic (682 mg, 3.21 mmol), [1,1′-Bis(diphenylphosphino)ferrocene]dichloropalladium(II) complex with dichloromethane (131 mg, 160.6 μmol) and water (1.2 mL) at room temperature under argon atmosphere. The resulting mixture was heated to 70° C., and stirred for 16 hours under argon atmosphere. Major desired product was detected on LCMS. Then the mixture was cooled to room temperature, diluted with water (100 mL), extracted with ethyl acetate (3×100 mL). The combined organic phase was washed with brine (50 mL), dried over anhydrous sodium sulfate. After filtration, the filtrate was concentrated under reduced pressure and the residue was purified by column chromatography on silica gel, eluted with 0-60% Ethyl acetate in Petroleum ether to afford 2-[[2-(4-cyclopropyl-6-methoxy-pyrimidin-5-yl)-4-(trifluoromethyl)pyrrolo[3,2-d]pyrimidin-5-yl]methoxy]ethyl-trimethyl-silane (400 mg, 859.21 μmol, 53%, yield) as a yellow solid. MS: m/z=466.10 [M+H]⁺. ¹H NMR (400 MHz, Chloroform-d) δ 8.66 (s, 1H), 7.83 (d, J=3.2 Hz, 1H), 6.96 (d, J=3.2 Hz, 1H), 5.65 (s, 2H), 3.91 (s, 3H), 3.58 (t, J=8.0 Hz, 2H), 1.68-1.59 (m, 1H), 1.27-1.22 (m, 2H), 0.95 (t, J=8.0 Hz, 2H), 0.92-0.86 (m, 2H), −0.01 (s, 9H). ¹⁹F NMR (377 MHz, Chloroform-d) δ −63.53.

### 1.5 2-(4-cyclopropyl-6-methoxy-pyrimidin-5-yl)-4-(trifluoromethyl)-5H-pyrrolo[3,2-d]pyrimidine

To a solution of 2-[[2-(4-cyclopropyl-6-methoxy-pyrimidin-5-yl)-4-(trifluoromethyl)pyrrolo[3,2-d]pyrimidin-5-yl]methoxy]ethyl-trimethyl-silane (400 mg, 859 μmol) in dichloromethane (6 mL) was added Trifluoroacetic acid (6 mL) at 0° C. The resulting mixture was stirred at room temperature for 2 hours. Then the mixture was concentrated under reduced pressure and the residue was re-dissolved in tetrahydrofuran (6 mL). To this solution was added ammonium hydroxide (6 mL) at room temperature. The resulting mixture was stirred at room temperature for 30 minutes. Desired product was detected on LCMS. Then the mixture was concentrated under reduced pressure and the residue was purified by column chromatography on silica gel, eluted with 0-60% Ethyl acetate in Petroleum ether to afford 2-(4-cyclopropyl-6-methoxy-pyrimidin-5-yl)-4-(trifluoromethyl)-5H-pyrrolo[3,2-d]pyrimidine (258 mg, 769.50 μmol, 89% yield) as a light-yellow solid. MS: m/z=336.15 [M+H]⁺. ¹H NMR (400 MHz, Chloroform-d) δ 9.30 (s, 1H), 8.66 (s, 1H), 7.82 (t, J=3.2 Hz, 1H), 6.96-6.93 (m, 1H), 3.90 (s, 3H), 168-1.59 (m, 1H), 1.31-1.20 (m, 2H), 0.96-0.83 (m, 2H). ¹⁹F NMR (377 MHz, Chloroform-d) δ −65.779.

### 1.6 [2-(4-cyclopropyl-6-methoxy-pyrimidin-5-yl)-4-(trifluoromethyl)-5H-pyrrolo[3,2-d]pyrimidin-7-yl]-[4-[1-methyl-4-(trifluoromethyl)imidazol-2-yl]phenyl]methanol

To a solution of 2-(4-cyclopropyl-6-methoxy-pyrimidin-5-yl)-4-(trifluoromethyl)-5H-pyrrolo[3,2-d]pyrimidine (210 mg, 626 μmol) and 4-[1-methyl-4-(trifluoromethyl)imidazol-2-yl]benzaldehyde (287 mg, 1.13 mmol) in Isopropyl alcohol (1.5 mL) were added Potassium Carbonate (173 mg, 1.25 mmol, 76 μL) and Water (1.5 mL) at room temperature under nitrogen atmosphere. The resulting mixture was stirred at 40° C. for 24 hours. Then the mixture was cooled to room temperature, diluted with water (10 mL), extracted with ethyl acetate (3×15 mL). The combined organic phase was washed with brine (20 mL), dried over anhydrous sodium sulfate. After filtration, the filtrate was concentrated under reduced pressure and the residue was purified by column chromatography on silica gel, eluted with 0-85% Ethyl acetate in Petroleum ether to afford [2-(4-cyclopropyl-6-methoxy-pyrimidin-5-yl)-4-(trifluoromethyl)-5H-pyrrolo[3,2-d]pyrimidin-7-yl]-[4-[1-methyl-4-(trifluoromethyl)imidazol-2-yl]phenyl]methanol (210 mg, 356.24 μmol, 57% yield) as a white solid, 40 mg of the product was further purified by reverse phase chromatography with the following conditions: Column: C18 gel column 40 g, 20-35 μm; Mobile Phase A: 5 mM aq. NH₄HCO₃, Mobile Phase B: MeCN: Gradient: 0% B hold 5 min, up to 60% B within 25 min, up to 95% B within 3 min, 95% B hold 10 min; Flow rate: 35 mL/min; RT=23 min; Detector: UV 254 & 210 nm. The product-containing fractions were collected and evaporated in vacuo and then lyophilized overnight to give 28.2 mg as a white solid. MS: m/z=590.30 [M+H]⁺. ¹H NMR (400 MHz, Chloroform-d) δ 9.36 (s, 1H), 8.73 (s, 1H), 7.67 (s, 4H), 7.38-7.33 (m, 2H), 6.43 (s, 1H), 3.96 (s, 3H), 3.80 (s, 3H), 1.79-1.68 (m, 1H), 1.36-1.27 (m, 2H), 0.98-0.90 (m, 2H). ¹⁹F NMR (377 MHz, Chloroform-d) δ −62.72, −65.94.

### 1.7 2-(4-cyclopropyl-6-methoxy-pyrimidin-5-yl)-7-[[4-[1-methyl-4-(trifluoromethyl)imidazol-2-yl]phenyl]methyl]-4-(trifluoromethyl)-5H-pyrrolo[3,2-d]pyrimidine

To a solution of [2-(4-cyclopropyl-6-methoxy-pyrimidin-5-yl)-4-(trifluoromethyl)-5H-pyrrolo[3,2-d]pyrimidin-7-yl]-[4-[1-methyl-4-(trifluoromethyl)imidazol-2-yl]phenyl]methanol (170 mg, 288 μmol) in dichloromethane (2 mL) were added Triethylsilane (2 mL) and Trifluoroacetic acid (1 mL) at 0° C. under nitrogen atmosphere. The reaction mixture was stirred at room temperature for 3 hours. Major desired product was detected on LCMS. Then the mixture was concentrated under reduced pressure. The residue was purified by reverse phase chromatography with the following conditions: Column: C18 spherical, 20-30 um, 100A, 40 g; Mobile Phase A: 5 mM aq. NH₄HCO₃, Mobile Phase B: MeCN; Flow rate: 40 mL/min; Gradient: 5% B hold 5 min, 5% B to 68% B in 20 min; 68% B hold 5 min; up to 95% B within 5 min; Detector: UV 254 & 210 nm; RT: 25 min. The product-containing fractions were collected and evaporated in vacuo and then lyophilized overnight to give 2-(4-cyclopropyl-6-methoxy-pyrimidin-5-yl)-7-[[4-[1-methyl-4-(trifluoromethyl)imidazol-2-yl]phenyl]methyl]-4-(trifluoromethyl)-5H-pyrrolo[3,2-d]pyrimidine (108 mg, 188.32 μmol, 65.30% yield) as an off-white solid. MS: m/z=574.30 [M+H]⁺. ¹H NMR (400 MHz, Chloroform-d) δ 9.32 (s, 1H), 8.71 (s, 1H), 7.57 (d, J=8.0 Hz, 2H), 7.48-7.42 (m, 3H), 7.34 (s, 1H), 4.31 (s, 2H), 3.95 (s, 3H), 3.78 (s, 3H), 1.74-1.66 (m, 1H), 1.33-1.24 (m, 2H), 0.94-0.85 (m, 2H). ¹⁹F NMR (377 MHz, Chloroform-d) δ −62.69, −65.81.

### Example 115. 5-(4-chloro-2-cyclopropylpyridin-3-yl)-3-(4-(1-methyl-4-(trifluoromethyl)-1H-imidazol-2-yl)benzyl)-1H-pyrazolo[4,3-d]pyrimidine Compound 156

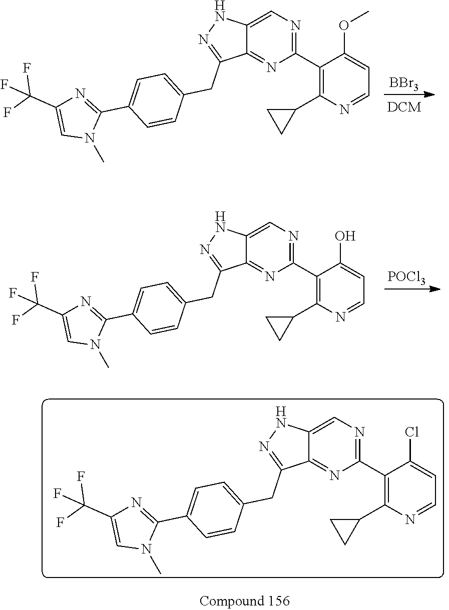

### 1.1 2-cyclopropyl-3-(3-(4-(1-methyl-4-(trifluoromethyl)-1H-imidazol-2-yl)benzyl)-1-pyrazolo[4,3-d]pyrimidin-5-yl)pyridin-4-ol

To a solution of 5-(2-cyclopropyl-4-methoxy-3-pyridyl)-3-[[4-[1-methyl-4-(trifluoromethyl)imidazol-2-yl]phenyl]methyl]-1H-pyrazolo[4,3-d]pyrimidine (17 mg, 34 μmol) in dichloromethane (0.5 mL) was added Boron tribromide (25 mg, 101 μmol). The resulting mixture was stirred at 40° C. for 3 hours. Product could be found by LCMS. The mixture was concentrated under reduced pressure. The residue was purified by Prep-TLC (Methanol: dichloromethane=1:7) to afford 2-cyclopropyl-3-(3-(4-(1-methyl-4-(trifluoromethyl)-1H-imidazol-2-yl)benzyl)-1H-pyrazolo[4,3-d]pyrimidin-5-yl)pyridin-4-ol (10 mg, 20.35 μmol, 61% yield) as off-white solid. MS: m/z=492.40 [M+H]⁺.

### 1.2 5-(4-chloro-2-cyclopropylpyridin-3-yl)-3-(4-(1-methyl-4-(trifluoromethyl)-1H-imidazol-2-yl)benzyl)-1H-pyrazolo[4,3-d]pyrimidine

The solution of 2-cyclopropyl-3-(3-(4-(1-methyl-4-(trifluoromethyl)-1H-imidazol-2-yl)benzyl)-1H-pyrazolo[4,3-d]pyrimidin-5-yl)pyridin-4-ol (10 mg, 20 μmol) in phosphorus oxychloride (1 mL) was stirred at 120° C. for 40 min at 25° C. under nitrogen atmosphere. The mixture was cooled down to room temperature. The mixture was concentrated under reduced pressure. The residue was purified by Prep-HPLC with the following conditions: Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 38% B to 48% B in 8 min, 48% B; Wave Length: 254 nm; RT1(min): 7. The collected fractions were combined, concentrated and then lyophilized overnight to afford 5-(4-chloro-2-cyclopropylpyridin-3-yl)-3-(4-(1-methyl-4-(trifluoromethyl)-1H-imidazol-2-yl)benzyl)-1H-pyrazolo[4,3-d]pyrimidine (4.4 mg, 8.63 μmol, 43% yield) as an off-white solid. MS: m/z=510.15 [M+H]⁺. ¹H NMR (400 MHz, Chloroform-d) δ 13.18 (s, 1H), 9.19 (s, 1H), 8.40 (d, J=5.2 Hz, 1H), 7.68 (d, J=8.0 Hz, 2H), 7.62 (d, J=8.0 Hz, 2H), 7.40 (s, 1H), 7.22 (d, J=5.2 Hz, 1H), 4.56 (s, 2H), 3.82 (s, 3H), 1.76-1.55 (m, 1H), 1.18-1.10 (m, 2H), 0.80-0.72 (m, 2H). ¹⁹F NMR (377 MHz, Chloroform-d) δ −62.20.

### Example 116. 2-[4-cyclopropyl-6-(fluoromethoxy)pyrimidin-5-yl]-7-[[4-[1-methyl-4-(trifluoromethyl)imidazol-2-yl]phenyl]methyl]-5H-pyrrolo[3,2-d]pyrimidine Compound 173

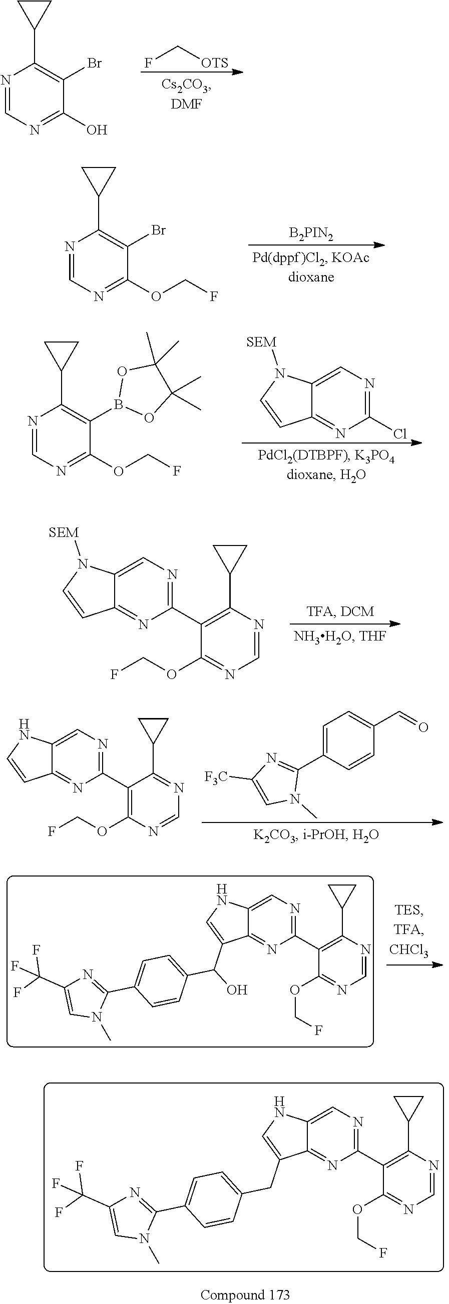

### 1.1 5-bromo-4-cyclopropyl-6-(fluoromethoxy)pyrimidine & 5-bromo-6-cyclopropyl-3-(fluoromethyl)pyrimidin-4-one

To a stirring solution of 5-bromo-6-cyclopropyl-pyrimidin-4-ol (10 g, 46.50 mmol) in N,N-dimethylformamide (100 mL) were added fluoromethyl 4-methylbenzenesulfonate (18.99 g, 93.00 mmol) and cesium carbonate (30.30 g, 93.00 mmol) under nitrogen atmosphere at 25° C. The resulting mixture was stirred at 60° C. for 1 hour. The reaction mixture was cooled to room temperature. The reaction was monitored by LCMS. The resulted mixture was diluted with ethyl acetate (1000 mL), washed with brine (2×200 mL), then dried over anhydrous sodium sulfate. After filtration, the filtrate was concentrated under reduced pressure. The residue was purified by silica gel column chromatography, eluted with 18% ethyl acetate in petroleum ether to give two isomers. The first eluting isomer was concentrated under reduced pressure to give 5-bromo-4-cyclopropyl-6-(fluoromethoxy)pyrimidine (3 g, 12.14 mmol, 26% yield) as an off-white solid. MS: m/z=247.05, 249.05 [M+H]⁺. ¹H NMR (400 MHz, Chloroform-d) δ 8.51 (s, 1H), 6.13 (d, J=51.2 Hz, 2H), 2.62-2.55 (m, 1H), 1.29-1.20 (m, 2H), 1.22-1.12 (m, 2H).

The second eluting isomer was concentrated under reduced pressure to give 5-bromo-6-cyclopropyl-3-(fluoromethyl)pyrimidin-4-one (3.2 g, 12.95 mmol, 27% yield) as an off-white solid. MS: m/z=247.00, 249.00 [M+H]⁺. ¹H NMR (400 MHz, Chloroform-d) δ 7.99 (s, 1H), 5.95 (d, J=50.0 Hz, 1H), 2.52-2.41 (m, 1H), 1.25-1.06 (m, 4H).

### 1.2 4-cyclopropyl-6-(fluoromethoxy)-5-(4,4,5,5-tetramethyl-1,3,2-dioxaborolan-2-yl)pyrimidine

To a stirring solution of 5-bromo-4-cyclopropyl-6-(fluoromethoxy)pyrimidine (1.5 g, 6.07 mmol) in 1,4-dioxane (15 mL) were added potassium acetate (1.79 g, 18.21 mmol), 4,4,5,5-tetramethyl-2-(4,4,5,5-tetramethyl-1,3,2-dioxaborolan-2-yl)-1,3,2-dioxaborolane (3.08 g, 12.14 mmol) and [1,1′-Bis(diphenylphosphino)ferrocene]dichloropalladium(II) complex with dichloromethane (496 mg, 607 μmol) under nitrogen atmosphere at 25° C. The resulting mixture was stirred at 95° C. for 16 hours. The reaction mixture was cooled down to room temperature.

The reaction was monitored by LCMS. After filtration, the filtrate was concentrated under reduced pressure. The residue was purified by RP-Flash with following conditions: C18 spherical Column, 20-35 um, 100A, 80 g; Mobile Phase A: Water (5 mM aq. NH₄HCO₃), Mobile Phase B: MeCN; Flow rate: 50 mL/min; Gradient: 5% B to 5% B in 5 min, 5% B to 60% B in 10 min. 60% B to 60% B in 3 min, 60% B to 95% B in 10 min; Detector: UV 254 & 210 nm: RT: 17 min. The product-containing fractions were collected, concentrated and then lyophilized overnight to give 4-cyclopropyl-6-(fluoromethoxy)-5-(4,4,5,5-tetramethyl-1,3,2-dioxaborolan-2-yl)pyrimidine (1.3 g 4.42 mmol, 64% yield) as an off-withe solid. MS: m/z=295.10 [M+H]⁺.

### 1.3 2-[[2-[4-cyclopropyl-6-(fluoromethoxy)pyrimidin-5-yl]pyrrolo[3,2-d]pyrimidin-5-yl]methoxy]ethyl-trimethyl-silane

To a stirring solution of 4-cyclopropyl-6-(fluoromethoxy)-5-(4,4,5,5-tetramethyl-1,3,2-dioxaborolan-2-yl)pyrimidine (1.3 g, 4.42 mmol) in 1,4-dioxane (10 mL) and water (2 mL) were added 2-[(2-chloropyrrolo[3,2-d]pyrimidin-5-yl)methoxy]ethyl-trimethyl-silane (1.25 g, 4.42 mmol), [1,1′-Bis(di-tert-butylphosphino)ferrocene]dichloropalladium(II) (289.37 mg, 441.98 μmol) and potassium phosphate (1.88 g, 8.84 mmol) under nitrogen atmosphere at 25° C. The resulting mixture was stirred at 90° C. for 16 hours. The reaction mixture was cooled down to room temperature. The reaction was monitored by LCMS. After filtration, the filtrate was concentrated under reduced pressure. The residue was purified by silica gel column chromatography, eluted with 4% methanol in dichloromethane to give 2-[[2-[4-cyclopropyl-6-(fluoromethoxy)pyrimidin-5-yl]pyrrolo[3,2-d]pyrimidin-5-yl]methoxy]ethyl-trimethyl-silane (750 mg, 1.80 mmol, 40% yield) as a yellow solid. MS: m/z=416.20 [M+H]⁺. ¹H NMR (400 MHz, Chloroform-d) δ 9.16 (s, 1H), 8.73 (s, 1H), 7.68 (d, J=3.6 Hz, 1H), 6.83 (d, J=3.2 Hz, 1H), 6.03 (d, J=51.2 Hz, 2H), 5.62 (s, 2H), 3.63-3.47 (m, 2H), 1.81-1.75 (m, 1H), 1.33-1.22 (m, 2H), 1.02-0.88 (m, 4H), 0.01-0.00 (m, 9H).

### 1.4 [2-(4,6-dimethoxypyrimidin-5-yl)-5H-pyrrolo[3,2-d]pyrimidin-7-yl]-[4-[1-methyl-4-(trifluoromethyl)imidazol-2-yl]phenyl]methanol

To a solution of 2-[[2-[4-cyclopropyl-6-(fluoromethoxy)pyrimidin-5-yl]pyrrolo[3,2-d]pyrimidin-5-yl]methoxy]ethyl-trimethyl-silane (750 mg, 1.80 mmol) in dichloromethane (10 mL) was added trifluoroacetic acid (10 mL) at 25° C., and then stirred at 25° C. for 2 hours. The starting material was disappeared on TLC. Then the reaction mixture was concentrated under reduced pressure. The residue was dissolved in tetrahydrofuran (10 mL) and ammonium hydroxide (10 mL, 28% aqueous solution) was added to the above mixture at 0° C. The reaction mixture was stirred at room temperature for 0.5 hour. The reaction was monitored by LCMS. The resulted mixture was concentrated under reduced pressure. The residue was purified by silica gel column chromatography, eluted with 5%-6% methanol in dichloromethane to give 2-[4-cyclopropyl-6-(fluoromethoxy)pyrimidin-5-yl]-5H-pyrrolo[3,2-d]pyrimidine (420 mg, 1.47 mmol, 81% yield) as a yellow solid. MS: m/z=286.15 [M+H]⁺.

### 1.5 [2-[4-cyclopropyl-6-(fluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol

To a stirring solution of 2-[4-cyclopropyl-6-(fluoromethoxy)pyrimidin-5-yl]-5H-pyrrolo[3,2-d]pyrimidine (370 mg, 1.30 mmol) in isopropyl alcohol (4 mL) under nitrogen atmosphere was added 4-[1-methyl-4-(trifluoromethyl)imidazol-2-yl]benzaldehyde (330 mg, 1.30 mmol) at 0° C. The resulting mixture was stirred at 0° C. for 10 min. To the above mixture were added potassium carbonate (215 mg, 1.56 mmol) and water (4 mL) at 0° C. The resulting mixture was stirred at 60° C. for 16 hours. The reaction mixture was cooled to room temperature. The reaction was monitored by LCMS. After filtration, the filtrate was concentrated under reduced pressure. The residue was purified by silica gel column chromatography, eluted with 3%-4% methanol in dichloromethane to give 200 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 25 mL/min; Gradient: 5% B to 5% B in 5 min, 5% B to 50% B in 10 min, 50% B to 50% B in 2 min, 50% B to 95% B in 5 min; Detector: UV 254 & 210 nm: RT: 16 min. The product-containing fractions were collected, concentrated and then lyophilized overnight to give [2-[4-cyclopropyl-6-(fluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol (100 mg, 185.36 μmol, 14% yield) as an off-white solid. MS: m/z=540.30. [M+H]⁺. ¹H NMR (400 MHz, Chloroform-4) δ 10.16 (br., 1H), 8.95 (s, 1H), 8.73 (s, 1H), 7.67 (d, J=8.0 Hz, 2H), 7.62 (d, J=8.4 Hz, 2H), 7.39 (s, 1H), 7.10 (s, 1H), 6.39 (s, 1H), 6.03 (d, J=51.6 Hz, 2H), 4.36 (br., 1H), 3.80 (s, 3H), 1.85-1.80 (m, 1H), 1.29-1.21 (m, 2H), 0.96-0.91 (m, 2H). ¹⁹F NMR (376 MHz, Chloroform-d) δ −62.47, −156.42.

### 1.6 2-[4-cyclopropyl-6-(fluoromethoxy)pyrimidin-5-yl]-7-[[4-[1-methyl-4-(trifluoromethyl)imidazol-2-yl]phenyl]methyl]-5H-pyrrolo[3,2-d]pyrimidine

To a stirred mixture of [2-[4-cyclopropyl-6-(fluoromethoxy)pyrimidin-5-yl]-5H-pyrrolo[3,2-d]pyrimidin-7-yl]-[4-[1-methyl-4-(trifluoromethyl)imidazol-2-yl]phenyl]methanol (100 mg, 185 μmol) in chloroform (2 mL) under nitrogen atmosphere were added trifluoroacetic acid (1 mL) and triethoxysilane (2 mL) at 25° C. The reaction mixture was stirred at 25° C. for 2 hours. The reaction was monitored by LCMS. The resulted mixture was concentrated under reduced pressure. The residue was purified by RP-Flash with the following conditions: C18 spherical Column, 20-35 um, 100A, 40 g; Mobile Phase A: Water (5 mM aq. NH₄HCO₃), Mobile Phase B: MeCN: Flow rate: 25 mL/min; Gradient: 5% B to 5% B in 5 min, 5% B to 53% B in 13 min, 53% B to 53% B in 2 min, 53% B to 95% B in 5 min; Detector: UV 254 & 210 nm; RT: 19 min. The product-containing fractions were collected, concentrated and then lyophilized overnight to give 2-[4-cyclopropyl-6-(fluoromethoxy)pyrimidin-5-yl]-7-[[4-[1-methyl-4-(trifluoromethyl)imidazol-2-yl]phenyl]methyl]-5H-pyrrolo[3,2-d]pyrimidine (18.3 mg, 34.96 μmol, 18% yield) as an off-withe solid. MS: m/z=524.30 [M+H]⁺. ¹H NMR (400 MHz, Chloroform-d) δ 10.33 (s, 1H), 8.93 (s, 1H), 8.71 (s, 1H), 7.54 (d, J=8.0 Hz, 2H), 7.44 (d, J=8.0 Hz, 2H), 7.37 (s, 1H), 7.20 (s, 1H), 6.01 (d, J=51.2 Hz, 2H), 4.28 (s, 2H), 3.78 (s, 3H), 1.84-1.77 (m, 1H), 1.28-1.24 (m, 2H), 0.95-0.87 (m, 2H). ¹⁹F NMR (376 MHz, Chloroform-d) δ −62.44, −156.55.

### Example 117. 5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)-1H-pyrazolo[4,3-d]pyrimidine Compound 114

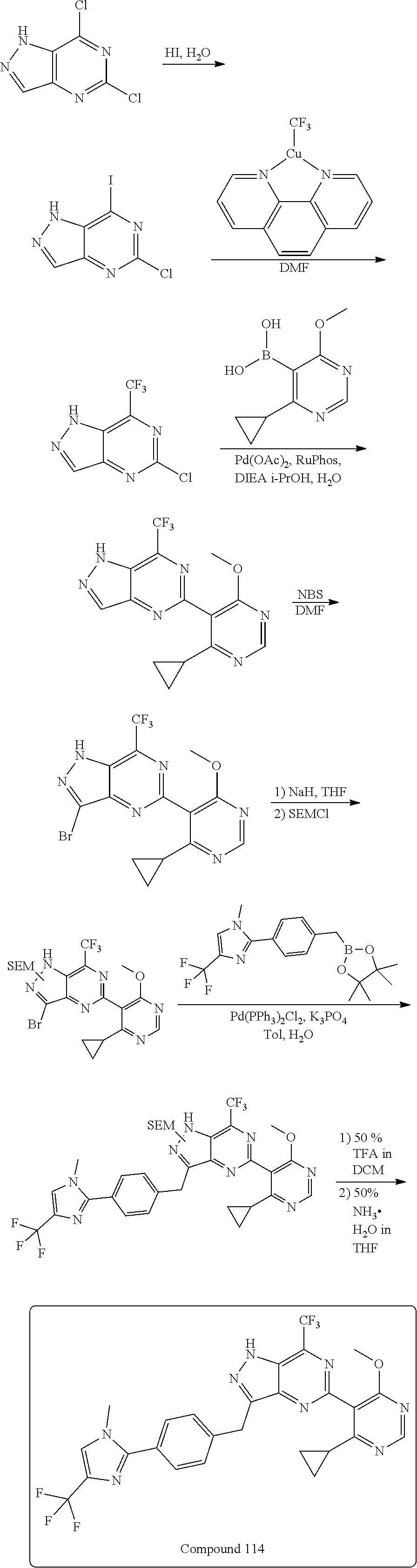

### 1.1 5-chloro-7-iodo-1H-pyrazolo[4,3-d]pyrimidine

5,7-dichloro-1H-pyrazolo[4,3-d]pyrimidine (2.4 g, 12.70 mmol) was added to Hydriodic acid (57% in water, 35 mL) at 0° C. The reaction mixture was stirred at 0° C. for 15 min. Then the mixture was poured into saturated sodium bicarbonate aqueous solution (100 mL), and extracted with ethyl acetate (3×100 mL). The combined organic phase was washed with brine (2×50 mL), dried over anhydrous sodium sulfate. After filtration, the filtrate was concentrated under reduced pressure and the residue was purified by column chromatography on silica gel, eluted with 0-50% Ethyl acetate in Petroleum ether to afford to afford 5-chloro-7-iodo-1H-pyrazolo[4,3-d]pyrimidine (2.7 g, 9.63 mmol, 76% yield) as a yellow solid. MS: m/z=280.90 [M+H]⁺. ¹H NMR (400 MHz, DMSO-d₆) δ 14.49 (s, 1H), 8.55 (s, 1H).

### 1.2 5-chloro-7-(trifluoromethyl)-1H-pyrazolo[4,3-d]pyrimidine

To a solution of 5-chloro-7-iodo-1H-pyrazolo[4,3-d]pyrimidine (800 mg, 2.85 mmol) in DMF (7 mL) was added (1,10-phenanthroline)(trifluoromethyl)copper (I) (1.34 g, 4.28 mmol) at room temperature under nitrogen atmosphere. The mixture was stirred at 35° C. for 24 h then diluted with water (25 mL), extracted with ethyl acetate (4×30 mL). The combined organic phase was washed with brine (4×25 mL), dried over anhydrous sodium sulfate. After filtration, the filtrate was concentrated under reduced pressure and the residue was purified by column chromatography on silica gel, eluted with 0-60% Ethyl acetate in Petroleum ether to afford 5-chloro-7-(trifluoromethyl)-1H-pyrazolo[4,3-d]pyrimidine (230 mg, 1.03 mmol, 36% yield) as a yellow solid. MS: m/z=221.05 [M−H]⁻. ¹H NMR (400 MHz, Chloroform-d) δ 8.46 (s, 1H). ¹⁹F NMR (377 MHz, Chloroform-d): δ −66.64.

### 1.3 5-(4-cyclopropyl-6-methoxy-pyrimidin-5-yl)-7-(trifluoromethyl)-1H-pyrazolo[4,3-d]pyrimidine

To a solution of 5-chloro-7-(trifluoromethyl)-1H-pyrazolo[4,3-d]pyrimidine (155 mg, 696.46 μmol) and (4-cyclopropyl-6-methoxy-pyrimidin-5-yl)boronic acid (203 mg, 1.04 mmol) in isopropyl alcohol (2 mL) and water (0.4 mL) were added Palladium (II) Acetate (16 mg, 70 μmol), 2-Dicyclohexylphosphino-2,6-diisopropoxybiphenyl (65 mg, 139 μmol) and N,N-diisopropylethylamine (180 mg, 1.39 mmol) at room temperature under argon atmosphere. The resulted mixture was stirred at 90° C. for 4 hours then cooled to room temperature, diluted with brine (10 mL), extracted with ethyl acetate (3×15 mL). The combined organic phase was washed with brine (2×10 mL), dried over anhydrous sodium sulfate. After filtration, the filtrate was concentrated under reduced pressure. The residue (combined from three batches, theoretical 100% yield assumed, 966 μmol) was purified by preparative TLC (developed by Ethyl acetate/Petroleum ether=1/3) to afford 5-(4-cyclopropyl-6-methoxy-pyrimidin-5-yl)-7-(trifluoromethyl)-1H-pyrazolo[4,3-d]pyrimidine (145 mg, 431.20 μmol, 45% yield) as a yellow solid. MS: m/z=337.05 [M+H]⁺. ¹H NMR (400 MHz, Chloroform-d): δ 11.25 (s, 1H), 8.69 (s, 1H), 8.56 (s, 1H), 3.92 (s, 3H), 1.67-1.57 (m, 1H), 1.27-1.10 (m, 2H), 0.96-0.88 (m, 2H). ¹⁹F NMR (377 MHz, Chloroform-d): δ −66.49.

### 1.4 3-bromo-5-(4-cyclopropyl-6-methoxy-pyrimidin-5-yl)-7-(trifluoromethyl)-1H-pyrazolo[4,3-d]pyrimidine

To a solution of 5-(4-cyclopropyl-6-methoxy-pyrimidin-5-yl)-7-(trifluoromethyl)-1H-pyrazolo[4,3-d]pyrimidine (130 mg, 386.5 μmol) in DMF (2 mL) was added N-Bromosuccinimide (103 mg, 580 μmol) at room temperature under nitrogen atmosphere. The reaction mixture was stirred at room temperature for 3 hours then diluted with water (10 mL), extracted with ethyl acetate (3×10 mL). The combined organic phase was washed with brine (2×5 mL), dried over anhydrous sodium sulfate. After filtration, the filtrate was concentrated under reduced pressure. The residue (combined from two batches, theoretical 100% yield assumed, 416 μmol) was purified by column chromatography on silica gel, eluted with 0-60% Ethyl acetate in Petroleum ether to afford 3-bromo-5-(4-cyclopropyl-6-methoxy-pyrimidin-5-yl)-7-(trifluoromethyl)-1H-pyrazolo[4,3-d]pyrimidine (160 mg, 385.39 μmol, 92% yield) as a yellow solid. MS: m/z=415.00, 416.95 [M+H]⁺. ¹H NMR (400 MHz, Chloroform-d) δ 8.72 (s, 1H), 3.93 (s, 3H), 1.65-1.58 (m, 1H), 1.36-1.29 (m, 2H), 0.99-0.93 (m, 2H). ¹⁹F NMR (376 MHz, Chloroform-d) δ −66.34.

### 1.5 2-[[3-bromo-5-(4-cyclopropyl-6-methoxy-pyrimidin-5-yl)-7-(trifluoromethyl)pyrazolo[4,3-d]pyrimidin-1-yl]methoxy]ethyl-trimethyl-silane & 2-[[3-bromo-5-(4-cyclopropyl-6-methoxy-pyrimidin-5-yl)-7-(trifluoromethyl)pyrazolo[4,3-d]pyrimidin-2-yl]methoxy]ethyl-trimethyl-silane

To a solution of 3-bromo-5-(4-cyclopropyl-6-methoxy-pyrimidin-5-yl)-7-(trifluoromethyl)-1H-pyrazolo[4,3-d]pyrimidine (147 mg, 354 μmol) in THF (2 mL) was added sodium hydride (16 mg, 389 μmol, 60% dispersion in mineral oil) at 0° C. under argon atmosphere. The mixture was stirred at 0° C. for 1 hour under argon atmosphere then 2-(trimethylsilyl)ethoxymethyl chloride (65 mg, 389 μmol) was added. The resulting mixture was stirred at room temperature for 2 hours. The reaction was quenched with saturated ammonium chloride aqueous solution (5 mL), extracted with ethyl acetate (3×5 mL). The combined organic phase was washed with brine (8 mL), dried over anhydrous sodium sulfate. After filtration, the filtrate was concentrated under reduced pressure. The residue (combined from two batches, theoretical 100% yield assumed, 378 μmol) was purified by column chromatography on silica gel, eluted with 0-55% Ethyl acetate in Petroleum ether to give a mixture of 2-[[3-bromo-5-(4-cyclopropyl-6-methoxy-pyrimidin-5-yl)-7-(trifluoromethyl)pyrazolo[4,3-d]pyrimidin-1-yl]methoxy]ethyl-trimethyl-silane and 2-[[3-bromo-5-(4-cyclopropyl-6-methoxy-pyrimidin-5-yl)-7-(trifluoromethyl)pyrazolo[4,3-d]pyrimidin-2-yl]methoxy]ethyl-trimethyl-silane (165 mg, 302.52 μmol, 80% yield) as a yellow oil. MS: m/z=545.05, 547.00 [M+H]⁺.

### 1.6 2-[[5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)pyrazolo[4,3-d]pyrimidin-1-yl]methoxy]ethyl-trimethyl-silane & 2-[[5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)pyrazolo[4,3-d]pyrimidin-2-yl]methoxy]ethyl-trimethyl-silane

To a stirred solution of 2-[[3-bromo-5-(4-cyclopropyl-6-methoxy-pyrimidin-5-yl)-7-(trifluoromethyl)pyrazolo[4,3-d]pyrimidin-1-yl]methoxy]ethyl-trimethyl-silane & 2-[[3-bromo-5-(4-cyclopropyl-6-methoxy-pyrimidin-5-yl)-7-(trifluoromethyl)pyrazolo[4,3-d]pyrimidin-2-yl]methoxy]ethyl-trimethyl-silane (160 mg, a mixture of two isomers) and 1-methyl-2-[4-[(4,4,5,5-tetramethyl-1,3,2-dioxaborolan-2-yl)methyl]phenyl]-4-(trifluoromethyl)imidazole (107 mg, 292 μmol) in toluene (2 mL) and water (0.4 mL) were added potassium phosphate (124 mg, 584 μmol) and bis(triphenylphosphine)palladium (II) chloride (61.5 mg, 88 μmol) at 25° C. under nitrogen atmosphere. The resulting mixture was stirred for 16 hours at 100° C. under nitrogen atmosphere then filtered. The filter cake was washed with ethyl acetate (3×3 mL). The filtrate was concentrated under reduced pressure. The residue was purified by Prep-TLC (50% ethyl acetate in petroleum ether) to afford 2-[[5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)pyrazolo[4,3-d]pyrimidin-1-yl]methoxy]ethyl-trimethyl-silane & 2-[[5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)pyrazolo[4,3-d]pyrimidin-2-yl]methoxy]ethyl-trimethyl-silane (70 mg, a mixture of two isomers) as a brown solid. MS: m/z=705.20 [M+H]⁺.

### 1.7 5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)-1H-pyrazolo[4,3-d]pyrimidine

To a stirred solution of 2-[[5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)pyrazolo[4,3-d]pyrimidin-1-yl]methoxy]ethyl-trimethyl-silane and 2-[[5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)pyrazolo[4,3-d]pyrimidin-2-yl]methoxy]ethyl-trimethyl-silane (70 mg, a mixture of two isomers) in DCM (2 mL) was added TFA (2.96 g, 25.96 mmol, 2 mL) at 25° C. under nitrogen atmosphere. The resulting mixture was stirred for 1 hour at 25° C. under nitrogen atmosphere then concentrated under reduced pressure. To the stirred solution of the residue in THF (2 mL) was added ammonium hydroxide (1.80 g, 51.36 mmol, 2 mL) at 25° C. under nitrogen atmosphere. The resulting mixture was stirred for 1 hour at 25° C. under nitrogen atmosphere then concentrated under reduced pressure. The residue was purified by silica gel column chromatography, eluted with 1%-20% methanol in dichloromethane to afford crude product. The obtained crude product was further purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 45% B in 20 min, 45% B hold in 3 min, up to 95% B in 5 min; Detector: UV 254 & 210 nm: RT: 27 min. The collected fractions were combined and lyophilized to give 5-(4-cyclopropyl-6-methoxy-pyrimidin-5-yl)-3-[[4-[1-methyl-4-(trifluoromethyl)imidazol-2-yl]phenyl]methyl]-7-(trifluoromethyl)-1H-pyrazolo[4,3-d]pyrimidine (33.9 mg, 59.01 μmol, 59% yield) as a white solid. MS: m/z=575.10 [M+H]⁺. ¹H NMR (400 MHz, Chloroform-d) δ 8.73 (s, 1H), 7.62 (d, J=8.4 Hz, 2H), 7.56 (d, J=8.4 Hz, 2H), 7.34 (s, 1H), 4.58 (s, 2H), 3.94 (s, 3H), 3.78 (s, 3H), 1.70-1.61 (m, 1H), 1.33-1.27 (m, 2H), 0.94-0.87 (m, 2H). ¹⁹F NMR (376 MHz, Chloroform-d) δ −62.60, −66.61.

### Example 118. 2-(4-cyclopropyl-6-isopropoxy-pyrimidin-5-yl)-7-[[4-[1-methyl-4-(trifluoromethyl)imidazol-2-yl]phenyl]methyl]-5H-pyrrolo[3,2-d]pyrimidine Compound 118

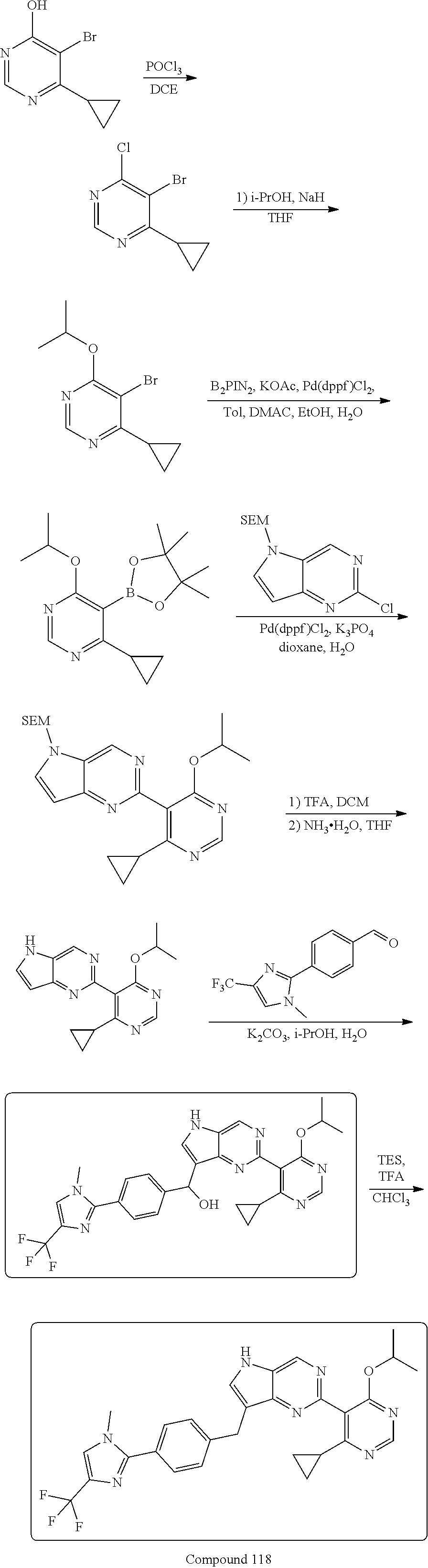

### 1.1 5-bromo-4-chloro-6-cyclopropyl pyrimidine

To a stirred solution of 5-bromo-6-cyclopropyl-pyrimidin-4-ol (2 g, 9.30 mmol) in 1,2-dichloroethane (36 mL) was added phosphorus oxychloride (6 mL) at 25° C. under nitrogen atmosphere. The resulting mixture was stirred for 6 hours at 80° C. under nitrogen atmosphere then concentrated under reduced pressure. The residue was purified by silica gel column chromatography, eluted with 30% ethyl acetate in petroleum ether to afford 5-bromo-4-chloro-6-cyclopropyl-pyrimidine (2.05 g, 8.78 mmol, 94% yield) as a white solid. MS: m/z=232.90, 234.90 [M+H]⁺.

### 1.2 5-bromo-4-cyclopropyl-6-isopropoxy-pyrimidine

To a stirred solution of anhydrous propan-2-ol (1.47 g, 24.41 mmol, 1.87 mL) in anhydrous THF (20.00 mL) was added sodium hydride (561 mg, 24.41 mmol, 60% dispersion in mineral oil) at 0° C. under nitrogen atmosphere. The resulting mixture was stirred for 30 min at 0° C. under nitrogen atmosphere. The mixture was added 5-bromo-4-chloro-6-cyclopropyl-pyrimidine (1.9 g, 8.14 mmol) at 0° C. under nitrogen atmosphere. The resulting mixture was stirred for 1 hour at 25° C. under nitrogen atmosphere. The reaction was quenched by the addition of saturated ammonia chloride aqueous solution (200 mL) at 0° C. The resulted mixture was extracted with ethyl acetate (3×100 mL). The combined organic layers were washed with brine (2×25 mL), dried over anhydrous sodium sulfate. After filtration, the filtrate was concentrated under reduced pressure. The residue was purified by silica gel column chromatography, eluted with 1%-10% methanol in dichloromethane to afford 5-bromo-4-cyclopropyl-6-isopropoxy-pyrimidine (1.7 g, 6.61 mmol, 81% yield) as a yellow oil. MS: m/z=256.95, 258.95 [M+H]⁺. ¹H NMR (400 MHz, Chloroform-d) δ 8.42 (s, 1H), 5.51-5.36 (m, 1H), 2.61-2.41 (m, 1H), 1.42 (d, J=6.4 Hz, 6H), 1.21-1.19 (m, 2H), 1.17-1.04 (m, 2H).

### 1.3 4-cyclopropyl-6-isopropoxy-5-(4,4,5,5-tetramethyl-1,3,2-dioxaborolan-2-yl)pyrimidine

To a stirred solution of 5-bromo-4-cyclopropyl-6-isopropoxy-pyrimidine (500 mg, 1.94 mmol) and 4,4,5,5-tetramethyl-2-(4,4,5,5-tetramethyl-1,3,2-dioxaborolan-2-yl)-1,3,2-dioxaborolane (988 mg, 3.89 mmol) in toluene (1.8 mL), DMA (1.2 mL), ethanol (1.2 mL) and water (0.6 mL) were added potassium phosphate (826 mg, 3.89 mmol) and 1,1′-Bis(diphenylphosphino)ferrocene-palladium (II) dichloride dichloromethane complex (159 mg, 194 μmol) at 25° C. under nitrogen atmosphere. The resulting mixture was stirred for 24 hours at 90° C. under nitrogen atmosphere. The resulting mixture was diluted with ethyl acetate (100 mL). The mixture was washed with brine (3×50 mL). The combined organic layers were dried over anhydrous sodium sulfate. After filtration, the filtrate was concentrated under reduced pressure to give 4-cyclopropyl-6-isopropoxy-5-(4,4,5,5-tetramethyl-1,3,2-dioxaborolan-2-yl)pyrimidine (591.52 mg, crude) as a black solid. MS: m/z=305.15 [M+H]⁺.

### 1.4 2-[[2-(4-cyclopropyl-6-isopropoxy-yl)pyrimidin-5-yl)pyrrolo[3,2-d]pyrimidin-5-yl]methoxy]ethyl-trimethyl-silane

To a stirred solution of 4-cyclopropyl-6-isopropoxy-5-(4,4,5,5-tetramethyl-1,3,2-dioxaborolan-2-yl)pyrimidine (500 mg, crude) and 2-[(2-chloropyrrolo[3,2-d]pyrimidin-5-yl)methoxy]ethyl-trimethyl-silane (467 mg, 1.64 mmol) in 1,4-dioxane (5 mL) and water (1 mL) were added potassium phosphate (698 mg, 3.29 mmol) and 1,1′-Bis(diphenylphosphino)ferrocene-palladium(II)dichloride dichloromethane complex (134 mg, 164 μmol) at 25° C. under nitrogen atmosphere. The resulting mixture was stirred for 16 hours at 100° C. under nitrogen atmosphere. The resulted mixture was filtered and the filter cake was washed with ethyl acetate (3×3 mL). The filtrate was concentrated under reduced pressure. The residue was purified by silica gel column chromatography, eluted with 1%-50% ethyl acetate in petroleum ether to afford 2-[[2-(4-cyclopropyl-6-isopropoxy-pyrimidin-5-yl)pyrrolo[3,2-d]pyrimidin-5-yl]methoxy]ethyl-trimethyl-silane (260 mg, 610.90 μmol, 37% yield) as a brown solid. MS: m/z=426.10 [M+H]⁺. ¹H NMR (400 MHz, Chloroform-d) δ 9.15 (s, 1H), 8.68 (s, 1H), 7.67 (d, J=3.2 Hz, 1H), 6.82 (d, J=3.2 Hz, 1H), 5.62 (s, 2H), 5.44-5.32 (m, 1H), 3.77-3.37 (m, 2H), 1.76-1.62 (m, 1H), 1.28-1.23 (m, 8H), 1.05-0.82 (m, 4H), 0.00 (s, 9H).

### 1.5 2-(4-cyclopropyl-6-isopropoxy-pyrimidin-5-yl)-5H-pyrrolo[3,2-d]pyrimidine

To a stirred solution of 2-[[2-(4-cyclopropyl-6-isopropoxy-pyrimidin-5-yl)pyrrolo[3,2-d]pyrimidin-5-yl]methoxy]ethyl-trimethyl-silane (200 mg, 470 μmol) in DCM (1 mL) was added 2,2,2-trifluoroacetic acid (1.48 g, 12.98 mmol, 1 mL) at 25° C. The resulting mixture was stirred for 1 hour at 25° C. The resulting mixture was concentrated under reduced pressure. The residue was dissolved in tetrahydrofuran (1 mL) and ammonium hydroxide (1 mL) was added at 25° C. The resulting mixture was stirred for 1 hour at 25° C. then concentrated under reduced pressure. The residue was purified by silica gel column chromatography, eluted with 1%-20% methanol in dichloromethane to afford 2-(4-cyclopropyl-6-isopropoxy-pyrimidin-5-yl)-5H-pyrrolo[3,2-d]pyrimidine (100 mg, 338.59 μmol, 72% yield) as a brown solid. MS: m/z=296.10 [M+H]⁺. ¹H NMR (400 MHz, Chloroform-d) δ 9.29 (s, 1H), 8.65 (s, 1H), 7.83 (s, 1H), 6.89 (d, J=2.8 Hz, 1H), 5.53-5.36 (m, 1H), 1.87-1.64 (m, 1H), 1.27 (s, 6H), 1.27-1.25 (m, 2H), 0.92-0.88 (m, 2H), 0.02 (s, 2H).

### 1.6 [2-(4-cyclopropyl-6-isopropoxy-pyrimidin-5-yl)-5H-pyrrolo[3,2-d]pyrimidin-7-yl]-[4-[1-methyl-4-(trifluoromethyl)imidazol-2-yl]phenyl]methanol

To a stirred solution of 2-(4-cyclopropyl-6-isopropoxy-pyrimidin-5-yl)-5H-pyrrolo[3,2-d]pyrimidine (100 mg, 338.59 μmol) and 4-[1-methyl-4-(trifluoromethyl)imidazol-2-yl]benzaldehyde (86 mg, 339 μmol) in water (1 mL) and propan-2-ol (1 mL) was added potassium carbonate (94 mg, 677 μmol) at 25° C. The resulting mixture was stirred for 24 hours at 50° C. under nitrogen atmosphere then concentrated under reduced pressure. The residue was purified by Prep-TLC (50% ethyl acetate in petroleum ether) to afford crude product. The obtained product was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 60% B in 20 min; 60% B to 60% B in 2 min, 60% B to 95% B in 26 min, Detector: UV 254 & 220 nm; RT: 25.5 min. The collected fractions were combined and lyophilized to give [2-(4-cyclopropyl-6-isopropoxy-pyrimidin-5-yl)-5H-pyrrolo[3,2-d]pyrimidin-7-yl]-[4-[1-methyl-4-(trifluoromethyl)imidazol-2-yl]phenyl]methanol (16 mg, 29.11 μmol, 8% yield) as an off-white solid. MS: m/z=550.25 [M+H]⁺. ¹H NMR (400 MHz, DMSO-d₆) δ 11.82 (s, 1H), 8.99 (s, 1H), 8.62 (s, 1H), 7.90 (s, 1H), 7.79 (d, J=2.8 Hz, 1H), 7.62 (s, 4H), 6.20 (d, J=4.8 Hz, 1H), 5.91 (d, J=4.8 Hz, 1H), 5.33-5.29 (m, 1H), 3.75 (s, 3H), 1.60-1.56 (m, 1H), 1.16-1.13 (m, 6H), 1.05-0.97 (m, 2H), 0.83-0.77 (m, 21H). ¹⁹F NMR (376 MHz, DMSO-d₆) δ −60.83.

### 1.7 2-(4-cyclopropyl-6-isopropoxy-pyrimidin-5-yl)-7-[[4-[1-methyl-4-(trifluoromethyl)imidazol-2-yl]phenyl]methyl]-5H-pyrrolo[3,2-d]pyrimidine

To a stirred solution of [2-(4-cyclopropyl-6-isopropoxy-pyrimidin-5-yl)-5H-pyrrolo[3,2-d]pyrimidin-7-yl]-[4-[1-methyl-4-(trifluoromethyl)imidazol-2-yl]phenyl]methanol (16 mg, 29 μmol) in DCM (2 mL) and triethylsilane (2 mL) was added 2,2,2-trifluoroacetic acid (1 mL) at 25° C. under nitrogen atmosphere. The resulting mixture was stirred for 2 hours at 25° C. under nitrogen atmosphere then concentrated under reduced pressure. The residue was purified by Prep-TLC (10% methanol in dichloromethane) to give crude product. The obtained product was purified by RP-Flash with the following conditions: Column: C18 spherical Column, 20-35 um, 100A, 25 g; Mobile Phase A: Water (5 mM aq. NH₄HCO₃), Mobile Phase B: MeCN; Flow rate: 35 mL/min; Gradient: 5% B to 5% B in 5 min, 5% B to 48% B in 17 min; 48% B to 48% B in 3 min, 48% B to 95% B in 23 min, Detector: UV 254 & 220 nm; RT: 23 min. The collected fractions were combined and lyophilized to give 2-(4-cyclopropyl-6-isopropoxy-pyrimidin-5-yl)-7-[[4-[1-methyl-4-(trifluoromethyl)imidazol-2-yl]phenyl]methyl]-5H-pyrrolo[3,2-d]pyrimidine (11 mg, 20.62 μmol, 70% yield) as a white solid. MS: m/z=534.2 [M+H]⁺. ¹H NMR (400 MHz, Chloroform-d) δ 9.40 (s, 1H), 8.66 (s, 1H), 7.65-7.54 (m, 3H), 7.44 (d, J=8.0 Hz, 2H), 7.32 (s, 1H), 5.46-5.40 (m, 1H), 4.32 (s, 2H), 3.77 (s, 3H), 1.83-1.79 (m, 1H), 1.30-1.26 (m, 8H), 0.90-0.86 (m, 2H). ¹⁹F NMR (376 MHz, Chloroform-d) δ −62.71.

### Example 119. 2-[4-(cyclopropyl)-6-cyclopropyl-pyrimidin-5-yl]-7-[[4-[1-methyl-4-(trifluoromethyl)imidazol-2-yl]phenyl]methyl]-5H-pyrrolo[3,2-d]pyrimidine Compound 155

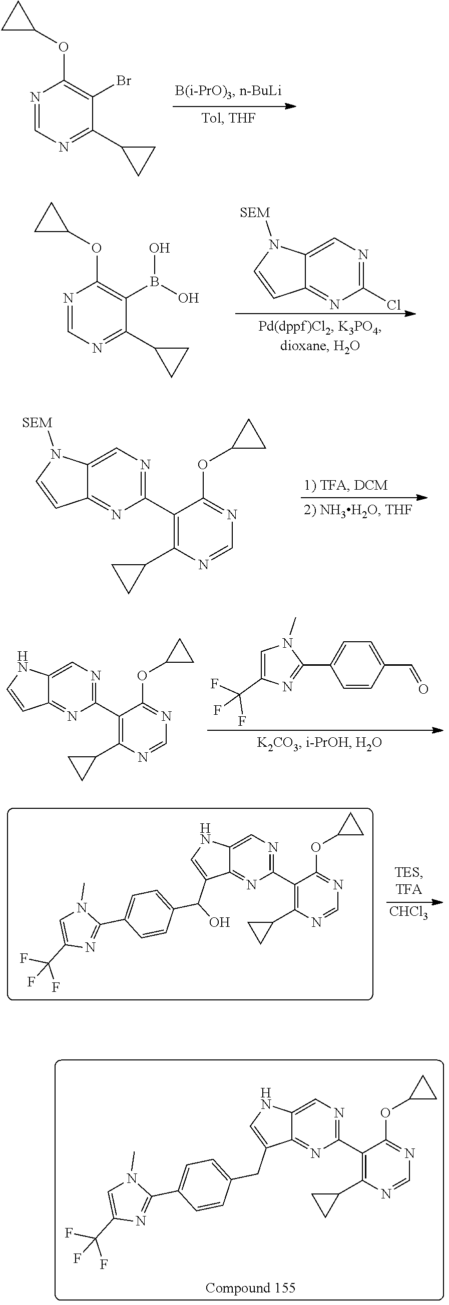

### 1.1 [4-(cyclopropyl)-6-cyclopropyl-pyrimidin-5-yl]boronic Acid

To a solution of 5-bromo-4-(cyclopropyl)-6-cyclopropyl-pyrimidine (1.5 g, 5.88 mmol) and triisopropyl borate (2.21 g, 11.76 mmol, 2.71 mL) in toluene (16 mL) and THF (4 mL) was added dropwise n-butyllithium solution (2.5 M in hexane, 4.70 mL, 11.76 mmol) at −70° C. under nitrogen atmosphere. The reaction mixture was stirred at −70° C. for 1 hours then quenched with 1 N Hydrogen chloride aqueous solution (20 mL). The resulting mixture was stirred at room temperature for 30 minutes and 90% desired product could be detected by LCMS. After concentration, the residue was purified by RP-Flash with the following conditions: Column: Spherical C18, 20-40 μm, 330 g; Mobile Phase A: Water (5 mM aq. HCl), Mobile Phase B: ACN; Flow rate: 80 mL/min; Gradient (B %): 0% hold 5 min, 0%-20% within 20 min, 20% hold 5 min; 20%-95% within 10 min, 95% hold 5 min; Detector: UV 254 & 210 nm; RT: 27 min. The collected fractions were combined and concentrated under reduced pressure to afford [4-(cyclopropyl)-6-cyclopropyl-pyrimidin-5-yl]boronic acid (1.1 g, 5.00 mmol, 85% yield) as an off-white solid. MS: m/z=221.05 [M+H]⁺. ¹H NMR (400 MHz, Methanol-d₄) δ 8.69 (s, 1H), 4.48-4.42 (m, 1H), 1.95-1.87 (m, 1H), 1.20-1.11 (m, 4H), 0.93-0.75 (m, 4H).

### 1.2 2-[[2-[4-(cyclopropyl)-6-cyclopropyl-pyrimidin-5-yl]pyrrolo[3,2-d]pyrimidin-5-yl]methoxy]ethyl-trimethyl-silane

To a solution of [4-(cyclopropyl)-6-cyclopropyl-pyrimidin-5-yl]boronic acid (560 mg, 2.55 mmol) in 1,4-dioxane (10 mL) and water (2 mL) was added 2-[(2-chloropyrrolo[3,2-d]pyrimidin-5-yl)methoxy]ethyl-trimethyl-silane (657 mg, 2.31 mmol), 1,1′-Bis(diphenylphosphino)ferrocene-palladium (II) dichloride dichloromethane complex (189 mg, 231 μmol) and potassium phosphate (982 mg, 4.63 mmol) and then the solution was stirred for 16 hours at 100° C. under nitrogen atmosphere. The reaction solution was diluted with ethyl acetate (300 mL), washed with brine (3×50 mL), dried over anhydrous sodium sulfate. After filtration, the filtrate was concentrated under reduced pressure. The residue was purified by silica gel column chromatography, eluted with 30% ethyl acetate in petroleum ether to give 2-[[2-[4-(cyclopropyl)-6-cyclopropyl-pyrimidin-5-yl]pyrrolo[3,2-d]pyrimidin-5-yl]methoxy]ethyl-trimethyl-silane (380 mg, 897.11 μmol, 38% yield) as a yellow solid. MS: m/z=424.15 [M+H]. ¹H NMR (400 MHz, Chloroform-d) δ 9.14 (s, 1H), 8.76 (s, 1H), 7.67 (d, J=3.2 Hz, 1H), 6.82 (d, J=3.2 Hz, 1H), 5.62 (s, 2H), 4.38-4.32 (m, 11H), 3.62-3.56 (m, 2H), 1.72-1.66 (m, 1H), 1.30-1.28 (m, 4H), 1.00-0.97 (m, 2H), 0.76-0.68 (m, 4H), 0.02 (s, 9H).

### 1.3 2-[4-(cyclopropyl)-6-cyclopropyl-pyrimidin-5-yl]-5H-pyrrolo[3,2-d]pyrimidine

To a solution of 2-[[2-[4-(cyclopropyl)-6-cyclopropyl-pyrimidin-5-yl]pyrrolo[3,2-d]pyrimidin-5-yl]methoxy]ethyl-trimethyl-silane (390 mg, 921 μmol) in DCM (4 mL) was added TFA (4 mL). After stirring at 25° C. for 1 hour, the reaction solution was concentrated under reduced pressure. The residue was added ammonium hydroxide (4 mL) and THF (4 mL) with stirring at 25° C. The mixture was stirred at 25° C. for 1 hour. The resulting solution was detected by LCMS, major desired product. The resulted solution was concentrated under reduced pressure. The residue was purified by silica gel column chromatography, eluted with 30% ethyl acetate in petroleum ether to give 2-[4-(cyclopropyl)-6-cyclopropyl-pyrimidin-5-yl]-5H-pyrrolo[3,2-d]pyrimidine (260 mg, 886.40 μmol, 96% yield) as a yellow solid. MS: m/z=294.05 [M+H]⁺. ¹H NMR (400 MHz, Methanol-d₄) δ 9.01 (s, 1H), 8.67 (s, 1H), 7.97 (d, J=3.2 Hz, 1H), 6.72 (d, J=3.2 Hz, 1H), 4.39-4.33 (m, 1H), 1.61-1.54 (m, 1H), 1.21-1.12 (m, 2H), 0.95-0.89 (m, 2H), 0.76-0.70 (m, 2H), 0.63-0.55 (m, 2H).

### 1.4 [2-[4-(cyclopropyl)-6-cyclopropyl-pyrimidin-5-yl]-5H-pyrrolo[3,2-d]pyrimidin-7-yl]-[4-[1-methyl-4-(trifluoromethyl)imidazol-2-yl]phenyl]methanol

To a solution of 2-[4-(cyclopropyl)-6-cyclopropyl-pyrimidin-5-yl]-5H-pyrrolo[3,2-d]pyrimidine (200 mg, 682 μmol) in isopropyl alcohol (3 mL) and water (3 mL) was added 4-[1-methyl-4-(trifluoromethyl)imidazol-2-yl]benzaldehyde (173 mg, 682 μmol) and potassium carbonate (188 mg, 1.36 mmol) and then the solution was stirred for 16 hours at 50° C. under nitrogen atmosphere. The resulting mixture was diluted with ethyl acetate (150 mL). The organic layers were washed with brine (3×50 mL), dried over anhydrous sodium sulfate. After filtration, the filtrate was concentrated under reduced pressure. The residue was purified by Prep-TLC, eluted with 10% methanol in dichloromethane to give 100 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 56% B in 11 min, 56% B to 56% B in 2 min, 56% B to 95% B in 9 min. 95% B to 95% B in 5 min; Detector: UV 254 & 210 nm; RT: 18 min. The collected fractions were combined, concentrated and then lyophilized to give [2-[4-(cyclopropyl)-6-cyclopropyl-pyrimidin-5-yl]-5H-pyrrolo[3,2-d]pyrimidin-7-yl]-[4-[1-methyl-4-(trifluoromethyl)imidazol-2-yl]phenyl]methanol (87.6 mg, 159.99 μmol, 23% yield) as an off-white solid. MS: m/z=548.20 [M+H]⁺. ¹H NMR (400 MHz. Methanol-d₄) δ 8.97 (s, 1H), 8.66 (s, 1H), 7.80 (s, 1H), 7.74-7.68 (m, 3H), 7.64 (d, J=8.4 Hz, 2H), 6.41 (s, 1H), 4.39-4.33 (m, 1H), 3.79 (s, 3H), 1.65-1.57 (m, 1H), 1.19-1.13 (m, 2H), 0.91-0.85 (m, 2H), 0.75-0.68 (m, 2H), 0.61-0.56 (m, 2H). ¹⁹F NMR (376 MHz, Methanol-d₄) δ −63.94.

### 1.5 2-[4-(cyclopropyl)-6-cyclopropyl-pyrimidin-5-yl]-7-[[4-[1-methyl-4-(trifluoromethyl)imidazol-2-yl]phenyl]methyl]-5H-pyrrolo[3,2-d]pyrimidine

To a solution of [2-[4-(cyclopropyl)-6-cyclopropyl-pyrimidin-5-yl]-5H-pyrrolo[3,2-d]pyrimidin-7-yl]-[4-[1-methyl-4-(trifluoromethyl)imidazol-2-yl]phenyl]methanol (64 mg, 117 μmol) in chloroform (1 mL) and triethylsilane (I mL) was added TFA (0.5 mL) at 20° C., then the solution was stirred at room temperature for 16 hours. The resulted solution was detected by LCMS, major desired product. The resulted solution was concentrated under reduced pressure. The residue was co-evaporated with toluene (three times, 5 mL each). The residue was purified by Prep-TLC, eluted with 10% methanol in dichloromethane to give 50 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 65 mL/min; Gradient: 5% B to 5% B in 5 min, 5% B to 51% B in 10 min, 51% B to 51% B in 2 min, 51% B to 95% B in 10 min; Detector: UV 254 & 210 nm; RT: 17 min. The collected fractions were combined, concentrated and then lyophilized to give 2-[4-(cyclopropyl)-6-cyclopropyl-pyrimidin-5-yl]-7-[[4-[1-methyl-4-(trifluoromethyl)imidazol-2-yl]phenyl]methyl]-5H-pyrrolo[3,2-d]pyrimidine (30.7 mg, 57.76 μmol, 49% yield) as an off-white solid. MS: m/z=532.20 [M+H]⁺. ¹H NMR (400 MHz, Methanol-d₄) δ 8.96 (s, 1H), 8.66 (s, 1H), 7.76 (s, 1H), 7.67 (s, 1H), 7.57 (d, J=8.0 Hz, 2H), 7.51 (d, J=8.0 Hz, 2H), 4.40-4.34 (m, 1H), 4.29 (s, 2H), 3.77 (s, 3H), 1.64-1.56 (m, 1H), 1.19-1.14 (m, 2H), 0.92-0.86 (m, 2H), 0.76-0.69 (m, 2H), 0.61-0.56 (m, 2H). ¹⁹F NMR (376 MHz, Methanol-d₄) δ −63.94.

### Example 120. 2-(4,6-dicyclopropylpyrimidin-5-yl)-7-[[4-[1-methyl-4-(trifluoromethyl)imidazol-2-yl]phenyl]methyl]-5H-pyrrolo[3,2-d]pyrimidine Compound 193

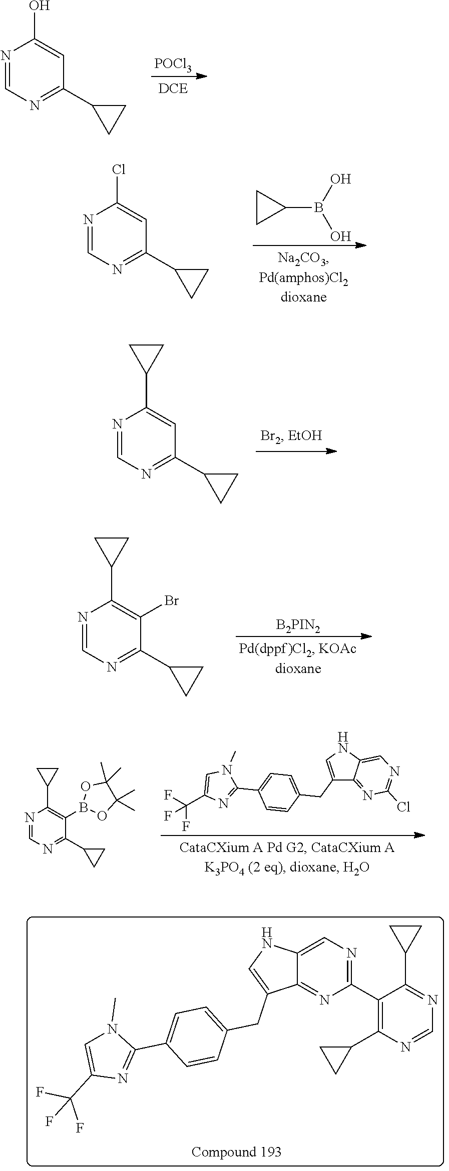

### 1.1 4-chloro-6-cyclopropyl-pyrimidine

To a stirred solution of 6-cyclopropylpyrimidin-4-ol (2.2 g, 16.16 mmol) in DCM (30 mL) was added Phosphorus oxychloride (5 mL) at room temperature. The mixture was stirred at 80° C. for 2 hours, 58% desired product could be detected by LCMS. The resulting mixture was concentrated under reduced pressure. The residue was purified by silica gel column chromatography, eluted with 50% ethyl acetate in petroleum ether to give 4-chloro-6-cyclopropyl-pyrimidine (2 g, 12.94 mmol, 80% yield) as a yellow solid. MS: m/z=155.00 [M+H]⁺. ¹H NMR (400 MHz, Chloroform-d) δ 8.78 (s, 1H), 7.24 (s, 1H), 2.04-1.97 (m, 1H), 1.25-1.15 (m, 4H).

### 1.2 4,6-dicyclopropylpyrimidine

To a stirred solution of 4-chloro-6-cyclopropyl-pyrimidine (1.5 g, 9.70 mmol) in 1,4-dioxane (30 mL) and water (15 mL) were added bis(di-tert-butyl(4-dimethylaminophenyl)phosphine)dichloropalladium (II) (687 mg, 970 μmol) and sodium carbonate (3.09 g, 29.11 mmol) at room temperature. The resulting mixture was stirred at 130° C. for 2 hours under nitrogen atmosphere, 70% desired product could be detected by LCMS. The resulting mixture was concentrated under reduced pressure. The residue was purified by silica gel column chromatography, eluted with ethyl acetate in petroleum ether (0%-13%) to afford 4,6-dicyclopropylpyrimidine (800 mg, 4.99 mmol, 51% yield) as a yellow oil. MS: m/z=161.10 [M+H]⁺. ¹H NMR (400 MHz, Chloroform-d) δ 8.83 (s, 1H), 7.03 (s, 1H), 2.08-2.00 (m, 2H), 1.20-1.17 (m, 8H).

### 1.3 5-bromo-4,6-dicyclopropyl-pyrimidine

To a stirred solution of 4,6-dicyclopropylpyrimidine (600 mg, 3.74 mmol) in EtOH (1 mL) was added molecular bromine (598 mg, 3.74 mmol) at −10° C. After the resulting mixture was stirred at −10° C. for 3 hours, ammonia in MeOH solution (2 N, 0.1 mL) was added at 0° C. The resulting mixture was concentrated under reduced pressure. The residue was purified by silica gel column chromatography, eluted with ethyl acetate in petroleum ether (0%-13%) to afford 5-bromo-4,6-dicyclopropyl-pyrimidine (250 mg, 1.05 mmol, 28% yield) as a yellow oil. MS: m/z=239.00, 241.00 [M+H]⁺. ¹H NMR (400 MHz, Chloroform-d) δ 8.67 (s, 11H), 2.64-2.57 (m, 2H), 1.29-1.27 (m, 4H), 1.21-1.16 (m, 4H).

### 1.4 4,6-dicyclopropyl-5-(4,4,5,5-tetramethyl-1,3,2-dioxaborolan-2-yl)pyrimidine

To a solution of 5-bromo-4,6-dicyclopropyl-pyrimidine (30 mg, 125.46 μmol) and 4,4,5,5-tetramethyl-2-(4,4,5,5-tetramethyl-1,3,2-dioxaborolan-2-yl)-1,3,2-dioxaborolane (48 mg, 188 μmol) in 1,4-dioxane (1 mL) was added potassium acetate (25 mg, 251 μmol) and 1,1′-bis(diphenylphosphino)ferrocene-palladium(II)dichloride (10 mg, 12 μmol) at 25° C. The resulting mixture was stirred at 90° C. for 5 hours under nitrogen atmosphere then concentrated under reduced pressure. The residue was purified by Prep-TLC, eluted with 40% ethyl acetate in petroleum ether to give 4,6-dicyclopropyl-5-(4,4,5,5-tetramethyl-1,3,2-dioxaborolan-2-yl)pyrimidine (20 mg, 69.89 μmol, 55.70% yield) as a brown oil. MS: m/z=287.10 [M+H]⁺. ¹H NMR (400 MHz, Chloroform-d) δ 9.24 (s, 1H), 8.85 (s, 1H), 7.56 (d, J=8.0 Hz, 2H), 7.40 (d, J=8.0 Hz, 2H), 7.34 (s, 1H), 4.28 (s, 2H), 3.89 (s, 3H), 3.77 (s, 3H), 1.12 (s, 9H). ¹H NMR (400 MHz, Chloroform-d) δ 8.84 (s, 1H), 2.37-2.33 (s, 2H), 1.44 (s, 12H), 1.28-1.24 (s, 4H), 1.18-1.14 (m, 4H).

### 1.5 2-(4,6-dicyclopropylpyrimidin-5-yl)-7-[[4-[1-methyl-4-(trifluoromethyl)imidazol-2-yl]phenyl]methyl]-5H-pyrrolo[3,2-d]pyrimidine

To a stirred mixture of 2-chloro-7-[[4-[1-methyl-4-(trifluoromethyl)imidazol-2-yl]phenyl]methyl]-5H-pyrrolo[3,2-<]pyrimidine (18 mg, 47 μmol) and 4,6-dicyclopropyl-5-(4,4,5,5-tetramethyl-1,3,2-dioxaborolan-2-yl)pyrimidine (20 mg, 69 μmol) in 1,4-dioxane (1 mL) and water (0.2 mL) were added chloro[(di(1-adamantyl)-N-butylphosphine)-2-(2-aminobiphenyl)]palladium (II) (3.12 mg, 4.66 μmol), butyldi-1-adamantylphosphine (1.67 mg, 4.66 μmol) and potassium phosphate (20 mg, 93 μmol) under nitrogen atmosphere at 25° C. The resulting mixture was stirred at 90° C. for 16 hours. The reaction was cooled down to room temperature, then diluted with water (100 mL), extracted with dichloromethane (3×100 mL). The combined organic layers were washed with brine (100 mL), dried over anhydrous sodium sulfate. After filtration, the filtrate was concentrated under reduced pressure. The residue was purified by Prep-TLC, eluted with 10% MeOH in DCM to give 10 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 50% B in 13 min, 50% B to 50% B in 2 min, 50% B to 95% B in 5 min; Detector: UV 254& 210 nm; RT: 19 min. The collected fractions were combined, concentrated and then lyophilized to give 2-(4,6-dicyclopropylpyrimidin-5-yl)-7-[[4-[1-methyl-4-(trifluoromethyl)imidazol-2-yl]phenyl]methyl]-5H-pyrrolo[3,2-d]pyrimidine (5 mg, 9.70 μmol, 21% yield) as an off-white solid. MS: m/z=516.30 [M+H]⁺. ¹H NMR (400 MHz, Chloroform-d) δ 10.64 (s, 1H), 9.09 (s, 1H), 8.85 (s, 1H), 7.54 (d, J=8.0 Hz, 2H), 7.43 (d, J=8.0 Hz, 2H), 7.38 (s, 1H), 7.30 (s, 1H), 4.30 (s, 2H), 3.79 (s, 3H), 1.67-1.60 (m, 2H), 1.30-1.19 (m, 4H), 0.88-0.84 (m, 4H). ¹⁹F NMR (376 MHz, Chloroform-d) δ −62.46.

### Example 121. 5-(4,6-dicyclopropylpyrimidin-5-yl)-3-[[4-[1-methyl-4-(trifluoromethyl)imidazol-2-yl]phenyl]methyl]-1H-pyrazolo[4,3-d]pyrimidine Compound 183

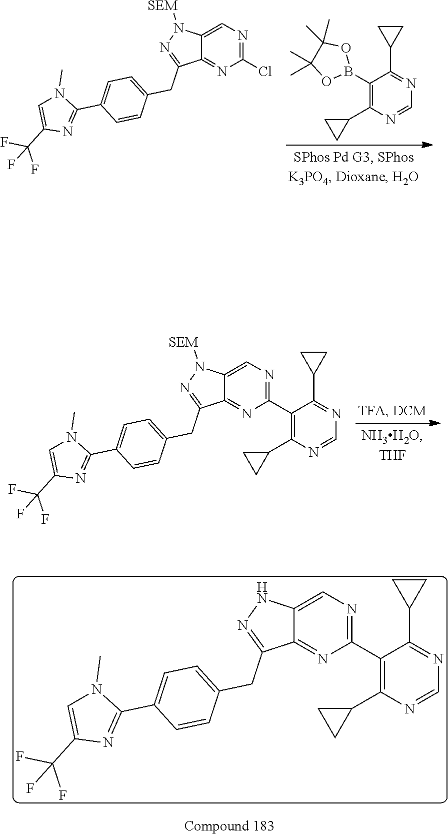

### 1.1 2-[[5-(4,6-dicyclopropylpyrimidin-5-yl)-3-[[4-f -methyl-4-(trifluoromethyl)imidazol-2-yl]phenyl]methyl pyrazolo[4,3-d]pyrimidin-1-yl]methoxy]ethyl-trimethyl-silane

To a stirred mixture of 2-[[5-chloro-3-[[4-[1-methyl-4-(trifluoromethyl)imidazol-2-yl]phenyl]methyl]pyrazolo[4,3-d]pyrimidin-1-yl]methoxy]ethyl-trimethyl-silane (200 mg, 382.39 μmol) and 4,6-dicyclopropyl-5-(4,4,5,5-tetramethyl-1,3,2-dioxaborolan-2-yl)pyrimidine (164.15 mg, 573.59 μmol) in 1,4-dioxane (4 mL) and water (0.8 mL) were added (2-Dicyclohexylphosphino-2′,6′-dimethoxybiphenyl)[2-(2′-amino-1,1′-biphenyl)]palladium(II) methanesulfonate (30 mg, 38 μmol) and 2-Dicyclohexylphosphino-2′,6′-dimethoxybiphenyl (16 mg, 38 μmol) and potassium phosphate (162 mg, 765 μmol) under nitrogen atmosphere at 25° C. The resulting mixture was stirred at 100° C. for 16 hours. The reaction was cooled down to room temperature, then quenched with water (50 mL), extracted with dichloromethane (3×50 mL). The combined organic layers were washed with brine (100 mL), dried over anhydrous sodium sulfate. After filtration, the filtrate was concentrated under reduced pressure. The residue was purified by Prep-TLC, eluted with 10% methanol in dichloromethane to give 2-[[5-(4,6-dicyclopropylpyrimidin-5-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane (90 mg, 139.15 μmol, 37% yield) as an off-white solid. MS: m/z=647.35 [M+H]⁺. ¹H NMR (400 MHz, Chloroform-d) δ 9.40 (s, 1H), 9.01 (s, 1H), 7.56 (s, 4H), 7.31 (s, 1H), 5.85 (s, 2H), 4.53 (s, 2H), 3.76 (s, 3H), 3.73-3.65 (m, 2H), 1.71-1.63 (m, 2H), 1.54-1.50 (s, 4H), 1.10-1.06 (m, 4H), 1.02-0.94 (m, 2H), −0.01 (s, 9H).

### 1.2 5-(4,6-dicyclopropylpyrimidin-5-yl)-3-[[4-[1-methyl-4-(trifluoromethyl)imidazol-2-yl]phenyl]methyl]-1H-pyrazolo[4,3-d]pyrimidine

To a solution of 2-[[5-(4,6-dicyclopropylpyrimidin-5-yl)-3-[[4-[1-methyl-4-(trifluoromethyl)imidazol-2-yl]phenyl]methyl]pyrazolo[4,3-d]pyrimidin-1-yl]methoxy]ethyl-trimethyl-silane (80 mg, 124 μmol) in dichloromethane (1 mL) was added trifluoroacetic acid (1 mL) at 25° C., and then stirred at this temperature for 2 hours. The reaction solution was concentrated under reduced pressure. The residue was dissolved in tetrahydrofuran (1 mL) and ammonium hydroxide (1 mL) was added at 0° C. The resulted mixture was stirred at 25° C. for 0.5 hour. The resulted solution was concentrated under reduced pressure. The residue was purified by Prep-TLC, eluted with 7% methanol in dichloromethane to give 30 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 63% B in 10 min, 63% B to 63% B in 2 min, 63% B to 95% B in 5 min; Detector: UV 254 & 210 nm; RT: 17 min. The collected fractions were combined, concentrated and then lyophilized to give 5-(4,6-dicyclopropylpyrimidin-5-yl)-3-[[4-[1-methyl-4-(trifluoromethyl)imidazol-2-yl]phenyl]methyl]-1H-pyrazolo[4,3-d]pyrimidine (18.7 mg, 36.20 μmol, 30% yield) as an off-white solid. MS: m/z=517.15 [M+H]⁺. ¹H NMR (400 MHz, Chloroform-d) δ 9.23 (s, 1H), 8.90 (s, 1H), 7.70 (d, J=8.0 Hz, 2H), 7.61 (d, J=8.0 Hz, 2H), 7.42 (s, 1H), 4.56 (s, 2H), 3.84 (s, 3H), 1.63-1.57 (m, 2H), 1.31-1.27 (s, 4H), 0.93-0.85 (m, 4H). ¹⁹F NMR (377 MHz, Chloroform-d) δ −62.17.

### Example 122, 2-(4-isopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine Compound 105

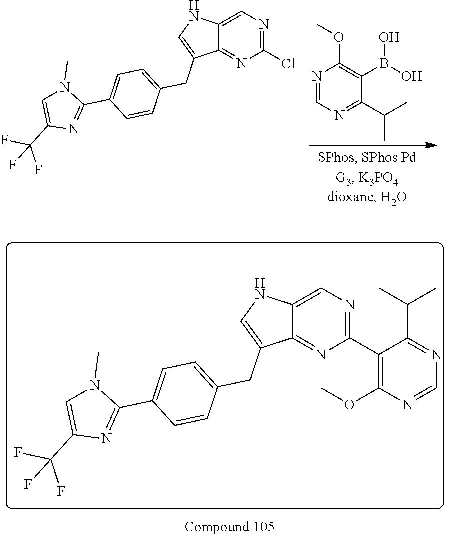

### 1.1 2-(4-isopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine

To a stirred mixture of 2-chloro-7-[[4-[1-methyl-4-(trifluoromethyl)imidazol-2-yl]phenyl]methyl]-5H-pyrrolo[3,2-d]pyrimidine (100 mg, 255 μmol) and (4-isopropyl-6-methoxy-pyrimidin-5-yl)boronic acid (150 mg, 766 μmol) in 1,4-dioxane (3 mL) and water (0.6 mL) were added 2-Dicyclohexylphosphino-2′,6′-dimethoxybiphenyl (21 mg, 51 μmol), (2-Dicyclohexylphosphino-2′,6′-dimethoxybiphenyl)[2-(2′-amino-1,1′-biphenyl)]palladium(II) methanesulfonate (40 mg, 51 μmol) and potassium phosphate (163 mg, 766 μmol) at 25° C. under nitrogen atmosphere. The resulting mixture was stirred at 100° C. for 16 hours. The reaction was cooled down to room temperature, then quenched with water (100 mL), extracted with dichloromethane (3×100 mL). The combined organic layers were washed with brine (100 mL), dried over anhydrous sodium sulfate. After filtration, the filtrate was concentrated under reduced pressure. The residue was purified by Prep-TLC, eluted with 10% methanol in dichloromethane to give 30 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 50% B in 13 min, 50% B to 50% B in 2 min, 50% B to 95% B in 5 min; Detector: UV 254&210 nm; RT: 19 min. The collected fractions were combined, concentrated and then lyophilized to give 2-(4-isopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (20.5 mg, 40.39 μmol, 16% yield) as an off-white solid. MS: m/z=508.30 [M+H]⁺. ¹H NMR (400 MHz, Chloroform-d) δ 10.43 (s, 1H), 9.07 (s, 1H), 8.85 (s, 1H), 7.55 (d, J=8.4 Hz, 2H), 7.42 (d, J=8.4 Hz, 2H), 7.36 (s, 1H), 7.33 (s, 1H), 4.27 (s, 2H), 3.95 (s, 3H), 3.78 (s, 3H), 2.84-2.77 (m, 1H), 1.21 (d, J=6.8 Hz, 6H). ¹⁹F NMR (377 MHz, Chloroform-d) δ −62.52.

### Example 123. 2-(4-tert-butyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine Compound 124

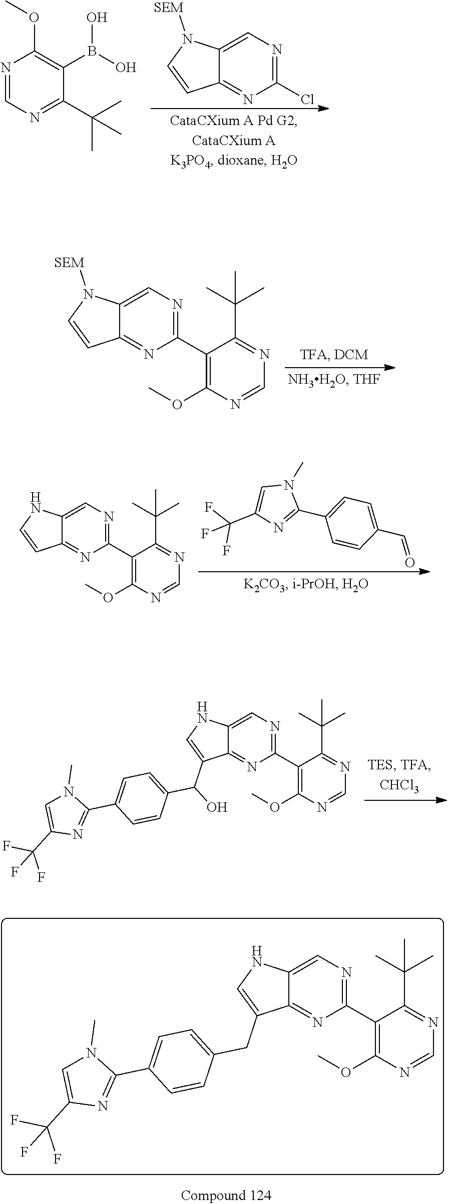

### 1.1 2-[[2-(4-tert-butyl-6-methoxy-pyrimidin-5-yl)pyrrolo[3,2-d]pyrimidin-5-yl]methoxy]ethyl-trimethyl-silane

To a stirred mixture of (4-tert-butyl-6-methoxy-pyrimidin-5-yl)boronic acid (300 mg, 1.43 mmol) and 2-[(2-chloropyrrolo[3,2-d]pyrimidin-5-yl)methoxy]ethyl-trimethyl-silane (203 mg, 714 μmol) in 1,4-dioxane (5 mL) and water (1 mL) were added chloro[(di(1-adamantyl)-N-butylphosphine)-2-(2-aminobiphenyl)]palladium(II) (48 mg, 71 μmol), butyldi-1-adamantylphosphine (25.5 mg, 71 μmol) and potassium phosphate (454 mg, 2.14 mmol) at 25° C. under nitrogen atmosphere. The resulting mixture was stirred at 90° C. for 16 hours. The reaction was cooled down to room temperature, 18% desired product could be detected by LCMS. The reaction was quenched with water (50 mL), extracted with dichloromethane (3×50 mL). The combined organic layers were washed with brine (50 mL), dried over anhydrous sodium sulfate. After filtration, the filtrate was concentrated under reduced pressure. The residue was purified by Prep-TLC, eluted with 10% methanol in dichloromethane to give 2-[[2-(4-tert-butyl-6-methoxy-pyrimidin-5-yl)pyrrolo[3,2-d]pyrimidin-5-yl]methoxy]ethyl-trimethyl-silane (40 mg, 96.71 μmol, 14% yield) as an off-white solid. MS: m/z=414.25 [M+H]⁺. ¹H NMR (400 MHz, Chloroform-d) δ 9.07 (s, 1H), 8.83 (s, 1H), 7.64 (d, J=3.2 Hz, 1H), 6.77 (d. J=3.2 Hz, 1H), 5.61 (s, 2H), 3.87 (s, 3H), 3.58 (t, J=8.0 Hz, 2H), 1.14 (s, 9H), 0.96 (t, J=8.0 Hz, 2H), −0.01 (s, 9H).

### 1.2 2-(4-tert-butyl-6-methoxy-pyrimidin-5-yl)-5H-pyrrolo[3,2-d]pyrimidine

To a solution of 2-[[2-(4-tert-butyl-6-methoxy-pyrimidin-5-yl)pyrrolo[3,2-d]pyrimidin-5-yl]methoxy]ethyl-trimethyl-silane (40 mg, 97 μmol) in dichloromethane (1 mL) was added trifluoroacetic acid (1 mL) at 25° C., and then stirred at this temperature for 2 hours. The reaction solution was concentrated under reduced pressure. The residue was dissolved in tetrahydrofuran (1 mL) and ammonium hydroxide (1 mL) was added at 0° C. The resulted mixture was stirred at 25° C. for 0.5 hour and then concentrated under reduced pressure. The residue was purified by Prep-TLC, eluted with 7% methanol in dichloromethane to give 2-(4-tert-butyl-6-methoxy-pyrimidin-5-yl)-5H-pyrrolo[3,2-d]pyrimidine (20 mg, 70.59 μmol, 73% yield) as an off-white solid. MS: m/z=284.10 [M+H]⁺.

### 1.3 [2-(4-tert-butyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol

To a solution of 2-(4-tert-butyl-6-methoxy-pyrimidin-5-yl)-5H-pyrrolo[3,2-d]pyrimidine (20 mg, 70 μmol) in isopropyl alcohol (1 mL) and water (1 mL) were added 4-[1-methyl-4-(trifluoromethyl)imidazol-2-yl]benzaldehyde (18 mg, 71 μmol) and potassium carbonate (20 mg, 141 μmol). The resulting mixture was stirred at 50° C. for 36 hours. The reaction solution was detected by TLC and LCMS. The resulted mixture was diluted water (10 mL), extracted with ethyl acetate (3×5 mL). The combined organic layers were washed with brine (5 mL), dried over anhydrous sodium sulfate. After filtration, the filtrate was concentrated under reduced pressure. The residue was purified by Prep-TLC, eluted with 10% methanol in dichloromethane to give [2-(4-tert-butyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol (10 mg, 18.60 μmol, 26% yield) as an off-white solid. MS: m/z=538.25 [M+H]⁺.

### 1.4 2-(4-tert-butyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine

To a solution of [2-(4-tert-butyl-6-methoxy-pyrimidin-5-yl)-5H-pyrrolo[3,2-d]pyrimidin-7-yl]-[4-[1-methyl-4-(trifluoromethyl)imidazol-2-yl]phenyl]methanol (10 mg, 18.6 μmol) in chloroform (1 mL) was added trifluoroacetic acid (0.5 mL) and triethylsilicane (1 mL) at 25° C., then was stirred at 25° C. for 24 hours. The resulted solution was detected by LCMS, major desired product. The resulted solution was concentrated under reduced pressure. The residue was purified by Prep-TLC, eluted with 10% methanol in dichloromethane to give 5 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 63% B in 10 min, 63% B to 63% B in 2 min, 63% B to 95% B in 5 min; Detector: UV 254 & 210 nm; RT: 17 min. The collected fractions were combined, concentrated and then lyophilized to give 2-(4-tert-butyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (3 mg, 5.75 μmol, 31% yield) as an off-white solid. MS: m/z=522.35 [M+H]⁺. ¹H NMR (400 MHz, Chloroform-d) δ 9.24 (s, 1H), 8.85 (s, 1H), 7.56 (d, J=8.0 Hz, 2H), 7.40 (d, J=8.0 Hz, 2H), 7.34 (s, 1H), 7.31 (s, 1H), 4.28 (s, 2H), 3.89 (s, 3H), 3.77 (s, 3H), 1.12 (s, 9H). ¹⁹F NMR (377 MHz, Chloroform-d) δ −62.63.

### Example 124. 1-cyclopropyl-5-(4-cyclopropyl-6-methoxy-pyrimidin-5-yl)-3-[[4-[1-methyl-4-trifluoromethyl)imidazol-2-yl]phenyl]methyl]pyrazolo[4,3-d]pyrimidine Compound 130

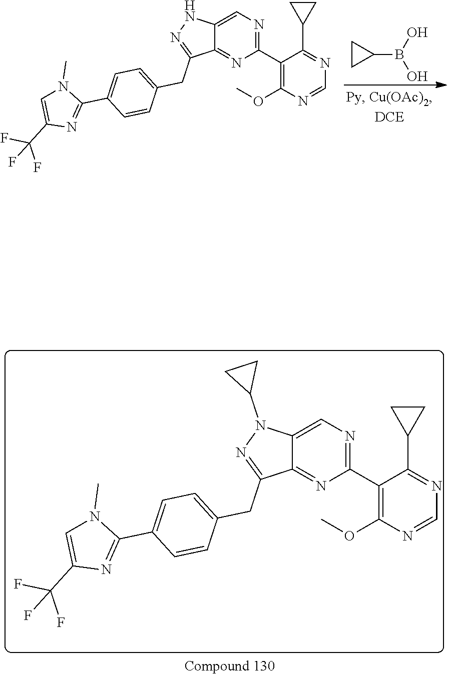

### 1.1 1-cyclopropyl-5-(4-cyclopropyl-6-methoxy-pyrimidin-5-yl)-3-[[4-[1-methyl-4-(trifluoromethyl)imidazol-2-yl]phenyl]methyl]pyrazolo[4,3-d]pyrimidine

To a solution of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (51 mg, 0.1 mmol) and cyclopropylboronic acid (26 mg, 300 μmol) in 1,2-dichloroethane (5 mL) were added pyridine (40 mg, 500 μmol, 40 μL) and cupric acetate monohydrate (40 mg, 200 μmol) at room temperature, then stirred at 60° C. for 16 hours. The reaction mixture was cooled down to room temperature. Major target product was detected by LCMS. The resulted mixture was concentrated under reduced pressure. The residue was purified by RP-Flash with the following conditions: Column: C18 gel column 40 g, 20-35 μm; Mobile Phase A: Water (5 mM aq. NH₄HCO₃), Mobile Phase B: MeCN; Gradient: 5% B hold 5 min, up to 61% B within 18 min, 61% B hold 5 min, up to 95% B within 5 min; Flow rate: 40 mL/min; Detector: UV 254 & 220 nm: RT: 26 min. The product-containing fractions were collected, concentrated and then lyophilized overnight to give I-cyclopropyl-5-(4-cyclopropyl-6-methoxy-pyrimidin-5-yl)-3-[[4-[1-methyl-4-(trifluoromethyl)imidazol-2-yl]phenyl]methyl]pyrazolo[4,3-d]pyrimidine (23.5 mg, 43.00 μmol, 43% yield) as a white solid. MS: m/z=547.15 [M+H]⁺. ¹H NMR (400 MHz, Methanol-d₄) δ 9.49 (s, 1H), 8.64 (s, 1H), 7.66 (s, 1H), 7.56 (s, 4H), 4.48 (s, 2H), 3.94-3.89 (m, 4H), 3.75 (s, 3H), 1.67-1.61 (m, 1H), 1.35-1.24 (m, 4H), 1.20-1.16 (m, 2H), 0.92-0.83 (m, 2H). ¹⁹F NMR (376 MHz, Methanol-d₄) δ −63.90.

### Example 125. 5-cyclopropyl-2-(4-cyclopropyl-6-methoxy-pyrimidin-5-yl)-7-[[4-[1-methyl-4-(trifluoromethyl)imidazol-2-yl]phenyl]methyl]pyrrolo[3,2-d]pyrimidine Compound 170

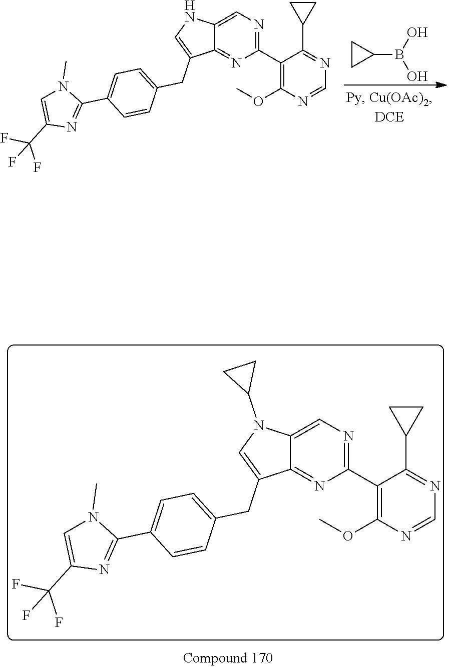

### 1.1 5-cyclopropyl-2-(4-cyclopropyl-6-methoxy-pyrimidin-5-yl)-7-[[4-[1-methyl-4-(trifluoromethyl)imidazol-2-yl]phenyl]methyl]pyrrolo[3,2-d]pyrimidine

To a solution of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (20 mg, 0.04 mmol) and cyclopropylboronic acid (5.2 mg, 60 μmol) in 1,2-dichloroethane (2 mL) were added pyridine (16 mg, 200 μmol, 16 μL) and cupric acetate (16 mg, 80 μmol) at room temperature. The mixture was stirred at 60° C. for 16 hours. The reaction mixture was cooled down to room temperature, 59% target product was detected by LCMS. Then the mixture was concentrated under reduced pressure. The residue was purified by RP-Flash with the following conditions: Column: C18 gel column 40 g, 20-35 μm; Mobile Phase A: Water (5 mM aq. NH₄HCO₃), Mobile Phase B: ACN: Gradient: 2% B hold 5 min, up to 61% B within 18 min, 61% B hold 5 min, up to 95% B within 5 min; Flow rate: 40 mL/min; Detector: UV 254 & 220 nm; RT: 26 min. The product-containing fractions were collected, concentrated and then lyophilized overnight to give 5-cyclopropyl-2-(4-cyclopropyl-6-methoxy-pyrimidin-5-yl)-7-[[4-[-methyl-4-(trifluoromethyl)imidazol-2-yl]phenyl]methyl]pyrrolo[3,2-d]pyrimidine (6.9 mg, 12.65 μmol, 31% yield) as a white solid. MS: m/z=546.20 [M+H]⁺. ¹H NMR (400 MHz, Chloroform-d) δ 9.31 (s, 1H), 8.79 (s, 1H), 7.63-7.52 (m, 3H), 7.45 (d, J=8.0 Hz, 2H), 7.34 (s, 1H), 4.27 (s, 2H), 4.10 (s, 3H), 3.79 (s, 3H), 3.56-3.53 (m, 1H), 1.94-1.91 (m, 1H), 1.49-1.42 (m, 2H), 1.31-1.23 (m, 2H), 1.19-1.12 (m, 2H), 1.06-0.98 (m, 2H). ¹⁹F NMR (376 MHz, Chloroform-d) δ −62.77.

### Example 126. 2-(2-(7-(4-(1-methyl-4-(trifluoromethyl)-1H-imidazol-2-yl)benzyl)-5H-pyrrolo[3,2-d]pyrimidin-2-yl)phenyl)propan-2-ol Compound 157

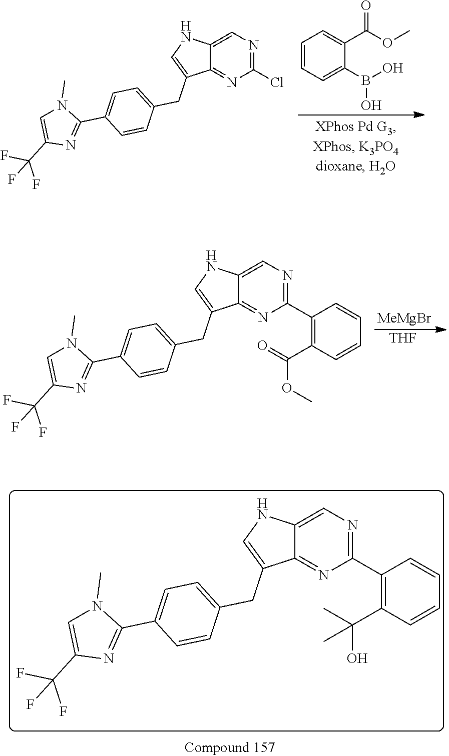

### 1.1 methyl 2-(7-(4-(1-methyl-4-(trifluoromethyl)-1H-imidazol-2-yl)benzyl)-5H-pyrrolo[3,2-d]pyrimidin-2-yl)benzoate

To the solution of 2-chloro-7-(4-(1-methyl-4-(trifluoromethyl)-1H-imidazol-2-yl)benzyl)-5H-pyrrolo[3,2-d]pyrimidine (200 mg, 510 μmol) and (2-methoxycarbonylphenyl)boronic acid (138 mg, 766 μmol) in water (0.8 mL) and 1,4-dioxane (4 mL) were added potassium phosphate (217 mg, 1.02 mmol), 2-Dicyclohexylphosphino-2′,4′,6′-triisopropylbiphenyl (24 mg, 51 μmol) and Methanesulfonato(2-dicyclohexylphosphino-2′,4′,6′-tri-i-propyl-1,1′-biphenyl)(2′-amino-1,1′-biphenyl-2-yl)palladium(II) (43 mg, 51 μmol) at 20° C. under nitrogen atmosphere. The reaction mixture was stirred at 90° C. for 16 hours. The mixture was cooled down to room temperature, 44% product was detected by LCMS. The resulted mixture was concentrated under reduced pressure. The residue was purified by Prep-TLC to give methyl 2-(7-(4-(1-methyl-4-(trifluoromethyl)-1H-imidazol-2-yl)benzyl)-5H-pyrrolo[3,2-d]pyrimidin-2-yl)benzoate (110 mg, 223.82 μmol, 43% yield) as a yellow solid. MS: m/z=492.10 [M+H]⁺. ¹H NMR (400 MHz, Chloroform-d) δ. 10.90 (s, 1H), 9.11 (s, 1H), 8.03 (d, J=7.6 Hz, 1H), 7.85 (d, J=7.6 Hz, 1H), 7.69-7.61 (m, 1H), 7.60-7.54 (m, 3H), 7.51 (s, 1H), 7.43 (d, J=7.6 Hz, 2H), 7.33 (s, 1H), 4.27 (s, 2H), 3.77 (s, 3H), 3.68 (s, 3H). ¹⁹F NMR (377 MHz, Chloroform-d) δ −62.61.

### 1.2 2-(2-(7-(4-(1-methyl-4-(trifluoromethyl)-1H-imidazol-2-yl)benzyl)-5H-pyrrolo[3,2-d]pyrimidin-2-yl)phenyl)propan-2-ol

To the solution of methyl 2-(7-(4-(1-methyl-4-(trifluoromethyl)-1H-imidazol-2-yl)benzyl)-5H-pyrrolo[3,2-d]pyrimidin-2-yl)benzoate (110 mg, 22.4 μmol) in tetrahydrofuran (4 mL) was added methylmagnesium bromide (2.24 mL, 2.24 mmol, 1 M in tetrahydrofuran) at 0° C. under nitrogen atmosphere. The reaction mixture was stirred at 20° C. for 16 hours, then was quenched with saturated ammonium chloride aqueous solution (20 mL). The resulted mixture was extracted with ethyl acetate (3×15 mL). The combined organic extracts were washed with brine (20 mL), dried over anhydrous sodium sulfate. After filtration, the filtrate was concentrated under reduced pressure. The residue was purified by Prep-TLC to give a crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN; Flow rate: 30 mL/min; Gradient: 5% B to 5% B in 5 min, 5% B to 50% B in 10 min. 50% B to 50% B in 2 min, 50% B to 95% B in 8 min; Detector: UV 254 & 220 nm; RT: 16 min. The collected fractions were combined, concentrated and then lyophilized overnight to give 2-(2-(7-(4-(1-methyl-4-(trifluoromethyl)-1H-imidazol-2-yl)benzyl)-5H-pyrrolo[3,2-d]pyrimidin-2-yl)phenyl)propan-2-ol (3 mg, 6.10 μmol, 2% yield) as a light-yellow solid. MS: m/z=492.25 [M +H]⁺. ¹H NMR (400 MHz, Chloroform-d) δ 10.54 (s, 1H), 8.80 (s, 1H), 7.90-7.84 (m, 1H), 7.65-7.59 (m, 1H), 7.54 (d, J=7.2 Hz, 2H), 7.48-4.38 (m, 4H), 7.33 (d, J=7.2 Hz, 2H), 4.25 (s, 2H), 3.77 (s, 3H), 1.45 (s, 6H). ¹⁹F NMR (377 MHz, Chloroform-d) δ −62.50.

### Example 127. 1-[2-(4-cyclopropyl-6-methoxy-pyrimidin-5-yl)-7-[[4-[1-methyl-4-(trifluoromethyl)imidazol-2-yl]phenyl]methyl]pyrrolo[3,2-d]pyrimidin-5-yl]ethenone Compound 93

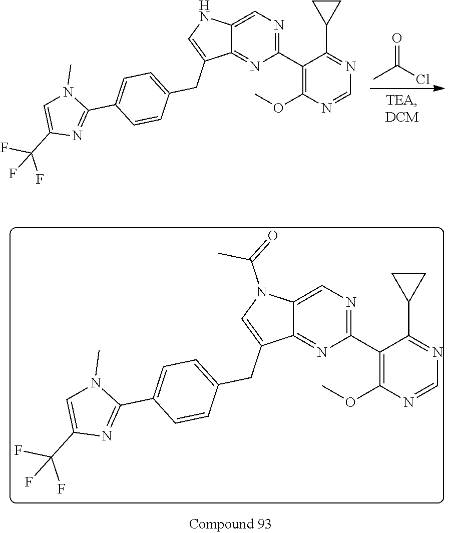

### 1.1 1-[2-(4-cyclopropyl-6-methoxy-pyrimidin-5-yl)-7-[[4-[1-methyl-4-(trifluoromethyl)imidazol-2-yl]phenyl]methyl]pyrrolo[3,2-d]pyrimidin-5-yl]ethanone

To a stirred mixture of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (100 mg, 198 μmol) in dichloromethane (1 mL) were added triethylamine (100.09 mg, 989.13 μmol, 137.87 μL) and acetyl chloride (62 mg, 791 μmol, 48 μL) under nitrogen atmosphere at 0° C. The resulting mixture was stirred at 40° C. for 16 hours. The reaction mixture was cooled down to room temperature. The reaction was monitored by LCMS. The reaction solution was concentrated under reduced pressure. The residue was purified by Prep-TLC, eluted with 3% methanol in dichloromethane to afford 50 mg crude product. The crude product was purified by reverse phase chromatography with the following conditions: C18 spherical Column, 20-35 um, 100A, 20 g; Mobile Phase A: Water (5 mM aq. NH₄HCO₃), Mobile Phase B: MeCN; Flow rate: 25 mL/min; Gradient: 5% B to 5% B in 5 min, 5% B to 60% B in 15 min, 60% B to 60% B in 3 min, 60% B to 95% B in 10 min; Detector: UV 254 & 210 nm: RT: 22 min. The product-containing fractions were collected and evaporated in vacuo and then lyophilized overnight to give 1-[2-(4-cyclopropyl-6-methoxy-pyrimidin-5-yl)-7-[[4-[1-methyl-4-(trifluoromethyl)imidazol-2-yl]phenyl]methyl]pyrrolo[3,2-d]pyrimidin-5-yl]ethanone (25.3 mg, 46.21 μmol, 23% yield) as an off-white solid. MS: m/z=548.30 [M+H]⁺. ¹H NMR (300 MHz, Chloroform-d) δ 9.80 (s, 1H), 8.67 (s, 1H), 7.61 (d, J=10.8 Hz, 2H), 7.48 (d, J=10.8 Hz, 2H), 7.38 (s, 1H), 7.33 (s, 1H), 4.24 (s, 2H), 3.93 (s, 3H), 3.78 (s, 3H), 2.62 (s, 3H), 1.72-1.60 (m, 1H), 1.29-1.18 (m, 2H), 0.93-0.80 (m, 2H). ¹⁹F NMR (282 MHz, Chloroform-d) δ −62.47.

### Example 128. 2-(4-cyclopropyl-6-methoxy-pyrimidin-5-yl)-5-methylsulfonyl-7-[[4-[1-methyl-4-(trifluoromethyl)imidazol-2-yl]phenyl]methyl]pyrrolo[3,2-d]pyrimidine Compound 146

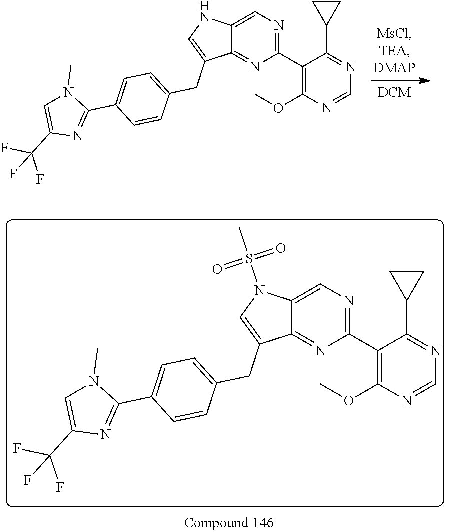 [SMILES: COc1ncnc(C2CC2)c1-c1ncc2[nH]cc(Cc3ccc(-c4nc(C(F)(F)F)cn4C)cc3)c2n1 ; COc1ncnc(C2CC2)c1-c1ncc2c(n1)c(Cc1ccc(-c3nc(C(F)(F)F)cn3C)cc1)cn2S(C)(=O)=O]

### 1.1 2-(4-cyclopropyl-6-methoxypyrimidin-5-yl)-7-(4-(1-methyl-4-(trifluoromethyl)-1H-imidazol-2-yl)benzyl)-5-(methylsulfonyl)-5H-pyrrolo[3,2-d]pyrimidine

To a stirred solution of 2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidine (50 mg, 99 μmol) in dichloromethane (2 mL) were added triethylamine (60 mg, 593 μmol, 83 μL), 4-dimethylaminopyridine (1.2 mg, 9.89 μmol) and methanesulfonyl chloride (57 mg, 495 μmol, 38 μL) at 0° C. under nitrogen atmosphere. The resulting mixture was stirred for 16 hours at 40° C. under nitrogen atmosphere. The reaction was monitored by LCMS. The mixture was concentrated under reduced pressure. The residue was purified by Prep-TLC, eluting with 10% methanol in methylene chloride to afford crude product. The obtained crude product was further purified by RP-Flash with the following conditions: C18 spherical Column, 20-30 um, 100A, 40 g; Mobile Phase A: 10 mM aq. NH₄HCO₃, Mobile Phase B: MeCN: Flow rate: 50 mL/min; Gradient: 5% B to 5% B in 5 min, 5% B to 61% B in 25 min; 61% B to 61% B in 5 min; 61% B to 95% B in 5 min; Detector: UV 254 & 220 nm; RT: 32 min. The collected fractions were combined, concentrated under reduced pressure and lyophilized to give 2-(4-cyclopropyl-6-methoxy-pyrimidin-5-yl)-5-methylsulfonyl-7-[[4-[1-methyl-4-(trifluoromethyl)imidazol-2-yl]phenyl]methyl]pyrrolo[3,2-d]pyrimidine (6 mg, 10.28 μmol, 10% yield) as a white solid. MS: m/z=584.30 [M+H]⁺. ¹H NMR (400 MHz, Chloroform-d) δ 9.42 (s, 1H), 8.72 (s, 1H), 7.63 (d, J=8.4 Hz, 2H), 7.50 (d, J=8.4 Hz, 2H), 7.48 (s, 1H), 7.34 (s, 1H), 4.26 (s, 2H), 3.97 (s, 3H), 3.80 (s, 3H), 3.30 (s, 3H), 1.70-1.66 (m, 11H), 1.32-1.26 (m, 2H), 0.95-0.89 (m, 2H). ¹⁹F NMR (376 MHz, Chloroform-d) δ −62.70.

### Example 129. 1-(5-(4-cyclopropyl-6-methoxypyrimidin-5-yl)-3-(4-(1-methyl-4-(trifluoromethyl)-1H-imidazol-2-yl)benzyl)-2H-pyrazolo[4,3-d]pyrimidin-2-yl)ethan-1-one Compound 101

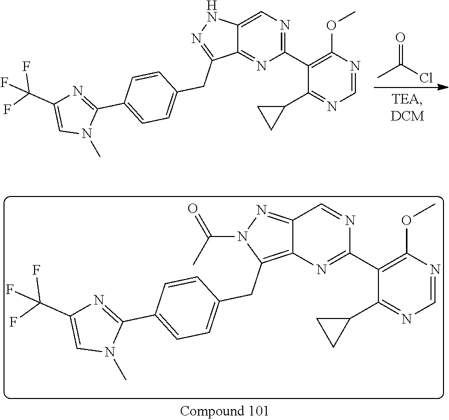

### 1.1 1-(5-(4-cyclopropyl-6-methoxypyrimidin-5-yl)-3-(4-(1-methyl-4-(trifluoromethyl)-1H-imidazol-2-yl)benzyl)-2H-pyrazolo[4,3-d]pyrimidin-2-yl)ethan-1-one

To a solution of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (100 mg, 197 μmol) and acetyl chloride (31 mg, 395 μmol, 24 μL) in dichloromethane (2 mL) was added triethylamine (80 mg, 790 μmol, 110 μL) at 25° C., the resulting reaction was stirred for 2 hours at 25° C. The resulting solution was detected by TLC and LCMS, could find 89% desired product. The residue was purified by Prep-TLC (PE:EA=1:2) to afford a crude product, then the crude product was further purified by RP-Flash chromatography with the following conditions: Column: C18 spherical, 20-30 um, 100A, 40 g; Mobile Phase A: 10 mM aq. NH₄HCO₃, Mobile Phase B: MeCN; Flow rate: 50 mL/min; Gradient: 5% B to 5% B in 5 min, 5% B to 72% B in 25 min; 72% B to 72% B in 5 min; 72% B to 95% B in 5 min; Detector: UV 254 & 220 nm; RT: 35 min. The collected fractions were combined, concentrated under reduced pressure and then lyophilized overnight to give 1-(5-(4-cyclopropyl-6-methoxypyrimidin-5-yl)-3-(4-(1-methyl-4-(trifluoromethyl)-RH-imidazol-2-yl)benzyl)-2H-pyrazolo[4,3-d]pyrimidin-2-yl)ethan-1-one (30 mg, 54.69 μmol, 28% yield) as an off-white solid. MS: m/z=549.20 [M+H]⁺. ¹H NMR (400 MHz, Chloroform-d) δ 9.40 (s, 1H), 8.69 (s, 1H), 7.61-7.55 (m, 4H), 7.30 (s, 1H), 4.52 (s, 2H), 3.92 (s, 3H), 3.74 (s, 3H), 2.82 (s, 3H), 1.66-1.60 (m, 1H), 1.26-1.21 (m, 2H), 0.89-0.83 (m, 2H). ¹⁹F NMR (376 MHz, Chloroform-d) δ −62.74.

### Example 130. 5-(4-cyclopropyl-6-methoxy-pyrimidin-5-yl)-2-methylsulfonyl-3-[[4-[1-methyl-4-(trifluoromethyl)imidazol-2-yl]phenyl]methyl]pyrazolo[4,3-d]pyrimidine Compound 164

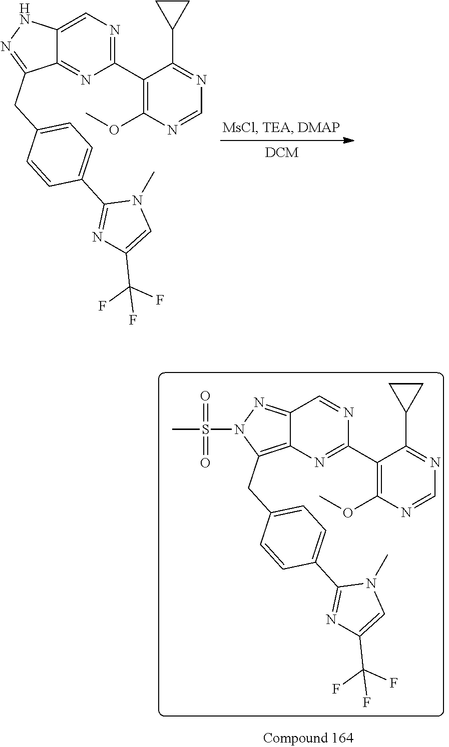 [SMILES: COc1ncnc(C2CC2)c1-c1ncc2[nH]nc(Cc3ccc(-c4nc(C(F)(F)F)cn4C)cc3)c2n1 ; COc1ncnc(C2CC2)c1-c1ncc2nn(S(C)(=O)=O)c(Cc3ccc(-c4nc(C(F)(F)F)cn4C)cc3)c2n1]

### 1.1 5-(4-cyclopropyl-6-methoxy-pyrimidin-5-yl)-2-methylsulfonyl-3-[[4-[1-methyl-4-(trifluoromethyl)imidazol-2-yl]phenyl]methyl]pyrazolo[4,3-d]pyrimidine

To a stirred solution of 5-(4-cyclopropyl-6-methoxy-pyrimidin-5-yl)-3-[[4-[1-methyl-4-(trifluoromethyl)imidazol-2-yl]phenyl]methyl]-1H-pyrazolo[4,3-d]pyrimidine (100 mg, 197 μmol) and methanesulfonyl chloride (34 mg, 296 μmol) in dichloromethane (2 mL) were added triethylamine (40 mg, 395 μmol) and N,N-dimethylpyridin-4-amine (cat.) at room temperature under nitrogen atmosphere. The mixture was stirred at room temperature for 1 hour under nitrogen atmosphere. The resulting solution was detected by LCMS, could find 20% desired product. The resulted reaction solution was concentrated under reduced pressure. The residue was purified by Prep-TLC (dichloromethane:methanol=15:1) to afford a crude product. The crude product was further purified by reverse phase chromatography with the following conditions: Column: Spherical C18, 20-40 um, 40 g; Mobile Phase A: Water (5 mM aq. NH₄HCO₃), Mobile Phase B: ACN: Flow rate: 40 ml/min; Gradient (B %): 5% hold 5 min, 5%-52% within 22 min, 52% hold 5 min; 52%-95% within 10 min. 95% hold 5 min; Detector: UV 254 & 210 nm; RT: 27 min. The collected fractions were combined, concentrated under reduced pressure and lyophilized to afford 5-(4-cyclopropyl-6-methoxy-pyrimidin-5-yl)-2-methylsulfonyl-3-[[4-[1-methyl-4-(trifluoromethyl)imidazol-2-yl]phenyl]methyl]pyrazolo[4,3-d]pyrimidine (14.5 mg, 24.80 μmol, 12% yield) as an off-white solid. MS: m/z=585.15 [M+H]⁺. ¹H NMR (400 MHz, Chloroform-d) δ 9.67 (s, 1H), 8.71 (s, 1H), 7.62-7.55 (m, 4H), 7.31 (s, 1H), 4.55 (s, 2H), 3.94 (s, 3H), 3.75 (s, 3H), 3.45 (s, 3H), 1.69-1.60 (m, 1H), 1.30-1.22 (m, 2H), 0.94-0.84 (m, 2H). ¹⁹F NMR (376 MHz, Chloroform-d) δ −62.76.

### Example 131. 1-[2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-6-yl]-N-methyl-methanamine Compound 159

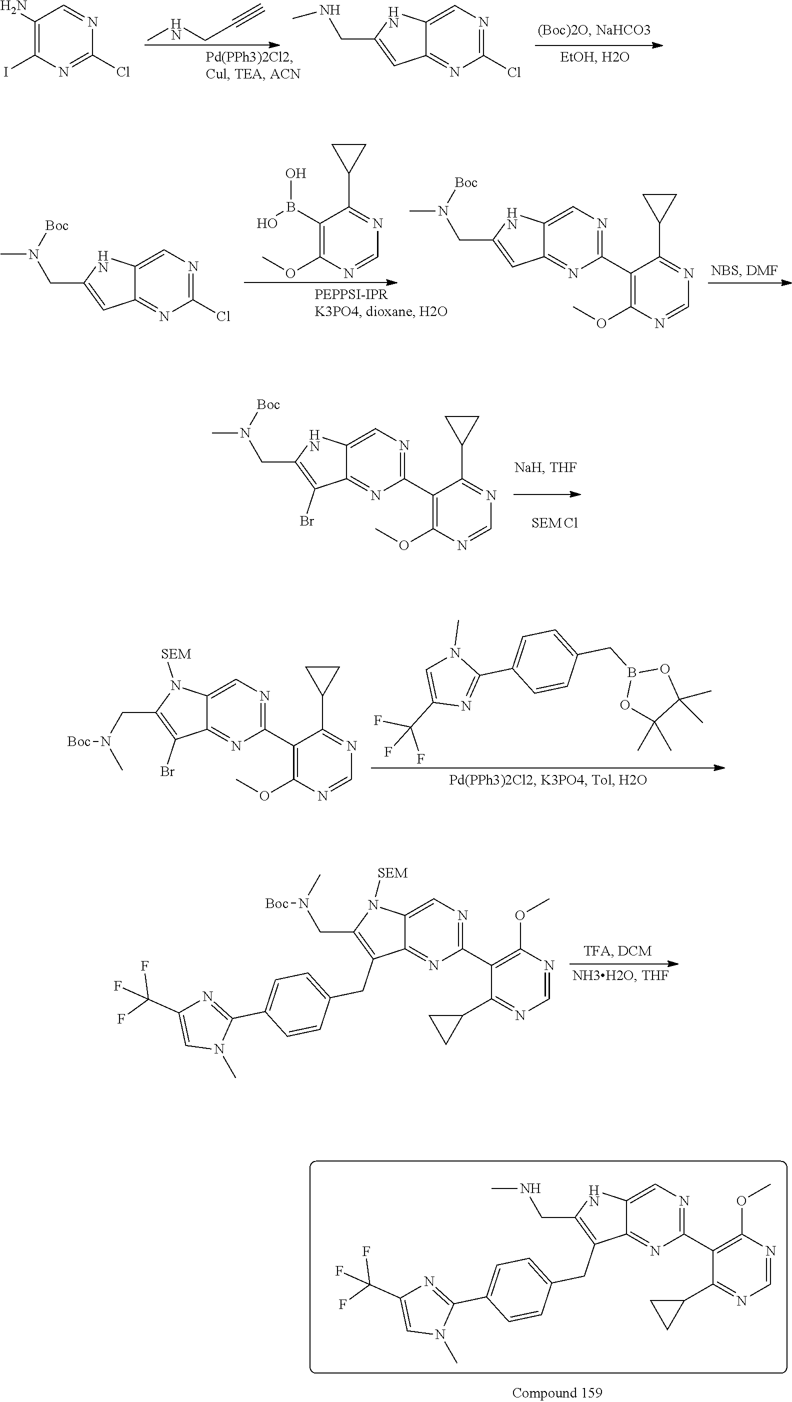

### 1.1 1-(2-chloro-5H-pyrrolo[3,2d]pyrimidin-6-yl)-N-methyl-methanamine

To a stirred solution of 2-chloro-4-iodo-pyrimidin-5-amine (800 mg, 3.13 mmol) and N-methylprop-2-yn-1-amine (281.35 mg, 4.07 mmol) in acetonitrile (10 mL) and triethylamine (10 mL) were added bis(triphenylphosphine)palladium dichloride (110 mg, 157 μmol) and iodocopper (36 mg, 188 μmol) at room temperature under nitrogen atmosphere. The mixture was stirred at room temperature for 0.5 hour under nitrogen atmosphere. The resulting mixture was concentrated under reduced pressure. The residue was purified by silica gel column chromatography, eluted with methanol in dichloromethane (0%-8%) to afford 1-(2-chloro-5H-pyrrolo[3,2-d]pyrimidin-6-yl)-N-methyl-methanamine (500 mg, 2.54 mmol, 81% yield) as a black oil. MS: m/z=197.10 [M+H]⁺. ¹H NMR (400 MHz, Methanol-d₄) δ 8.66 (s, 1H), 6.55 (s, 1H), 3.99 (s, 2H), 2.45 (s, 3H).

### 1.2 tert-butyl N-[(2-chloro-5H-pyrrolo[3,2-d]pyrimidin-6-yl)methyl]-N-methyl-carbamate

To a stirred mixture of 1-(2-chloro-5H-pyrrolo[3,2-d]pyrimidin-6-yl)-N-methyl-methanamine (500 mg, 2.54 mmol) in ethanol (10 mL) and water (10 mL) was added sodium bicarbonate (1.28 g, 15.26 mmol) and di-tert-butyl dicarbonate (3.33 g, 15.26 mmol) under nitrogen atmosphere at 25° C. The resulting mixture was stirred for 1 hour at 25° C. The reaction was quenched by the addition of ammonium chloride (100 mL). The mixture was extracted with ethyl acetate (3×100 mL). The combined organic layers were washed with brine (3×100 mL), dried over anhydrous sodium sulfate. After filtration, the filtrate was concentrated under reduced pressure. The residue was purified by silica gel column chromatography, eluted with 32%-33% ethyl acetate in petroleum ether to give tert-butyl N-[(2-chloro-5H-pyrrolo[3,2<d]pyrimidin-6-yl)methyl]-N-methyl-carbamate (600 mg, 2.02 mmol, 80% yield) as a yellow solid. MS: m/z=296.85 [M+H]⁺. ¹H NMR (400 MHz, Chloroform-d) δ 10.01 (s, 1H), 8.75 (s, 1H), 6.56 (s, 1H), 4.53 (s, 2H), 2.97 (s, 3H), 1.53 (s, 9H).

### 1.3 tert-butyl N-[[2-(4-cyclopropyl-6-methoxy-pyrimidin-5-yl)-5H-pyrrolo[3,2-d]pyrimidin-6-yl]methyl]-N-methyl-carbamate

To a stirred mixture of tert-butyl N-[(2-chloro-5H-pyrrolo[3,2-d]pyrimidin-6-yl)methyl]-N-methyl-carbamate (600 mg, 2.02 mmol), (4-cyclopropyl-6-methoxy-pyrimidin-5-yl)boronic acid (588 mg, 3.03 mmol) and potassium phosphate (858 mg, 4.04 mmol) in 1,4-dioxane (10 mL) and water (2 mL) was added [1,3-Bis(2,6-Diisopropylphenyl)imidazol-2-ylidene](3-chloropyridyl)palladium(II) dichloride (137 mg, 202 μmol) under nitrogen atmosphere at 25° C. The resulting mixture was stirred at 90° C. for 16 hours. The reaction was cooled down to room temperature, then diluted with water (200 mL), extracted with dichloromethane (3×10 mL). The combined organic layers were washed with brine (100 mL), dried over anhydrous sodium sulfate. After filtration, the filtrate was concentrated under reduced pressure. The residue was purified by silica gel column chromatography, eluted with 0%-60% ethyl acetate in petroleum ether to give tert-butyl N-[[2-(4-cyclopropyl-6-methoxy-pyrimidin-5-yl)-5H-pyrrolo[3,2-d]pyrimidin-6-yl]methyl]-N-methyl-carbamate (200 mg, 487.25 μmol, 24% yield) as a yellow solid. MS: m/z=411.20 [M+H]⁺. ¹H NMR (400 MHz, Chloroform-d) δ 10.06 (s, 1H), 9.06 (s, 1H), 8.68 (s, 1H), 6.68 (s, 1H), 4.58 (s, 2H), 3.94 (s, 3H), 2.99 (s, 3H), 2.15-2.11 (m, 1H), 1.54 (s, 9H), 1.31-1.25 (m, 2H), 0.93-0.89 (m, 2H).

### 1.4 tert-butyl N-[[7-bromo-2-(4-cyclopropyl-6-methoxy-pyrimidin-5-yl)-5H-pyrrolo[3,2-d]pyrimidin-6-yl]methyl]-N-methyl-carbamate

To a stirred mixture of tert-butyl N-[[2-(4-cyclopropyl-6-methoxy-pyrimidin-5-yl)-5H-pyrrolo[3,2-d]pyrimidin-6-yl]methyl]-N-methyl-carbamate (200 mg, 487 μmol) in N,N-dimethylformamide (3 mL) was added N-bromosuccinimide (87 mg, 487 μmol) under nitrogen atmosphere at 25° C. The resulting mixture was stirred for 2 hours at 25° C. The reaction was quenched by the addition of saturated ammonium chloride aqueous solution (100 mL). The resulted mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 45%-50% ethyl acetate in petroleum ether to give tert-butyl N-[[7-bromo-2-(4-cyclopropyl-6-methoxy-pyrimidin-5-yl)-5H-pyrrolo[3,2-d]pyrimidin-6-yl]methyl]—N-methyl-carbamate (200 mg, 408.69 μmol, 84% yield) as a yellow oil. MS: m/z=489.10, 491.10 [M+H]⁺. ¹H NMR (400 MHz, Chloroform-d) δ 9.03 (s, 1H), 8.67 (s, 1H), 4.65 (s, 2H), 3.92 (s, 3H), 3.03 (d, J=2.4 Hz, 3H), 2.17-2.05 (m, 1H), 1.54 (s, 9H), 1.28-1.23 (m, 2H), 0.91-0.88 (s, 2H).

### 1.5 tert-butyl N-[[7-bromo-2-(4-cyclopropyl-6-methoxy-pyrimidin-5-yl)-5-(2-trimethylsilylethoxymethyl)pyrrolo[3,2-d]pyrimidin-6-yl]methyl]-N-methyl-carbamate

To a stirred mixture of tert-butyl N-[[7-bromo-2-(4-cyclopropyl-6-methoxy-pyrimidin-5-yl)-5H-pyrrolo[3,2-d]pyrimidin-6-yl]methyl]-N-methyl-carbamate (200 mg, 408.69 μmol) in tetrahydrofuran (3 mL) was added sodium hydride (20 mg, 490 μmol, 60% purity) under nitrogen atmosphere at 0° C. The resulting mixture was stirred for 1 hour at 0° C. To the above mixture was added (2-(chloromethoxy)ethyl)trimethylsilane (82 mg, 490 μmol) under nitrogen atmosphere at 0° C. The resulted mixture was stirred at 25° C. for 2 hours. The reaction was quenched by the addition of ammonium chloride (100 mL). The resulting mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 32%-33% ethyl acetate in petroleum ether to give tert-butyl N-[[7-bromo-2-(4-cyclopropyl-6-methoxy-pyrimidin-5-yl)-5-(2-trimethylsilylethoxymethyl)pyrrolo[3,2-d]pyrimidin-6-yl]methyl]-N-methyl-carbamate (170 mg, 274.36 μmol, 67% yield) as a yellow oil. MS: m/z=621.20, 623.20 [M+H]⁺. ¹H NMR (400 MHz, Chloroform-d) δ 9.13 (s, 1H), 8.70 (s, 1H), 5.72 (s, 2H), 4.93 (s, 2H), 3.94 (s, 3H), 3.66-3.52 (m, 2H), 2.87 (s, 3H), 2.21-2.04 (m, 1H), 1.55 (s, 9H), 1.30 (s, 2H), 0.97-0.88 (m, 4H), −0.01 (s, 9H).

### 1.6 tert-butyl N-[[2-(4-cyclopropyl-6-methoxy-pyrimidin-5-yl)-7-[[4-[1-methyl-4-(trifluoromethyl)imidazol-2-yl]phenyl]methyl]-5-(2-trimethylsilylethoxymethyl)pyrrolo[3,2-d]pyrimidin-6-yl]methyl]-N-methyl-carbamate

To a stirred solution of tert-butyl N-[[7-bromo-2-(4-cyclopropyl-6-methoxy-pyrimidin-5-yl)-5-(2-trimethylsilylethoxymethyl)pyrrolo[3,2-d]pyrimidin-6-yl]methyl]-N-methyl-carbamate (170 mg, 274.36 μmol) and 1-methyl-2-[4-[(4,4,5,5-tetramethyl-1,3,2-dioxaborolan-2-yl)methyl]phenyl]-4-(trifluoromethyl)imidazole (301 mg, 823 μmol) in toluene (5 mL) and water (0.6 mL) were added bis(triphenylphosphine)palladium(II) chloride (58 mg, 82 μmol) and potassium phosphate (58 mg, 274 μmol) under nitrogen atmosphere at 25° C. The resulting mixture was stirred at 90° C. for 16 hours. The reaction was cooled down to room temperature, then quenched with water (100 mL), extracted with dichloromethane (3×50 mL). The combined organic layers were washed with brine (100 mL), dried over anhydrous sodium sulfate. After filtration, the filtrate was concentrated under reduced pressure. The residue was purified by silica gel column chromatography, eluted with 50%-60% ethyl acetate in petroleum ether to give tert-butyl N-[[2-(4-cyclopropyl-6-methoxy-pyrimidin-5-yl)-7-[[4-[-methyl-4-(trifluoromethyl)imidazol-2-yl]phenyl]methyl]-5-(2-trimethylsilylethoxymethyl)pyrrolo[3,2-d]pyrimidin-6-yl]methyl]-N-methyl-carbamate (100 mg, 128.38 μmol, 47% yield) as a yellow solid. MS: m/z=779.55 [M+H]⁺.

### 1.7 1-[2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-6-yl]-N-methyl-methanamine

To a stirred solution of tert-butyl N-[[2-(4-cyclopropyl-6-methoxy-pyrimidin-5-yl)-7-[[4-[-methyl-4-(trifluoromethyl)imidazol-2-yl]phenyl]methyl]-5-(2-trimethylsilylethoxymethyl)pyrrolo[3,2-d]pyrimidin-6-yl]methyl]-N-methyl-carbamate (50 mg, 64 μmol) in methylene chloride (1 mL) was added trifluoroacetic acid (1 mL) and then stirred for 2 hours at 25° C., then tetrahydrofuran (1 mL) and ammonium hydroxide (1 mL, 28% aqueous solution) were added in the above mixture, the resulted mixture was stirred for 30 min at 25° C. The resulted mixture was concentrated under reduced pressure. The residue was purified by Prep-TLC, eluted with 10% methanol in dichloromethane to give 40 mg crude product. The crude product was purified by RP-Flash with the following conditions: Column: C18 spherical Column, 20-35 um, 100A, 40 g; Mobile Phase A: Water (5 mM aq. NH₄HCO₃), Mobile Phase B: MeCN; Flow rate: 60 mL/min; Gradient: 5% B to 5% B in 5 min, 5% B to 50% B in 16 min. 50% B to 50% B in 4 min, 50% B to 95% B in 5 min; Detector: UV 254& 210 nm; RT: 24 min. The collected fractions were combined, concentrated and then lyophilized to afford 1-[2-(4-cyclopropyl-6-methoxy-pyrimidin-5-yl)-7-[[4-[1-methyl-4-(trifluoromethyl)imidazol-2-yl]phenyl]methyl]-5H-pyrrolo[3,2-d]pyrimidin-6-yl]-N-methyl-methanamine (27.0 mg, 49.22 μmol, 77% yield) as an off-white solid. MS: m/z=549.40 [M+H]⁺. ¹H NMR (400 MHz, Chloroform-d) δ 8.97 (s, 1H), 8.67 (s, 1H), 7.46 (d, J=7.6 Hz, 2H), 7.38 (d, J=7.6 Hz, 2H), 7.32 (s, 1H), 4.28 (s, 2H), 4.00 (s, 2H), 3.94 (s, 3H), 3.73 (s, 3H), 2.43 (s, 3H), 1.78-1.71 (m, 1H), 1.24-1.21 (m, 2H), 0.87-0.83 (m, 2H). ¹⁹F NMR (377 MHz, Chloroform-d) δ −62.61.

### Example 132. [2-(4-cyclopropyl-6-methoxy-pyrimidin-5-yl)-6-(2-methoxyethyl)-5H-pyrrolo[3,2-d]pyrimidin-7-yl]-[4-[1-methyl-4-(trifluoromethyl)imidazol-2-yl]phenyl]methanol Compound 109

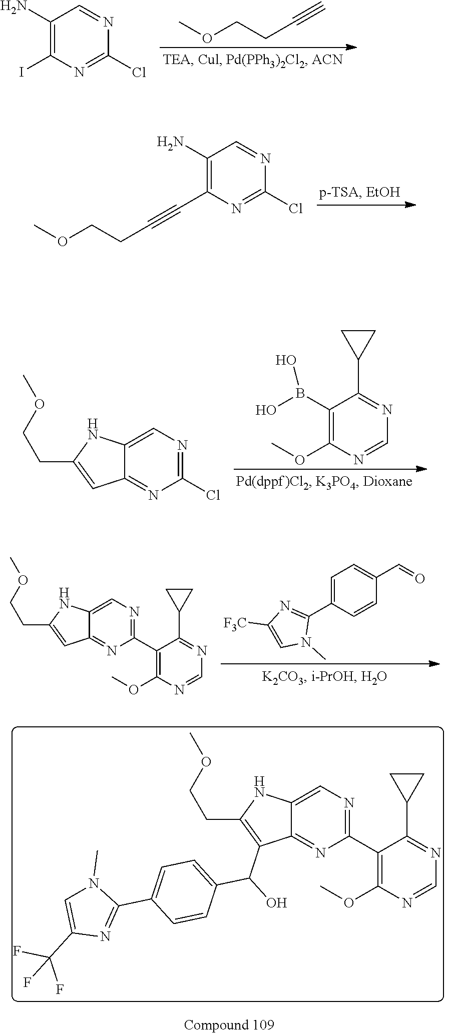

### 1.1 2-chloro-4-(4-methoxybut-1-ynyl)pyrimidin-5-amine

To a solution of 2-chloro-4-iodo-pyrimidin-5-amine (1.1 g, 4.31 mmol) in acetonitrile (6 mL) was added triethylamine (6 mL). Degas the solution. To the above mixture were added 4-methoxybut-1-yne (471 mg, 5.60 mmol), iodocopper (49 mg, 258 μmol) and dichloropalladium triphenylphosphane (151 mg, 215 μmol). The resulting mixture was stirred at room temperature for 30 min. The reaction was concentrated under reduced pressure. The residue was purified by silica gel column chromatography, eluted with methanol in dichloromethane (0%-3%) to afford 2-chloro-4-(4-methoxybut-1-ynyl)pyrimidin-5-amine (0.9 g, 4.25 mmol, 98% yield) as a dark yellow solid. MS: m/z=212.00, 213.95 [M+H]⁺. ¹H NMR (400 MHz, Chloroform-d) δ 8.13 (s, 1H), 4.38 (s, 2H), 3.63 (t, J=6.4 Hz, 2H), 3.41 (s, 3H), 2.81 (t, J=6.4 Hz, 2H).

### 1.2 2-chloro-6-(2-methoxyethyl)-5H-pyrrolo[3,2-d]pyrimidine

To a stirred solution of 2-chloro-4-(4-methoxybut-1-ynyl)pyrimidin-5-amine (900 mg, 4.25 mmol) in ethanol (10 mL) was added 4-methylbenzenesulfonic acid hydrate (809 mg, 4.25 mmol). The mixture was stirred at 80° C. for 16 hours. The resulted mixture was diluted with water (100 mL). The resulting mixture was extracted with dichloromethane (3×100 mL). The combined organic layers were dried over anhydrous sodium sulfate. After filtration, the filtrate was concentrated under reduced pressure and the residue was purified by silica gel column chromatography, eluted with methanol in dichloromethane (0%-9%) to afford 2-chloro-6-(2-methoxyethyl)-5H-pyrrolo[3,2-d]pyrimidine (0.37 g, 1.75 mmol, 41% yield) as a yellow solid. MS: m/z=211.95, 213.95 [M+H]+. ¹H NMR (400 MHz, Chloroform-d) δ 9.49 (s, 1H), 8.63 (s, 1H), 6.43-6.38 (m, 1H), 3.77 (t, J=5.6 Hz, 2H), 3.49 (s, 3H), 3.14 (t, J=5.6 Hz, 2H).

### 1.3 2-(4-cyclopropyl-6-methoxy-pyrimidin-5-yl)-6-(2-methoxyethyl)-5H-pyrrolo[3,2-d]pyrimidine

To a solution of 2-chloro-6-(2-methoxyethyl)-5H-pyrrolo[3,2-d]pyrimidine (0.37 g, 1.75 mmol) and (4-cyclopropyl-6-methoxy-pyrimidin-5-yl)boronic acid (407 mg, 2.10 mmol) in 1,4-dioxane (5 mL) and water (1 mL) were added [1,1′-Bis(diphenylphosphino)ferrocene]dichloropalladium(II) complex with dichloromethane (143 mg, 175 μmol) and potassium phosphate (1.11 g, 5.24 mmol). The mixture solution was stirred for 16 hours at 100° C. under nitrogen atmosphere. The resulting mixture was concentrated under reduced pressure. The residue was purified by silica gel column chromatography, eluted with (dichloromethane:methanol=9:1) to afford 2-(4-cyclopropyl-6-methoxy-pyrimidin-5-yl)-6-(2-methoxyethyl)-5H-pyrrolo[3,2-d]pyrimidine (0.2 g, 614.70 μmol, 35% yield) as a dark yellow oil, 2-chloro-6-(methoxymethyl)-5H-pyrrolo[3,2-d]pyrimidine (0.14 g) was recycled. MS: m/z=326.05 [M+H]⁺. ¹H NMR (400 MHz, Chloroform-d) δ 9.59 (s, 1H), 8.94 (s, 1H), 8.66 (s, 1H), 6.54 (s, 1H), 3.91 (s, 3H), 3.82-3.72 (m, 2H), 3.50 (s, 3H), 3.21-3.06 (m, 2H), 1.76-1.64 (m, 1H), 1.25-1.18 (m, 2H), 0.93-0.84 (m, 2H).

### 1.4 [2-(4-cyclopropyl-6-methoxy-pyrimidin-5-yl)-6-(2-methoxyethyl)-5H-pyrrolo[3,2-d]pyrimidin-7-yl]-[4-[1-methyl-4-(trifluoromethyl)imidazol-2-yl]phenyl]methanol

To a stirred solution of 2-(4-cyclopropyl-6-methoxy-pyrimidin-5-yl)-6-(2-methoxyethyl)-5H-pyrrolo[3,2-d]pyrimidine (200 mg, 614.70 μmol) in isopropyl alcohol (3 mL) and water (3 mL) were added potassium carbonate (102 mg, 738 μmol) and 4-[1-methyl-4-(trifluoromethyl)imidazol-2-yl]benzaldehyde (156 mg, 615 μmol) at 0° C. The mixture was stirred at 60° C. for 16 hours. The resulted mixture was diluted with water (100 mL). The mixture solution was extracted with methanol in dichloromethane (10%, 400 mL). The combined organic layers were dried over anhydrous sodium sulfate. After filtration, the filtrate was concentrated under reduced pressure and the residue was purified by Prep-TLC (dichloromethane:methanol =10:1) to afford a mixture of desired product and by-product. The mixture was further purified by reverse phase chromatography with the following conditions: Column: Spherical C18, 20-40 um, 40 g; Mobile Phase A: Water (5 mM aq. NH₄HCO₃). Mobile Phase B: ACN; Flow rate: 40 mL/min; Gradient (B %): 5% hold 5 min, 5%-43% within 30 min, 43% hold 2 min; 43%-95% within 10 min, 95% hold 5 min; Detector: UV 254 & 210 nm; RT: 35 min. The collected fractions were combined and concentrated under reduced pressure to afford [2-(4-cyclopropyl-6-methoxy-pyrimidin-5-yl)-6-(2-methoxyethyl)-5H-pyrrolo[3,2-d]pyrimidin-7-yl]-[4-[1-methyl-4-(trifluoromethyl)imidazol-2-yl]phenyl]methanol (40 mg, 69.02 μmol, 11% yield) as an off-white solid and 115 mg recycled. MS: m/z=580.25 [M+H]⁺. ¹H NMR (400 MHz, Chloroform-d) δ 9.92 (s, 1H), 8.97 (s, 1H), 8.67 (s, 1H), 7.64-7.53 (m, 4H), 7.34 (s, 1H), 6.35 (s, 1H), 4.80 (s, 1H), 3.95 (s, 3H), 3.76 (s, 3H), 3.67-3.56 (m, 2H), 3.42 (s, 3H), 3.04-2.85 (m, 2H), 1.86-1.75 (m, 1H), 1.29-1.18 (m, 2H), 0.94-0.82 (m, 2H). ¹⁹F NMR (377 MHz, Chloroform-d) δ −62.67.

### Example 133. 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine Compound 99

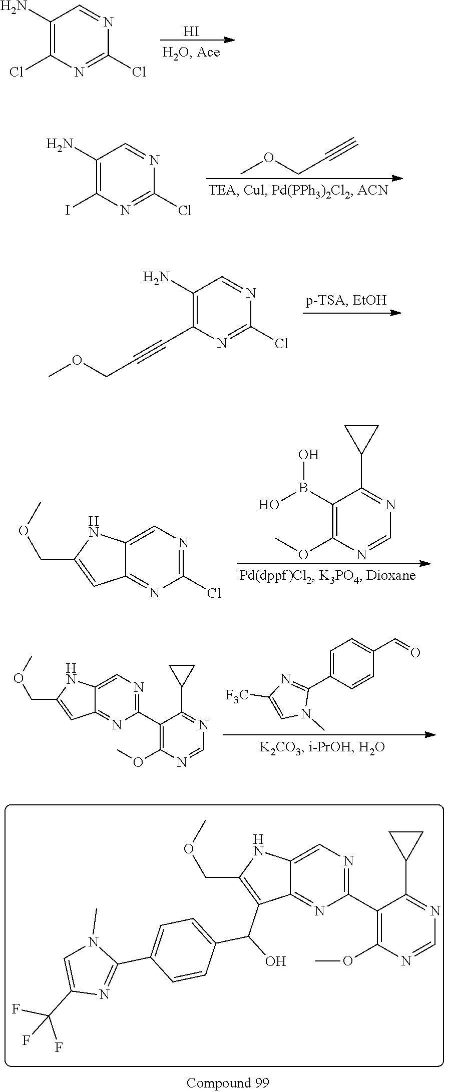

### 1.1. 2-chloro-4-iodo-pyrimidin-5-amine

To a stirred solution of 2,4-dichloropyrimidin-5-amine (2 g, 12.20 mmol) in acetone (10 mL) and water (10 mL) was added hydrogen iodide (57% aqueous solution, 30 mL) dropwise at room temperature. The mixture was stirred for 6 hours at room temperature. The resulted mixture was quenched with saturated sodium bicarbonate aqueous solution (350 mL). The resulted mixture was extracted with ethyl acetate (3×200 mL). The combined organic layers were dried over anhydrous sodium sulfate. After filtration, the filtrate was concentrated under reduced pressure and the residue was purified by silica gel column chromatography, eluted with ethyl acetate in petroleum ether (0%-47%) afford 2-chloro-4-iodo-pyrimidin-5-amine (crude) as a yellow solid. The crude product was further purified by reverse phase chromatography with the following conditions: Column: Spherical C18, 20-40 um, 40 g; Mobile Phase A: Water (5 mM aq. NH₄HCO₃), Mobile Phase B: ACN; Flow rate: 40 mL/min; Gradient (B %): 5% hold 5 min, 5%-31% within 16 min, 31% hold 2 min; 31%-95% within 10 min, 95% hold 5 min; Detector: UV 254 & 210 nm; RT: 21 min. The collected fractions were combined and concentrated under reduced pressure to afford 2-chloro-4-iodo-pyrimidin-5-amine (2.3 g, 9.00 mmol, 73% yield) as a light yellow solid. MS: m/z=255.95 [M+H]⁺. ¹H NMR (400 MHz, DMSO-d₆) δ 7.90 (s, 1H), 5.85 (s, 2H).

### 1.2 2-chloro-4-(3-methoxyprop-1-ynyl)pyrimidin-5-amine

To a solution of 2-chloro-4-iodo-pyrimidin-5-amine (2.2 g, 8.62 mmol) in acetonitrile (12 mL) and triethylamine (12 mL) were added 3-methoxyprop-1-yne (785 mg, 11 mmol), iodocopper (98 mg, 517 μmol) and bis(triphenylphosphine)palladium(II) chloride (302 mg, 431 μmol). The resulting mixture was stirred at room temperature for 30 min. The reaction mixture was filtered. The filtrate was concentrated under reduced pressure. The residue was purified by silica gel column chromatography, eluted with ethyl acetate in petroleum ether (0%-50%) to afford 2-chloro-4-(3-methoxyprop-1-ynyl)pyrimidin-5-amine (1.6 g, 8.10 mmol, 94% yield) as a yellow solid. MS: m/z=197.95 [M+H]⁺. ¹H NMR (400 MHz, Methanol-d₄) δ 8.19 (s, 1H), 4.46 (s, 2H), 3.48 (s, 3H).

### 1.3 2-chloro-6-(methoxymethyl)-5H-pyrrolo[3,2-d]pyrimidine

To a stirred solution of 2-chloro-4-(3-methoxyprop-1-ynyl)pyrimidin-5-amine (1.37 g, 6.93 mmol) in alcohol (15 mL) was added 4-methylbenzenesulfonic acid hydrate (1.32 g, 6.93 mmol) at room temperature. The mixture was stirred at 80° C. for 16 hours. The resulted mixture was cooled down to room temperature and the solid was filtered out. The filtrate was concentrated under reduced pressure. The residue was purified by silica gel column chromatography, eluted with methanol in dichloromethane (0%-7%) to afford crude product as a yellow solid. The crude product was further purified by reverse phase chromatography with the following conditions: Column: Spherical C18, 20-40 um, 80 g; Mobile Phase A: Water (5 mM NH₄HCO₃), Mobile Phase B: ACN; Flow rate: 60 mL/min; Gradient (B %): 5% hold 5 min, 5%-16% within 10 min; 16% hold 3 min; 16%-95% within 10 min, 95% hold 5 min; Detector: UV 254 & 210 nm, RT: 16 min. The collected fractions were combined and concentrated under reduced pressure to afford 2-chloro-6-(methoxymethyl)-5H-pyrrolo[3,2-d]pyrimidine (0.6 g, 3.04 mmol, 43% yield) as an off-white solid. MS: m/z=198.10 [M+H]⁺. ¹H NMR (400 MHz. Methanol-d₄) δ 8.67 (s, 1H), 6.52 (s, 1H), 4.73 (s, 2H), 3.47 (s, 3H).

### 1.4 2-(4-cyclopropyl-6-methoxy-pyrimidin-5-yl)-6-(methoxymethyl)-5H-pyrrolo[3,2-d]pyrimidine

To a solution of 2-chloro-6-(methoxymethyl)-5H-pyrrolo[3,2-d]pyrimidine (0.62 g, 3.14 mmol) and (4-cyclopropyl-6-methoxy-pyrimidin-5-yl)boronic acid (730. mg, 3.76 mmol) in 1,4-dioxane (6 mL) were added [11′-Bis(diphenylphosphino)ferrocene]dichloropalladium(1) complex with dichloromethane (257 mg, 313 μmol) and potassium phosphate (2.00 g, 9.41 mmol). The solution was stirred at 100° C. for 4 hours under nitrogen atmosphere. The resulted mixture was diluted with water (100 mL). The resulting mixture was extracted with ethyl acetate (3×100 mL). The combined organic layers were dried over anhydrous sodium sulfate. After filtration, the filtrate was concentrated under reduced pressure. The residue was purified by silica gel column chromatography, eluted with methanol in dichloromethane (0%-6%) to afford 2-(4-cyclopropyl-6-methoxy-pyrimidin-5-yl)-6-(methoxymethyl)-5H-pyrrolo[3,2-d]pyrimidine (0.6 g, 1.93 mmol, 61% yield) as a yellow solid and 140 mg 2-chloro-6-(methoxymethyl)-5H-pyrrolo[3,2-d]pyrimidine (0.14 g) was recycled. MS: m/z=312.05 [M+H]⁺. ¹H NMR (400 MHz, Chloroform-d) δ 9.70 (s, 1H), 8.91 (s, 1H), 8.64 (s, 1H), 6.65 (s, 1H), 4.72 (s, 2H), 3.89 (s, 3H), 3.48 (s, 3H), 1.73-1.62 (m, 1H), 1.25-1.17 (m, 2H), 0.92-0.83 (m, 2H).

### 1.5 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine

To a stirred solution of 2-(4-cyclopropyl-6-methoxy-pyrimidin-5-yl)-6-(methoxymethyl)-5H-pyrrolo[3,2-d]pyrimidine (0.5 g, 1.61 mmol) in water (2.5 mL) and isopropyl alcohol (2.5 mL) was added potassium carbonate (266 mg, 1.93 mmol) and 4-[1-methyl-4-(trifluoromethyl)imidazol-2-yl]benzaldehyde (408 mg, 1.61 mmol) at 0° C. The mixture was stirred at 60° C. for 6 h, 30% desired product could be detected on LCMS, major was starting materials. The resulting mixture was diluted with ethyl acetate (100 mL). The mixture reaction was extracted with ethyl acetate (3×100 mL). The combined organic layers were dried over anhydrous sodium sulfate. After filtration, the filtrate was concentrated under reduced pressure. The residue was purified by silica gel column chromatography, eluted with methanol in dichloromethane (0%-7%) to afford a crude product (220 mg) and 375 mg 2-(4-cyclopropyl-6-methoxy-pyrimidin-5-yl)-6-(methoxymethyl)-5H-pyrrolo[3,2-d]pyrimidine was recycled.

The crude product (10 mg) was purified by reverse phase chromatography with the following conditions: Column: Spherical C18, 20-40 um, 40 g; Mobile Phase A: Water (5 mM aq. NH₄HCO₃), Mobile Phase B: ACN; Flow rate: 40 mL/min; Gradient (B %): 5% hold 5 min, 5%-42% within 22 min, 42% hold 2 min; 42%-95% within 10 min, 95% hold 5 min; Detector: UV 254 & 210 nm; RT: 28 min. The collected fractions were combined and concentrated under reduced pressure to afford 2-(4-cyclopropyl-6-methoxy-pyrimidin-5-yl)-6-(methoxymethyl)-7-[[4-[1-methyl-4-(trifluoromethyl)imidazol-2-yl]phenyl]methyl]-5H-pyrrolo[3,2-d]pyrimidine (5.6 mg) as an off-white solid. MS: m/z=566.30 [M+H]⁺. ¹H NMR (400 MHz, Chloroform-d) δ 9.97 (s, 1H), 9.04 (s, 1H), 8.69 (s, 1H), 7.60 (s, 4H), 7.34 (s, 1H), 6.40 (s, 1H), 4.58-4.43 (m, 2H), 3.96 (s, 3H), 3.77 (s, 3H), 3.34 (s, 3H), 1.84-1.74 (m, 1H), 1.25-1.23 (m, 2H), 0.94-0.86 (m, 2H). ¹⁹F NMR (376 MHz, Chloroform-d) δ −62.66.

### Example 134. [4-[1-methyl-4-(trifluoromethyl)imidazol-2-yl]phenyl]-[2-[2-methyl-4-(trifluoromethyl)pyrazol-3-yl]-5H-pyrrolo[3,2-d]pyrimidin-7-yl]methanol Compound 108

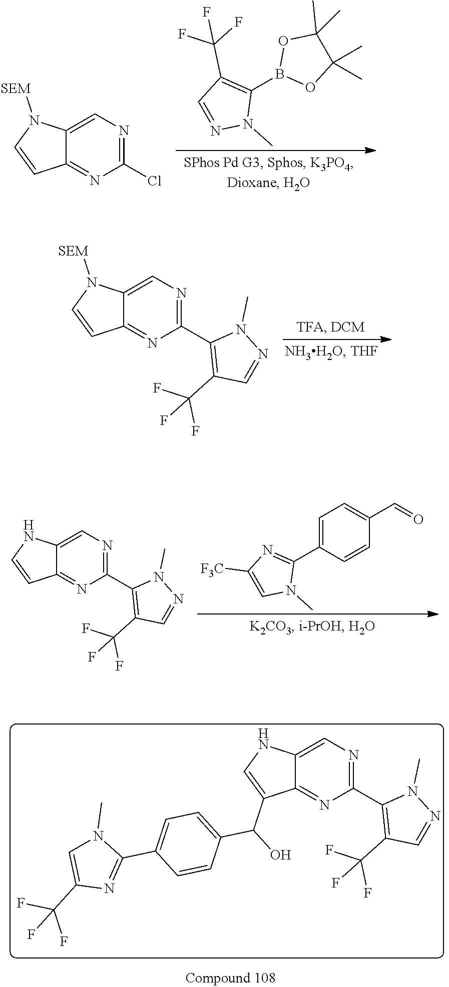 [SMILES: C[Si](C)(C)CCOCn1ccc2nc(Cl)ncc21 ; Cn1ncc(C(F)(F)F)c1B1OC(C)(C)C(C)(C)O1 ; Cn1ncc(C(F)(F)F)c1-c1ncc2c(ccn2COCC[Si](C)(C)C)n1 ; Cn1ncc(C(F)(F)F)c1-c1ncc2[nH]ccc2n1 ; Cn1cc(C(F)(F)F)nc1-c1ccc(C=O)cc1 ; Cn1cc(C(F)(F)F)nc1-c1ccc(C(O)c2c[nH]c3cnc(-c4c(C(F)(F)F)cnn4C)nc23)cc1]

### 1.1 trimethyl-[2-[[2-[2-methyl-4-(trifluoromethyl)pyrazol-3-yl]pyrrolo[3,2-d]pyrimidin-5-yl]methoxy]ethyl]silane

To a solution of 2-[(2-chloropyrrolo[3,2-d]pyrimidin-5-yl)methoxy]ethyl-trimethyl-silane (500 mg, 1.76 mmol) and 1-methyl-5-(4,4,5,5-tetramethyl-1,3,2-dioxaborolan-2-yl)-4-(trifluoromethyl)pyrazole (632.21 mg, 2.29 mmol) in dioxane (12.5 mL) and water (2.5 mL) under nitrogen atmosphere were added methanesulfonato(2-Dicyclohexylphosphino-2,6-dimethoxybiphenyl)(2-amino-1,1-biphenyl-2-yl)palladium(II) (153 mg, 176 μmol), potassium phosphate (748 mg, 3.52 mmol) and 2-Dicyclohexylphosphino-2′,6′-dimethoxybiphenyl (72 mg, 176 μmol) at 25° C. The reaction mixture was stirred at 100° C. for 16 hours. The mixture was cooled down to 25° C., diluted with 50 ml of water, then extracted with ethyl acetate (3×20 mL). The combined organic layers were dried over anhydrous sodium sulfate and filtered. The filtrate was concentrated under reduced pressure. The residue was purified by Prep-TLC, eluted with 50% ethyl acetate in petroleum ether to give trimethyl-[2-[[2-[2-methyl-4-(trifluoromethyl)pyrazol-3-yl]pyrrolo[3,2-d]pyrimidin-5-yl]methoxy]ethyl]silane (353 mg, 888.12 μmol, 50% yield) as a light-yellow solid. MS: m/z=398.15 [M+H]⁺. ¹H NMR (400 MHz, Chloroform-d) δ 9.13 (s, 1H), 7.80 (s, 1H), 7.68 (d, J=3.6 Hz, 1H), 6.83 (d, J=3.2 Hz, 1H), 5.61 (s, 2H), 4.14 (s, 3H), 3.59-3.55 (m, 2H), 1.00-0.94 (m, 2H), −0.02 (s, 9H). ¹⁹F NMR (376 MHz, Chloroform-d) δ −55.75.

### 1.2 2-[2-methyl-4-(trifluoromethyl)pyrazol-3-yl]-5H-pyrrolo[3,2-d]pyrimidine

To a solution of trimethyl-[2-[[2-[2-methyl-4-(trifluoromethyl)pyrazol-3-yl]pyrrolo[3,2-d]pyrimidin-5-yl]methoxy]ethyl]silane (350 mg, 880 μmol) in dichloromethane (4 mL) was added 2,2,2-trifluoroacetic acid (5.92 g, 51.92 mmol, 4 mL) at 0° C. under nitrogen atmosphere. The reaction mixture was stirred at 25° C. for 2 hours. The reaction solution was concentrated under reduced pressure. The residue was dissolved in tetrahydrofuran (4 mL) and then ammonium hydroxide (3.60 g, 102.72 mmol, 4 mL) was added at 0° C. After stirred at 25° C. for 1 hour, the resulted mixture was concentrated under reduced pressure. The residue was purified by silica gel column chromatography, eluted with 2%-10% methanol in dichloromethane to give 2-[2-methyl-4-(trifluoromethyl)pyrazol-3-yl]-5H-pyrrolo[3,2-d]pyrimidine (200 mg, 748.48 μmol, 85% yield) as a light-yellow solid. MS: m/z=268.05 [M+H]⁺.

### 1.3 [4-[1-methyl-4-(trifluoromethyl)imidazol-2-yl]phenyl]-[2-[2-methyl-4-(trifluoromethyl)pyrazol-3-yl]-5H-pyrrolo[3,2-d]pyrimidin-7-yl]methanol

To a solution of 2-[2-methyl-4-(trifluoromethyl)pyrazol-3-yl]-5H-pyrrolo[3,2-d]pyrimidine (10 mg, 37 μmol) and 4-[1-methyl-4-(trifluoromethyl)imidazol-2-yl]benzaldehyde (10 mg, 39 μmol) in isopropyl alcohol (0.1 mL) under nitrogen atmosphere were added potassium carbonate (6.2 mg, 45 μmol) and water (0.1 mL) at 25° C. The reaction mixture was stirred at 60° C. for 16 hours. The resulted mixture was cooled down to 25° C., diluted with water (5 mL), then extracted with ethyl acetate (3×10 ml). The combined organic layers were washed with brine (5 mL), dried over anhydrous sodium sulfate and filtered. The filtrate was concentrated under reduced pressure. The residue was purified by Prep-TLC, eluted with 10% methanol in dichloromethane to give a crude product. The crude product was purified by RP-Flash with the following conditions: Column: C18 spherical Column, 20-35 um, 100A, 20 g; Mobile Phase A: Water (5 mM aq. NH₄HCO₃), Mobile Phase B: MeCN: Flow rate: 5% B to 5% B in 3 min, 5% B to 46% B in 15 min, 46% B to 46% B in 3 min, 46% B to 95% B in 10 min, 95% B to 95% B in 2 min; Detector: UV 254 & 210 nm; RT: 19 min. The collected fractions were combined, concentrated and then lyophilized overnight to give [4-[1-methyl-4-(trifluoromethyl)imidazol-2-yl]phenyl]-[2-[2-methyl-4-(trifluoromethyl)pyrazol-3-yl]-5H-pyrrolo[3,2-d]pyrimidin-7-yl]methanol (6.1 mg, 11.70 μmol, 31% yield) as an off-white solid. MS: m/z=522.15 [M+H]⁺. ¹H NMR (400 MHz, Chloroform-d) δ 10.84 (s, 1H), 8.90 (s, 1H), 7.79 (s, 1H), 7.63-7.54 (m, 4H), 7.40 (s, 1H), 7.07 (s, 1H), 6.29 (s, 1H), 4.46 (s, 1H), 4.17 (s, 3H), 3.79 (s, 3H). ¹⁹F NMR (376 MHz, Chloroform-d) δ −55.74, −62.32.

### Example 135. Measurement of Inhibition of Deubiquitinase Activity by Exemplary Compounds

Deubiquitinase activity of USP1-UAF1 was measured using Ubiquitin-rhodamine 110 as a substrate. Cleavage of amide bond between rhodamine and C-terminal Glycine of Ubiquitin peptide yields Rhodamine 110-Gly, leading to an increase of fluorescence signal. The assay buffer consisted of 50 mM HEPES (pH 7.0), 1% DMSO, 0.01% Bovine Serum Albumin, 1 mM TCEP, 0.005% Tween-20. Total assay volume was 20 μL. Compounds depicted below were dissolved in 10 mM DMSO stock and enzyme inhibition was measured in dose response format with top concentration of 10 μM in final assay well, 10 μL of enzyme buffer mix consisting of 1 nM USP1-UAF1 in the assay buffer describe above was added to compounds and incubated at ambient temperature for 30 min. 10 μL of substrate mix consisting of 200 nM Ubiquitin-Rho110 was added to initiate the deubiquitinate reaction catalyzed by USP1/UAF1. End point fluorescence intensity of USP1/UAF1 deubiquitinase product, Rhodamine 110-Gly, was measured at Excitiation of 480 nm/Emission at 540 nm.

Percentage of activity was calculated by normalization of fluorescence intensity to control wells using the following equations: % Activity=100*((FI_{observed}−Min)/(Max−Min)−1) where FI_{observed} is the fluorescence intensity read out of the compound of interest samples, Min and Max is the fluorescence intensity of control well samples consisting of 1 mM of known USP1-UAF1 inhibitor probe ML-323 and DMSO controls respectively. IC₅₀ values were calculated using the standard dose response fit in Genedata Screener® where the top and bottom were fixed to 0 and −100 respectively.

### Example 136. Cellular Viability Assay

For short-term viability assays, cells were seeded in triplicates in 384-well plates one day prior to compound addition. Cells were incubated for 10 days with DMSO and increasing concentration of compounds. Cell viability was determined at end of the assay using Cell Titer-Glo Luminescence Assay (Promega) following manufacturer's instructions using an EnVision plate reader (Perkin Elmer). Measured values were normalized using DMSO control wells (100%) and complete cell kill control (0%; 10 μM MG132).

For long-term colony formation assays, cells were seeded in 12-well or 6-well plates at a very low density one day prior to compound addition. Cells were incubated for 7-21 days, depending on the cell line doubling time, with DMSO and increasing concentration of compounds. Media containing fresh compound was replenished every 3-4 days. At end of the incubation period, cells were stained with 0.1% crystal violent in 10% methanol for 10 minutes at room temperature. Stained plates were scanned and quantified using the Li-Cor Odyssey imaging system.

### Other Embodiments

In the claims articles such as “a.” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment disclosed herein that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope disclosed herein, as defined in the following claims.

What is claimed is:

1. A compound of Formula (I) or a pharmaceutically acceptable salt, hydrate, solvate, prodrug, stereoisomer, or tautomer thereof;
wherein: 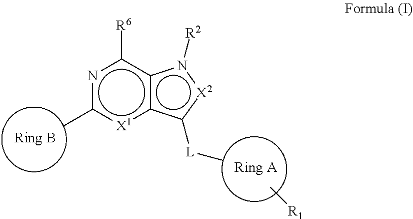
X¹ is selected from CH and N;
X² is selected from CR^{Xc2} and NR^{Xn2};
Ring B is a 5-6 member optionally substituted monocyclic aryl or heteroaryl;
L is selected from —O—, —NRⁿ—, —S—, —S(═O)—, —S(═O)₂— and —CR^{c}R^{c′};
Ring A is selected from C₆-C₁₀ aryl, 5-10 membered heteroaryl and 3-10 membered heterocyclyl, substituted with 0, 1 or 2 instances of halo or -Me;
R¹ is an optionally substituted 5-10 membered heteroaryl;
R² is absent or is selected from H, —C₁-C₆ alkyl, —C₃-C₉ cycloalkyl, —C₁-C₆ haloalkyl, —C₁-C₆ heteroalkyl, —C₁-C₆ hydroxyalkyl, arylalkyl, —S(═O)₂C₁-C₆alkyl and —C(═O)C₁-C₆alkyl, wherein each hydrogen of the alkyl, haloalkyl, heteroalkyl, hydroxylalkyl and arylalkyl can be independently replaced with a deuterium atom;
R⁶ is selected from H, -D, —CN, halo, —C₁-C₆ alkyl, —C₁-C₆ heteroalkyl, —C₁-C₆ haloalkyl, —C₃-C₁₀ cycloalkyl, —OH, and —O(C₁-C₆ alkyl);
each R^{Xc2} is independently selected from H,-D, halo, —C₁-C₆ alkyl, —C₁-C₆ heteroalkyl, —NH₂, —NH(C₁-C₆ alkyl), —O(C₁-C₆ alkyl) and —N(C₁-C₆ alkyl)₂;
each R^{Xn2} is absent or independently selected from H, —C₁-C₆ alkyl —C₁-C₆ haloalkyl, —S(═O)₂C₁-C₆alkyl and —C(═O)C₁-C₆alkyl;
each Rⁿ is independently selected from H and —C₁-C₆ alkyl; and
each R^{c} and R^{c′} is independently selected from H, —C₁-C₆ alkyl, —C₁-C₆ heteroalkyl, —C₁-C₆ haloalkyl, —OH, and —O(C₁-C₆ alkyl), or R^{c} and R^{c′} can be taken together with the atom to which they are attached to form a —C₃-C₄ cycloalkyl or a carbonyl.

2. The compound of claim 1 or a pharmaceutically acceptable salt, hydrate, solvate, prodrug, stereoisomer, or tautomer thereof, wherein Ring B is substituted with 0, 1, 2 or 3 instances of R^{b}, wherein
each R^{b} is independently selected from D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{b1}, —N(R^{b1})₂, —C(═O)R^{b1}, —C(═O)OR^{b1}, —NR^{b1}C(═O)R^{b1}, —NR^{b1}C(═O)OR^{b1}, —C(═O)N(R^{b1})₂, —OC(═O)N(R^{b1})₂, —S(═O)R^{b1}, —S(═O)₂R^{b1}, —SR^{b1}, —S(═O)(═NR^{b1})R^{b1}, —NR^{b1}S(═O)₂R^{b1} and —S(═O)₂N(R^{b1})₂ wherein each alkyl, cycloalkyl, heterocyclyl, aryl, heteroaryl, arylalkyl and heteroarylalkyl is optionally substituted at any available position;
each R^{b1} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl, wherein each hydrogen of the —C₁-C₆ alkyl of R^{b1} can be independently replaced with a deuterium atom.

3. The compound of claim 1 or 2, or a pharmaceutically acceptable salt, hydrate, solvate, prodrug, stereoisomer, or tautomer thereof, wherein each R^{b} is independently selected from —Cl, -ⁱPr, —CH₂N(CH₃)CH₂CH₃, —CH₂N(CH₃)₂, —CF₃, —CH₂OH, —CH(OH)(CH₃)₂, cyclopropyl (substituted with 0, 1 or 2 instances of —F), azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OCH₂F, —OⁱPr, —OPr, —OMe, —OCD₃, —OEt, —OH, —Ocyclopropyl, —N(Me)₂, —NHMe and —NHⁱPr.

4. The compound of any one of claims 1 to 3 or a pharmaceutically acceptable salt, hydrate, solvate, prodrug, stereoisomer, or tautomer thereof, wherein Ring B is selected from phenyl, pyrazolyl, pyridinyl and pyrimidinyl.

5. A compound of Formula (II) or a pharmaceutically acceptable salt, hydrate, solvate, prodrug, stereoisomer, or tautomer thereof;
wherein: 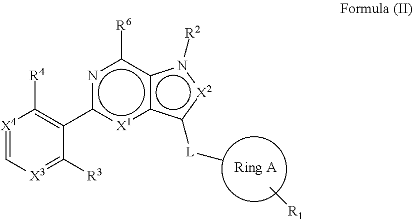
X¹ is selected from CH and N;
X² is selected from CR^{Xc2} and NR^{Xn2};
X³ is selected from CH and N;
X⁴ is selected from CH and N;
L is selected from —O—, —NRⁿ—, —S—, —S(═O)—, —S(═O)₂— and —CR^{c}R^{c′};
Ring A is selected from C₆-C₁₀ aryl, 5-10 membered heteroaryl and 3-10 membered heterocyclyl, each substituted with 0, 1 or 2 instances of halo or -Me;
R¹ is an optionally substituted 5-10 membered heteroaryl;
R² is absent or is selected from H, —C₁-C₆ alkyl, —C₃-C₉ cycloalkyl, —C₁-C₆ heteroalkyl, —C₁-C₆ haloalkyl, —C₁-C₆ hydroxyalkyl, arylalkyl, —S(═O)₂C₁-C₆alkyl and —C(═O)C₁-C₆alkyl, wherein each hydrogen of the alkyl, haloalkyl, heteroalkyl, hydroxylalkyl and arylalkyl can be independently replaced with a deuterium atom;
R³ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁ cycloalkyl, 3-10 membered heterocyclyl, heterocyclylalkyl, heteroarylalkyl, arylalkyl, cycloalkylalkyl, —OR, —N(R^{a3})₂, —C(═O)R^{a3}, —C(═O)OR^{a3}, —NR^{a3}C(═O)R^{a3}, —NRC(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position;
R⁴ is selected from H, D, halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position;
each R^{Xc2} is independently selected from H, —C₁-C₆ alkyl, —C₁-C₆ heteroalkyl, —NH₂, —NH(C₁-C₆ alkyl) and —N(C₁-C₆ alkyl)₂;
each R^{Xn2} is absent or independently selected from H, —C₁-C₆ alkyl, —C₁-C₆ haloalkyl, —S(═O)₂C₁-C₆alkyl and —C(═O)C₁-C₆alkyl;
each Rⁿ is independently selected from H and —C₁-C₆ alkyl;
each R^{c} and R^{c′} is independently selected from H, —C₁-C₆ alkyl, —C₁-C₆ heteroalkyl, —C₁-C₆ haloalkyl, —OH, and —O(C₁-C₆ alkyl), or R^{c} and R^{c′} can be taken together with the atom to which they are attached to form a —C₃-C₉ cycloalkyl or a carbonyl; and
each R^{a3} and R^{a4} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each hydrogen of the —C₁-C₆ alkyl can be independently replaced with a deuterium atom.

6. The compound of any one of claims 1 to 5 or a pharmaceutically acceptable salt, hydrate, solvate, prodrug, stereoisomer, or tautomer thereof, wherein each R^{Xc2} is independently selected from H, —F, -Me, —CH₂NMe₂, —CH₂NHMe, —CH₂OMe, —CH₂CH₂OMe.

7. The compound of claim 6 or a pharmaceutically acceptable salt, hydrate, solvate, prodrug, stereoisomer, or tautomer thereof, wherein R^{Xn2} is absent or is selected from —H, -Me, —CH₂CF₃, S(═O)₂Me, —C(═O)Me.

8. The compound of any one of claims 5 to 7 or a pharmaceutically acceptable salt, hydrate, solvate, prodrug, stereoisomer, or tautomer thereof, wherein the moiety represented by 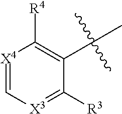
is selected from:

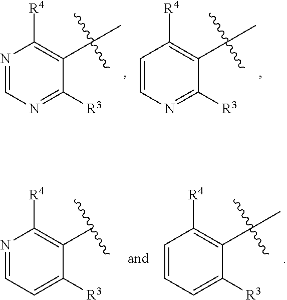

9. The compound of any one of claims 5 to 8, or a pharmaceutically acceptable salt, hydrate, solvate, prodrug, stereoisomer, or tautomer thereof, wherein each R³ is independently selected from H, D, halo, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{a3} and —N(R^{a3})₂, wherein each alkyl, cycloalkyl and heterocyclyl is substituted with 0, 1, 2 or 3 instances of halo, —OH, CN, -Me, -Et, —NH₂ or oxo and wherein each R is independently selected from H, —C₁-C₆ alkyl, —C₁-C₆ heteroalkyl, —C₁-C₆ haloalkyl and C₃-C₉ cycloalkyl, wherein each hydrogen atom of the C₁-C₆ alkyl of R^{a3} can be independently replaced by deuterium.

10. The compound of any one of claims 5 to 8, or a pharmaceutically acceptable salt, hydrate, solvate, prodrug, stereoisomer, or tautomer thereof, wherein each R³ is independently selected from H, -D, C₁, -ⁱPr, —CH₂N(CH₃)CH₂CH₃, —CH₂N(CH₃)₂, —CF₃, —CH₂OH, cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OCH₂F, —OⁱPr, —OPr, —OMe, —OCD₃, OEt, —OH, —Ocyclopropyl, —N(Me)₂, —NHMe and —NHⁱPr.

11. The compound of any one of claims 5 to 8, or a pharmaceutically acceptable salt, hydrate, solvate, prodrug, stereoisomer, or tautomer thereof, wherein R³ is —OMe.

12. The compound of any one of claims 5 to 11 or a pharmaceutically acceptable salt, hydrate, solvate, prodrug, stereoisomer, or tautomer thereof, wherein each R⁴ is independently selected from H, D, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —OR^{a4} and —N(R^{a4})₂, wherein each alkyl, cycloalkyl and heterocyclyl is substituted with 0, 1, 2 or 3 instances of halo, —OH, CN, -Me, -Et, —NH₂ or oxo and wherein each R^{a4} is independently selected from H, —C₁-C₆ alkyl, —C₁-C₆ heteroalkyl, —C₁-C₆ haloalkyl and C₃-C₉ cycloalkyl.

13. The compound of any one of claims 5 to 11, or a pharmaceutically acceptable salt, hydrate, solvate, prodrug, stereoisomer, or tautomer thereof, wherein each R⁴ is independently selected from H, -D, -ⁱPr, —CH₂N(CH₃)CH₂CH₃—, —CH₂N(CH₃)₂, —CF₃, —CH₂OH, cyclopropyl, azetidinyl (substituted with 0 or 1 instances of —F), 6-oxa-1-azaspiro[3.3]heptanyl, 6-oxa-1-azaspiro[3.4]octanyl), —OCH(CH₃)CF₃, —OCH₂CF₃, —OCHF₂, —OⁱPr, —OPr, —OMe, —OH, —Ocyclopropyl, —N(Me)₂, —NHMe and —NHⁱPr.

14. The compound of any one of claims 5 to 11, or a pharmaceutically acceptable salt, hydrate, solvate, prodrug, stereoisomer, or tautomer thereof, wherein each R⁴ is selected from H and cyclopropyl.

15. The compound of any one of claims 1 to 14 or a pharmaceutically acceptable salt, hydrate, solvate, prodrug, stereoisomer, or tautomer thereof, wherein L is —CR^{c}R^{c′}—.

16. The compound of claim 15 or a pharmaceutically acceptable salt, hydrate, solvate, prodrug, stereoisomer, or tautomer thereof, wherein R^{c} and R^{c′} are each independently selected from H, Me, —OH, —OMe or are taken together to form a carbonyl group or a cyclopropyl group.

17. The compound of any one of claims 1 to 16 or a pharmaceutically acceptable salt, hydrate, solvate, prodrug, stereoisomer, or tautomer thereof, wherein Ring A is a C₆-C₁₀ aryl or a 3-10 membered heterocyclyl containing 1 or 2 heteroatoms selected from N, O and S, each substituted with 0, 1 or 2 instances of halo or -Me.

18. The compound of any one of claims 1 to 17 or a pharmaceutically acceptable salt, hydrate, solvate, prodrug, stereoisomer, or tautomer thereof, wherein the moiety represented by 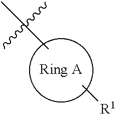
is selected from 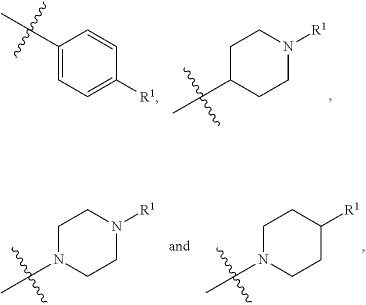
each independently substituted with 0, 1 or 2 instances of halo or -Me.

19. The compound of any one of claims 1 to 18 or a pharmaceutically acceptable salt, hydrate, solvate, prodrug, stereoisomer, or tautomer thereof, wherein the moiety represented by 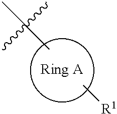
R¹ is 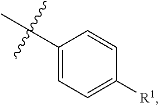
wherein the phenyl is further substituted with 0, 1 or 2 instances of halo or -Me.

20. The compound of any one of claims 1 to 19 or a pharmaceutically acceptable salt, hydrate, solvate, prodrug, stereoisomer, or tautomer thereof, wherein R¹ is a 5-10 membered heteroaryl substituted with 0, 1 or 2 instances of R⁵, wherein each R⁵ is independently selected from halo, —CN, —C₁-C₆ alkyl including deuterated versions thereof, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}S, —C(═O)N(R^{a5}a)₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂ wherein each alkyl, cycloalkyl, heterocyclyl, aryl, heteroaryl, arylalkyl and heteroarylalkyl is optionally substituted at any available position and wherein each R^{a5} is independently selected from H, —C₁-C₆ alkyl, —C₁-C₆ heteroalkyl, —C₁-C₆ haloalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl.

21. The compound of of claim 20 or a pharmaceutically acceptable salt, hydrate, solvate, prodrug, stereoisomer, or tautomer thereof, wherein R¹ is a 5 member monocyclic heteroaryl containing 1-3 heteroatoms selected from O, N and S.

22. The compound of of claim 21 or a pharmaceutically acceptable salt, hydrate, solvate, prodrug, stereoisomer, or tautomer thereof, wherein R¹ is selected from pyrazolyl and imidazolyl, each substituted with 0, 1 or 2 instances of R⁵.

23. The compound of any one of claims 20 to 22 or a pharmaceutically acceptable salt, hydrate, solvate, prodrug, stereoisomer, or tautomer thereof, wherein R⁵ is selected from CN, -Me, -CD₃, -Et, -ⁱPr, —CF₃, —OMe, —OEt, cyclopropyl, oxetanyl and azetidinyl.

24. The compound of any one of claims 1 to 23 or a pharmaceutically acceptable salt, hydrate, solvate, prodrug, stereoisomer, or tautomer thereof, wherein R¹ is selected from:

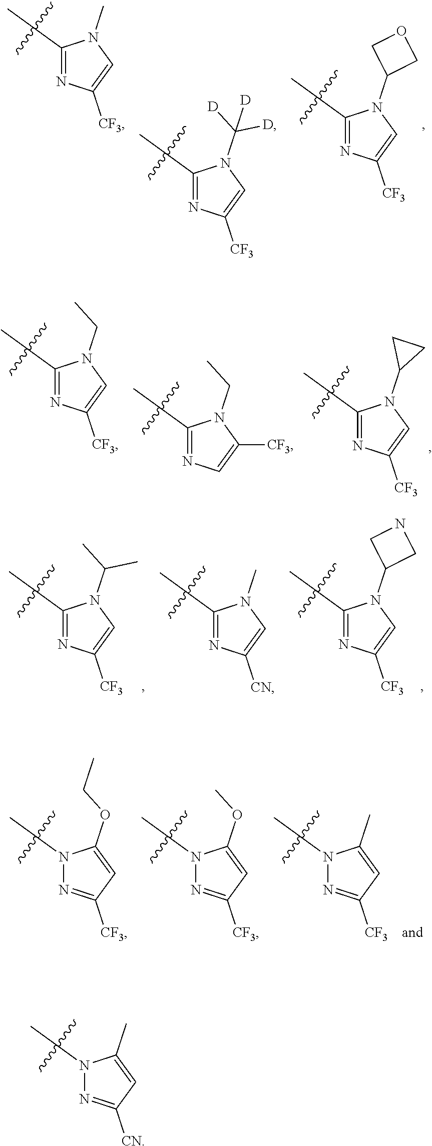

25. The compound of any one of claims 1 to 24 or a pharmaceutically acceptable salt, hydrate, solvate, prodrug, stereoisomer, or tautomer thereof, wherein R² is absent.

26. The compound of any one of claims 1 to 24 or a pharmaceutically acceptable salt, hydrate, solvate, prodrug, stereoisomer, or tautomer thereof, wherein R² is selected from H, -Me, -CD₃, -n-butyl, —CH₂CF₃, —S(═O)₂Me, —C(═O)Me, cyclopropyl, —CH₂CH₂OMe, —CH₂CH₂OH and benzyl.

27. The compound of any one of claims 1 to 24 or a pharmaceutically acceptable salt, hydrate, solvate, prodrug, stereoisomer, or tautomer thereof, wherein R² is H or -Me.

28. The compound of any one of claims 1 to 27 or a pharmaceutically acceptable salt, hydrate, solvate, prodrug, stereoisomer, or tautomer thereof, wherein R⁶ is selected from H, —F, Me and —OMe.

29. The compound of any one of claims 1 to 27 or a pharmaceutically acceptable salt, hydrate, solvate, prodrug, stereoisomer, or tautomer thereof, wherein R⁶ is H.

30. The compound of any one of claims 1 to 29 or a pharmaceutically acceptable salt, hydrate, solvate, prodrug, stereoisomer, or tautomer thereof, wherein the compound is selected from:

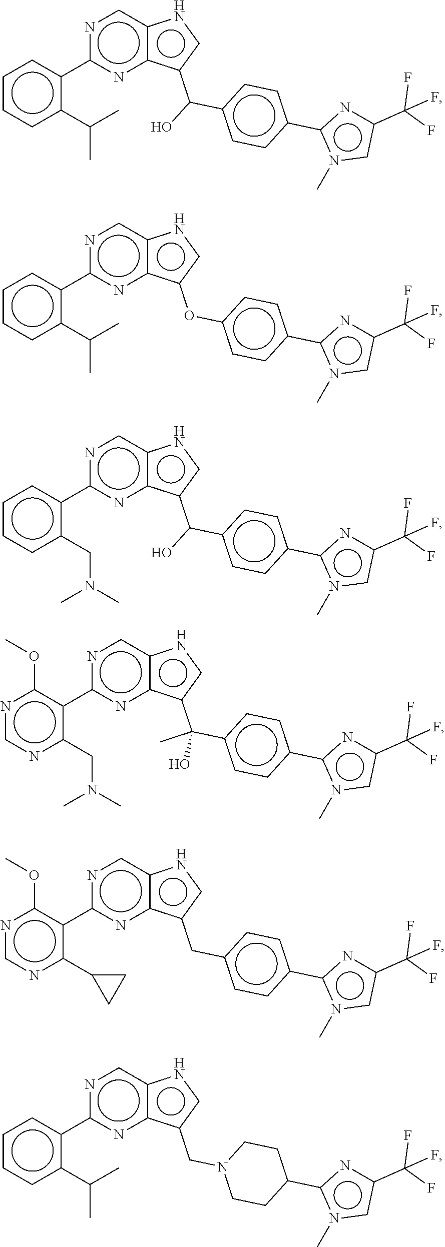

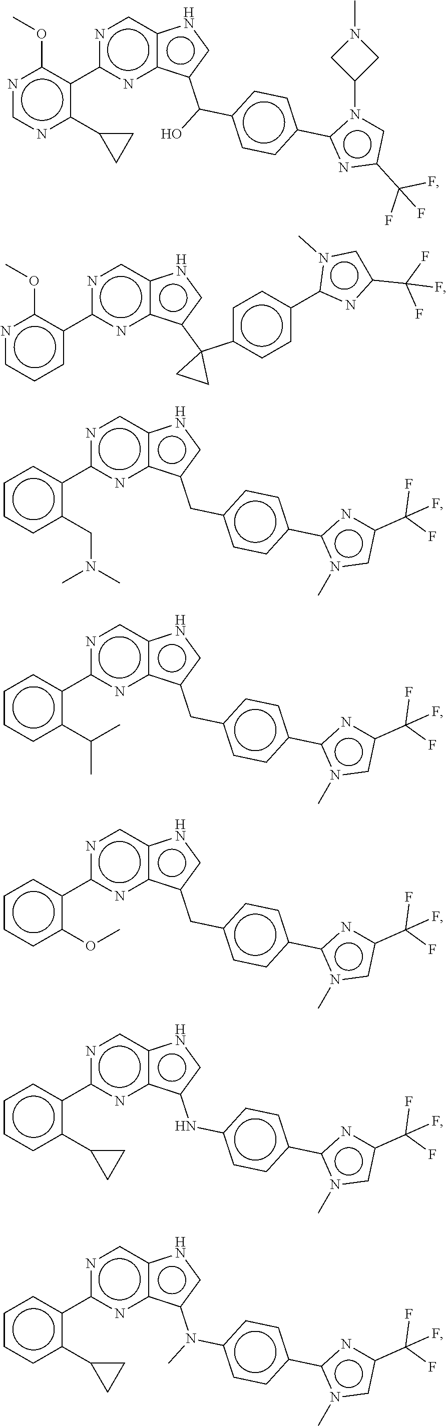

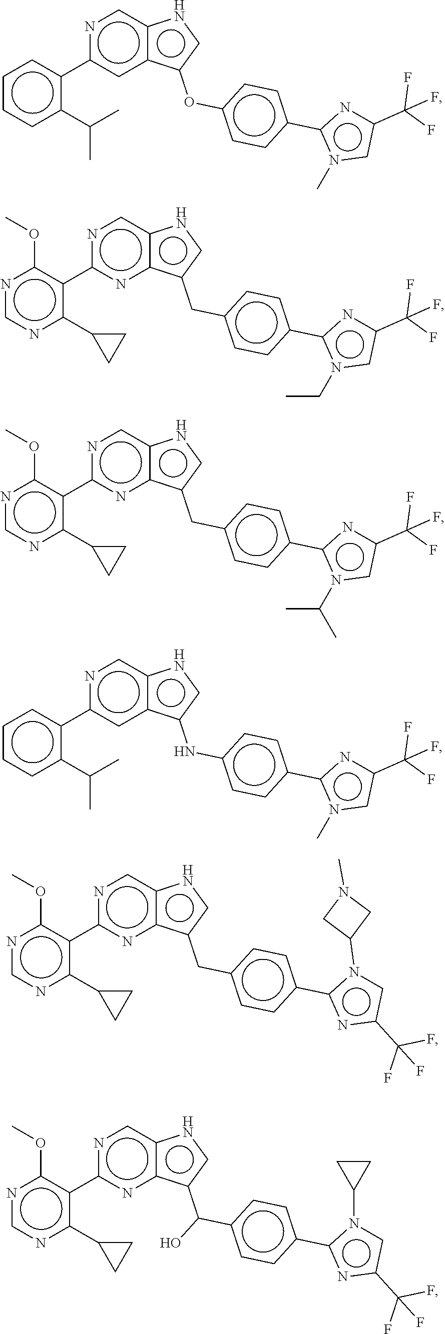

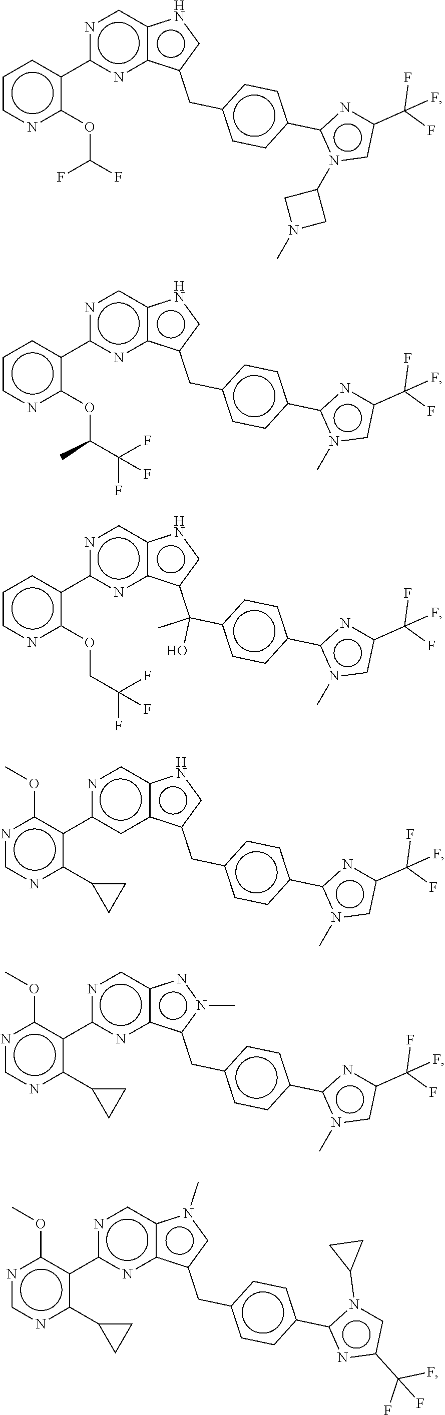

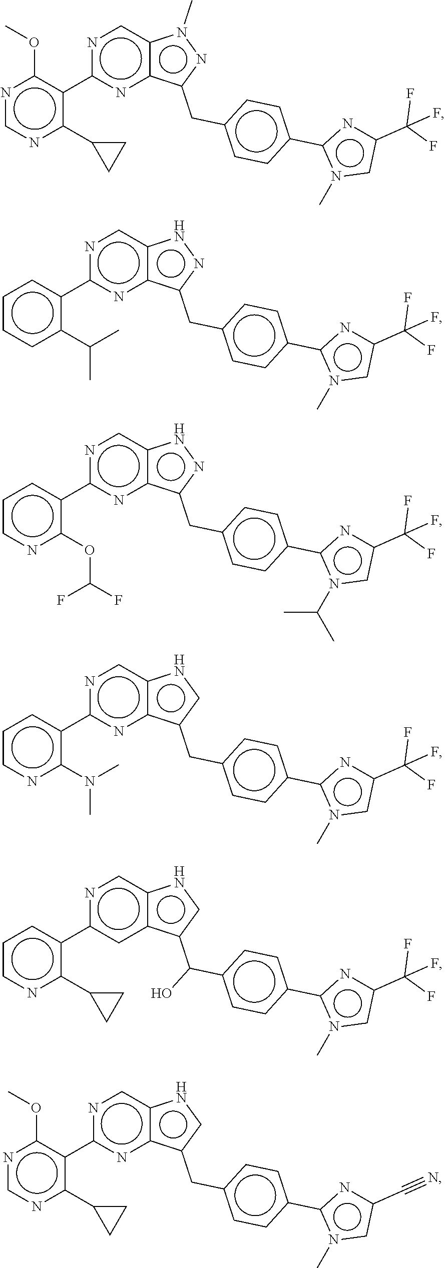

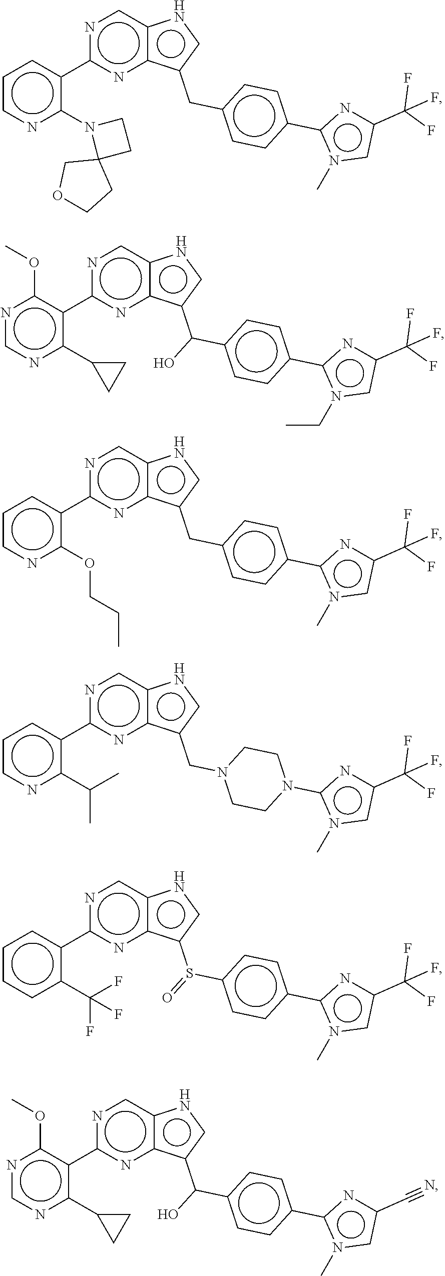

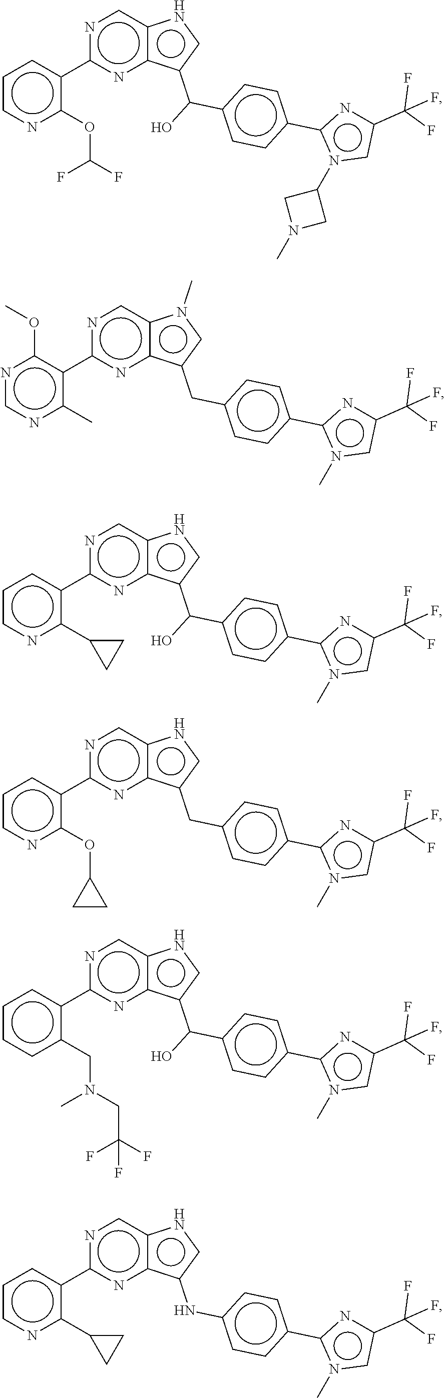

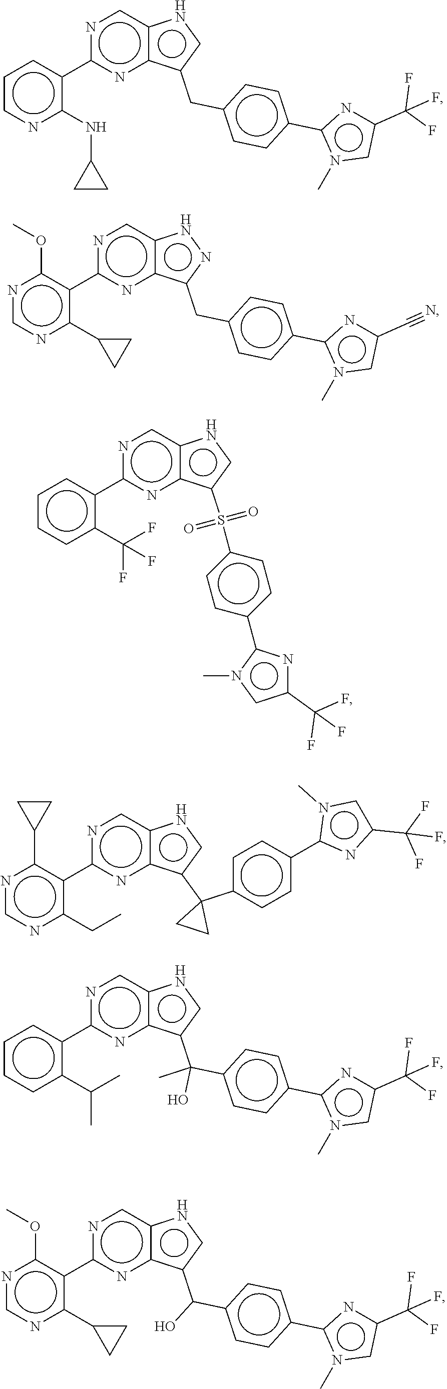

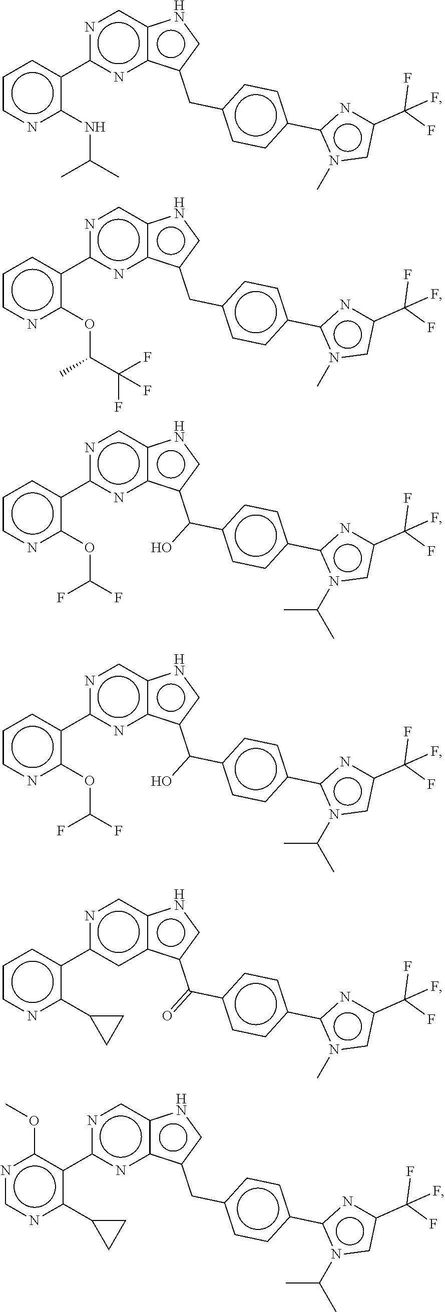

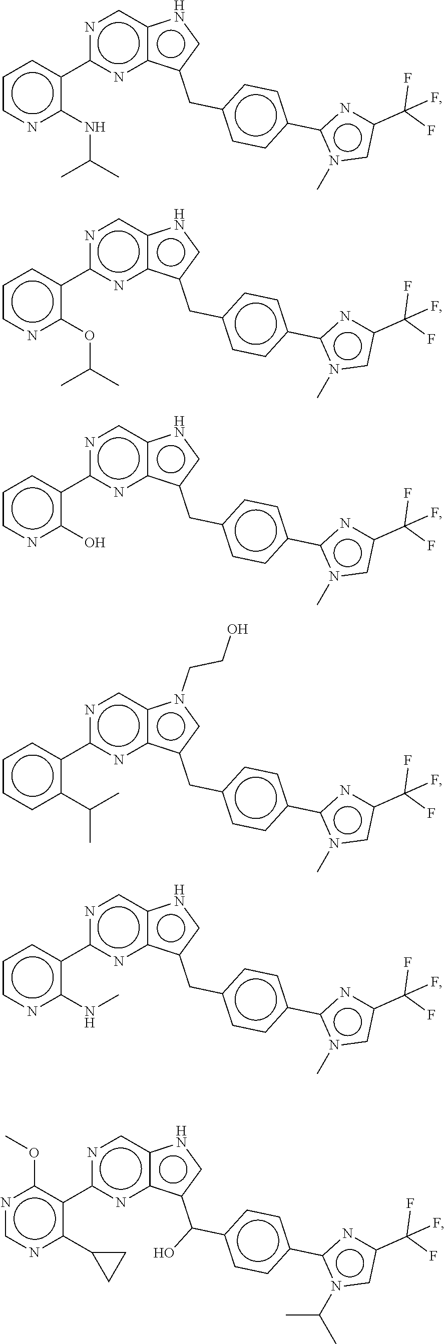

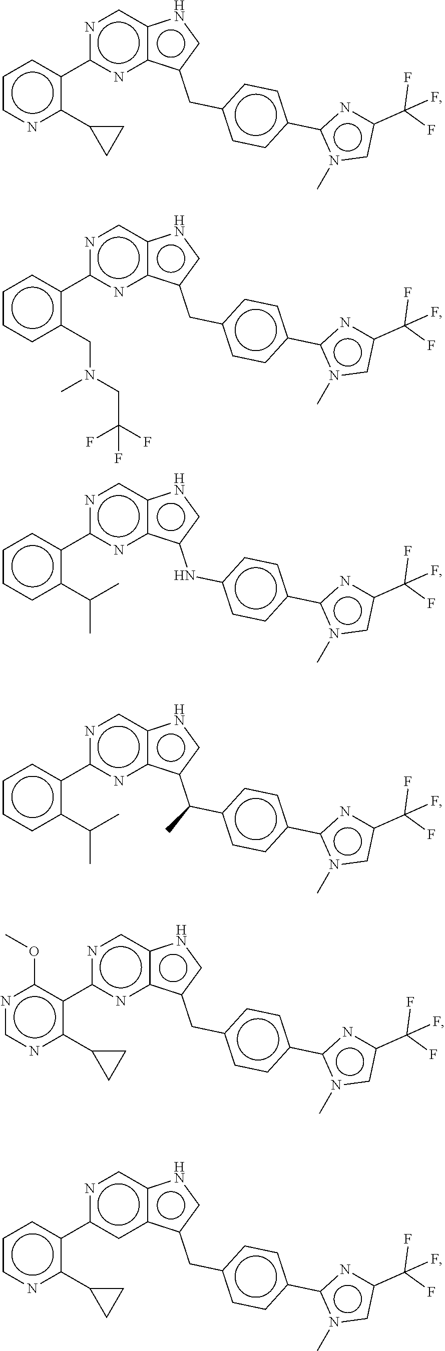

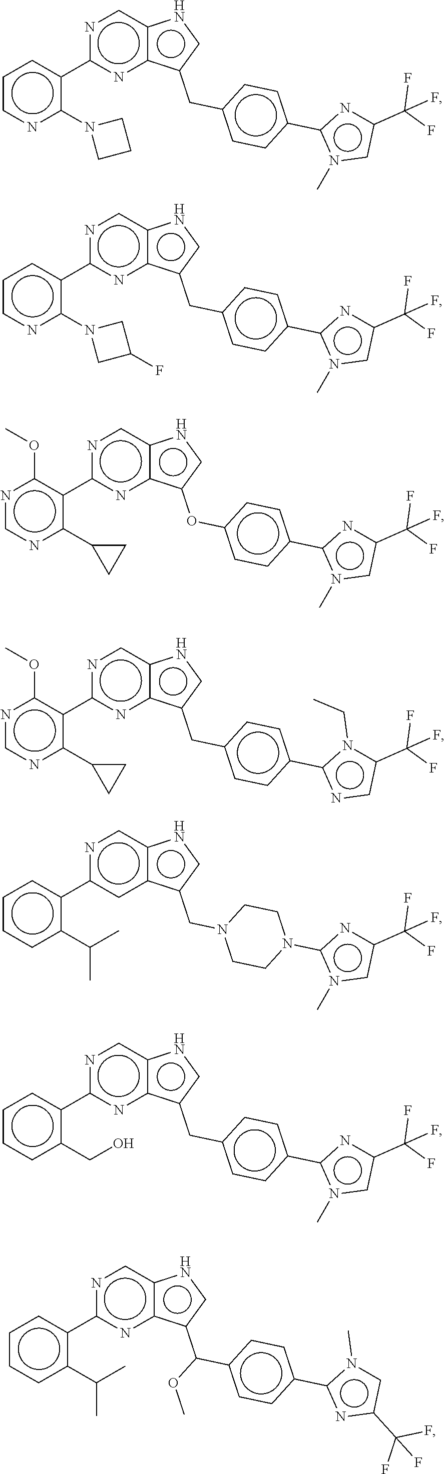

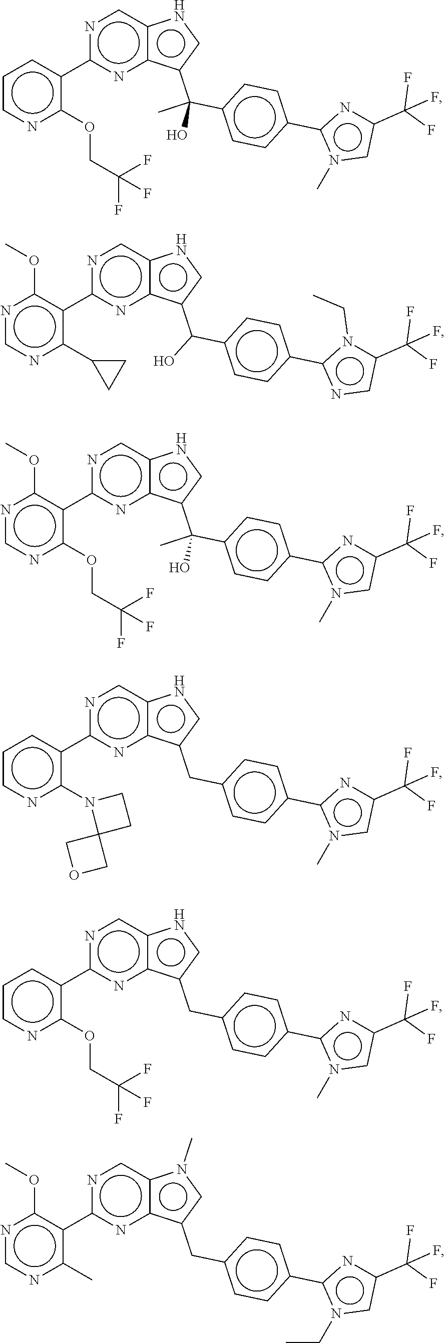

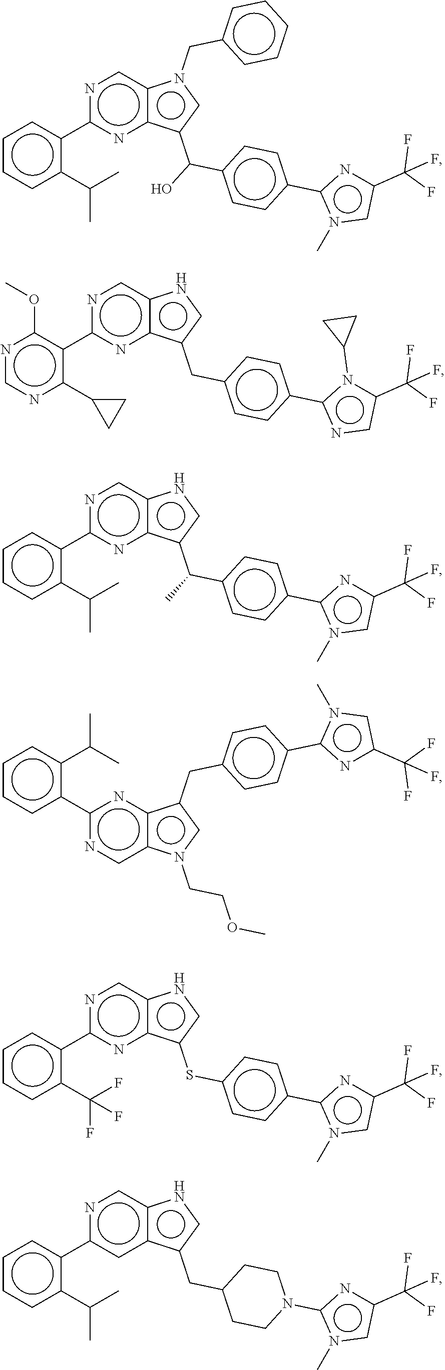

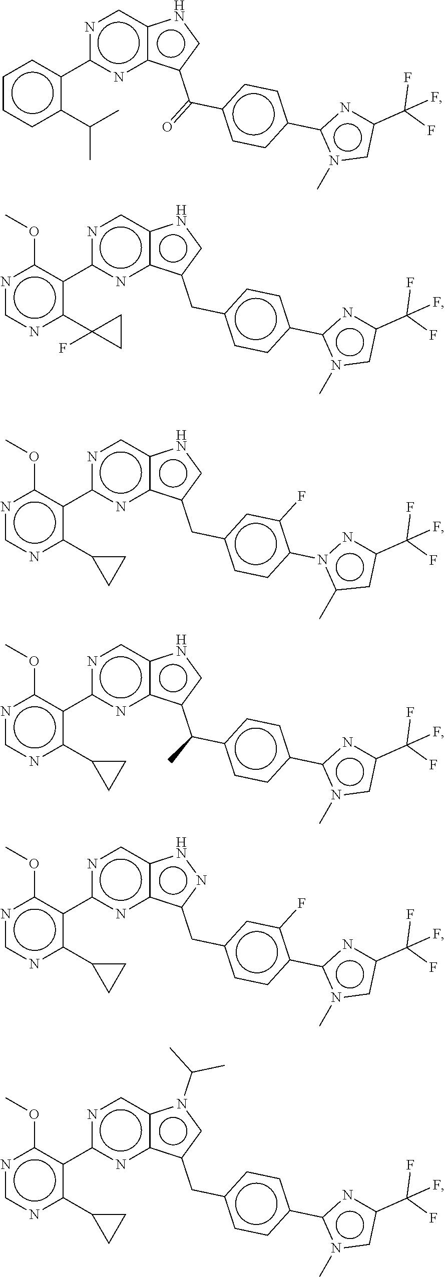

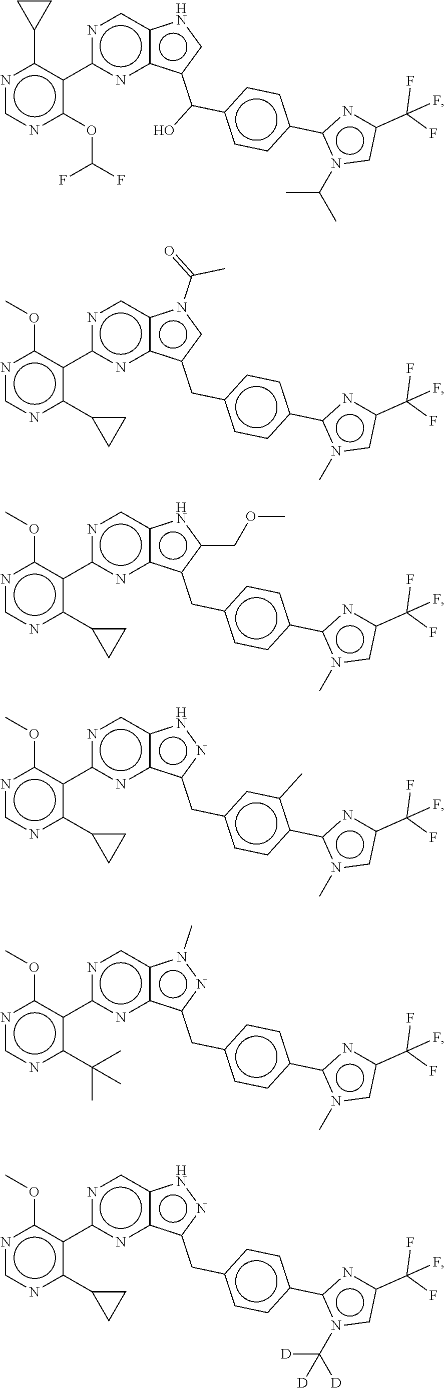

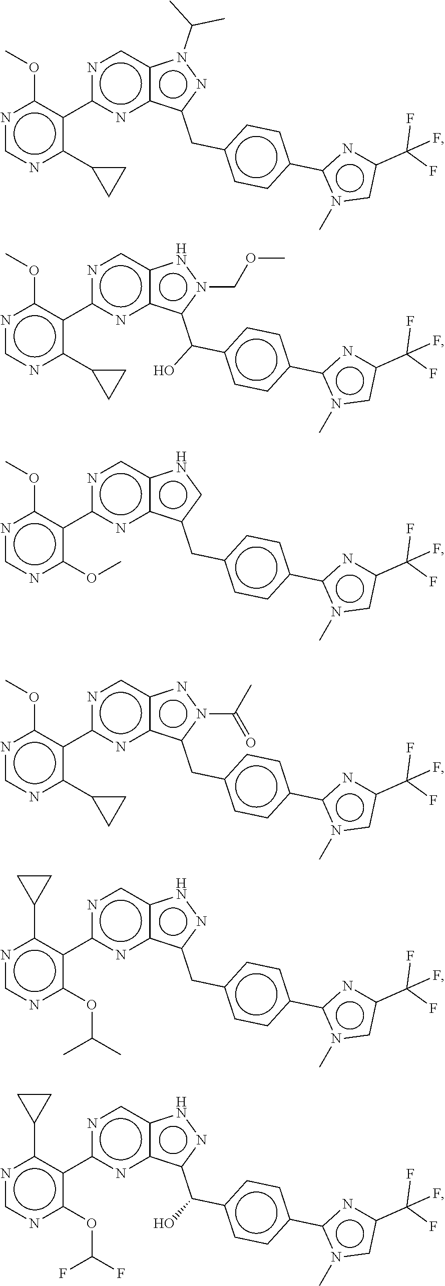

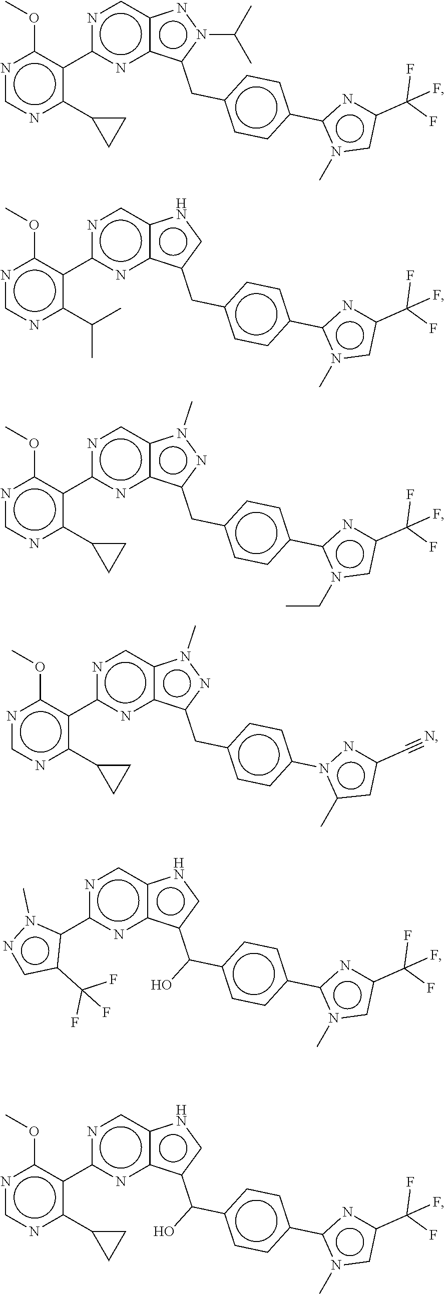

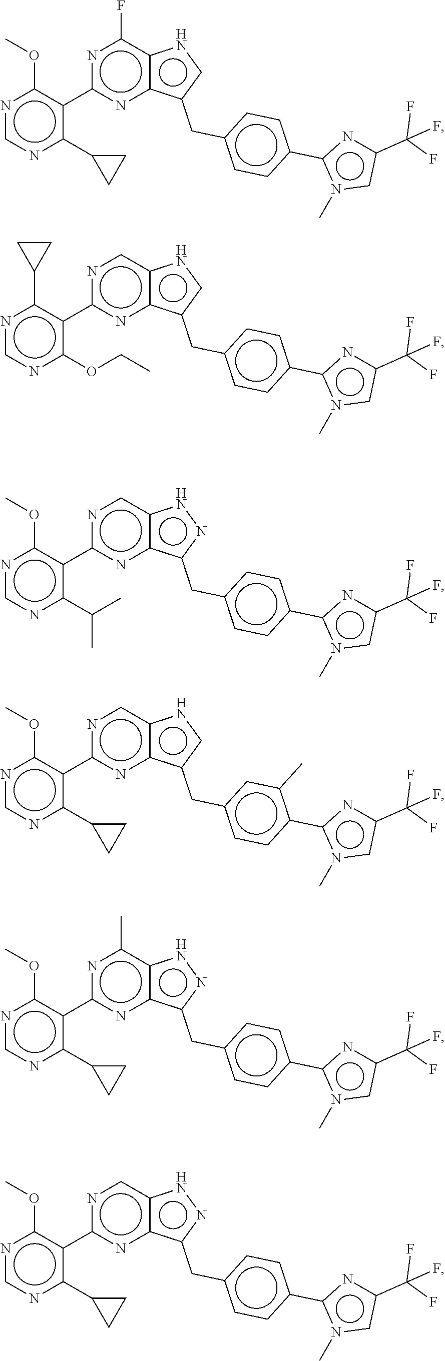

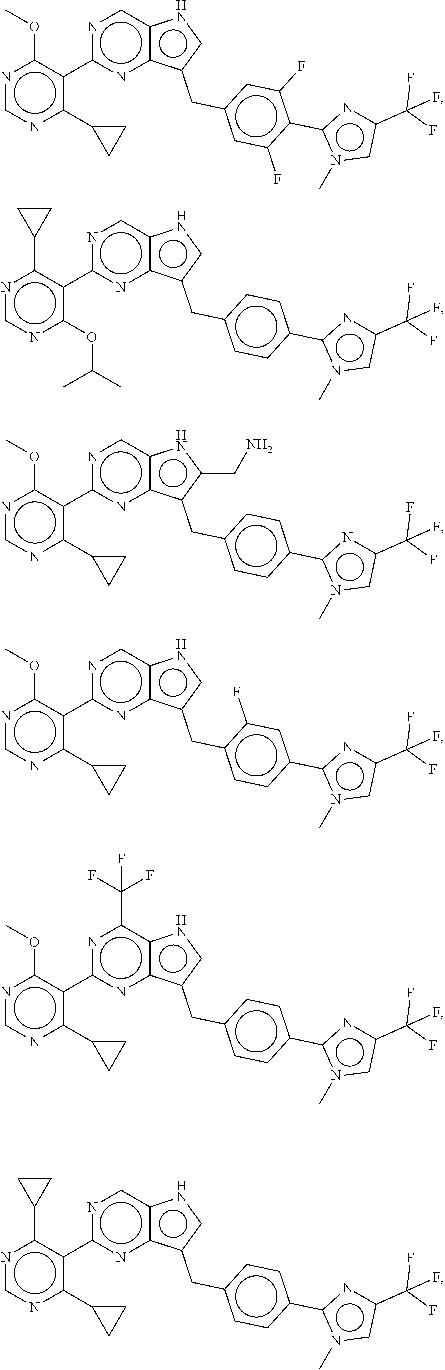

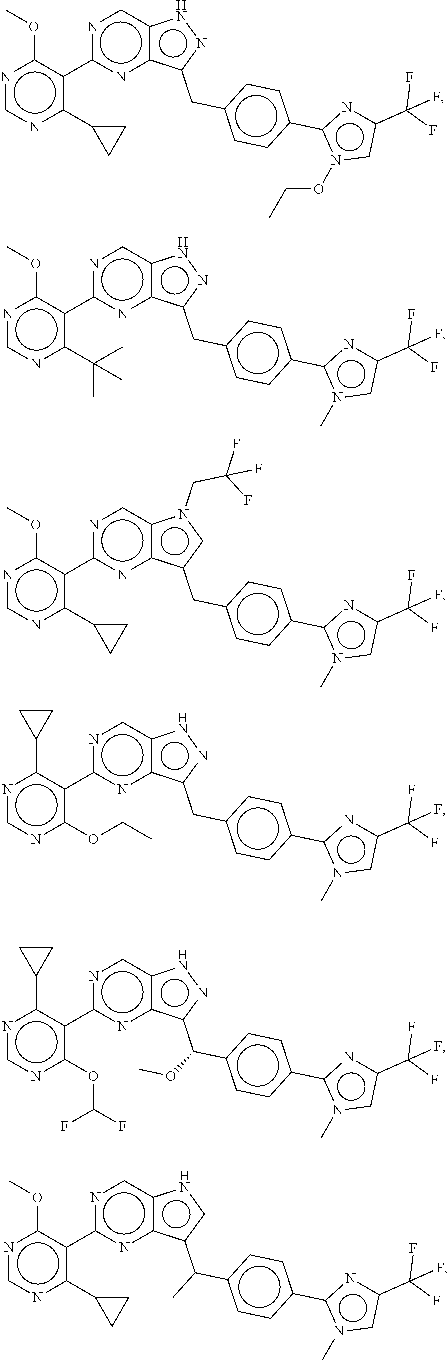

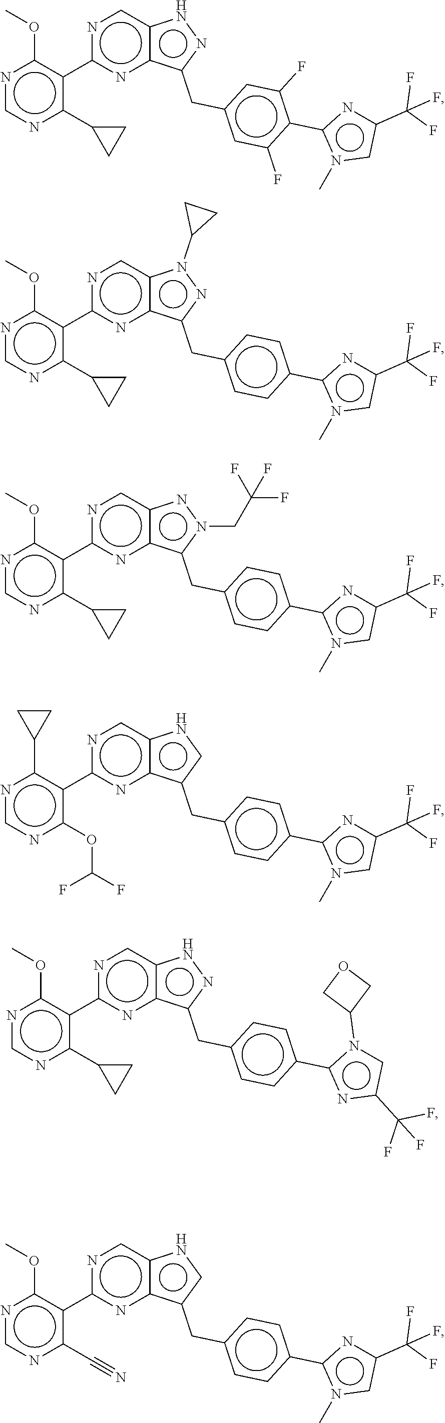

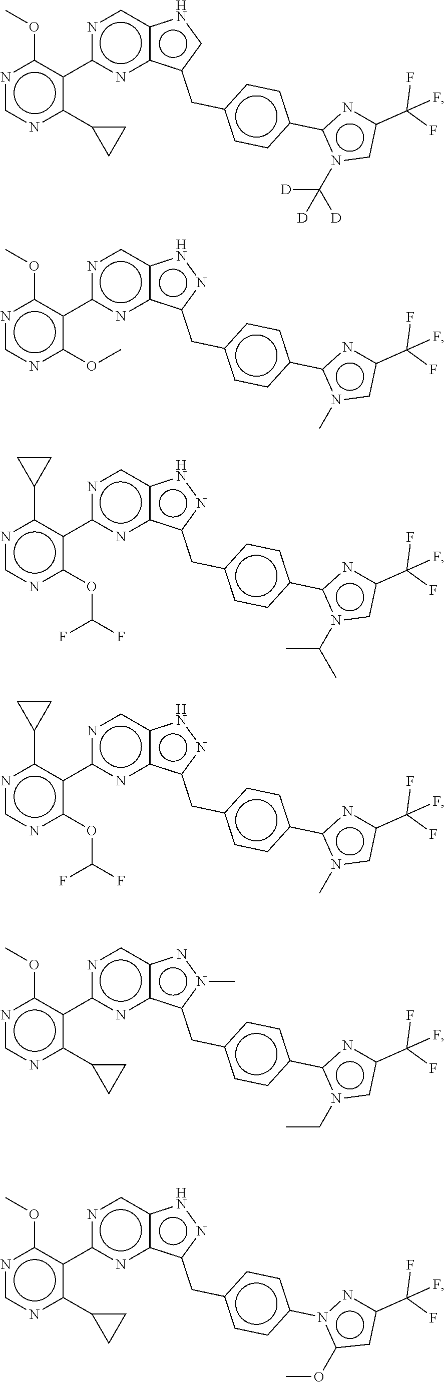

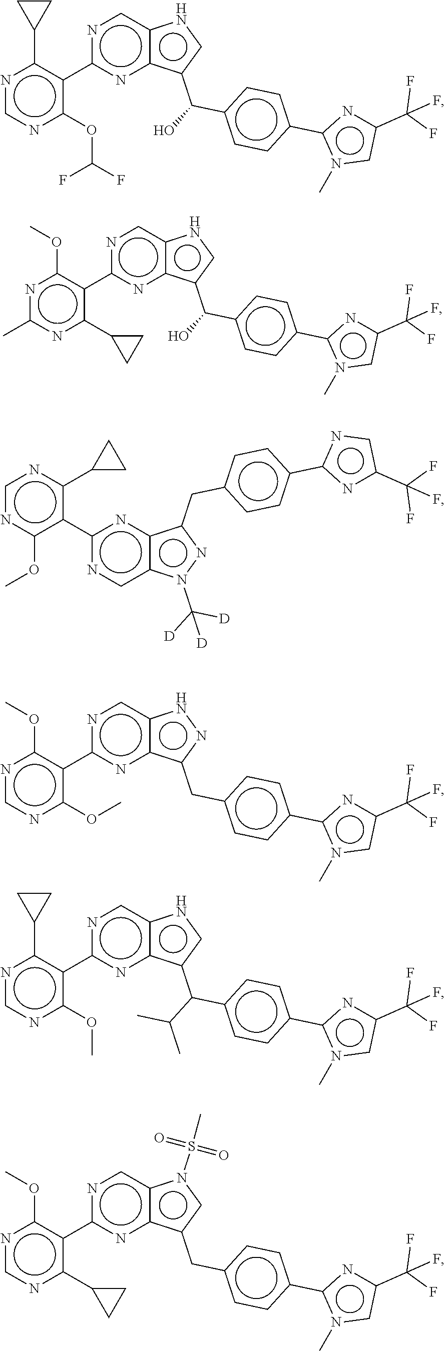

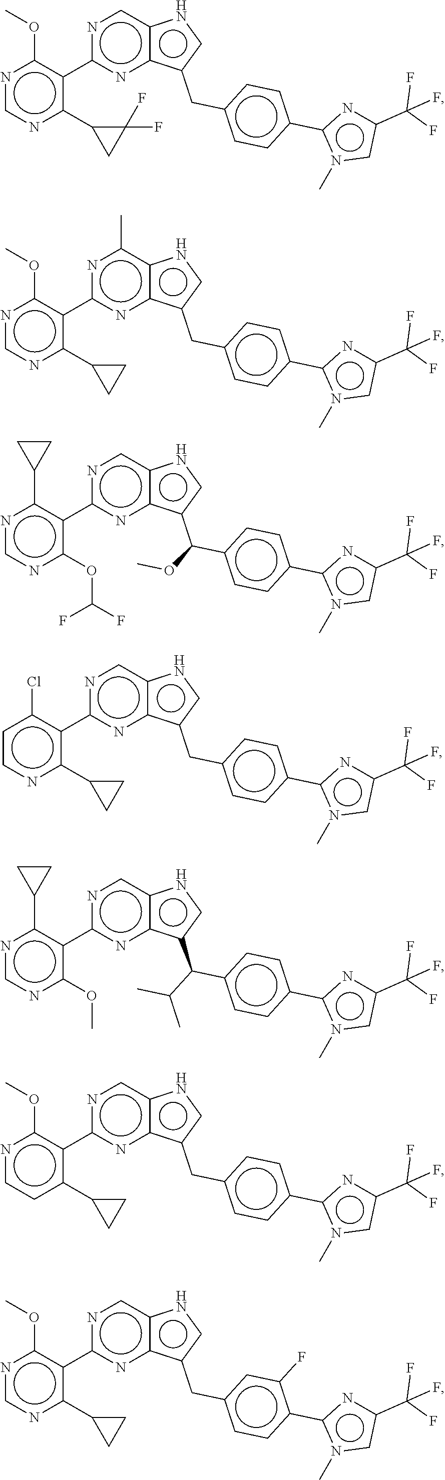

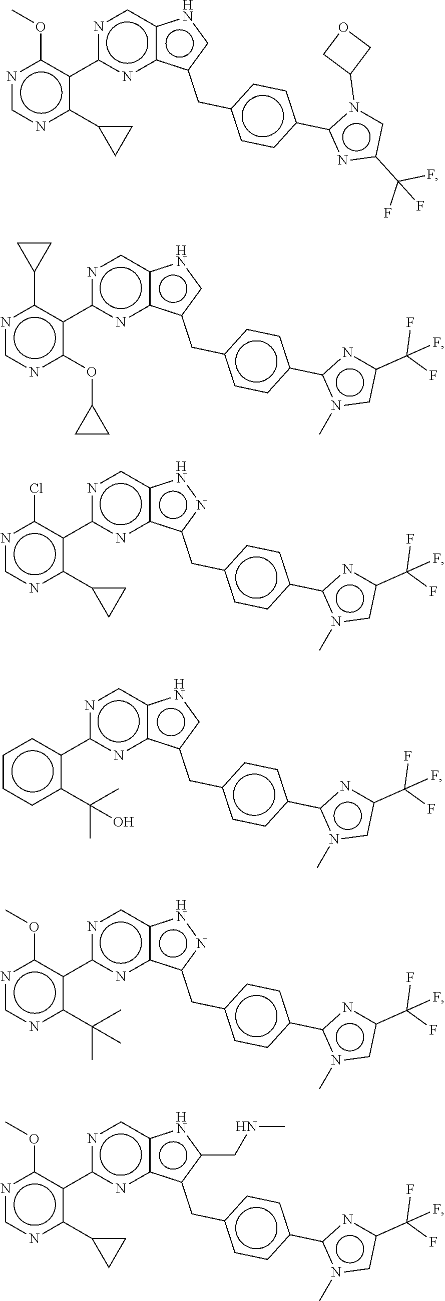

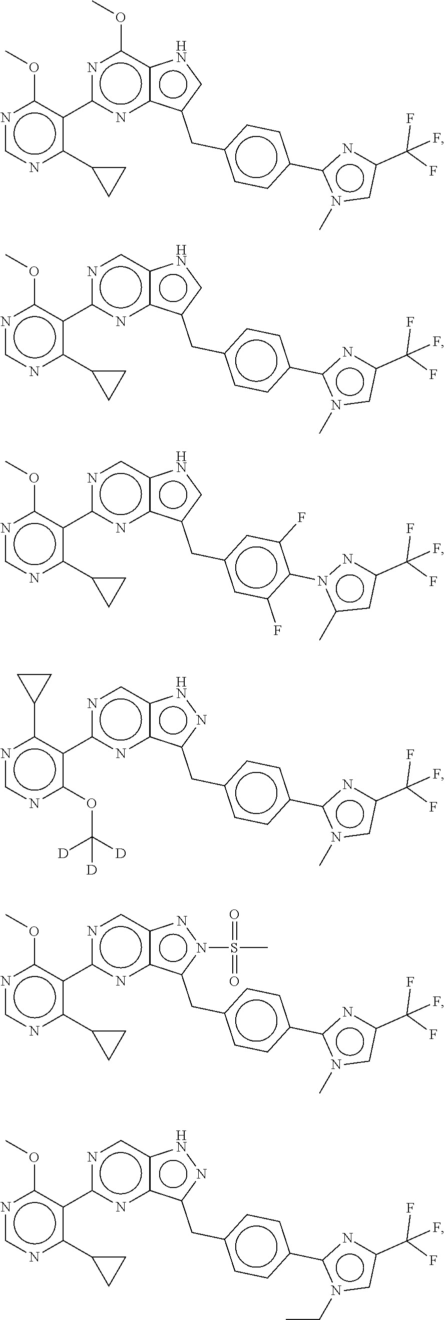

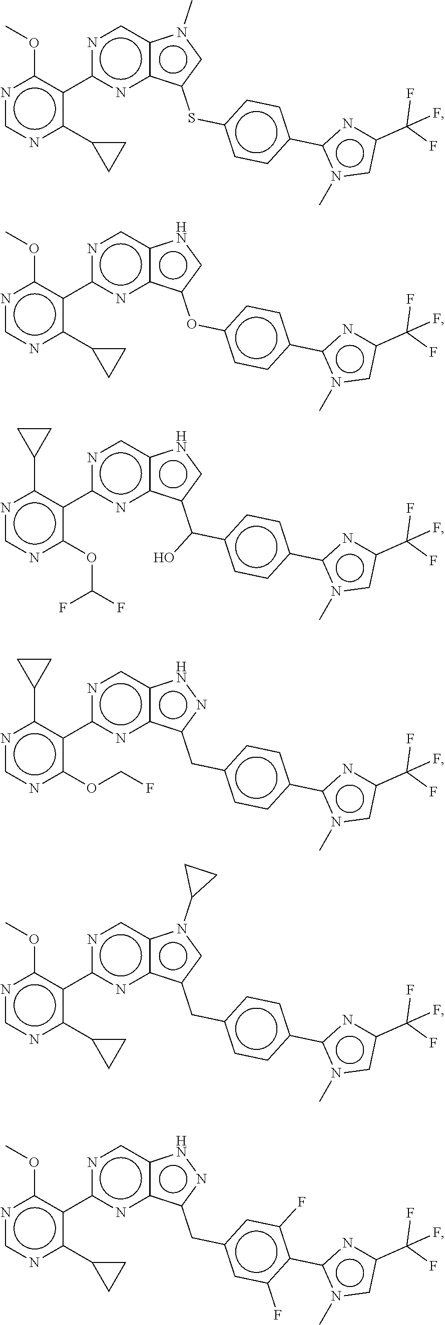

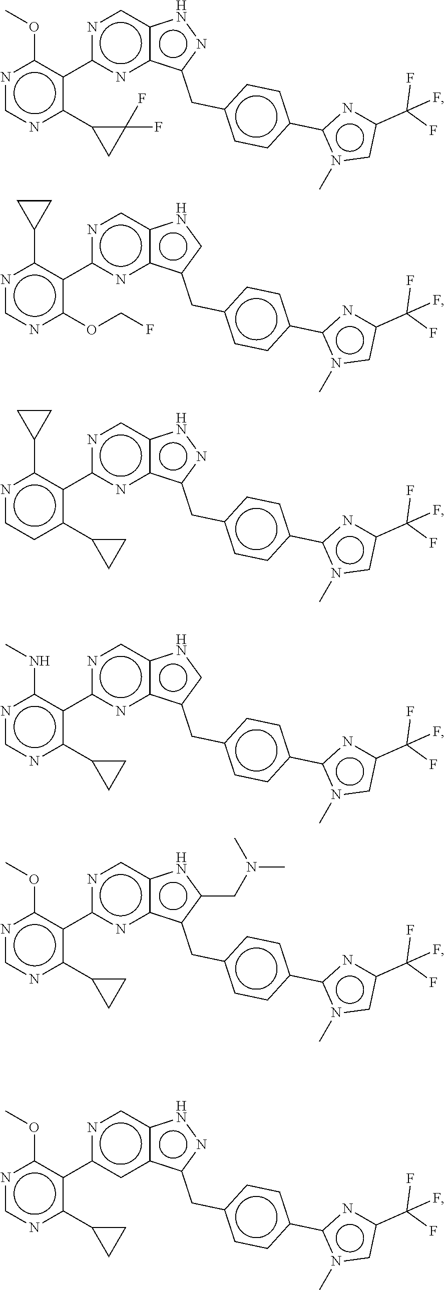

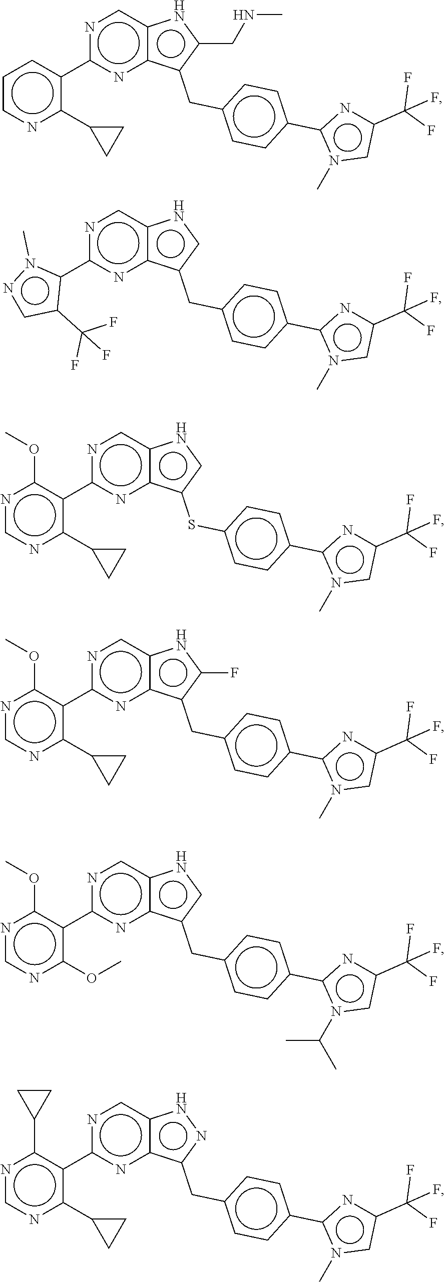

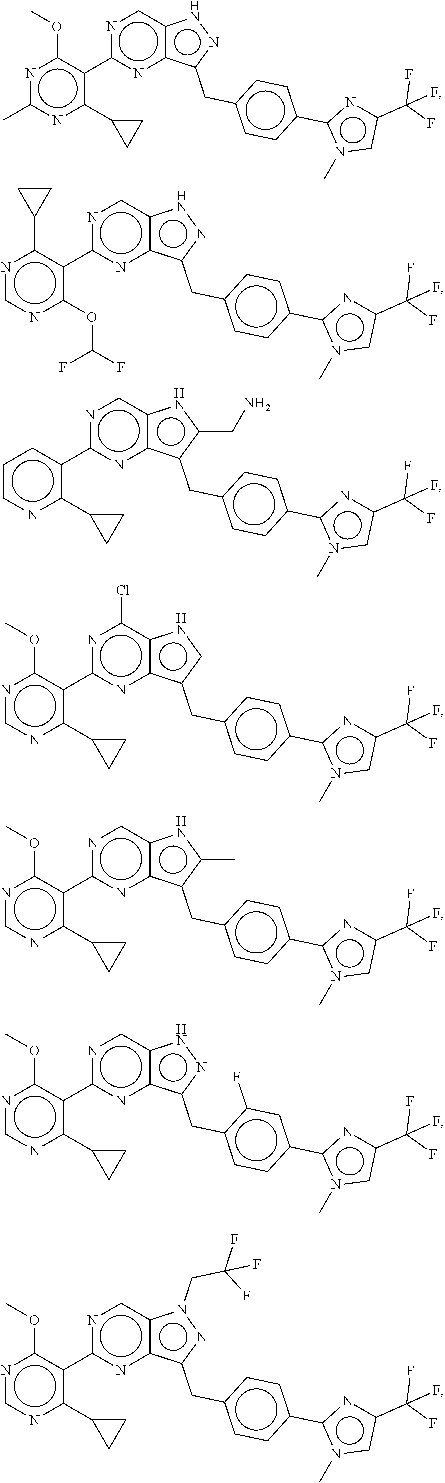

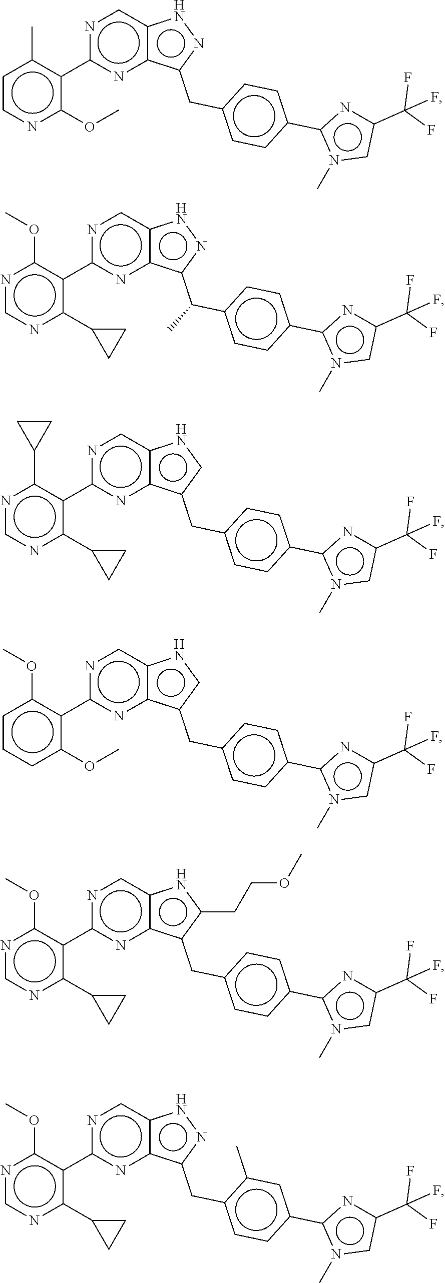

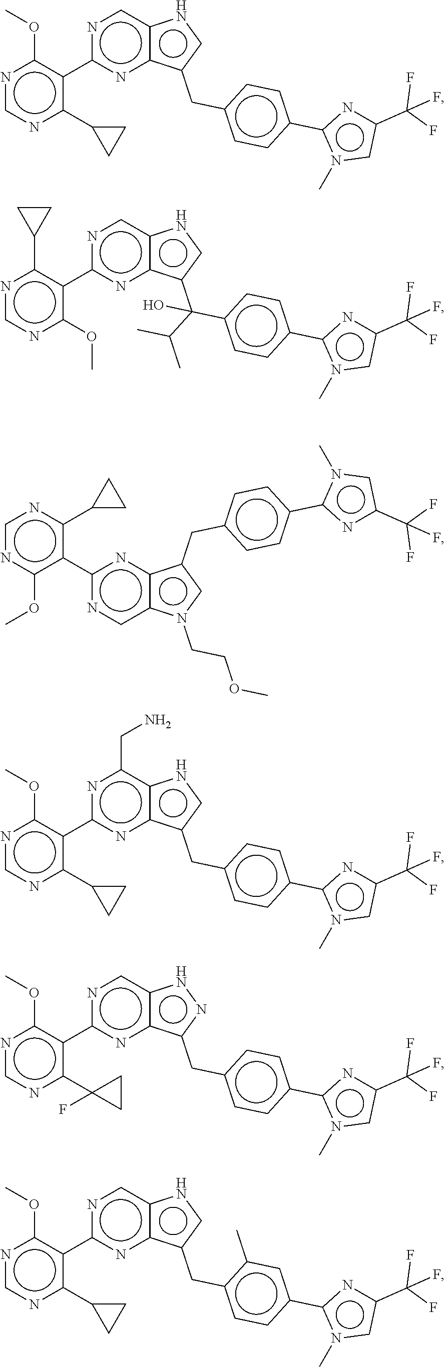

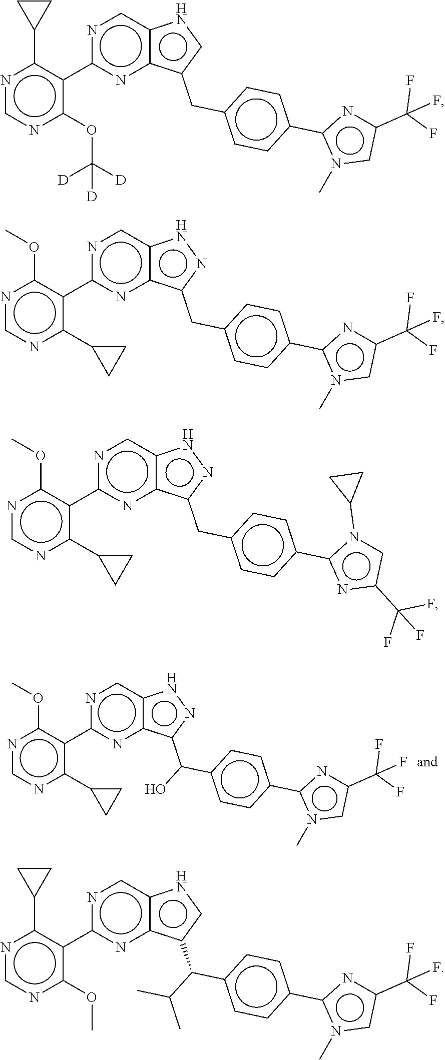

31. A pharmaceutical composition comprising a compound of any one of claims 1 to 30 or a pharmaceutically acceptable salt, hydrate, solvate, prodrug, stereoisomer, or tautomer thereof and a pharmaceutically acceptable carrier.

32. A compound of any one of claims 1-30 for use in a method for treating cancer in a patient in need thereof, wherein the method comprises administering to the patient an effective amount of the compound or a pharmaceutically acceptable salt, hydrate, solvate, prodrug, stereoisomer, or tautomer thereof.

33. A composition of claim 32 for use in a method for treating cancer in a patient in need thereof wherein the method comprises administering to the patient an effective amount of the composition.

34. The compound or composition for use of claim 32 or 33, wherein the cancer is a cancer that is sensitive to USP1 inhibition.

35. The compound or composition for use of claim 32 or 33 wherein the cancer is a BRCA1 and/or a BRCA2 mutant cancer.

36. The compound or composition for use of claim 32 or 33 wherein the cancer is a BRCA1 and/or a BRCA2 deficient cancer.

37. The compound or composition for use of claim 32 or 33 wherein the cancer is an ATM mutant cancer.

38. The compound or composition for use of claim 32 or 33 wherein the cancer is a PARP inhibitor resistant or refractory cancer.

39. The compound or composition for use of any one of claims 32 to 38, wherein the method comprises administering to the patient in need thereof an additional therapeutic agent.

40. The compound or composition for use of any one of claims 32 to 39 wherein the cancer is selected from adrenocortical carcinoma, AIDS-related lymphoma, AIDS-related malignancies, anal cancer, cerebellar astrocytoma, extrahepatic bile duct cancer, bladder cancer osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer, retinoblastoma, rhabdomyosarcoma, salivary gland cancer, malignant fibrous histiocytoma of bone, soft tissue sarcoma, Sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor.
